(19) United States
(12) Patent Application Publication
(10) Pub. No.: US 2011/0081315 A1
(43) Pub. Date: Apr. 7, 2011

(54) NOVEL MACROCYCLIC INHIBITORS OF HEPATITIS C VIRUS REPLICATION

(75) Inventors: Brad Buckman, Oakland, CA (US); John B. Nicholas, Redwood City, CA (US); Leonid Beigelman, San Mateo, CA (US); Vladimir Serebryany, Burlingame, CA (US); Antitsa Dimitrova Stoycheva, Half Moon Bay, CA (US); Timothy Thrailkill, San Francisco, CA (US); Scott Seiwert, Pacifica, CA (US)

(73) Assignee: INTERMUNE, INC., Brisbane, CA (US)

(21) Appl. No.: 12/890,475

(22) Filed: Sep. 24, 2010

Related U.S. Application Data
(60) Provisional application No. 61/246,465, filed on Sep. 28, 2009.
(60) Provisional application No. 61/324,251, filed on Apr. 14, 2010.
(60) Provisional application No. 61/345,737, filed on May 18, 2010.
(60) Provisional application No. 61/346,238, filed on May 19, 2010.

(51) Int. Cl. A61K 38/21; C07D 487/04; A61K 31/437; A61K 31/427; A61K 31/428; A61K 31/5377; A61K 31/445; A61K 31/506; A61K 31/4192; A61K 31/422; A61K 31/4725; A61K 31/522; A61K 31/498; C12N 9/99; C12N 5/02

(52) U.S. Cl. 424/85.5; 540/460; 514/303; 514/369; 514/367; 514/210.21; 514/233.2; 514/322; 514/256; 514/359; 514/374; 514/309; 514/263.2; 514/249; 435/184; 424/85.7; 435/375

(57) ABSTRACT

The embodiments provide compounds of the general Formulae I, Ia, II, III, IV, V, VI-1, VI-2, VII, VIII, IX, X, XI, and XII, as well as compositions, including pharmaceutical compositions, comprising a subject compound. The embodiments further provide treatment methods, including methods of treating a hepatitis C virus infection and methods of treating liver fibrosis, the methods generally involving administering to an individual in need thereof an effective amount of a subject compound or composition.

### RELATED APPLICATIONS

This application claims the benefit of U.S. Provisional Application Nos., 61/246,465, filed Sep. 28, 2009; 61/324,251, filed Apr. 14, 2010; 61/345,737, filed May 18, 2010; and 61/346,238, filed May 19, 2010; all of which are incorporated herein by reference in their entirety.

### BACKGROUND OF THE INVENTION

1. Field of the Invention

The present invention relates to compounds, processes for their synthesis, compositions and methods for the treatment of hepatitis C virus (HCV) infection.

2. Description of the Related Art

Hepatitis C virus (HCV) infection is the most common chronic blood borne infection in the United States. Although the numbers of new infections have declined, the burden of chronic infection is substantial, with Centers for Disease Control estimates of 3.9 million (1.8%) infected persons in the United States. Chronic liver disease is the tenth leading cause of death among adults in the United States, and accounts for approximately 25,000 deaths annually, or approximately 1% of all deaths. Studies indicate that 40% of chronic liver disease is HCV-related, resulting in an estimated 8,000-10,000 deaths each year. HCV-associated end-stage liver disease is the most frequent indication for liver transplantation among adults.

Antiviral therapy of chronic hepatitis C has evolved rapidly over the last decade, with significant improvements seen in the efficacy of treatment. Nevertheless, even with combination therapy using pegylated IFN-α plus ribavirin, 40% to 50% of patients fail therapy, i.e., are nonresponders (NR) or relapsers. These patients currently have no effective therapeutic alternative. In particular, patients who have advanced fibrosis or cirrhosis on liver biopsy are at significant risk of developing complications of advanced liver disease, including ascites, jaundice, variceal bleeding, encephalopathy, and progressive liver failure, as well as a markedly increased risk of hepatocellular carcinoma.

The high prevalence of chronic HCV infection has important public health implications for the future burden of chronic liver disease in the United States. Data derived from the National Health and Nutrition Examination Survey (NHANES III) indicate that a large increase in the rate of new HCV infections occurred from the late 1960s to the early 1980s, particularly among persons between 20 to 40 years of age. It is estimated that the number of persons with long-standing HCV infection of 20 years or longer could more than quadruple from 1990 to 2015, from 750,000 to over 3 million. The proportional increase in persons infected for 30 or 40 years would be even greater. Since the risk of HCV-related chronic liver disease is related to the duration of infection, with the risk of cirrhosis progressively increasing for persons infected for longer than 20 years, this will result in a substantial increase in cirrhosis-related morbidity and mortality among patients infected between the years of 1965-1985.

HCV is an enveloped positive strand RNA virus in the Flaviviridae family. The single strand HCV RNA genome is approximately 9500 nucleotides in length and has a single open reading frame (ORF) encoding a single large polyprotein of about 3000 amino acids. In infected cells, this polyprotein is cleaved at multiple sites by cellular and viral proteases to produce the structural and non-structural (NS) proteins of the virus. In the case of HCV, the generation of mature nonstructural proteins (NS2, NS3, NS4, NS4A, NS4B, NS5A, and NS5B) is effected by two viral proteases. The first viral protease cleaves at the NS2-NS3 junction of the polyprotein. The second viral protease is serine protease contained within the N-terminal region of NS3 (herein referred to as “NS3 protease”). NS3 protease mediates all of the subsequent cleavage events at sites downstream relative to the position of NS3 in the polyprotein (i.e., sites located between the C-terminus of NS3 and the C-terminus of the polyprotein). NS3 protease exhibits activity both in cis, at the NS3-NS4 cleavage site, and in trans, for the remaining NS4A-NS4B, NS4B-NS5A, and NS5A-NS5B sites. The NS4A protein is believed to serve multiple functions, acting as a cofactor for the NS3 protease and possibly assisting in the membrane localization of NS3 and other viral replicase components. Apparently, the formation of the complex between NS3 and NS4A is necessary for N53-mediated processing events and enhances proteolytic efficiency at all sites recognized by NS3. The NS3 protease also exhibits nucleoside triphosphatase and RNA helicase activities. NS5B is an RNA-dependent RNA polymerase involved in the replication of HCV RNA.

### SUMMARY OF THE INVENTION

The present embodiments provide compounds of the general Formula I or XII:

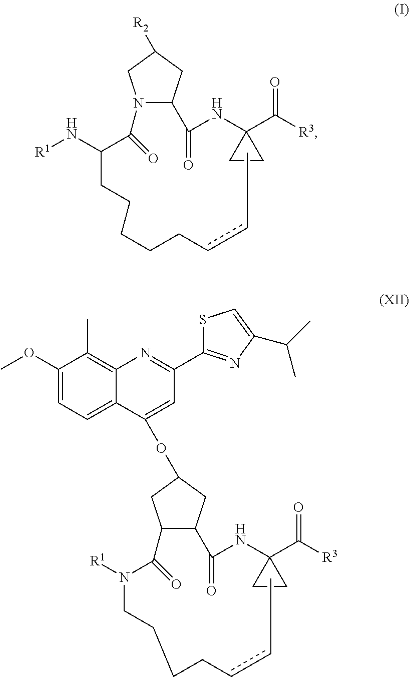

or a pharmaceutically acceptable salt or prodrug thereof wherein:

R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NCH(O)NR^{1a}R^{1b}, —C(O)OR^{1c}, and heteroaryl.

R^{1e} is selected from the group consisting of t-butyl, cycloalkyl, and heterocyclyl.

R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl.

R^{1c} and R^{1d} are each separately selected from the group consisting of —H, C₁₋₄ alkoxy, C₁₋₆ alkyl, C₃₋₇ cycloalkyl, aryl, arylalkyl and heteroaryl.

R² is selected from the group consisting of

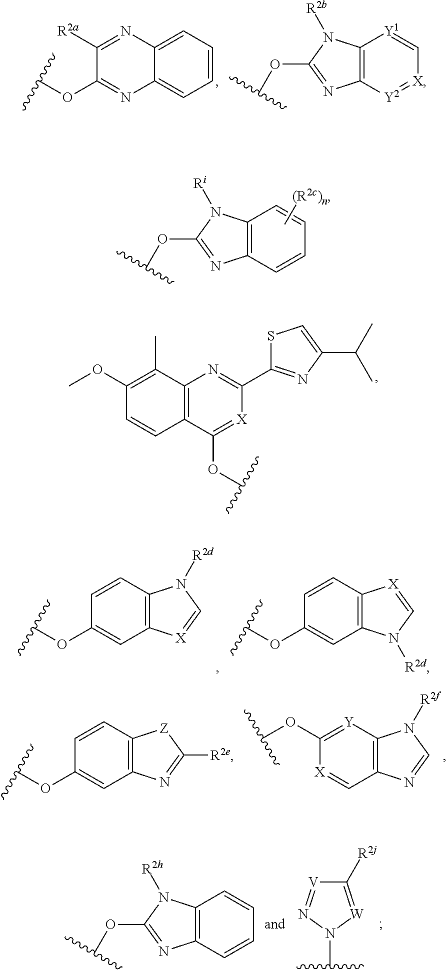

X, Y, Y¹, and Y² are each independently selected from —CH— or —N—, wherein X and Y are not both —CH—, and X, Y¹, and Y² are not all —CH—; Z is O (oxygen) or S (sulfur); V and W are each independently selected from —CR^{2k}— or —N—, wherein V and W are not both —CR^{2k}—; n is 1, 2 or 3; and R^{2j} and R^{2k} are each independently selected from the group consisting of H, halo, optionally substituted aryl, optionally substituted heteroaryl; or R^{2j} and R^{2k} together form an aryl ring optionally substituted by 1-3 R^{2g}.

R^{2a}, R^{2e} and R^{2g} are each independently selected from the group consisting of halo, —C(O)OR^{1c}, —C(O)NR′R″, —NR′R″, —NHC(O)NR′R″, —NHC(O)OR^{1c}, —NHS(O)₂R^{1c}, C₁₋₆ alkyl optionally substituted with up to 5 fluoro, C₂₋₆ alkenyl, C₃₋₇ cycloalkyl, optionally substituted C₁₋₆ alkoxy, optionally substituted aryl and optionally substituted heteroaryl.

Each R^{2c} is independently selected from the group consisting of halo, —C(O)OR^{1c}, —C(O)NR′R″, —NR′R″, —NHC(O)NR′R″, —NHC(O)OR^{1c}, —NHS(O)₂R^{1c}, C₁₋₆ alkyl, C₂₋₆ alkenyl, C₃₋₇ cycloalkyl, C₁₋₆ alkoxy, arylalkyl, polycyclic moiety, aryl, and heteroaryl, wherein said C₁₋₆ alkyl, C₂₋₆ alkenyl, C₃₋₇ cycloalkyl, C₁₋₆ alkoxy, arylalkyl, polycyclic moiety, aryl, and heteroaryl each optionally substituted with one or more R¹². Each R¹² is independently selected from the group consisting of C₁₋₆ alkyl, C₃₋₇ cycloalkyl, C₁₋₆ alkoxy, heteroaryl, arylalkyl, aryl, —F (fluoro), —Cl (Chloro), —CN, —CF₃, —OCF₃, —C(O)NR′R″ and —NR′R″, wherein said C₁₋₆ alkyl, C₃₋₇ cycloalkyl, C₁₋₆ alkoxy, heteroaryl, arylalkyl, cycloalkylalkyl, and aryl are each optionally substituted with one or more R^{12a}. Each R^{12a} is independently selected from the group consisting of —F, —Cl, —CF₃, —OCF₃, C₁₋₆ alkyl, C₁₋₆ alkoxy, C₃₋₇ cycloalkyl, and aryl.

Each NR′R″ is separately selected wherein R′ and R″ are each independently selected from the group consisting of —H (hydrogen), halo, —C(O)NR′R″, optionally substituted C₁₋₆ alkyl, optionally substituted C₂₋₆ alkenyl, optionally substituted C₁₋₆ alkoxy, optionally substituted aryl, optionally substituted arylalkyl and optionally substituted heteroaryl; or R′ and R″ are taken together with the nitrogen to which they are attached to form heterocyclyl.

R^{2b}, R^{2d}, and R^{2f} are each independently selected from the group consisting of C₁₋₆ alkyl optionally substituted with up to 5 fluoro, C₂₋₆ alkenyl, C₃₋₇ cycloalkyl, arylalkyl, optionally substituted aryl and optionally substituted heteroaryl; R^{2h} is selected from the group consisting of propyl, butyl and phenyl; Rⁱ is C₁₋₆ alkyl optionally substituted with up to 5 fluoro.

R³ is —OH, —NHS(O)₂R^{3a}, —NHS(O)₂OR^{3a} or —NHS(O)₂NR^{3b}R^{3c}; where R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro.

R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{3b} and R^{3c} are taken together with the nitrogen to which they are attached to form a three- to six-membered heterocyclic ring, bonded to the parent structure through a nitrogen, and the heterocylic ring is optionally substituted with one or more substituents each independently selected from the group consisting of halo, cyano, nitro, C₁₋₆ alkyl, C₁₋₆ alkoxy, and phenyl; each t is independently 0, 1 or 2; and each q is independently 0, 1 or 2.

Any bond represented by a dashed and solid line represents a bond selected from the group consisting of a single bond and a double bond.

Provided that if R² is

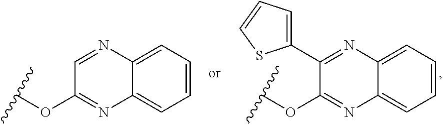

then R¹ is not phenyl.

Provided that if R² is

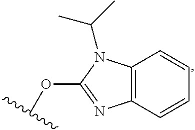

then R¹ is not —C(O)O-t-butyl, phenyl or phenyl substituted with one or more substituents selected from the group consisting of fluoro, chloro and —CF₃.

Provided that if R² is

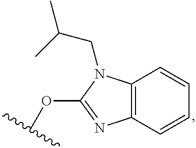

then R¹ is not —C(O)O-t-butyl or phenyl substituted with one or more substituents selected from the group consisting of fluoro and —CF₃.

Provided that if R² is

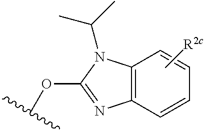

and R^{2c} is —F or methyl, then R¹ is not —C(O)O-t-butyl or phenyl.

Provided that if R² is

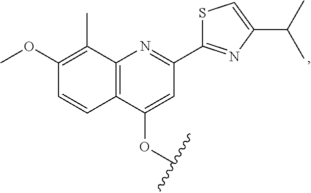

then R¹ is not —C(O)O-t-butyl, benzoxazyl, t-butylthiazyl, phenyl or phenyl substituted with one or more substituents selected from the group consisting of fluoro, chloro, methyl, —CF₃ and —OCF₃.

Some embodiments provide a compound having the structure of Formula IIa-1:

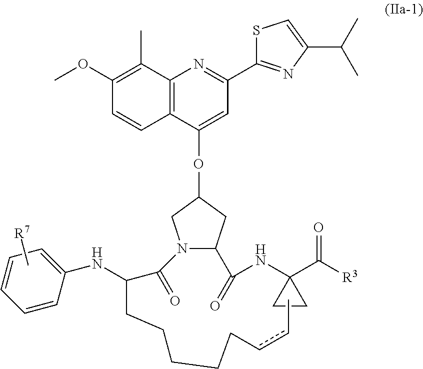

or a pharmaceutically acceptable salt or prodrug thereof wherein R³ is —OH, —NHS(O)₂R^{3a}, —NHS(O)₂OR^{3a} or —NHS(O)₂NR^{3b}R^{3c}; where R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro.

R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{3b} and R^{3c} are taken together with the nitrogen to which they are attached to form a three- to six-membered heterocyclic ring, bonded to the parent structure through a nitrogen, and the heterocylic ring is optionally substituted with one or more substituents each independently selected from the group consisting of halo, cyano, nitro, C₁₋₆ alkyl, C₁₋₆ alkoxy, and phenyl.

Each t is independently 0, 1 or 2; and each q is independently 0, 1 or 2.

R⁷ is selected from the group consisting of —NH₂, —NH₂.HCl, —COOH, —C(O)NR^{1a}R^{1b}, —NHC(O)NR^{1a}R^{1b} and heteroaryl containing 1-3 heteroatoms independently selected from N or O; R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl; R^{1c} and R^{1d} are each separately selected from the group consisting of —H, C₁₋₄ alkoxy, C₁₋₆ alkyl, C₃₋₇ cycloalkyl, aryl, arylalkyl and heteroaryl.

Any bond represented by a dashed and solid line represents a bond selected from the group consisting of a single bond and a double bond.

Some embodiments provide a compound having the structure of Formula III or IV:

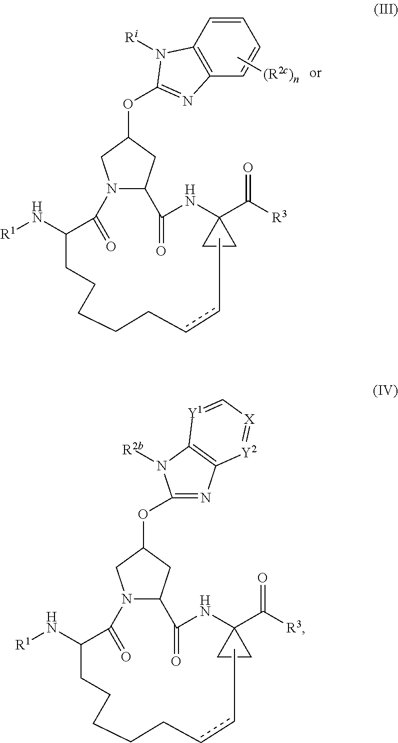

or a pharmaceutically acceptable salt or prodrug thereof wherein R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NHC(O)NR^{1a}R^{1b}, —C(O)OR^{1c}, and heteroaryl.

R^{1e} is selected from the group consisting of t-butyl, cycloalkyl, and heterocyclyl.

R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl

R^{1c} and R^{1d} are each separately selected from the group consisting of —H, C₁₋₄ alkoxy, C₁₋₆ alkyl, C₃₋₇ cycloalkyl, aryl, arylalkyl and heteroaryl.

X, Y, Y¹, and Y² are each independently selected from —CH— or —N—, wherein X and Y are not both —CH—, and X, Y¹, and Y² are not all —CH—;

R^{2b} is selected from the group consisting of C₁₋₆ alkyl optionally substituted with up to 5 fluoro, C₂₋₆ alkenyl, C₃₋₇ cycloalkyl, arylalkyl, optionally substituted aryl and optionally substituted heteroaryl.

Each R^{2c} is independently selected from the group consisting of halo, —C(O)OR^{1c}, —C(O)NR′R″, —NR′R″, —NHC(O)NR′R″, —NHC(O)OR^{1c}, —NHS(O)₂R^{1c}, C₂₋₆ alkyl, C₂₋₆ alkenyl, C₃₋₇ cycloalkyl, C₁₋₆ alkoxy, arylalkyl, polycyclic moiety, aryl, and heteroaryl, said C₂₋₆ alkyl, C₂₋₆ alkenyl, C₃₋₇ cycloalkyl, C₁₋₆ alkoxy, arylalkyl, polycyclic moiety, aryl, and heteroaryl each optionally substituted with one or more R¹². Each R¹² is independently selected from the group consisting of C₁₋₆ alkyl, C₃₋₇ cycloalkyl, C₁₋₆ alkoxy, heteroaryl, arylalkyl, aryl, —F (fluoro), —Cl (Chloro), —CN, —CF₃, —OCF₃, —C(O)NR′R″ and —NR′R″, wherein said C₂₋₆ alkyl, C₃₋₇ cycloalkyl, C₁₋₆ alkoxy, heteroaryl, arylalkyl, cycloalkylalkyl, and aryl are each optionally substituted with one or more R^{12a}. Each R^{12a} is independently selected from the group consisting of —F, —Cl, —CF₃, —OCF₃, C₁₋₆ alkyl, C₁₋₆ alkoxy, C₃₋₇ cycloalkyl, and aryl.

Each NR′R″ is separately selected wherein R′ and R″ are each independently selected from the group consisting of —H (hydrogen), halo, —C(O)NR′R″, optionally substituted C₁₋₆ alkyl, optionally substituted C₂₋₆ alkenyl, optionally substituted C₁₋₆ alkoxy, optionally substituted aryl, optionally substituted arylalkyl and optionally substituted heteroaryl; or R′ and R″ are taken together with the nitrogen to which they are attached to form heterocyclyl.

Rⁱ is C₁₋₆ alkyl optionally substituted with up to 5 fluoro.

R³ is —OH, —NHS(O)₂R^{3a}, —NHS(O)₂OR^{3a} or —NHS(O)₂NR^{3b}R^{3c}; where R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro.

Wherein R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{3b} and R^{3c} are taken together with the nitrogen to which they are attached to form a three- to six-membered heterocyclic ring, bonded to the parent structure through a nitrogen, and the heterocylic ring is optionally substituted with one or more substituents each independently selected from the group consisting of halo, cyano, nitro, C₁₋₆ alkyl, C₁₋₆ alkoxy, and phenyl.

Each t is independently 0, 1 or 2; and each q is independently 0, 1 or 2. Any bond represented by a dashed and solid line represents a bond selected from the group consisting of a single bond and a double bond.

Some embodiments provide a compound having the structure of Formula V:

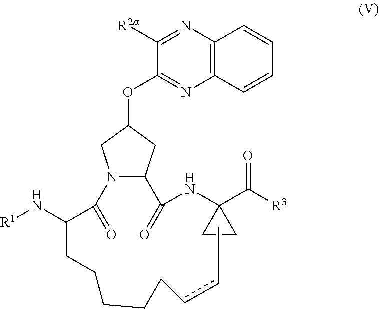

or a pharmaceutically acceptable salt or prodrug thereof wherein R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NCH(O)NR^{1a}R^{1b}, —C(O)OR^{1c}, and heteroaryl.

R^{1e} is selected from the group consisting of t-butyl, cycloalkyl, and heterocyclyl.

R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl.

R^{1c} and R^{1d} are each separately selected from the group consisting of —H, C₁₋₄ alkoxy, C₁₋₆ alkyl, C₃₋₇ cycloalkyl, aryl, arylalkyl and heteroaryl.

R^{2a} is selected from the group consisting of —H, —C(O)OR^{1c}, C₁₋₆ alkyl optionally substituted with up to 5 fluoro, C₂₋₆ alkenyl, C₃₋₇ cycloalkyl, optionally substituted aryl and optionally substituted heteroaryl.

R³ is —OH, —NHS(O)₂R^{3a}, —NHS(O)₂OR^{3a} or —NHS(O)₂NR^{3b}R^{3c}; where R^{1a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro.

Wherein R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{3b} and R^{3c} are taken together with the nitrogen to which they are attached to form a three- to six-membered heterocyclic ring, bonded to the parent structure through a nitrogen, and the heterocylic ring is optionally substituted with one or more substituents each independently selected from the group consisting of halo, cyano, nitro, C₁₋₆ alkyl, C₁₋₆ alkoxy, and phenyl.

Each t is independently 0, 1 or 2; and each q is independently 0, 1 or 2. Any bond represented by a dashed and solid line represents a bond selected from the group consisting of a single bond and a double bond.

Some embodiments provide a compound having the structure of Formulas VI-1 or VI-2:

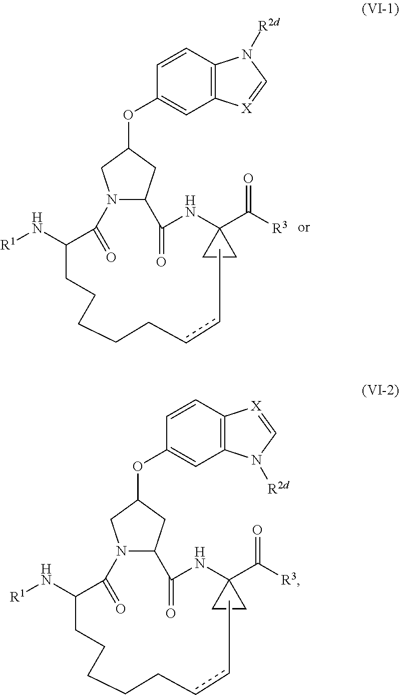

or a pharmaceutically acceptable salt or prodrug thereof, wherein R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NHC(O)NR^{1a}R^{1b}, —C(O)OR^{1c}, and heteroaryl.

R^{1e} is selected from the group consisting of t-butyl, cycloalkyl, and heterocyclyl.

R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl containing 1-3 heteroatoms independently selected from N and O; R^{1c} and R^{1d} are each separately selected from the group consisting of —H, C₁₋₄ alkoxy, C₁₋₆ alkyl, C₃₋₇ cycloalkyl, aryl, arylalkyl and heteroaryl.

X is —N— or —CH—, R^{2d} is selected from the group consisting of C₁₋₆ alkyl optionally substituted with up to 5 fluoro, C₂₋₆ alkenyl, C₃₋₇ cycloalkyl, arylalkyl, optionally substituted aryl and optionally substituted heteroaryl.

R³ is —OH, —NHS(O)₂R^{3a}, —NHS(O)₂OR^{3a} or —NHS(O)₂NR^{3b}R^{3c}; where R^{1a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro.

R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{3b} and R^{3c} are taken together with the nitrogen to which they are attached to form a three- to six-membered heterocyclic ring, bonded to the parent structure through a nitrogen, and the heterocylic ring is optionally substituted with one or more substituents each independently selected from the group consisting of halo, cyano, nitro, C₁₋₆ alkyl, C₁₋₆ alkoxy, and phenyl.

Each t is independently 0, 1 or 2; each q is independently 0, 1 or 2; and any bond represented by a dashed and solid line represents a bond selected from the group consisting of a single bond and a double bond.

Some embodiments provide a compound having the structure of Formula VIIa or VIIb:

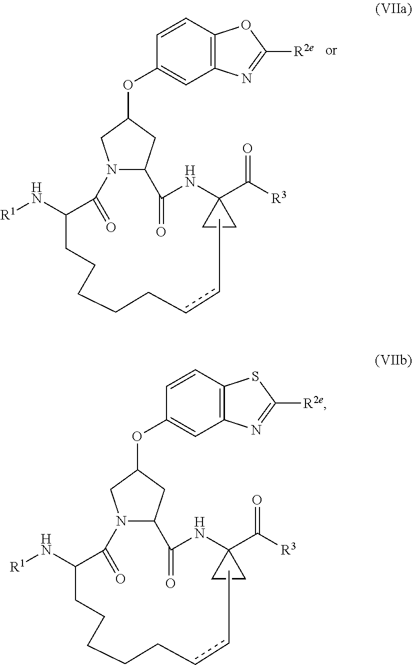

or a pharmaceutically acceptable salt or prodrug thereof wherein R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NCH(O)NR^{1a}R^{1b}, —C(O)OR^{1c}, and heteroaryl.

R^{1e} is selected from the group consisting of t-butyl, cycloalkyl, and heterocyclyl.

R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl containing 1-3 heteroatoms independently selected from N and O; R^{1c} and R^{1d} are each separately selected from the group consisting of —H, C₁₋₄ alkoxy, C₁₋₆ alkyl, C₃₋₇ cycloalkyl, aryl, arylalkyl and heteroaryl.

R^{2e} is selected from the group consisting of —H, —Br, —Cl, —C(O)OR^{1c}, —C(O)NR′R″, —NR′R″, —NHC(O)NR′R″, C₁₋₆ alkyl optionally substituted with up to 5 fluoro, C₂₋₆ alkenyl, C₃₋₇ cycloalkyl, optionally substituted C₁₋₆ alkoxy, optionally substituted aryl and optionally substituted heteroaryl; wherein R′ and R″ are each independently selected from the group consisting of —H, optionally substituted C₁₋₆ alkyl, optionally substituted C₂₋₆ alkenyl, optionally substituted aryl, optionally substituted arylalkyl and optionally substituted heteroaryl.

R³ is —OH, —NHS(O)₂R^{3a}, —NHS(O)₂OR^{3a} or —NHS(O)₂NR^{3b}R^{3c}; where R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro.

wherein R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{3b} and R^{3c} are taken together with the nitrogen to which they are attached to form a three- to six-membered heterocyclic ring, bonded to the parent structure through a nitrogen, and the heterocylic ring is optionally substituted with one or more substituents each independently selected from the group consisting of halo, cyano, nitro, C₁₋₆ alkyl, C₁₋₆ alkoxy, and phenyl.

Each t is independently 0, 1 or 2; each q is independently 0, 1 or 2; and any bond represented by a dashed and solid line represents a bond selected from the group consisting of a single bond and a double bond.

Some embodiments provide a compound having the structure of Formula VIIIa:

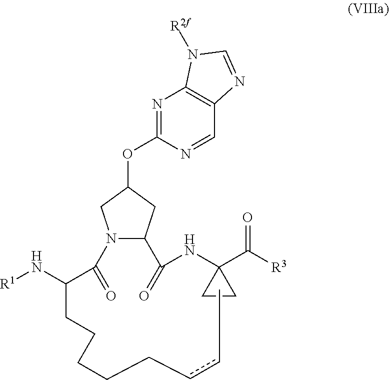

or a pharmaceutically acceptable salt or prodrug thereof wherein R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NCH(O)NR^{1a}R^{1b}, —C(O)OR^{1c}, and heteroaryl.

R^{1e} is selected from the group consisting of t-butyl, cycloalkyl, and heterocyclyl.

R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl containing 1-3 heteroatoms independently selected from N and O; R^{1c} and R^{1d} are each separately selected from the group consisting of —H, C₁₋₄ alkoxy, C₁₋₆ alkyl, C₃₋₇ cycloalkyl, aryl, arylalkyl and heteroaryl.

R^{2f} is selected from the group consisting of C₁₋₆ alkyl optionally substituted with up to 5 fluoro, C₂₋₆ alkenyl, C₃₋₇ cycloalkyl, arylalkyl, optionally substituted aryl and optionally substituted heteroaryl.

R³ is —OH, —NHS(O)₂R^{3a}, —NHS(O)₂OR^{3a} or —NHS(O)₂NR^{3b}R^{3c}; where R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro.

Wherein R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{3b} and R^{3c} are taken together with the nitrogen to which they are attached to form a three- to six-membered heterocyclic ring, bonded to the parent structure through a nitrogen, and the heterocylic ring is optionally substituted with one or more substituents each independently selected from the group consisting of halo, cyano, nitro, C₁₋₆ alkyl, C₁₋₆ alkoxy, and phenyl.

Each t is independently 0, 1 or 2; each q is independently 0, 1 or 2; and any bond represented by a dashed and solid line represents a bond selected from the group consisting of a single bond and a double bond.

Some embodiments provide a compound having the structure of Formula IX:

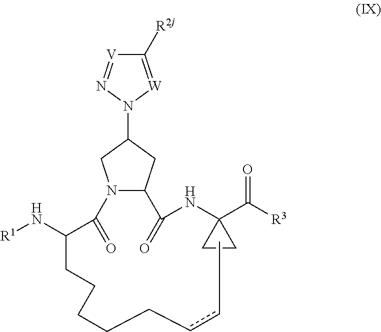

or a pharmaceutically acceptable salt or prodrug thereof wherein R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NCH(O)NR^{1a}R^{1b}, —C(O)OR^{1c}, and heteroaryl.

R^{1e} is selected from the group consisting of t-butyl, cycloalkyl, and heterocyclyl.

R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl containing 1-3 heteroatoms independently selected from N and O; R^{1c} and R^{1d} are each separately selected from the group consisting of —H, C₁₋₄ alkoxy, C₁₋₆ alkyl, C₃₋₇ cycloalkyl, aryl, arylalkyl and heteroaryl.

R^{2g} is selected from the group consisting of —H, —Br, —Cl, —C(O)OR^{1c}, —C(O)NR′R″, —NR′R″, —NHC(O)NR′R″, C₁₋₆ alkyl optionally substituted with up to 5 fluoro, C₂₋₆ alkenyl, C₃₋₇ cycloalkyl, optionally substituted C₁₋₆ alkoxy, optionally substituted aryl and optionally substituted heteroaryl; wherein R′ and R″ are each independently selected from the group consisting of —H, optionally substituted C₁₋₆ alkyl, optionally substituted C₂₋₆ alkenyl, optionally substituted aryl, optionally substituted arylalkyl and optionally substituted heteroaryl.

R³ is —OH, —NHS(O)₂R^{3a}, —NHS(O)₂OR^{3a} or —NHS(O)₂NR^{3b}R^{3c}; where R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro.

Wherein R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{3b} and R^{3c} are taken together with the nitrogen to which they are attached to form a three- to six-membered heterocyclic ring, bonded to the parent structure through a nitrogen, and the heterocylic ring is optionally substituted with one or more substituents each independently selected from the group consisting of halo, cyano, nitro, C₁₋₆ alkyl, C₁₋₆ alkoxy, and phenyl.

Each t is independently 0, 1 or 2; each q is independently 0, 1 or 2; and any bond represented by a dashed and solid line represents a bond selected from the group consisting of a single bond and a double bond.

Some embodiments provide a compound having the structure of Formula X:

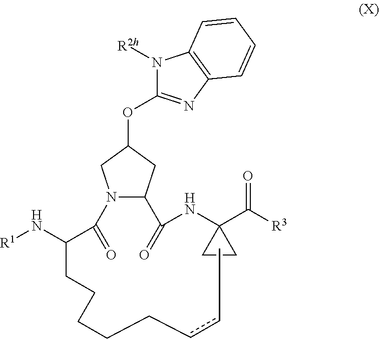

or a pharmaceutically acceptable salt or prodrug thereof wherein R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NHC(O)NR^{1a}R^{1b}, —C(O)OR^{1c}, and heteroaryl.

R^{1e} is selected from the group consisting of t-butyl, cycloalkyl, and heterocyclyl.

R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl containing 1-3 heteroatoms independently selected from N and O; R^{1c} and R^{1d} are each separately selected from the group consisting of —H, C₁₋₆ alkyl, C₃₋₇ cycloalkyl, aryl, arylalkyl and heteroaryl.

R^{2h} is selected from the group consisting of n-propyl, cyclopropyl, n-butyl, t-butyl, 1-sec-butyl and phenyl.

R³ is —OH, —NHS(O)₂R^{3a}, —NHS(O)₂OR^{3a} or —NHS(O)₂NR^{3b}R^{3c}; where R^{1a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro.

Wherein R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{3b} and R^{3c} are taken together with the nitrogen to which they are attached to form a three- to six-membered heterocyclic ring, bonded to the parent structure through a nitrogen, and the heterocylic ring is optionally substituted with one or more substituents each independently selected from the group consisting of halo, cyano, nitro, C₁₋₆ alkyl, C₁₋₆ alkoxy, and phenyl.

Each t is independently 0, 1 or 2; each q is independently 0, 1 or 2; and any bond represented by a dashed and solid line represents a bond selected from the group consisting of a single bond and a double bond.

Some embodiments provide a pharmaceutical composition comprising a pharmaceutically acceptable excipient and a compound of any one of Formulas I, Ia, II, III, IV, V, VI-1, VI-2, VII, VIII, IX, X, XI, and XII, or any compounds disclosed herein.

Some embodiments provide a method of inhibiting NS3/NS4 protease activity comprising contacting a NS3/NS4 protease with a compound of any one of Formulas I, Ia, II, III, IV, V, VI-1, VI-2, VII, VIII, IX, X, XI, and XII, any compounds disclosed herein, or a pharmaceutical composition disclosed herein.

Some embodiments provide a method of treating liver fibrosis in an individual, the method comprising administering to the individual an effective amount of a compound of any one of Formulas I, Ia, II, III, IV, V, VI-1, VI-2, VII, VIII, IX, X, XI, and XII, any compounds disclosed herein, or a pharmaceutical composition disclosed herein.

Some embodiments provide a method of increasing liver function in an individual having a hepatitis C virus infection, the method comprising administering to the individual an effective amount of a compound of any one of Formulas I, Ia, II, III, IV, V, VI-1, VI-2, VII, VIII, IX, X, XI, and XII, any compounds disclosed herein, or a pharmaceutical composition disclosed herein.

### DETAILED DESCRIPTION OF THE PREFERRED EMBODIMENT

### Definitions

As used herein, common organic abbreviations are defined as follows:
Ac Acetyl
Ac₂O Acetic anhydride
aq. Aqueous
Bn Benzyl
Bz Benzoyl
BOC or Boc tert-Butoxycarbonyl
Bu n-Butyl
cat. Catalytic
Cbz Carbobenzyloxy
CDI 1,1′-carbonyldiimidazole
Cy (c-C₆H₁₁) Cyclohexyl
° C. Temperature in degrees Centigrade
DBU 1,8-Diazabicyclo[5.4.0]undec-7-ene
DCE 1,2-Dichloroethane
DCM methylene chloride
DIEA Diisopropylethylamine
DMA Dimethylacetamide
DMAP 4-(Dimethylamino)pyridine
DME Dimethoxyethane
DMF N,N′-Dimethylformamide
DMSO Dimethylsulfoxide
Et Ethyl
EtOAc Ethyl acetate
g Gram(s)
h Hour (hours)
HATU 2-(1H-7-azabenzotriazol-1-yl)-1,1,3,3-tetramethyl uronium hexafluorophosphate
HOBT 1-Hydroxybenzotriazole
HPLC High performance liquid chromatography
iPr Isopropyl
IU International Units
LCMS Liquid chromatography-mass spectrometry
LDA Lithium diisopropylamide
mCPBA meta-Chloroperoxybenzoic Acid
MeOH Methanol
MeCN Acetonitrile
mL Milliliter(s)
MTBE Methyl tertiary-butyl ether
NH₄OAc Ammonium acetate
PG Protecting group
Pd/C Palladium on activated carbon
ppt Precipitate
PyBOP (Benzotriazol-1-yloxy)tripyrrolidinophosphonium hexafluorophosphate
RCM Ring closing metathesis
rt Room temperature
sBuLi sec-Butylithium
TEA Triethylamine
TCDI 1,1′-Thiocarbonyl diimidazole
Tert, t tertiary
TFA Trifluoracetic acid
THE Tetrahydrofuran
TLC Thin-layer chromatography
TMEDA Tetramethylethylenediamine
μL Microliter(s)

As used herein, the term “hepatic fibrosis,” used interchangeably herein with “liver fibrosis,” refers to the growth of scar tissue in the liver that can occur in the context of a chronic hepatitis infection.

The terms “individual,” “host,” “subject,” and “patient” are used interchangeably herein, and refer to a mammal, including, but not limited to, primates, including simians and humans.

As used herein, the term “liver function” refers to a normal function of the liver, including, but not limited to, a synthetic function, including, but not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

The term “sustained viral response” (SVR; also referred to as a “sustained response” or a “durable response”), as used herein, refers to the response of an individual to a treatment regimen for HCV infection, in terms of serum HCV titer. Generally, a “sustained viral response” refers to no detectable HCV RNA (e.g., less than about 500, less than about 200, or less than about 100 genome copies per milliliter serum) found in the patient's serum for a period of at least about one month, at least about two months, at least about three months, at least about four months, at least about five months, or at least about six months following cessation of treatment.

“Treatment failure patients” as used herein generally refers to HCV-infected patients who failed to respond to previous therapy for HCV (referred to as “non-responders”) or who initially responded to previous therapy, but in whom the therapeutic response was not maintained (referred to as “relapsers”). The previous therapy generally can include treatment with IFN-α monotherapy or IFN-α combination therapy, where the combination therapy may include administration of IFN-α and an antiviral agent such as ribavirin.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “host,” “subject,” and “patient” are used interchangeably herein, and refer to a mammal, including, but not limited to, murines, simians, humans, mammalian farm animals, mammalian sport animals, and mammalian pets.

As used herein, the term “a Type I interferon receptor agonist” refers to any naturally occurring or non-naturally occurring ligand of human Type I interferon receptor, which binds to and causes signal transduction via the receptor. Type I interferon receptor agonists include interferons, including naturally-occurring interferons, modified interferons, synthetic interferons, pegylated interferons, fusion proteins comprising an interferon and a heterologous protein, shuffled interferons; antibody specific for an interferon receptor; non-peptide chemical agonists; and the like.

As used herein, the term “Type II interferon receptor agonist” refers to any naturally occurring or non-naturally occurring ligand of human Type II interferon receptor that binds to and causes signal transduction via the receptor. Type II interferon receptor agonists include native human interferon-γ, recombinant IFN-γ species, glycosylated IFN-γ species, pegylated IFN-γ species, modified or variant IFN-γ species, IFN-γ fusion proteins, antibody agonists specific for the receptor, non-peptide agonists, and the like.

As used herein, the term “a Type III interferon receptor agonist” refers to any naturally occurring or non-naturally occurring ligand of human IL-28 receptor α (“IL-28R”), the amino acid sequence of which is described by Sheppard, et al., infra., that binds to and causes signal transduction via the receptor.

As used herein, the term “interferon receptor agonist” refers to any Type I interferon receptor agonist, Type II interferon receptor agonist, or Type III interferon receptor agonist.

The term “dosing event” as used herein refers to administration of an antiviral agent to a patient in need thereof, which event may encompass one or more releases of an antiviral agent from a drug dispensing device. Thus, the term “dosing event,” as used herein, includes, but is not limited to, installation of a continuous delivery device (e.g., a pump or other controlled release injectible system); and a single subcutaneous injection followed by installation of a continuous delivery system.

“Continuous delivery” as used herein (e.g., in the context of “continuous delivery of a substance to a tissue”) is meant to refer to movement of drug to a delivery site, e.g., into a tissue in a fashion that provides for delivery of a desired amount of substance into the tissue over a selected period of time, where about the same quantity of drug is received by the patient each minute during the selected period of time.

By “substantially continuous” as used in, for example, the context of “substantially continuous infusion” or “substantially continuous delivery” is meant to refer to delivery of drug in a manner that is substantially uninterrupted for a pre-selected period of drug delivery, where the quantity of drug received by the patient during any 8 hour interval in the pre-selected period never falls to zero. Furthermore, “substantially continuous” drug delivery can also encompass delivery of drug at a substantially constant, pre-selected rate or range of rates (e.g., amount of drug per unit time, or volume of drug formulation for a unit time) that is substantially uninterrupted for a pre-selected period of drug delivery.

By “substantially steady state” as used in the context of a biological parameter that may vary as a function of time, it is meant that the biological parameter exhibits a substantially constant value over a time course, such that the area under the curve defined by the value of the biological parameter as a function of time for any 8 hour period during the time course (AUC8 hr) is no more than about 20% above or about 20% below, and preferably no more than about 15% above or about 15% below, and more preferably no more than about 10% above or about 10% below, the average area under the curve of the biological parameter over an 8 hour period during the time course (AUC8 hr average). The AUC8 hr average is defined as the quotient (q) of the area under the curve of the biological parameter over the entirety of the time course (AUCtotal) divided by the number of 8 hour intervals in the time course (total/3 days), i.e., q=(AUCtotal)/(total/3 days). For example, in the context of a serum concentration of a drug, the serum concentration of the drug is maintained at a substantially steady state during a time course when the area under the curve of serum concentration of the drug over time for any 8 hour period during the time course (AUC8 hr) is no more than about 20% above or about 20% below the average area under the curve of serum concentration of the drug over an 8 hour period in the time course (AUC8 hr average), i.e., the AUC8 hr is no more than 20% above or 20% below the AUC8 hr average for the serum concentration of the drug over the time course.

As used herein, “hydrogen bond” refers to an attractive force between an electronegative atom (such as oxygen, nitrogen, sulfur or halogen) and a hydrogen atom which is linked covalently to another electronegative atom (such as oxygen, nitrogen, sulfur or halogen). See, e.g., Stryer et. al. “Biochemistry”, Fifth Edition 2002, Freeman & Co. N.Y. Typically, the hydrogen bond is between a hydrogen atom and two unshared electrons of another atom. A hydrogen bond may be present when the distance between the electronegative atom to which the hydrogen is covalently bonded, and the other electronegative atom to which the hydrogen is attracted, is 2.2 angstroms to about 3.8 angstroms, and the angle formed by the three atoms (electronegative atom covalently bound to hydrogen, hydrogen, and electronegative atom not-covalently bound) deviates from 180 degrees by about 60 degrees or less. The distance between the two electronegative atoms may be referred to herein as the “hydrogen bond length,” and the angle formed by the three atoms (electronegative atom covalently bound to hydrogen, hydrogen, and electronegative atom not-covalently bound) may be referred to herein as the “hydrogen bond angle”, as shown in Figure X:

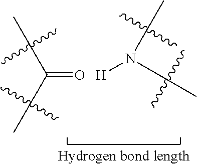

In some instances, stronger hydrogen bonds are formed when the hydrogen bond length is shorter; thus, in some instances, hydrogen bond lengths may range from about 2.4 angstroms to about 3.6 angstroms, or about 2.5 angstroms to about 3.4 angstroms. In some instances, stronger hydrogen bonds are formed when the hydrogen bond angle is closer to being linear; thus, in some instances, hydrogen bond angles may deviate from 180 degrees by about 25 degrees or less, or by about 10 degrees or less.

As used herein, “non-polar interaction” refers to the proximity of a non-polar atom, molecule or moiety to another atom, molecule or moiety, or the proximity of an atom, molecule or moiety with low polarity to another atom, molecule or moiety, sufficient for van der Waals interaction between the atoms/molecules. See, e.g., Stryer et. al. “Biochemistry”, Fifth Edition 2002, Freeman & Co. N.Y. Typically, the distance between heavy (non-hydrogen) atoms of non-polar interacting moieties is sufficiently close to exclude polar solvent molecules, such as water molecules. Non-polar interactions may range from about 2.5 angstroms to about 4.8 angstroms, from about 2.5 angstroms to about 4.3 angstroms, or from about 2.5 angstroms to about 3.8 angstroms. As used herein a non-polar moiety or moiety with low polarity refers to moieties with low dipolar moments (typically dipolar moments less than the dipolar moment of O—H bonds of H₂O and N—H bonds of NH₃), and/or moieties that are not typically present in hydrogen bonding or electrostatic interactions. Examples of moieties with low polarity are alkyl, alkenyl, and unsubstituted aryl moieties. In some embodiments, the term “non-polar interactions” refers to “hydrophobic interactions” and/or “van der Waals Interactions.”

As used herein, an NS3 protease S1′ pocket moiety refers to a moiety of the NS3 protease that interacts with the amino acid positioned one residue C-terminal to the cleavage site of the substrate polypeptide cleaved by NS3 protease as described in paragraph [0066] of WO 2007/015824 incorporated herein in its entirety. Exemplary moieties include, but are not limited to, atoms of the peptide backbone or side chains of amino acids Lys136, Gly137, Ser139, His57, Gly58, Gln41, Ser42, and Phe43, see Yao. et. al., Structure 1999, 7, 1353, incorporated herein in its entirety.

As used herein, an NS3 protease S2 pocket moiety refers to a moiety of the NS3 protease that interacts with the amino acid positioned two residues N-terminal to the cleavage site of the substrate polypeptide cleaved by NS3 protease as described in paragraph [0067] of WO 2007/015824, incorporated herein in its entirety. Exemplary moieties include, but are not limited to, atoms of the peptide backbone or side chains of amino acids Tyr56, Gly58, Ala59, Gly60, Gln41, His57, Va178, Asp79, Gln80 and Asp81, see Yao. et. al., Structure 1999, 7, 1353.

The term “alkyl” as used herein refers to a radical of a fully saturated hydrocarbon, including, but not limited to, methyl, ethyl, n-propyl, isopropyl (or i-propyl), n-butyl, isobutyl, tert-butyl (or t-butyl), n-hexyl,

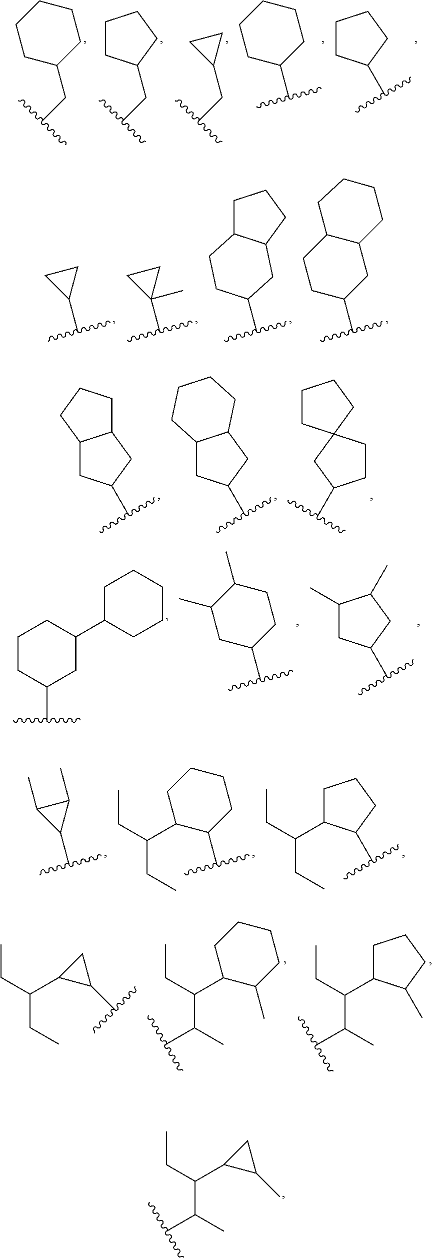

and the like. For example, the term “alkyl” as used herein includes radicals of fully saturated hydrocarbons defined by the following general formula's: the general formula for linear or branched fully saturated hydrocarbons not containing a cyclic structure is CₙH₂ₙ₊₂; the general formula for a fully saturated hydrocarbon containing one ring is CₙH₂ₙ; the general formula for a fully saturated hydrocarbon containing two rings is CₙH₂₍ₙ₋₁₎; the general formula for a saturated hydrocarbon containing three rings is CₙH₂₍ₙ₋₂₎. When a more specific term for alkyl (such as propyl, butyl, etc.) is used without specifying linear or branched, the term is to be interpreted to include linear and branched alkyl.

The term “halo” used herein refers to fluoro, chloro, bromo, or iodo.

The term “alkoxy” used herein refers to straight or branched chain alkyl radical covalently bonded to the parent molecule through an —O— linkage. Examples of alkoxy groups include, but are not limited to, methoxy, ethoxy, propoxy, isopropoxy, butoxy, n-butoxy, sec-butoxy, t-butoxy and the like. When a more specific term for alkoxy (such as propoxy, butaoxy, etc.) is used without specifying linear or branched, the term is to be interpreted to include linear and branched alkoxy.

The term “alkenyl” used herein refers to a monovalent straight or branched chain radical of from two to twenty carbon atoms containing a carbon double bond including, but not limited to, 1-propenyl, 2-propenyl, 2-methyl-1-propenyl, 1-butenyl, 2-butenyl, and the like.

The term “alkynyl” used herein refers to a monovalent straight or branched chain radical of from two to twenty carbon atoms containing a carbon triple bond including, but not limited to, 1-propynyl, 1-butynyl, 2-butynyl, and the like.

The term “polycyclic moiety” used herein refers a bicyclic moiety or tricyclic moiety optionally containing one or more heteroatoms wherein at least one of the rings is an aryl or heteroaryl ring and at least one of the rings is not an aryl or heteroaryl ring. The bicyclic moiety contains two rings wherein the rings are fused. The bicyclic moiety can be appended at any position of the two rings. For example, bicyclic moiety may refer to a radical including but not limited to:

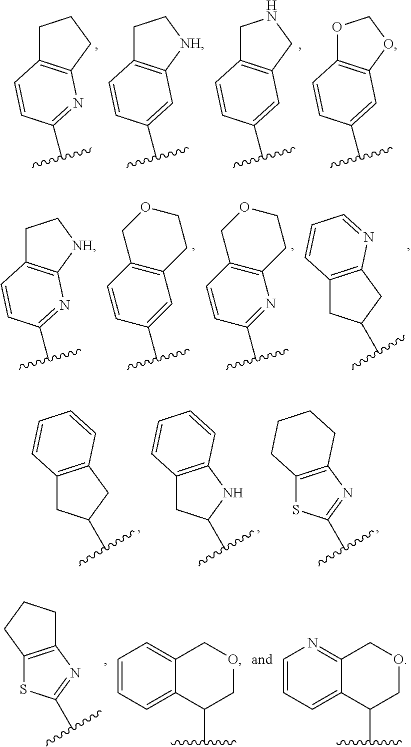

The tricyclic moiety contains a bicyclic moiety with an additional fused ring. The tricyclic moiety can be appended at any position of the three rings. For example, tricyclic moiety may refer to a radical including but not limited to:

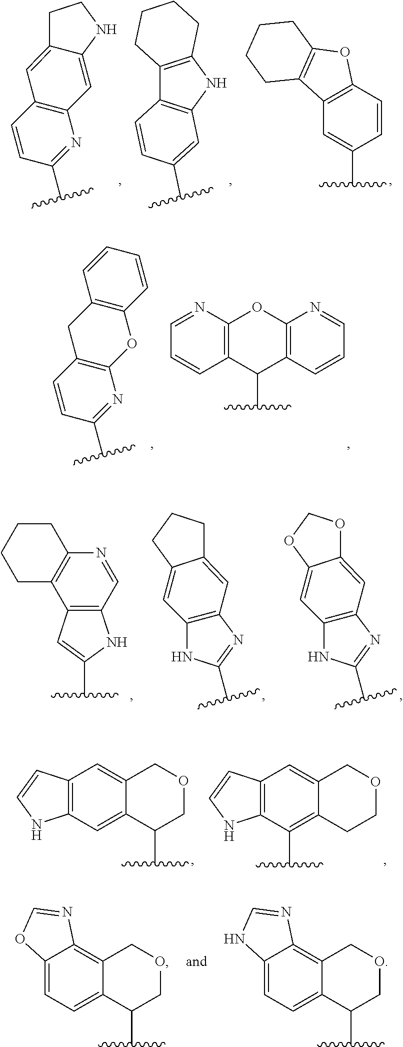

The term “aryl” used herein refers to homocyclic aromatic radical whether one ring or multiple fused rings. Examples of aryl groups include, but are not limited to, phenyl, naphthyl, phenanthrenyl, naphthacenyl, and the like.

The term “cycloalkyl” used herein refers to saturated aliphatic ring system radical having three to twenty carbon atoms including, but not limited to, cyclopropyl, cyclopentyl, cyclohexyl, cycloheptyl, and the like.

The term “cycloalkenyl” used herein refers to aliphatic ring system radical having three to twenty carbon atoms having at least one carbon-carbon double bond in the ring. Examples of cycloalkenyl groups include, but are not limited to, cyclopropenyl, cyclopentenyl, cyclohexenyl, cycloheptenyl, bicyclo[3.1.0]hexyl, and the like.

The term “heterocyclic” or “heterocyclyl” or “heterocycloalkyl” used herein refers to cyclic non-aromatic ring system radical having at least one ring in which one or more ring atoms are not carbon, namely heteroatom. In fused ring systems, the one or more heteroatoms may be present in only one of the rings. Examples of heterocyclic groups include, but are not limited to, morpholinyl, tetrahydrofuranyl, dioxolanyl, pyrrolidinyl, pyranyl, piperidyl, piperazyl, oxetanyl and the like.

The term “heteroaryl” used herein refers to an aromatic group comprising one or more heteroatoms, whether one ring or multiple fused rings. When two or more heteroatoms are present, they may be the same or different. In fused ring systems, the one or more heteroatoms may be present in only one of the rings. Examples of heteroaryl groups include, but are not limited to, benzothiazyl, benzoxazyl, quinazolinyl, quinolinyl, isoquinolinyl, quinoxalinyl, pyridinyl, pyrrolyl, oxazolyl, indolyl, thiazyl and the like.

The term “heteroatom” used herein refers to S (sulfur), N (nitrogen), and O (oxygen).

The term “arylalkyl” used herein refers to one or more aryl groups appended to an alkyl radical. Examples of arylalkyl groups include, but are not limited to, benzyl, phenethyl, phenpropyl, phenbutyl, and the like.

The term “cycloalkylalkyl” used herein refers to one or more cycloalkyl groups appended to an alkyl radical. Examples of cycloalkylalkyl include, but are not limited to, cyclohexylmethyl, cyclohexylethyl, cyclopentylmethyl, cyclopentylethyl, and the like.

The term “heteroarylalkyl” used herein refers to one or more heteroaryl groups appended to an alkyl radical. Examples of heteroarylalkyl include, but are not limited to, pyridylmethyl, furanylmethyl, thiopheneylethyl, and the like.

The term “aryloxy” used herein refers to an aryl radical covalently bonded to the parent molecule through an —O— linkage.

The term “alkylthio” used herein refers to straight or branched chain alkyl radical covalently bonded to the parent molecule through an —S— linkage. Examples of alkoxy groups include, but are not limited to, methoxy, ethoxy, propoxy, isopropoxy, butoxy, n-butoxy, sec-butoxy, t-butoxy and the like.

The term “arylthio” used herein refers to an aryl radical covalently bonded to the parent molecule through an —S— linkage.

The term “alkylamino” used herein refers to nitrogen radical with one or more alkyl groups attached thereto. Thus, monoalkylamino refers to nitrogen radical with one alkyl group attached thereto and dialkylamino refers to nitrogen radical with two alkyl groups attached thereto.

The term “cyanoamino” used herein refers to nitrogen radical with nitrile group attached thereto.

The term “hydroxyalkyl” used herein refers to one or more hydroxy groups appended to an alkyl radical.

The term “aminoalkyl” used herein refers to one or more amino groups appended to an alkyl radical.

The term “arylalkyl” used herein refers to one or more aryl groups appended to an alkyl radical.

The term “carbamyl” used herein refers to RNHC(O)O—.

The term “keto” and “carbonyl” used herein refers to C═O.

The term “carboxy” used herein refers to —COOH.

The term “sulfamyl” used herein refers to —SO₂NH₂.

The term “sulfonyl” used herein refers to —SO₂—.

The term “sulfinyl” used herein refers to —SO—.

The term “thiocarbonyl” used herein refers to C═S.

The term “thiocarboxy” used herein refers to CSOH.

As used herein, a radical indicates species with one or more, unpaired electron such that the species containing the radical can be covalently bonded to one or more other species. Hence, in this context, a radical is not necessarily a free radical. Rather, a radical indicates a specific portion of a larger molecule. The term “radical” can be used interchangeably with the terms “group” and “moiety.”

As used herein, a substituted group is derived from the unsubstituted parent structure in which there has been an exchange of one or more hydrogen atoms for another atom or group. Unless otherwise indicated, when substituted, the substituent group(s) is (are) one or more group(s) individually and independently selected from C₁-C₆ alkyl, C₁-C₆ alkenyl, C₁-C₆ alkynyl, C₃-C₇ cycloalkyl (optionally substituted with halo, alkyl, alkoxy, carboxyl, CN, —SO₂-alkyl, —CF₃, and —OCF₃), C₃-C₆ heterocycloalkyl (e.g., tetrahydrofuryl) (optionally substituted with halo, alkyl, alkoxy, carboxyl, CN, —SO₂-alkyl, —CF₃, and —OCF₃), aryl (optionally substituted with halo, alkyl, alkoxy, carboxyl, CN, —SO₂-alkyl, —CF₃, and —OCF₃), heteroaryl (optionally substituted with halo, alkyl, alkoxy, carboxyl, CN, —SO₂-alkyl, —CF₃, and —OCF₃), halo (e.g., chloro, bromo, iodo and fluoro), cyano, hydroxy, C₁-C₆ alkoxy, aryloxy, sulfhydryl (mercapto), C₁-C₆ alkylthio, arylthio, mono- and di-(C₁-C₆)alkyl amino, quaternary ammonium salts, amino(C₁-C₆)alkoxy, hydroxy(C₁-C₆)alkylamino, amino(C₁-C₆)alkylthio, cyanoamino, nitro, carbamyl, keto (oxo), carbonyl, carboxy, glycolyl, glycyl, hydrazino, guanyl, sulfamyl, sulfonyl, sulfinyl, thiocarbonyl, thiocarboxy, and combinations thereof. The protecting groups that can form the protective derivatives of the above substituents are known to those of skill in the art and can be found in references such as Greene and Wuts Protective Groups in Organic Synthesis; John Wiley and Sons: New York, 1999. Wherever a substituent is described as “optionally substituted” that substituent can be substituted with the above substituents unless the context clearly dictates otherwise.

Asymmetric carbon atoms may be present in the compounds described. All such isomers, including diastereomers and enantiomers, as well as the mixtures thereof are intended to be included in the scope of the recited compound. In certain cases, compounds can exist in tautomeric forms. All tautomeric forms are intended to be included in the scope. Likewise, when compounds contain an alkenyl or alkenylene group, there exists the possibility of cis- and trans-isomeric forms of the compounds. Both cis- and trans-isomers, as well as the mixtures of cis- and trans-isomers, are contemplated. Thus, reference herein to a compound includes all of the aforementioned isomeric forms unless the context clearly dictates otherwise.

Isotopes may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

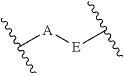

includes the substituent being oriented such that the A is attached at the leftmost attachment point of the molecule as well as attached at the rightmost attachment point of the molecule.

It is to be understood that certain radical naming conventions can include either a mono-radical or a di-radical, depending on the context. For example, where a substituent requires two points of attachment to the rest of the molecule, it is understood that the substituent is a di-radical. A substituent identified as alkyl, that requires two points of attachment, includes di-radicals such as —CH₂—, —CH₂CH₂—, —CH₂CH(CH₃)CH₂—, and the like; a substituent depicted as alkoxy that requires two points of attachment, includes di-radicals such as —OCH₂—, —OCH₂CH₂—, —OCH₂CH(CH₃)CH₂—, and the like: and a substituent depicted as arylC(O)— that requires two points of attachment, includes di-radicals such as

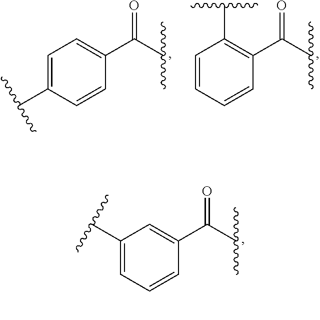

and the like.

Various forms are included in the embodiments, including polymorphs, solvates, hydrates, conformers, salts, and prodrug derivatives. A polymorph is a composition having the same chemical formula, but a different structure. A solvate is a composition formed by solvation (the combination of solvent molecules with molecules or ions of the solute). A hydrate is a compound formed by an incorporation of water. A conformer is a structure that is a conformational isomer. Conformational isomerism is the phenomenon of molecules with the same structural formula but different conformations (conformers) of atoms about a rotating bond. Salts of compounds can be prepared by methods known to those skilled in the art. For example, salts of compounds can be prepared by reacting the appropriate base or acid with a stoichiometric equivalent of the compound. A prodrug is a compound that undergoes biotransformation (chemical conversion) before exhibiting its pharmacological effects. For example, a prodrug can thus be viewed as a drug containing specialized protective groups used in a transient manner to alter or to eliminate undesirable properties in the parent molecule. Thus, reference herein to a compound includes all of the aforementioned forms unless the context clearly dictates otherwise.

Where a range of values is provided, it is understood that each intervening value, to the tenth of the unit of the lower limit unless the context clearly dictates otherwise, between the upper and lower limit of that range and any other stated or intervening value in that stated range is encompassed within the embodiments. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the embodiments.

Unless defined otherwise, all technical and scientific terms used herein have the same meaning as commonly understood by one of ordinary skill in the art to which the embodiments belong. Although any methods and materials similar or equivalent to those described herein can also be used in the practice or testing of the embodiments, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The present embodiments provide compounds of Formulae I, Ia, II, III, IV, V, VI-1, VI-2, VII, VIII, IX, X, XI, and XII, as well as pharmaceutical compositions and formulations comprising any compound of Formulae I, Ia, II, III, IV, V, VI-1, VI-2, VII, VIII, IX, X, XI, and XII. A subject compound is useful for treating HCV infection and other disorders, as discussed below.

### Formula I

The embodiments provide a compound having the structure of Formula I:

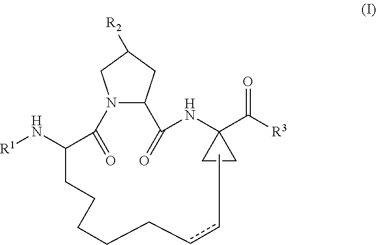

or a pharmaceutically acceptable salt or prodrug thereof, wherein R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NHC(O)NR^{1a}R^{1b}, —C(O)OR^{1c}. In some embodiments, said heteroaryl contains 1-3 heteroatoms independently selected from S, N, or O.

R^{1e} is selected from the group consisting of t-butyl, cycloalkyl, and heterocyclyl.

R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl, wherein in some embodiments, said heteroaryl may contain 1-3 heteroatoms independently selected from N or O.

R^{1c} and R^{1d} are each separately selected from the group consisting of —H, C₁₋₄ alkoxy, C₁₋₆ alkyl, C₃₋₇ cycloalkyl, aryl, arylalkyl and heteroaryl.

R² is selected from the group consisting of:

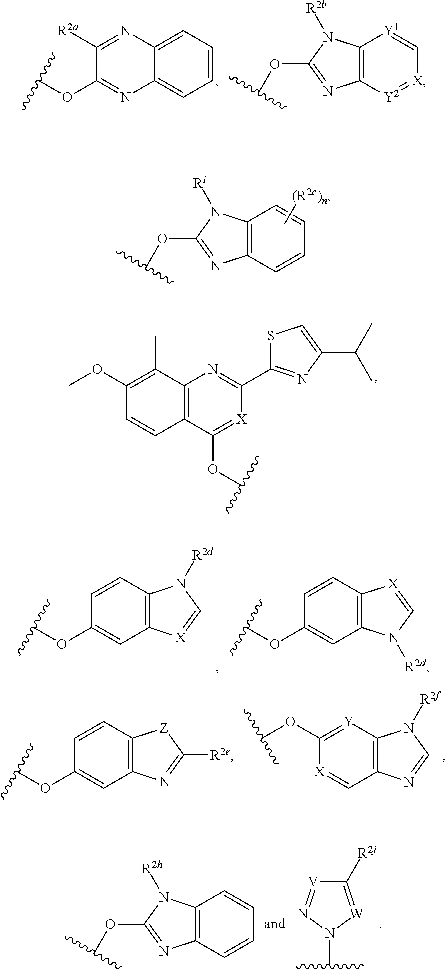

X, Y, Y¹, and Y² are each independently selected from —CH— or —N—, wherein X and Y are not both —CH—, and X, Y¹, and Y² are not all —CH—; Z is O or S; V and W are each independently selected from —CR^{2k}— or —N—, wherein V and W are not both —CR^{2k}—; n is 1, 2 or 3; and R^{2j} and R^{2k} are each independently selected from the group consisting of H, halo, optionally substituted aryl, optionally substituted heteroaryl; or R^{2j} and R^{2k} together form an aryl ring optionally substituted by 1-3 R^{2g}.

R^{2a}, R^{2e} and R^{2g} are each independently selected from the group consisting of halo, —C(O)OR^{1c}, —C(O)NR′R″, —NR′R″, —NHC(O)NR′R″, —NHC(O)OR^{1c}, —NHS(O)₂R^{1c}, C₁₋₆ alkyl optionally substituted with up to 5 fluoro, C₂₋₆ alkenyl, C₃₋₇ cycloalkyl, optionally substituted C₁₋₆ alkoxy, optionally substituted aryl and optionally substituted heteroaryl.

Each R^{2c} is independently selected from the group consisting of halo, —C(O)OR^{1c}, —C(O)NR′R″, —NR′R″, —NHC(O)NR′R″, —NHC(O)OR^{1c}, —NHS(O)₂R^{1c}, C₁₋₆ alkyl, C₂₋₆ alkenyl, C₃₋₇ cycloalkyl, C₁₋₆ alkoxy, arylalkyl, polycyclic moiety, aryl, and heteroaryl, wherein said C₁₋₆ alkyl, C₂₋₆ alkenyl, C₃₋₇ cycloalkyl, C₁₋₆ alkoxy, arylalkyl, polycyclic moiety, aryl, and heteroaryl each optionally substituted with one or more R¹². Each R¹² is independently selected from the group consisting of C₁₋₆ alkyl, C₃₋₇ cycloalkyl, C₁₋₆ alkoxy, heteroaryl, arylalkyl, aryl, —F (fluoro), —Cl (Chloro), —CN, —CF₃, —OCF₃, —C(O)NR′R″ and —NR′R″, wherein said C₁₋₆ alkyl, C₃₋₇ cycloalkyl, C₁₋₆ alkoxy, heteroaryl, arylalkyl, cycloalkylalkyl, and aryl are each optionally substituted with one or more R^{12a}. Each R^{12a} is independently selected from the group consisting of —F, —Cl, —CF₃, —OCF₃, C₁₋₆ alkyl, C₁₋₆ alkoxy, C₃₋₇ cycloalkyl, and aryl.

Each NR′R″ is separately selected wherein R′ and R″ are each independently selected from the group consisting of —H (hydrogen), halo, —C(O)NR′R″, optionally substituted C₁₋₆ alkyl, optionally substituted C₂₋₆ alkenyl, optionally substituted C₁₋₆ alkoxy, optionally substituted aryl, optionally substituted arylalkyl and optionally substituted heteroaryl; or R′ and R″ are taken together with the nitrogen to which they are attached to form heterocyclyl.

R^{2b}, R^{2d}, and R^{2f} are each independently selected from the group consisting of C₁₋₆ alkyl optionally substituted with up to 5 fluoro, C₂₋₆ alkenyl, C₃₋₇ cycloalkyl, arylalkyl, optionally substituted aryl and optionally substituted heteroaryl; R^{2h} is selected from the group consisting of propyl, butyl and phenyl; Rⁱ is C₁₋₆ alkyl optionally substituted with up to 5 fluoro.

R³ is —OH, —NHS(O)₂R^{3a}, —NHS(O)₂OR^{3a} or —NHS(O)₂NR^{3b}R^{3c}; where R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro.

R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{3b} and R^{3c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl; each t is independently 0, 1 or 2; and each q is independently 0, 1 or 2. Any bond represented by a dashed and solid line represents a bond selected from the group consisting of a single bond and a double bond.

Provided that if R² is

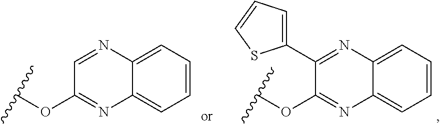

then R¹ is not phenyl; provided that if R² is

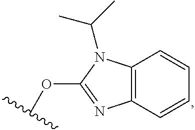

then R¹ is not —C(O)O-t-butyl, phenyl or phenyl substituted with one or more substituents selected from the group consisting of fluoro, chloro and —CF₃.

Provided that if R² is

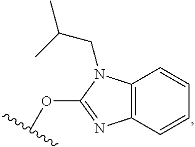

then R¹ is not —C(O)O-t-butyl or phenyl substituted with one or more substituents selected from the group consisting of fluoro and —CF₃.

Provided that if R² is

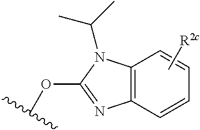

and R^{2c} is —F or methyl, then R¹ is not —C(O)O-t-butyl or phenyl.

Provided that if R² is

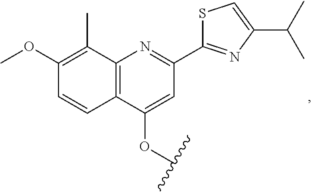

then R¹ is not —C(O)O-t-butyl, benzoxazyl, t-butylthiazyl, phenyl or phenyl substituted with one or more substituents selected from the group consisting of fluoro, chloro, methyl, —CF₃ and —OCF₃.

In some embodiments, compounds of Formula I have the structure of Formula Ia:

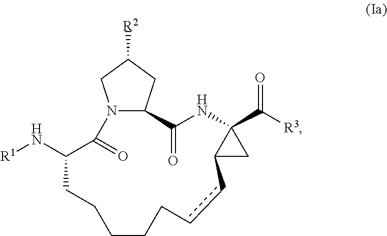

wherein R¹, R², and R³ are the same as defined above.

Some embodiments provide compounds of Formula I or Formula Ia, in which R¹ is selected from the group consisting of —C(O)O—R^{1e}, optionally substituted heteroaryl, and aryl optionally substituted with one or more substitutents each independently selected from the group consisting of C₁₋₆ alkyl, fluoro, amino, —CF₃, —OCF₃, —C(O)NR^{1a}R^{1b}, —NHC(O)NR^{1a}R^{1b}, —C(O)OH, and oxazolyl. In some embodiments, R^{1a} and R^{1b} are taken together with the nitrogen to which they are attached to form piperazinyl or morpholinyl, each optionally substituted with one or more substituents independently selected from C₁₋₆ alkyl, C₂₋₆ alkenyl, C₂₋₆ alkynyl, —C(O)OR^{1c}, —C(O)R^{1d}, hydroxy-C₁₋₆alkyl, amino-C₁₋₆alkyl, aryl-C₁₋₆alkyl, optionally substituted aryl, and heteroaryl, wherein in some embodiments, said heteroaryl may contain 1-3 heteroatoms independently selected from N or O; and R^{1c} and R^{1d} are each separately selected from the group consisting of —H, C₁₋₄ alkoxy, C₁₋₆ alkyl, C₃₋₇ cycloalkyl, aryl, arylalkyl and heteroaryl.

Some embodiments provide compounds of Formula I or Formula Ia, in which R¹ is aryl optionally substituted with one or more substitutents each independently selected from the group consisting of —C(O)NR^{1a}R^{1b} and —NHC(O)NR^{1a}R^{1b}, wherein R^{1a} and R^{1b} are taken together with the nitrogen to which they are attached to form piperazinyl or morpholinyl, each optionally substituted with C₁₋₆ alkyl, hydroxy-C₁₋₆alkyl, amino-C₁₋₆alkyl, aryl-C₁₋₆alkyl, —C(O)OR^{1c}, —C(O)R^{1d}, optionally substituted aryl, and heteroaryl, wherein in some embodiments, said heteroaryl may contain 1-3 heteroatoms independently selected from N or O. In some embodiments, R^{1a} and R^{1b} are taken together with the nitrogen to which they are attached to form:

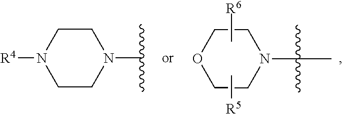

wherein R⁴ is selected from the group consisting of —H, C₁₋₆ alkyl optionally substituted with one or more amine, aryl or hydroxy, aryl optionally substituted with C₁₋₄ alkyl, —CF₃, or —OCF₃, and —C(O)R^{4a}, where R^{4a} is selected from the group consisting of C₁₋₄ alkoxy, C₃₋₇ cycloalkyl and aryl; and R⁵ and R⁶ are each independently —H or C₁₋₆ alkyl optionally substituted with phenyl.

Some embodiments provide compounds of Formula I or Formula Ia, in which R² is selected from the group consisting of

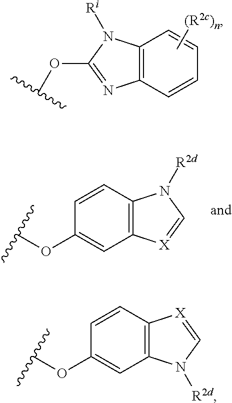

wherein each R^{2c} is independently selected from the group consisting of —CF₃, —Br, —Cl, —C(O)OH, —C(O)NR′R″, —NR′R″, —NHC(O)NR′R″, —NHC(O)OR^{1c}, —NHS(O)₂R^{1c}, C₁₋₆ alkyl, C₂₋₆ alkenyl, C₁₋₆ alkoxy, polycyclic moiety, phenyl, and heteroaryl, said C₁₋₆ alkyl, C₂₋₆ alkenyl, C₁₋₆ alkoxy, polycyclic moiety, aryl, and heteroaryl each optionally substituted with one or more R¹²; and in some embodiments, said heteroaryl may be selected from the group consisting of furanyl, thiazolyl, oxazolyl, thiophenyl, pyrazolyl, and benzothiazolyl.

Each R¹² is independently selected from the group consisting of C₁₋₆ alkyl, C₃₋₇ cycloalkyl, C₁₋₆ alkoxy, pyridinyl, phenylalkyl, phenyl, —F (fluoro), —Cl (Chloro), —CN, —CF₃, —OCF₃, —C(O)NR′R″, morpholinyl, pyrrolidinyl, piperidinyl, C₃₋₇ cycloalkyl-alkyl, wherein said C₁₋₆ alkyl, C₃₋₇ cycloalkyl, C₁₋₆ alkoxy, pyridinyl, phenylalkyl, phenyl, morpholinyl, pyrrolidinyl, piperidiny, are each optionally substituted with one or more R^{12a}.

Each NR′R″ is separately selected wherein R′ and R″ are each independently selected from the group consisting of —H (hydrogen), —F, —Cl, —C(O)NR′R″, C₁₋₆ alkyl, C₂₋₆ alkenyl, C₁₋₆ alkoxy, phenyl, phenylalkyl, and heteroaryl; and each R^{12a} is independently selected from the group consisting of —F, —C₁, C₁₋₆ alkyl, C₁₋₆ alkoxy, C₃₋₇ cycloalkyl, and aryl.

R^{2d} is selected from the group consisting of C₁₋₆ alkyl optionally substituted with up to 5 fluoro, C₃₋₇ cycloalkyl, arylalkyl, optionally substituted aryl and optionally substituted heteroaryl; and Rⁱ is ethyl or i-propyl.

Some embodiments provide compounds of Formula I or Formula Ia, in which R² is

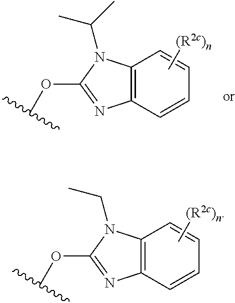

In some embodiments, each R^{2c} is independently selected from the group consisting of —CF₃, —Br, —Cl, —C(O)OH, —C(O)NR′R″, —NR′R″, —NHC(O)NR′R″, —NHC(O)OR^{1c}, —NHS(O)₂R^{1c}, C₁₋₆ alkyl, C₂₋₆ alkenyl, C₁₋₆ alkoxy, polycyclic moiety, phenyl, and heteroaryl, said C₁₋₆ alkyl, C₂₋₆ alkenyl, C₁₋₆ alkoxy, polycyclic moiety, aryl, and heteroaryl each optionally substituted with one or more R¹²; and in some embodiments, said heteroaryl may be selected from the group consisting of furanyl, thiazolyl, oxazolyl, thiophenyl, pyrazolyl, and benzothiazolyl.

In some embodiments, each R¹² is independently selected from the group consisting of C₁₋₆ alkyl, C₃₋₇ cycloalkyl, C₁₋₆ alkoxy, pyridinyl, phenylalkyl, phenyl, —F (fluoro), —Cl(Chloro), —CN, —CF₃, —OCF₃, —C(O)NR′R″ and morpholinyl, pyrrolidinyl, piperidiny, C₃₋₇ cycloalkyl-alkyl, wherein said C₁₋₆ alkyl, C₃₋₇ cycloalkyl, C₁₋₆ alkoxy, pyridinyl, phenylalkyl, phenyl, morpholinyl, pyrrolidinyl, piperidiny, are each optionally substituted with one or more R^{12a}.

In some embodiments, each R^{12a} is independently selected from the group consisting of —F, C₁₋₆ alkyl, C₁₋₆ alkoxy, C₃₋₇ cycloalkyl, and aryl.

In some embodiments, each NR′R″ is separately selected wherein R′ and R″ are each independently selected from the group consisting of —H (hydrogen), —F, —C(O)NR′R″, C₁₋₆ alkyl, C₂₋₆ alkenyl, C₁₋₆ alkoxy, phenyl, phenylalkyl and heteroaryl; or R′ and R″ are taken together with the nitrogen to which they are attached to form heterocyclyl.

In some embodiments, each R^{2c} is independently aryl optionally substituted with halo, cyano, C₁₋₆ alkyl optionally substituted with up to 5 fluoro, or C₁₋₆ alkoxy optionally substituted with up to 5 fluoro, C(O)NR′R″, wherein R′ and R″ are independently optionally substituted C₁₋₆ alkyl. In other embodiments, each R^{2c} is independently heteroaryl or polycyclic moiety, each optionally substituted with aryl, arylalkyl, C₁₋₆ alkyl optionally substituted with up to 5 fluoro, heteroaryl, heterocyclyl, C₃₋₇ cycloalkyl, or C₃₋₇ cycloalkyl-alkyl; wherein said aryl, heteroaryl, and heterocyclyl may be further substituted with C₁₋₆ alkyl, C₁₋₆ alkoxy, halo, or phenyl.

In some embodiments, R¹ is selected from the group consisting of —C(O)OR^{1e}, or optionally substituted heteroaryl and optionally substituted aryl, and R^{1a} is —NHS(O)₂R^{3a} or —NHS(O)₂NR^{3b}R^{3c}; where R^{3a} is selected from the group consisting of C₁₋₆ alkyl and —(CH₂)_{q}C₃₋₇cycloalkyl, each optionally substituted with C₁₋₆ alkyl.

Some embodiments provide compounds of Formula I or Formula Ia, in which R³ is —NHS(O)₂R^{3a} or —NHS(O)₂OR^{3a}, wherein R^{3a} is C₃₋₇₇ cycloalkyl optionally substituted with C₁₋₆ alkyl.

Some embodiments provide compounds of Formula I or Formula Ia, in which R¹ is aryl substituted with one or more substitutents each independently selected from the group consisting of halo, amino, C₁₋₆ alkoxy optionally substituted with up to 5 fluoro, —COOH, —C(O)NR^{1a}R^{1b}, —NHC(O)NR^{1a}R^{1b} and heteroaryl containing 1-3 heteroatoms independently selected from N or O; R² is

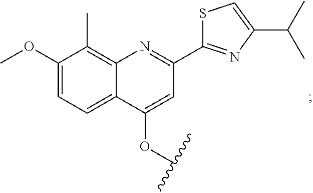

and R³ is —OH, —NHS(O)₂R^{3a}, —NHS(O)₂OR^{3a} or —NHS(O)₂NR^{3b}R^{3c}; where R^{3a} is selected from the group consisting of C₁₋₆ alkyl and —(CH₂)_{q}C₃₋₇cycloalkyl, each optionally substituted with C₁₋₆ alkyl.

In some embodiments, R¹ is aryl substituted with one or more substitutents each independently selected from the group consisting of —C(O)NR^{1a}R^{1b} and —NHC(O)NR^{1a}R^{1b}; R^{1a} and R^{1b} are taken together with the nitrogen to which they are attached to form piperazinyl or morpholinyl, each optionally substituted with one or more substituents independently selected from C₁₋₆ alkyl, C₂₋₆ alkenyl, C₂₋₆ alkynyl, —C(O)OR^{1c}, —C(O)R^{1d}, hydroxy-C₁₋₆alkyl, amino-C₁₋₆alkyl, aryl-C₁₋₆alkyl, aryl optionally substituted with C₁₋₆ alkyl or C₁₋₆ alkyl substituted with up to 5 fluoro, and heteroaryl, wherein in some embodiments, said heteroaryl may contain 1-3 heteroatoms independently selected from N or O; and R^{1c} and R^{1d} are each separately selected from the group consisting of —H, C₁₋₄ alkoxy, C₁₋₆ alkyl, C₃₋₇ cycloalkyl, aryl, arylalkyl, and heteroaryl.

In some embodiments, R¹ is phenyl substituted with one or more substitutents each independently selected from the group consisting of —C(O)NR^{1a}R^{1b}, —NHC(O)NR^{1a}R^{1b} and heteroaryl, wherein in some embodiments, said heteroaryl may contain 1-3 heteroatoms independently selected from N or O; and R³ is —NHS(O)₂R^{3a} or —NHS(O)₂NR^{3b}R^{3c}, where R^{3a} is C₃₋₇cycloalkyl optionally substituted with methyl, and R^{3b} and R^{3c} are methyl.

### Formula II

Some embodiments provide compounds of Formula II:

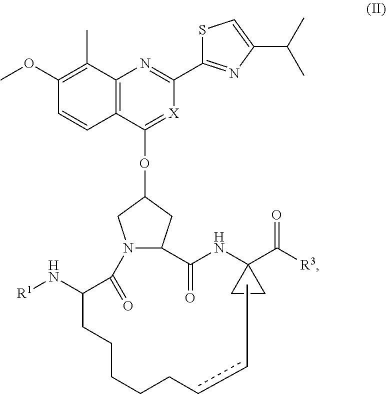

or a pharmaceutically acceptable salt or prodrug thereof wherein X and is —CH— or —N—; R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NHC(O)NR^{1a}R^{1b}, —C(O)OR^{1c}, and heteroaryl. In some embodiments, said heteroaryl may contain 1-3 heteroatoms independently selected from N or O.

R^{1e} is selected from the group consisting of t-butyl, cycloalkyl, and heterocyclyl.

R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl, wherein in some embodiments, said heteroaryl may contain 1-3 heteroatoms independently selected from N or O; R^{1c} and R^{1d} are each separately selected from the group consisting of —H, C₁₋₄ alkoxy, C₁₋₆ alkyl, C₃₋₇ cycloalkyl, aryl, arylalkyl and heteroaryl.

R³ is —OH, —NHS(O)₂R^{3a}, —NHS(O)₂OR^{3a} or —NHS(O)₂NR^{3b}R^{3c}; R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro; and R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{3b} and R^{3c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl.

Each t is independently 0, 1 or 2; and each q is independently 0, 1 or 2.

Any bond represented by a dashed and solid line represents a bond selected from the group

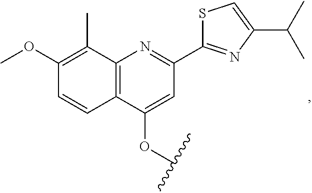

consisting of a single bond and a double bond. Provided that if R² is then R¹ is not —C(O)O-t-butyl, benzoxazyl, t-butylthiazyl, phenyl or phenyl substituted with one or more substituents selected from the group consisting of fluoro, chloro, methyl, —CF₃ and —OCF₃.

In some embodiments, R¹ is selected from the group consisting of —C(O)O-t-butyl and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NHC(O)NR^{1a}R^{1b}, —C(O)OR^{1c}, and heteroaryl. In some embodiments, said heteroaryl may contain 1-3 heteroatoms independently selected from N or O.

In some embodiments, the compound of Formula II is selected from the group consisting of Compounds 901, 101-129, 601-602, 1001-1002, and 1733 as shown in the Examples below.

Some embodiments provide compounds of Formula IIa-1:

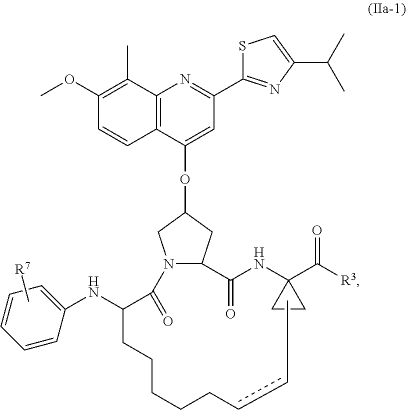

or a pharmaceutically acceptable salt or prodrug thereof wherein R³ is —OH, —NHS(O)₂R^{3a}, —NHS(O)₂OR^{3a} or —NHS(O)₂NR^{3b}R^{3c}; where R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro.

R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{3b} and R^{3c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl.

Each t is independently 0, 1 or 2; and each q is independently 0, 1 or 2.

R⁷ is selected from the group consisting of —NH₂, —NH₂.HCl, —COOH, —C(O)NR^{1a}R^{1b}, —NHC(O)NR^{1a}R^{1b} and heteroaryl containing 1-3 heteroatoms independently selected from N or O; R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl, wherein in some embodiments, said heteroaryl may contain 1-3 heteroatoms independently selected from N or O; R^{1c} and R^{1d} are each separately selected from the group consisting of —H, C₁₋₄ alkoxy, C₁₋₆ alkyl, C₃₋₇ cycloalkyl, aryl, arylalkyl and heteroaryl. Any bond represented by a dashed and solid line represents a bond selected from the group consisting of a single bond and a double bond.

In some embodiments, R³ is —OH, —NHS(O)₂R^{3a}, —NHS(O)₂OR^{3a} or —NHS(O)₂NR^{3b}R^{3c}, where R^{3a} is C₃₋₇cycloalkyl optionally substituted with methyl, and R^{3b} and R^{3c} are methyl; and R⁷ is selected from the group consisting of —NH₂, —NH₂.HCl, —COOH, —C(O)NR^{1a}R^{1b}, —NCH C(O)NR^{1a}R^{1b} and heteroaryl. In some embodiments, said heteroaryl may contain 1-3 heteroatoms independently selected from N or O, wherein R^{1a} and R^{1b} are taken together with the nitrogen to which they are attached to form piperazinyl or morpholinyl, each optionally substituted with one or more substituents independently selected from C₁₋₆ alkyl, —C(O)OR^{1c},

—C(O)R^{1d}, hydroxy-C₁₋₆alkyl, amino-C₁₋₆alkyl, aryl-C₁₋₆alkyl, phenyl optionally substituted with C₁₋₆ alkyl or —CF₃, and heteroaryl, wherein in some embodiments, said heteroaryl may contain 1-3 heteroatoms independently selected from N or O.

### Formula III

Some embodiments provide a compound having the structure of Formula III:

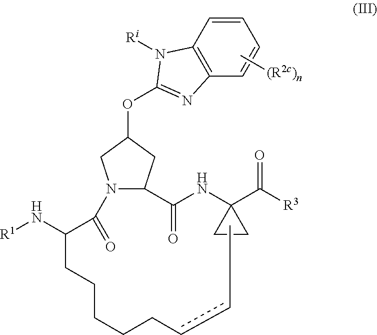

or a pharmaceutically acceptable salt or prodrug thereof, wherein R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NHC(O)NR^{1a}R^{1b}, —C(O)OR^{1c}, and heteroaryl. In some embodiments, said heteroaryl may contain 1-3 heteroatoms independently selected from N or O.

R^{1e} is selected from the group consisting of t-butyl, cycloalkyl, and heterocyclyl; R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl, wherein in some embodiments, said heteroaryl may contain 1-3 heteroatoms independently selected from N or O; and R^{1c} and R^{1d} are each separately selected from the group consisting of —H, C₁₋₄ alkoxy, C₁₋₆ alkyl, C₃₋₇ cycloalkyl, aryl, arylalkyl and heteroaryl.

Rⁱ is C₁₋₆ alkyl optionally substituted with up to 5 fluoro.

Each R^{2c} is independently selected from the group consisting of halo, —C(O)OR^{1c}, —C(O)NR′R″, —NR′R″, —NHC(O)NR′R″, —NHC(O)OR^{1c}, —NHS(O)₂R^{1c}, C₂₋₆ alkyl, C₂₋₆ alkenyl, C₃₋₇ cycloalkyl, C₁₋₆ alkoxy, arylalkyl, polycyclic moiety, aryl, and heteroaryl, said C₂₋₆ alkyl, C₂₋₆ alkenyl, C₃₋₇ cycloalkyl, C₁₋₆ alkoxy, arylalkyl, polycyclic moiety, aryl, and heteroaryl, each optionally substituted with one or more R¹². Each R¹² is independently selected from the group consisting of C₁₋₆ alkyl, C₃₋₇ cycloalkyl, C₁₋₆ alkoxy, heteroaryl, arylalkyl, aryl, —F (fluoro), —Cl (Chloro), —CN, —CF₃, —OCF₃, —C(O)NR′R″, and —NR′R″, wherein said C₂₋₆ alkyl, C₃₋₇ cycloalkyl, C₁₋₆ alkoxy, heteroaryl, arylalkyl, cycloalkylalkyl, and aryl are each optionally substituted with one or more R^{12a}. Each R^{12a} is independently selected from the group consisting of —F, —Cl, —CF₃, —OCF₃, C₁₋₆ alkyl, C₁₋₆ alkoxy, C₃₋₇ cycloalkyl, and aryl.

Each NR′R″ is separately selected wherein R′ and R″ are each independently selected from the group consisting of —H (hydrogen), halo, —C(O)NR′R″, optionally substituted C₁₋₆ alkyl, optionally substituted C₂₋₆ alkenyl, optionally substituted C₁₋₆ alkoxy, optionally substituted aryl, optionally substituted arylalkyl and optionally substituted heteroaryl; or R′ and R″ are taken together with the nitrogen to which they are attached to form heterocyclyl.

R³ is —OH, —NHS(O)₂R^{3a}, —NHS(O)₂OR^{3a} or —NHS(O)₂NR^{3b}R^{3c}; where R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro; wherein R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{3b} and R^{3c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl.

Each t is independently 0, 1 or 2; and each q is independently 0, 1 or 2. n is 1, 2 or 3. Any bond represented by a dashed and solid line represents a bond selected from the group consisting of a single bond and a double bond. Provided that if R² is

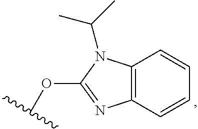

then R¹ is not —C(O)O-t-butyl, phenyl or phenyl substituted with one or more substituents selected from the group consisting of fluoro, chloro and —CF₃; and provided that if R² is

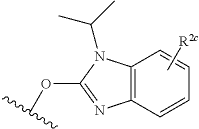

and R^{2c} is —F or methyl, then R¹ is not —C(O)O-t-butyl or phenyl.

In some embodiments, the compound of Formula III is selected from the group consisting of Compounds 201-204, 210-293, 1201-1222, 1401-1436, 1701-1732, and 1734-1778 as shown in the Examples below.

In some embodiments, each R^{2c} is independently selected from the group consisting of —CF₃, —Br(bromo), —Cl(chloro), —C(O)OH, —C(O)NR′R″, —NR′R″, —NHC(O)NR′R″, —NHC(O)OR^{1c}, —NHS(O)₂R^{1c}, C₂₋₆ alkyl, C₂₋₆ alkenyl, C₁₋₆ alkoxy, polycyclic moiety, phenyl, and heteroaryl, said C₂₋₆ alkyl, C₂₋₆ alkenyl, C₁₋₆ alkoxy, polycyclic moiety, aryl, and heteroaryl, each optionally substituted with one or more R¹²; and in some embodiments, said heteroaryl may be selected from the group consisting of furanyl, thiazolyl, oxazolyl, thiophenyl, pyrazolyl, and benzothiazolyl.

Each R¹² is independently selected from the group consisting of C₁₋₆ alkyl, C₃₋₇ cycloalkyl, C₁₋₆ alkoxy, pyridinyl, phenylalkyl, phenyl, —F (fluoro), —Cl (Chloro), —CN, —CF₃, —OCF₃, —C(O)NR′R″, —NR′R″, C₃₋₇ cycloalkyl-alkyl, wherein said C₁₋₆ alkyl, C₃₋₇ cycloalkyl, C₁₋₆ alkoxy, pyridinyl, phenylalkyl, phenyl, and —NR′R″ are each optionally substituted with one or more R^{12a}.

In some embodiments, each R^{12a} is independently selected from the group consisting of —F, —C₁, C₁₋₆ alkyl, C₁₋₆ alkoxy, C₃₋₇ cycloalkyl, and aryl.

In some embodiments, each NR′R″ is separately selected wherein R′ and R″ are each independently selected from the group consisting of —H (hydrogen), —F, —Cl, —C(O)NR′R″, C₁₋₆ alkyl, C₂₋₆ alkenyl, C₁₋₆ alkoxy, phenyl, phenylalkyl and heteroaryl; or R′ and R″ are taken together with the nitrogen to which they are attached to form heterocyclyl. In some embodiments, said heterocyclyl may be morpholinyl, pyrrolidinyl, or piperidinyl.

In some embodiments, each R^{2c} is independently aryl optionally substituted with halo, cyano, C₁₋₆ alkyl optionally substituted with up to 5 fluoro, or C₁₋₆ alkoxy optionally substituted with up to 5 fluoro, C(O)NR′R″, wherein R′ and R″ are independently optionally substituted C₁₋₆ alkyl. In other embodiments, each R^{2c} is independently heteroaryl or polycyclic moiety, each optionally substituted with aryl, arylalkyl, C₁₋₆ alkyl optionally substituted with up to 5 fluoro, heteroaryl, heterocyclyl, C₃₋₇ cycloalkyl, or C₃₋₇ cycloalkyl-alkyl; wherein said aryl, heteroaryl, and heterocyclyl may be further substituted with C₁₋₆ alkyl, C₁₋₆ alkoxy, halo, or phenyl.

In some embodiments, R¹ is selected from the group consisting of —C(O)O-t-butyl and phenyl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NHC(O)NR^{1a}R^{1b}, —C(O)OR^{1c}, and heteroaryl; in some embodiments, said heteroaryl may contain 1-3 heteroatoms independently selected from N or O; and R³ is —OH, —NHS(O)₂R^{3a} or —NHS(O)₂NR^{3b}R^{3c}, where R^{3a} is C₃₋₇cycloalkyl optionally substituted with C₁₋₆ alkyl, and R^{3b} and R^{3c} are independently selected from —H or C₁₋₆ alkyl.

In some embodiments, R¹ is selected from the group consisting of —C(O)O-t-butyl and phenyl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, and C₂₋₆ alkynyl; and R³ is —OH, —NHS(O)₂R^{3a} or —NHS(O)₂NR^{3b}R^{3c}, where R^{3a} is C₃₋₇cycloalkyl optionally substituted with C₁₋₆ alkyl, and R^{3b} and R^{3c} are independently selected from —H or C₁₋₆ alkyl.

Some embodiments provide a compound having the structure of Formula IIIa or nth:

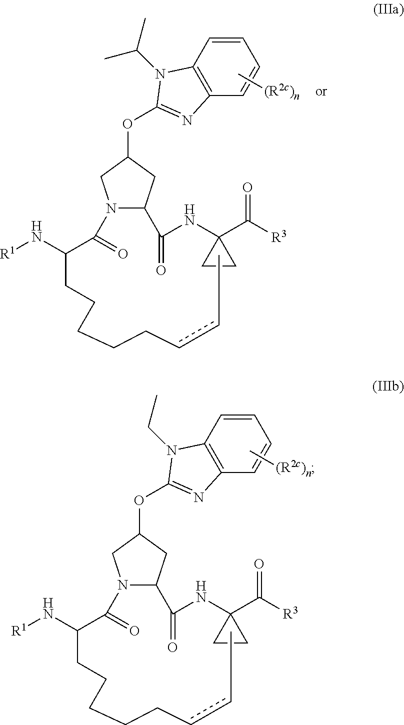

wherein R¹, R^{2c}, R³, K and n are the same as defined above.

In some embodiments, each R^{2c} is independently selected from the group consisting of —CF₃, —Br(bromo), —Cl(chloro), —C(O)OH, —C(O)NR′R″, —NR′R″, —NHC(O)NR′R″, —NHC(O)OR^{1c}, —NHS(O)₂R^{1c}, C₂₋₆ alkyl, C₂₋₆ alkenyl, C₁₋₆ alkoxy, polycyclic moiety, phenyl, and heteroaryl, said C₂₋₆ alkyl, C₂₋₆ alkenyl, C₁₋₆ alkoxy, polycyclic moiety, aryl, and heteroaryl, each optionally substituted with one or more R¹²; and in some embodiments, said heteroaryl may be selected from the group consisting of furanyl, thiazolyl, oxazolyl, thiophenyl, pyrazolyl, and benzothiazolyl.

Each R¹² is independently selected from the group consisting of C₁₋₆ alkyl, C₃₋₇ cycloalkyl, C₁₋₆ alkoxy, pyridinyl, phenylalkyl, phenyl, —F (fluoro), —Cl (Chloro), —CN, —CF₃, —OCF₃, —C(O)NR′R″, —NR′R″, C₃₋₇ cycloalkyl-alkyl, wherein said C₁₋₆ alkyl, C₃₋₇ cycloalkyl, C₁₋₆ alkoxy, pyridinyl, phenylalkyl, phenyl, and —NR′R″ are each optionally substituted with one or more R^{12a}.

In some embodiments, each R^{12a} is independently selected from the group consisting of —F, —C₁, C₁₋₆ alkyl, C₁₋₆ alkoxy, C₃₋₇ cycloalkyl, and aryl.

In some embodiments, each NR′R″ is separately selected wherein R′ and R″ are each independently selected from the group consisting of —H (hydrogen), —F, —Cl, —C(O)NR′R″, C₁₋₆ alkyl, C₂₋₆ alkenyl, C₁₋₆ alkoxy, phenyl, phenylalkyl and heteroaryl; or R′ and R″ are taken together with the nitrogen to which they are attached to form heterocyclyl. In some embodiments, said heterocyclyl may be morpholinyl, pyrrolidinyl, or piperidinyl.

In some embodiments, each R^{2c} is independently aryl optionally substituted with one or more substituents selected from the group consisting of halo, cyano, C₁₋₆ alkyl optionally substituted with up to 5 fluoro, or C₁₋₆ alkoxy optionally substituted with up to 5 fluoro, C(O)NR′R″, wherein R′ and R″ are independently optionally substituted C₁₋₆ alkyl. In other embodiments, each R^{2c} is independently heteroaryl or polycyclic moiety, each optionally substituted with —CF₃, aryl, arylalkyl, C₁₋₆ alkyl optionally substituted with up to 5 fluoro, heteroaryl, heterocyclyl, C₃₋₇ cycloalkyl, or C₃₋₇ cycloalkyl-alkyl; wherein said aryl, heteroaryl, and heterocyclyl may be further substituted with C₁₋₆ alkyl, C₁₋₆ alkoxy, halo, or phenyl.

In some embodiments, the compound may have the structure of formula (IIIa-1):

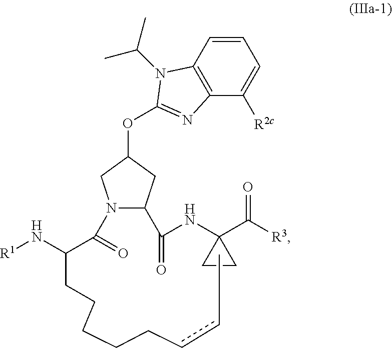

wherein R¹, R^{2c} and R³ are as defined for Formula IIIa or IIIb.

In some embodiments, R^{2c} in Formula IIIa or IIIb is selected from the group consisting of —CF₃, —Br(bromo), —Cl(chloro), —C(O)OH, —C(O)NR′R″, —NR′R″, —NHC(O)NR′R″, —NHC(O)OR^{1c}, —NHS(O)₂R^{1c}, C₂₋₆ alkyl, C₂₋₆ alkenyl, C₁₋₆ alkoxy, polycyclic moiety, phenyl, and heteroaryl, said C₂₋₆ alkyl, C₂₋₆ alkenyl, C₁₋₆ alkoxy, polycyclic moiety, aryl, and heteroaryl, each optionally substituted with one or more R¹²; and in some embodiments, said heteroaryl may be selected from the group consisting of furanyl, thiazolyl, oxazolyl, thiophenyl, pyrazolyl, and benzothiazolyl.

Each R¹² is independently selected from the group consisting of C₁₋₆ alkyl, C₃₋₇ cycloalkyl, C₁₋₆ alkoxy, pyridinyl, phenylalkyl, phenyl, —F (fluoro), —Cl(Chloro), —CN, —CF₃, —OCF₃, —C(O)NR′R″, —NR′R″, C₃₋₇ cycloalkyl-alkyl, wherein said C₁₋₆ alkyl, C₃₋₇ cycloalkyl, C₁₋₆ alkoxy, pyridinyl, phenylalkyl, phenyl, and —NR′R″ are each optionally substituted with one or more R^{12a}.

In some embodiments, each R^{12a} is independently selected from the group consisting of —F, C₁₋₆ alkyl, C₁₋₆ alkoxy, C₃₋₇ cycloalkyl, and aryl.

In some embodiments, each NR′R″ is separately selected wherein R′ and R″ are each independently selected from the group consisting of —H (hydrogen), —F, —C(O)NR′R″, C₁₋₆ alkyl, C₂₋₆ alkenyl, C₁₋₆ alkoxy, phenyl, phenylalkyl and heteroaryl; or R′ and R″ are taken together with the nitrogen to which they are attached to form heterocyclyl. In some embodiments, said heterocyclyl may be morpholinyl, pyrrolidinyl, or piperidinyl.

In some embodiments, R^{1c} is selected from the group consisting of C₁₋₆ alkyl, aryl and arylalkyl.

### Formula IV

Some embodiments provide a compound having the structure of Formula IV:

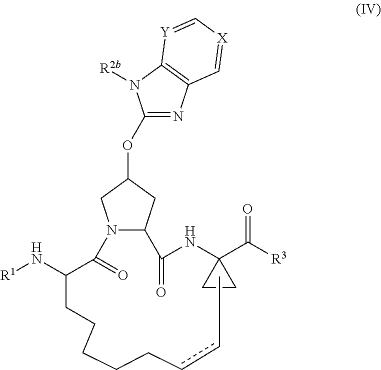

or a pharmaceutically acceptable salt or prodrug thereof wherein R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NCH(O)NR^{1a}R^{1b}, —C(O)OR^{1c}, and heteroaryl; in some embodiments, said heteroaryl may contain 1-3 heteroatoms independently selected from N or O.

R^{1e} is selected from the group consisting of t-butyl, cycloalkyl, and heterocyclyl; R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl, wherein in some embodiments, said heteroaryl may contain 1-3 heteroatoms independently selected from N or O; R^{1c} and R^{1d} are each separately selected from the group consisting of —H, C₁₋₄ alkoxy, C₁₋₆ alkyl, C₃₋₇ cycloalkyl, aryl, arylalkyl and heteroaryl.

X and Y are each independently selected from —CH— or —N—, wherein X and Y are not both —CH—.

R³ is —OH, —NHS(O)₂R^{3a}, —NHS(O)₂OR^{3a} or —NHS(O)₂NR^{3b}R^{3c}; where R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro; wherein R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{3b} and R^{3c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl.

Each t is independently 0, 1 or 2; and each q is independently 0, 1 or 2. Any bond represented by a dashed and solid line represents a bond selected from the group consisting of a single bond and a double bond.

Some embodiments provide a compound having the structure selected from the group consisting of Compounds 209 and 501-504.

Some embodiments provide a compound having the structure of Formula IVa or IVb:

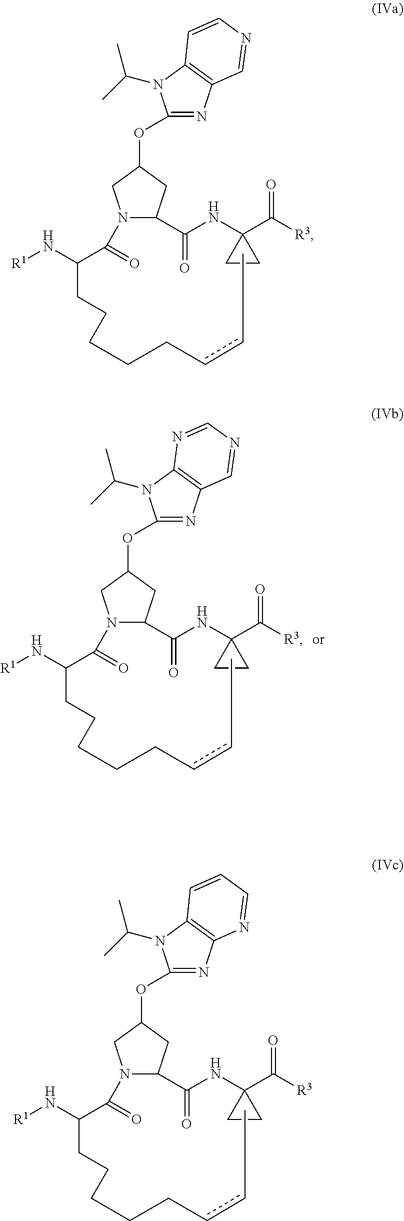

wherein R¹ and R³ are as defined above.

In some embodiments, in any one of Formulas IV, IVa. IVb and IVc, R¹ is selected from the group consisting of —C(O)OR^{1e}, optionally substituted heteroaryl, and phenyl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NHC(O)NR^{1a}R^{1b}, —C(O)OR^{1c}, and heteroaryl containing 1-3 heteroatoms independently selected from N or O; and R³ is —OH, —NHS(O)₂R^{3a} or —NHS(O)₂NR^{3b}R^{3c}, where R^{3a} is C₃₋₇cycloalkyl optionally substituted with methyl, and R^{3b} and R^{3c} are methyl.

Some embodiments provide a compound having the structure of Formula III or IV

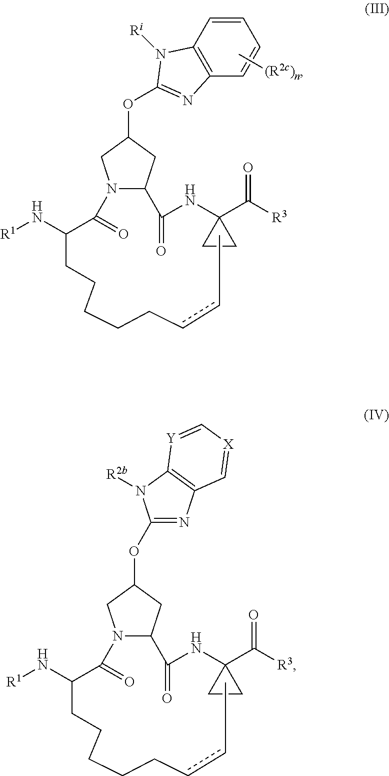

or a pharmaceutically acceptable salt or prodrug thereof wherein: R¹ is selected from the group consisting of —C(O)OR^{1e}, optionally substituted heteroaryl containing 1-3 heteroatoms independently selected from S, N or O, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NHC(O)NR^{1a}R^{1b}, —C(O)OR^{1c}, and heteroaryl containing 1-3 heteroatoms independently selected from N or O.

R^{1e} is selected from the group consisting of t-butyl, cycloalkyl, and heterocyclyl containing 1-3 heteroatoms independently selected from N, O and S. R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl containing 1-3 heteroatoms independently selected from N and O. R^{1c} and R^{1d} are each separately selected from the group consisting of —H, C₁₋₄ alkoxy, linear and branched C₁₋₆ alkyl, C₃₋₇ cycloalkyl, aryl, arylalkyl and heteroaryl containing 1-3 heteroatoms independently selected from N, O and S.

X and Y are each independently selected from —CH— or —N—, wherein X and Y are not both —CH—; (c) R^{2b} is selected from the group consisting of linear and branched C₁₋₆ alkyl optionally substituted with up to 5 fluoro, C₂₋₆ alkenyl, C₃₋₇ cycloalkyl, arylalkyl, optionally substituted aryl and optionally substituted heteroaryl containing 1-3 heteroatoms independently selected from S, N or O.

Each R^{2c} is independently selected from the group consisting of —Br, —Cl, —CF₃, C₂₋₆ alkyl, C₂₋₆ alkenyl, —C(O)NR′R″, —NR′R″, —NHC(O)NR′R″, —NHC(O)OR^{1c}, —NHS(O)₂R^{1c}, —C(O)OH, aryl and heteroaryl containing 1-3 heteroatoms independently selected from S, N or O, wherein the heteroaryl is optionally substituted with one or more substituents selected from the group consisting of —CF₃, linear and branched C₁₋₆ alkyl, C₃₋₇ cycloalkyl, arylalkyl and aryl, and the aryl is optionally substituted with one or more substituents selected from the group consisting of —F, —CN, —CF₃, —OCF₃, C₁₋₆ alkyl, C₁₋₆ alkoxy, and C(O)NR′R″; wherein R′ and R″ are each independently selected from the group consisting of —H, optionally substituted C₁₋₆ alkyl, optionally substituted C₂₋₆ alkenyl, optionally substituted aryl, optionally substituted arylalkyl and optionally substituted heteroaryl containing 1-3 heteroatoms independently selected from S, N or O.

Rⁱ is C₁₋₆ alkyl optionally substituted with up to 5 fluoro. R³ is —OH, —NHS(O)₂R^{3a}, —NHS(O)₂OR^{3a} or —NHS(O)₂NR^{3b}R^{3c}; where R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro.

Wherein R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{3b} and R^{3c} are taken together with the nitrogen to which they are attached to form a three- to six-membered heterocyclic ring containing 1-3 heteroatoms independently selected from S, N or O, and the heterocylic ring is optionally substituted with one or more substituents each independently selected from the group consisting of halo, cyano, nitro, C₁₋₆ alkyl, C₁₋₆ alkoxy, and phenyl.

Each t is independently 0, 1 or 2; and each q is independently 0, 1 or 2. n is 1, 2 or 3; and any bond represented by a dashed and solid line represents a bond selected from the group consisting of a single bond and a double bond Formula V

Some embodiments provide a compound having the structure of Formula V:

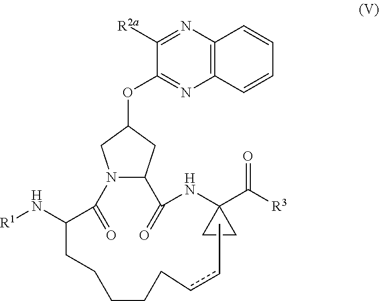

or a pharmaceutically acceptable salt or prodrug thereof wherein R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NCH(O)NR^{1a}R^{1b}, —C(O)OR^{1c}, and heteroaryl; in some embodiments, said heteroaryl may contain 1-3 heteroatoms independently selected from N or O.

R^{1e} is selected from the group consisting of t-butyl, cycloalkyl, and heterocyclyl; R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl, wherein in some embodiments, said heteroaryl may contain 1-3 heteroatoms independently selected from N or O; R^{1c} and R^{1d} are each separately selected from the group consisting of —H, C₁₋₄ alkoxy, C₁₋₆ alkyl, C₃₋₇ cycloalkyl, aryl, arylalkyl and heteroaryl.

R^{2a} is selected from the group consisting of —H, —C(O)OR^{1c}, C₁₋₆ alkyl optionally substituted with up to 5 fluoro, C₂₋₆ alkenyl, C₃₋₇ cycloalkyl, optionally substituted aryl and optionally substituted heteroaryl.

R³ is —OH, —NHS(O)₂R^{3a}, —NHS(O)₂OR^{3a} or —NHS(O)₂NR^{3b}R^{3c}; where R^{1a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro; wherein R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{3b} and R^{1c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl.

Each t is independently 0, 1 or 2; and each q is independently 0, 1 or 2. Provided that if R² is

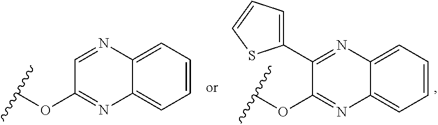

then R¹ is not phenyl.

Any bond represented by a dashed and solid line represents a bond selected from the group consisting of a single bond and a double bond.

In some embodiments, R¹ is selected from the group consisting of —C(O)O-t-butyl and phenyl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, and C₂₋₆ alkynyl; and R³ is —OH, —NHS(O)₂R^{3a} or —NHS(O)₂NR^{3b}R^{3c}, where R^{3a} is C₃₋₇cycloalkyl optionally substituted with C₁₋₆ alkyl, and R^{3b} and R^{1c} are independently selected from —H or C₁₋₆ alkyl. Some embodiments provide a compound of Formula V selected from the group consisting of Compounds 301-312.

### Formula VI

Some embodiments provide a compound having the structure of Formula VI-1 or VI-2:

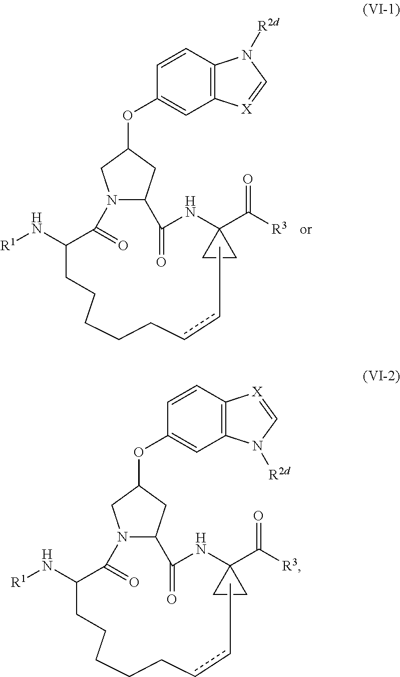

or a pharmaceutically acceptable salt or prodrug thereof, wherein X is —N— or —CH—, R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NHC(O)NR^{1a}R^{1b}, —C(O)OR^{1c}, and heteroaryl; in some embodiments, said heteroaryl may contain 1-3 heteroatoms independently selected from N or O.

R^{1e} is selected from the group consisting of t-butyl, cycloalkyl, and heterocyclyl; R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl, wherein in some embodiments, said heteroaryl may contain 1-3 heteroatoms independently selected from N or O; R^{1c} and R^{1d} are each separately selected from the group consisting of —H, C₁₋₄ alkoxy, C₁₋₆ alkyl, C₃₋₇ cycloalkyl, aryl, arylalkyl and heteroaryl.

R^{2d} is selected from the group consisting of C₁₋₆ alkyl optionally substituted with up to 5 fluoro, C₂₋₆ alkenyl, C₃₋₇ cycloalkyl, arylalkyl, optionally substituted aryl and optionally substituted heteroaryl.

R³ is —OH, —NHS(O)₂R^{3a}, —NHS(O)₂OR^{3a} or —NHS(O)₂NR^{3b}R^{3c}; where R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro; wherein R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{3b} and R^{3c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl.

Each t is independently 0, 1 or 2; and each q is independently 0, 1 or 2. Any bond represented by a dashed and solid line represents a bond selected from the group consisting of a single bond and a double bond.

In some embodiments, the compound may have the structure of one of the following formulas:

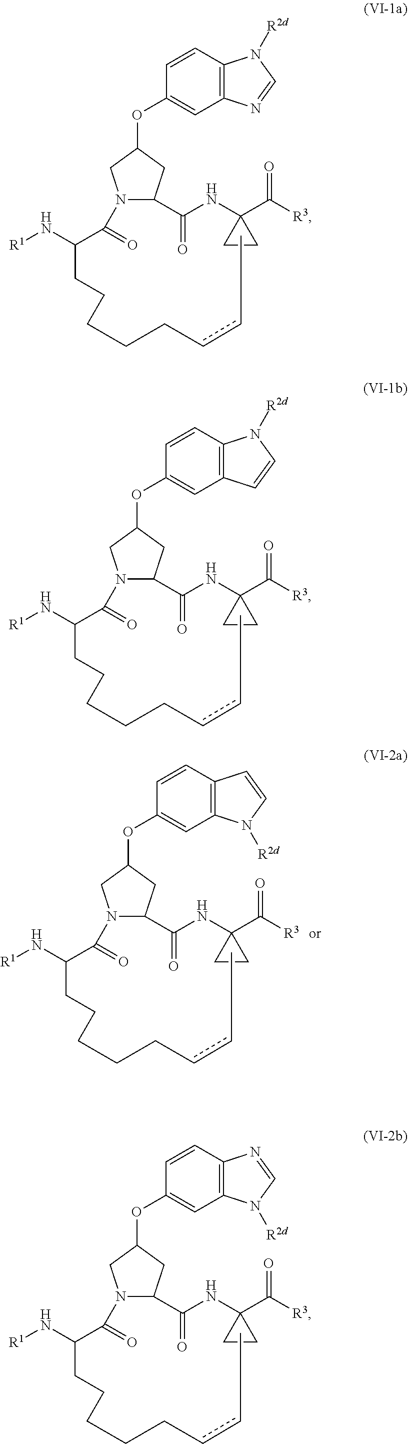

wherein R¹, R³ and R^{2d} are as defined above.

In some embodiments, R¹ may be selected from the group consisting of —C(O)O-t-butyl, and R³ is —OH, —NHS(O)₂R^{3a} or —NHS(O)₂NR^{3b}R^{3c}, where R^{3a} is C₃₋₇ cycloalkyl optionally substituted with methyl, and R^{3b} and R^{3c} are methyl.

In some embodiments, R^{2d} is selected from the group consisting of C₁₋₆ alkyl optionally substituted with up to 5 fluoro and optionally substituted aryl. In some embodiments, R^{2d} is methyl, ethyl, i-propyl or phenyl.

Some embodiments provide a compound of Formula VI selected from the group consisting of Compounds 294-299 and 701-702.

In some embodiments, R¹ is selected from the group consisting of —C(O)O-t-butyl and phenyl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, and C₂₋₆ alkynyl; and R³ is —OH, —NHS(O)₂R^{3a} or —NHS(O)₂NR^{3b}R^{3c}, where R^{3a} is C₃₋₇cycloalkyl optionally substituted with C₁₋₆ alkyl, and R^{3b} and R^{3c} are independently selected from —H or C₁₋₆ alkyl.

### Formula VII

Some embodiments provide a compound having the structure of Formula VII:

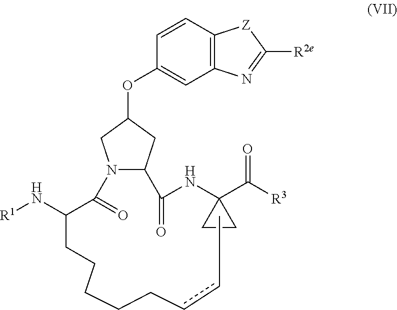

or a pharmaceutically acceptable salt or prodrug thereof, wherein Z is O or S; R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NHC(O)NR^{1a}R^{1b}, —C(O)OR^{1c}, and heteroaryl; in some embodiments, said heteroaryl may contain 1-3 heteroatoms independently selected from N or O.

R^{1e} is selected from the group consisting of t-butyl, cycloalkyl, and heterocyclyl; R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl, wherein in some embodiments, said heteroaryl may contain 1-3 heteroatoms independently selected from N or O; R^{1c} and R^{1d} are each separately selected from the group consisting of —H, C₁₋₄ alkoxy, C₁₋₆ alkyl, C₃₋₇ cycloalkyl, aryl, arylalkyl and heteroaryl.

R^{2e} is selected from the group consisting of —H, halo, —C(O)OR^{1c}, —C(O)NR′R″, —NR′R″, —NHC(O)NR′R″, C₁₋₆ alkyl optionally substituted with up to 5 fluoro, C₂₋₆ alkenyl, C₃₋₇ cycloalkyl, optionally substituted C₁₋₆ alkoxy, optionally substituted aryl and optionally substituted heteroaryl; wherein R′ and R″ are each independently selected from the group consisting of —H, optionally substituted C₁₋₆ alkyl, optionally substituted C₂₋₆ alkenyl, optionally substituted aryl, optionally substituted arylalkyl and optionally substituted heteroaryl.

R³ is —OH, —NHS(O)₂R^{3a}, —NHS(O)₂OR^{3a} or —NHS(O)₂NR^{3b}R^{3c}; where R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro; wherein R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{3b} and R^{3c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl

Each t is independently 0, 1 or 2; each q is independently 0, 1 or 2; and any bond represented by a dashed and solid line represents a bond selected from the group consisting of a single bond and a double bond.

In some embodiments, the compound may have the structure of one of the following formulas:

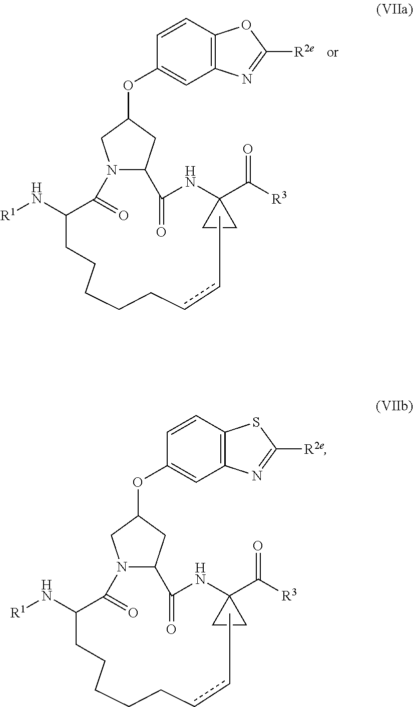

wherein R¹, R³ and R^{2e} are as defined above.

Some embodiments provide a compound of Formula VII selected from the group consisting of Compounds 1251-1253.

In some embodiments, in Formula VII, VIIa or VIIb, R¹ is selected from the group consisting of —C(O)O-t-butyl and phenyl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, and C₂₋₆ alkynyl; and R³ is —OH, —NHS(O)₂R^{3a} or —NHS(O)₂NR^{3b}R^{3c}, where R^{3a} is C₃₋₇cycloalkyl optionally substituted with C₁₋₆ alkyl, and R^{3b} and R^{3c} are independently selected from —H or C₁₋₆ alkyl.

### Formula VIII

Some embodiments provide a compound having the structure of Formula VIII:

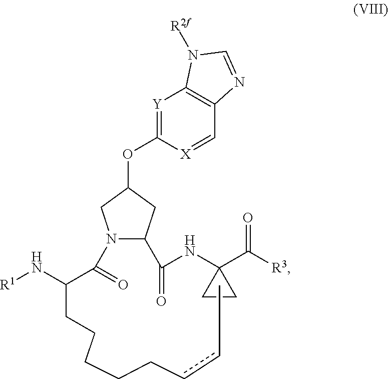

or a pharmaceutically acceptable salt or prodrug thereof, wherein R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NHC(O)NR^{1a}R^{1b}, —C(O)OR^{1c}, and heteroaryl; in some embodiments, said heteroaryl may contain 1-3 heteroatoms independently selected from N or O.

R^{1e} is selected from the group consisting of t-butyl, cycloalkyl, and heterocyclyl.

R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl, wherein in some embodiments, said heteroaryl may contain 1-3 heteroatoms independently selected from N or O; R^{1c} and R^{1d} are each separately selected from the group consisting of —H, C₁₋₄ alkoxy, C₁₋₆ alkyl, C₃₋₇ cycloalkyl, aryl, arylalkyl and heteroaryl.

X and Y are each independently selected from —CH— or —N—, wherein X and Y are not both —CH—; R^{2f} is selected from the group consisting of C₁₋₆ alkyl optionally substituted with up to 5 fluoro, C₂₋₆ alkenyl, C₃₋₇ cycloalkyl, arylalkyl, optionally substituted aryl and optionally substituted heteroaryl;

R³ is —OH, —NHS(O)₂R^{3a}, —NHS(O)₂OR^{3a} or —NHS(O)₂NR^{3b}R^{3c}; where R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro.

Wherein R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{3b} and R^{3c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl.

Each t is independently 0, 1 or 2; each q is independently 0, 1 or 2; any bond represented by a dashed and solid line represents a bond selected from the group consisting of a single bond and a double bond.

Some embodiments provide a compound having the structure of Formula VIIIa:

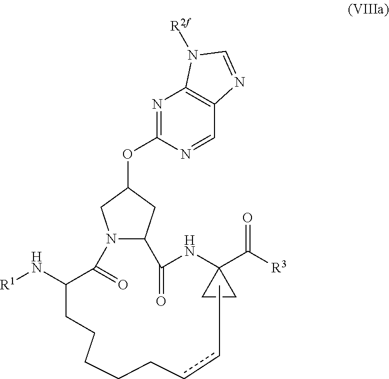

or a pharmaceutically acceptable salt or prodrug thereof wherein R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NCH(O)NR^{1a}R^{1b}, —C(O)OR^{1c}, and heteroaryl; in some embodiments, said heteroaryl may contain 1-3 heteroatoms independently selected from N or O;

R^{1e} is selected from the group consisting of t-butyl, cycloalkyl, and heterocyclyl.

R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl, wherein in some embodiments, said heteroaryl may contain 1-3 heteroatoms independently selected from N or O; R^{1c} and R^{1d} are each separately selected from the group consisting of —H, C₁₋₄ alkoxy, C₁₋₆ alkyl, C₃₋₇ cycloalkyl, aryl, arylalkyl and heteroaryl.

R^{2f} is selected from the group consisting of C₁₋₆ alkyl optionally substituted with up to 5 fluoro, C₂₋₆ alkenyl, C₃₋₇ cycloalkyl, arylalkyl, optionally substituted aryl and optionally substituted heteroaryl.

R³ is —OH, —NHS(O)₂R^{3a}, —NHS(O)₂OR^{3a} or —NHS(O)₂NR^{3b}R^{3c}; where R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro.

Wherein R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{3b} and R^{3c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl.

Each t is independently 0, 1 or 2; each q is independently 0, 1 or 2; and any bond represented by a dashed and solid line represents a bond selected from the group consisting of a single bond and a double bond.

Some embodiments provide a compound of Formula VIII selected from Compound 505 or 506.

In some embodiments, R¹ is selected from the group consisting of —C(O)O-t-butyl and phenyl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, and C₂₋₆ alkynyl; and R³ is —OH, —NHS(O)₂R^{3a} or —NHS(O)₂NR^{3b}R^{3c}, where R^{3a} is C₃₋₇cycloalkyl optionally substituted with C₁₋₆ alkyl, and R^{3b} and R^{3c} are independently selected from —H or C₁₋₆ alkyl.

### Formula IX

Some embodiments provide a compound having the structure of Formula IX:

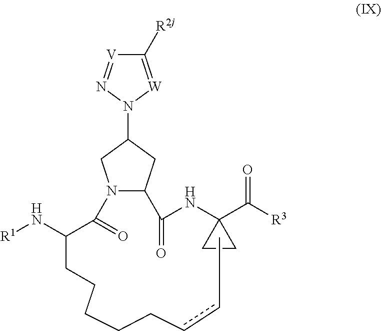

or a pharmaceutically acceptable salt or prodrug thereof, wherein V and W are each independently selected from —CR^{2k}— or —N—, wherein V and W are not both —CR^{2k}—; R^{2j} and R^{2k} are each independently selected from the group consisting of H, halo, optionally substituted aryl, optionally substituted heteroaryl; or R^{2j} and R^{2k} together form an aryl ring optionally substituted by 1-3 R^{2g}.

R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NCH(O)NR^{1a}R^{1b}, —C(O)OR^{1c}, and heteroaryl; in some embodiments, said heteroaryl may contain 1-3 heteroatoms independently selected from N or O; and R^{1e} is selected from the group consisting of t-butyl, cycloalkyl, and heterocyclyl.

R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl, wherein in some embodiments, said heteroaryl may contain 1-3 heteroatoms independently selected from N or O; R^{1c} and R^{1d} are each separately selected from the group consisting of —H, C₁₋₄ alkoxy, C₁₋₆ alkyl, C₃₋₇ cycloalkyl, aryl, arylalkyl and heteroaryl.

R^{2g} is selected from the group consisting of —H, halo, —C(O)OR^{1c}, —C(O)NR′R″, —NR′R″, —NHC(O)NR′R″, C₁₋₆ alkyl optionally substituted with up to 5 fluoro, C₂₋₆ alkenyl, C₃₋₇ cycloalkyl, optionally substituted C₁₋₆ alkoxy, optionally substituted aryl and optionally substituted heteroaryl; R′ and R″ are each independently selected from the group consisting of —H, optionally substituted C₁₋₆ alkyl, optionally substituted C₂₋₆ alkenyl, optionally substituted aryl, optionally substituted arylalkyl and optionally substituted heteroaryl.

R³ is —OH, —NHS(O)₂R^{3a}, —NHS(O)₂OR^{3a} or —NHS(O)₂NR^{3b}R^{3c}; where R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro.

Wherein R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{3b} and R^{3c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl.

Each t is independently 0, 1 or 2; each q is independently 0, 1 or 2; and any bond represented by a dashed and solid line represents a bond selected from the group consisting of a single bond and a double bond.

Some embodiments provide a compound of Formula IX selected from the following formulae:

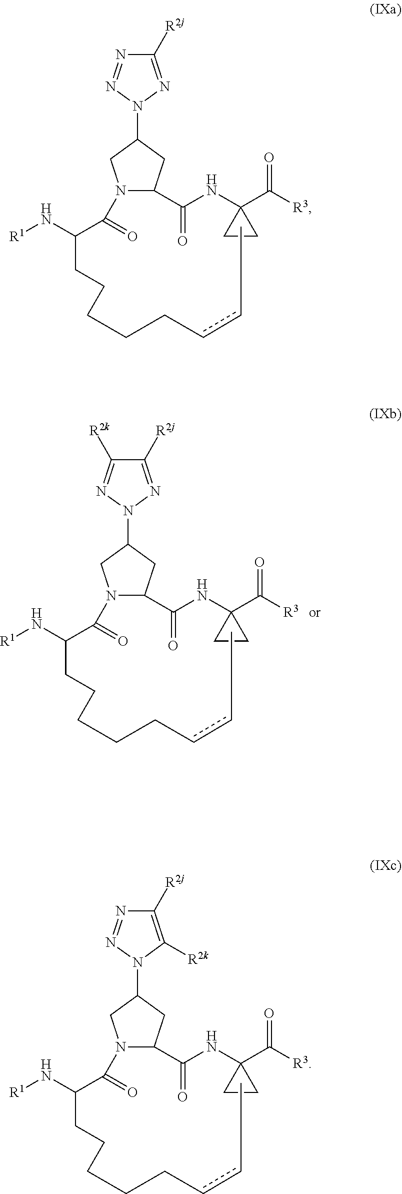

Some embodiments provide a compound of Formula IX selected from the group consisting of Compounds 801-805 and 1501-1506.

In some embodiments, R¹ is selected from the group consisting of —C(O)O-t-butyl and phenyl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, and C₂₋₆ alkynyl; and R³ is —OH, —NHS(O)₂R^{3a} or —NHS(O)₂NR^{3b}R^{3c}, where R^{3a} is C₃₋₇cycloalkyl optionally substituted with C₁₋₆ alkyl, and R^{3b} and R^{3c} are independently selected from —H or C₁₋₆ alkyl.

### Formula X

Some embodiments provide a compound having the structure of Formula X:

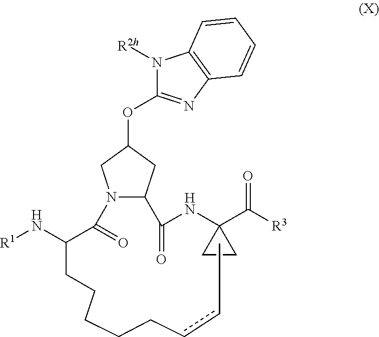

or a pharmaceutically acceptable salt or prodrug thereof wherein R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NCH(O)NR^{1a}R^{1b}, —C(O)OR^{1c}, and heteroaryl; in some embodiments, said heteroaryl may contain 1-3 heteroatoms independently selected from N or O;

R^{1e} is selected from the group consisting of t-butyl, cycloalkyl, and heterocyclyl; R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl, wherein in some embodiments, said heteroaryl may contain 1-3 heteroatoms independently selected from N or O; R^{1c} and R^{1d} are each separately selected from the group consisting of —H, C₁₋₄ alkoxy, C₁₋₆ alkyl, C₃₋₇ cycloalkyl, aryl, arylalkyl and heteroaryl.

R^{2h} is selected from the group consisting of n-propyl, cyclopropyl, n-butyl, t-butyl, 1-sec-butyl and phenyl.

R³ is —OH, —NHS(O)₂R^{3a}, —NHS(O)₂OR^{3a} or —NHS(O)₂NR^{3b}R^{3c}; where R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro.

Wherein R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{3b} and R^{3c} are taken together with the nitrogen to which they are attached to form a three- to six-membered heterocyclic ring, bonded to the parent structure through a nitrogen, and the heterocylic ring is optionally substituted with one or more substituents each independently selected from the group consisting of halo, cyano, nitro, C₁₋₆ alkyl, C₁₋₆ alkoxy, and phenyl.

Each t is independently 0, 1 or 2; each q is independently 0, 1 or 2; and any bond represented by a dashed and solid line represents a bond selected from the group consisting of a single bond and a double bond.

Some embodiments provide a compound of Formula X selected from the group consisting of Compounds 200 and 205-208.

In some embodiments, R¹ is selected from the group consisting of —C(O)O-t-butyl and phenyl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, and C₂₋₆ alkynyl; and R³ is —OH, —NHS(O)₂R^{3a} or —NHS(O)₂NR^{3b}R^{3c}, where R^{3a} is C₃₋₇cycloalkyl optionally substituted with C₁₋₆ alkyl, and R^{3b} and R^{3c} are independently selected from —H or C₁₋₆ alkyl.

### Formula XI

The present embodiments provide compounds having the formula XI:

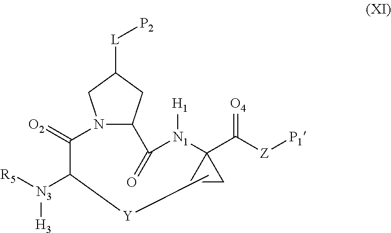

or a pharmaceutically acceptable salt, prodrug, or ester thereof wherein:

(a) Z is a group configured to hydrogen bond to an NS3 protease His57 imidazole moiety, and to hydrogen bond with the hydrogen and nitrogen of the backbone amide group of the NS3 amino acid at position 137;

(b) P₁′ is a group configured to form a non-polar interaction with at least one NS3 protease 51′ pocket moiety selected from the group consisting of Lys136, Gly137, Ser139, His 57, Gly58, Gln41, Ser42, and Phe43;

(g) L is a linker group consisting of from 1 to 5 atoms selected from the group consisting of carbon, oxygen, nitrogen, hydrogen, and sulfur;

(h) P₂ is selected from the group consisting of unsubstituted aryl, substituted aryl, unsubstituted heteroaryl, substituted heteroaryl, unsubstituted heterocyclic and substituted heterocyclic; P₂ being configured to form a non-polar interaction with at least one NS3 protease S2 pocket moiety selected from the group consisting of Tyr56, Gly58, Ala59, Gly60, Gln41, His57, Va178, Asp79, Gln80 and Asp81, and P₂ being configured so that no atom of P2 makes a nonpolar interaction with an epsilon, zeta, or eta sidechain atom of the amino acid at position 155;

(i) R⁵ is selected from the group consisting of H, C(O)NR⁶R⁷ and C(O)OR⁸;

(j) R⁶ and R⁷ are each independently H, C₁₋₆ alkyl, C₃₋₇ cycloalkyl, C₄₋₁₀ alkylcycloalkyl or phenyl, said phenyl optionally substituted by up to three halo, cyano, nitro, hydroxy, C₃₋₇ cycloalkyl, C₄₋₁₀ alkylcycloalkyl, C₂₋₆ alkenyl, hydroxy-C₁₋₆ alkyl, C₁₋₆ alkyl optionally substituted with up to 5 fluoro, C₁₋₆ alkoxy optionally substituted with up to 5 fluoro; or R⁶ and R⁷ are taken together with the nitrogen to which they are attached to form indolinyl, pyrrolidinyl, piperidinyl, piperazinyl, or morpholinyl;

(k) R⁸ is C₁₋₆ alkyl, C₃₋₇ cycloalkyl, C₄₋₁₀ alkylcycloalkyl, which are all optionally substituted from one to three times with halo, cyano, nitro, hydroxy, C₁₋₆ alkoxy, or phenyl; or

R⁸ is C_{6 or 10} aryl which is optionally substituted by up to three halo, cyano, nitro, hydroxy, C₃₋₇ cycloalkyl, C₄₋₁₀ alkylcycloalkyl, C₂₋₆ alkenyl, C₁₋₆ alkoxy, hydroxy-C₁₋₆ alkyl, C₁₋₆ alkyl optionally substituted with up to 5 fluoro, C₁₋₆ alkoxy optionally substituted with up to 5 fluoro; or R⁸ is C₁₋₆ alkyl optionally substituted with up to 5 fluoro groups; or R⁸ is a tetrahydrofuran ring linked through the C₃ or C₄ position of the tetrahydrofuran ring; or R⁸ is a tetrapyranyl ring linked through the C₄ position of the tetrapyranyl ring;

(l) Y is a C₅₋₇ saturated or unsaturated chain optionally containing one or two heteroatoms selected from O, S, or NR⁹R¹⁰; and

(m) R⁹ and R¹⁰ are each independently H, C₁₋₆ alkyl, C₃₋₇ cycloalkyl, C₄₋₁₀ cycloalkyl-alkyl, or substituted or unsubstituted phenyl; or R⁹ and R¹⁰ are taken together with the nitrogen to which they are attached to form indolinyl, pyrrolidinyl, piperidinyl, piperazinyl, or morpholinyl.

The present embodiments also provide compounds having the formula (XI) or a pharmaceutically acceptable salt, prodrug, or ester thereof wherein:

(a) Z is a group configured to hydrogen bond to an NS3 protease His57 imidazole moiety, and to hydrogen bond with the hydrogen and nitrogen of the backbone amide group of the NS3 amino acid at position 137;

(b) P₁′ is a group configured to form a non-polar interaction with at least one NS3 protease S1′ pocket moiety selected from the group consisting of Lys136, Gly137, Ser139, His 57, Gly58, Gln41, Ser42, and Phe43;

(g) L is a linker group consisting of from 1 to 5 atoms selected from the group consisting of carbon, oxygen, nitrogen, hydrogen, and sulfur;

(h) P₂ is selected from the group consisting of unsubstituted aryl, substituted aryl, unsubstituted heteroaryl, substituted heteroaryl, unsubstituted heterocyclic and substituted heterocyclic; P₂ being configured to form a non-polar interaction with at least one NS3 protease S2 pocket moiety selected from the group consisting of Tyr56, Gly58, Ala59, Gly60, Gln41, His57, Va178, Asp79, Gln80 and Asp81, and P₂ being configured so that no atom of P2 makes a nonpolar or polar interaction with an epsilon, zeta, or eta sidechain atom of the amino acid at position 155;

(i) R⁵ is selected from the group consisting of H, C(O)NR⁶R⁷ and C(O)OR⁸;

(j)R⁶ and R⁷ are each independently H, C₁₋₆ alkyl, C₃₋₇ cycloalkyl, C₄₋₁₀ alkylcycloalkyl or phenyl, said phenyl optionally substituted by up to three halo, cyano, nitro, hydroxy, C₃₋₇ cycloalkyl, C₄₋₁₀ alkylcycloalkyl, C₂₋₆ alkenyl, hydroxy-C₁₋₆ alkyl, C₁₋₆ alkyl optionally substituted with up to 5 fluoro, C₁₋₆ alkoxy optionally substituted with up to 5 fluoro; or R⁶ and R⁷ are taken together with the nitrogen to which they are attached to form indolinyl, pyrrolidinyl, piperidinyl, piperazinyl, or morpholinyl;

(k) R⁸ is C₁₋₆ alkyl, C₃₋₇ cycloalkyl, C₄₋₁₀ alkylcycloalkyl, which are all optionally substituted from one to three times with halo, cyano, nitro, hydroxy, C₁₋₆ alkoxy, or phenyl; or R⁸ is C_{6 or 10} aryl which is optionally substituted by up to three halo, cyano, nitro, hydroxy, C₃₋₇ cycloalkyl, C₄₋₁₀ alkylcycloalkyl, C₂₋₆ alkenyl, C₁₋₆ alkoxy, hydroxy-C₁₋₆ alkyl, C₁₋₆ alkyl optionally substituted with up to 5 fluoro, C₁₋₆ alkoxy optionally substituted with up to 5 fluoro; or R⁸ is C₁₋₆ alkyl optionally substituted with up to 5 fluoro groups; or R⁸ is a tetrahydrofuran ring linked through the C₃ or C₄ position of the tetrahydrofuran ring; or R⁸ is a tetrapyranyl ring linked through the C₄ position of the tetrapyranyl ring;

(l) Y is a C₅₋₇ saturated or unsaturated chain optionally containing one or two heteroatoms selected from O, S, or NR⁹R¹⁰; and

(m) R⁹ and R¹⁰ are each independently H, C₁₋₆ alkyl, C₃₋₇ cycloalkyl, C₄₋₁₀ cycloalkyl-alkyl, or substituted or unsubstituted phenyl; or R⁹ and R¹⁰ are taken together with the nitrogen to which they are attached to form indolinyl, pyrrolidinyl, piperidinyl, piperazinyl, or morpholinyl.

Also provided are compounds having a 50% inhibition concentration (IC₅₀) of wild-type NS3 protease of 20 nM or less. Further provided are compounds having an IC₅₀ of an NS3 protease mutated at position 155 of 200 nM or less. Also provided are compounds having both a 50% inhibition concentration (IC₅₀) of wild-type NS3 protease of 20 nM or less, and an IC₅₀ of an NS3 protease mutated at position 155 of 200 nM or less.

Also provided herein are compounds containing moieties configured to interact with particular regions, particular amino acid residues, or particular atoms of NS3 protease. Some compounds provided herein contain one or more moieties configured to form a hydrogen bond with NS3 protease at a particular region, amino acid residue, or atom. Some compounds provided herein contain one or more moieties configured to form a hydrogen bond or non-polar interaction with NS3 protease at a particular region, amino acid residue, or atom. For example, the compound having the general Formula XI may contain one or more moieties that form a hydrogen bond with a peptide backbone atom or side chain moiety located in the substrate binding pocket of NS3 protease. In another example, the compound having the general Formula XI may contain one or more moieties that form non-polar interactions with peptide backbone or side chain atom or atoms located in the substrate binding pocket of NS3 protease.

As provided in the compound having the general Formula XI, Z may be configured to form a hydrogen bond with a peptide backbone atom or side chain moiety located in the substrate binding pocket of NS3 protease, including, but not limited to, NS3 protease His57 imidazole moiety and hydrogen and nitrogen atoms of the amino acid at position 137 of NS3 protease. In some instances, Z may be configured to form a hydrogen bond with both the NS3 protease His57 imidazole moiety and hydrogen and nitrogen atoms of the amino acid at position 137 of NS3 protease.

The P1′ group of the compound having the general Formula XI may be configured to form a non-polar interaction with peptide backbone or side chain atom or atoms located in the substrate binding pocket of NS3 protease, including, but not limited to amino acid residues that form the NS3 protease S1′ pocket. For example the P1′ group may form a non-polar interaction with at least one amino acid selected from Lys136, Gly137, Ser139, His 57, Gly58, Gln41, Ser42, and Phe43.

The P₂ group of the compound having the general Formula XI may be configured to form a non-polar interaction with peptide backbone or side chain atom or atoms located in the substrate binding pocket of NS3 protease, including, but not limited to amino acid residues that form the NS3 protease S2 pocket. For example the P₂ group may form a non-polar interaction with at least one amino acid selected from Tyr56, Gly58, Ala59, Gly60, Gln41, His57, Va178, Asp79, Gln80 and Asp81. The P₂ group also may be configured to form a polar interaction with peptide backbone or side chain atom or atoms located in the substrate binding pocket of NS3 protease, including, but not limited to amino acid residues that form the NS3 protease S2 pocket. For example the P₂ group may form a polar interaction with at least one amino acid selected from Tyr56, Gly58, Ala59, Gly60, Gln41, His 57, Va178, Asp79, Gln80 and Asp81. The P₂ group also may be configured to form a hydrogen bond with peptide backbone or side chain atom or atoms located in the substrate binding pocket of NS3 protease, including, but not limited to amino acid residues that form the NS3 protease S2 pocket. For example the P₂ group may form a hydrogen bond with at least one amino acid selected from Tyr56, Gly58, Ala59, Gly60, Gln41, His57, Va178, Asp79, Gln80 and Asp81. In some instances, P₂ may form two or more of a non-polar interaction, polar interaction, and a hydrogen bond with peptide backbone or side chain moieties or atoms located in the substrate binding pocket of NS3 protease, such amino acids selected from Tyr56, Gly58, Ala59, Gly60, Gln41, His57, Va178, Asp79, Gln80 and Asp81. Such hydrogen bond, polar interaction and non-polar interaction may occur with the same amino acid residue or with different amino acid residues in the NS3 protease S2 pocket. In some embodiments, P₂ may be selected from the group consisting of unsubstituted aryl, substituted aryl, unsubstituted heteroaryl, substituted heteroaryl, unsubstituted heterocyclic and substituted heterocyclic.

The P₂ group of the compound having the general Formula XI may be configured so that no atom of P₂ makes a nonpolar or polar interaction with an epsilon, zeta, or eta sidechain atom of the amino acid at position 155. For example, the P₂ group may be configured so that no atom of P₂ makes a nonpolar or polar interaction with an epsilon, zeta, or eta sidechain atom Arg155. In another example, the P₂ group may be configured so that no atom of P₂ makes a nonpolar or polar interaction with an epsilon, zeta, or eta sidechain atom of a non-arginine amino acid at 155. Examples of non-arginine amino acids at 155 include Lys155 and Gln155.

As provided in the compound having the general Formula XI, L may be a linker group that links P₂ to the heterocyclic backbone of the compound of Formula XI. Linker L may contain any of a variety of atoms and moieties suitable for positioning P₂ in the NS3 protease substrate binding pocket. In one embodiment, L may contain 1 to 5 atoms selected from the group consisting of carbon, oxygen, nitrogen, hydrogen, and sulfur. In another embodiment, L may contain 2 to 5 atoms selected from the group consisting of carbon, oxygen, nitrogen, hydrogen, and sulfur. For example, L may contain a group having the formula —W—C(═V)—, where V and W are each individually selected from O, S or NH. Specific exemplary groups for L include, but are not limited to, ester, amide, carbamate, thioester, and thioamide.

The compound of Formula XI also may contain an R⁵ group, where the R⁵ group may contain a carboxyl moiety. Exemplary carboxyl moieties of R⁵ include C(O)NR⁶R⁷ and C(O)OR⁸ where R⁶ and R⁷ are each independently H, C₁₋₆ alkyl, C₃₋₇ cycloalkyl, C₄₋₁₀ alkylcycloalkyl or phenyl, said phenyl optionally substituted by up to three halo, cyano, nitro, hydroxy, C₃₋₇ cycloalkyl, C₄₋₁₀ alkylcycloalkyl, C₂₋₆ alkenyl, hydroxy-C₁₋₆ alkyl, C₁₋₆ alkyl optionally substituted with up to 5 fluoro, C₁₋₆ alkoxy optionally substituted with up to 5 fluoro; or R⁶ and R⁷ are taken together with the nitrogen to which they are attached to form indolinyl, pyrrolidinyl, piperidinyl, piperazinyl, or morpholinyl; and where R⁸ is C₁₋₆ alkyl, C₃₋₇ cycloalkyl, C₄₋₁₀ alkylcycloalkyl, which are all optionally substituted from one to three times with halo, cyano, nitro, hydroxy, C₁₋₆ alkoxy, or phenyl; or R⁸ is C_{6 or 10} aryl which is optionally substituted by up to three halo, cyano, nitro, hydroxy, C₃₋₇ cycloalkyl, C₄₋₁₀ alkylcycloalkyl, C₂₋₆ alkenyl, C₁₋₆ alkoxy, hydroxy-C₁₋₆ alkyl, C₁₋₆ alkyl optionally substituted with up to 5 fluoro, C₁₋₆ alkoxy optionally substituted with up to 5 fluoro; or R⁸ is C₁₋₆ alkyl optionally substituted with up to 5 fluoro groups; or R⁸ is a tetrahydrofuran ring linked through the C₃ or C₄ position of the tetrahydrofuran ring; or R⁸ is a tetrapyranyl ring linked through the C₄ position of the tetrapyranyl ring.

### Formula XII

The present embodiments provide compounds having the formula XII:

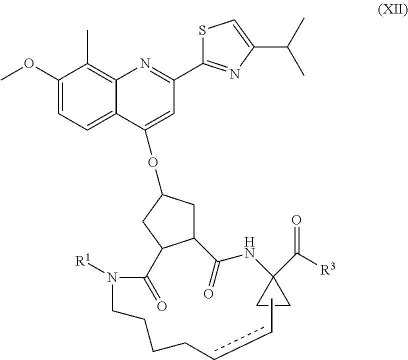

or a pharmaceutically acceptable salt or prodrug thereof, wherein is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NHC(O)NR^{1a}R^{1b}, —C(O)OR^{1c}, and heteroaryl; in some embodiments, said heteroaryl may contain 1-3 heteroatoms independently selected from N or O.

R^{1e} is selected from the group consisting of t-butyl, cycloalkyl, and heterocyclyl; R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl, wherein in some embodiments, said heteroaryl may contain 1-3 heteroatoms independently selected from N or O; and R^{1c} and R^{1d} are each separately selected from the group consisting of —H, C₁₋₄ alkoxy, C₁₋₆ alkyl, C₃₋₇ cycloalkyl, aryl, arylalkyl and heteroaryl.

R³ is —OH, —NHS(O)₂R^{3a}, —NHS(O)₂OR^{3a} or —NHS(O)₂NR^{3b}R^{3c}; where R^{1a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro.

R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{3b} and R^{3c} together with N form a three- to six-membered heterocyclic ring, bonded to the parent structure through a nitrogen, and the heterocylic ring is optionally substituted with one or more substituents each independently selected from the group consisting of halo, cyano, nitro, C₁₋₆ alkyl, C₁₋₆ alkoxy, and phenyl.

Each t is independently 0, 1 or 2; each q is independently 0, 1 or 2; and any bond represented by a dashed and solid line represents a bond selected from the group consisting of a single bond and a double bond.

In some embodiments, R¹ may be selected from —C(O)O-t-butyl or aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NHC(O)NR^{1a}R^{1b}, —C(O)OR^{1c}, and heteroaryl; in some embodiments, said heteroaryl may contain 1-3 heteroatoms independently selected from N or O.

In some embodiments, compounds of Formula I have the structure of Formula XIIa:

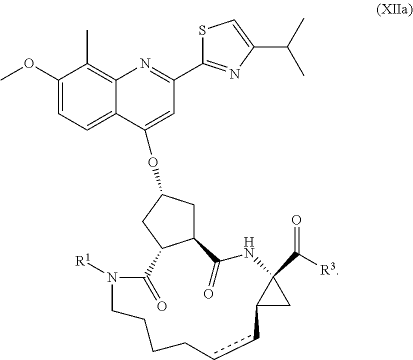

In one embodiments, the compound of formula XII is:

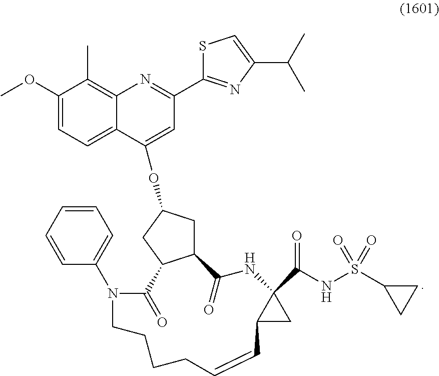

Some embodiments provide a compound having the structure of Formula I or XII:

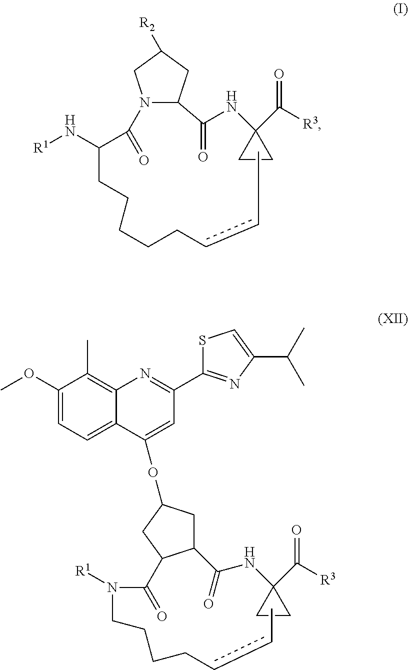

or a pharmaceutically acceptable salt or prodrug thereof wherein: R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NHC(O)NR^{1a}R^{1b}, —C(O)OR^{1c}, and heteroaryl containing 1-3 heteroatoms independently selected from N or O.

R^{1e} is selected from the group consisting of t-butyl, cycloalkyl, and heterocyclyl containing 1-3 heteroatoms independently selected from N, O and S. R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl containing 1-3 heteroatoms independently selected from N and O R^{1c} and R^{1d} are each separately selected from the group consisting of —H, C₁₋₄ alkoxy, linear and branched C₁₋₆ alkyl, C₃₋₇ cycloalkyl, aryl, arylalkyl and heteroaryl containing 1-3 heteroatoms independently selected from N, O and S.

R² is selected from the group consisting of

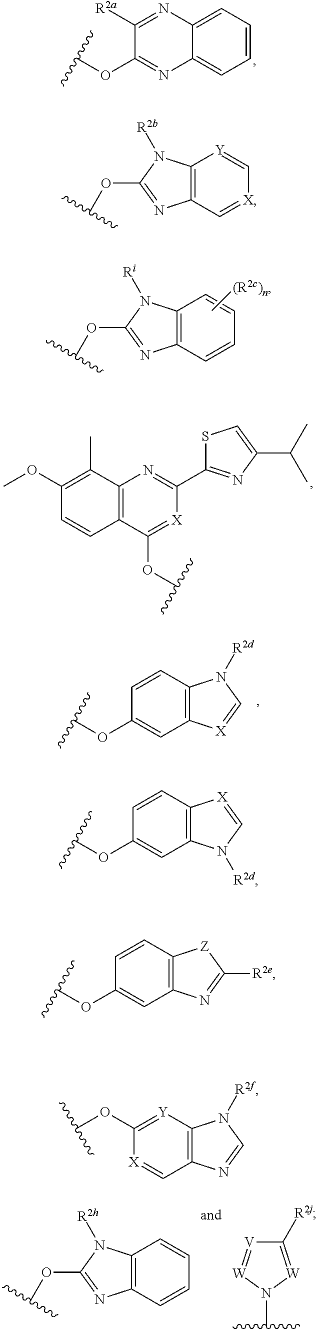

wherein X and Y are each independently selected from —CH— or —N—, wherein X and Y are not both —CH—; Z is O or S; V and W are each independently selected from —CR^{2k}— or —N—, wherein V and W are not both —CR^{2k}—; n is 1, 2 or 3.

R^{2j} and R^{2k} are each independently selected from the group consisting of H, halo, optionally substituted aryl, optionally substituted heteroaryl containing 1-3 heteroatoms independently selected from S, N or O; or R^{2j} and R^{2k} together form an aryl ring optionally substituted by 1-3 R².

R^{2a}, each R^{2c}, R^{2e} and R^{2g} are each independently selected from the group consisting of halo, —C(O)OR^{1c}, —C(O)NR′R″, —NR′R″, —NHC(O)NR′R″, —NHC(O)OR^{1c}, —NHS(O)₂R^{1c}, linear and branched C₁₋₆ alkyl optionally substituted with up to 5 fluoro, C₂₋₆ alkenyl, C₃₋₇ cycloalkyl, optionally substituted C₁₋₆ alkoxy, optionally substituted aryl and optionally substituted heteroaryl containing 1-3 heteroatoms independently selected from S, N or O.

R^{2b}, R^{2d} and R^{2f} are each independently selected from the group consisting of linear and branched C₁₋₆ alkyl optionally substituted with up to 5 fluoro, C₂₋₆ alkenyl, C₃₋₇ cycloalkyl, arylalkyl, optionally substituted aryl and optionally substituted heteroaryl containing 1-3 heteroatoms independently selected from S, N or O.

R^{2h} is selected from the group consisting of propyl, butyl and phenyl; Rⁱ is C₁₋₆ alkyl optionally substituted with up to 5 fluoro; R′ and R″ are each independently selected from the group consisting of —H, optionally substituted linear and branched C₁₋₆ alkyl, optionally substituted C₂₋₆ alkenyl, optionally substituted aryl, optionally substituted arylalkyl and optionally substituted heteroaryl containing 1-3 heteroatoms independently selected from S, N or O.

R³ is —OH, —NHS(O)₂R^{3a}, —NHS(O)₂OR^{3a} or —NHS(O)₂NR^{3b}R^{3c}; where R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro. R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{3b} and R^{3c} are taken together with the nitrogen to which they are attached to form a three- to six-membered heterocylic ring containing 1-3 heteroatoms independently selected from S, N or O, and the heterocylic ring is optionally substituted with one or more substituents each independently selected from the group consisting of halo, cyano, nitro, C₁₋₆ alkyl, C₁₋₆ alkoxy, and phenyl. Each t is independently 0, 1 or 2; and each q is independently 0, 1 or 2.

Any bond represented by a dashed and solid line represents a bond selected from the group consisting of a single bond and a double bond.

Provided that if R² is

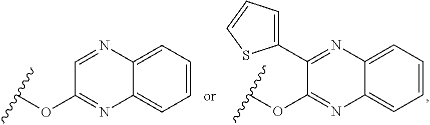

then R¹ is not phenyl.

Provided that if R² is

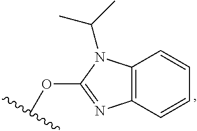

then R¹ is not —C(O)O-t-butyl, phenyl or phenyl substituted with one or more substituents selected from the group consisting of fluoro, chloro and —CF₃.

Provided that if R² is

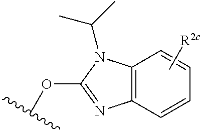

and R^{2c} is —F or methyl, then R¹ is not —C(O)O-t-butyl or phenyl.

Provided that if R² is

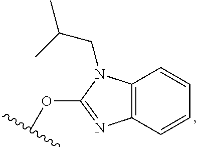

then R¹ is not —C(O)O-t-butyl or phenyl substituted with one or more substituents selected from the group consisting of fluoro and —CF₃.

Provided that if R² is

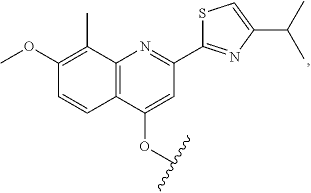

then R¹ is not —C(O)O-t-butyl, benzoxazyl, t-butylthiazyl, phenyl or phenyl substituted with one or more substituents selected from the group consisting of fluoro, chloro, methyl, —CF₃ and —OCF₃.

### Salts and Other Compounds

Some embodiments provide a compound selected from the group consisting of:

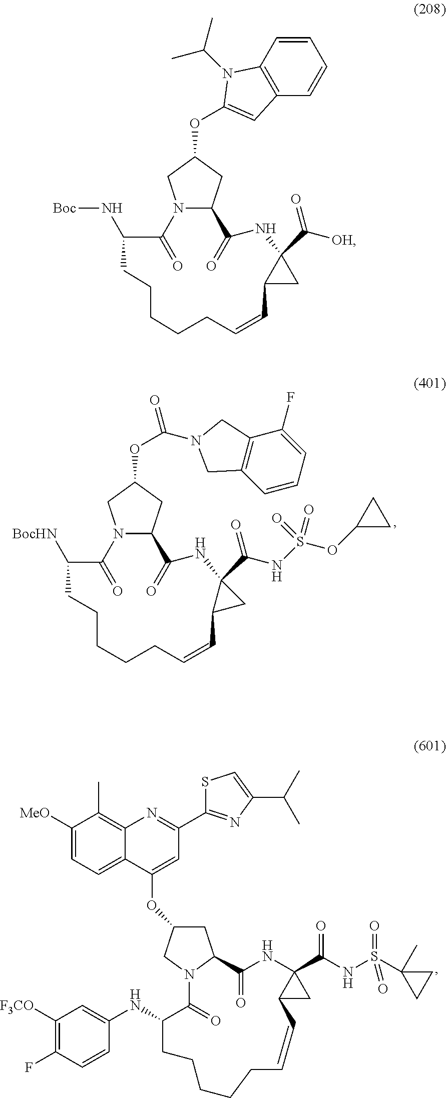

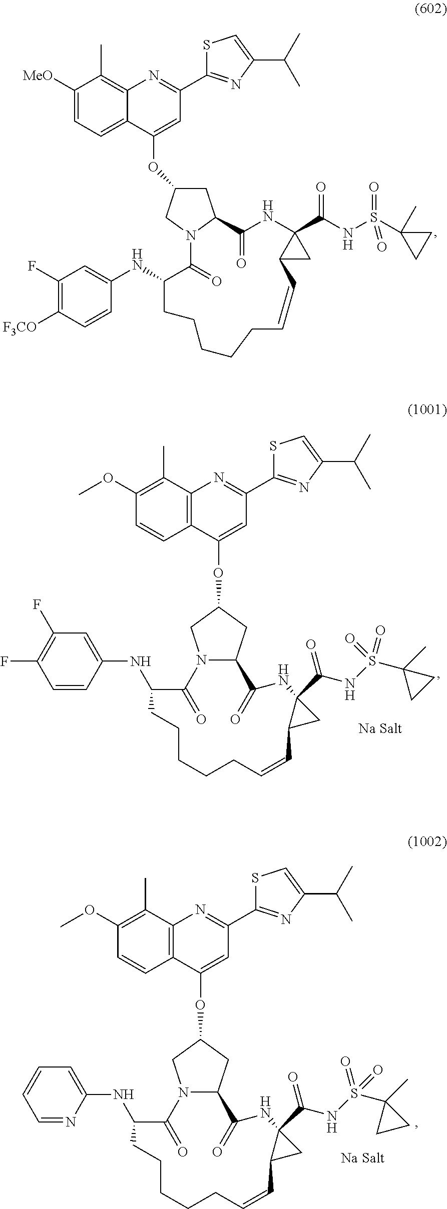

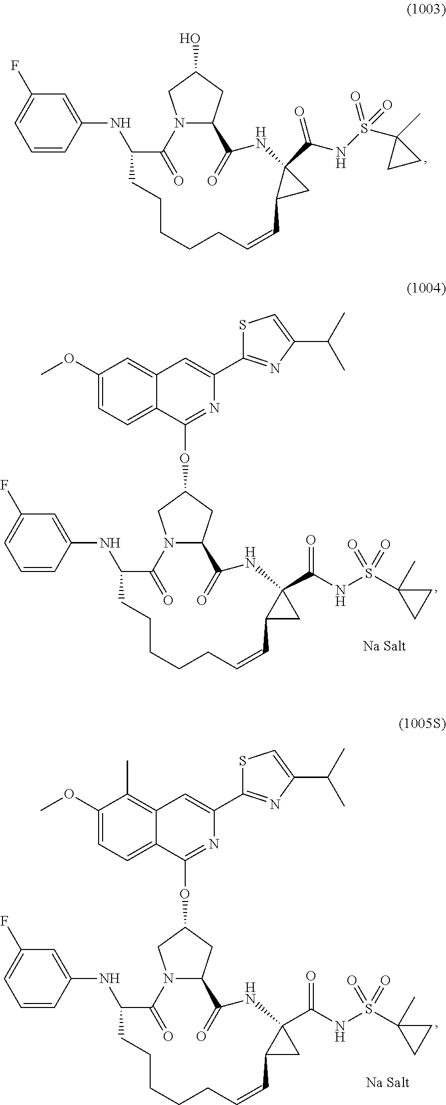

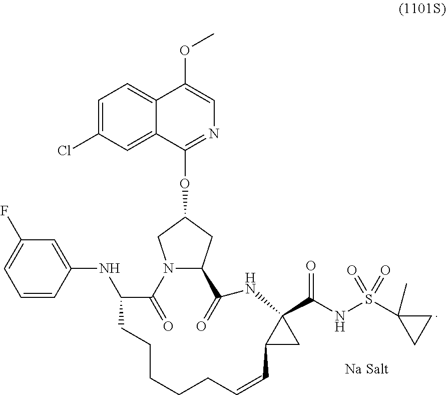

For any of the above formulas, in some embodiments, C₁₋₆ alkyl may include linear and branched C₁₋₆ alkyl, and C₁₋₆ alkoxy may include linear and branched C₁₋₆ alkoxy.

### Compositions

The present embodiments further provide compositions, including pharmaceutical compositions, comprising compounds of the general Formulae I, Ia, II, III, IV, V, VI-1, VI-2, VII, VIII, IX, X, XI, and XII, or any compounds disclosed herein.

A subject pharmaceutical composition comprises a subject compound; and a pharmaceutically acceptable excipient. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

The present embodiments provide for a method of inhibiting NS3/NS4 protease activity comprising contacting a NS3/NS4 protease with a compound disclosed herein.

The present embodiments provide for a method of treating hepatitis by modulating NS3/NS4 protease comprising contacting a NS3/NS4 protease with a compound disclosed herein.

Example compounds of Formulae I, Ia, II, III, IV, V, VI-1, VI-2, VII, VIII, IX, X, XI, and XII include Compound Numbers 101-129, 200-299, 301-312, 401, 501-506, 601-602, 701-702, 801-805, 901, 1001-1003, 1102-1103, 1201-1224, 1251-1253, 1401-1436, and 1701-1780 as set forth herein. In addition, Compounds 401, 1004, 1005, 1005S, 1101, 1101S are also disclosed.

Preferred embodiments provide a method of treating a hepatitis C virus infection in an individual, the method comprising administering to the individual an effective amount of a composition comprising a preferred compound.

Preferred embodiments provide a method of treating liver fibrosis in an individual, the method comprising administering to the individual an effective amount of a composition comprising a preferred compound.

Preferred embodiments provide a method of increasing liver function in an individual having a hepatitis C virus infection, the method comprising administering to the individual an effective amount of a composition comprising a preferred compound.

In many embodiments, a subject compound inhibits the enzymatic activity of a hepatitis virus C(HCV) NS3 protease. Whether a subject compound inhibits HCV NS3 protease can be readily determined using any known method. Typical methods involve a determination of whether an HCV polyprotein or other polypeptide comprising an NS3 recognition site is cleaved by NS3 in the presence of the agent. In many embodiments, a subject compound inhibits NS3 enzymatic activity by at least about 10%, at least about 15%, at least about 20%, at least about 25%, at least about 30%, at least about 40%, at least about 50%, at least about 60%, at least about 70%, at least about 80%, or at least about 90%, or more, compared to the enzymatic activity of NS3 in the absence of the compound.

In many embodiments, a subject compound inhibits enzymatic activity of an HCV NS3 protease with an IC₅₀ of less than about 50 μM, e.g., a subject compound inhibits an HCV NS3 protease with an IC₅₀ of less than about 40 μM, less than about 25 μM, less than about 10 μM, less than about 1 μM, less than about 100 nM, less than about 80 nM, less than about 60 nM, less than about 50 nM, less than about 25 nM, less than about 10 nM, less than about 5 nM, less than about 1 nM, or less than about 0.5 nM, or less.

In many embodiments, a subject compound inhibits the enzymatic activity of a hepatitis virus C(HCV) NS3 helicase. Whether a subject compound inhibits HCV NS3 helicase can be readily determined using any known method. In many embodiments, a subject compound inhibits NS3 enzymatic activity by at least about 10%, at least about 15%, at least about 20%, at least about 25%, at least about 30%, at least about 40%, at least about 50%, at least about 60%, at least about 70%, at least about 80%, or at least about 90%, or more, compared to the enzymatic activity of NS3 in the absence of the compound.

In many embodiments, a subject compound inhibits HCV viral replication. For example, a subject compound inhibits HCV viral replication by at least about 10%, at least about 15%, at least about 20%, at least about 25%, at least about 30%, at least about 40%, at least about 50%, at least about 60%, at least about 70%, at least about 80%, or at least about 90%, or more, compared to HCV viral replication in the absence of the compound. Whether a subject compound inhibits HCV viral replication can be determined using methods known in the art, including an in vitro viral replication assay.

### Treating a Hepatitis Virus Infection

The methods and compositions described herein are generally useful in treatment of an of HCV infection.

Whether a subject method is effective in treating an HCV infection can be determined by a reduction in viral load, a reduction in time to seroconversion (virus undetectable in patient serum), an increase in the rate of sustained viral response to therapy, a reduction of morbidity or mortality in clinical outcomes, or other indicator of disease response.

In general, an effective amount of a compound of Formulae I, Ia, II, III, IV, V, VI-1, VI-2, VII, VIII, IX, X, XI, or, XII, or any compounds disclosed herein, and optionally one or more additional antiviral agents, is an amount that is effective to reduce viral load or achieve a sustained viral response to therapy.

Whether a subject method is effective in treating an HCV infection can be determined by measuring viral load, or by measuring a parameter associated with HCV infection, including, but not limited to, liver fibrosis, elevations in serum transaminase levels, and necroinflammatory activity in the liver. Indicators of liver fibrosis are discussed in detail below.

The method involves administering an effective amount of a compound of Formulae I, Ia, II, III, IV, V, VI-1, VI-2, VII, VIII, IX, X, XI, or, XII, or any compounds disclosed herein, optionally in combination with an effective amount of one or more additional antiviral agents. In some embodiments, an effective amount of a compound of Formulae I, Ia, II, III, IV, V, VI-1, VI-2, VII, VIII, IX, X, XI, or, XII, or any compounds disclosed herein, and optionally one or more additional antiviral agents, is an amount that is effective to reduce viral titers to undetectable levels, e.g., to about 1000 to about 5000, to about 500 to about 1000, or to about 100 to about 500 genome copies/mL serum. In some embodiments, an effective amount of a compound of Formulae I, Ia, II, III, IV, V, VI-1, VI-2, VII, VIII, IX, X, XI, or, XII, or any compounds disclosed herein, and optionally one or more additional antiviral agents, is an amount that is effective to reduce viral load to lower than 100 genome copies/mL serum.

In some embodiments, an effective amount of a compound of Formulae I, Ia, II, III, IV, V, VI-1, VI-2, VII, VIII, IX, X, XI, or, XII, or any compounds disclosed herein, and optionally one or more additional antiviral agents, is an amount that is effective to achieve a 1.5-log, a 2-log, a 2.5-log, a 3-log, a 3.5-log, a 4-log, a 4.5-log, or a 5-log reduction in viral titer in the serum of the individual.

In many embodiments, an effective amount of a compound of Formulae I, Ia, II, III, IV, V, VI-1, VI-2, VII, VIII, IX, X, XI, or, XII, or any compounds disclosed herein, and optionally one or more additional antiviral agents, is an amount that is effective to achieve a sustained viral response, e.g., non-detectable or substantially non-detectable HCV RNA (e.g., less than about 500, less than about 400, less than about 200, or less than about 100 genome copies per milliliter serum) is found in the patient's serum for a period of at least about one month, at least about two months, at least about three months, at least about four months, at least about five months, or at least about six months following cessation of therapy.

As noted above, whether a subject method is effective in treating an HCV infection can be determined by measuring a parameter associated with HCV infection, such as liver fibrosis. Methods of determining the extent of liver fibrosis are discussed in detail below. In some embodiments, the level of a serum marker of liver fibrosis indicates the degree of liver fibrosis.

As one non-limiting example, levels of serum alanine aminotransferase (ALT) are measured, using standard assays. In general, an ALT level of less than about 45 international units is considered normal. In some embodiments, an effective amount of a compound of Formulae I, Ia, II, III, IV, V, VI-1, VI-2, VII, VIII, IX, X, XI, or, XII, or any compounds disclosed herein, and optionally one or more additional antiviral agents, is an amount effective to reduce ALT levels to less than about 45 IU/mL serum.

A therapeutically effective amount of a compound of Formulae I, Ia, II, III, IV, V, VI-1, VI-2, VII, VIII, IX, X, XI, or, XII, or any compounds disclosed herein, and optionally one or more additional antiviral agents, is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Methods of measuring serum markers include immunological-based methods, e.g., enzyme-linked immunosorbent assays (ELISA), radioimmunoassays, and the like, using antibody specific for a given serum marker.

In many embodiments, an effective amount of a compound of Formulae I, Ia, II, III, IV, V, VI-1, VI-2, VII, VIII, IX, X, XI, or, XII, or any compounds disclosed herein and an additional antiviral agent is a synergistic amount. The additional antiviral agent may itself be a combination of antiviral agents, e.g., a combination of pegylated interferon-alfa and ribavirin. As used herein, a “synergistic combination” or a “synergistic amount” of a compound of Formulae I, Ia, II, III, IV, V, VI-1, VI-2, VII, VIII, IX, X, XI, or, XII, or any compounds disclosed herein and an additional antiviral agent is a combined dosage that is more effective in the therapeutic or prophylactic treatment of an HCV infection than the incremental improvement in treatment outcome that could be predicted or expected from a merely additive combination of (i) the therapeutic or prophylactic benefit of the compound of Formulae I, Ia, II, III, IV, V, VI-1, VI-2, VII, VIII, IX, X, XI, or, XII, or any compounds disclosed herein when administered at that same dosage as a monotherapy and (ii) the therapeutic or prophylactic benefit of the additional antiviral agent when administered at the same dosage as a monotherapy.

In some embodiments, a selected amount of a compound of Formulae I, Ia, II, III, IV, V, VI-1, VI-2, VII, VIII, IX, X, XI, or, XII, or any compounds disclosed herein and a selected amount of an additional antiviral agent are effective when used in combination therapy for a disease, but the selected amount of the compound of Formulae I, Ia, II, III, IV, V, VI-1, VI-2, VII, VIII, IX, X, XI, or, XII, or any compounds disclosed herein and/or the selected amount of the additional antiviral agent is ineffective when used in monotherapy for the disease. Thus, the embodiments encompass (1) regimens in which a selected amount of the additional antiviral agent enhances the therapeutic benefit of a selected amount of the compound of Formulae I, Ia, II, III, IV, V, VI-1, VI-2, VII, VIII, IX, X, XI, or, XII, or any compounds disclosed herein when used in combination therapy for a disease, where the selected amount of the additional antiviral agent provides no therapeutic benefit when used in monotherapy for the disease (2) regimens in which a selected amount of the compound of Formulae I, Ia, II, III, IV, V, VI-1, VI-2, VII, VIII, IX, X, XI, or, XII, or any compounds disclosed herein enhances the therapeutic benefit of a selected amount of the additional antiviral agent when used in combination therapy for a disease, where the selected amount of the compound of Formulae I, Ia, II, III, IV, V, VI-1, VI-2, VII, VIII, IX, X, XI, or, XII, or any compounds disclosed herein provides no therapeutic benefit when used in monotherapy for the disease and (3) regimens in which a selected amount of the compound of Formulae I, Ia, II, III, IV, V, VI-1, VI-2, VII, VIII, IX, X, XI, or, XII, or any compounds disclosed herein and a selected amount of the additional antiviral agent provide a therapeutic benefit when used in combination therapy for a disease, where each of the selected amounts of the compound of Formulae I, Ia, II, III, IV, V, VI-1, VI-2, VII, VIII, IX, X, XI, or, XII, or any compounds disclosed herein and the additional antiviral agent, respectively, provides no therapeutic benefit when used in monotherapy for the disease. As used herein, a “synergistically effective amount” of a compound of Formulae I, Ia, II, III, IV, V, VI-1, VI-2, VII, VIII, IX, X, XI, or, XII, or any compounds disclosed herein and an additional antiviral agent, and its grammatical equivalents, shall be understood to include any regimen encompassed by any of (1)-(3) above.

### Fibrosis

The embodiments provides methods for treating liver fibrosis (including forms of liver fibrosis resulting from, or associated with, HCV infection), generally involving administering a therapeutic amount of a compound of Formulae I, Ia, II, III, IV, V, VI-1, VI-2, VII, VIII, IX, X, XI, or, XII, or any compounds disclosed herein, and optionally one or more additional antiviral agents. Effective amounts of compounds of Formulae I, Ia, II, III, IV, V, VI-1, VI-2, VII, VIII, IX, X, XI, or, XII, or any compounds disclosed herein, with and without one or more additional antiviral agents, as well as dosing regimens, are as discussed below.

Whether treatment with a compound of Formulae I, Ia, II, III, IV, V, VI-1, VI-2, VII, VIII, IX, X, XI, or, XII, or any compounds disclosed herein, and optionally one or more additional antiviral agents, is effective in reducing liver fibrosis is determined by any of a number of well-established techniques for measuring liver fibrosis and liver function. Liver fibrosis reduction is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems.

The METAVIR scoring system is based on an analysis of various features of a liver biopsy, including fibrosis (portal fibrosis, centrilobular fibrosis, and cirrhosis); necrosis (piecemeal and lobular necrosis, acidophilic retraction, and ballooning degeneration); inflammation (portal tract inflammation, portal lymphoid aggregates, and distribution of portal inflammation); bile duct changes; and the Knodell index (scores of periportal necrosis, lobular necrosis, portal inflammation, fibrosis, and overall disease activity). The definitions of each stage in the METAVIR system are as follows: score: 0, no fibrosis; score: 1, stellate enlargement of portal tract but without septa formation; score: 2, enlargement of portal tract with rare septa formation; score: 3, numerous septa without cirrhosis; and score: 4, cirrhosis.

Knodell's scoring system, also called the Hepatitis Activity Index, classifies specimens based on scores in four categories of histologic features: I. Periportal and/or bridging necrosis; II. Intralobular degeneration and focal necrosis; III. Portal inflammation; and IV. Fibrosis. In the Knodell staging system, scores are as follows: score: 0, no fibrosis; score: 1, mild fibrosis (fibrous portal expansion); score: 2, moderate fibrosis; score: 3, severe fibrosis (bridging fibrosis); and score: 4, cirrhosis. The higher the score, the more severe the liver tissue damage. Knodell (1981) Hepatol. 1:431.

In the Scheuer scoring system scores are as follows: score: 0, no fibrosis; score: 1, enlarged, fibrotic portal tracts; score: 2, periportal or portal-portal septa, but intact architecture; score: 3, fibrosis with architectural distortion, but no obvious cirrhosis; score: 4, probable or definite cirrhosis. Scheuer (1991) J. Hepatol. 13:372.

The Ishak scoring system is described in Ishak (1995) J. Hepatol. 22:696-699. Stage 0, No fibrosis; Stage 1, Fibrous expansion of some portal areas, with or without short fibrous septa; stage 2, Fibrous expansion of most portal areas, with or without short fibrous septa; stage 3, Fibrous expansion of most portal areas with occasional portal to portal (P-P) bridging; stage 4, Fibrous expansion of portal areas with marked bridging (P-P) as well as portal-central (P-C); stage 5, Marked bridging (P-P and/or P-C) with occasional nodules (incomplete cirrhosis); stage 6, Cirrhosis, probable or definite.

The benefit of anti-fibrotic therapy can also be measured and assessed by using the Child-Pugh scoring system which comprises a multicomponent point system based upon abnormalities in serum bilirubin level, serum albumin level, prothrombin time, the presence and severity of ascites, and the presence and severity of encephalopathy. Based upon the presence and severity of abnormality of these parameters, patients may be placed in one of three categories of increasing severity of clinical disease: A, B, or C.

In some embodiments, a therapeutically effective amount of a compound of Formulae I, Ia, II, III, IV, V, VI-1, VI-2, VII, VIII, IX, X, XI, or, XII, or any compounds disclosed herein, and optionally one or more additional antiviral agents, is an amount that effects a change of one unit or more in the fibrosis stage based on pre- and post-therapy liver biopsies. In particular embodiments, a therapeutically effective amount of a compound of Formulae I, Ia, II, III, IV, V, VI-1, VI-2, VII, VIII, IX, X, XI, or, XII, or any compounds disclosed herein, and optionally one or more additional antiviral agents, reduces liver fibrosis by at least one unit in the METAVIR, the Knodell, the Scheuer, the Ludwig, or the Ishak scoring system.

Secondary, or indirect, indices of liver function can also be used to evaluate the efficacy of treatment with a compound of Formulae I, Ia, II, III, IV, V, VI-1, VI-2, VII, VIII, IX, X, XI, or, XII, or any compounds disclosed herein. Morphometric computerized semi-automated assessment of the quantitative degree of liver fibrosis based upon specific staining of collagen and/or serum markers of liver fibrosis can also be measured as an indication of the efficacy of a subject treatment method. Secondary indices of liver function include, but are not limited to, serum transaminase levels, prothrombin time, bilirubin, platelet count, portal pressure, albumin level, and assessment of the Child-Pugh score.

An effective amount of a compound of Formulae I, Ia, II, III, IV, V, VI-1, VI-2, VII, VIII, IX, X, XI, or, XII, or any compounds disclosed herein, and optionally one or more additional antiviral agents, is an amount that is effective to increase an index of liver function by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the index of liver function in an untreated individual, or to a placebo-treated individual. Those skilled in the art can readily measure such indices of liver function, using standard assay methods, many of which are commercially available, and are used routinely in clinical settings.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

A therapeutically effective amount of a compound of Formulae I, Ia, II, III, IV, V, VI-1, VI-2, VII, VIII, IX, X, XI, or, XII, or any compounds disclosed herein, and optionally one or more additional antiviral agents, is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Those skilled in the art can readily measure such serum markers of liver fibrosis, using standard assay methods, many of which are commercially available, and are used routinely in clinical settings. Methods of measuring serum markers include immunological-based methods, e.g., enzyme-linked immunosorbent assays (ELISA), radioimmunoassays, and the like, using antibody specific for a given serum marker.

Quantitative tests of functional liver reserve can also be used to assess the efficacy of treatment with an interferon receptor agonist and pirfenidone (or a pirfenidone analog). These include: indocyanine green clearance (ICG), galactose elimination capacity (GEC), aminopyrine breath test (ABT), antipyrine clearance, monoethylglycine-xylidide (MEG-X) clearance, and caffeine clearance.

As used herein, a “complication associated with cirrhosis of the liver” refers to a disorder that is a sequellae of decompensated liver disease, i.e., or occurs subsequently to and as a result of development of liver fibrosis, and includes, but it not limited to, development of ascites, variceal bleeding, portal hypertension, jaundice, progressive liver insufficiency, encephalopathy, hepatocellular carcinoma, liver failure requiring liver transplantation, and liver-related mortality.

A therapeutically effective amount of a compound of Formulae I, Ia, II, III, IV, V, VI-1, VI-2, VII, VIII, IX, X, XI, or, XII, or any compounds disclosed herein, and optionally one or more additional antiviral agents, is an amount that is effective in reducing the incidence (e.g., the likelihood that an individual will develop) of a disorder associated with cirrhosis of the liver by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to an untreated individual, or to a placebo-treated individual.

Whether treatment with a compound of Formulae I, Ia, II, III, IV, V, VI-1, VI-2, VII, VIII, IX, X, XI, or, XII, or any compounds disclosed herein, and optionally one or more additional antiviral agents, is effective in reducing the incidence of a disorder associated with cirrhosis of the liver can readily be determined by those skilled in the art.

Reduction in liver fibrosis increases liver function. Thus, the embodiments provide methods for increasing liver function, generally involving administering a therapeutically effective amount of a compound of Formulae I, Ia, II, III, IV, V, VI-1, VI-2, VII, VIII, IX, X, XI, or, XII, or any compounds disclosed herein, and optionally one or more additional antiviral agents. Liver functions include, but are not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

Whether a liver function is increased is readily ascertainable by those skilled in the art, using well-established tests of liver function. Thus, synthesis of markers of liver function such as albumin, alkaline phosphatase, alanine transaminase, aspartate transaminase, bilirubin, and the like, can be assessed by measuring the level of these markers in the serum, using standard immunological and enzymatic assays. Splanchnic circulation and portal hemodynamics can be measured by portal wedge pressure and/or resistance using standard methods. Metabolic functions can be measured by measuring the level of ammonia in the serum.

Whether serum proteins normally secreted by the liver are in the normal range can be determined by measuring the levels of such proteins, using standard immunological and enzymatic assays. Those skilled in the art know the normal ranges for such serum proteins. The following are non-limiting examples. The normal level of alanine transaminase is about 45 IU per milliliter of serum. The normal range of aspartate transaminase is from about 5 to about 40 units per liter of serum. Bilirubin is measured using standard assays. Normal bilirubin levels are usually less than about 1.2 mg/dL. Serum albumin levels are measured using standard assays. Normal levels of serum albumin are in the range of from about 35 to about 55 g/L. Prolongation of prothrombin time is measured using standard assays. Normal prothrombin time is less than about 4 seconds longer than control.

A therapeutically effective amount of a compound of Formulae I, Ia, II, III, IV, V, VI-1, VI-2, VII, VIII, IX, X, XI, or, XII, or any compounds disclosed herein, and optionally one or more additional antiviral agents, is one that is effective to increase liver function by at least about 10%, at least about 20%, at least about 30%, at least about 40%, at least about 50%, at least about 60%, at least about 70%, at least about 80%, or more. For example, a therapeutically effective amount of a compound of Formulae I, Ia, II, III, IV, V, VI-1, VI-2, VII, VIII, IX, X, XI, or, XII, or any compounds disclosed herein, and optionally one or more additional antiviral agents, is an amount effective to reduce an elevated level of a serum marker of liver function by at least about 10%, at least about 20%, at least about 30%, at least about 40%, at least about 50%, at least about 60%, at least about 70%, at least about 80%, or more, or to reduce the level of the serum marker of liver function to within a normal range. A therapeutically effective amount of a compound of Formulae I, Ia, II, III, IV, V, VI-1, VI-2, VII, VIII, IX, X, XI, or, XII, or any compounds disclosed herein, and optionally one or more additional antiviral agents, is also an amount effective to increase a reduced level of a serum marker of liver function by at least about 10%, at least about 20%, at least about 30%, at least about 40%, at least about 50%, at least about 60%, at least about 70%, at least about 80%, or more, or to increase the level of the serum marker of liver function to within a normal range.

### Dosages, Formulations, and Routes of Administration

In the subject methods, the active agent(s) (e.g., compound of Formulae I, Ia, II, III, IV, V, VI-1, VI-2, VII, VIII, IX, X, XI, or, XII, or any compounds disclosed herein, and optionally one or more additional antiviral agents) may be administered to the host using any convenient means capable of resulting in the desired therapeutic effect. Thus, the agent can be incorporated into a variety of formulations for therapeutic administration. More particularly, the agents of the embodiments can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

### Formulations

The above-discussed active agent(s) can be formulated using well-known reagents and methods. Compositions are provided in formulation with a pharmaceutically acceptable excipient(s). A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy,” 20^{th} edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, an agent is formulated in an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

As such, administration of the agents can be achieved in various ways, including oral, buccal, rectal, parenteral, intraperitoneal, intradermal, subcutaneous, intramuscular, transdermal, intratracheal, etc., administration. In many embodiments, administration is by bolus injection, e.g., subcutaneous bolus injection, intramuscular bolus injection, and the like.

The pharmaceutical compositions of the embodiments can be administered orally, parenterally or via an implanted reservoir. Oral administration or administration by injection is preferred.

Subcutaneous administration of a pharmaceutical composition of the embodiments is accomplished using standard methods and devices, e.g., needle and syringe, a subcutaneous injection port delivery system, and the like. See, e.g., U.S. Pat. Nos. 3,547,119; 4,755,173; 4,531,937; 4,311,137; and 6,017,328. A combination of a subcutaneous injection port and a device for administration of a pharmaceutical composition of the embodiments to a patient through the port is referred to herein as “a subcutaneous injection port delivery system.” In many embodiments, subcutaneous administration is achieved by bolus delivery by needle and syringe.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Furthermore, the agents can be made into suppositories by mixing with a variety of bases such as emulsifying bases or water-soluble bases. The compounds of the embodiments can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the embodiments calculated in an amount sufficient to produce the desired effect in association with a pharmaceutically acceptable diluent, carrier or vehicle. The specifications for the novel unit dosage forms of the embodiments depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

### Other Antiviral or Antifibrotic Agents

As discussed above, a subject method will in some embodiments be carried out by administering an NS3 inhibitor that is a compound of Formulae I, Ia, II, III, IV, V, VI-1, VI-2, VII, VIII, IX, X, XI, or, XII, or any compounds disclosed herein, and optionally one or more additional antiviral agent(s).

In some embodiments, the method further includes administration of one or more interferon receptor agonist(s). Interferon receptor agonists are described herein.

In other embodiments, the method further includes administration of pirfenidone or a pirfenidone analog. Pirfenidone and pirfenidone analogs are described herein.

Additional antiviral agents that are suitable for use in combination therapy include, but are not limited to, nucleotide and nucleoside analogs. Non-limiting examples include azidothymidine (AZT) (zidovudine), and analogs and derivatives thereof; 2′,3′-dideoxyinosine (DDI) (didanosine), and analogs and derivatives thereof; 2′,3′-dideoxycytidine (DDC) (dideoxycytidine), and analogs and derivatives thereof; 2′,3′-didehydro-2′,3′-dideoxythymidine (D4T) (stavudine), and analogs and derivatives thereof; combivir; abacavir; adefovir dipoxil; cidofovir; ribavirin; ribavirin analogs; and the like.

In some embodiments, the method further includes administration of ribavirin. Ribavirin, 1-β-D-ribofuranosyl-1H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. Some embodiments also involve use of derivatives of ribavirin (see, e.g., U.S. Pat. No. 6,277,830). The ribavirin may be administered orally in capsule or tablet form, or in the same or different administration form and in the same or different route as the NS-3 inhibitor compound. Of course, other types of administration of both medicaments, as they become available are contemplated, such as by nasal spray, transdermally, intravenously, by suppository, by sustained release dosage form, etc. Any form of administration will work so long as the proper dosages are delivered without destroying the active ingredient.

In some embodiments, the method further includes administration of ritonavir. Ritonavir, 10-hydroxy-2-methyl-5-(1-methylethyl)-1-[2-(1-methylethyl)-4-thiazolyl]-3,6-dioxo-8,11-bis(phenylmethyl)-2,4,7,12-tetraazamidecan-13-oic acid, 5-thiazolylmethyl ester [5S-(5R*,8R*,10R*,11R*)], available from Abbott Laboratories, is an inhibitor of the protease of the human immunodeficiency virus and also of the cytochrome P450 3A and P450 2D6 liver enzymes frequently involved in hepatic metabolism of therapeutic molecules in man. Because of its strong inhibitory effect on cytochrome P450 3A and the inhibitory effect on cytochrome P450 2D6, ritonavir at doses below the normal therapeutic dosage may be combined with other protease inhibitors to achieve therapeutic levels of the second protease inhibitor while reducing the number of dosage units required, the dosing frequency, or both.

Coadministration of low-dose ritonavir may also be used to compensate for drug interactions that tend to decrease levels of a protease inhibitor metabolized by CYP3A. Its structure, synthesis, manufacture and formulation are described in U.S. Pat. No. 5,541,206 U.S. Pat. No. 5,635,523 U.S. Pat. No. 5,648,497 U.S. Pat. No. 5,846,987 and U.S. Pat. No. 6,232,333. The ritonavir may be administered orally in capsule or tablet or oral solution form, or in the same or different administration form and in the same or different route as the NS-3 inhibitor compound. Of course, other types of administration of both medicaments, as they become available are contemplated, such as by nasal spray, transdermally, intravenously, by suppository, by sustained release dosage form, etc. Any form of administration will work so long as the proper dosages are delivered without destroying the active ingredient.

In some embodiments, an additional antiviral agent is administered during the entire course of NS3 inhibitor compound treatment. In other embodiments, an additional antiviral agent is administered for a period of time that is overlapping with that of the NS3 inhibitor compound treatment, e.g., the additional antiviral agent treatment can begin before the NS3 inhibitor compound treatment begins and end before the NS3 inhibitor compound treatment ends; the additional antiviral agent treatment can begin after the NS3 inhibitor compound treatment begins and end after the NS3 inhibitor compound treatment ends; the additional antiviral agent treatment can begin after the NS3 inhibitor compound treatment begins and end before the NS3 inhibitor compound treatment ends; or the additional antiviral agent treatment can begin before the NS3 inhibitor compound treatment begins and end after the NS3 inhibitor compound treatment ends.

### Methods of Treatment

### Monotherapies

The NS3 inhibitor compounds described herein may be used in acute or chronic therapy for HCV disease. In many embodiments, the NS3 inhibitor compound is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The NS3 inhibitor compound can be administered 5 times per day, 4 times per day, tid, bid, qd, qod, biw, tiw, qw, qow, three times per month, or once monthly. In other embodiments, the NS3 inhibitor compound is administered as a continuous infusion.

In many embodiments, an NS3 inhibitor compound of the embodiments is administered orally.

In connection with the above-described methods for the treatment of HCV disease in a patient, an NS3 inhibitor compound as described herein may be administered to the patient at a dosage from about 0.01 mg to about 100 mg/kg patient bodyweight per day, in 1 to 5 divided doses per day. In some embodiments, the NS3 inhibitor compound is administered at a dosage of about 0.5 mg to about 75 mg/kg patient bodyweight per day, in 1 to 5 divided doses per day.

The amount of active ingredient that may be combined with carrier materials to produce a dosage form can vary depending on the host to be treated and the particular mode of administration. A typical pharmaceutical preparation can contain from about 5% to about 95% active ingredient (w/w). In other embodiments, the pharmaceutical preparation can contain from about 20% to about 80% active ingredient.

Those of skill will readily appreciate that dose levels can vary as a function of the specific NS3 inhibitor compound, the severity of the symptoms and the susceptibility of the subject to side effects. Preferred dosages for a given NS3 inhibitor compound are readily determinable by those of skill in the art by a variety of means. A preferred means is to measure the physiological potency of a given interferon receptor agonist.

In many embodiments, multiple doses of NS3 inhibitor compound are administered. For example, an NS3 inhibitor compound is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Combination Therapies with Ribavirin

In some embodiments, the methods provide for combination therapy comprising administering an NS3 inhibitor compound as described above, and an effective amount of ribavirin. Ribavirin can be administered in dosages of about 400 mg, about 800 mg, about 1000 mg, or about 1200 mg per day.

One embodiment provides any of the above-described methods modified to include co-administering to the patient a therapeutically effective amount of ribavirin for the duration of the desired course of NS3 inhibitor compound treatment.

Another embodiment provides any of the above-described methods modified to include co-administering to the patient about 800 mg to about 1200 mg ribavirin orally per day for the duration of the desired course of NS3 inhibitor compound treatment. In another embodiment, any of the above-described methods may be modified to include co-administering to the patient (a) 1000 mg ribavirin orally per day if the patient has a body weight less than 75 kg or (b) 1200 mg ribavirin orally per day if the patient has a body weight greater than or equal to 75 kg, where the daily dosage of ribavirin is optionally divided into to 2 doses for the duration of the desired course of NS3 inhibitor compound treatment.

Combination Therapies with Levovirin

In some embodiments, the methods provide for combination therapy comprising administering an NS3 inhibitor compound as described above, and an effective amount of levovirin. Levovirin is generally administered in an amount ranging from about 30 mg to about 60 mg, from about 60 mg to about 125 mg, from about 125 mg to about 200 mg, from about 200 mg to about 300 gm, from about 300 mg to about 400 mg, from about 400 mg to about 1200 mg, from about 600 mg to about 1000 mg, or from about 700 to about 900 mg per day, or about 10 mg/kg body weight per day. In some embodiments, levovirin is administered orally in dosages of about 400, about 800, about 1000, or about 1200 mg per day for the desired course of NS3 inhibitor compound treatment.

Combination Therapies with Viramidine

In some embodiments, the methods provide for combination therapy comprising administering an NS3 inhibitor compound as described above, and an effective amount of viramidine. Viramidine is generally administered in an amount ranging from about 30 mg to about 60 mg, from about 60 mg to about 125 mg, from about 125 mg to about 200 mg, from about 200 mg to about 300 mg, from about 300 mg to about 400 mg, from about 400 mg to about 1200 mg, from about 600 mg to about 1000 mg, or from about 700 to about 900 mg per day, or about 10 mg/kg body weight per day. In some embodiments, viramidine is administered orally in dosages of about 800 mg, or about 1600 mg per day for the desired course of NS3 inhibitor compound treatment.

Combination Therapies with Ritonavir

In some embodiments, the methods provide for combination therapy comprising administering an NS3 inhibitor compound as described above, and an effective amount of ritonavir. Ritonavir is generally administered in an amount ranging from about 50 mg to about 100 mg, from about 100 mg to about 200 mg, from about 200 mg to about 300 mg, from about 300 mg to about 400 mg, from about 400 mg to about 500 mg, or from about 500 mg to about 600 mg, twice per day. In some embodiments, ritonavir is administered orally in dosages of about 300 mg, or about 400 mg, or about 600 mg twice per day for the desired course of NS3 inhibitor compound treatment.

Combination Therapies with Alpha-Glucosidase Inhibitors

Suitable α-glucosidase inhibitors include any of the above-described imino-sugars, including long-alkyl chain derivatives of imino sugars as disclosed in U.S. Patent Publication No. 2004/0110795; inhibitors of endoplasmic reticulum-associated α-glucosidases; inhibitors of membrane bound α-glucosidase; miglitol (Glyset®), and active derivatives, and analogs thereof; and acarbose (Precose®), and active derivatives, and analogs thereof.

In many embodiments, the methods provide for combination therapy comprising administering an NS3 inhibitor compound as described above, and an effective amount of an α-glucosidase inhibitor administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time.

An α-glucosidase inhibitor can be administered 5 times per day, 4 times per day, tid (three times daily), bid, qd, qod, biw, tiw, qw, qow, three times per month, or once monthly. In other embodiments, an α-glucosidase inhibitor is administered as a continuous infusion.

In many embodiments, an α-glucosidase inhibitor is administered orally.

In connection with the above-described methods for the treatment of a flavivirus infection, treatment of HCV infection, and treatment of liver fibrosis that occurs as a result of an HCV infection, the methods provide for combination therapy comprising administering an NS3 inhibitor compound as described above, and an effective amount of α-glucosidase inhibitor administered to the patient at a dosage of from about 10 mg per day to about 600 mg per day in divided doses, e.g., from about 10 mg per day to about 30 mg per day, from about 30 mg per day to about 60 mg per day, from about 60 mg per day to about 75 mg per day, from about 75 mg per day to about 90 mg per day, from about 90 mg per day to about 120 mg per day, from about 120 mg per day to about 150 mg per day, from about 150 mg per day to about 180 mg per day, from about 180 mg per day to about 210 mg per day, from about 210 mg per day to about 240 mg per day, from about 240 mg per day to about 270 mg per day, from about 270 mg per day to about 300 mg per day, from about 300 mg per day to about 360 mg per day, from about 360 mg per day to about 420 mg per day, from about 420 mg per day to about 480 mg per day, or from about 480 mg to about 600 mg per day.

In some embodiments, the methods provide for combination therapy comprising administering an NS3 inhibitor compound as described above, and an effective amount of α-glucosidase inhibitor administered in a dosage of about 10 mg three times daily. In some embodiments, an α-glucosidase inhibitor is administered in a dosage of about 15 mg three times daily. In some embodiments, an α-glucosidase inhibitor is administered in a dosage of about 20 mg three times daily. In some embodiments, an α-glucosidase inhibitor is administered in a dosage of about 25 mg three times daily. In some embodiments, an α-glucosidase inhibitor is administered in a dosage of about 30 mg three times daily. In some embodiments, an α-glucosidase inhibitor is administered in a dosage of about 40 mg three times daily. In some embodiments, an α-glucosidase inhibitor is administered in a dosage of about 50 mg three times daily. In some embodiments, an α-glucosidase inhibitor is administered in a dosage of about 100 mg three times daily. In some embodiments, an α-glucosidase inhibitor is administered in a dosage of about 75 mg per day to about 150 mg per day in two or three divided doses, where the individual weighs 60 kg or less. In some embodiments, an α-glucosidase inhibitor is administered in a dosage of about 75 mg per day to about 300 mg per day in two or three divided doses, where the individual weighs 60 kg or more.

The amount of active ingredient (e.g., α-glucosidase inhibitor) that may be combined with carrier materials to produce a dosage form can vary depending on the host to be treated and the particular mode of administration. A typical pharmaceutical preparation can contain from about 5% to about 95% active ingredient (w/w). In other embodiments, the pharmaceutical preparation can contain from about 20% to about 80% active ingredient.

Those of skill will readily appreciate that dose levels can vary as a function of the specific α-glucosidase inhibitor, the severity of the symptoms and the susceptibility of the subject to side effects. Preferred dosages for a given α-glucosidase inhibitor are readily determinable by those of skill in the art by a variety of means. A typical means is to measure the physiological potency of a given active agent.

In many embodiments, multiple doses of an α-glucosidase inhibitor are administered. For example, the methods provide for combination therapy comprising administering an NS3 inhibitor compound as described above, and an effective amount of α-glucosidase inhibitor administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Combination Therapies with Thymosin-α

In some embodiments, the methods provide for combination therapy comprising administering an NS3 inhibitor compound as described above, and an effective amount of thymosin-α. Thymosin-α (Zadaxin™) is generally administered by subcutaneous injection. Thymosin-α can be administered tid, bid, qd, qod, biw, tiw, qw, qow, three times per month, once monthly, substantially continuously, or continuously for the desired course of NS3 inhibitor compound treatment. In many embodiments, thymosin-α is administered twice per week for the desired course of NS3 inhibitor compound treatment. Effective dosages of thymosin-α range from about 0.5 mg to about 5 mg, e.g., from about 0.5 mg to about 1.0 mg, from about 1.0 mg to about 1.5 mg, from about 1.5 mg to about 2.0 mg, from about 2.0 mg to about 2.5 mg, from about 2.5 mg to about 3.0 mg, from about 3.0 mg to about 3.5 mg, from about 3.5 mg to about 4.0 mg, from about 4.0 mg to about 4.5 mg, or from about 4.5 mg to about 5.0 mg. In particular embodiments, thymosin-α is administered in dosages containing an amount of 1.0 mg or 1.6 mg.

Thymosin-α can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. In one embodiment, thymosin-α is administered for the desired course of NS3 inhibitor compound treatment.

Combination Therapies with Interferon(s)

In many embodiments, the methods provide for combination therapy comprising administering an NS3 inhibitor compound as described above, and an effective amount of an interferon receptor agonist. In some embodiments, a compound of Formulae I, Ia, II, III, IV, V, VI-1, VI-2, VII, VIII, IX, X, XI, or, XII, or any compounds disclosed herein and a Type I or III interferon receptor agonist are co-administered in the treatment methods described herein. Type I interferon receptor agonists suitable for use herein include any interferon-α (IFN-α). In certain embodiments, the interferon-α is a PEGylated interferon-α. In certain other embodiments, the interferon-α is a consensus interferon, such as INFERGEN® interferon alfacon-1. In still other embodiments, the interferon-α is a monoPEG (30 kD, linear)-ylated consensus interferon.

Effective dosages of an IFN-α range from about 3 μg to about 27 μg, from about 3 MU to about 10 MU, from about 90 μg to about 180 μg, or from about 18 μg to about 90 μg. Effective dosages of Infergen® consensus IFN-α include about 3 μg, about 6 μg, about 9 μg, about 12 μg, about 15 μg, about 18 μg, about 21 μg, about 24 μg, about 27 μg, or about 30 μg, of drug per dose. Effective dosages of IFN-α2a and IFN-α2b range from 3 million Units (MU) to 10 MU per dose. Effective dosages of PEGASYS®PEGylated IFN-α2a contain an amount of about 90 μg to 270 μg, or about 180 μg, of drug per dose. Effective dosages of PEG-INTRON®PEGylated IFN-α2b contain an amount of about 0.5 to 3.0 μg of drug per kg of body weight per dose. Effective dosages of PEGylated consensus interferon (PEG-CIFN) contain an amount of about 18 μg to about 90 μg, or from about 27 μg to about 60 μg, or about 45 μg, of CIFN amino acid weight per dose of PEG-CIFN. Effective dosages of monoPEG (30 kD, linear)-ylated CIFN contain an amount of about 45 μg to about 270 μg, or about 60 μg to about 180 μg, or about 90 μg to about 120 μg, of drug per dose. IFN-α can be administered daily, every other day, once a week, three times a week, every other week, three times per month, once monthly, substantially continuously or continuously.

In many embodiments, the Type I or Type III interferon receptor agonist and/or the Type II interferon receptor agonist is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. Dosage regimens can include tid, bid, qd, qod, biw, tiw, qw, qow, three times per month, or monthly administrations. Some embodiments provide any of the above-described methods in which the desired dosage of IFN-α is administered subcutaneously to the patient by bolus delivery qd, qod, tiw, biw, qw, qow, three times per month, or monthly, or is administered subcutaneously to the patient per day by substantially continuous or continuous delivery, for the desired treatment duration. In other embodiments, any of the above-described methods may be practiced in which the desired dosage of PEGylated IFN-α (PEG-IFN-α) is administered subcutaneously to the patient by bolus delivery qw, qow, three times per month, or monthly for the desired treatment duration.

In other embodiments, an NS3 inhibitor compound and a Type II interferon receptor agonist are co-administered in the treatment methods of the embodiments. Type II interferon receptor agonists suitable for use herein include any interferon-γ (IFN-γ).

Effective dosages of IFN-γ can range from about 0.5 μg/m² to about 500 μg/m², usually from about 1.5 μg/m² to 200 μg/m², depending on the size of the patient. This activity is based on 10⁶ international units (U) per 50 μg of protein. IFN-γ can be administered daily, every other day, three times a week, or substantially continuously or continuously.

In specific embodiments of interest, IFN-γ is administered to an individual in a unit dosage form of from about 25 μg to about 500 μg, from about 50 μg to about 400 μg, or from about 100 μg to about 300 μg. In particular embodiments of interest, the dose is about 200 μg IFN-γ. In many embodiments of interest, IFN-γ1b is administered.

Where the dosage is 200 μg IFN-γ per dose, the amount of IFN-γ per body weight (assuming a range of body weights of from about 45 kg to about 135 kg) is in the range of from about 4.4 μg IFN-γ per kg body weight to about 1.48 μg IFN-γ per kg body weight.

The body surface area of subject individuals generally ranges from about 1.33 m² to about 2.50 m². Thus, in many embodiments, an IFN-γ dosage ranges from about 150 μg/m² to about 20 μg/m². For example, an IFN-γ dosage ranges from about 20 μg/m² to about 30 μg/m², from about 30 μg/m² to about 40 μg/m², from about 40 μg/m² to about 50 μg/m², from about 50 μg/m² to about 60 μg/m², from about 60 μg/m² to about 70 μg/m², from about 70 μg/m² to about 80 μg/m², from about 80 μg/m² to about 90 μg/m², from about 90 μg/m² to about 100 μg/m², from about 100 μg/m² to about 110 μg/m², from about 110 μg/m² to about 120 μg/m², from about 120 μg/m² to about 130 μg/m², from about 130 μg/m² to about 140 μg/m², or from about 140 μg/m² to about 150 μg/m². In some embodiments, the dosage groups range from about 25 μg/m² to about 100 μg/m². In other embodiments, the dosage groups range from about 25 μg/m² to about 50 μg/m².

In some embodiments, a Type I or a Type III interferon receptor agonist is administered in a first dosing regimen, followed by a second dosing regimen. The first dosing regimen of Type I or a Type III interferon receptor agonist (also referred to as “the induction regimen”) generally involves administration of a higher dosage of the Type I or Type III interferon receptor agonist. For example, in the case of Infergen® consensus IFN-α (CIFN), the first dosing regimen comprises administering CIFN at about 9 μg, about 15 μg, about 18 μg, or about 27 μg. The first dosing regimen can encompass a single dosing event, or at least two or more dosing events. The first dosing regimen of the Type I or Type III interferon receptor agonist can be administered daily, every other day, three times a week, every other week, three times per month, once monthly, substantially continuously or continuously.

The first dosing regimen of the Type I or Type III interferon receptor agonist is administered for a first period of time, which time period can be at least about 4 weeks, at least about 8 weeks, or at least about 12 weeks.

The second dosing regimen of the Type I or Type III interferon receptor agonist (also referred to as “the maintenance dose”) generally involves administration of a lower amount of the Type I or Type III interferon receptor agonist. For example, in the case of CIFN, the second dosing regimen comprises administering CIFN at a dose of at least about 3 μg, at least about 9 μg, at least about 15 μg, or at least about 18 μg. The second dosing regimen can encompass a single dosing event, or at least two or more dosing events.

The second dosing regimen of the Type I or Type III interferon receptor agonist can be administered daily, every other day, three times a week, every other week, three times per month, once monthly, substantially continuously or continuously.

In some embodiments, where an “induction”/“maintenance” dosing regimen of a Type I or a Type III interferon receptor agonist is administered, a “priming” dose of a Type II interferon receptor agonist (e.g., IFN-γ) is included. In these embodiments, IFN-γ is administered for a period of time from about 1 day to about 14 days, from about 2 days to about 10 days, or from about 3 days to about 7 days, before the beginning of treatment with the Type I or Type III interferon receptor agonist. This period of time is referred to as the “priming” phase.

In some of these embodiments, the Type II interferon receptor agonist treatment is continued throughout the entire period of treatment with the Type I or Type III interferon receptor agonist. In other embodiments, the Type II interferon receptor agonist treatment is discontinued before the end of treatment with the Type I or Type III interferon receptor agonist. In these embodiments, the total time of treatment with Type II interferon receptor agonist (including the “priming” phase) is from about 2 days to about 30 days, from about 4 days to about 25 days, from about 8 days to about 20 days, from about 10 days to about 18 days, or from about 12 days to about 16 days. In still other embodiments, the Type II interferon receptor agonist treatment is discontinued once Type I or a Type III interferon receptor agonist treatment begins.

In other embodiments, the Type I or Type III interferon receptor agonist is administered in single dosing regimen. For example, in the case of CIFN, the dose of CIFN is generally in a range of from about 3 μg to about 15 μg, or from about 9 μg to about 15 μg. The dose of Type I or a Type III interferon receptor agonist is generally administered daily, every other day, three times a week, every other week, three times per month, once monthly, or substantially continuously. The dose of the Type I or Type III interferon receptor agonist is administered for a period of time, which period can be, for example, from at least about 24 weeks to at least about 48 weeks, or longer.

In some embodiments, where a single dosing regimen of a Type I or a Type III interferon receptor agonist is administered, a “priming” dose of a Type II interferon receptor agonist (e.g., IFN-γ) is included. In these embodiments, IFN-γ is administered for a period of time from about 1 day to about 14 days, from about 2 days to about 10 days, or from about 3 days to about 7 days, before the beginning of treatment with the Type I or Type III interferon receptor agonist. This period of time is referred to as the “priming” phase. In some of these embodiments, the Type II interferon receptor agonist treatment is continued throughout the entire period of treatment with the Type I or Type III interferon receptor agonist. In other embodiments, the Type II interferon receptor agonist treatment is discontinued before the end of treatment with the Type I or Type III interferon receptor agonist. In these embodiments, the total time of treatment with the Type II interferon receptor agonist (including the “priming” phase) is from about 2 days to about 30 days, from about 4 days to about 25 days, from about 8 days to about 20 days, from about 10 days to about 18 days, or from about 12 days to about 16 days. In still other embodiments, Type II interferon receptor agonist treatment is discontinued once Type I or a Type III interferon receptor agonist treatment begins.

In additional embodiments, an NS3 inhibitor compound, a Type I or III interferon receptor agonist, and a Type II interferon receptor agonist are co-administered for the desired duration of treatment in the methods described herein. In some embodiments, an NS3 inhibitor compound, an interferon-α, and an interferon-γ are co-administered for the desired duration of treatment in the methods described herein.

In some embodiments, the invention provides methods using an amount of a Type I or Type III interferon receptor agonist, a Type II interferon receptor agonist, and an NS3 inhibitor compound, effective for the treatment of HCV infection in a patient. Some embodiments provide methods using an effective amount of an IFN-α, IFN-γ, and an NS3 inhibitor compound in the treatment of HCV infection in a patient. One embodiment provides a method using an effective amount of a consensus IFN-α, IFN-γ and an NS3 inhibitor compound in the treatment of HCV infection in a patient.

In general, an effective amount of a consensus interferon (CIFN) and IFN-γ suitable for use in the methods of the embodiments is provided by a dosage ratio of 1 μg CIFN:10 μg IFN-γ, where both CIFN and IFN-γ are unPEGylated and unglycosylated species.

In one embodiment, the invention provides any of the above-described methods modified to use an effective amount of INFERGEN®consensus IFN-α and IFN-γ in the treatment of HCV infection in a patient comprising administering to the patient a dosage of INFERGEN® containing an amount of about 1 μg to about 30 μg, of drug per dose of INFERGEN®, subcutaneously qd, qod, tiw, biw, qw, qow, three times per month, once monthly, or per day substantially continuously or continuously, in combination with a dosage of IFN-γ containing an amount of about 10 μg to about 300 μg of drug per dose of IFN-γ, subcutaneously qd, qod, tiw, biw, qw, qow, three times per month, once monthly, or per day substantially continuously or continuously, for the desired duration of treatment with an NS3 inhibitor compound.

Another embodiment provides any of the above-described methods modified to use an effective amount of INFERGEN®consensus IFN-α and IFN-γ in the treatment of virus infection in a patient comprising administering to the patient a dosage of INFERGEN® containing an amount of about 1 μg to about 9 μg, of drug per dose of INFERGEN®, subcutaneously qd, qod, tiw, biw, qw, qow, three times per month, once monthly, or per day substantially continuously or continuously, in combination with a dosage of IFN-γ containing an amount of about 10 μg to about 100 μg of drug per dose of IFN-γ, subcutaneously qd, qod, tiw, biw, qw, qow, three times per month, once monthly, or per day substantially continuously or continuously, for the desired duration of treatment with an NS3 inhibitor compound.

Another embodiment provides any of the above-described methods modified to use an effective amount of INFERGEN®consensus IFN-α and IFN-γ in the treatment of virus infection in a patient comprising administering to the patient a dosage of INFERGEN® containing an amount of about 1 μg of drug per dose of INFERGEN®, subcutaneously qd, qod, tiw, biw, qw, qow, three times per month, once monthly, or per day substantially continuously or continuously, in combination with a dosage of IFN-γ containing an amount of about 10 μg to about 50 μg of drug per dose of IFN-γ, subcutaneously qd, qod, tiw, biw, qw, qow, three times per month, once monthly, or per day substantially continuously or continuously, for the desired duration of treatment with an NS3 inhibitor compound.

Another embodiment provides any of the above-described methods modified to use an effective amount of INFERGEN®consensus IFN-α and IFN-γ in the treatment of a virus infection in a patient comprising administering to the patient a dosage of INFERGEN® containing an amount of about 9 μg of drug per dose of INFERGEN®, subcutaneously qd, qod, tiw, biw, qw, qow, three times per month, once monthly, or per day substantially continuously or continuously, in combination with a dosage of IFN-γ containing an amount of about 90 μg to about 100 μg of drug per dose of IFN-γ, subcutaneously qd, qod, tiw, biw, qw, qow, three times per month, once monthly, or per day substantially continuously or continuously, for the desired duration of treatment with an NS3 inhibitor compound.

Another embodiment provides any of the above-described methods modified to use an effective amount of INFERGEN®consensus IFN-α and IFN-γ in the treatment of a virus infection in a patient comprising administering to the patient a dosage of INFERGEN® containing an amount of about 30 μg of drug per dose of INFERGEN®, subcutaneously qd, qod, tiw, biw, qw, qow, three times per month, once monthly, or per day substantially continuously or continuously, in combination with a dosage of IFN-γ containing an amount of about 200 μg to about 300 μg of drug per dose of IFN-γ, subcutaneously qd, qod, tiw, biw, qw, qow, three times per month, once monthly, or per day substantially continuously or continuously, for the desired duration of treatment with an NS3 inhibitor compound.

Another embodiment provides any of the above-described methods modified to use an effective amount of PEGylated consensus IFN-α and IFN-γ in the treatment of a virus infection in a patient comprising administering to the patient a dosage of PEGylated consensus IFN-α (PEG-CIFN) containing an amount of about 4 μg to about 60 μg of CIFN amino acid weight per dose of PEG-CIFN, subcutaneously qw, qow, three times per month, or monthly, in combination with a total weekly dosage of IFN-γ containing an amount of about 30 μg to about 1,000 μg of drug per week in divided doses administered subcutaneously qd, qod, tiw, biw, or administered substantially continuously or continuously, for the desired duration of treatment with an NS3 inhibitor compound.

Another embodiment provides any of the above-described methods modified to use an effective amount of PEGylated consensus IFN-α and IFN-γ in the treatment of a virus infection in a patient comprising administering to the patient a dosage of PEGylated consensus IFN-α (PEG-CIFN) containing an amount of about 18 μg to about 24 μg of CIFN amino acid weight per dose of PEG-CIFN, subcutaneously qw, qow, three times per month, or monthly, in combination with a total weekly dosage of IFN-γ containing an amount of about 100 μg to about 300 μg of drug per week in divided doses administered subcutaneously qd, qod, tiw, biw, or substantially continuously or continuously, for the desired duration of treatment with an NS3 inhibitor compound.

In general, an effective amount of IFN-α 2a or 2b or 2c and IFN-γ suitable for use in the methods of the embodiments is provided by a dosage ratio of 1 million Units (MU) IFN-α 2a or 2b or 2c: 30 μg IFN-γ, where both IFN-α 2a or 2b or 2c and IFN-γ are unPEGylated and unglycosylated species.

Another embodiment provides any of the above-described methods modified to use an effective amount of IFN-α 2a or 2b or 2c and IFN-γ in the treatment of a virus infection in a patient comprising administering to the patient a dosage of IFN-α 2a, 2b or 2c containing an amount of about 1 MU to about 20 MU of drug per dose of IFN-α 2a, 2b or 2c subcutaneously qd, qod, tiw, biw, or per day substantially continuously or continuously, in combination with a dosage of IFN-γ containing an amount of about 30 μg to about 600 μg of drug per dose of IFN-γ, subcutaneously qd, qod, tiw, biw, or per day substantially continuously or continuously, for the desired duration of treatment with an NS3 inhibitor compound.

Another embodiment provides any of the above-described methods modified to use an effective amount of IFN-α 2a or 2b or 2c and IFN-γ in the treatment of a virus infection in a patient comprising administering to the patient a dosage of IFN-α 2a, 2b or 2c containing an amount of about 3 MU of drug per dose of IFN-α 2a, 2b or 2c subcutaneously qd, qod, tiw, biw, or per day substantially continuously or continuously, in combination with a dosage of IFN-γ containing an amount of about 100 μg of drug per dose of IFN-γ, subcutaneously qd, qod, tiw, biw, or per day substantially continuously or continuously, for the desired duration of treatment with an NS3 inhibitor compound.

Another embodiment provides any of the above-described methods modified to use an effective amount of IFN-α 2a or 2b or 2c and IFN-γ in the treatment of a virus infection in a patient comprising administering to the patient a dosage of IFN-α 2a, 2b or 2c containing an amount of about 10 MU of drug per dose of IFN-α 2a, 2b or 2c subcutaneously qd, qod, tiw, biw, or per day substantially continuously or continuously, in combination with a dosage of IFN-γ containing an amount of about 300 μg of drug per dose of IFN-γ, subcutaneously qd, qod, tiw, biw, or per day substantially continuously or continuously, for the desired duration of treatment with an NS3 inhibitor compound.

Another embodiment provides any of the above-described methods modified to use an effective amount of PEGASYS®PEGylated IFN-α2a and IFN-γ in the treatment of a virus infection in a patient comprising administering to the patient a dosage of PEGASYS® containing an amount of about 90 μg to about 360 μg, of drug per dose of PEGASYS®, subcutaneously qw, qow, three times per month, or monthly, in combination with a total weekly dosage of IFN-γ containing an amount of about 30 μg to about 1,000 μg, of drug per week administered in divided doses subcutaneously qd, qod, tiw, or biw, or administered substantially continuously or continuously, for the desired duration of treatment with an NS3 inhibitor compound.

Another embodiment provides any of the above-described methods modified to use an effective amount of PEGASYS®PEGylated IFN-α2a and IFN-γ in the treatment of a virus infection in a patient comprising administering to the patient a dosage of PEGASYS® containing an amount of about 180 μg of drug per dose of PEGASYS®, subcutaneously qw, qow, three times per month, or monthly, in combination with a total weekly dosage of IFN-γ containing an amount of about 100 μg to about 300 μg, of drug per week administered in divided doses subcutaneously qd, qod, tiw, or biw, or administered substantially continuously or continuously, for the desired duration of treatment with an NS3 inhibitor compound.

Another embodiment provides any of the above-described methods modified to use an effective amount of PEG-INTRON®PEGylated IFN-α2b and IFN-γ in the treatment of a virus infection in a patient comprising administering to the patient a dosage of PEG-INTRON® containing an amount of about 0.75 μg to about 3.0 μg of drug per kilogram of body weight per dose of PEG-INTRON®, subcutaneously qw, qow, three times per month, or monthly, in combination with a total weekly dosage of IFN-γ containing an amount of about 30 μg to about 1,000 μg of drug per week administered in divided doses subcutaneously qd, qod, tiw, or biw, or administered substantially continuously or continuously, for the desired duration of treatment with an NS3 inhibitor compound.

Another embodiment provides any of the above-described methods modified to use an effective amount of PEG-INTRON®PEGylated IFN-α2b and IFN-γ in the treatment of a virus infection in a patient comprising administering to the patient a dosage of PEG-INTRON® containing an amount of about 1.5 μg of drug per kilogram of body weight per dose of PEG-INTRON®, subcutaneously qw, qow, three times per month, or monthly, in combination with a total weekly dosage of IFN-γ containing an amount of about 100 μg to about 300 μg of drug per week administered in divided doses subcutaneously qd, qod, tiw, or biw, or administered substantially continuously or continuously, for the desired duration of treatment with an NS3 inhibitor compound.

One embodiment provides any of the above-described methods modified to comprise administering to an individual having an HCV infection an effective amount of an NS3 inhibitor; and a regimen of 9 μg INFERGEN® consensus IFN-α administered subcutaneously qd or tiw, and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 9 μg INFERGEN® consensus IFN-α administered subcutaneously qd or tiw; 50 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 9 μg INFERGEN® consensus IFN-α administered subcutaneously qd or tiw; 100 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 9 μg INFERGEN® consensus IFN-α administered subcutaneously qd or tiw; and 50 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS3 inhibitor; and a regimen of 9 μg INFERGEN® consensus IFN-α administered subcutaneously qd or tiw; and 100 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS3 inhibitor; and a regimen of 9 μg INFERGEN® consensus IFN-α administered subcutaneously qd or tiw; 25 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 9 μg INFERGEN® consensus IFN-α administered subcutaneously qd or tiw; 200 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 9 μg INFERGEN® consensus IFN-α administered subcutaneously qd or tiw; and 25 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS3 inhibitor; and a regimen of 9 μg INFERGEN® consensus IFN-α administered subcutaneously qd or tiw; and 200 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS3 inhibitor; and a regimen of 100 μg monoPEG(30 kD, linear)-ylated consensus IFN-α administered subcutaneously every 10 days or qw, and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 100 μg monoPEG(30 kD, linear)-ylated consensus IFN-α administered subcutaneously every 10 days or qw; 50 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 100 μg monoPEG(30 kD, linear)-ylated consensus IFN-α administered subcutaneously every 10 days or qw; 100 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 100 μg monoPEG(30 kD, linear)-ylated consensus IFN-α administered subcutaneously every 10 days or qw; and 50 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS3 inhibitor; and a regimen of 100 μg monoPEG(30 kD, linear)-ylated consensus IFN-α administered subcutaneously every 10 days or qw; and 100 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS3 inhibitor; and a regimen of 150 μg monoPEG(30 kD, linear)-ylated consensus IFN-α administered subcutaneously every 10 days or qw, and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 150 μg monoPEG(30 kD, linear)-ylated consensus IFN-α administered subcutaneously every 10 days or qw; 50 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 150 μg monoPEG(30 kD, linear)-ylated consensus IFN-α administered subcutaneously every 10 days or qw; 100 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 150 μg monoPEG(30 kD, linear)-ylated consensus IFN-α administered subcutaneously every 10 days or qw; and 50 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS3 inhibitor; and a regimen of 150 μg monoPEG(30 kD, linear)-ylated consensus IFN-α administered subcutaneously every 10 days or qw; and 100 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS3 inhibitor; and a regimen of 200 μg monoPEG(30 kD, linear)-ylated consensus IFN-α administered subcutaneously every 10 days or qw, and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 200 μg monoPEG(30 kD, linear)-ylated consensus IFN-α administered subcutaneously every 10 days or qw; 50 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 200 μg monoPEG(30 kD, linear)-ylated consensus IFN-α administered subcutaneously every 10 days or qw; 100 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 200 μg monoPEG(30 kD, linear)-ylated consensus IFN-α administered subcutaneously every 10 days or qw; and 50 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS3 inhibitor; and a regimen of 200 μg monoPEG(30 kD, linear)-ylated consensus IFN-α administered subcutaneously every 10 days or qw; and 100 μg Actimmune® human IFN-γ1b administered subcutaneously tiw, where the duration of therapy is 48 weeks.

Any of the above-described methods involving administering an NS3 inhibitor, a Type I interferon receptor agonist (e.g., an IFN-α), and a Type II interferon receptor agonist (e.g., an IFN-γ), can be augmented by administration of an effective amount of a TNF-α antagonist (e.g., a TNF-α antagonist other than pirfenidone or a pirfenidone analog). Exemplary, non-limiting TNF-α antagonists that are suitable for use in such combination therapies include ENBREL®, REMICADE®, and HUMIRA™.

One embodiment provides a method using an effective amount of ENBREL®; an effective amount of IFN-α; an effective amount of IFN-γ; and an effective amount of an NS3 inhibitor in the treatment of an HCV infection in a patient, comprising administering to the patient a dosage ENBREL® containing an amount of from about 0.1 to about 23 mg per dose, from about 0.1 μg to about 1 μg, from about 1 μg to about 10 μg, from about 10 μg to about 100 μg, from about 100 μg to about 1 mg, from about 1 mg to about 5 mg, from about 5 mg to about 10 mg, from about 10 mg to about 15 mg, from about 15 mg to about 20 mg, or from about 20 mg to about 23 mg of ENBREL®, subcutaneously qd, qod, tiw, biw, qw, qow, three times per month, once monthly, or once every other month, or per day substantially continuously or continuously, for the desired duration of treatment.

One embodiment provides a method using an effective amount of REMICADE®, an effective amount of IFN-α; an effective amount of IFN-γ; and an effective amount of an NS3 inhibitor in the treatment of an HCV infection in a patient, comprising administering to the patient a dosage of REMICADE® containing an amount of from about 0.1 mg/kg to about 4.5 mg/kg, from about 0.1 mg/kg to about 0.5 mg/kg, from about 0.5 mg/kg to about 1.0 mg/kg, from about 1.0 mg/kg to about 1.5 mg/kg, from about 1.5 mg/kg to about 2.0 mg/kg, from about 2.0 mg/kg to about 2.5 mg/kg, from about 2.5 mg/kg to about 3.0 mg/kg, from about 3.0 mg/kg to about 3.5 mg/kg, from about 3.5 mg/kg to about 4.0 mg/kg, or from about 4.0 mg/kg to about 4.5 mg/kg per dose of REMICADE®, intravenously qd, qod, tiw, biw, qw, qow, three times per month, once monthly, or once every other month, or per day substantially continuously or continuously, for the desired duration of treatment.

One embodiment provides a method using an effective amount of HUMIRA™, an effective amount of IFN-α; an effective amount of IFN-γ; and an effective amount of an NS3 inhibitor in the treatment of an HCV infection in a patient, comprising administering to the patient a dosage of HUMIRA™ containing an amount of from about 0.1 to about 35 mg, from about 0.1 μg to about 1 μg, from about 1 μg to about 10 μg, from about 10 μg to about 100 μg, from about 100 μg to about 1 mg, from about 1 mg to about 5 mg, from about 5 mg to about 10 mg, from about 10 mg to about 15 mg, from about 15 mg to about 20 mg, from about 20 mg to about 25 mg, from about 25 mg to about 30 mg, or from about 30 mg to about 35 mg per dose of a HUMIRA™, subcutaneously qd, qod, tiw, biw, qw, qow, three times per month, once monthly, or once every other month, or per day substantially continuously or continuously, for the desired duration of treatment.

Combination Therapies with Pirfenidone

In many embodiments, the methods provide for combination therapy comprising administering an NS3 inhibitor compound as described above, and an effective amount of pirfenidone or a pirfenidone analog. In some embodiments, an NS3 inhibitor compound, one or more interferon receptor agonist(s), and pirfenidone or pirfenidone analog are co-administered in the treatment methods of the embodiments. In certain embodiments, an NS3 inhibitor compound, a Type I interferon receptor agonist, and pirfenidone (or a pirfenidone analog) are co-administered. In other embodiments, an NS3 inhibitor compound, a Type I interferon receptor agonist, a Type II interferon receptor agonist, and pirfenidone (or a pirfenidone analog) are co-administered. Type I interferon receptor agonists suitable for use herein include any IFN-α, such as interferon alfa-2a, interferon alfa-2b, interferon alfacon-1, and PEGylated IFN-α's, such as peginterferon alfa-2a, peginterferon alfa-2b, and PEGylated consensus interferons, such as monoPEG (30 kD, linear)-ylated consensus interferon. Type II interferon receptor agonists suitable for use herein include any interferon-γ.

Pirfenidone or a pirfenidone analog can be administered once per month, twice per month, three times per month, once per week, twice per week, three times per week, four times per week, five times per week, six times per week, daily, or in divided daily doses ranging from once daily to 5 times daily over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Effective dosages of pirfenidone or a specific pirfenidone analog include a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally in one to five divided doses per day. Other doses and formulations of pirfenidone and specific pirfenidone analogs suitable for use in the treatment of fibrotic diseases are described in U.S. Pat. Nos., 5,310,562; 5,518,729; 5,716,632; and 6,090,822.

One embodiment provides any of the above-described methods modified to include co-administering to the patient a therapeutically effective amount of pirfenidone or a pirfenidone analog for the duration of the desired course of NS3 inhibitor compound treatment.

Combination Therapies with TNF-α Antagonists

In many embodiments, the methods provide for combination therapy comprising administering an effective amount of an NS3 inhibitor compound as described above, and an effective amount of TNF-α antagonist, in combination therapy for treatment of an HCV infection.

Effective dosages of a TNF-α antagonist range from 0.1 μg to 40 mg per dose, e.g., from about 0.1 μg to about 0.5 μg per dose, from about 0.5 μg to about 1.0 μg per dose, from about 1.0 μg per dose to about 5.0 μg per dose, from about 5.0 μg to about 10 μg per dose, from about 10 μg to about 20 μg per dose, from about 20 μg per dose to about 30 μg per dose, from about 30 μg per dose to about 40 μg per dose, from about 40 μg per dose to about 50 μg per dose, from about 50 μg per dose to about 60 μg per dose, from about 60 μg per dose to about 70 μg per dose, from about 70 μg to about 80 μg per dose, from about 80 μg per dose to about 100 μg per dose, from about 100 μg to about 150 μg per dose, from about 150 μg to about 200 μg per dose, from about 200 μg per dose to about 250 μg per dose, from about 250 μg to about 300 μg per dose, from about 300 μg to about 400 μg per dose, from about 400 μg to about 500 μg per dose, from about 500 μg to about 600 μg per dose, from about 600 μg to about 700 μg per dose, from about 700 μg to about 800 μg per dose, from about 800 μg to about 900 μg per dose, from about 900 μg to about 1000 μg per dose, from about 1 mg to about 10 mg per dose, from about 10 mg to about 15 mg per dose, from about 15 mg to about 20 mg per dose, from about 20 mg to about 25 mg per dose, from about 25 mg to about 30 mg per dose, from about 30 mg to about 35 mg per dose, or from about 35 mg to about 40 mg per dose.

In some embodiments, effective dosages of a TNF-α antagonist are expressed as mg/kg body weight. In these embodiments, effective dosages of a TNF-α antagonist are from about 0.1 mg/kg body weight to about 10 mg/kg body weight, e.g., from about 0.1 mg/kg body weight to about 0.5 mg/kg body weight, from about 0.5 mg/kg body weight to about 1.0 mg/kg body weight, from about 1.0 mg/kg body weight to about 2.5 mg/kg body weight, from about 2.5 mg/kg body weight to about 5.0 mg/kg body weight, from about 5.0 mg/kg body weight to about 7.5 mg/kg body weight, or from about 7.5 mg/kg body weight to about 10 mg/kg body weight.

In many embodiments, a TNF-α antagonist is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The TNF-α antagonist can be administered tid, bid, qd, qod, biw, tiw, qw, qow, three times per month, once monthly, substantially continuously, or continuously.

In many embodiments, multiple doses of a TNF-α antagonist are administered. For example, a TNF-α antagonist is administered once per month, twice per month, three times per month, every other week (qow), once per week (qw), twice per week (biw), three times per week (tiw), four times per week, five times per week, six times per week, every other day (qod), daily (qd), twice a day (bid), or three times a day (tid), substantially continuously, or continuously, over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

A TNF-α antagonist and an NS3 inhibitor are generally administered in separate formulations. A TNF-α antagonist and an NS3 inhibitor may be administered substantially simultaneously, or within about 30 minutes, about 1 hour, about 2 hours, about 4 hours, about 8 hours, about 16 hours, about 24 hours, about 36 hours, about 72 hours, about 4 days, about 7 days, or about 2 weeks of one another.

One embodiment provides a method using an effective amount of a TNF-α antagonist and an effective amount of an NS3 inhibitor in the treatment of an HCV infection in a patient, comprising administering to the patient a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

One embodiment provides a method using an effective amount of ENBREL® and an effective amount of an NS3 inhibitor in the treatment of an HCV infection in a patient, comprising administering to the patient a dosage ENBREL® containing an amount of from about 0.1 μg to about 23 mg per dose, from about 0.1 μg to about 1 μg, from about 1 μg to about 10 μg, from about 10 μg to about 100 μg, from about 100 μg to about 1 mg, from about 1 mg to about 5 mg, from about 5 mg to about 10 mg, from about 10 mg to about 15 mg, from about 15 mg to about 20 mg, or from about 20 mg to about 23 mg of ENBREL®, subcutaneously qd, qod, tiw, biw, qw, qow, three times per month, once monthly, or once every other month, or per day substantially continuously or continuously, for the desired duration of treatment with an NS3 inhibitor compound.

One embodiment provides a method using an effective amount of REMICADE® and an effective amount of an NS3 inhibitor in the treatment of an HCV infection in a patient, comprising administering to the patient a dosage of REMICADE® containing an amount of from about 0.1 mg/kg to about 4.5 mg/kg, from about 0.1 mg/kg to about 0.5 mg/kg, from about 0.5 mg/kg to about 1.0 mg/kg, from about 1.0 mg/kg to about 1.5 mg/kg, from about 1.5 mg/kg to about 2.0 mg/kg, from about 2.0 mg/kg to about 2.5 mg/kg, from about 2.5 mg/kg to about 3.0 mg/kg, from about 3.0 mg/kg to about 3.5 mg/kg, from about 3.5 mg/kg to about 4.0 mg/kg, or from about 4.0 mg/kg to about 4.5 mg/kg per dose of REMICADE®, intravenously qd, qod, tiw, biw, qw, qow, three times per month, once monthly, or once every other month, or per day substantially continuously or continuously, for the desired duration of treatment with an NS3 inhibitor compound.

One embodiment provides a method using an effective amount of HUMIRA™ and an effective amount of an NS3 inhibitor in the treatment of an HCV infection in a patient, comprising administering to the patient a dosage of HUMIRA™ containing an amount of from about 0.1 μg to about 35 mg, from about 0.1 μg to about 1 μg, from about 1 μg to about 10 μg, from about 10 μg to about 100 μg, from about 100 μg to about 1 mg, from about 1 mg to about 5 mg, from about 5 mg to about 10 mg, from about 10 mg to about 15 mg, from about 15 mg to about 20 mg, from about 20 mg to about 25 mg, from about 25 mg to about 30 mg, or from about 30 mg to about 35 mg per dose of a HUMIRA™, subcutaneously qd, qod, tiw, biw, qw, qow, three times per month, once monthly, or once every other month, or per day substantially continuously or continuously, for the desired duration of treatment with an NS3 inhibitor compound.

Combination Therapies with Thymosin-α

In many embodiments, the methods provide for combination therapy comprising administering an effective amount of an NS3 inhibitor compound as described above, and an effective amount of thymosin-α, in combination therapy for treatment of an HCV infection.

Effective dosages of thymosin-α range from about 0.5 mg to about 5 mg, e.g., from about 0.5 mg to about 1.0 mg, from about 1.0 mg to about 1.5 mg, from about 1.5 mg to about 2.0 mg, from about 2.0 mg to about 2.5 mg, from about 2.5 mg to about 3.0 mg, from about 3.0 mg to about 3.5 mg, from about 3.5 mg to about 4.0 mg, from about 4.0 mg to about 4.5 mg, or from about 4.5 mg to about 5.0 mg. In particular embodiments, thymosin-α is administered in dosages containing an amount of 1.0 mg or 1.6 mg.

One embodiment provides a method using an effective amount of ZADAXIN™ thymosin-α and an effective amount of an NS3 inhibitor in the treatment of an HCV infection in a patient, comprising administering to the patient a dosage of ZADAXIN™ containing an amount of from about 1.0 mg to about 1.6 mg per dose, subcutaneously twice per week for the desired duration of treatment with the NS3 inhibitor compound.

Combination Therapies with a TNF-α Antagonist and an Interferon

Some embodiments provide a method of treating an HCV infection in an individual having an HCV infection, the method comprising administering an effective amount of an NS3 inhibitor, and effective amount of a TNF-α antagonist, and an effective amount of one or more interferons.

One embodiment provides any of the above-described methods modified to use an effective amount of IFN-γ and an effective amount of a TNF-α antagonist in the treatment of HCV infection in a patient comprising administering to the patient a dosage of IFN-γ containing an amount of about 10 μg to about 300 μg of drug per dose of IFN-γ, subcutaneously qd, qod, tiw, biw, qw, qow, three times per month, once monthly, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

One embodiment provides any of the above-described methods modified to use an effective amount of IFN-γ and an effective amount of a TNF-α antagonist in the treatment of HCV infection in a patient comprising administering to the patient a dosage of IFN-γ containing an amount of about 10 μg to about 100 μg of drug per dose of IFN-γ, subcutaneously qd, qod, tiw, biw, qw, qow, three times per month, once monthly, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of IFN-γ and an effective amount of a TNF-α antagonist in the treatment of a virus infection in a patient comprising administering to the patient a total weekly dosage of IFN-γ containing an amount of about 30 μg to about 1,000 μg of drug per week in divided doses administered subcutaneously qd, qod, tiw, biw, or administered substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of IFN-γ and an effective amount of a TNF-α antagonist in the treatment of a virus infection in a patient comprising administering to the patient a total weekly dosage of IFN-γ containing an amount of about 100 μg to about 300 μg of drug per week in divided doses administered subcutaneously qd, qod, tiw, biw, or administered substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

One embodiment provides any of the above-described methods modified to use an effective amount of INFERGEN® consensus IFN-α and a TNF-α antagonist in the treatment of HCV infection in a patient comprising administering to the patient a dosage of INFERGEN® containing an amount of about 1 μg to about 30 μg, of drug per dose of INFERGEN®, subcutaneously qd, qod, tiw, biw, qw, qow, three times per month, once monthly, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

One embodiment provides any of the above-described methods modified to use an effective amount of INFERGEN® consensus IFN-α and a TNF-α antagonist in the treatment of HCV infection in a patient comprising administering to the patient a dosage of INFERGEN® containing an amount of about 1 μg to about 9 μg, of drug per dose of INFERGEN®, subcutaneously qd, qod, tiw, biw, qw, qow, three times per month, once monthly, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of PEGylated consensus IFN-α and an effective amount of a TNF-α antagonist in the treatment of a virus infection in a patient comprising administering to the patient a dosage of PEGylated consensus IFN-α (PEG-CIFN) containing an amount of about 4 μg to about 60 μg of CIFN amino acid weight per dose of PEG-CIFN, subcutaneously qw, qow, three times per month, or monthly, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of PEGylated consensus IFN-α and an effective amount of a TNF-α antagonist in the treatment of a virus infection in a patient comprising administering to the patient a dosage of PEGylated consensus IFN-α (PEG-CIFN) containing an amount of about 18 μg to about 24 μg of CIFN amino acid weight per dose of PEG-CIFN, subcutaneously qw, qow, three times per month, or monthly, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of IFN-α 2a or 2b or 2c and an effective amount of a TNF-α antagonist in the treatment of a virus infection in a patient comprising administering to the patient a dosage of IFN-α 2a, 2b or 2c containing an amount of about 1 MU to about 20 MU of drug per dose of IFN-α 2a, 2b or 2c subcutaneously qd, qod, tiw, biw, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of IFN-α 2a or 2b or 2c and an effective amount of a TNF-α antagonist in the treatment of a virus infection in a patient comprising administering to the patient a dosage of IFN-α 2a, 2b or 2c containing an amount of about 3 MU of drug per dose of IFN-α 2a, 2b or 2c subcutaneously qd, qod, tiw, biw, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of IFN-α 2a or 2b or 2c and an effective amount of a TNF-α antagonist in the treatment of a virus infection in a patient comprising administering to the patient a dosage of IFN-α 2a, 2b or 2c containing an amount of about 10 MU of drug per dose of IFN-α 2a, 2b or 2c subcutaneously qd, qod, tiw, biw, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of PEGASYS®PEGylated IFN-α2a and an effective amount of a TNF-α antagonist in the treatment of a virus infection in a patient comprising administering to the patient a dosage of PEGASYS® containing an amount of about 90 μg to about 360 μg, of drug per dose of PEGASYS®, subcutaneously qw, qow, three times per month, or monthly, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of PEGASYS®PEGylated IFN-α2a and an effective amount of a TNF-α antagonist in the treatment of a virus infection in a patient comprising administering to the patient a dosage of PEGASYS® containing an amount of about 180 μg, of drug per dose of PEGASYS®, subcutaneously qw, qow, three times per month, or monthly, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of PEG-INTRON®PEGylated IFN-α2b and an effective amount of a TNF-α antagonist in the treatment of a virus infection in a patient comprising administering to the patient a dosage of PEG-INTRON® containing an amount of about 0.75 μg to about 3.0 μg of drug per kilogram of body weight per dose of PEG-INTRON®, subcutaneously qw, qow, three times per month, or monthly, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of PEG-INTRON®PEGylated IFN-α2b and an effective amount of a TNF-α antagonist in the treatment of a virus infection in a patient comprising administering to the patient a dosage of PEG-INTRON® containing an amount of about 1.5 μg of drug per kilogram of body weight per dose of PEG-INTRON®, subcutaneously qw, qow, three times per month, or monthly, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Combination Therapies with Other Antiviral Agents

Other agents such as inhibitors of HCV NS3 helicase are also attractive drugs for combinational therapy, and are contemplated for use in combination therapies described herein. Ribozymes such as Heptazyme™ and phosphorothioate oligonucleotides which are complementary to HCV protein sequences and which inhibit the expression of viral core proteins are also suitable for use in combination therapies described herein.

In some embodiments, the additional antiviral agent(s) is administered during the entire course of treatment with the NS3 inhibitor compound described herein, and the beginning and end of the treatment periods coincide. In other embodiments, the additional antiviral agent(s) is administered for a period of time that is overlapping with that of the NS3 inhibitor compound treatment, e.g., treatment with the additional antiviral agent(s) begins before the NS3 inhibitor compound treatment begins and ends before the NS3 inhibitor compound treatment ends; treatment with the additional antiviral agent(s) begins after the NS3 inhibitor compound treatment begins and ends after the NS3 inhibitor compound treatment ends; treatment with the additional antiviral agent(s) begins after the NS3 inhibitor compound treatment begins and ends before the NS3 inhibitor compound treatment ends; or treatment with the additional antiviral agent(s) begins before the NS3 inhibitor compound treatment begins and ends after the NS3 inhibitor compound treatment ends.

The NS3 inhibitor compound can be administered together with (i.e., simultaneously in separate formulations; simultaneously in the same formulation; administered in separate formulations and within about 48 hours, within about 36 hours, within about 24 hours, within about 16 hours, within about 12 hours, within about 8 hours, within about 4 hours, within about 2 hours, within about 1 hour, within about 30 minutes, or within about 15 minutes or less) one or more additional antiviral agents.

As non-limiting examples, any of the above-described methods featuring an IFN-α regimen can be modified to replace the subject IFN-α regimen with a regimen of monoPEG (30 kD, linear)-ylated consensus IFN-α comprising administering a dosage of monoPEG (30 kD, linear)-ylated consensus IFN-α containing an amount of 100 μg of drug per dose, subcutaneously once weekly, once every 8 days, or once every 10 days for the desired treatment duration with an NS3 inhibitor compound.

As non-limiting examples, any of the above-described methods featuring an IFN-α regimen can be modified to replace the subject IFN-α regimen with a regimen of monoPEG (30 kD, linear)-ylated consensus IFN-α comprising administering a dosage of monoPEG (30 kD, linear)-ylated consensus IFN-α containing an amount of 150 μg of drug per dose, subcutaneously once weekly, once every 8 days, or once every 10 days for the desired treatment duration with an NS3 inhibitor compound.

As non-limiting examples, any of the above-described methods featuring an IFN-α regimen can be modified to replace the subject IFN-α regimen with a regimen of monoPEG (30 kD, linear)-ylated consensus IFN-α comprising administering a dosage of monoPEG (30 kD, linear)-ylated consensus IFN-α containing an amount of 200 μg of drug per dose, subcutaneously once weekly, once every 8 days, or once every 10 days for the desired treatment duration with an NS3 inhibitor compound.

As non-limiting examples, any of the above-described methods featuring an IFN-α regimen can be modified to replace the subject IFN-α regimen with a regimen of INFERGEN® interferon alfacon-1 comprising administering a dosage of INFERGEN® interferon alfacon-1 containing an amount of 9 μg of drug per dose, subcutaneously once daily or three times per week for the desired treatment duration with an NS3 inhibitor compound.

As non-limiting examples, any of the above-described methods featuring an IFN-α regimen can be modified to replace the subject IFN-α regimen with a regimen of INFERGEN® interferon alfacon-1 comprising administering a dosage of INFERGEN® interferon alfacon-1 containing an amount of 15 μg of drug per dose, subcutaneously once daily or three times per week for the desired treatment duration with an NS3 inhibitor compound.

As non-limiting examples, any of the above-described methods featuring an IFN-γ regimen can be modified to replace the subject IFN-γ regimen with a regimen of IFN-γ comprising administering a dosage of IFN-γ containing an amount of 25 μg of drug per dose, subcutaneously three times per week for the desired treatment duration with an NS3 inhibitor compound.

As non-limiting examples, any of the above-described methods featuring an IFN-γ regimen can be modified to replace the subject IFN-γ regimen with a regimen of IFN-γ comprising administering a dosage of IFN-γ containing an amount of 50 μg of drug per dose, subcutaneously three times per week for the desired treatment duration with an NS3 inhibitor compound.

As non-limiting examples, any of the above-described methods featuring an IFN-γ regimen can be modified to replace the subject IFN-γ regimen with a regimen of IFN-γ comprising administering a dosage of IFN-γ containing an amount of 100 μg of drug per dose, subcutaneously three times per week for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of monoPEG (30 kD, linear)-ylated consensus IFN-α containing an amount of 100 μg of drug per dose, subcutaneously once weekly, once every 8 days, or once every 10 days; and (b) administering a dosage of IFN-γ containing an amount of 50 μg of drug per dose, subcutaneously three times per week; for the desired treatment duration with an NS3 inhibitor compound.

As non-limiting examples, any of the above-described methods featuring a TNF antagonist regimen can be modified to replace the subject TNF antagonist regimen with a TNF antagonist regimen comprising administering a dosage of a TNF antagonist selected from the group of: (a) etanercept in an amount of 25 mg of drug per dose subcutaneously twice per week, (b) infliximab in an amount of 3 mg of drug per kilogram of body weight per dose intravenously at weeks 0, 2 and 6, and every 8 weeks thereafter, or (c) adalimumab in an amount of 40 mg of drug per dose subcutaneously once weekly or once every 2 weeks; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of monoPEG (30 kD, linear)-ylated consensus IFN-α containing an amount of 100 μg of drug per dose, subcutaneously once weekly, once every 8 days, or once every 10 days; and (b) administering a dosage of IFN-γ containing an amount of 100 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of monoPEG (30 kD, linear)-ylated consensus IFN-α containing an amount of 150 μg of drug per dose, subcutaneously once weekly, once every 8 days, or once every 10 days; and (b) administering a dosage of IFN-γ containing an amount of 50 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of monoPEG (30 kD, linear)-ylated consensus IFN-α containing an amount of 150 μg of drug per dose, subcutaneously once weekly, once every 8 days, or once every 10 days; and (b) administering a dosage of IFN-γ containing an amount of 100 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of monoPEG (30 kD, linear)-ylated consensus IFN-α containing an amount of 200 μg of drug per dose, subcutaneously once weekly, once every 8 days, or once every 10 days; and (b) administering a dosage of IFN-γ containing an amount of 50 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of monoPEG (30 kD, linear)-ylated consensus IFN-α containing an amount of 200 μg of drug per dose, subcutaneously once weekly, once every 8 days, or once every 10 days; and (b) administering a dosage of IFN-γ containing an amount of 100 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously three times per week; and (b) administering a dosage of IFN-γ containing an amount of 25 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously three times per week; and (b) administering a dosage of IFN-γ containing an amount of 50 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously three times per week; and (b) administering a dosage of IFN-γ containing an amount of 100 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously once daily; and (b) administering a dosage of IFN-γ containing an amount of 25 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously once daily; and (b) administering a dosage of IFN-γ containing an amount of 50 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously once daily; and (b) administering a dosage of IFN-γ containing an amount of 100 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously three times per week; and (b) administering a dosage of IFN-γ containing an amount of 25 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously three times per week; and (b) administering a dosage of IFN-γ containing an amount of 50 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously three times per week; and (b) administering a dosage of IFN-γ containing an amount of 100 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously once daily; and (b) administering a dosage of IFN-γ containing an amount of 25 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously once daily; and (b) administering a dosage of IFN-γ containing an amount of 50 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously once daily; and (b) administering a dosage of IFN-γ containing an amount of 100 μg of drug per dose, subcutaneously three times per week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 100 μg of drug per dose, subcutaneously once weekly, once every 8 days, or once every 10 days; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 100 μg of drug per dose, subcutaneously once weekly, once every 8 days, or once every 10 days; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 150 μg of drug per dose, subcutaneously once weekly, once every 8 days, or once every 10 days; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 150 μg of drug per dose, subcutaneously once weekly, once every 8 days, or once every 10 days; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 200 μg of drug per dose, subcutaneously once weekly, once every 8 days, or once every 10 days; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 200 μg of drug per dose, subcutaneously once weekly, once every 8 days, or once every 10 days; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously three times per week; (b) administering a dosage of IFN-γ containing an amount of 25 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously three times per week; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously three times per week; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously once daily; (b) administering a dosage of IFN-γ containing an amount of 25 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously once daily; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously once daily; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously three times per week; (b) administering a dosage of IFN-γ containing an amount of 25 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously three times per week; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously three times per week; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously once daily; (b) administering a dosage of IFN-γ containing an amount of 25 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously once daily; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously once daily; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α and TNF antagonist combination regimen can be modified to replace the subject IFN-α and TNF antagonist combination regimen with an IFN-α and TNF antagonist combination regimen comprising: (a) administering a dosage of monoPEG (30 kD, linear)-ylated consensus IFN-α containing an amount of 100 μg of drug per dose, subcutaneously once weekly, once every 8 days, or once every 10 days; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α and TNF antagonist combination regimen can be modified to replace the subject IFN-α and TNF antagonist combination regimen with an IFN-α and TNF antagonist combination regimen comprising: (a) administering a dosage of monoPEG (30 kD, linear)-ylated consensus IFN-α containing an amount of 150 μg of drug per dose, subcutaneously once weekly, once every 8 days, or once every 10 days; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α and TNF antagonist combination regimen can be modified to replace the subject IFN-α and TNF antagonist combination regimen with an IFN-α and TNF antagonist combination regimen comprising: (a) administering a dosage of monoPEG (30 kD, linear)-ylated consensus IFN-α containing an amount of 200 μg of drug per dose, subcutaneously once weekly, once every 8 days, or once every 10 days; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α and TNF antagonist combination regimen can be modified to replace the subject IFN-α and TNF antagonist combination regimen with an IFN-α and TNF antagonist combination regimen comprising: (a) administering a dosage of INFERGEN® interferon alfacon-1 containing an amount of 9 μg of drug per dose, subcutaneously once daily or three times per week; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α and TNF antagonist combination regimen can be modified to replace the subject IFN-α and TNF antagonist combination regimen with an IFN-α and TNF antagonist combination regimen comprising: (a) administering a dosage of INFERGEN® interferon alfacon-1 containing an amount of 15 μg of drug per dose, subcutaneously once daily or three times per week; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-γ and TNF antagonist combination regimen can be modified to replace the subject IFN-γ and TNF antagonist combination regimen with an IFN-γ and TNF antagonist combination regimen comprising: (a) administering a dosage of IFN-γ containing an amount of 25 μg of drug per dose, subcutaneously three times per week; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-γ and TNF antagonist combination regimen can be modified to replace the subject IFN-γ and TNF antagonist combination regimen with an IFN-γ and TNF antagonist combination regimen comprising: (a) administering a dosage of IFN-γ containing an amount of 50 μg of drug per dose, subcutaneously three times per week; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-γ and TNF antagonist combination regimen can be modified to replace the subject IFN-γ and TNF antagonist combination regimen with an IFN-γ and TNF antagonist combination regimen comprising: (a) administering a dosage of IFN-γ containing an amount of 100 μg of drug per dose, subcutaneously three times per week; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods that includes a regimen of monoPEG (30 kD, linear)-ylated consensus IFN-α can be modified to replace the regimen of monoPEG (30 kD, linear)-ylated consensus IFN-α with a regimen of peginterferon alfa-2a comprising administering a dosage of peginterferon alfa-2a containing an amount of 180 μg of drug per dose, subcutaneously once weekly for the desired treatment duration with an NS3 inhibitor compound.

As non-limiting examples, any of the above-described methods that includes a regimen of monoPEG (30 kD, linear)-ylated consensus IFN-α can be modified to replace the regimen of monoPEG (30 kD, linear)-ylated consensus IFN-α with a regimen of peginterferon alfa-2b comprising administering a dosage of peginterferon alfa-2b containing an amount of 1.0 μg to 1.5 μg of drug per kilogram of body weight per dose, subcutaneously once or twice weekly for the desired treatment duration with an NS3 inhibitor compound.

As non-limiting examples, any of the above-described methods can be modified to include administering a dosage of ribavirin containing an amount of 400 mg, 800 mg, 1000 mg or 1200 mg of drug orally per day, optionally in two or more divided doses per day, for the desired treatment duration with an NS3 inhibitor compound.

As non-limiting examples, any of the above-described methods can be modified to include administering a dosage of ribavirin containing (i) an amount of 1000 mg of drug orally per day for patients having a body weight of less than 75 kg or (ii) an amount of 1200 mg of drug orally per day for patients having a body weight of greater than or equal to 75 kg, optionally in two or more divided doses per day, for the desired treatment duration with an NS3 inhibitor compound.

As non-limiting examples, any of the above-described methods can be modified to replace the subject NS3 inhibitor regimen with an NS3 inhibitor regimen comprising administering a dosage of 0.01 mg to 0.1 mg of drug per kilogram of body weight orally daily, optionally in two or more divided doses per day, for the desired treatment duration with the NS3 inhibitor compound.

As non-limiting examples, any of the above-described methods can be modified to replace the subject NS3 inhibitor regimen with an NS3 inhibitor regimen comprising administering a dosage of 0.1 mg to 1 mg of drug per kilogram of body weight orally daily, optionally in two or more divided doses per day, for the desired treatment duration with the NS3 inhibitor compound.

As non-limiting examples, any of the above-described methods can be modified to replace the subject NS3 inhibitor regimen with an NS3 inhibitor regimen comprising administering a dosage of 1 mg to 10 mg of drug per kilogram of body weight orally daily, optionally in two or more divided doses per day, for the desired treatment duration with the NS3 inhibitor compound.

As non-limiting examples, any of the above-described methods can be modified to replace the subject NS3 inhibitor regimen with an NS3 inhibitor regimen comprising administering a dosage of 10 mg to 100 mg of drug per kilogram of body weight orally daily, optionally in two or more divided doses per day, for the desired treatment duration with the NS3 inhibitor compound.

As non-limiting examples, any of the above-described methods featuring an NS5B inhibitor regimen can be modified to replace the subject NS5B inhibitor regimen with an NS5B inhibitor regimen comprising administering a dosage of 0.01 mg to 0.1 mg of drug per kilogram of body weight orally daily, optionally in two or more divided doses per day, for the desired treatment duration with an NS3 inhibitor compound.

As non-limiting examples, any of the above-described methods featuring an NS5B inhibitor regimen can be modified to replace the subject NS5B inhibitor regimen with an NS5B inhibitor regimen comprising administering a dosage of 0.1 mg to 1 mg of drug per kilogram of body weight orally daily, optionally in two or more divided doses per day, for the desired treatment duration with an NS3 inhibitor compound.

As non-limiting examples, any of the above-described methods featuring an NS5B inhibitor regimen can be modified to replace the subject NS5B inhibitor regimen with an NS5B inhibitor regimen comprising administering a dosage of 1 mg to 10 mg of drug per kilogram of body weight orally daily, optionally in two or more divided doses per day, for the desired treatment duration with an NS3 inhibitor compound.

As non-limiting examples, any of the above-described methods featuring an NS5B inhibitor regimen can be modified to replace the subject NS5B inhibitor regimen with an NS5B inhibitor regimen comprising administering a dosage of 10 mg to 100 mg of drug per kilogram of body weight orally daily, optionally in two or more divided doses per day, for the desired treatment duration with an NS3 inhibitor compound.

The present embodiments provide for a method of treating a hepatitis C virus infection comprising administering to a human dosages of peginterferon alfa-2a and ribavirin under a standard of care protocol (SOC) in combination with ITMN-191 or a pharmaceutically acceptable salt thereof. The chemical structure of ITMN-191 is shown below. In some embodiments, the peginterferon alfa-2a and ribavirin in combination with ITMN-191 or a pharmaceutically acceptable salt thereof are administered in combination and provide HCV RNA levels below about 43 IU/mL, below about 25 IU/mL, or below about 9.3 IU/mL after 14 days of treatment. In some embodiments, the dosage of peginterferon alfa-2a can be about 180 μg of peginterferon alfa-2a per dose, administered subcutaneously once weekly for the desired treatment duration. In some embodiments, the dosage of peginterferon alfa-2a can be an amount in the range of about 1.0 μg to about 1.5 μg of drug per kilogram of body weight per dose, subcutaneously once or twice weekly for the desired treatment duration with the ITMN-191 and the ribavarin. In some embodiments, the dosage of ribavirin can be about 400 mg, about 800 mg, about 1000 mg or about 1200 mg of drug orally per day, optionally in two or more divided doses per day, for the desired treatment duration with the peginterferon alfa-2a and ITMN-191. In some embodiments, the dosage of ribavirin can be an amount of about 1000 mg of drug orally per day for patients having a body weight of less than 75 kg or an amount of about 1200 mg of drug orally per day for patients having a body weight of greater than or equal to 75 kg, optionally in two or more divided doses per day, for the desired treatment duration with the peginterferon alfa-2a and ITMN-191.

In some embodiments, the amounts of peginterferon alfa-2a and ribavirin administered in the SOC protocol can be lowered due to combination with ITMN-191. For example, the amounts of peginterferon alfa-2a and ribavirin can be reduced below the SOC by about 10% to about 75% during the combination treatment.

### Patient Identification

In certain embodiments, the specific regimen of drug therapy used in treatment of the HCV patient is selected according to certain disease parameters exhibited by the patient, such as the initial viral load, genotype of the HCV infection in the patient, liver histology and/or stage of liver fibrosis in the patient.

Thus, some embodiments provide any of the above-described methods for the treatment of HCV infection in which the subject method is modified to treat a treatment failure patient for a duration of 48 weeks.

Other embodiments provide any of the above-described methods for HCV in which the subject method is modified to treat a non-responder patient, where the patient receives a 48 week course of therapy.

Other embodiments provide any of the above-described methods for the treatment of HCV infection in which the subject method is modified to treat a relapser patient, where the patient receives a 48 week course of therapy.

Other embodiments provide any of the above-described methods for the treatment of HCV infection in which the subject method is modified to treat a naïve patient infected with HCV genotype 1, where the patient receives a 48 week course of therapy.

Other embodiments provide any of the above-described methods for the treatment of HCV infection in which the subject method is modified to treat a naïve patient infected with HCV genotype 4, where the patient receives a 48 week course of therapy.

Other embodiments provide any of the above-described methods for the treatment of HCV infection in which the subject method is modified to treat a naïve patient infected with HCV genotype 1, where the patient has a high viral load (HVL), where “HVL” refers to an HCV viral load of greater than 2×10⁶ HCV genome copies per mL serum, and where the patient receives a 48 week course of therapy.

One embodiment provides any of the above-described methods for the treatment of an HCV infection, where the subject method is modified to include the steps of (1) identifying a patient having advanced or severe stage liver fibrosis as measured by a Knodell score of 3 or 4 and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

Another embodiment provides any of the above-described methods for the treatment of an HCV infection, where the subject method is modified to include the steps of (1) identifying a patient having advanced or severe stage liver fibrosis as measured by a Knodell score of 3 or 4 and then (2) administering to the patient the drug therapy of the subject method for a time period of about 40 weeks to about 50 weeks, or about 48 weeks.

Another embodiment provides any of the above-described methods for the treatment of an HCV infection, where the subject method is modified to include the steps of (1) identifying a patient having an HCV genotype 1 infection and an initial viral load of greater than 2 million viral genome copies per mL of patient serum and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

Another embodiment provides any of the above-described methods for the treatment of an HCV infection, where the subject method is modified to include the steps of (1) identifying a patient having an HCV genotype 1 infection and an initial viral load of greater than 2 million viral genome copies per mL of patient serum and then (2) administering to the patient the drug therapy of the subject method for a time period of about 40 weeks to about 50 weeks, or about 48 weeks.

Another embodiment provides any of the above-described methods for the treatment of an HCV infection, where the subject method is modified to include the steps of (1) identifying a patient having an HCV genotype 1 infection and an initial viral load of greater than 2 million viral genome copies per mL of patient serum and no or early stage liver fibrosis as measured by a Knodell score of 0, 1, or 2 and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

Another embodiment provides any of the above-described methods for the treatment of an HCV infection, where the subject method is modified to include the steps of (1) identifying a patient having an HCV genotype 1 infection and an initial viral load of greater than 2 million viral genome copies per mL of patient serum and no or early stage liver fibrosis as measured by a Knodell score of 0, 1, or 2 and then (2) administering to the patient the drug therapy of the subject method for a time period of about 40 weeks to about 50 weeks, or about 48 weeks.

Another embodiment provides any of the above-described methods for the treatment of an HCV infection, where the subject method is modified to include the steps of (1) identifying a patient having an HCV genotype 1 infection and an initial viral load of less than or equal to 2 million viral genome copies per mL of patient serum and then (2) administering to the patient the drug therapy of the subject method for a time period of about 20 weeks to about 50 weeks, or about 24 weeks to about 48 weeks, or about 30 weeks to about 40 weeks, or up to about 20 weeks, or up to about 24 weeks, or up to about 30 weeks, or up to about 36 weeks, or up to about 48 weeks.

Another embodiment provides any of the above-described methods for the treatment of an HCV infection, where the subject method is modified to include the steps of (1) identifying a patient having an HCV genotype 1 infection and an initial viral load of less than or equal to 2 million viral genome copies per mL of patient serum and then (2) administering to the patient the drug therapy of the subject method for a time period of about 20 weeks to about 24 weeks.

Another embodiment provides any of the above-described methods for the treatment of an HCV infection, where the subject method is modified to include the steps of (1) identifying a patient having an HCV genotype 1 infection and an initial viral load of less than or equal to 2 million viral genome copies per mL of patient serum and then (2) administering to the patient the drug therapy of the subject method for a time period of about 24 weeks to about 48 weeks.

Another embodiment provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

Another embodiment provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of about 20 weeks to about 50 weeks, or about 24 weeks to about 48 weeks, or about 30 weeks to about 40 weeks, or up to about 20 weeks, or up to about 24 weeks, or up to about 30 weeks, or up to about 36 weeks, or up to about 48 weeks.

Another embodiment provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of about 20 weeks to about 24 weeks.

Another embodiment provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of at least about 24 weeks.

Another embodiment provides any of the above-described methods for the treatment of an HCV infection, where the subject method is modified to include the steps of (1) identifying a patient having an HCV genotype 1 or 4 infection and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

Another embodiment provides any of the above-described methods for the treatment of an HCV infection, where the subject method is modified to include the steps of (1) identifying a patient having an HCV infection characterized by any of HCV genotypes 5, 6, 7, 8 and 9 and then (2) administering to the patient the drug therapy of the subject method for a time period of about 20 weeks to about 50 weeks.

Another embodiment provides any of the above-described methods for the treatment of an HCV infection, where the subject method is modified to include the steps of (1) identifying a patient having an HCV infection characterized by any of HCV genotypes 5, 6, 7, 8 and 9 and then (2) administering to the patient the drug therapy of the subject method for a time period of at least about 24 weeks and up to about 48 weeks.

### Subjects Suitable for Treatment

Any of the above treatment regimens can be administered to individuals who have been diagnosed with an HCV infection. Any of the above treatment regimens can be administered to individuals who have failed previous treatment for HCV infection (“treatment failure patients,” including non-responders and relapsers).

Individuals who have been clinically diagnosed as infected with HCV are of particular interest in many embodiments. Individuals who are infected with HCV are identified as having HCV RNA in their blood, and/or having anti-HCV antibody in their serum. Such individuals include anti-HCV ELISA-positive individuals, and individuals with a positive recombinant immunoblot assay (MBA). Such individuals may also, but need not, have elevated serum ALT levels.

Individuals who are clinically diagnosed as infected with HCV include naïve individuals (e.g., individuals not previously treated for HCV, particularly those who have not previously received IFN-α-based and/or ribavirin-based therapy) and individuals who have failed prior treatment for HCV (“treatment failure” patients). Treatment failure patients include non-responders (i.e., individuals in whom the HCV titer was not significantly or sufficiently reduced by a previous treatment for HCV, e.g., a previous IFN-α monotherapy, a previous IFN-α and ribavirin combination therapy, or a previous pegylated IFN-α and ribavirin combination therapy); and relapsers (i.e., individuals who were previously treated for HCV, e.g., who received a previous IFN-α monotherapy, a previous IFN-α and ribavirin combination therapy, or a previous pegylated IFN-α and ribavirin combination therapy, whose HCV titer decreased, and subsequently increased).

In particular embodiments of interest, individuals have an HCV titer of at least about 10⁵, at least about 5×10⁵, or at least about 10⁶, or at least about 2×10⁶, genome copies of HCV per milliliter of serum. The patient may be infected with any HCV genotype (genotype 1, including 1a and 1b, 2, 3, 4, 6, etc. and subtypes (e.g., 2a, 2b, 3a, etc.)), particularly a difficult to treat genotype such as HCV genotype 1 and particular HCV subtypes and quasispecies.

Also of interest are HCV-positive individuals (as described above) who exhibit severe fibrosis or early cirrhosis (non-decompensated, Child's-Pugh class A or less), or more advanced cirrhosis (decompensated, Child's-Pugh class B or C) due to chronic HCV infection and who are viremic despite prior anti-viral treatment with IFN-α-based therapies or who cannot tolerate IFN-α-based therapies, or who have a contraindication to such therapies. In particular embodiments of interest, HCV-positive individuals with stage 3 or 4 liver fibrosis according to the METAVIR scoring system are suitable for treatment with the methods described herein. In other embodiments, individuals suitable for treatment with the methods of the embodiments are patients with decompensated cirrhosis with clinical manifestations, including patients with far-advanced liver cirrhosis, including those awaiting liver transplantation. In still other embodiments, individuals suitable for treatment with the methods described herein include patients with milder degrees of fibrosis including those with early fibrosis (stages 1 and 2 in the METAVIR, Ludwig, and Scheuer scoring systems; or stages 1, 2, or 3 in the Ishak scoring system).

### Preparation of NS3 Inhibitors

### Methodology

The HCV protease inhibitors in the following sections can be prepared according to the procedures and schemes shown in each section. The numberings in each of the following Preparation of NS3 Inhibitor sections including the General Method or General Procedure designations, are meant for that specific section only, and should not be construed or confused with the same numberings, if any, in other sections.

### Preparation of Ns3 Inhibitors: Section I

### Example 1

### 1.1 Preparation of 2-(4-isopropylthiazol-2-yl)-4-cloro-7-methoxy-8-methyl-quinoline (1)

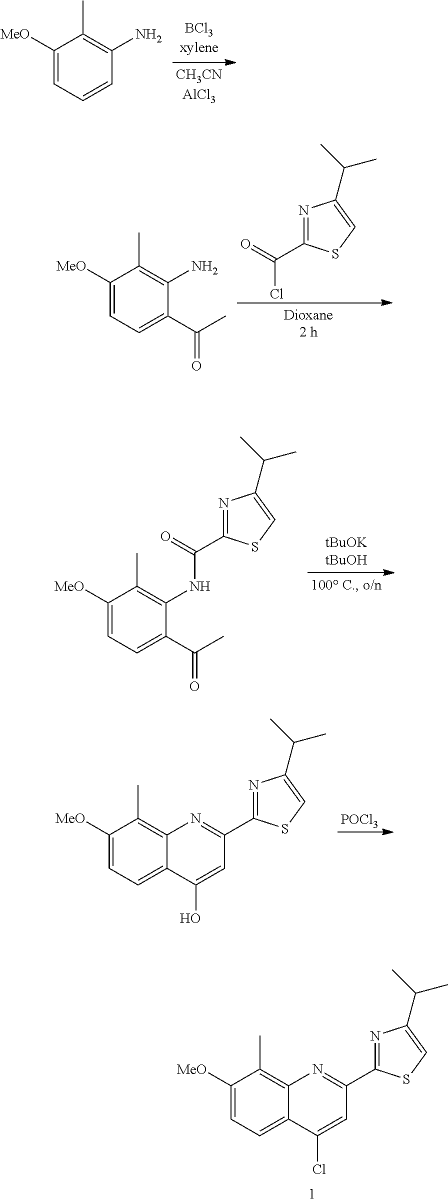

Optionally substituted 2-(thiazol-2-yl)-4-chloro-7-alkoxy-8-alkyl-quinoline 2-phenyl-4-chloro-7-alkoxy-quinolines, such as 2-(4-isopropylthiazol-2-yl)-4-chloro-7-methoxy-8-methyl-quinoline (1), can be synthesized as shown above. 3-Alkoxy-2-alkyl-anilines, such as 3-methoxy-2-methyl-aniline, can react with acetonitrile (CH₃CN) in the presence of Lewis acids, for example boron trichloride and aluminum trichloride, to provide 2-alkyl-3-alkoxy-6-acetyl-anilines such as 2-methyl-3-methoxy-6-acetyl-aniline. The 2-alkyl-3-alkoxy-6-acetyl-anilines, such as 2-methyl-3-methoxy-6-acetyl-aniline, can be coupled to an optionally substituted thiazole-2-carboxylic acid chloride, such as 4-isopropylthiazole-2-carbonyl chloride to provide an optionally substituted 1-acetyl-2-[(thiazol-2-yl)-carbonylamino]-3-alkyl-4-alkoxy-benzene, such as 1-acetyl-2-[(4-isopropyl-thiazol-2-yl)-carbonylamino]-3-methyl-4-methoxy-benzene. The optionally substituted 1-acetyl-2-[(thiazol-2-yl)-carbonylamino]-3-alkyl-4-alkoxy-benzene, such as 1-acetyl-2-[(4-isopropyl-thiazol-2-yl)-carbonylamino]-3-methyl-4-methoxy-benzene, can be cyclized under basic conditions, for example sodium tert-butoxide in tert-butanol, to provide an optionally substituted 2-(thiazol-2-yl)-4-hydroxy-7-alkoxy-8-alkyl-quinoline, such as 2-(4-isopropylthiazol-2-yl)-4-hydroxy-7-methoxy-8-methyl-quinoline. Finally, an optionally substituted 2-(thiazol-2-yl)-4-hydroxy-7-alkoxy-8-alkyl-quinoline, such as 2-(4-isopropylthiazol-2-yl)-4-hydroxy-7-methoxy-8-methyl-quinoline can be reacted with a chlorinating agent, for example phosphorous oxychloride, oxalyl chloride, thionyl chloride and the like, to provide an optionally substituted 2-(thiazol-2-yl)-4-chloro-7-alkoxy-8-alkyl-quinoline 2-phenyl-4-chloro-7-alkoxy-quinolines, such as 2-(4-isopropylthiazol-2-yl)-4-chloro-7-methoxy-8-methyl-quinoline.

Preparation of 2-Methyl-3-methoxy-6-acetyl-aniline: Boron trichloride (1M solution in dichloromethane, 31.4 mL, 31.4 mmol., 1.05 eq.) was added dropwise, over 20 minutes, at 0° C., to a solution of 3-methoxy-2-methyl-aniline (4.10 g, 29.9 mmol., 1.0 eq.) in xylenes (48 mL). The reaction mixture was stirred for 30 minutes at 0° C., then acetonitrile (4.06 mL, 77.71 mmol., 2.6 eq.) was added dropwise keeping the reaction mixture in the range 0-10° C. Stirring was continued for a further 30 minutes keeping the temperature bellow 10° C. The reaction mixture was transferred to a dropping funnel, using dichloromethane (20 mL) to rinse the initial reaction flask. This solution was added dropwise to a stirred suspension of aluminium trichloride (4.18 g, 31.38 mmol., 1.05 eq.) in dichloromethane (10 mL) at 0° C. The resulting reaction mixture was then heated under reflux for 15 hours. The reaction mixture was cooled to 0° C. and ice cold 2M hydrochloric acid (120 mL) was slowly added giving a light yellow suspension. The suspension was then stirred at 80° C. for around 90 minutes until a clear yellow solution was obtained. The reaction mixture was left to cool to ambient temperature and extracted with dichloromethane (3×100 mL). The organic extracts were combined, dried over sodium sulphate, filtered and the solvent removed under vacuum. The obtained solid was washed with diethyl ether (2×5 mL) and collected by filtration to give 2.31 g (43%) of the title compound as a beige solid. ¹H NMR (250 MHz, CDCl₃) δ ppm 7.66 (d, J=8.98 Hz, 1H), 6.45 (br. s, 2H), 6.31 (d, J=9.14 Hz, 1H), 3.88 (s, 3H), 2.55 (s, 3H), 2.02 (s, 3H). LC-MS: 97% (UV), t_{R} 1.16 min, m/z [M+1]⁺ 180.10.

Preparation of 1-Acetyl-2-[(4-isopropyl-thiazol-2-yl)-carbonylamino]-3-methyl-4-methoxy-benzene: Oxalyl chloride (5.71 g, 45 mmol., 3.0 eq) was added dropwise, at ambient temperature, to a solution of 4-isopropyl-thiazole-2-carboxylic acid (3.85 g, 22.5 mmol., 1.5 eq) in toluene (40 mL). Stirring was continued at ambient temperature until the bubbling stopped. The reaction mixture was then heated under reflux for a further 1 hour. LCMS analysis of an aliquot quenched with methanol revealed full conversion of the acid to the acid chloride. The reaction mixture was left to cool to ambient temperature and the solvent removed under vacuum. The residue was diluted with dry dioxane (40 mL). Diisopropylethylamine (3.9 g, 30 mmol., 2 eq.) was added dropwise followed by 2-methyl-3-methoxy-6-acetyl-aniline (2.7 g, 15.0 mmol., 1.0 eq). The reaction mixture was stirred at ambient temperature for 15 hours. LCMS analysis showed full conversion of the starting material to product. The solvent was removed under vacuum and the residue dissolved with ethyl acetate (75 mL). The organic layer was washed with saturated aqueous sodium hydrogen carbonate (50 mL), water (50 mL), and brine (50 mL), dried over sodium sulphate, filtered and the solvent removed under vacuum. The residue was purified by flash column chromatography using a gradient of heptanes:ethyl acetate (4:1 to 6:4). The relevant fractions were combined and the solvent removed under vacuum to give 4.55 g (91%) of the title compound as a pale yellow solid. ¹H NMR (500 MHz, CDCl₃) □ 11.28 (br. s, 1H), 7.76 (d, J=8.70 Hz, 1H), 7.17 (s, 1H), 6.79 (d, J=8.70 Hz, 1H), 3.94 (s, 3H), 3.23 (spt, J=6.89 Hz, 1H), 2.59 (s, 3H), 2.17 (s, 3H), 1.42 (d, J=6.87 Hz, 6H). LC-MS: 99% (UV), t_{R} 2.24 min, m/z [M+1]+ 333.05.

Preparation of 2-(4-isopropylthiazol-2-yl)-4-hydroxy-7-methoxy-8-methyl-quinoline: Sodium tert-butoxide (3.20 g, 28.6 mmol., 2.1 eq.) was added portion wise, at ambient temperature, to a solution of 1-acetyl-2-[(4-isopropyl-thiazol-2-yl)-carbonylamino]-3-methyl-4-methoxy-benzene (4.52 g, 13.6 mmol., 1.0 eq.) in dry tert-butanol (45 mL). The reaction mixture was stirred at 90° C. for 4 hours. LCMS analysis showed the reaction to be complete. The reaction mixture was left to cool to ambient temperature and then diluted with ethyl acetate (100 mL). The organic layer was washed with 1M aqueous potassium hydrogen sulphate (75 mL), water (50 mL), brine (50 mL), dried over sodium sulphate, filtered and the solvent removed under vacuum to give 4.63 g (99%) of the title compound as an off white solid. ¹H NMR (500 MHz, CDCl₃) □ 9.59 (br. s, 1H), 8.26 (d, J=9.16 Hz, 1H), 7.10 (s, 1H), 7.03 (d, J=9.16 Hz, 1H), 6.77 (s, 1H), 3.98 (s, 3H), 3.20 (spt, J=6.87 Hz, 1H), 2.43 (s, 3H), 1.39 (d, J=7.02 Hz, 6H). LC-MS: 95% (UV), t_{R} 2.24 min, m/z [M+1]+ 315.15.

Preparation of 2-(4-isopropylthiazol-2-yl)-4-chloro-7-methoxy-8-methyl-quinoline (1): 2-(4-isopropylthiazol-2-yl)-4-hydroxy-7-methoxy-8-methyl-quinoline (4.63 g, 13.6 mmol., 1.0 eq.) was charged into a 100 mL round bottom flask. Phosphorous oxychloride (45 mL) was added and the reaction mixture stirred at 90° C. for 3 hours. Monitoring the reaction mixture by ¹H NMR showed full consumption of the starting material. The reaction mixture was left to cool to ambient temperature and the solvent removed under vacuum. The residue was diluted with ethyl acetate (80 mL) and the reaction mixture cooled to 0° C. 2M aqueous sodium hydroxide solution was added portion wise until the pH of the aqueous phase was 14 (stir reaction mixture for 1 min between every NaOH addition). The two layers were separated and the organic layer was further washed with water (50 mL) and brine (50 mL). The organic layer was dried over sodium sulphate, filtered and the solvent removed under vacuum to give 4.11 g (91%) of the title compound 1 as a pale brown solid. ¹H NMR (500 MHz, CDCl₃) □ 8.28 (s, 1H), 8.09 (d, J=9.16 Hz, 1H), 7.38 (d, J=9.16 Hz, 1H), 7.06 (s, 1H), 4.02 (s, 3H), 3.20 (spt, J=6.87 Hz, 1H), 2.73 (s, 3H), 1.40 (d, J=6.87 Hz, 6H).

### 1.2 Synthesis of Macrocyclic Precursors

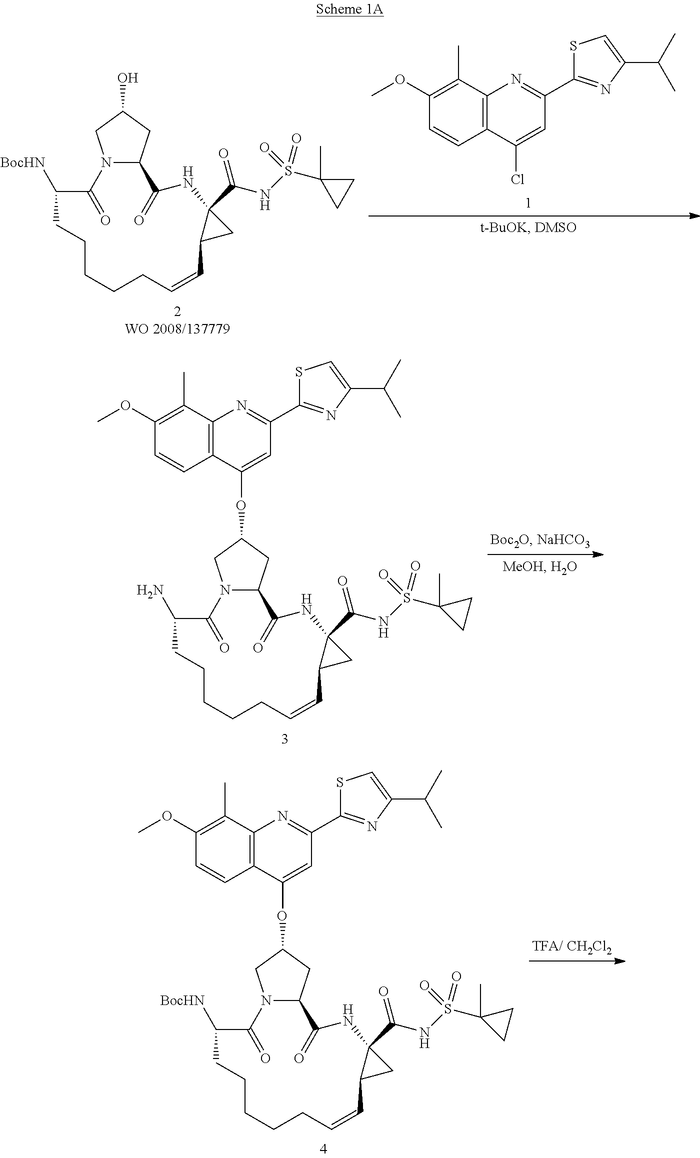

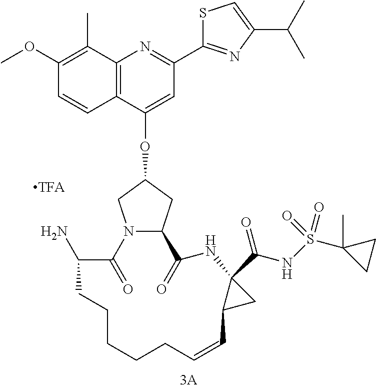

Compound 2 was synthesized according to WO 2008/137779. To a solution of compound 2 (1.56 g, 2.67 mmol) in 30 mL of DMSO was added t-BuOK (1.5 g, 13.35 mmol) in portions at ambient temperature, then the mixture was stirred for 15 min at ambient temperature. After that, compound 1 (1.065 g, 3.2 mmol) was added, the resulting mixture was stirred at 30° C. for 12 h, the reaction was monitored by LC-MS. After completion of the reaction, the mixture was cooled by ice water, quenched by addition of ice-water (2 mL). Then the mixture was extracted with ethyl acetate (50 mL×3), the aqueous layer was acidified to pH=6 and extracted with ethyl acetate (30 mL×3), the organic layers were combined, washed by brine, dried over anhydrous sodium sulfate, solvent was removed under reduced pressure to afford the crude product compound 3.

To a solution of crude compound 3 (2 g, 2.67 mmol) in 30 mL of MeOH and water (1 mL) was added Boc₂O (873 mg, 4.0 mmol) and NaHCO3 (672 mg, 8.0 mmol) in portions at ambient temperature, then the mixture was stirred for 2 hrs at ambient temperature. After completion of the reaction, the solvent was evaporated and the residue was purified with flash chromatography (petroleum ether:ethyl acetate=1:1) to afford compound 4 (1.55 g, 66%).

To a solution of compound 4 (1.55 g, 1.76 mmol) in 6 mL of CH₂Cl₂ was added 3 mL of TFA. The resulting mixture was stirred at room temperature for 2 h. After that, the solvent was evaporated, the mixture was diluted with ethyl acetate (150 mL), washed with saturated aqueous NaHCO₃, the organic layer was dried over anhydrous sodium sulfate, solvent was removed under reduced pressure to afford compound 3A (1.3 g, 95%).

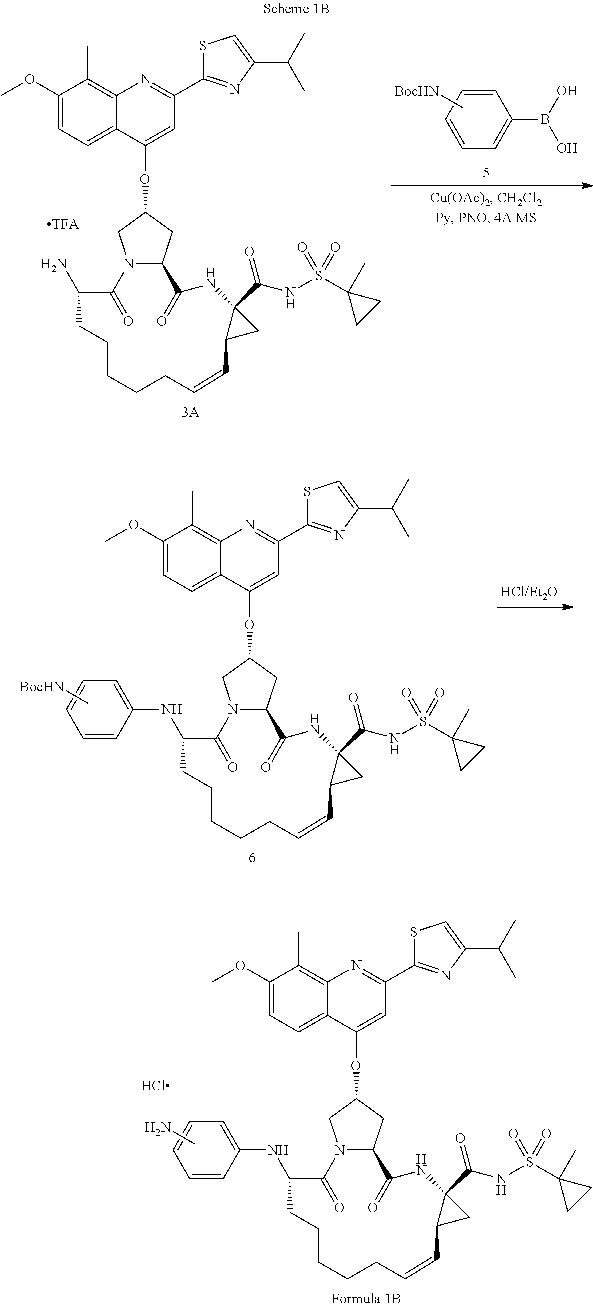

A mixture of compound 3A (1 eq.), substituted phenyl boronic acid 5 (3 eq.), Cu(OAc)₂ (2 eq.), pyridine (10 eq.), pyridine N-Oxide (1 eq.) and molecular sieves 4 Å in dichloromethane (4 mL) was stirred at room temperature under oxygen atmosphere. The reaction was monitored by LC-MS. After completion of the reaction, the solid was removed by filtration, the solvent was removed and the crude mixture was purified by prep-TLC or prep-HPLC to give compound 6. The solution of compound 6 in a solution of HCl/Et₂O (5 mL) was stirred at 0° C. protected by nitrogen for 1.5 h. The resulting mixture was dried by vacuum to give a compound of Formula 1B.

### 1.3 Synthesis of Compound 101

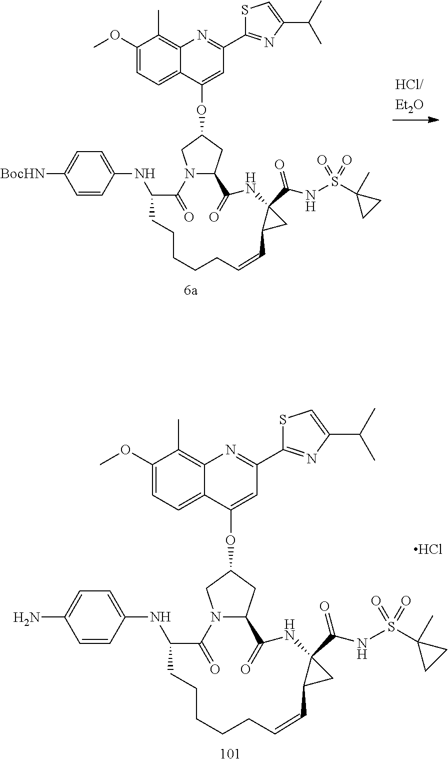

The solution of compound 6a in a solution of HCl/Et₂O (5 mL) was stirred at 0° C. protected by nitrogen for 1.5 h. The resulting mixture was dried by vacuum to give the title compound 101. 5 mg, 46%. MS (ESI) m/z (M+H)⁺ 870.2.

### 1.4 Synthesis of Compound 102

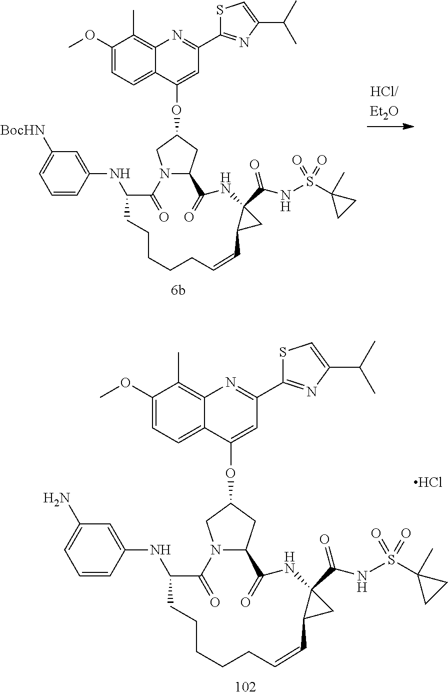

Compound 102 was prepared using the procedure similar to that of compound 101. 6 mg, 46%. MS (ESI) m/z (M+H)⁺ 870.2.

### 1.5 Synthesis of Compound 103

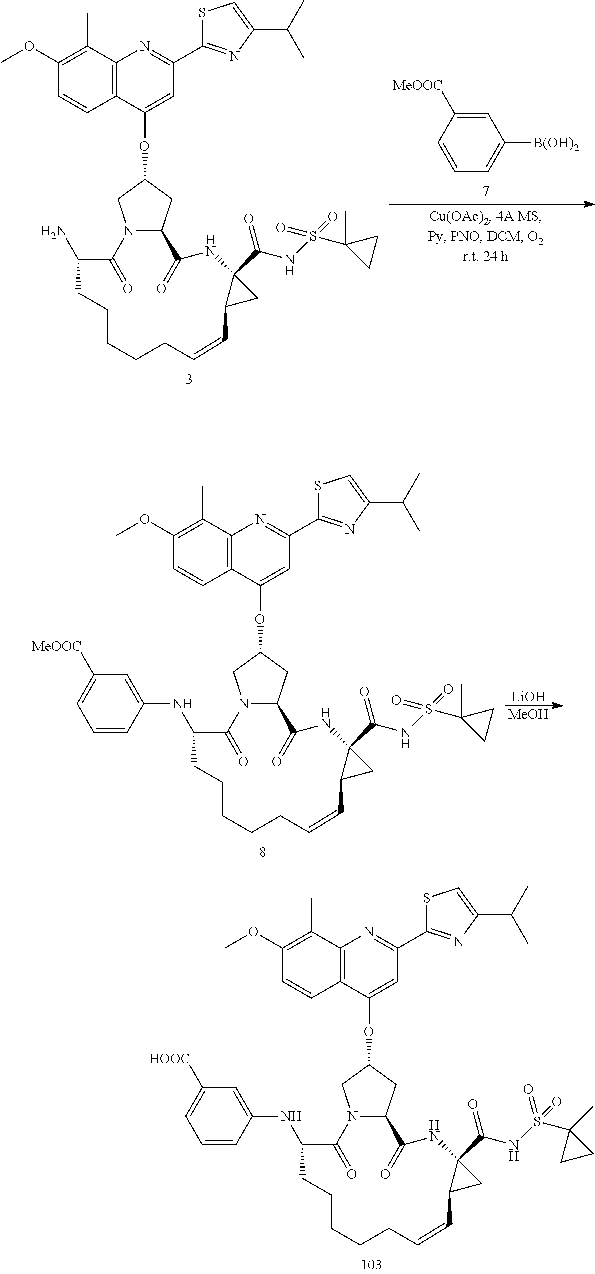

A mixture of compound 3 (400 mg, 0.52 mmol.), boronic acid 7 (276.6 mg, 1.54 mmol.), Cu(OAc)₂ (188 mg, 1.04 mmol.), pyridine (410.8 mg, 5.2 mmol.), pyridine N-Oxide (247 mg, 2.6 mmol.) and molecular sieves 4A in dichloromethane (20 mL) was stirred for 24 h at room temperature under oxygen atmosphere, The reaction was monitored by TLC. After completion of the reaction, the solid was filtered and the crude mixture was purified by column to give the crude compound 8 (800 mg, purity 20%).

Compound 8 (800 mg, purity 20%) was dissolved in 10 mL of methanol, LiOH (240 mg) and 2 mL of water were added, the resulting mixture was heated to reflux overnight, after completion of the reaction, the mixture was cooled by ice water, 2 M HCl was added to acidify the mixture to pH=3-4, then the mixture was extracted with EtOAc, the organic layers were combined, washed with brine, dried over anhydrous Na₂SO₄, the solvent was removed under reduced pressure, the crude was purified with prep-HPLC, 120 mg of compound 103 was obtained. MS (ESI) m/e (M+H⁺) 898.8.

### 1.6 Synthesis of Amide Library

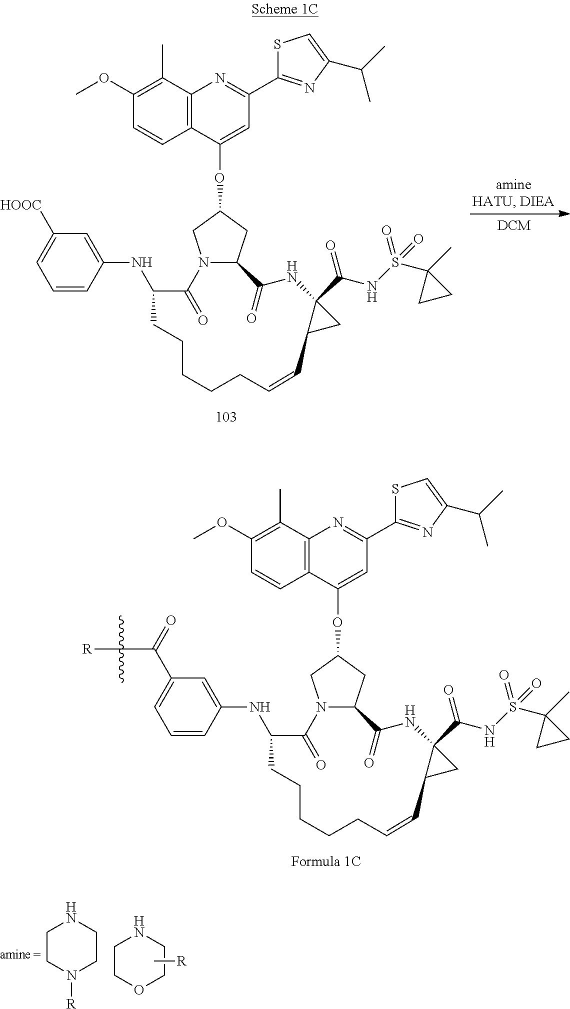

To a solution of compound 103 (1 eq) in dry DCM (5 mL) was added amine (1.5 eq.), followed by adding DIEA (5 eq) and HATU (1.8 eq), the reaction mixture was protected by nitrogen and stirred at room temperature overnight. The resulting mixture was diluted with EtOAc and washed with water. The organic layer was dried and concentrated to give residue. The residue was purified by prep-HPLC to afford final compound Formula 1C.

The following compounds were prepared using the above procedure.

TABLE 1
Compounds prepared according to Scheme 1C.

| Compound | Structure | Yield |
|---|---|---|
| 104 | 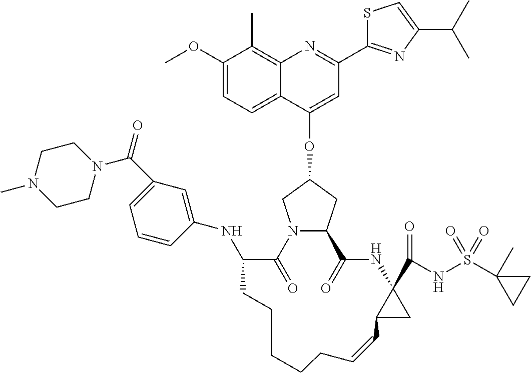 | 28.1 mg, 26%. MS (ESI) m/z (M + H)⁺ 981.1 |
| 105 | 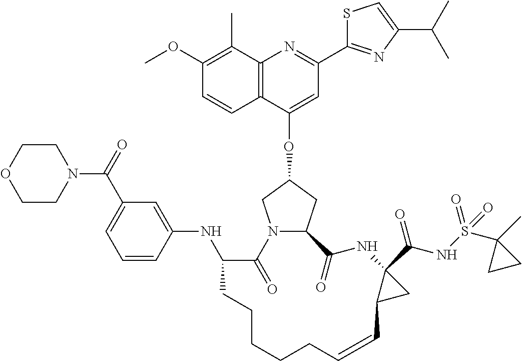 | 60 mg, 62%. MS (ESI) m/z (M + H)⁺ 968 |
| 106 | 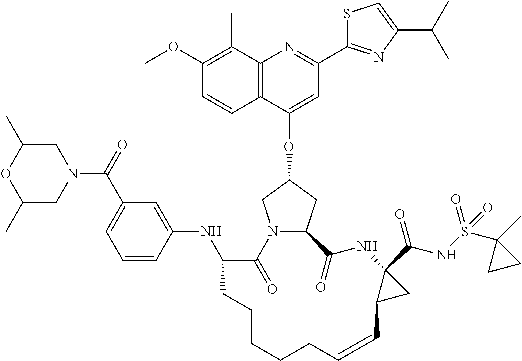 | 10 mg, 45%. MS (ESI) m/z (M + H)⁺ 996.4 |
| 107 | 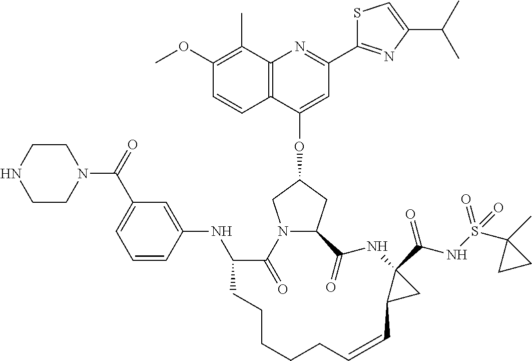 | 31 mg, 32%. MS (ESI) m/z (M + H)⁺ 967.3 |
| 108 | 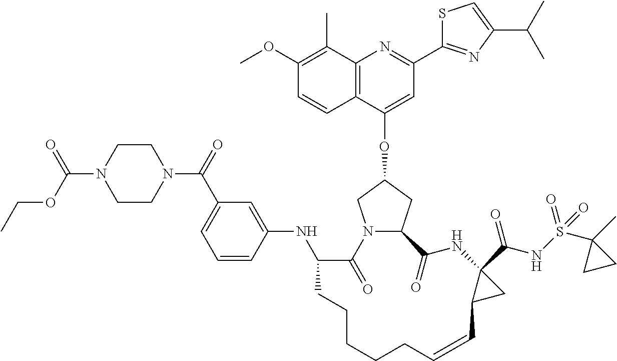 | 20 mg, 46%. MS (ESI) m/z (M + H)⁺ 1039 |
| 109 | 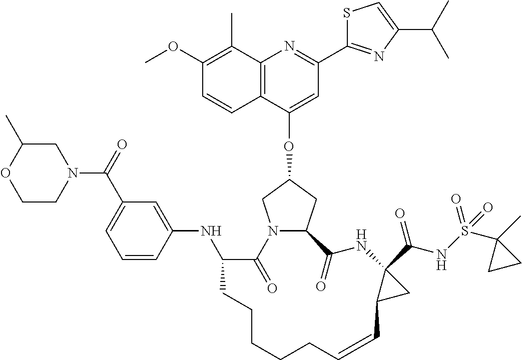 | 14 mg, 34%. MS (ESI) m/z (M + H)⁺ 982.1 |
| 110 | 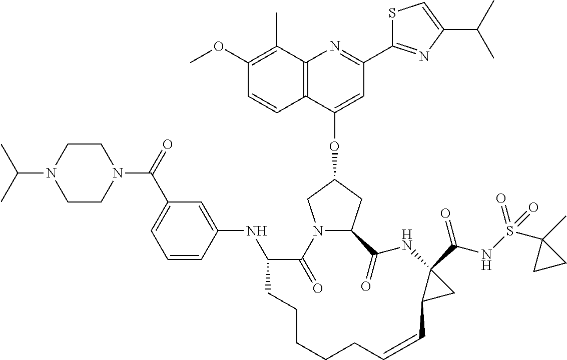 | 7 mg, 16%. MS (ESI) m/z (M + H)⁺ 1011 |
| 111 | 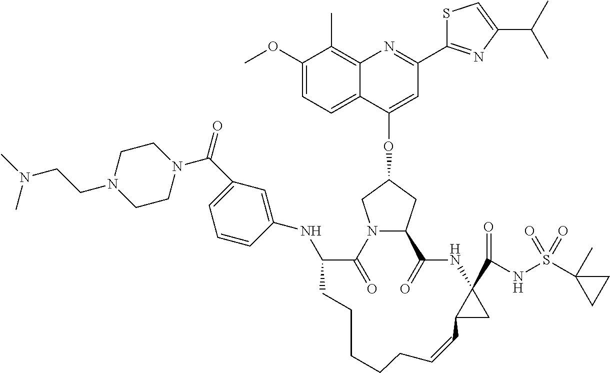 | 10.7 mg, 23%. MS (ESI) m/z (M + H)⁺ 1039 |
| 112 | 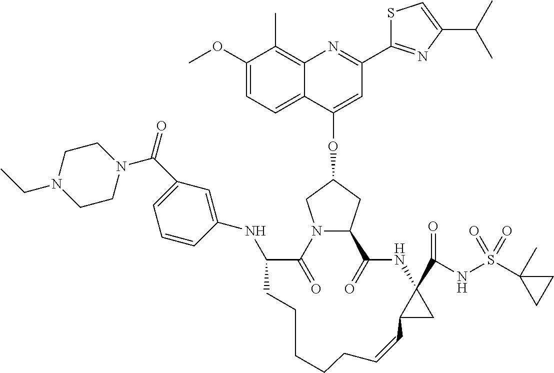 | 27.8 mg, 62%. MS (ESI) m/z (M + H)⁺ 995.1 |
| 113 | 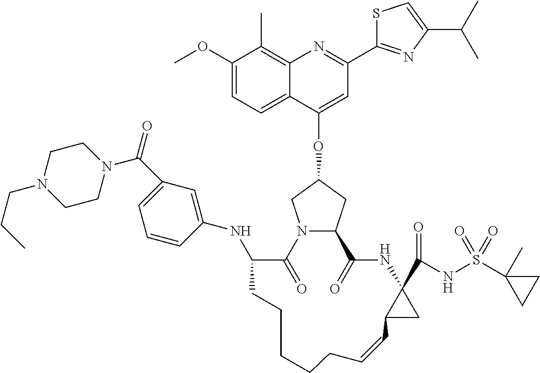 | 20.9 mg, 46%. MS (ESI) m/z (M + H)⁺ 1009 |
| 114 | 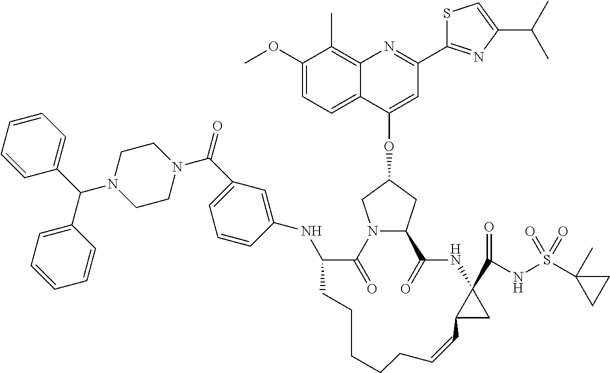 | 10.3 mg, 21%. MS (ESI) m/z (M + H)⁺ 1133 |
| 115 | 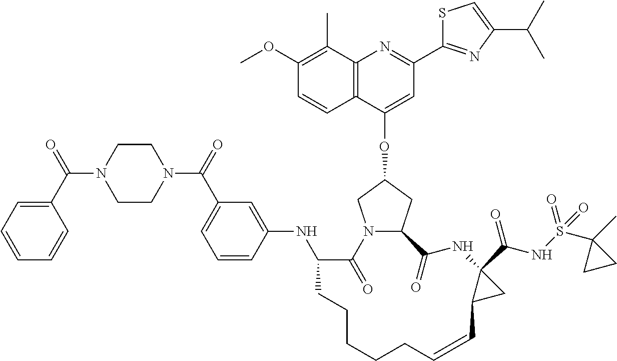 | 12 mg, 25%. MS (ESI) m/z (M + H)⁺ 1073 |
| 116 | 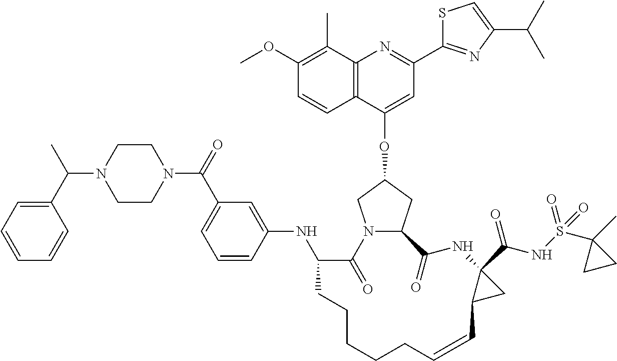 | 11 mg, 23%. MS (ESI) m/z (M + H)⁺ 1071 |
| 117 | 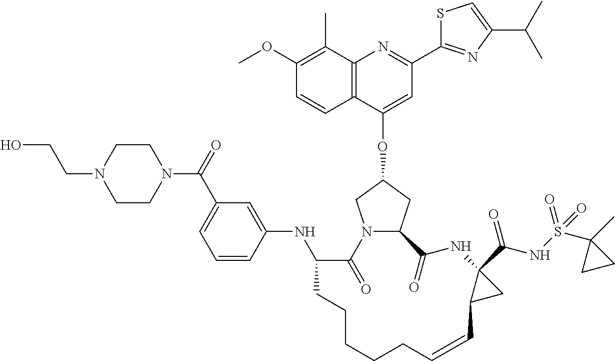 | 12 mg, 27%. MS (ESI) m/z (M + H)⁺ 1011.3 |
| 118 | 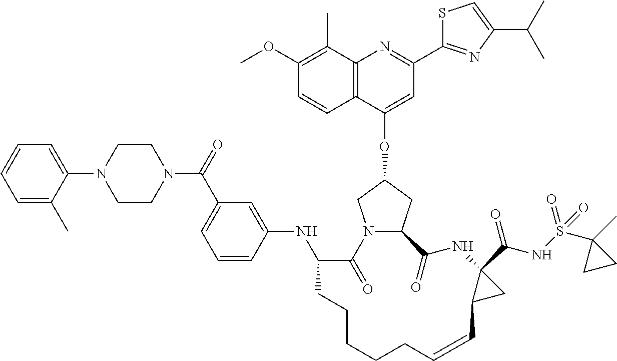 | 14 mg, 30%. MS (ESI) m/z (M + H)⁺ 1057 |
| 119 | 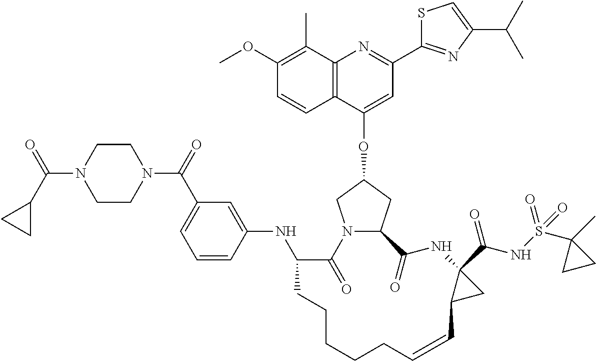 | 19 mg, 41%. MS (ESI) m/z (M + H)⁺ 1035 |
| 120 | 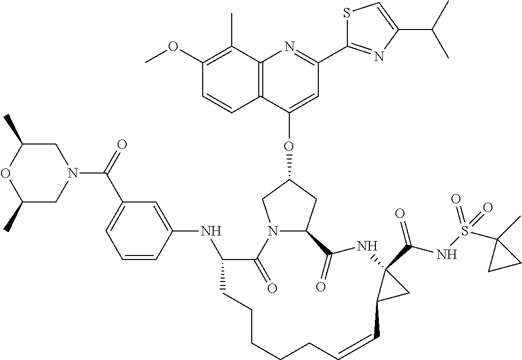 | 14 mg, 32%. MS (ESI) m/z (M + H)⁺ 996.2 |
| 121 | 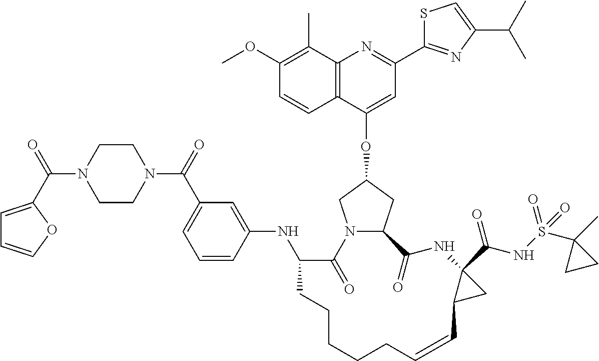 | 11 mg, 23%. MS (ESI) m/z (M + H)⁺ 1061 |
| 122 | 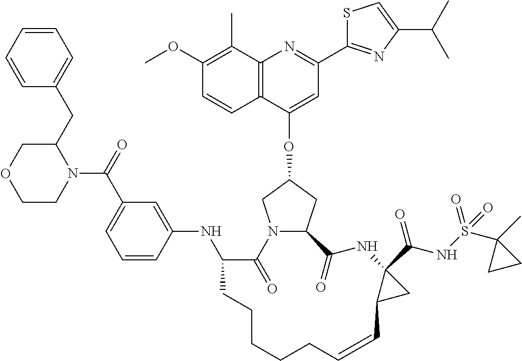 | 11 mg, 25%. MS (ESI) m/z (M + H)⁺ 1058.5 |
| 123 | 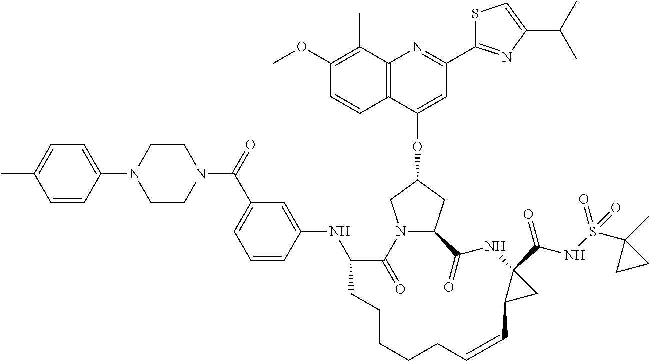 | 12 mg, 27%. MS (ESI) m/z (M + H)⁺ 1057.4 |
| 124 | 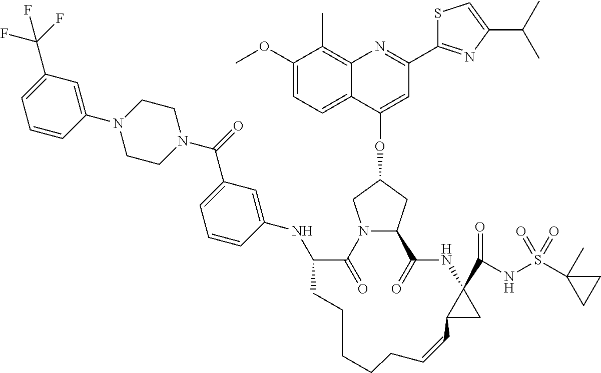 | 9.7 mg, 21%. MS (ESI) m/z (M + H)⁺ 1111.3 |
| 125 | 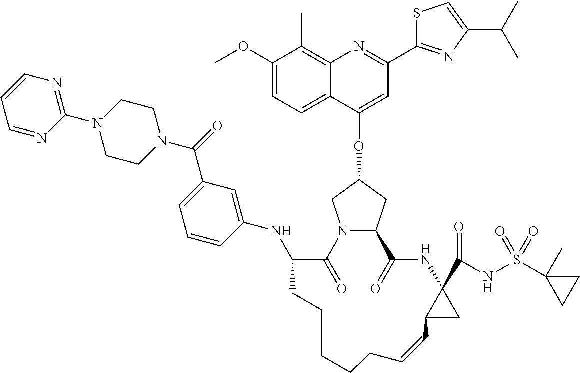 | 6.4 mg, 16%. MS (ESI) m/z (M + H)⁺ 1045.4 |
| 126 | 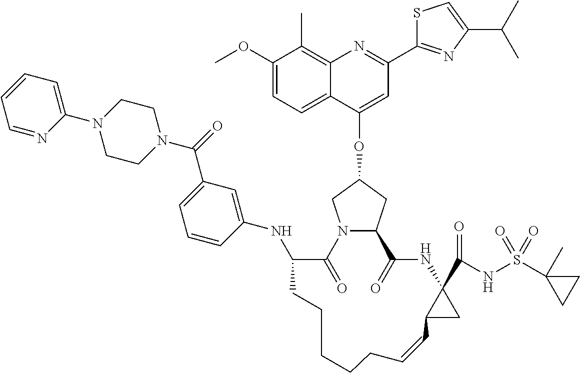 | 6.8 mg, 16%. MS (ESI) m/z (M + H)⁺ 1044.4 |

### 1.7 Synthesis of Compound 127

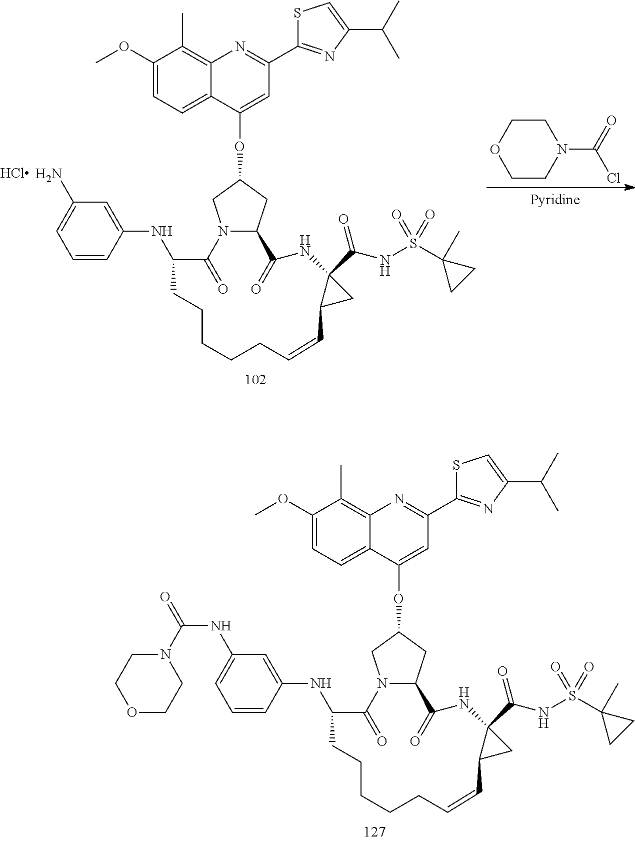

To a stirred solution of 102 (25 mg, 1 eq) in pyridine (11.5 ml, 143 mmol) was added morpholine-4-carbonyl chloride (10.2 ml, 132 mmol). The reaction solution was stirred for 2 h at 40° C. Then the reaction was quenched with water and extracted with EtOAc, the organic layer was dried and concentrated to give residue. The residue was purified by prep-HPLC to afford compound 127. 26 mg, 50%. MS (ESI) m/z (M+H)⁺ 983.1.

### 1.8 Synthesis of Compound 128

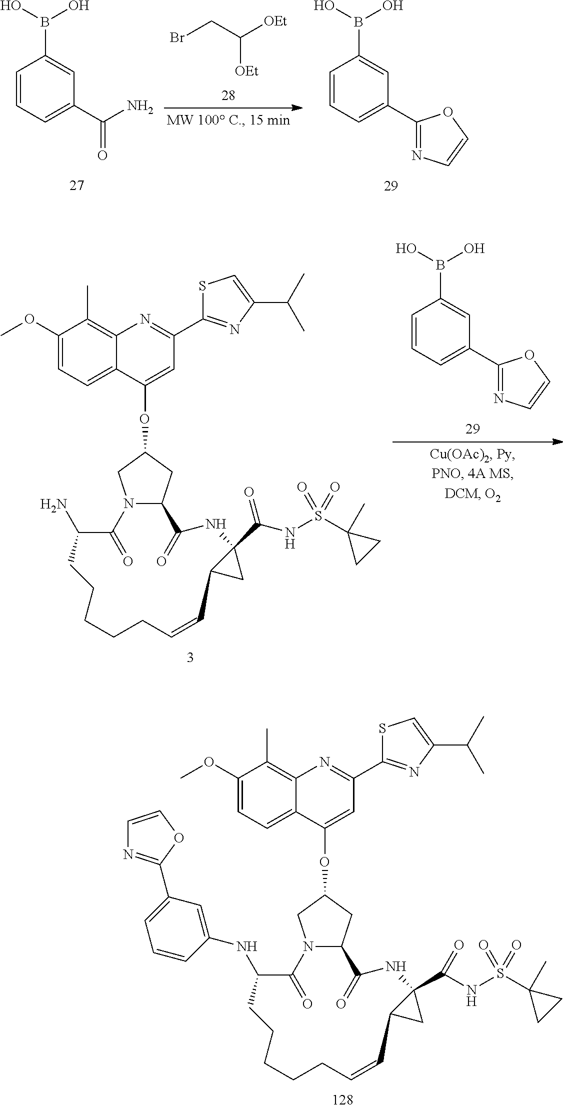

To a microwave tube was charged with compound 27 and 28, the reaction solution was heated to 100° C. for 15 min. Then the reaction was quenched with brine and extracted with EtOAc. The organic phase was dried over Na₂SO₄, filtered and dried over vacuum to give crude compound 29. The title compound was purified by prep-TLC eluted by EtOAc (230 mg, 100%). MS (ESI) m/z (M+H)⁺ 189.8. ¹H NMR: (400 MHz, DMSO-d₆) δ 8.45 (s, 1H), 8.16 (s, 1H), 8.01 (d, J=7.6 Hz, 1H), 7.92 (d, J=14.8 Hz, 1H), 7.49 (t, J=15.2 Hz, 1H), 7.37 (s, 1H), 2.86 (s, 1H), 2.65 (s, 1H).

Compound 128 was synthesized by following the first step in the synthesis of compound 103, except compound 29 was used instead of compound 7. 32 mg, 13%. MS (ESI) m/z (M+H)⁺ 922.1.

### 1.9 Synthesis of Compound 200 and 129

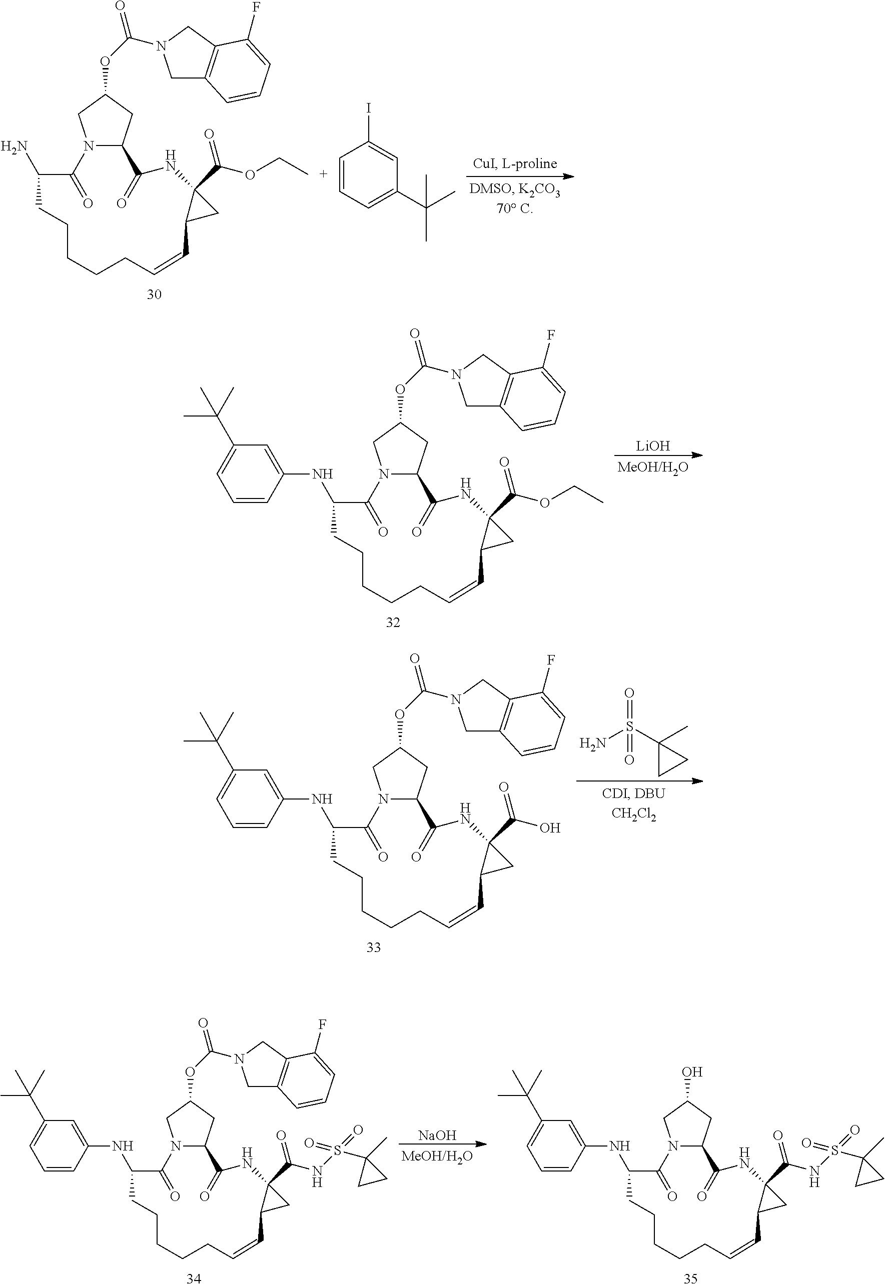

A tube (40 mL) was charged with compound 30 (850 mg, 1.5 mmol), CuI (57 mg, 0.3 mmol), L-proline (69 mg, 0.6 mmol) and K₂CO₃ (1.24 g, 9 mmol), evacuated and backfilled with argon. DMSO (10 mL) and 1-tert-butyl-3-iodobenzene 31 (1.95 g, 7.5 mmol) were added successively. The tube was sealed and heated at 70° C. for 48 hours. LCMS monitored the reaction, after material was consumed, the reaction mixture was cooled to r.t. and diluted with ethyl acetate (200 mL), filtered. The organic layer was washed with brine, dried over Na₂SO₄, concentrated in vacuo. The residue was purified with flash chromatography (petroleum ether:ethyl acetate=1:1) to afford compound 32 (350 mg, 35%).

To a solution of compound 32 (350 mg, 0.51 mmol) in methanol (20 mL) and water (1 mL) was added LiOH (144 mg, 6.0 mmol) in portions, the resulting mixture was stirred at room temperature overnight. After completion of the reaction, the solvent was evaporated, the residue was acidified by aq. HCl (1 N) to pH=5-6, then the mixture was extracted by ethyl acetate, the organic layers were combined, washed by brine, dried over anhydrous sodium sulfate, concentrated under reduced pressure to afford crude compound 33 (400 mg, 119%).

A mixture of compound 33 (350 mg crude, 0.53 mmol) and CDI (172 mg, 1.06 mmol) in 10 mL of dry CH₂Cl₂ was stirred at reflux for 2 hours under nitrogen protection. LCMS detected the intermediate formed. Then the mixture was cooled to r.t., the sulfonamide (287 mg, 2.12 mmol) and DBU (323 mg, 2.12 mmol) were added. The reaction mixture was heated at 60° C. for 15 hours. After the reaction completion, the mixture was cooled to r.t., water (10 mL) was added, acidified with aq. HCl (1 M) to pH=5-6, extracted with EtOAc (30 mL×3), washed with brine, dried over anhydrous sodium sulfate, concentrated under reduced pressure to afford crude product. It was purified with prep-TLC (PE:EA=1:1) to afford compound 34 (200 mg, 48%).

To a solution of compound 34 (200 mg, 0.257 mmol) in MeOH (10 mL) was added a solution NaOH (308 mg, 7.7 mmol) in H₂O (1.5 mL), the mixture was heated at 50° C. The reaction was monitored with LCMS. When the reaction was completed, the reaction mixture was cooled to r.t, the solvent was removed under reduced pressure. The residue was diluted with water and acidified with aq. HCl (1 M) to pH=5˜6, extracted with EtOAc (30 mL×3). The organic layers were combined and washed with brine, dried over anhydrous sodium sulfate, concentrated under reduced pressure to afford crude product compound 35. It was used directly in next step (180 mg crude, 114%).

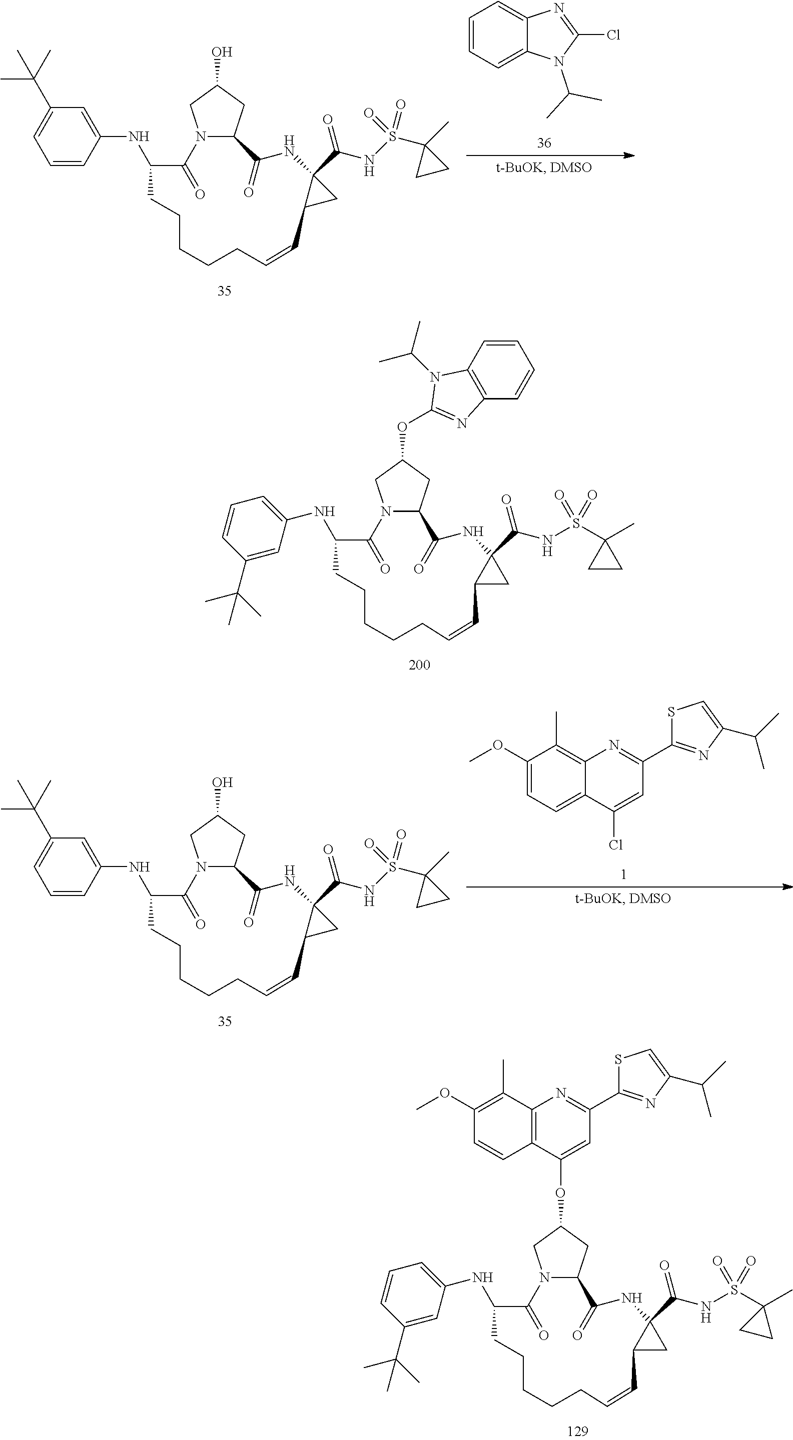

Compound 200 (26.3 mg, 12%. MS (ESI) m/z (M+H)⁺ 773.2) and 129 (6.3 mg, 9%. MS (ESI) m/z (M+H)⁺ 911.4) were prepared following the general procedure shown above.

### Example 2

### Benzoimidazole Analogs

### 2.1 Synthesis of Precursor Compound 15

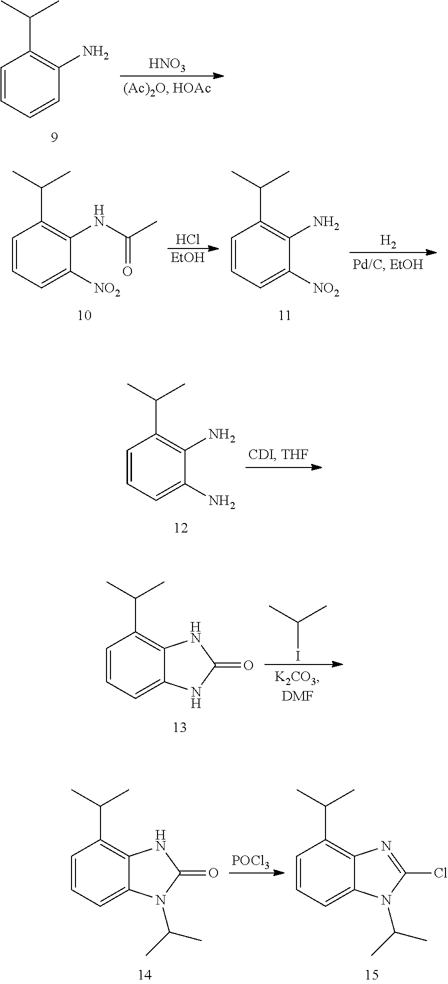

To a solution of compound 9 (5 g, 37.0 mmol) in 20 mL of acetic acid and 7 mL of acetic anhydride was added 3.1 mL of fuming nitric acid at 0° C. The solution was stirred for another additional hour, then allowed to room temperature, and continued to stir for 16 h. TLC analysis showed the reaction complete. The reaction mixture was poured into ice water and partitioned between EtOAc and water. The organic layers was washed with brine, dried over NaSO4, filtered and concentrated in vacuo to give brown oil. Purification by flash chromatography gave compound 10 as white solid (2.5 g, 30.5%).

To a stirred solution of compound 10 (2.5 g, 11.3 mmol) in 15 mL of ethanol and 20 mL of concentrated hydrochloric acid was heated at reflux for 17 h. TLC analysis showed the reaction complete. The reaction mixture was cooled to r.t. and poured into ice. The mixture was basified with aqueous 5% sodium hydroxide. The resultant solid was collected by filtration and thoroughly washed with water. Compound 11 was obtained as yellow solid (2.0 g, 98%).

To a suspension of Pd/C (0.2 g) in 10 mL of ethanol was added a solution of compound 11 (2.0 g, 11.1 mmol) in 20 mL of ethanol. The reaction mixture was stirred under hydrogen atmosphere (30 psi) at 25° C. for 16 h. TLC analysis showed the reaction complete. The mixture was filtered. The filtrate was concentrated to obtain compound 12 as brown solid (1.6 g, 96%).

To a solution of compound 12 (2 g, 13.3 mmol) in 30 mL of anhydrous THF was added (8.69 g, 53.3 mmol). The mixture was stirred for 16 h at room temperature. TLC analysis showed the reaction complete. All the volatiles were removed under reduced pressure. The residue was diluted with 10 mL of water, extracted with EtOAc. The combined organic layers were washed with brine, dried over Na₂SO₄, filtered and concentrated to give a brown solid. Purification by recrystallization in CH₂Cl₂ gave compound 13 as off-white solid (1.7 g, 72.6%).

To a solution of 13 (100 mg, 0.567 mmol) in 1 mL of DMF was added K₂CO₃ (157 mg, 1.135 mmol) and 2-Iodopropane (193 mg, 1.135 mmol). The mixture was stirred at room temperature for 16 h. TLC analysis showed the reaction complete. The mixture was diluted with 3 mL of water, extracted with EtOAc. The combined organic layers were washed with brine, dried over Na₂SO₄, filtered and concentrated to afford a brown solid. Purification by TLC gave compound 14 as a yellow solid (34 mg, 27%) ¹H NMR (400 MHz, CDCl₃) δ 11.1 (s, 1H), 6.99-7.09 (m, 3H), 4.79 (m, 1H), 3.26 (m, 1H), 1.61 (d, J=7.2 Hz, 6H), 1.38 (d, J=6.8 Hz, 6H).

The solution of 14 (290 mg, 1.33 mmol) in 4 mL of POCl₃ was heated to reflux for 16 h. TLC analysis showed the reaction completed. The mixture was poured into ice water, neutralized with saturated aqueous NaHCO₃, and then extracted with ethyl acetate (20 mL×3), the organic layers were combined, washed by brine, dried over anhydrous sodium sulfate, solvent was removed under reduced pressure to gave compound 15 (240 mg, 76%). The crude compound 15 was used directly in the synthesis of compound 201.

### 2.2 Synthesis of Precursor Compound 26

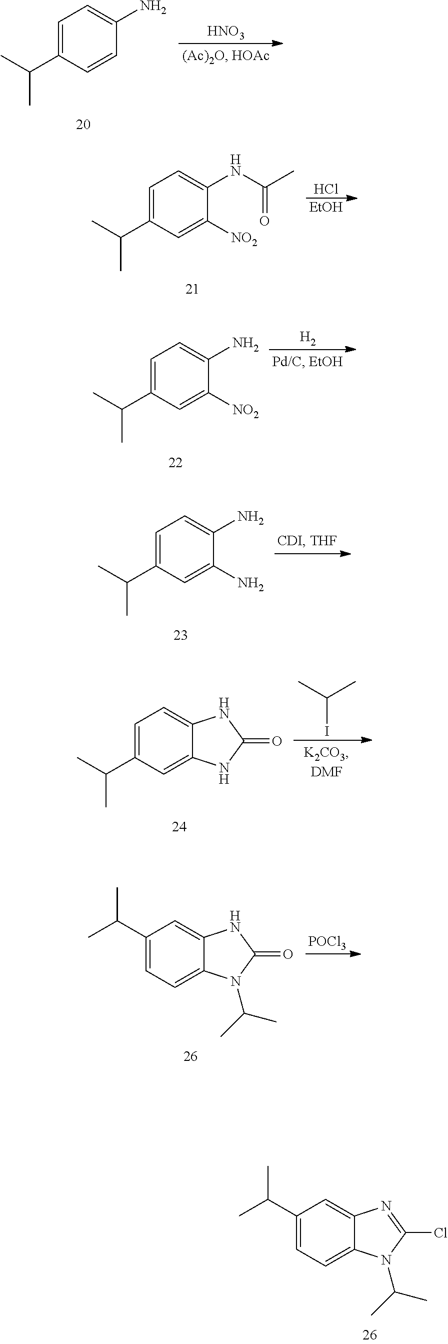

A flask was charged with compound 20 (15 g) and HOAc (63 mL) in an ice-water bath. Added Ac₂O(21 mL) in portions to maintained the temperature below 15° C. then added fuming nitric acid in portions to maintained the temperature below 15° C. After 1 hour and 30 minutes, added 600 mL of water to stop the reaction. There are yellow solid separated out, the solid was purified by recrystallization (HOAc) to give the compound 21 as a yellow solid (9.8 g, 39.8%).

A flask was charged with compound 21 (5 g, 22.5 mmol), EtOH and conc. HCl (20 mL, 33.8 mmol) under reflux. The mixture was left standing overnight. Then the mixture was added water (100 mL), alkalified by aqueous NaOH, extracted with EtOAc. Dried and concentrated to give compound 22 (3.8 g, 94%).

To a solution of compound 22 (2.2 g) in 50 mL of ethanol was added Pd/C (700 mg). the resulting mixture was stirred overnight under hydrogen atmosphere (30 psi) at r.t. TLC analysis showed the reaction complete. Then filtered and concentrated to give compound 23 (1.69 g, 92%).

To a solution of compound 23 (1.5 g, 10 mmol) in 10 mL of anhydrous TRF was added CDI (6.52 g, 40 mmol). the resulting mixture was stirred overnight at r.t. Added water (50 mL) before the reaction stopped, there was white solid separated out and filtered to get the solid product and purified by column chromatography to give the compound 24 (1.1 g, 62.5%).

To a solution of 24 (500 mg, 2.8 mmol) in 5 mL of DMF was added K₂CO₃ (579 mg, 4.2 mmol) and 2-Iodopropane (714 mg, 4.2 mmol). The mixture was stirred at room temperature for 16 h. TLC analysis showed the reaction complete. The mixture was diluted with 10 mL of water, extracted with EtOAc. The combined organic layers were washed with brine, dried over Na₂SO₄, filtered and concentrated to give a brown solid. Purification by TLC gave compound 25 as yellow solid (138 mg, 22.2%).

The solution of 25 (600 mg, 2.75 mmol) in 4 mL of POCl₃ was heated at reflux for 16 h. TLC analysis showed the reaction completed. The mixture was poured into ice water, neutralized with saturated aqueous NaHCO3, and then extracted by ethyl acetate (20 mL×3), the organic layers were combined, washed by brine, dried over anhydrous sodium sulfate, solvent was removed under reduced pressure to give compound 26 (159 mg, 24%). The crude compound 26 was used directly in the synthesis of compound 202.

### 2.3 Synthesis of Precursor Compound 46

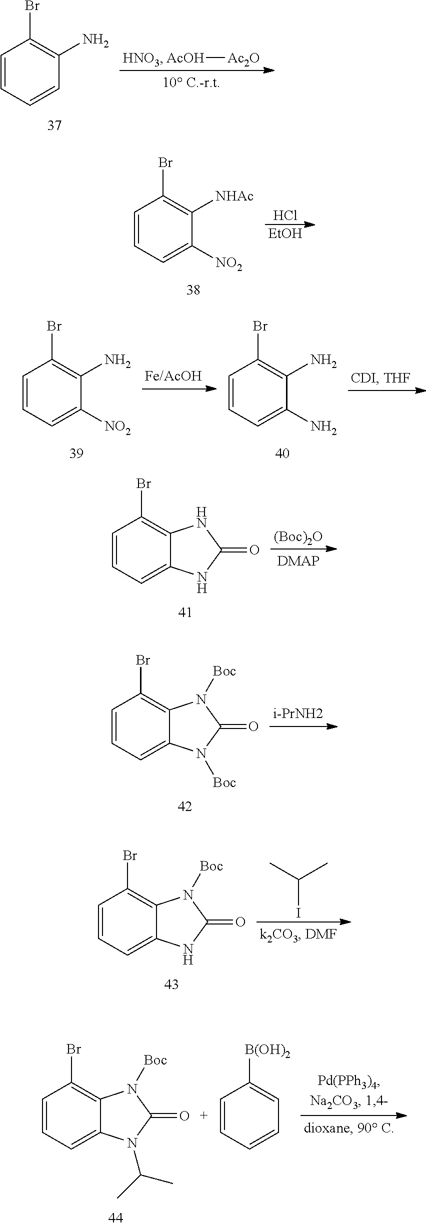

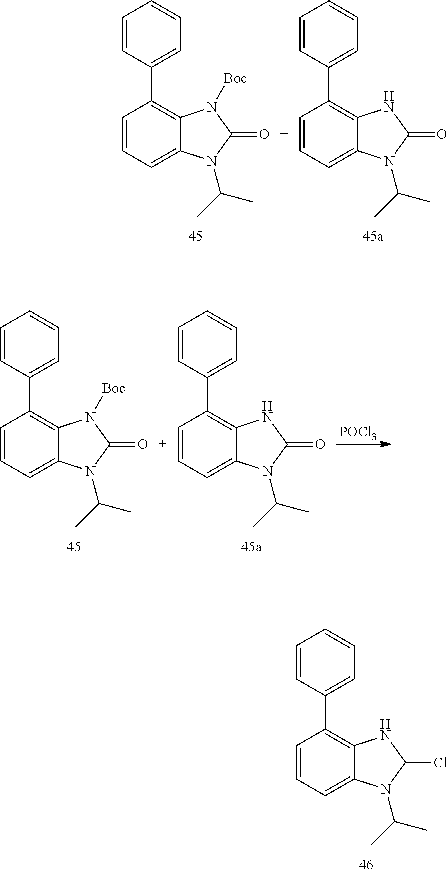

To a solution of compound 37 (20 g, 0.116 mol) in AcOH (65 mL) was added Ac₂O (22 mL) slowly at 10° C., after that HNO₃ was added dropwise at the same temperature, then the mixture was warmed to room temperature and stirred overnight, the reaction mixture was poured into ice water, extracted with EtOAc, the organic layers were combined, washed by brine, dried over anhydrous Na₂SO₄, then the solvent was removed under reduced pressure, the crude was recrystallized with dichloromethane-cyclohexane to afford compound 38 (5.5 g, 18.3%).

To a solution of compound 38 (5.5 g, 21.2 mmol) in ethanol (50 mL) was added conc. HCl (30 mL), the resulting mixture was heated to reflux overnight. The reaction was monitored by TLC. After completion of the reaction, the mixture was cooled by ice water, basified by NH₃.H₂O, extracted with EtOAc, the organic layers were combined, washed by brine, dried over anhydrous Na₂SO₄, then the solvent was removed under reduced pressure, the crude product 39 (4.2 g, 91.3%) was used directly in the next step. ¹H NMR (400 MHz, CDCl₃) δ 8.21 (d, J=2.4 Hz, 1H), 7.73 (d, J=2.4 Hz, 1H), 7.83 (s, 1H).

To a solution of compound 39 (1.5 g) in methanol (15 mL) was added iron powder (1.17 g, 20.9 mmol) and AcOH (376 mg, 6.27 mmol) at 0° C., then the mixture was warmed to room temperature ant stirred overnight, the reaction was monitored by TLC. After completion of the reaction, the solid was filtered off, the filtrate was cooled by ice water, basified by NH₃.H₂O, extracted with EtOAc, the organic layers were combined, washed by brine, dried over anhydrous Na₂SO₄, the solvent was removed under reduced pressure. Purification by flash column chromatography to give compound 40 as brown solid (0.7 g, 54%).

To a solution of compound 40 (10 g, 53.5 mmol) in anhydrous THF (100 mL) was added CDI (17.5 g, 107 mmol), the resulting mixture was stirred at room temperature overnight. The reaction was monitored by TLC. After completion of the reaction, the solvent was removed under reduced pressure, the residue was neutralized with aq. HCl (2M). The solid was filtered and collected, it was dried over vacuum to afford compound 41 (6.1 g, 54.4%). ¹H NMR (400 MHz, DMSO-d₆) δ 11.03 (s, 1H), 10.90 (s, 1H), 7.09 (d, J=8 Hz, 1H), 6.84-6.93 (m, 2H).

To a solution of compound 41 (100 mg, 0.47 mmol) in anhydrous THF (2 mL) was added Boc₂O (409.8 mg, 1.88 mmol), then DMAP (57 mg, 0.47 mmol) was added. The reaction mixture was stirred at room temperature overnight. The reaction was monitored by TLC. After completion of the reaction, the solvent was removed under reduced pressure, the residue was purified by column chromatography (PE:EA=3:1) to afford compound 42 (170 mg, 87.6%).

To a solution of compound 42 (65 mg, 0.16 mmol) in anhydrous THF (2 mL) was added isopropyl amine (18.6 mg, 0.32 mmol), the resulting mixture was stirred at room temperature for 3 hrs. TLC showed completion of reaction, the solvent was removed under reduced pressure, the crude product 43 was used directly in the next step. ¹H NMR (400 MHz, CDCl₃) δ 9.02 (s, 1H), 7.27-7.13 (m, 1H), 6.98-6.92 (m, 2H), 1.60 (s, 9H).

To a solution of compound 43 (50.0 mg, 0.16 mmol) in anhydrous DMF (1.5 mL) were added K₂CO₃ (44 mg, 0.32 mmol) and 2-Iodopropane (54 mg, 0.32 mmol), the reaction was stirred at room temperature overnight, the reaction was monitored with TLC. After completion of the reaction, the reaction mixture was diluted with water (10 mL), neutralized with aq. HCl (2 M), extracted with EtOAc (15 mL×3), the organic layers were combined, washed by brine, dried over anhydrous Na₂SO₄, then the solvent was removed under reduced pressure, the crude was purified with prep-TLC to afford compound 44 (25 mg, 44%). ¹H NMR (400 MHz, CDCl₃) δ 7.19-7.17 (m, 1H), 6.99-6.92 (m, 2H), 4.62-4.54 (m, 1H), 1.61 (s, 9H), 1.46 (d, J=8.0 Hz, 6H).

A flask were charged with compound 44 (110 mg, 0.31 mmol), Na₂CO₃ (65.7 mg, 0.62 mmol), phenylboronic acid (75.8 mg, 0.62 mmol) and Pd(PPh₃)₄ (71.6 mg, 0.062 mmol), the flask was degassed with nitrogen for three times, then 1,4-dioxane (2 mL) and a drop of water were added, the resulting mixture was heated to reflux overnight under nitrogen protection. After completion of the reaction, the mixture was cooled to r.t. and diluted with EtOAc (20 mL), the solid was filtered, the filtrate was concentrated in vacuo. The resulting residue was purified by prep-TLC to give a mixture of compound 45 and 45a (105 mg, 73%).

A flask was charged with compound 45 and 45a (105 mg) then 3 mL of POCl₃ was added and the resulting mixture was heated to reflux overnight. After completion of the reaction, the solvent was removed, the crude product was dissolved in EtOAc (50 mL), basified with aqueous NH₃.H₂O. The organic layer was separated, dried over anhydrous Na₂SO₄, the solvent was removed under reduced pressure to give compound 46 (50 mg, 61.7%). Compound 46 was used in the synthesis of compound 203.

### 2.4 Synthesis of Precursor Compound 51

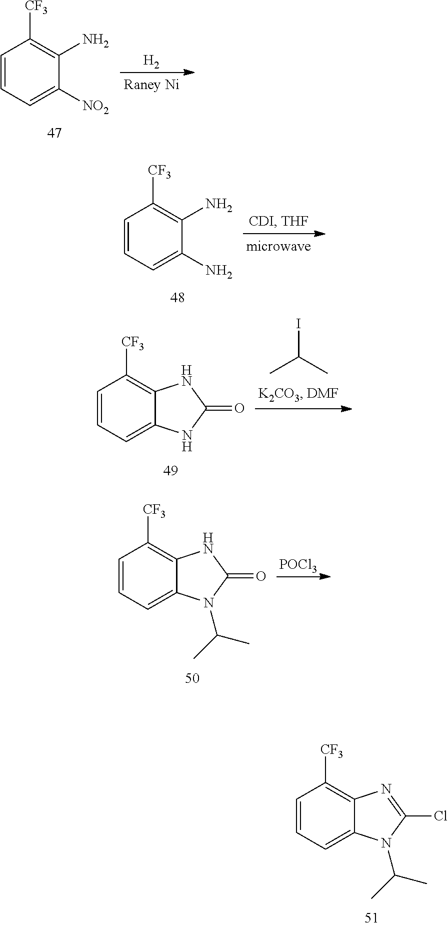

To a solution of compound 47 (780 mg, 3.78 mmol) in MeOH (20 mL) was added Raney Ni (0.5 g), the reaction mixture was hydrogenated at a pressure of 50 psi for 6 h. TLC indicated the reaction was complete. The catalyst was filtered off and the filtrate was evaporated in vacuo to give compound 48 as a brown solid (580 mg, 87%).

A microwave tube was charged with compound 48 (500 mg, 2.84 mmol), CDI (1.85 g, 11.36 mmol) and anhydrous TRF (20 mL), the reaction mixture was heated at 120° C. under microwave for 20 min. After cooling to r.t, the mixture was concentrated, the residue was purified with column chromatography (PE:EA=1:1) to afford compound 49 (300 mg, 52%).

To a solution of 49 (150 mg, 1.08 mmol) in 5 mL of DMF was added K₂CO₃ (200 mg, 1.48 mmol) and 2-Iodopropane (100 mg, 0.59 mmol). The mixture was stirred at room temperature for 24 hrs. LCMS monitored the reaction. Then the mixture was diluted with 20 mL of water, extracted with EtOAc (20 mL×3). The combined organic layers were washed with brine, dried over Na₂SO₄, filtered and concentrated. The resulting residue was purified with prep-TLC to afford compound 50 (20 mg, 11%).

A mixture of 50 (30 mg, 0.12 mmol) in 5 mL of POCl₃ was heated to reflux for 4 hrs. TLC analysis showed the reaction completed. The mixture was poured into ice water, neutralized with saturated aqueous NaHCO₃, and then extracted with ethyl acetate (15 mL×3), the organic layers were combined, washed by brine, dried over anhydrous sodium sulfate, solvent was removed under reduced pressure to gave compound 51 (32 mg, 100%). The crude compound 51 was used directly in the synthesis of compound 204.

### 2.5 Synthesis of Precursor Compound 56

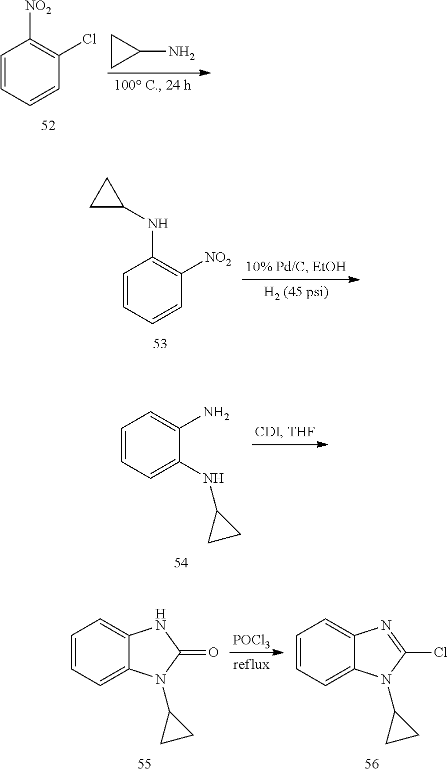

A mixture of 2-chloronitrobenzene 52 (3.14 g, 20 mmol) and cyclopropyl-amine (3.5 mL, 50 mmol) was placed in a high-pressure vessel and heated at 100° C. for 24 h. Then the reactor was opened, the reaction mixture was diluted with water and extracted with CH₂Cl₂, and the extract was washed with water and dried over Na₂SO₄. The solvent was evaporated in vacuo, and the residue was purified by flash chromatography to yield compound 53 (2.55 g, 71.6%) as an orange oil.

A solution of compound 53 (2.55 g, 14.3 mmol) in EtOH (100 mL) was hydrogenated over 10% palladium/carbon (0.6 g) at 45 psi for 4 h. The catalyst was filtered off and the filtrate was evaporated in vacuo to give compound 3 (1.8 g, 85.1%).

A solution of compound 54 (500 mg, 3.38 mmol) and N,N-carbonyldiimidazole (550 mg, 3.38 mmol) in dry TRF (10 mL) was stirred at room temperature for 20 hrs and then evaporated. The residue was taken up in water and extracted with CH₂Cl₂. The dried organic phase was evaporated, and the residue was purified by flash chromatography to give compound 55 as a brown solid (500 mg, 85.0).

Compound 55 (250 mg, 1.44 mmol) was heated in a 30 mL of high-pressure vessel with POCl₃ (4 mL) and HCl (2 drops) at 150° C. for 3 h. The reaction mixture was poured into ice-water, neutralized with 50% NaOH, and extracted with CH₂Cl₂. The extract was washed with water, dried over Na₂SO₄, and concentrated to yield compound 56 (260 mg, 94%) as a brown solid. It is used for making compound 205.

### 2.6 Synthesis of Precursor Compound 61

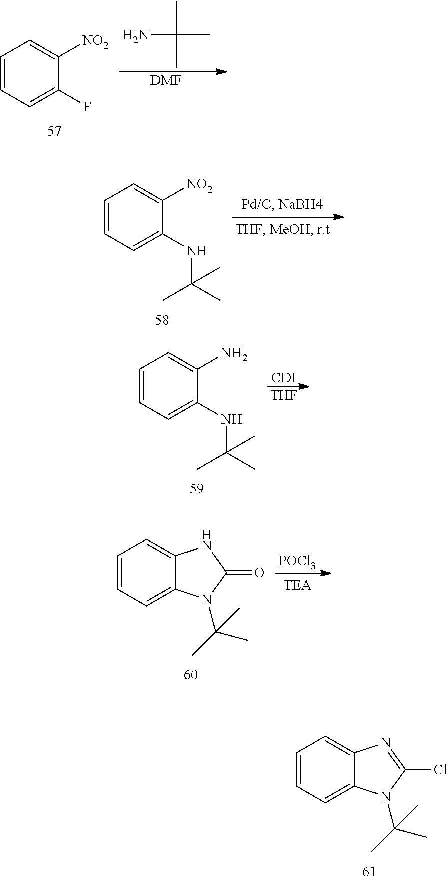

To a solution of 2-fluoronitrobenzene 57 (2.8 g, 20 mmol) in DMF was added t-butyl amine (4.38 mL, 60 mmol). The mixture was stirred at room temperature over night, the reaction was detected by TLC after completion of the reaction, the mixture was diluted with water and extracted with EtOAc, and the extract was washed with brine and dried over Na₂SO₄. The solvent was evaporated in vacuum, and the residue was purified by flash chromatography to yield compound 58 (3.5 g, 90%).

To a suspension of compound 58 (900 mg, 4.6 mmol),360 mg 5% palladium on carbon and 360 mg sodium borohydride in anhydrous TRF (15 mL) was added 7.5 mL of methanol dropwise. The reaction was detected by TLC. After completion of the reaction, the catalyst was filtered off and the filtrate was poured into saturated aqueous solution of ammonium chloride, extracted with ethyl acetate, the organic layer was separated, dried over anhydrous Na₂SO₄, concentrated in vacuum to give compound 59 (754 mg, 100%).

A solution of compound 59 (754 mg, 4.6 mmol) and N,N-carbonyldiimidazole (1.9 g, 11.5 mmol) in dry TRF (10 mL) was stirred at room temperature for 20 h and then evaporated. The residue was taken up in water and extracted with EtOAc. The dried organic phase was evaporated, and the residue was purified by flash chromatography to give compound 60 (600 mg, 68.6%).

To a flask (10 mL) were added compound 60 (95 mg, 0.5 mmol) and Et₃N (50.5 mg, 0.5 mmol), then 3 mL of POCl₃ was added and the resulting mixture was heated to reflux overnight. After completion of the reaction, the solvent was removed, the crude product was dissolved in EtOAc, basified with aqueous NaHCO₃, the organic layer was separated, dried over anhydrous Na₂SO₄, then the solvent was removed to give crude compound 61 (90%). Compound 61 was use for the preparation of compound 206.

### 2.7 Synthesis of Precursor Compound 65

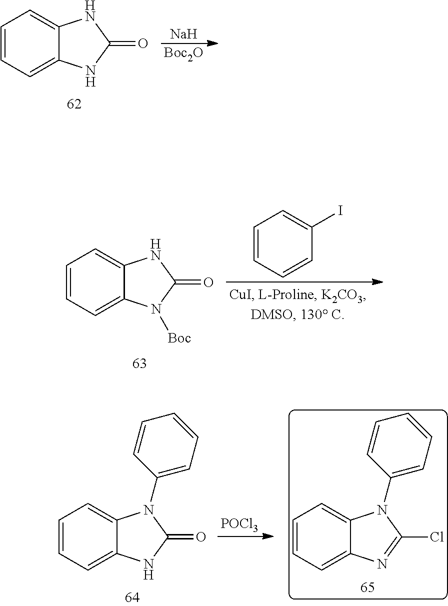

NaH (60% in mineral oil, 228 mg, 5.7 mmol) was added portionwise to a stirred solution of compound 62 (0.7 g, 5.2 mmol) in dry DMF (8 mL) maintained under an atmosphere of N₂. After 75 min, di-tert-butyl dicarbonate (1.1 g, 5.2 mmol) was added dropwise and the mixture stirred at room temperature overnight. Both TLC and LCMS showed the reaction was complete. The resulting mixture was poured into ice cold saturated NH₄Cl solution and isolated solid, filtered, dried to give crude product 63 (1.0 g, 83.3%).

A schlenk tube was charged with compound 63 (1 eq.), CuI (0.2 eq.), trans-4-hydroxy-L-proline (0.4 eq.) and K₃CO₃ (2.0 eq.), evacuated and backfilled with nitrogen. Iodobenzene (1.0 eq.) and DMSO were added successively. The reaction mixture was stirred at 130° C. overnight. After cooling to r.t, the reaction mixture was poured into saturated NH₄Cl solution. The mixture was extracted with ethyl acetate. The organic layer was dried over Na₂SO₄, concentrated and purified by column chromatography on silica gel to afford compound 64 (Yield 37.9%). ¹H NMR (300 MHz, DMSO-d₆) δ 7.65 (m, 4H), 7.50 (m, 1H), 7.00˜7.20 (m, 4H).

A mixture of compound 64 in POCl₃ was refluxed for 6 h. Most of the POCl₃ was removed in vacuo and the residue was quenched with ice water and basified with aq. NaHCO₃ to pH=7-8. The mixture was extracted with ethyl acetate. The organic layer was dried over Na₂SO₄ and evaporated to afford crude product 65 (Yield, 92%). Compound 65 was use for the preparation of compound 207.

### 2.8 Synthesis of Precursor Compound 71

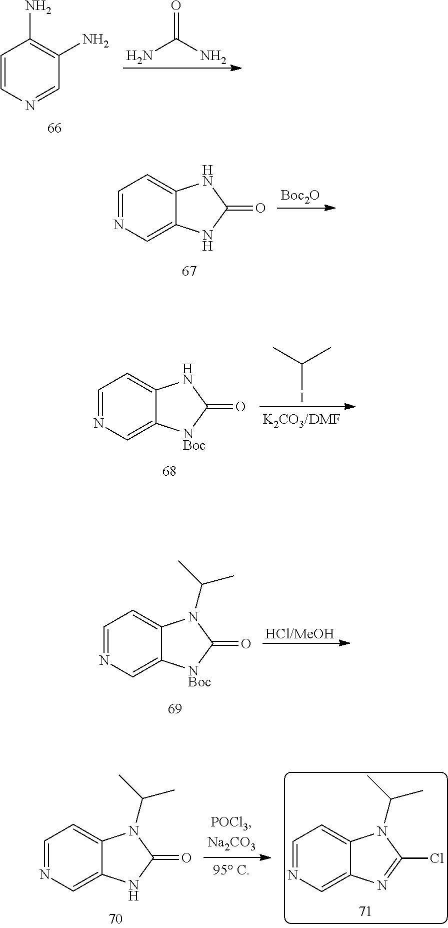

A mixture of 66 (5 g, 45.9 mmol) and urea (16.5 g, 275 mmol) was heated at 165° C. for 4 h. After cooling to r.t, water (300 mL) was added, the mixture was heated to reflux until the solid was dissolved. Then the mixture was cooled to r.t., and placed for 28 hrs. Filtered and collected the solid to afford compound 67 (4.1 g, 66%).

To a solution of 67 (3 g, 22.2 mmol) in DMF (30 mL) was added NaH (60%, 924 mg, 23.1 mmol) in portions at 0° C. After stirring for 30 min, Boc₂O (5.28 g, 24.2 mmol) was added. The mixture was stirred overnight at r.t. After completion of the reaction, DMF was removed in vacuo., the residue was dissolved in EtOAc (100 mL), PE was added, precipitate formed, filtered and got compound 68 (1.8 g, 34.5%).

To a solution of 68 (1.8 g, 7.7 mmol) in DMF (18 mL) was added K₂CO₃ (2.11 g, 15.3 mmol) and 2-iodopropane (2.5 g, 14.6 mmol). The mixture was stirred at r.t. TLC monitored the reaction. The reaction mixture was poured into saturated NH₄Cl solution. The mixture was extracted with ethyl acetate. The organic layer was dried over Na₂SO₄, concentrated and purified by column chromatography on silica gel to afford compound 69 (500 mg, 24%).

The solution of compound 69 (0.53 g, 1.9 mmol) in 6 mL of HCl/MeOH was stirred for 16 h at room temperature. TLC analysis showed the reaction completed. All the volatiles were removed under reduced pressure. The residue was neutralized with NH₃.H₂O, extracted with ethyl acetate (50 mL×3). The organic layers were combined, washed by brine, dried over anhydrous sodium sulfate, the solvent was removed under reduced pressure, and crude compound 70 was used directly in the next step (0.33 g, 97%).

To the solution of compound 70 (200 mg, 1.13 mmol) in 3 mL of POCl₃ was added Na₂CO₃ (120 mg, 1.13 mmol). The reaction mixture was heated at reflux for 16 h. TLC analysis showed the reaction completed. The mixture was poured into ice water, neutralized with saturated aqueous NaHCO₃, and then extracted with ethyl acetate (20 mL×3), the organic layers were combined, washed by brine, dried over anhydrous sodium sulfate, solvent was removed under reduced pressure to give compound 71 (80 mg, 36.2%). The crude compound 71 was used directly in the synthesis of compound 209.

### 2.9 Synthesis of Macrocylic Precursors

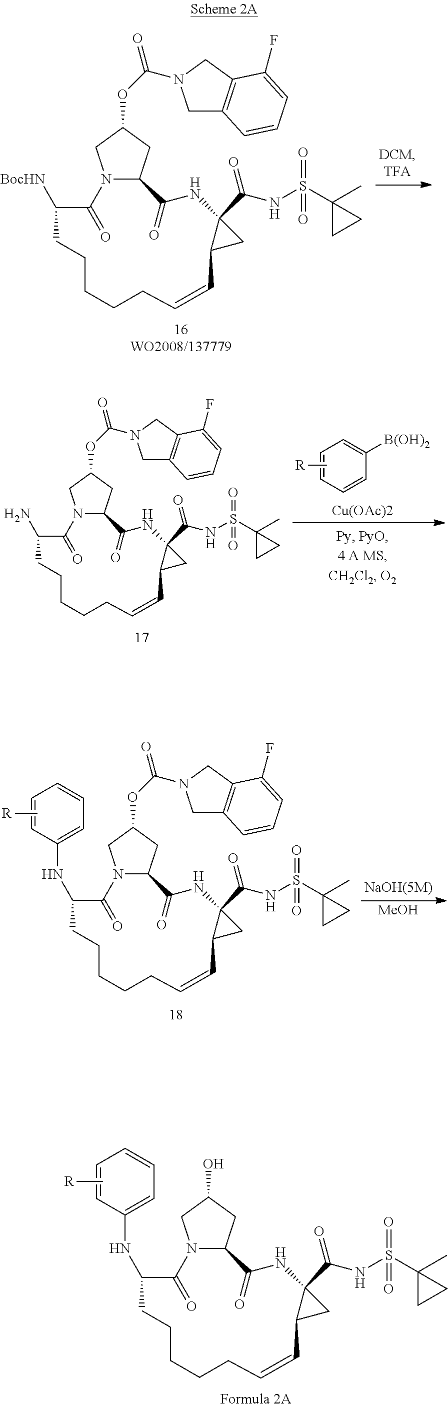

The isoindoline carbamate 16 can be synthesized according to WO 2008/137779. Compound 16 can be treated with acid, for example TFA in DCM, to remove the Boc protecting group thereby providing compound 17. Compound 17 can be treated with optionally substituted aryl boronic acids under Cu²⁺-catalyzed conditions thereby providing isoindoline carbamates having general structure 18. The isoindoline carbamate having general structure 18 can be treated under basic conditions, for example aqueous sodium hydroxide in methanol, to hydrolyse the isoindoline carbamate thereby providing alcohols having general structure Formula 2A.

### 2.10 Synthesis of Compound 201

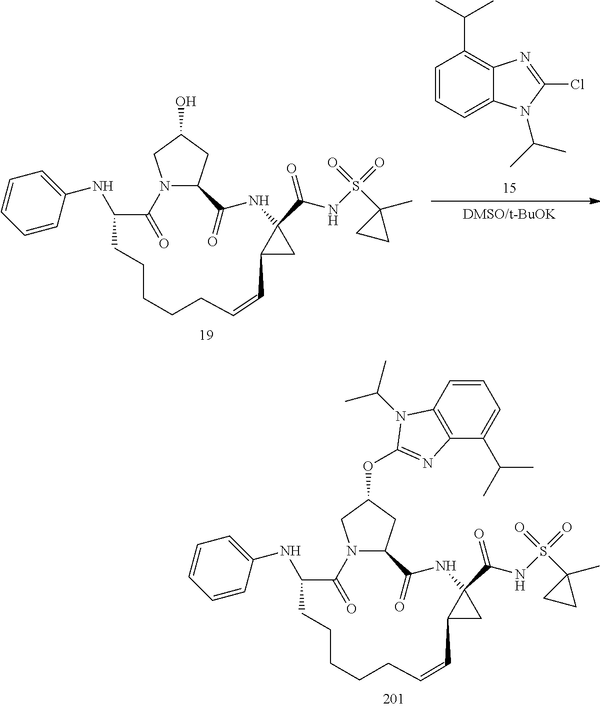

Compound 19 was synthesized according to Scheme 2A. To a solution of compound 19 (150 mg, 0.27 mmol) in 2 mL of DMSO was added t-BuOK (151 mg, 1.35 mmol) in portions at ambient temperature, then the mixture was stirred for 2 h at ambient temperature. After that, compound 7 (76 mg, 0.32 mmol) was added, the resulting mixture was stirred at ambient temperature for 12 h, the reaction was monitored by LCMS. After completion of the reaction, the mixture was cooled by ice water, acidified by aq. HCl (2 M) to pH=5-6, then the mixture was extracted with ethyl acetate (20 mL×3), the organic layers were combined, washed by brine, dried over anhydrous sodium sulfate, solvent was removed under reduced pressure, the crude product was purified by prep-HPLC to afford compound 201. 77.3 mg, 36.8%. MS (ESI) m/z (M+H)⁺ 759.

### 2.11 Synthesis of Compound 202

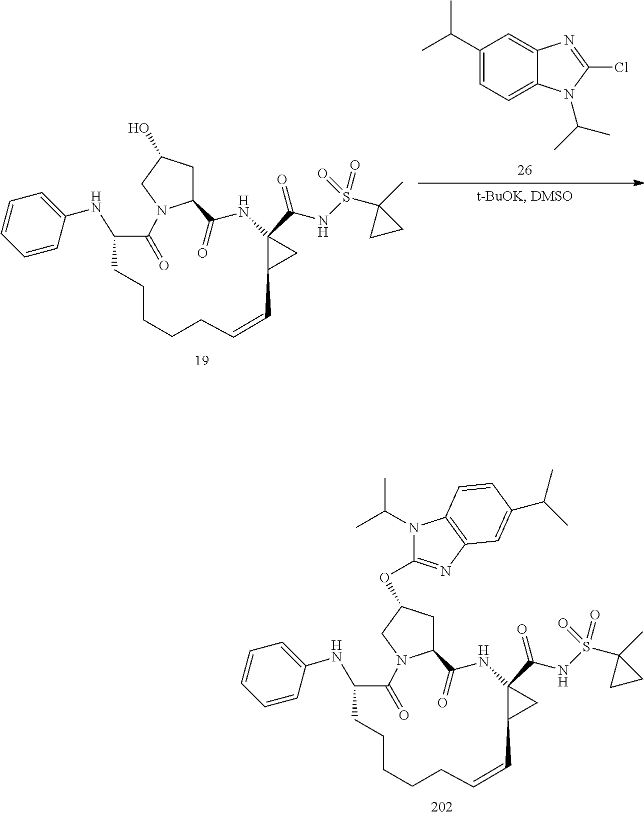

Compound 202 was prepared using the similar to that of compound 201. 56.3 mg, 17.1%. MS (ESI) m/z (M+H)⁺ 758.9.

### 2.12 Synthesis of Compound 203

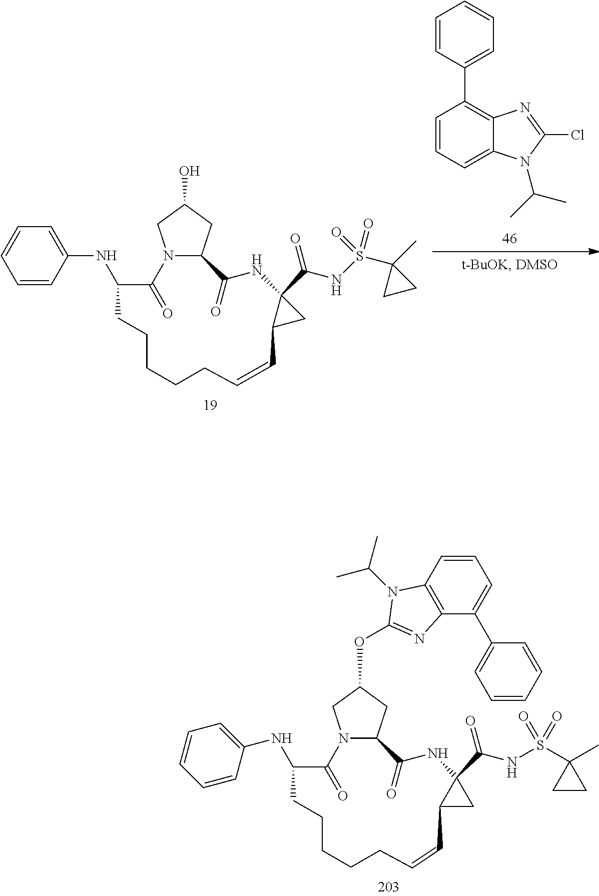

To a solution of compound 19 (310 mg, 0.55 mmol) in 4 mL of DMSO was added t-BuOK (215 mg, 1.92 mmol.). The resulting mixture was stirred at r.t. for 1.5 hour before the addition of compound 46 (150 mg, 0.55 mmol.). The reaction mixture was stirred at r.t. overnight. After the reaction completion, the reaction was quenched with water (10 mL), aq. HCl (2 M) was added to acidify the mixture to pH=6, then the mixture was extracted by EtOAc (30 mL×3), the organic layers were combined, washed by brine, dried over anhydrous Na₂SO₄, concentrated in vacuo. The residue was purified with prep-HPLC to give compound 203 (97 mg, 22.5%). MS (ESI) m/z (M+H)⁺ 793.2.

### 2.13 Synthesis of Compound 204

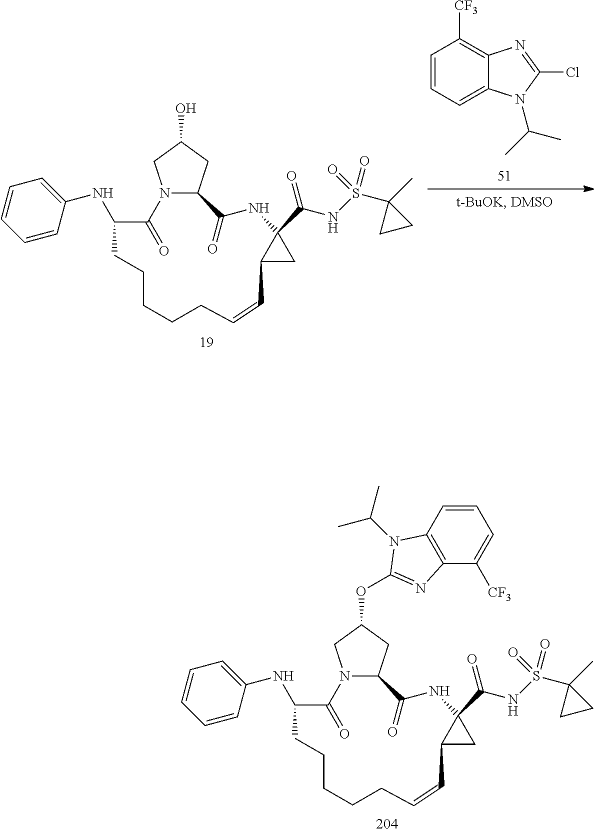

Compound 204 was prepared using a procedure similar to that of compound 203. 16 mg, 12%. MS (ESI) m/z (M+H)⁺ 785.3.

### 2.14 Synthesis of Compound 205

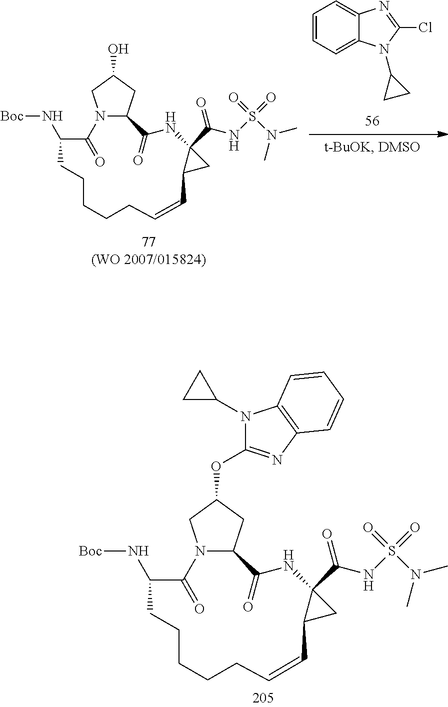

Compound 77 was prepared using methods described in PCT Publication No. WO 2007/015824, which is incorporated herein by reference in its entirety. To a solution of compound 77 (1 eq) in 4 mL of DMSO was added t-BuOK (5 eq.). The resulting mixture was stirred at room temperature for 1.5 h before the addition of compound 56 (1.5 eq.), and it was stirred overnight. The reaction was quenched with water (10 mL), extracted with ethyl acetate, washed with brine, dried over Na₂SO₄, concentrated to get a residue, which was purified by prep-HPLC to give target compound. 71.3 mg, 28.0%. MS (ESI) m/z (M+H)⁺ 728.1.

### 2.15 Synthesis of Compound 206

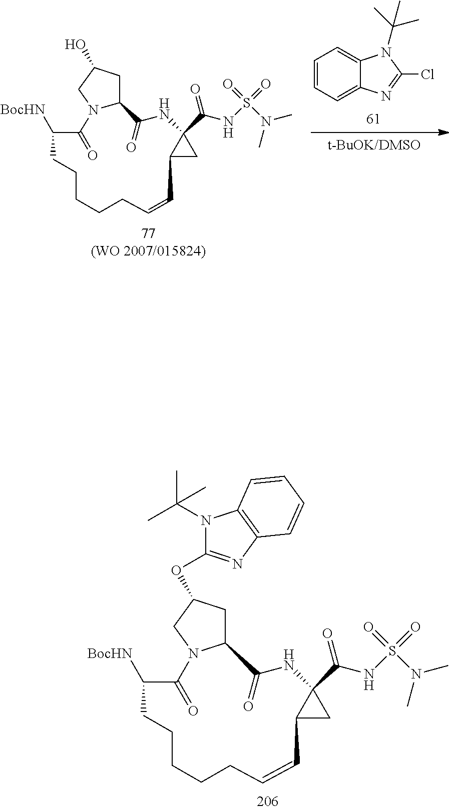

To a solution of compound 77 (190 mg, 0.33 mmol) in 4 mL of DMSO was added t-BuOK (184.8 mg, 1.65 mmol.). The resulting mixture was stirred at room temperature for 1.5 h before the addition of compound 61 (76 mg, 0.37 mmol.), and it was stirred overnight. The reaction was quenched with water (10 mL), 2 M HCl was added to acidify the mixture to pH=6, then the mixture was extracted by EtOAc, the organic layers were combined, washed by brine, dried over anhydrous Na₂SO₄, the crude was purified with prep-HPLC to give compound 206 (51 mg, 20.7%). MS (ESI) m/z (M+H)⁺ 744.

### 2.16 Synthesis of Compound 207

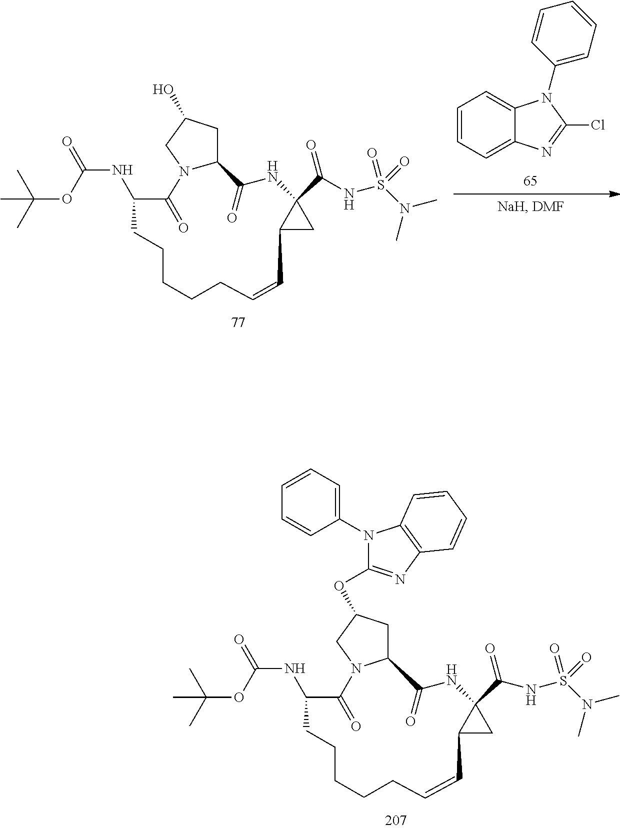

To a solution of compound 77 (1 eq.) in DMF was added NaH (6 eq) at 0° C. The reaction mixture was stirred at 0° C. for 1 h under N₂. To the resulting solution was added compound 65 (1.2 eq.) at 0-5° C. The reaction mixture was stirred at room temperature overnight under N₂. To the reaction mixture was added water. The mixture was extracted with ethyl acetate and dried over Na₂SO₄. The solvent was removed to give crude mixture, it was purified by prep-HPLC to give compound 207. 67.2 mg, 26.7%. MS (ESI) m/z (M+H)⁺764.2.

### 2.17 Synthesis of Compound 208

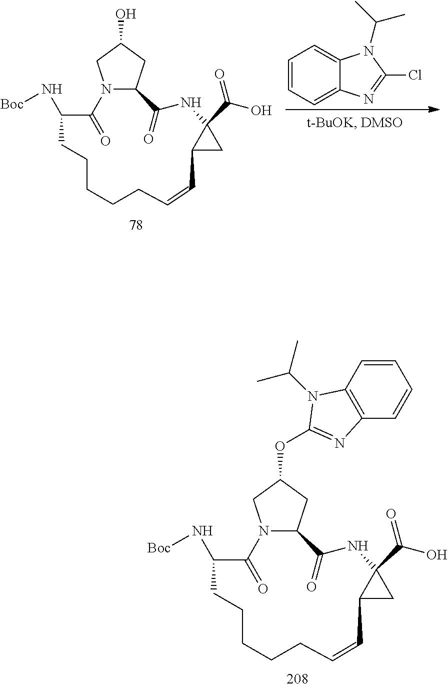

Compound 78 was prepared according to PCT Publication No. WO 2007/015824, which is incorporated herein by reference in its entirety. Compound 208 was prepared following the procedure similar to that of compound 206. 11 mg, 18%. MS (ESI) m/z (M+H)⁺ 624.2

### 2.18 Synthesis of Compound 209

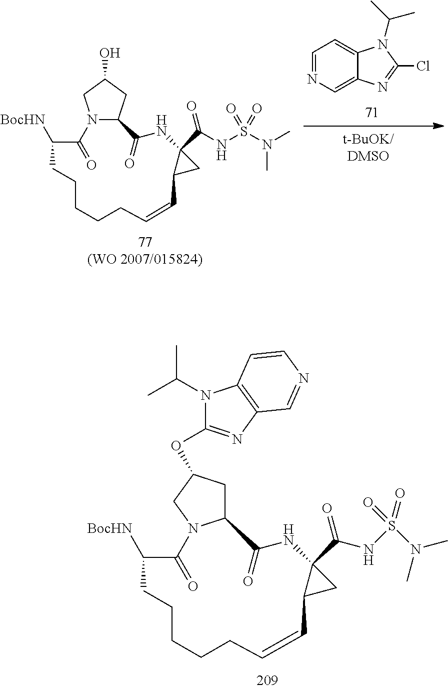

To a solution of compound 77 (1 eq.) in 2 mL of DMSO was added t-BuOK (5 eq) in portions at ambient temperature, then the mixture was stirred for 2 h at ambient temperature. After that, compound 71 (1.2 eq) was added, the resulting mixture was stirred at ambient temperature for 12 h, the reaction was monitored by LCMS. After completion of the reaction, the mixture was cooled by ice water, acidified by aq. HCl (2 M) to pH=8, then the mixture was extracted by ethyl acetate (20 mL×3), the organic layers were combined, washed by brine, dried over anhydrous sodium sulfate, solvent was removed under reduced pressure, the crude product was purified by HPLC to afford compound 209. 38 mg, 10.4%. MS (ESI) m/z (M+Na)⁺ 731.

### 2.19 Synthesis of Compound 210

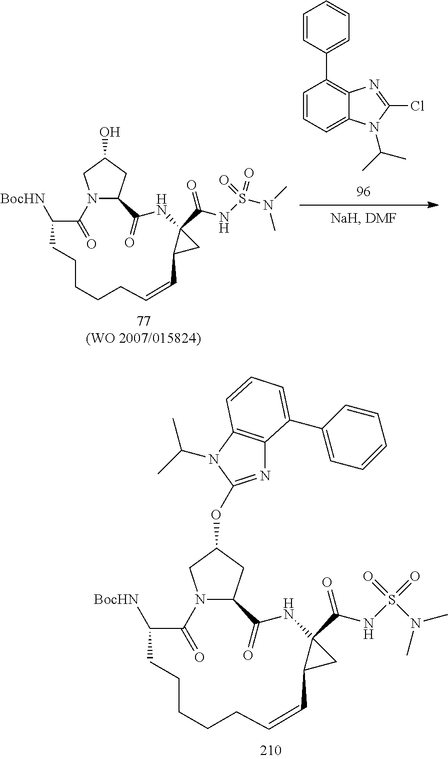

Compound 77 was prepared according to PCT Publication No. WO 2007/015824, which is incorporated herein by reference in its entirety. Compound 210 was prepared following the procedure similar to that of compound 209. 6.3 mg, 9%. MS (ESI) m/z (M+H)⁺ 806.3.

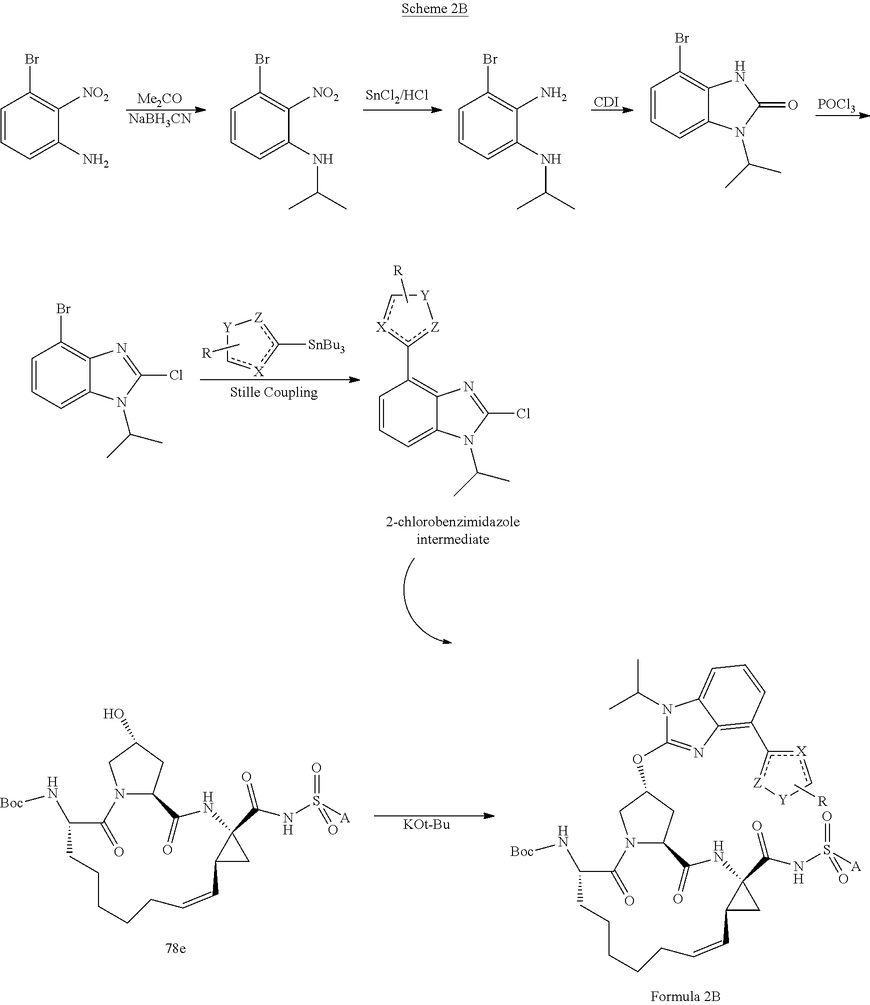

### 2.20 Synthesis of 2-chlorobenzimidazol intermediates

### Stage 1-1. 3-bromo-N-isopropyl-2-nitroaniline

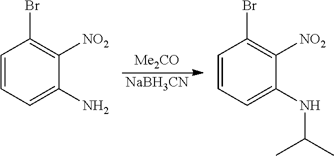

To a solution of 3-bromo-2-nitroaniline (5.425 g, 25 mmol) in methanol (80 ml) were added acetone (3.67 ml, 50 mmol) and conc. HCl (2.7 mL) and the mixture was stirred for one hour at room temperature. Solution of sodium cyanoborohydride (2.36 g, 37.5 mmol) in methanol (20 mL) was added portion-wise at 0° C. and the mixture was stirred for 2 hours at room temperature. Reaction mixture was made basic (pH 9) and most of the solvent was removed under reduced pressure. The residue was taken into DCM-water, organic phase was separated, washed with water, dried over sodium sulfate and the solvent was removed under vacuum. The tile compound was isolated as an oil by column chromatography in 5 to 20% ethyl acetate-hexane. Yield 5.24 g (80.9%). ¹H-NMR (CDCl₃), δ: 7.12 (dd, 1H), 6.90 (dd, 1H), 6.75 (dd, 1H), 5.54 (br. s, 1H), 3.70 (m, 1H), 1.25 (d, 6H).

### Stage 1-2. 3-bromo-N-1-isopropylbenzene-1,2-diamine

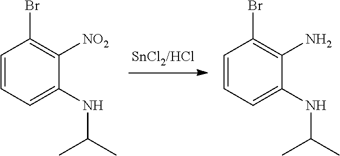

To a solution of the nitro aniline (5.24 g, 20.2 mmol) in methanol (50 mL) was added tin (II) chloride dehydrate (13.7 g, 60.6 mmol) followed by aqueous conc. HCl (8 mL). The reaction was refluxed for 6 h and then cooled down to room temperature. Celite (˜10 g) was added and the reaction was carefully neutralized by addition of ammonium hydroxide (30 ml) under cooling. Solids were filtered off and washed with DCM. Organic layer was separated, washed with water, dried over sodium sulfate and evaporated under vacuum. The bis-amino compound was isolated as pale-yellow solid by column chromatography in 20-50% ethyl acetate-hexane. Yield: 4.29 g (92.8%). ¹H-NMR (CDCl₃), δ: 6.91 (dd, 1H), 6.66 (dd, 1H), 6.60 (dd, 1H), 3.74 (br. s, 2H), 3.58 (m, 1H), 3.20 (br. s, 1H), 1.23 (d, 6H).

### Stage 1-3. 4-bromo-1-isopropyl-1H-benzo[d]imidazol-2 (3H)-one

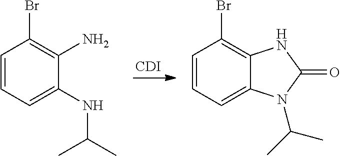

To a solution of the bis-amino compound (4.29 g, 18.7 mmol) in TRF (30 mL) was added carbonyldiimidazole (4.55 g, 28 mmol) and the reaction was refluxed for 10 h. Aqueous 2N HCl (30 ml) was added and the mixture was extracted with ethyl acetate. Organic phase was washed with brine, dried over magnesium sulfate and evaporated to afford 4.59 g (96%) of off-white solid which was used on next step without any further purification. ¹H-NMR (CDCl₃), δ: 8.47 (br. s, 1H), 7.17 (dd, 1H), 7.07 (dd, 1H), 6.95 (dd, 1H), 4.71 (m, 1H), 1.54 (d, 6H).

### Stage 1-4. 4-bromo-2-chloro-1-isopropyl-1H-benzo[d]imidazole

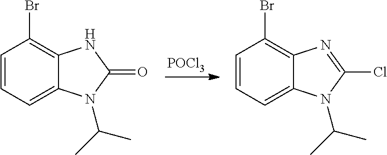

To 2-hydroxybenzimidazole from previous step (4.59 g, 18 mmol) was added phosphorus(V) oxychloride (5 ml) and the mixture was refluxed overnight. After it was cooled to 0° C., the reaction was quenched by careful addition of ice and neutralized by aqueous ammonium hydroxide (˜25 mL). The product was extracted by DCM; organic phase was dried over sodium sulfate and evaporated. Column chromatography (10 to 20% ethyl acetate-hexane) afforded the title compound as white solid. Yield: 4.85 g (98.6%). ¹H-NMR (CDCl₃), δ: 7.46 (dd, 1H), 7.44 (dd, 1H), 7.13 (dd, 1H), 4.92 (m, 1H), 1.66 (d, 6H).

### Stage 1-5. 2-(2-chloro-1-isopropyl-1H-benzo[d]imidazol-4-yl)thiazole

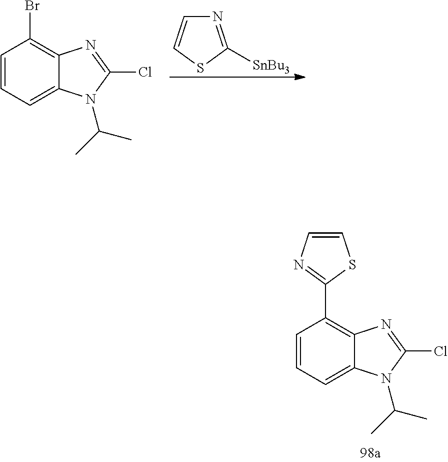

In a vial a solution of arylbromide (121 mg, 0.44 mmol) and tributyltin thiazole (166 mg, 0.44 mmol) in toluene (3 mL) was degassed by bubbling argon for 20 min. Pd[P(Ph)₃]₄ (23 mg, 0.02 mmol) was added, the vial was sealed and heated using microwave apparatus for 3 h at 155° C. The reaction mixture was filtered trough a silica gel pad, evaporated and separated by column chromatography in 15 to 30% ethyl acetate-hexane. Yield: 85 mg (69.6%). White solid. ¹H-NMR (CDCl₃), δ: 8.21 (dd, 1H), 7.96 (d, 1H), 7.55 (dd, 1H), 7.48 (d, 1H), 7.36 (dd, 1H), 4.97 (m, 1H), 1.69 (d, 6H).

The following 2-chlorobenzimidazol intermediates were synthesized following the procedure described above.

TABLE 2
2-chlorobenzimidazol intermediates prepared.

| Intermediate | Structure | Yield |
|---|---|---|
| 98b | 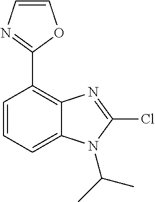 | Yield: 53%. ¹H-NMR (CDCl₃), δ: 7.99 (dd, 1H), 7.85 (d, 1H), 7.59 (dd, 1H), 7.36 (d, 1H), 7.34 (dd, 1H), 4.97 (m, 1H), 1.67 (d, 6H). |
| 98c | 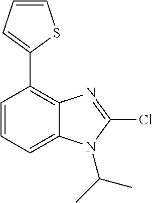 | Yield: 83%. ¹H-NMR (CDCl₃), δ: 8.01 (dd, 1H), 7.54 (d, 1H), 7.40 (d, 1H), 7.35 (dd, 1H), 7.26 (dd, 1H), 7.15 (dd, 1H). |
| 98d | 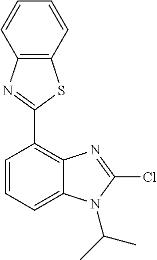 | Yield: 57%. ¹H-NMR (CDCl₃), δ: 8.39 (dd, 1H), 8.13 (d, 1H), 7.97 (d, 1H), 7.62 (dd, 1H), 7.50 (ddd, 1H), 7.41 (dd, 1H), 7.39 (dd, 1H), 5.0 (m, 1H), 1.71 (d, 6H). |
| 98e | 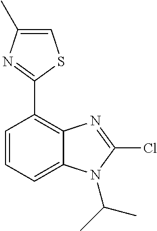 | Yield: 72%. ¹H-NMR (CDCl₃), δ: 8.19 (dd, 1H), 7.53 (dd, 1H), 7.34 (dd, 1H), 7.04 (q, 1H), 4.96 (m, 1H), 2.56 (d, 3H), 1.69 (d, 6H). |
| 98f | 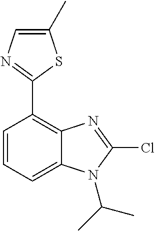 | Yield: 75%. ¹H-NMR (CDCl₃), δ: 8.14 (dd, 1H), 7.59 (q, 1H, 7.51 (dd, 1H), 7.34 (dd, 1H), 4.97 (m, 1H), 2.55 (d, 3H), 1.69 (d, 6H). |
| 98g | 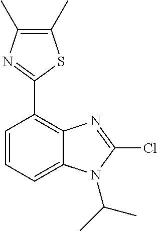 | Yield: 58%. ¹H-NMR (CDCl₃), δ: 8.13 (dd, 1H), 7.49 (dd, 1H), 7.32 (dd, 1H), 4.95 (m, 1H), 2.43 (s, 6H), 1.68 (d, 6H). |
| 98h | 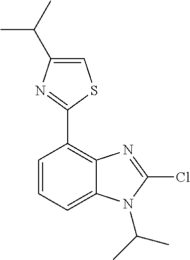 | Yield: 62%. ¹H-NMR (CDCl₃), δ: 8.22 (dd, 1H), 7.51 (dd, 1H), 7.34 (dd, 1H), 7.04 (d, 1H), 4.96 (m, 1H), 3.22 (m, 1H), 1.68 (d, 6H), 1.38 (d, 6H). |
| 98i | 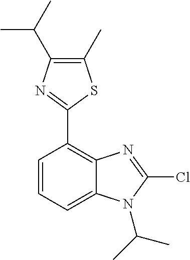 | Yield: 39%. ¹H-NMR (CDCl₃), δ: 8.19 (dd, 1H), 7.47 (dd, 1H), 7.32 (dd, 1H), 4.95 (m, 1H), 3.13 (m, 1H), 2.45 (s, 3H), 1.68 (d, 3H), 1.34 (d, 6H). |
| 98j | 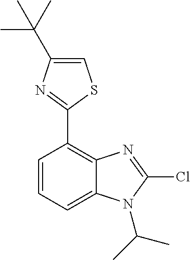 | Yield: 72%. ¹H-NMR (CDCl₃), δ: 8.25 (dd, 1H), 7.50 (dd, 1H), 7.05 (s, 1H), 4.96 (m, 1H), 1.68 (d, 6H), 1.42 (s, 9H). |
| 98k | 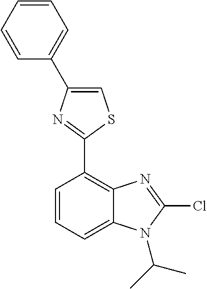 | Yield: 74%. ¹H-NMR (CDCl₃), δ: 8.40 (dd, 1H), 8.05-8.08 (m, 2H), 7.66 (s, 1H), 7.56 (dd, 1H), 7.44-7.48 (m, 2H), 7.39 (dd, 1H), 7.35 (m, 1H). |
| 98l | 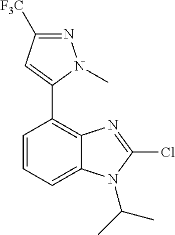 | Yield: 45%. ¹H-NMR (CDCl₃), δ: 7.61 (dd, 1H), 7.35 (dd, 1H), 7.26 (dd, 1H), 6.70 (s, 1H), 4.98 (m, 1H), 3.94 (s, 3H), 1.70 (d, 6H). |
| 98m | 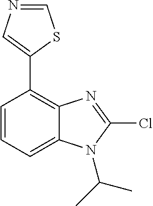 | Yield: 72%. ¹H-NMR (CDCl₃), δ: 8.84 (s, 1H), 8.66 (s, 1H), 7.55 (dd, 1H), 7.47 (dd, 1H), 7.29 (dd, 1H), 4.96 (m, 1H), 1.69 (d, 6H). |
| 98n | 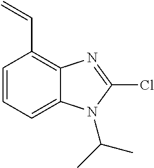 | Yield 65%. ¹H-NMR (CDCl₃), δ: 7.20-7.40 (m, 4H), 6.24 (dd, 1H), 5.48 (dd, 1H), 4.92 (m, 1H), 1.65 (d, 6H). |

### 2.21 Synthesis of compounds 211-237

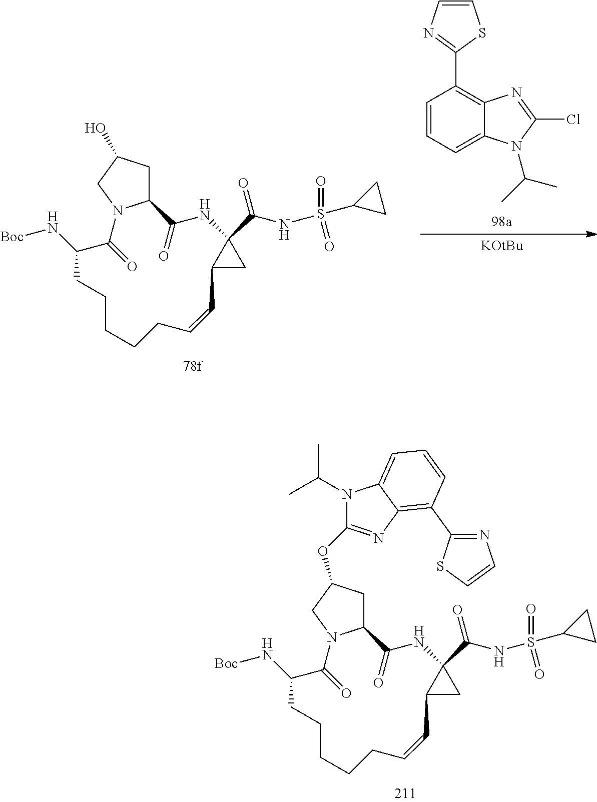

To a solution of hydroxyl macrocyclic intermediate 78f (192 mg, 0.336 mmol) and benzimidazole 98a (85 mg, 0.306 mmol) in anhydrous DMSO (5 mL) was added potassium tert-butylate (151 mg, 1.344 mmol) and the reaction was allowed to proceed for 2 h at room temperature. After addition of water the reaction was neutralized by 2N aqueous hydrochloric acid (0.8 mL) and extracted with ethyl acetate. Organic phase was washed by brine, dried over magnesium sulfate and the solvent was removed under reduced pressure. The residue was purified by column chromatography using ethyl acetate-hexane (from 50 to 100%) as eluent. Yield: 92 mg (37%). White foam. ¹H-NMR (DMSO-d₆), δ: 11.13 (s, 1H), 8.93 (s, 1H), 8.00 (d, 1H), 7.94 (d, 1H), 7.82 (d, 1H), 7.57 (d, 1H), 7.22 (dd, 1H), 7.15 (d, 1H), 5.84 (m, 1H), 5.61 (dt, 1H), 5.12 (dd, 1H), 4.74 (m, 1H), 4.56 (m, 1H), 4.43 (dd, 1H), 3.97-4.07 (m, 2H), 2.69-2.88 (m, 2H), 2.61-2.65 (m, 1H), 2.40-2.46 (m, 1H), 2.30-2.36 (m, 2H), 1.58-1.60 (m, 2H), 1.52 (d, 3H), 1.50 (d, 3H), 1.30-1.44 (m, 5H), 1.28 (s, 9H), 1.18-1.22 (m, 2H), 0.96-1.14 (m, 5H).

The following compounds 212-237 were prepared according to Scheme 2B.

TABLE 3
Compounds prepared according to Scheme 2B.

| Compound | Structure | Yield |
|---|---|---|
| 212 | 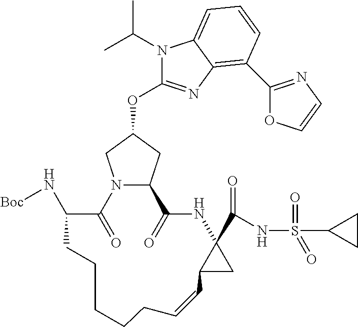 | Yield: 28%. ¹H-NMR (DMSO-d₆), δ: 11.09 (s, 1H), 8.92 (s, 1H), 8.28 (s, 1H), 7.73 (d, 1H), 7.61 (d, 1H), 7.41 (s, 1H). 7.22 (dd, 1H), 7.14 (d, 1H), 5.80 (m, 1H), 5.61 (dt, 1H), 5.12 (dd, 1H), 4.73 (m, 1H), 4.49 (d, 1H), 4.40 (dd, 1H), 3.92 (dd, 2H), 2.89 (m, 1H), 2.58-2.80 (m, 2H), 2.30-2.42 (m, 2H), 1.62-1.80 (m, 2H), 1.56-1.60 (m, 2H), 1.49 (d, 3H), 1.47 (d, 3H), 1.30-1.42 (m, 5H), 1.27 (s, 9H), 0.94-1.22 (m, 7H). |
| 213 | 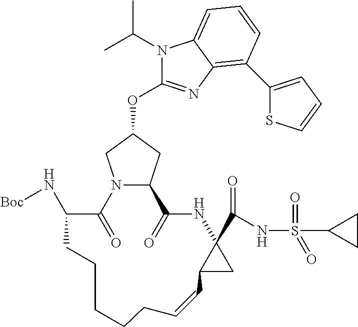 | Yield: 47%. ¹H-NMR (DMSO-d₆), δ: 11.11 (s, 1H), 8.92 (s, 1H), 8.03 (d, 1H), 7.55 (d, 1H), 7.45 (d, 1H), 7.39 (d, 1H), 7.10-7.19 (m, 3H), 5.80 (m, 1H), 5.60 (dt, 1H), 5.12 (dd, 1H), 4.70 (m, 1H), 4.50 (d, 1H), 4.42 (dd, 1H), 4.05 (m, 1H), 3.96 (dd, 1H), 2.56-2.94 (m, 3H), 2.28-2.46 (m, 2H), 1.66-1.80 (m, 2H), 1.50 (d, 3H), 1.48 (d, 3H), 1.30-1.44 (m, 5H), 1.28 (s, 9H), 0.94-1.22 (m, 7H). |
| 214 | 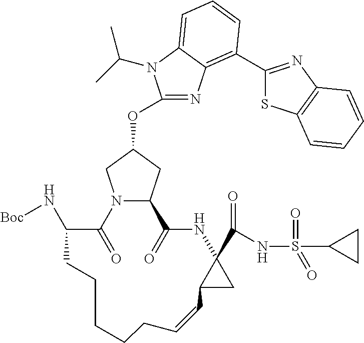 | Yield: 27%. ¹H-NMR (DMSO-d₆), δ: 11.14 (s, 1H), 8.94 (s, 1H), 8.20 (d, 1H), 8.15 (d, 1H), 7.68 (d, 1H), 7.54 (dd, 1H), 7.44 (dd, 1H), 7.29 (dd, 1H), 7.15 (d, 1H), 5.93 (m, 1H), 5.61 (dt, 1H), 5.13 (dd, 1H), 4.77 (m, 1H), 4.59 (d, 1H), 4.46 (dd, 1H), 4.00-4.18 (m, 1H), 2.78-2.96 (m, 2H), 2.58-2.66 (m, 2H), 2.28-2.56 (dd, 1H), 1.62-1.80 (m, 3H), 1.58 (d, 2H), 1.53 (d, 3H), 1.52 (d, 3H), 1.30-1.46 (m, 5H), 1.27 (s, 9H), 0.98-1.24 (m, 7H). |
| 215 | 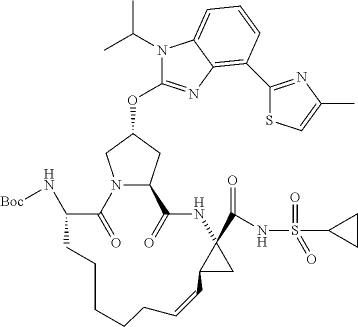 | Yield: 60%. ¹H-NMR (DMSO-d₆), δ: 11.12 (s, 1H), 8.93 (s, 1H) 7.97 (d, 1H), 7.55 (d, 1H), 7.36 (s, 1H), 7.20 (dd, 1H), 7.15 (d, 1H), 5.83 (m, 1H), 5.61 (dt, 1H), 5.12 (dd, 1H), 4.73 (m, 1H), 4.53 (d, 1H), 4.43 (dd, 1H), 3.96-4.08 (m, 2H), 2.54-2.96 (m, 3H), 2.45 (d, 3H), 2.36-2.48 (m, 1H), 2.32 (dd, 1H), 1.64-1.80 (m, 2H), 1.51 (d, 3H), 1.49 (d, 3H), 1.30-1.42 (m, 5H), 1.28 (s, 9H), 0.96-1.24 (m, 7H). |
| 216 | 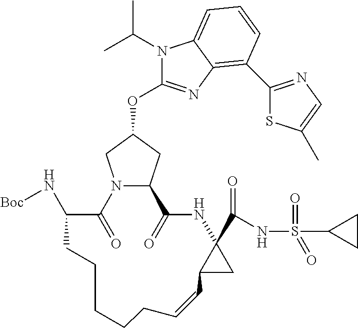 | Yield: 61%. ¹H-NMR (DMSO-d₆), δ: 11.12 (s, 1H), 8.92 (s, 1H), 7.93 (d, 1H), 7.61 (s, 1H), 7.54 (d, 1H), 7.19 (dd, 1H), 7.15 (d, 1H), 5.85 (m, 1H), 5.61 (dt, 1H), 5.12 (dd, 1H), 4.72 (m, 1H), 4.52 (d, 1H), 4.43 (dd, 1H), 3.98-4.08 (m, 2H), 2.58-2.94 (m, 3H), 2.52 (d, 3H), 2.40-2.48 (m, 1H), 2.32 (dd, 1H), 1.64-1.78 (m, 2H), 1.58 (d, 2H), 1.51 (d, 3H), 1.49 (d, 3H), 1.30-1.44 (m, 5H), 1.28 (s, 9H), 0.98- 1.14 (m, 7H) |
| 217 | 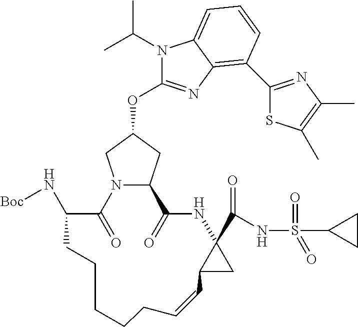 | Yield: 47%. ¹H-NMR (DMSO-d₆), δ: 11.12 (s, 1H), 8.92 (s, 1H), 7.91 (d, 1H), 7.51 (d, 1H), 7.17 (dd, 1H), 7.14 (d, 1H), 5.84 (m, 1H), 5.61 (dt, 1H), 5.12 (dd, 1H), 4.71 (m, 1H), 4.51 (d, 1H, 4.43 (dd, 1H), 3.98-4.06 (m, 2H), 2.86-2.94 (m, 1H), 2.72- 2.80 (m, 1H), 2.56-2.69 (m, 1H), 2.41 (s, 3H), 2.34 (s, 3H), 2.28-2.32 (dd, 1H), 1.64-1.80 (m, 2H), 1.58 (d, 2H), 1.50 (d, 3H), 1.48 (d, 3H), 1.31-1.44 (m, 5H), 1.28 (s, 9H), 0.96-1.22 (m, 7H). |
| 218 | 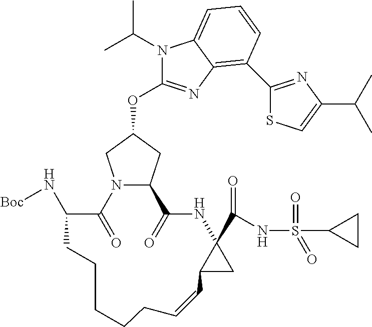 | Yield: 70%. ¹H-NMR (DMSO-d₆), δ: 11.12 (s, 1H), 8.93 (s, 1H), 7.97 (d, 1H), 7.55 (d, 1H), 7.35 (s, 1H), 7.20 (dd, 1H) 7.14 (d, 1H), 5.82 (m, 1H), 5.61 (dt, 1H), 5.12 (dd, 1H), 4.73 (m, 1H), 4.54 (d, 1H), 4.43 (dd, 1H), 3.96-4.08 (m, 2H), 3.12 (m, 1H), 2.86-2.94 (m, 1H), 2.78-2.84 (m, 1H), 2.58- 2.68 (m, 1H), 2.40-2.50 (m, 1H), 2.32 (dd, 1H), 1.64-1.78 (m, 2H), 1.58 (d, 2H), 1.34-1.44 (m, 5H), 1.32 (d, 6H), 1.27 (s, 9H), 0.98-1.24 (m, 7H). |
| 219 | 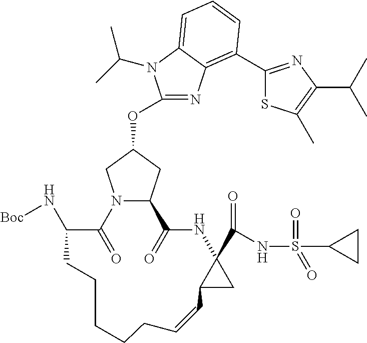 | Yield: 58%. ¹H-NMR (DMSO-d₆), δ: 11.12 (s, 1H), 8.92 (s, 1H), 7.93 (d, 1H), 7.51 (d, 1H), 7.18 (dd, 1H), 7.14 (d, 1H), 5.83 (m, 1H), 5.61 (dt, 1H), 5.12 (dd, 1H), 4.71 (m, 1H), 4.51 (d, 1H), 4.43 (dd, 1H), 3.98-4.08 (m, 2H), 3.16 (m, 1H), 2.88- 2.94 (m, 1H), 2.70-2.80 (m, 1H), 2.54-2.66 (m, 1H), 2.42 (s, 3H), 2.32 (dd, 1H), 1.64-1.80 (m, 2H), 1.58 (d, 2H), 1.51 (d, 3H), 1.48 (d, 3H), 1.31- 1.44 (m, 5H), 1.27 (s, 9H), 1.27 (d, 6H), 0.96-1.24 (m, 7H). |
| 220 | 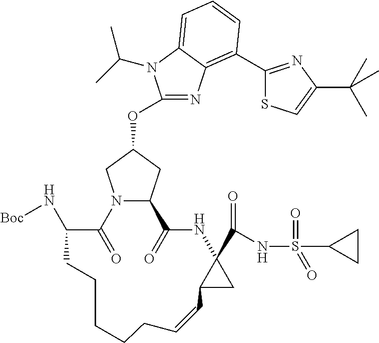 | Yield: 64%. ¹H-NMR (DMSO-d₆), δ: 11.12 (s, 1H), 8.93 (s, 1H), 7.98 (d, 1H), 7.55 (d, 1H), 7.36 (s, 1H), 7.21 (dd, 1H), 7.14 (d, 1H), 5.82 (m, 1H), 5.60 (dt, 1H), 5.11 (dd, 1H), 4.73 (m, 1H), 4.54 (d, 1H), 4.43 (dd, 1H), 3.96-4.04 (m, 2H), 2.86-2.94 (m, 1H), 2.78-2.84 (m, 1H), 2.58-2.66 (m, 1H), 2.38-2.45 (m, 1H), 2.32 (dd, 1H), 1.64-1.78 (m, 2H), 1.58 (d, 2H), 1.51 (d, 3H), 1.49 (d, 3H), 1.30- 1.44 (m, 12 H), 1.27 (s, 9H), 0.98-1.24 (m, 7H). |
| 221 | 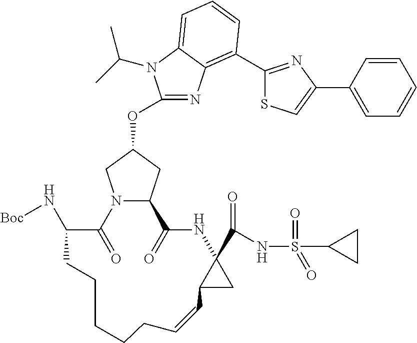 | Yield: 65%. ¹H-NMR (DMSO-d₆), δ: 11.13 (s, 1H), 8.94 (s, 1H), 8.23 (s, 1H), 8.14 (d, 1H), 8.07- 8.11 (m, 2H), 7.60 (d, 1H), 7.49 (dd, 2H), 7.37 (dd, 1H), 7.27 (dd, 1H), 7.15 (d, 1H), 5.87 (m, 1H), 5.61 (dt, 1H), 5.12 (dd, 1H), 4.75 (m, 1H), 4.59 (d, 1H), 4.44 (dd, 1H), 3.98-4.08 (m, 2H), 2.78-2.96 (m, 2H), 2.60-2.69 (m, 1H), 2.29-2.38 (m, 1H), 1.64-1.80 (m, 2H), 1.58 (d, 2H), 1.53 (d, 3H), 1.51 (d, 3H), 1.31-1.46 (m, 5H), 1.28 (s, 9H), 0.96-1.24 (m, 7H). |
| 222 | 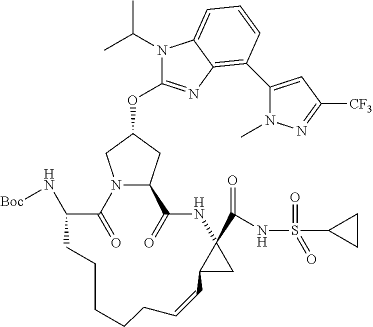 | Yield: 63%. ¹H-NMR (DMSO-d₆), δ: 11.07 (s, 1H), 8.96 (s, 1H), 7.61 (dd, 1H), 7.18-7.24 (m, 2H), 7.12 (d, 1H), 6.87 (s, 1H), 5.70 (m, 1H), 5.61 (dt, 1H), 5.09 (dd, 1H), 4.72 (m, 1H), 4.60 (d, 1H), 4.43 (dd, 1H), 3.96-4.02 (m, 1H), 3.92 (s, 3H), 3.88 (dd, 1H), 2.86-2.94 (m, 1H), 2.58-2.66 (m, 2H), 2.28-2.39 (m, 2H), 1.65-1.78 (m, 2H), 1.56- 1.62 (m, 2H), 1.55-1.62 (m, 2H), 1.48 (d, 6H), 1.26-1.42 (m, 5H), 1.18 (s, 9H), 0.92-1.18 (m, 7H). |
| 223 | 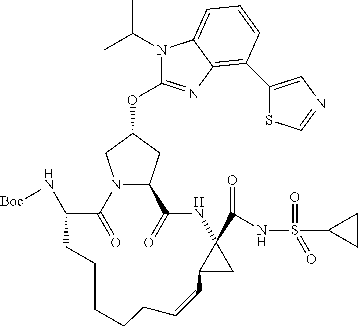 | Yield: 55%. ¹H-NMR (DMSO-d₆), δ: 11.11 (s, 1H), 9.10 (s, 1H), 8.93 (s, 1H), 7.52 (d, 1H), 7.47 (d, 1H), 7.16 (dd, 1H), 7.14 (d, 1H), 5.82 (m, 1H), 5.61 (dt, 1H), 5.12 (dd, 1H), 4.71 (m, 1H), 4.53 (d, 1H), 4.42 (dd, 1H), 4.00-4.06 (m, 1H), 3.96 (dd, 1H), 2.84-2.94 (m, 1H), 2.72-2.84 (m, 1H), 2.56- 2.72 (m, 1H), 2.38-2.48 (m, 1H), 2.32-2.38 (m, 1H), 1.64-1.82 (m, 2H), 1.58 (d, 2H), 1.50 (d, 3H), 1.48 (d, 3H), 1.30-1.44 (m, 5H), 1.27 (s, 9H), 0.96- 1.24 (m, 7H). |
| 224 | 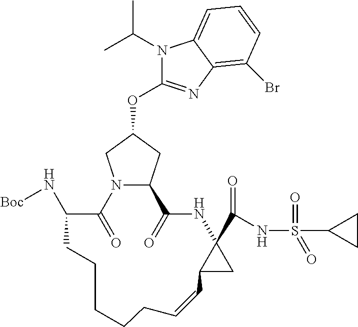 | Yield: 39%. ¹H-NMR (DMSO-d₆), δ: 11.07 (s, 1H), 8.93 (s, 1H), 7.49 (d, 1H), 7.31 (d, 1H), 7.14 (d, 1H), 7.03 (dd, 1H), 5.75 (m, 1H), 5.60 (dt, 1H), 5.12 (dd, 1H), 4.66 (m, 1H), 4.50 (d, 1H), 4.39 (dd, 1H), 4.00-4.06 (m, 1H), 3.91 (dd, 1H), 2.86- 2.94 (m, 1H), 2.56-2.76 (m, 2H), 2.30-2.44 (m, 2H), 1.64-1.78 (m, 2H), 1.58 (d, 2H), 1.47 (d, 3H), 1.45 (d, 3H), 1.30-1.42 (m, 5H), 1.26 (s, 9H), 0.96- 1.24 (m, 7H). |
| 225 | 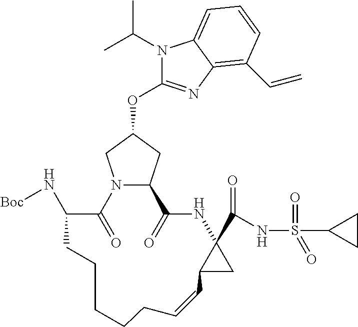 | Yield: 19%. ¹H-NMR (DMSO-d₆), δ: 11.07 (s, 1H), 8.93 (s, 1H), 7.36 (d, 1H), 7.22 (d, 1H), 7.04- 7.14 (m, 3H), 6.31 (dd, 1H), 5.76 (m, 1H), 5.60 (dt, 1H), 5.39 (dd, 1H), 5.12 (dd, 1H), 4.17 (m, 1H), 4.48 (d, 1H), 4.39 (dd, 1H), 4.0-4.07 (m, 1H), 3.93 (dd, 1H), 2.85-2.95 (m, 1H), 2.55-2.78 (m, 2H), 2.29-2.45 (m, 2H), 1.65-1.75 (m, 2H), 1.57 (d, 2H), 1.47 (d, 3H), 1.1.45 (d, 3H), 1.32-1.42 (m, 5H), 1.28 (s, 9H), 0.94-1.24 (m, 7H). |
| 226 | 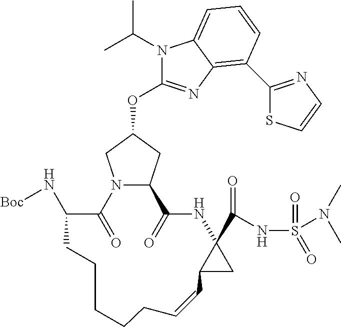 | Yield: 82%. ¹H-NMR (DMSO-d₆), δ: 10.83 (s, 1H), 8.98 (s, 1H), 8.01 (d, 1H), 7.94 (d, 1H), 7.83 (d, 1H), 7.57 (d, 1H), 7.22 (dd, 1H), 7.15 (d, 1H), 5.85 (m, 1H), 5.63 (dt, 1H), 5.08 (dd, 1H), 4.74 (m, 1H), 4.56 (d, 1H), 4.45 (dd, 1H), 3.95-4.08 (m, 2H), 2.76-2.84 (m, 1H), 2.74 (s, 6H), 2.60-2.63 (m, 1H), 2.40-2.48 (m, 1H), 2.28 (dd, 1H), 1.62 (m, 2H), 1.54-1.58 (m, 1H), 1.51 (d, 3H), 1.50 (d, 3H), 1.31-1.44 (m, 5H), 1.27 (s, 9H), 1.04-1.24 (m, 3H). Yield: 89%%. ¹H-NMR (DMSO-d₆), δ: 10.79 (s, 1H), 8.96 (s, 1H), 8.28 (s, 1H), 7.73 (d, 1H), 7.62 (d, 1H), 7.41 (s, 1H), 7.21 (dd, 1H), 7.14 (d, 1H), 5.81 (m, 1H), 5.61 (dt, 1H), 5.08 (dd, 1H), 4.73 (m, 1H), 4.50 (d, 1H), 4.41 (dd, 1H), 4.02-4.06 (m, 1H), 3.93 (dd, 1H), 2.73-2.78 (m, 1H), 2.73 (s, 6H), 2.28-2.44 (m, 2H), 1.62-1.78 (m, 2H), 1.55 (m, 2H), 1.49 (d, 3H), 1.48 (d, 3H), 1.30-1.42 (m, 5H), 1.27 (s, 9H), 1.00-1.24 (m, 3H). |
| 227 | 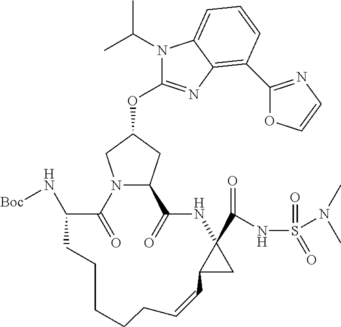 | Yield: 89%%. ¹H-NMR (DMSO-d₆), δ: 10.79 (s, 1H), 8.96 (s, 1H), 8.28 (s, 1H), 7.73 (d, 1H), 7.62 (d, 1H), 7.41 (s, 1H), 7.21 (dd, 1H), 7.14 (d, 1H), 5.81 (m, 1H), 5.61 (dt, 1H), 5.08 (dd, 1H), 4.73 (m, 1H), 4.50 (d, 1H), 4.41 (dd, 1H), 4.02-4.06 (m, 1H), 3.93 (dd, 1H), 2.73-2.78 (m, 1H), 2.73 (s, 6H), 2.28-2.44 (m, 2H), 1.62-1.78 (m, 2H), 1.55 (m, 2H), 1.49 (d, 3H), 1.48 (d, 3H), 1.30-1.42 (m, 5H), 1.27 (s, 9H), 1.00-1.24 (m, 3H). |
| 228 | 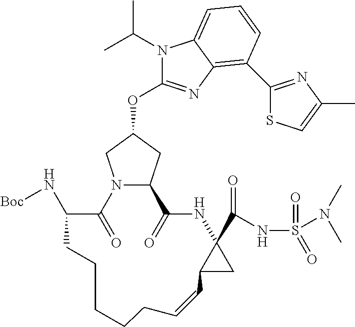 | Yield: 69.6%. ¹H-NMR (DMSO-d₆), δ: 10.82 (s, 1H), 8.97 (s, 1H), 7.96 (d, 1H), 7.55 (d, 1H), 7.36 (s, 1H), 7.20 (dd, 1H), 7.15 (d, 1H), 5.83 (m, 1H), 5.62 (dt, 1H), 5.08 (dd, 1H), 4.73 (m, 1H), 4.54 (d, 1H), 4.45 (dd, 1H), 3.76-4.04 (m, 2H), 2.76-2.84 (dd, 1H), 2.74 (s, 6H), 2.45 (s, 3H), 2.38-2.48 (m, 1H), 2.28 (dd, 1H), 1.64-1.78 (m, 2H), 1.54-1.60 (m, 2H), 1.51 (d, 3H), 1.49 (d, 3H), 1.30-1.45 (m, 5H), 1.27 (s, 9H), 1.04-1.24 (m, 3H). |
| 229 | 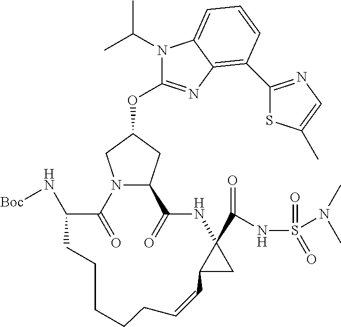 | Yield: 75%. ¹H-NMR (DMSO-d₆), δ: 10.82 (s, 1H), 8.97 (s, 1H), 7.93 (d, 1H), 7.61 (d, 1H), 7.53 (d, 1H), 7.19 (dd, 1H), 7.15 (d, 1H), 5.85 (m, 1H), 5.63 (dt, 1H), 5.08 (dd, 1H), 4.72 (m, 1H), 4.52 (d, 1H), 4.45 (dd, 1H), 3.98-4.06 (m, 2H), 2.80 (m, 1H), 2.74 (s, 6H), 2.60-2.68 (m, 1H), 2.52 (d, 3H), 2.42-2.48 (m, 1H), 2.29 (dd, 1H), 1.66-1.78 (m, 2H), 1.54-1.60 (m, 1H), 1.51 (d, 3H), 1.49 (d, 3H), 1.30-1.42 (m, 5H), 1.27 (s, 9H), 1.02-1.24 (m, 3H). |
| 230 | 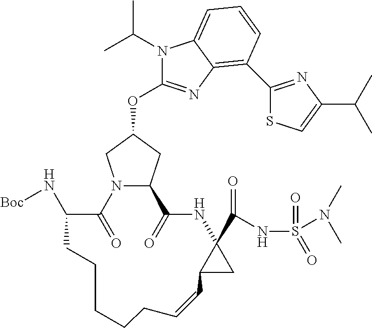 | Yield: 71%. ¹H-NMR (DMSO-d₆), δ: 10.82 (s, 1H), 8.98 (s, 1H), 7.97 (d, 1H), 7.55 (d, 1H), 7.35 (s, 1H), 7.20 (dd, 1H), 7.15 (d, 1H), 5.83 (m, 1H), 5.62 (dt, 1H), 5.08 (dd, 1H), 4.73 (m, 1H), 4.55 (d, !H), 4.48 (dd, 1H), 4.00-4.05 (m, 1H), 3.98 (dd, 1H), 3.12 (m, 1H), 2.80 (dd, 1H), 2.74 (s, 6H), 2.58-2.70 (m, 1H), 2.38-2.70m, 1H), 2.29 (dd, 1H), 1.62-1.78 (m, 2H), 1.53-1.58 (m, 2H), 1.51 (d, 3H), 1.49 (d, 3H), 1.34-1.42 (m, 5H), 1.32 (d, 6H), 1.27 (s, 9H), 1.04-1.24 (m, 3H). |
| 231 | 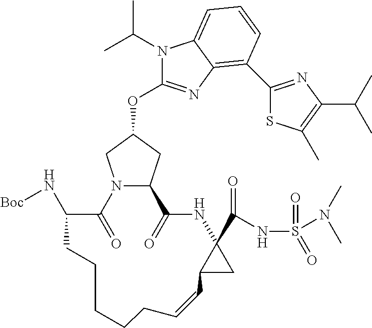 | Yield: 68%. ¹H-NMR (DMSO-d₆), δ: 10.81 (s, 1H), 8.97 (s, 1H), 7.93 (d, 1H), 7.51 (d, 1H), 7.18 (dd, 1H), 7.14 (d, 1H), 5.83 (m, 1H), 5.62 (dt, 1H), 5.08 (dd, 1H), 4.71 (m, 1H), 4.50 (d, 1H), 4.45 (dd, 1H), 3.98-4.06 (m, 2H), 3.14 (m, 1H), 2.74 (s, 6H), 2.78 (m, 1H), 2.58-2.69 (m, 1H), 2.42 (s, 3H), 2.43-2.46 (m, 1H), 2.28 (dd, 1H), 1.64-1.80 (m, 2H), 1.53-1.60 (m, 2H), 1.51 (d, 3H), 1.49 (m, 3H), 1.30-1.44 (m, 5H), 1.26-1.28 (d, s, 15H), 1.10-1.24 (m, 3H). |
| 232 | 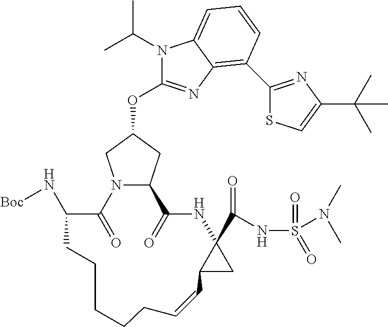 | Yield: 63%. ¹H-NMR (DMSO-d₆), δ: 10.82 (s, 1H), 8.98 (s, 1H), 7.98 (d, 1H), 7.55 (d, 1H), 7.36 (s, 1H), 7.21 (dd, 1H), 7.14 (d, 1H), 5.83 (m, 1H), 5.63 (dt, 1H), 5.08 (dd, 1H), 4.73 (m, 1H), 4.55 (d, 1H), 4.45 (dd, 1H), 4.02-4.06 (m, 1H), 3.98 (dd, 1H), 2.80 (dd, 1H), 2.74 (s, 6H), 2.56-2.68 (m, 1H), 2.38-2.46 (m, 1H), 2.29 (dd, 1H), 1.64-1.80 (m, 2H), 1.54-1.58 (m, 1H), 1.51 (d, 3H), 1.49 (d, 3H), 1.30-1.44 (m and s, 14H), 1.27 (s, 9H), 1.04- 1.24 (m, 3H). |
| 233 | 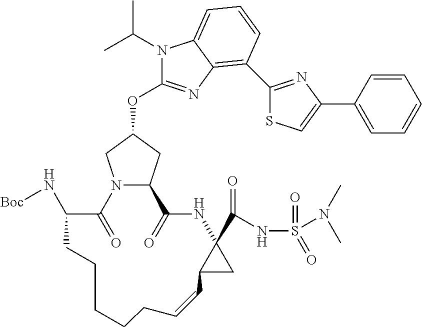 | Yield: 63%. ¹H-NMR (DMSO-d₆), δ: 10.83 (s, 1H), 8.98 (s, 1H), 8.24 (s, 1H), 8.14 (d, 1H), 8.10 (m, 2H), 7.61 (d, 1H), 7.49 (m, 2H), 7.37 (m, 1H), 7.27 (dd, 1H), 7.16 (d, 1H), 5.87 (m, 1H), 5.63 (dt, 1H), 5.08 (dd, 1H), 4.75 (m, 1H), 4.59 (d, 1H), 4.47 (dd, 1H), 4.02-4.08 (m, 1H), 3.99 (dd, 1H), 2.82 (dd, 1H), 2.74 (s, 6H), 2.60-2.68 (m, 1H), 2.42-2.48 (m, 1H), 2.30 (dd, 1H), 1.62-1.78 (m, 2H), 1.54-1.60 (m, 1H), 1.53 (d, 3H), 1.51 (d, 3H), 1.30-1.48 (m, 5H), 1.27 (s, 9H), 1.02-1.24 (m, 3H). |
| 234 | 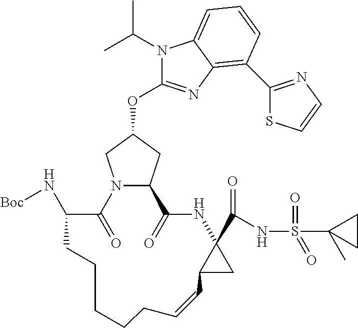 | Yield: 61%. ¹H-NMR (DMSO-d₆), δ: 10.88 (s, 1H), 9.07 (s, 1H), 8.01 (d, 1H), 7.95 (d, 1H), 7.83 (d, 1H), 7.57 (d, 1H), 7.22 (dd, 1H), 7.15 (d, 1H), 5.86 (m, 1H), 5.61 (dt, 1H), 5.06 (dd, 1H), 4.74 (m, 1H), 4.57 (d, 1H), 4.47 (dd, 1H), 3.96-4.08 (m, 2H), 2.83 (dd, 1H), 2.54-2.68 (m, 1H), 2.40-2.48 (m, 1H), 2.33 (dd, 1H), 1.66-1.80 (m, 2H), 1.55- 1.60 (m, 2H), 1.52 (d, 3H), 1.50 (d, 3H), 1.02-1.44 (m, 21H), 0.82-0.90 (m, 2H). |
| 235 | 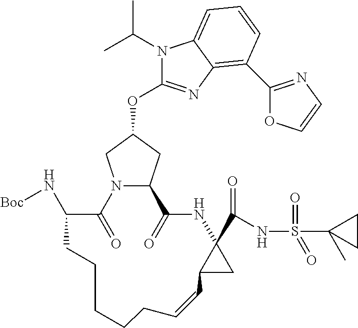 | Yield: 52%. ¹H-NMR (DMSO-d₆), δ: 10.85 (s, 1H), 9.05 (s, 1H), 8.28 (d, 1H), 7.74 (d, 1H), 7.62 (d, 1H), 7.41 (d, 1H), 7.21 (dd, 1H), 7.14 (d, 1H), 5.82 (m, 1H), 5.60 (dt, 1H), 5.06 (dd, 1H), 4.73 (m, 1H), 4.50 (d, 1H), 4.44 (dd, 1H), 4.00-4.06 (m, 1H), 3.93 (dd, 1H), 2.75 (dd, 1H), 2.56-2.68 (m, 1H), 2.31-2.42 (m, 2H), 1.66-1.78 (m, 2H), 1.55- 1.68 (m, 2H), 1.48 (d, 6H), 1.30-1.42 (m, 9H), 1.26 (s, 9H), 1.02-1.22 (m, 3H), 0.80-0.90 (m, 2H). |
| 236 | 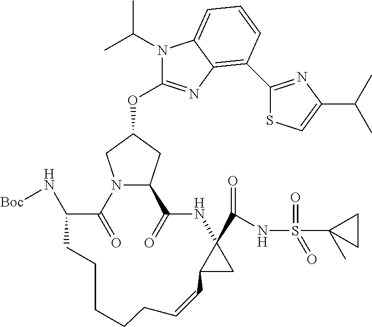 | Yield: 58%. ¹H-NMR (DMSO-d₆), δ: 10.88 (s, 1H), 9.07 (s, 1H), 7.97 (d, 1H), 7.55 (d, 1H), 7.35 (s, 1H), 7.20 (dd, 1H), 7.15 (d, 1H), 5.84 (m, 1H), 5.61 (dt, 1H), 5.06 (dd, 1H), 4.73 (m, 1H), 4.55 (d, 1H), 4.47 (dd, 1H), 3.96-4.06 (m, 2H), 3.12 (m, 1H), 2.80 (dd, 1H), 2.56-2.68 (m, 1H), 2.38-2.48 (m, 1H), 2.32 (dd, 1H), 1.68-1.80 (m, 2H), 1.53- 1.60 (m, 2H), 1.51 (d, 3H), 1.49 (d, 3H), 1.32-1.44 (m, 8H), 1.32 (d, 6H), 1.27 (s, 9H), 1.02-1.24 (m, 3H), 0.82-0.90 (m, 2H). |
| 237 | 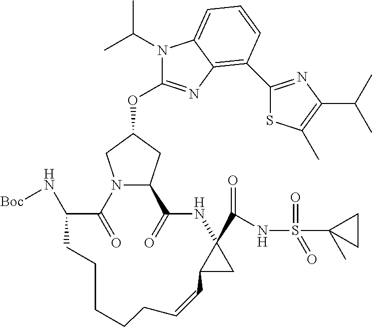 | Yield: 57%. ¹H-NMR (DMSO-d₆), δ: 10.87 (s, 1H), 9.06 (s, 1H), 7.93 (d, 1H), 7.51 (d, 1H), 7.18 (dd, 1H), 7.14 (d, 1H), 5.84 (m, 1H), 5.61 (dt, 1H), 5.06 (dd, 1H), 4.71 (m, 1H), 4.42-4.54 (m, 2H), 3.98-4.08 (m, 2H), 3.14 (m, 1H), 2.78 (dd, 1H), 2.54-2.68 (m, 1H), 2.43-2.48 (m, 1H), 2.42 (s, 3H), 2.31 (dd, 1H), 1.66-1.80 (m, 2H), 1.53-1.60 (m, 2H), 1.50 (d, 3H), 1.49 (d, 3H), 1.31-1.45 (m, 8H), 1.06-1.32 (m, 18H), 0.81-0.90 (m, 2H). |

### 2.22 Synthesis of Compounds 238-253

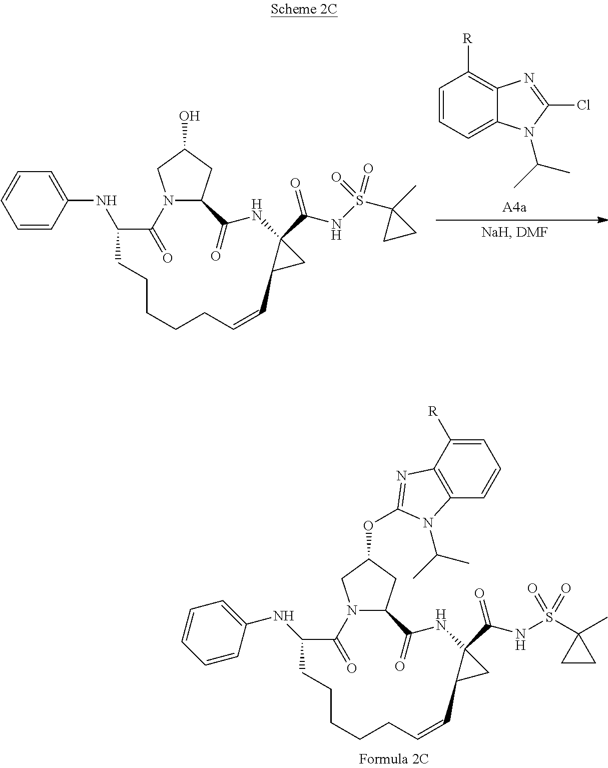

### Synthesis of Intermediate A4

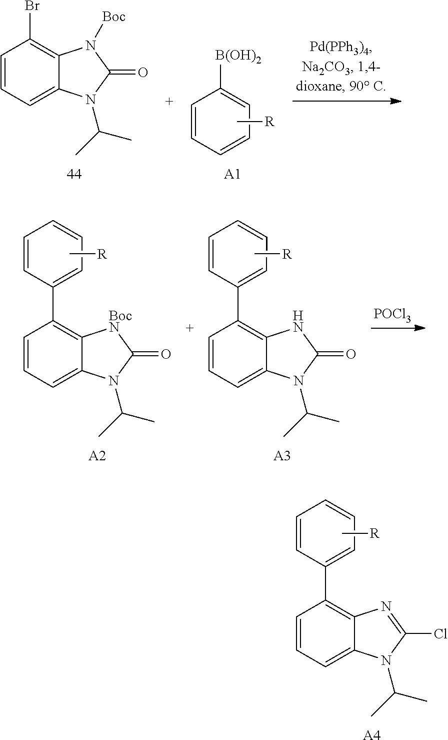

A flask was charged with compound 44 (1.0 eq.), compound A1 (2 eq.), Pd(PPh₃)₄ (0.1 eq.), Na₂CO₃ (2 eq.), 1,4-dioxane (2 mL) and one drop of water. After the flask was purged with nitrogen, the mixture was stirred overnight at 90° C. The mixture was filtered and concentrated, and then purified with prep-TLC to give compound A2 & and A3 respectively. A flask charged with a mixture of compounds A2, A3 and POCl₃. The mixture was stirred at 100° C. for 8 hrs, then was poured into ice-water, extracted with EtOAc, washed by saturated aq. NaHCO₃ and brine, dried over anhydrous Na₂SO₄, concentrated to afford compound A4.

Compounds 238-253 were made according to Scheme 2C. To a suspension of NaH (60% dispersion in mineral oil, 8 eq.) in DMF (2 mL) was added compound 19 (50 mg, 0.089 mmol) at 0° C. After stirring for 2 hrs at 0-5° C., compound A4a (1.2 eq) was added, the resulting mixture was warmed to room temperature and stirred for 12 hrs. After completion of the reaction, the mixture was cooled by ice water, acidified with aq HCl (1 N) to pH=5-6, then the mixture was extracted with ethyl acetate (20 mL×3), the organic layers were combined, washed with brine, dried over anhydrous sodium sulfate, the solvent was removed under reduced pressure, the residue was purified by preparative HPLC to give Formula 2C.

TABLE 4
Compounds prepared according to Scheme 2C.

| Compound | Structure | Yield |
|---|---|---|
| 238 | 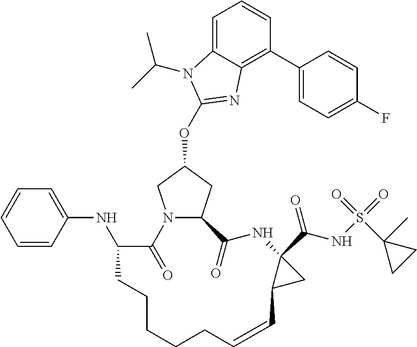 | 9.8 mg, 14%. MS (ESI) m/z (M + H)⁺ 811.3. |
| 239 | 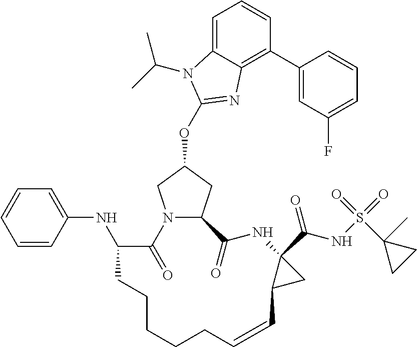 | 9.7 mg, 14%. MS (ESI) m/z (M + H)⁺ 811.3. |
| 240 | 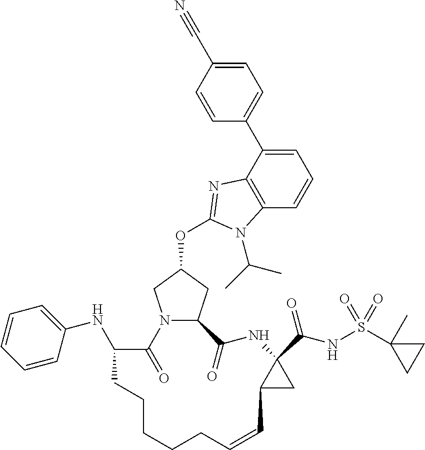 | 19.4 mg, 27%. MS (ESI) m/z (M + H)⁺ 818.3. |
| 241 | 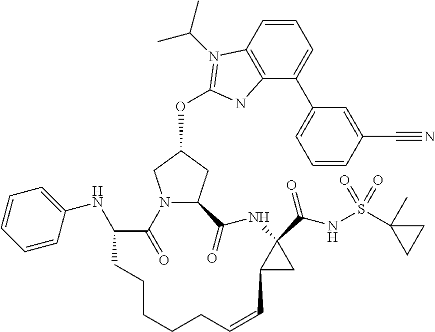 | 9.2 mg, 13%. MS (ESI) m/z (M + H)⁺ 818.3. |
| 242 | 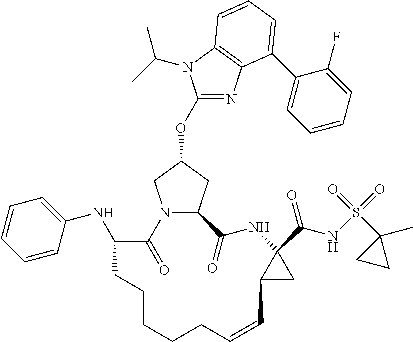 | 6.2 mg, 9%. MS (ESI) m/z (M + H)⁺ 810.9. |
| 243 | 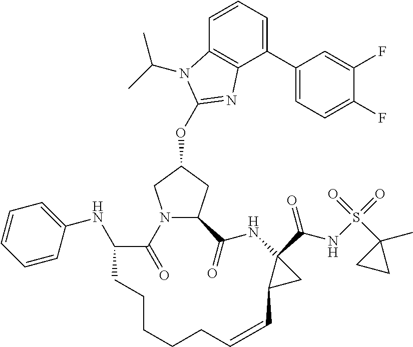 | 9.8 mg, 13%. MS (ESI) m/z (M + Na)⁺ 851 |
| 244 | 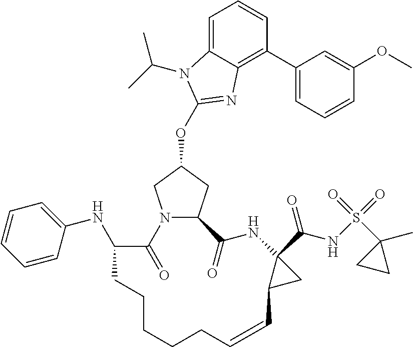 | 9.9 mg, 14%. MS (ESI) m/z (M + H)⁺ 823 |
| 245 | 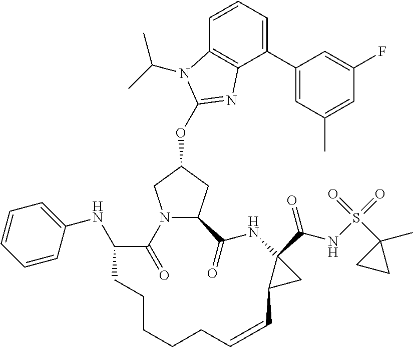 | 9.4 mg, 16%. MS (ESI) m/z (M + H)⁺ 825 |
| 246 | 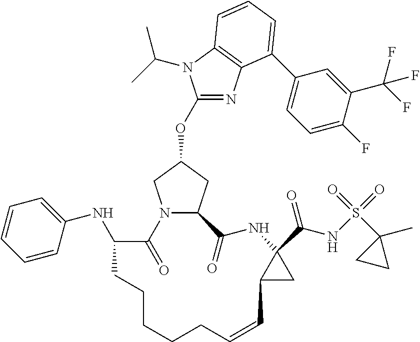 | 6.7 mg, 9%. MS (ESI) m/z (M + H)⁺ 879.3. |
| 247 | 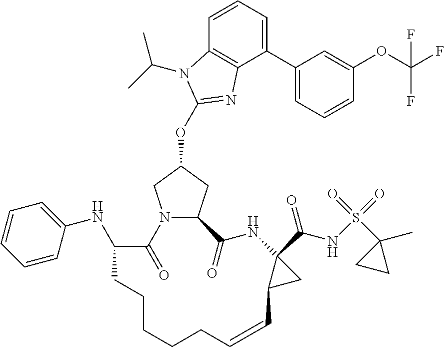 | 5.3 mg, 7%. MS (ESI) m/z (M + Na)⁺ 899.3. |
| 248 | 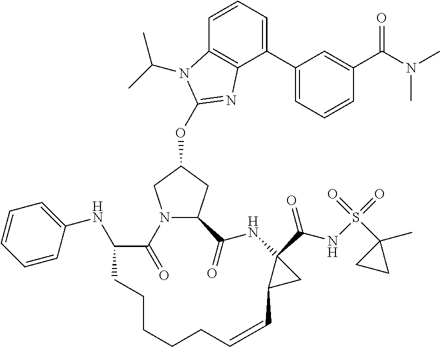 | 5.3 mg, 7%. MS (ESI) m/z (M + H)⁺ 864.3 |
| 249 | 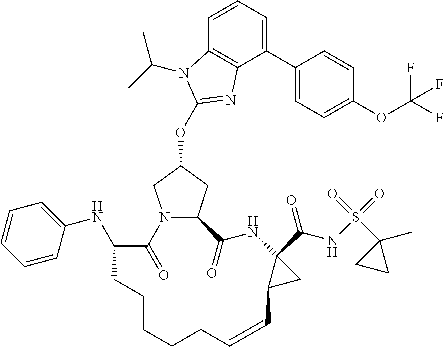 | 23 mg, 30%. MS (ESI) m/z (M + H)⁺ 877.3 |
| 250 | 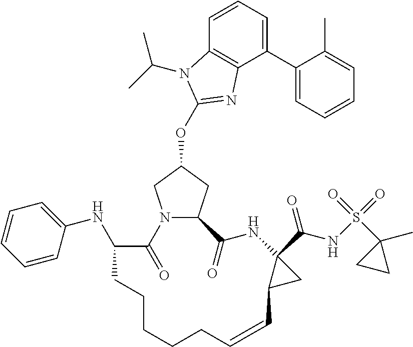 | 5 mg, 7%. MS (ESI) m/z (M + H)⁺ 807.2 |
| 251 | 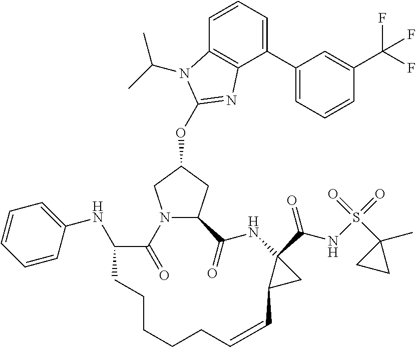 | 20.3 mg, 27%. MS (ESI) m/z (M + H)⁺ 861.3 |
| 252 | 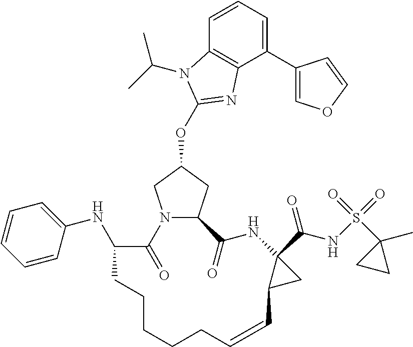 | 8.4 mg, 12%. MS (ESI) m/z (M + H)⁺ 783.3 |
| 253 | 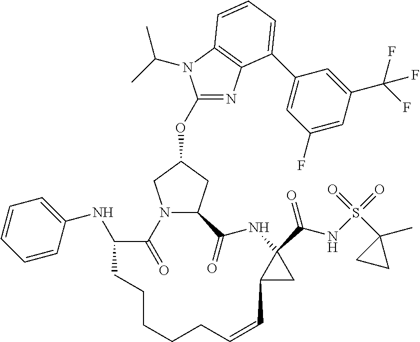 | 8.8 mg, 12%. MS (ESI) m/z (M + H)⁺ 842.3 |

### 2.23 Synthesis of Compounds 254-261

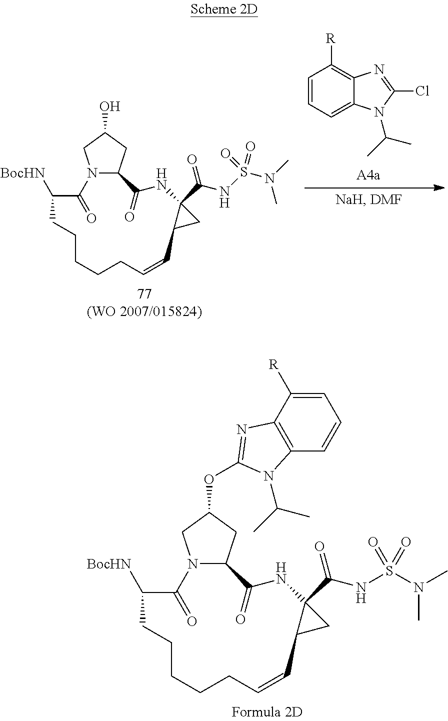

To a suspension of NaH (60% dispersion in mineral oil, 20 mg, 0.558 mmol) in DMF (1.5 mL) was added compound 77 (40 mg, 0.0697 mmol) at 0° C. After stirring for 1 hrs at 0-5° C., compound A4a (1.2 eq) was added, the resulting mixture was warmed to room temperature and stirred for 12 hrs. After completion of the reaction, the mixture was cooled by ice water, acidified with aq HCl (1 N) to pH=5-6, then the mixture was extracted with ethyl acetate (20 mL×3), the organic layers were combined, washed with brine, dried over anhydrous sodium sulfate, the solvent was removed under reduced pressure, the residue was purified by preparative HPLC to give Formula 2D.

TABLE 5
Compounds prepared according to Scheme 2D.

| Compound | Structure | Yield |
|---|---|---|
| 254 | 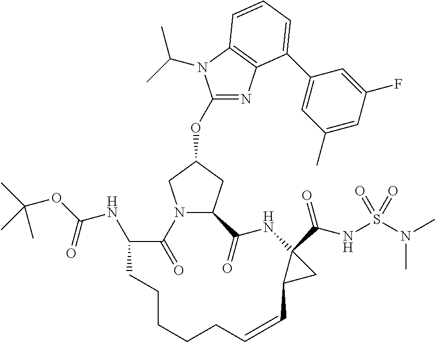 | 9.8 mg, 14%. MS (ESI) m/z (M + H)⁺ 811.3. |
| 255 | 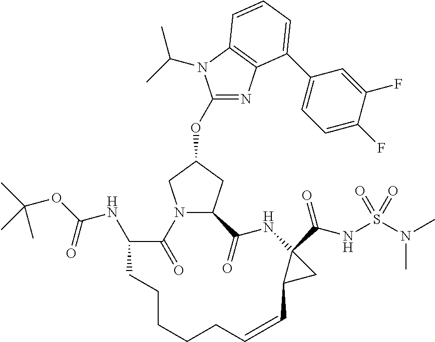 | 9.7 mg, 14%. MS (ESI) m/z (M + H)⁺ 811.3. |
| 256 | 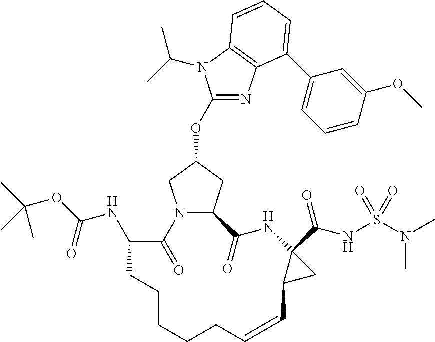 | 19.4 mg, 27%. MS (ESI) m/z (M + H)⁺ 818.3. |
| 257 | 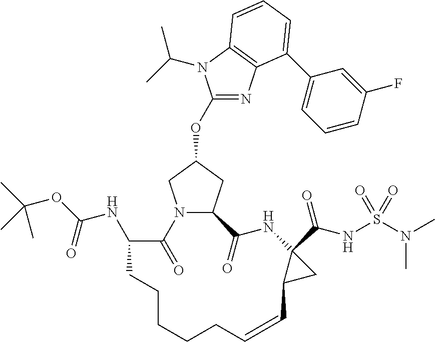 | 9.2 mg, 13%. MS (ESI) m/z (M + H)⁺ 818.3. |
| 258 | 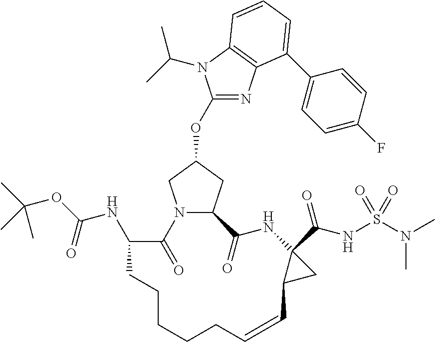 | 6.2 mg, 9%. MS (ESI) m/z (M + H)⁺ 810.9. |
| 259 | 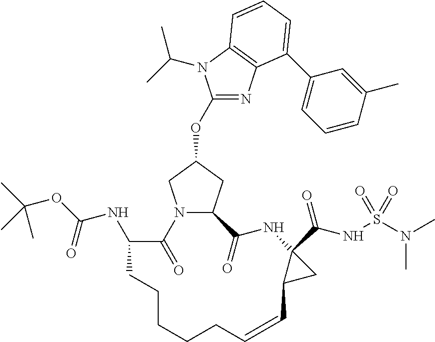 | 9.8 mg, 13%. MS (ESI) m/z (M + Na)⁺ 851 |
| 260 | 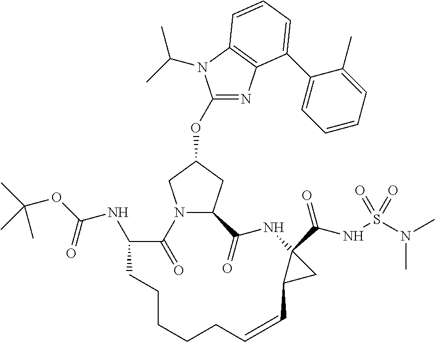 | 9.9 mg, 14%. MS (ESI) m/z (M + H)⁺ 823 |
| 261 | 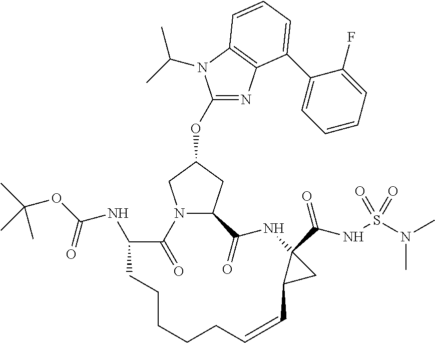 | 9.4 mg, 16%. MS (ESI) m/z (M + H)⁺ 825 |

### 2.24 Synthesis of Compound 288 (2.2)

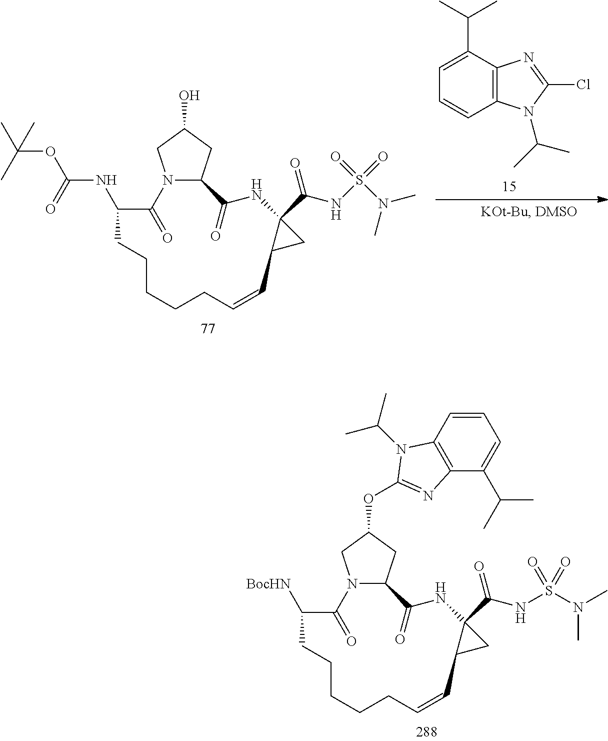

To a solution of compound 77 (100 mg, 0.175 mmol, 1 eq.) in 2 mL of DMSO at 0° C. was added KOt-Bu (118 mg, 1.05 mmol, 6 eq.) in portions, then the mixture was stirred for 30 min at ambient temperature. After that, compound 15 (50 mg, 0.21 mmol, 1.2 eq.) was added, the resulting mixture was stirred at ambient temperature for 12 hrs. The reaction was monitored by LCMS. After completion of the reaction, the mixture was cooled by ice water, acidified by aq. HCl (1 M) to pH=8, then the mixture was extracted by ethyl acetate (50 mL×3), the organic layers were combined, washed by brine, dried over anhydrous sodium sulfate, solvent was removed under reduced pressure and the residue was purified by prep-HPLC to give compound 288 (9.5 mg, 7.0%). MS (ESI) m/z (M+H)⁺ 772.3.

### 2.25 Synthesis of Compound 291 (2.3)

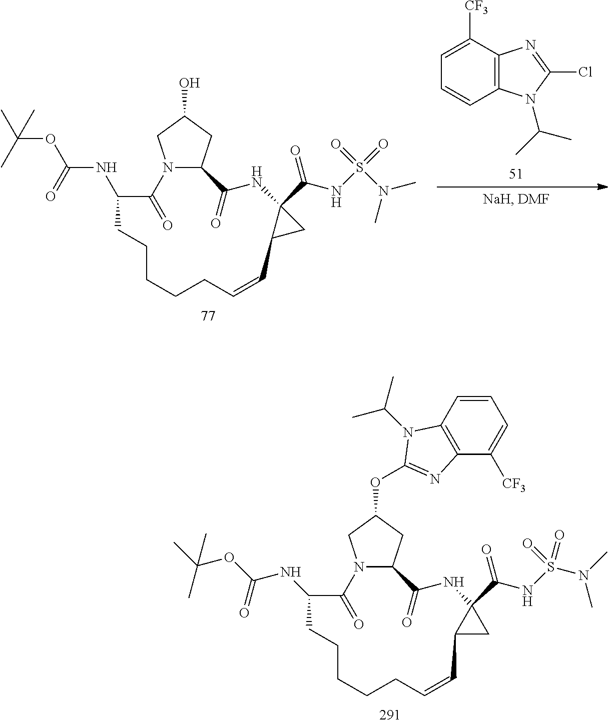

Compound 291 was prepared using the general procedure described above. Yield 12.4 mg (20%). MS (ESI) m/z (M+H)⁺ 798.4.

### 2.26 Synthesis of Compound 289 (2.4)

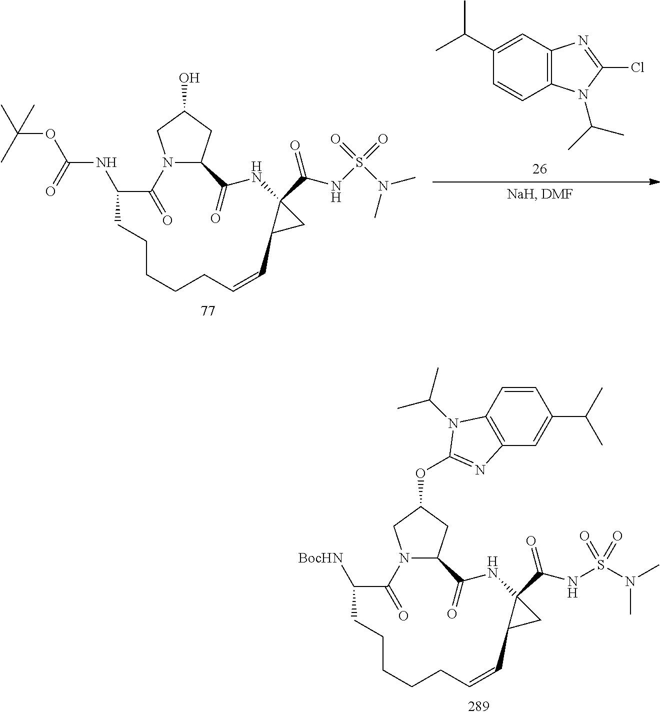

Compound 289 was prepared using the general procedure described above. Yield 10 mg (8%). MS (ESI) m/z (M+H)⁺ 772.4.

### 2.27 Synthesis of Compound 1223 and 1224 (2.5)

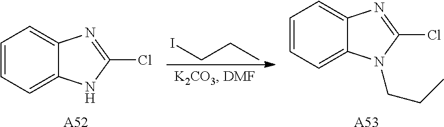

Preparation of compound 1223: To a solution of compound A52 (1.0 g, 6.5 mmol) in DMF (5 mL) were added K₂CO₃ (1.8 g, 13.1 mmol) and 1-iodopropane (2.2 g, 13.1 mmol), the reaction was stirred at room temperature overnight. The reaction mixture was monitored with TLC. After completion of the reaction, the reaction mixture was diluted with water, neutralized with aq. HCl (1 M), extracted with EtOAc (70 mL×3), the organic layers were combined, washed with brine, dried over anhydrous sodium sulfate, then the solvent was removed under reduced pressure, the residue was purified by silica gel column chromatography, eluting by petroleum ether and ethyl acetate (4:1), to afford compound A53 (1.19 g, 93%). ¹H NMR (400 MHz, CDCl₃): δ 7.61 (dd, 2.4 Hz, 1H), 7.23-7.17 (m, 3H), 4.07 (t, J=12Hz, 2H), 1.82-1.77 (m, 2H), 0.89 (t, J=12Hz, 3H).

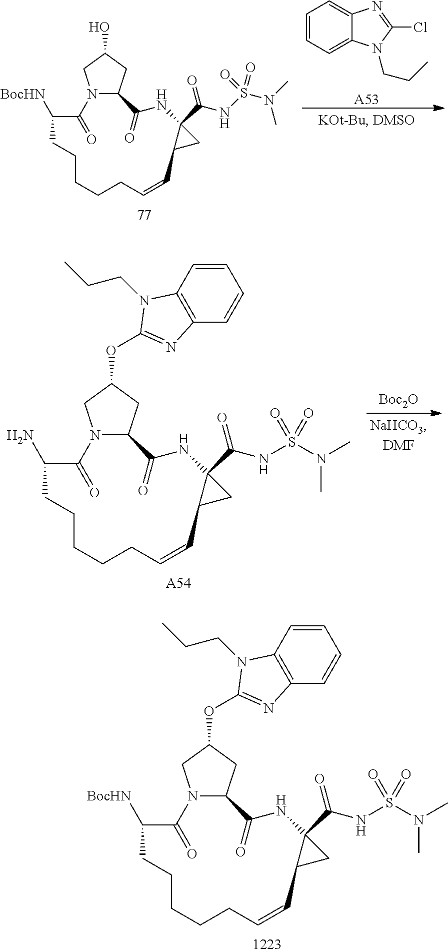

To a solution of compound 77 (1.0 eq.) in DMSO at 0° C. was added KOt-Bu (4.0 eq.), the mixture was stirred at 0° C. for 10 min, then compound A53 (1.1 eq.) was added and the reacting mixture was stirred at r.t. overnight. The reaction mixture was monitored with LCMS. After the material was consumed, the reaction mixture was diluted with water, neutralized with aq. HCl (1 M), extracted with EtOAc and washed with brine, the organic layer was concentrated and used directly without further purification.

The crude compound A54 (1.0 eq.) was dissolved in DMF. To the resulting solution was added Boc₂O (1.1 eq.) and NaHCO₃ (2.0 eq.) was added, the reaction mixture was stirred at room temperature overnight. The reaction mixture was monitored with TLC. After completion of the reaction, the mixture was diluted with water, neutralized with aq. HCl (1 M), extracted with EtOAc and washed with brine; the organic layer was concentrated in vacuo and purified by prep-HPLC to afford compound 1223 (53.4 mg, 41%). MS (ESI) m/z (M+H)⁺ 730.6.

Preparation of compound 1224: To a solution of compound A52 (1.0 g, 6.5 mmol) in DMF (5 mL) were added K₂CO₃ (1.8 g, 13.1 mmol) and 2-iodobutane (2.4 g, 13.1 mmol), the reaction was stirred at room temperature overnight. The reaction mixture was monitored with TLC. After completion of the reaction, the reaction mixture was diluted with water, neutralized with aq. HCl (1 M), extracted with EtOAc (70 mL×3), the organic layers were combined, washed with brine, dried over anhydrous sodium sulfate, then the solvent was removed under reduced pressure, the residue was purified by silica gel column chromatography, eluting by petroleum ether and ethyl acetate (4:1), to afford compound A55 (1 g, 73%).

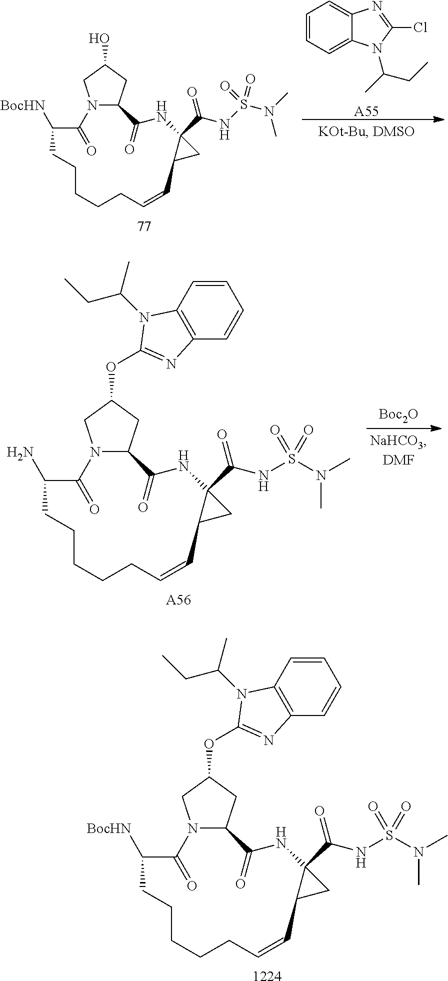

The same procedure for making compound 1223 as described in section 2.26 was used to prepare compound 1224 (48.5 mg, 37%). MS (ESI) m/z (M+H)⁺ 744.4.

### 2.28 Synthesis of Compound 290 (2.7)

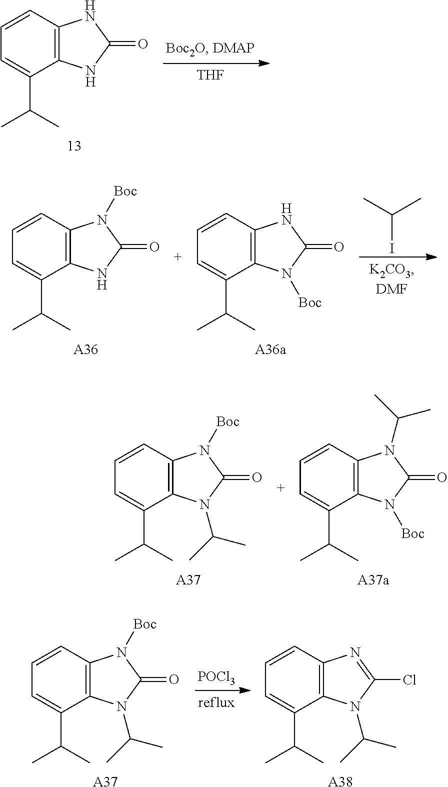

To a solution of compound 13 (1 g, 5.7 mmol) in anhydrous THF (20 mL) was added DMAP (700 mg, 3.3 mmol) and Boc₂O (1.3 g, 6 mmol). The reaction mixture was stirred at room temperature for 16 hrs. The mixture was diluted with water, extracted with EtOAc (70 mL×3). The combined organic layers was washed with brine, dried over Na₂SO₄, and evaporated to dryness under reduced pressure. The crude product was purified by silica gel column chromatography, eluting by petroleum ether and ethyl acetate (2:1), to give a mixture of compound A36 and A36a (1.4 g, 89%) as a white solid.

To a solution of compound A36 and A36a (1.4 g, 5 mmol) in DMF (9 mL) was added isopropyl iodide (1.7 g, 10 mmol) and K₂CO₃ (1.4 g, 10 mmol). The solution was stirred at room temperature for 16 hrs. The reaction mixture was diluted with water, extracted with EtOAc (70 mL×3). The combined organic layers was washed with brine, dried over Na₂SO₄, and evaporated to dryness under reduced pressure. Compound A37 was isolated by prep-TLC as a white solid and identified with NOE analysis (160 mg, 10%).

A flask (50 mL) was charged with compound A37 (160 mg, 0.5 mmol) and POCl₃ (4 mL). To the mixture was added TEA (50 mg, 0.5 mmol). The resulting mixture was stirred at 110° C. for 8 hrs. After the material was consumed, the mixture was diluted with EtOAc (100 mL), neutralized with saturated aq. NaHCO₃, washed with water and brine, dried over sodium sulfate, and concentrated under reduced pressure. The crude product was purified by prep-TLC to give compound A38 (38 mg, 32%) as a white solid.

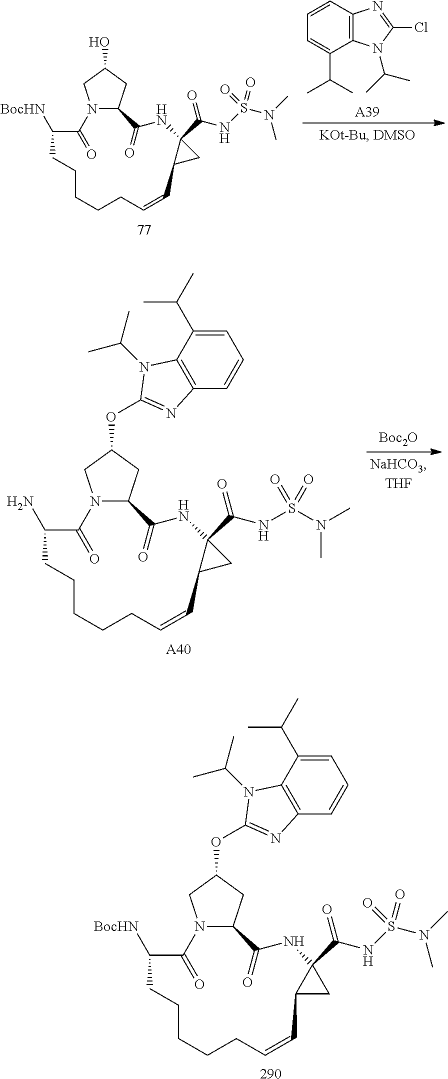

The same procedure for making compound 1223 as described in section 2.26 was used to prepare compound 290 (6.2 mg, 11%). MS (ESI) m/z (M+H)⁺ 772.4.

### 2.29 Synthesis of Compound 262 (2.8)

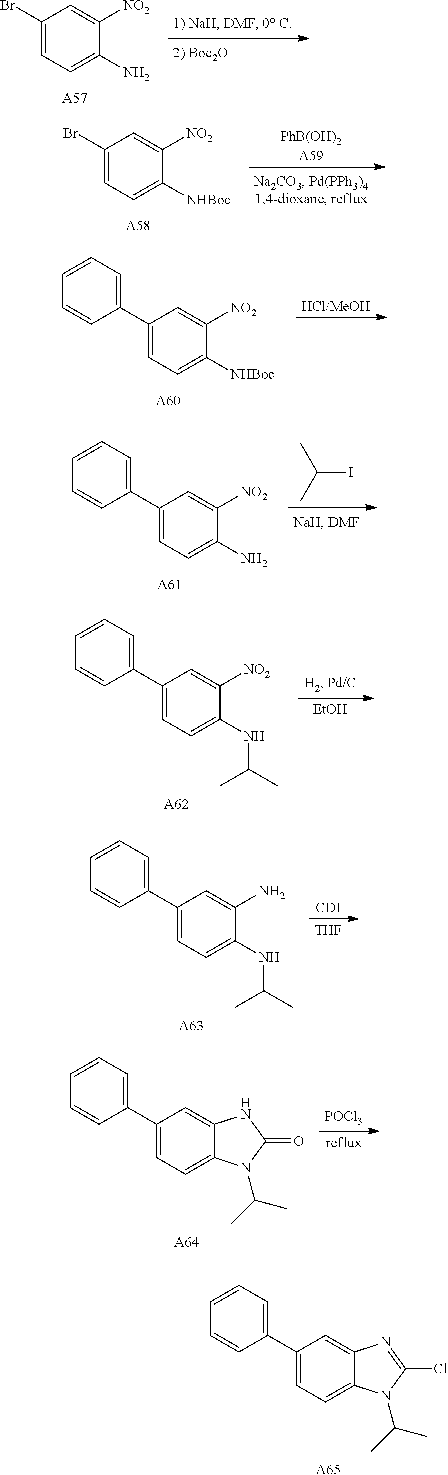

To a solution of compound A57 (5 g, 23.04 mmol) in DMF (25 mL) at 0° C. was added NaH (60%, 1.01 g, 25.3 mmol) portion-wise. After complete H₂ evolution, a solution of Boc₂O (5.53 g, 25.3 mmol) in 5 mL of DMF was added slowly into the reaction mixture over 30 min. The reaction was allowed to warm to room temperature, and stirred overnight. TLC showed the reaction completed. The reaction was quenched with water and the mixture was taken up in water and extracted with ethyl acetate (70 mL×3). the organic layers were combined, washed with brine, dried over anhydrous sodium sulfate, the solvent was removed under reduced pressure, the crude product was purified by silica gel column chromatography to afford compound A58 (3.2 g, 43.8%).

A flask was charged with compound A58 (3.2 g, 10.09 mmol), Na₂CO₃ (2.14 g, 20.19 mmol), compound A59 (2.7 g, 20.19 mmol) and Pd(PPh₃)₄ (2.33 g, 2.018 mmol). The flask was degassed with nitrogen for three times, then 1,4-dioxane (20 mL) and a drop of water were added. The resulting mixture was heated to reflux overnight under nitrogen protection. After completion of the reaction, the reaction was cooled to room temperature and the solvent was evaporated, the residue was diluted with ethyl acetate (200 mL). The solid was filtered off and the filtrate was concentrated in vacuo. The residue was purified by flash chromatography to afford compound A60 (1.5 g, 47.3%).

Compound A60 (1.5 g, 4.78 mmol) was dissolved in a solution of HCl in MeOH (4 M, 15 mL). The mixture was stirred at room temperature for 18 hrs. TLC analysis showed the reaction completed. The reaction mixture was concentrated under reduced pressure; the residue was taken up in water and basified with saturated aq. NaHCO₃, extracted with ethyl acetate (70 mL×3), the organic layers were combined, washed with brine, dried over anhydrous sodium sulfate and concentrated in vacuo to give the crude compound A61, which was used directly in the next step without further purification (1.0 g, 98%).

To a solution of compound A61 (490 mg, 2.29 mmol) in DMF (5 mL) was added NaH (60%, 110 mg, 2.75 mmol) in portions at 0° C. After stirring for 15 min, 2-iodopropane (389 mg, 2.29 mmol) was added thereto. The mixture was stirred at room temperature for 20 hrs. TLC analysis showed the reaction completed. The mixture was diluted with water, extracted with ethyl acetate (50 mL×3). The organic layers were combined, washed with brine, dried over anhydrous sodium sulfate, and concentrated in vacuo. The residue was purified by prep-TLC to give compound A62 (200 mg, 34.0%).

A mixture of compound A62 (200 mg, 1.17 mmol) and Pd/C (30 mg) in EtOH (10 mL) was degassed with hydrogen for three times, and then stirred at room temperature under a pressure of hydrogen atmosphere (30 psi) for 18 hrs. After the reaction completed, the mixture was filtered and the filtrate was concentrated in vacuo to afford crude compound A63, which was used directly in the next step without further purification (250 mg, 95%).

To a solution of compound A63 (250 mg, 1.1 mmol) in TRF (5 mL) was added CDI (361 mg, 2.2 mmol), the resulting mixture was stirred at room temperature overnight. The reaction was monitored with TLC. After completion of the reaction, the solvent was removed under reduced pressure, and the resulting mixture was purified by prep-TLC to give compound A64 as brown solid (200 mg, 72%).

A solution of compound A64 (120 mg, 0.47 mmol) in POCl₃ (3 mL) was heated to reflux for 16 hrs. TLC analysis showed the reaction completed. After cooling to r.t., the mixture was poured into ice water, neutralized with saturated aqueous NaHCO₃, and then extracted with ethyl acetate (20 mL×3), the organic layers were combined, washed with brine, dried over anhydrous sodium sulfate, and concentrated in vacuo to give crude compound A65 (120 mg, 94%), which was used directly in the next step without further purification.

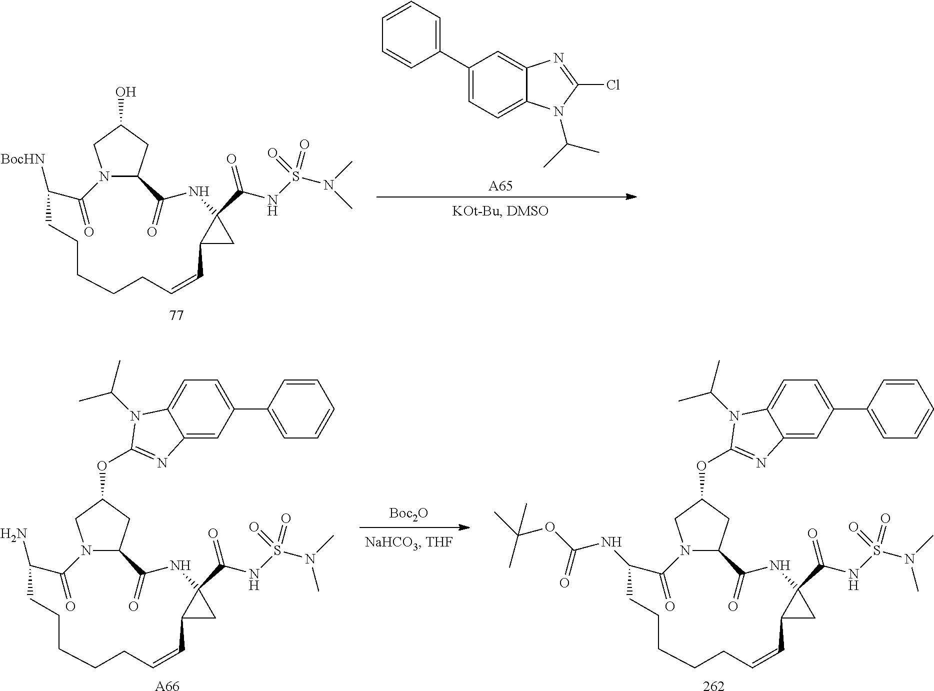

The same procedure for making compound 1223 as described in section 2.26 was used to prepare compound 262 (54.4 mg, 17.5%). MS (ESI) m/z (M+H)⁺ 806.5.

### 2.30 Synthesis of Compounds 263 and 264

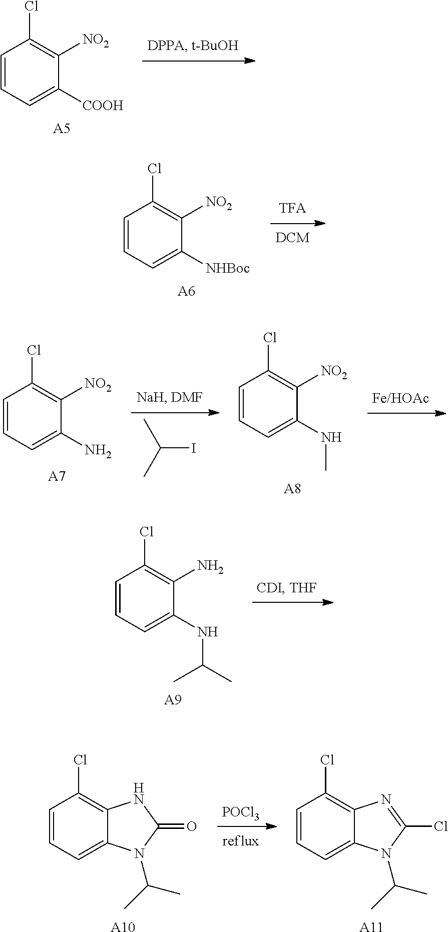

Compound A5 (4 g, 19.9 mmol) in t-BuOH (20 mL) containing Et₃N (2.9 mL, 20.9 mmol) was treated with DPPA (5.75 g, 20.9 mmol) and stirred at 100° C. overnight. After cooling to r.t., the mixture was poured into water and extracted with EtOAc (100 mL×3), the organic layer was combined and washed with brine, dried over anhydrous sodium sulfate, concentrated in vacuo. The residue was purified on silica gel column chromatography, eluting by petroleum ether and ethyl acetate (7:1), to afford compound A6 (5 g, 92%).

A flask was charged with compound A6 (5 g, 18 mmol), TFA (5 mL) and anhydrous CH₂Cl₂ (15 mL). The mixture was stirred at room temperature for 1 h. After the material was consumed, excess TFA was removed under reduced pressure. The residue was taken up in water and basified with NH₄OH. The aqueous layer was extracted with EtOAc (100 mL×3), the organic layer was combined and washed with brine, dried over anhydrous sodium sulfate, concentrated in vacuo to provide compound A7 (3 g, 95%).

A flask was charged with compound A7 (2 g, 11.6 mmol) and DMF (15 mL) and purged with nitrogen. To the resulting mixture was added NaH (60%, 0.93 g, 23.2 mmol) portion-wise at 0° C. After stirring for 30 min at 0° C., 2-iodopropane (3.9 g, 23.2 mmol) was added thereto dropwise. Then the mixture was allowed to warm to room temperature and stirred overnight. The mixture was quenched slowly by adding MeOH and then taken up in water. The aqueous layer was extracted with EtOAc (70 mL×3), the organic layer was combined and washed with brine, dried over anhydrous sodium sulfate, concentrated in vacuo to provide compound A8 (1.8 g, 72%), which was used directly in next step without further purification.

A round bottom flask was charged with compound A8 (1.3 g, 6.06 mmol); MeOH (20 mL) and HOAc (6 mL) was added to dissolve it. To the resulting mixture was added iron powder (1.35 g, 24.24 mmol) in portions at r.t. The reaction was monitored by TLC. After stirring for 1 h, the reaction was completed, the solvents were removed under reduced pressure, the residue was basified with saturated aq. NaHCO₃ to pH=9-10, EtOAc (150 mL) was added. The mixture was filtered, and the filtrate was washed with brine, dried over anhydrous Na₂SO₄, concentrated in vacuo to afford compound A9 as a brown oil (0.5 g, 45%).

A microwave tube was charged with compound A9 (550 mg, 3 mmol), CDI (0.97 g, 6 mmol) and anhydrous THF (5 mL), the reaction mixture was heated at 120° C. under microwave for 1 h. After cooling to r.t, the mixture was concentrated, the residue was purified on silica gel column chromatography (PE:EA=10:1) to afford compound A10 (200 mg, 32%).

The procedure for making compound A65 as described in section 2.28 was used to prepare compound A11 (40 mg, 91%).

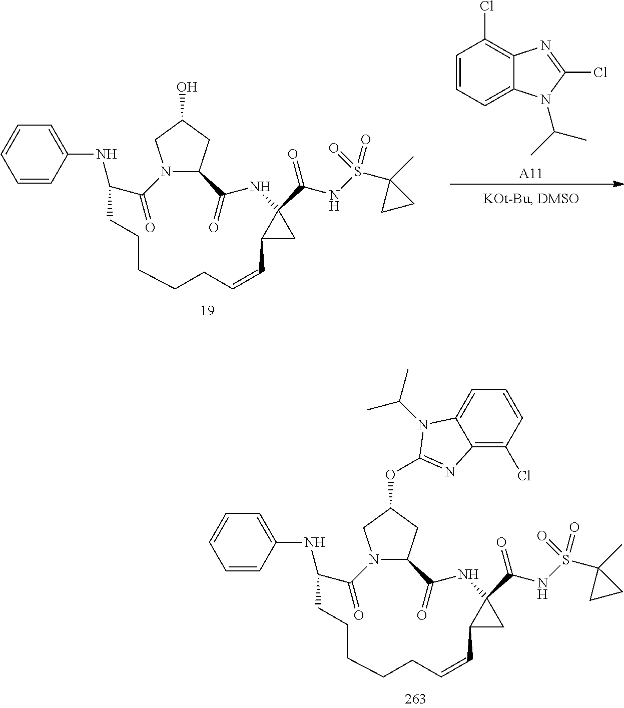

A flask was charged with compound 19 (56 mg, 0.1 mmol) and DMSO (1.5 mL), the solution was purged with nitrogen and then KOt-Bu (45 mg, 0.4 mmol) was added thereto. The mixture was stirred at room temperature for 1 hour. Then compound A11 (23 mg, 0.1 mmol) was added and the mixture was stirred for 12 hrs at room temperature. LCMS shows the reaction completed and the reaction was quenched by ice-water, acidified with aq. HCl (1 N) to pH=5-6, extracted with EtOAc (20 mL×3). The combined organic layers were washed with brine, dried over Na₂SO₄, concentrated in vacuo. The residue was purified with preparative HPLC to afford 263 as a light yellow solid (17 mg, 23%). MS (ESI) m/z (M+H)⁺ 751.3.

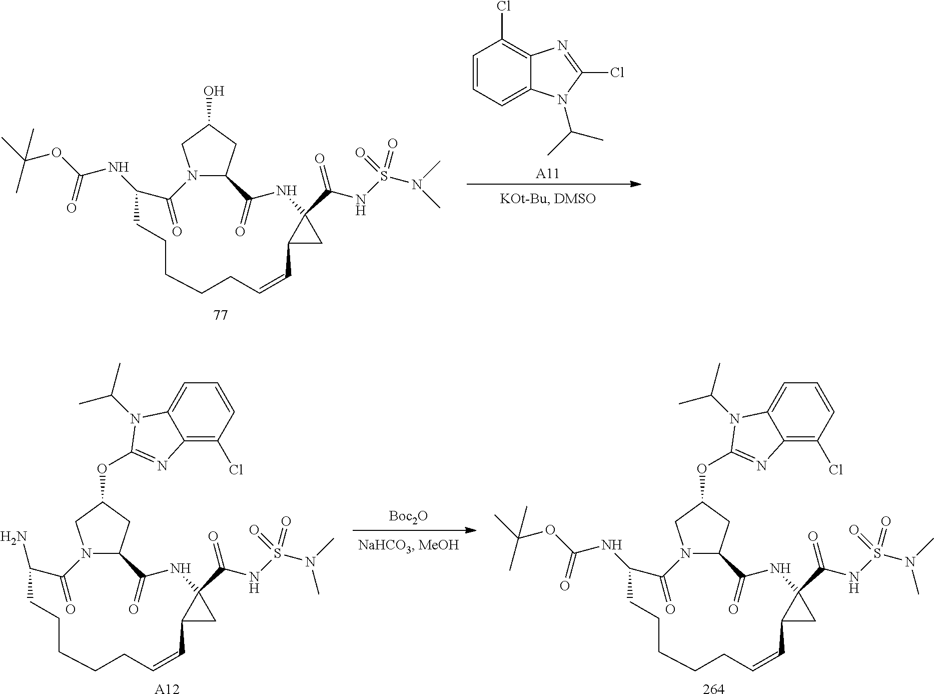

A flask was charged with compound 77 (57 mg, 0.1 mmol) and DMSO (1.5 mL), the solution was purged with nitrogen and then KOt-Bu (45 mg, 0.4 mmol) was added thereto. The mixture was stirred at room temperature for 30 min. Then compound A11 (23 mg, 0.1 mmol) was added and the mixture was stirred for 12 hrs at room temperature. LCMS shows the reaction completed and compound A12 was main product. The reaction was quenched by ice-water, neutralized with aq. HCl (1 M) to pH=6-7, the resulting mixture was used directly in next step.

To the resulting mixture above was added MeOH (2 mL), water (0.2 mL) and NaHCO₃ (10 mg, 0.12 mmol). Then Boc₂O (22 mg, 0.1 mmol) was added as well. The mixture was stirred at r.t. for 2 hrs. Then methanol was evaporated, the mixture was acidified with aq. HCl (1 N) to pH=5-6, extracted with EtOAc (15 mL×3). The combined organic layer was washed with brine, dried over Na₂SO₄, concentrated in vacuo. The residue was purified with preparative HPLC to afford compound 264 as an offwhite solid (21 mg, 28%). MS (ESI) m/z (M+H)⁺ 764.2.

### 2.31 Synthesis of Compound 265 (1.5)

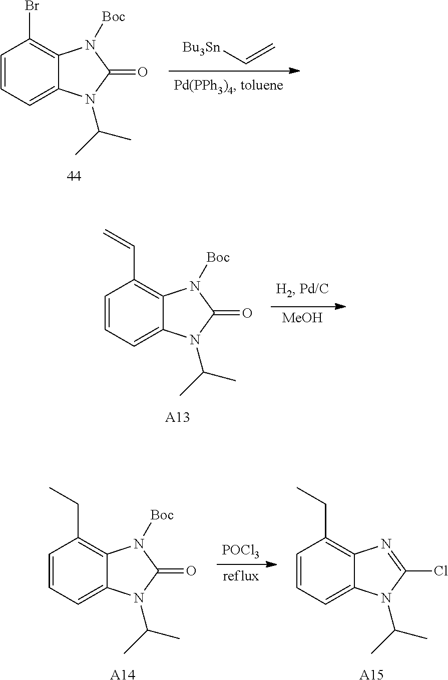

To a solution of compound 44 (0.12 g, 0.34 mmol) in toluene (20 mL) was added tributyl ethylene tin (0.32 g, 1.02 mmol), Pd(PPh₃)₄ (0.04 g, 0.034 mmol). The mixture was degassed with nitrogen three time and heated to reflux under nitrogen atmosphere for 12 hrs. The solvent was removed in vacuo, and the residue was purified by prep-TLC to give compound A13 (70 mg, 68%) as a light yellow oil. ¹H NMR: (400 MHz, CDCl₃): δ 7.18-7.14 (m, 2H), 7.02 (d, J=6.4 Hz, 1H), 6.88-6.74 (m, 1H), 5.15 (d, J=11.6 Hz, 1H), 4.72-4.62 (m, 1H), 1.66 (s, 9H), 1.55 (d, J=6.8 Hz, 6H).

An autoclave was charged with compound A13 (0.07 g, 2.23 mmol), MeOH (10 mL) and Pd/C (0.01 g) under nitrogen atmosphere. Then the mixture was degassed with hydrogen three times and stirred at r.t. under hydrogen atmosphere (30 psi) for 4 hrs. After the completion of reaction, the mixture was filtered, and the filtrate was concentrated to give compound A14 (70 mg, 99%) as a light yellow oil.

The procedure for making compound A65 as described in section 2.28 was used to prepare compound A15.

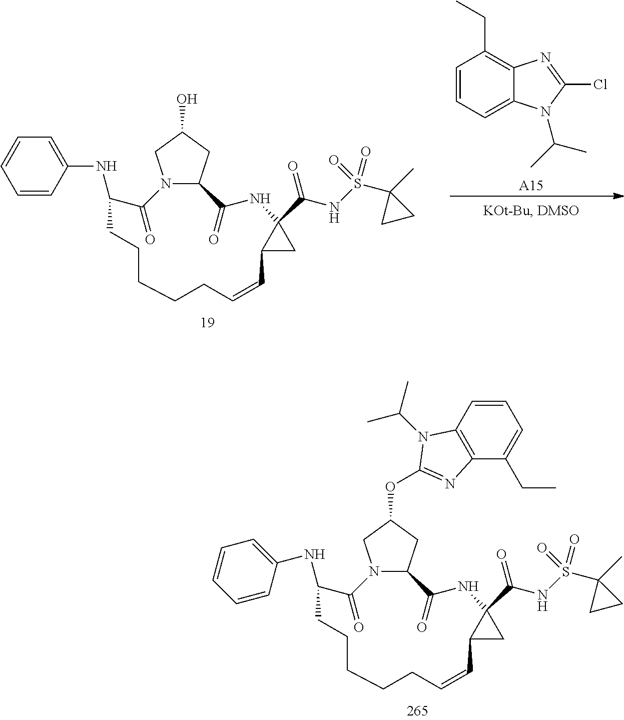

The same procedure for making compound 288 as described in section 2.22 was used to prepare compound 265 (9 mg, 17%). MS (ESI) m/z (M+H)⁺ 745.4.

### 2.32 Synthesis of Compound 266 (2.10)

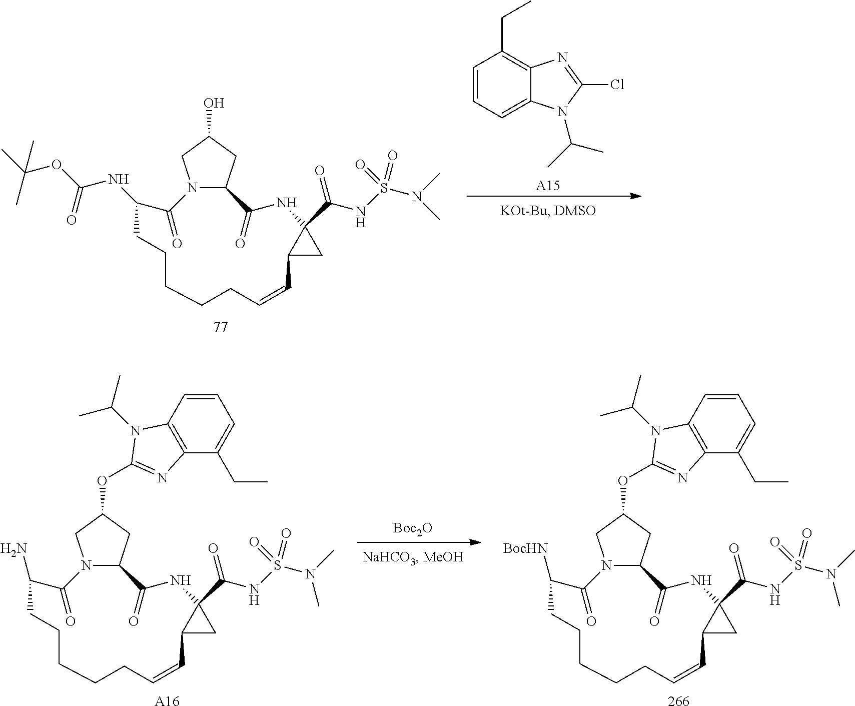

The same procedure for making compound 1223 as described in section 2.26 was used to prepare compound 266 (4.5 mg, 12%). MS (ESI) m/z (M+H)⁺ 758.4.

### 2.33 Synthesis of Compound 267-275 (1.6)

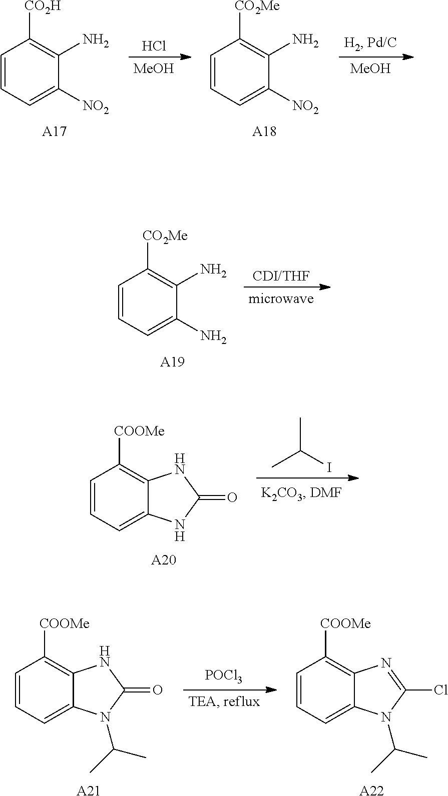

Compound A17 (20 g, 109.9 mmol) was dissolved in a solution of HCl/Methanol (4 M, 300 mL) and the mixture was refluxed for 12 hrs under nitrogen. After completion of the reaction, the mixture was concentrated under reduced pressure and then neutralized with saturated aq. NaHCO₃. The aqueous layer was extracted with EtOAc (200 mL×3), the organic layer was combined and washed with brine, dried over anhydrous sodium sulfate, concentrated in vacuo to give a crude product, it was purified on silica gel column chromatography (PE:EtOAc=30:1) to get compound A18 (20.1 g, 93%).

To a solution of compound A18 (20.0 g, 102 mmol) in MeOH (1 L) was added Pd/C (4 g). The reaction mixture was hydrogenated at r.t. under a pressure of 50 psi for 12 hrs. After completion of the reaction, the mixture was filtered and concentrated under reduced pressure to give a crude product A19 (14.0 g, 83%), which was used directly in next step without further purification.

To a solution of compound A19 (14.0 g, 84.3 mmol) in anhydrous TRF (400 mL) was added CDI (54.6 g, 337 mmol). The mixture was irradiated in microwave actor at 120° C. for 20 min. After that, the mixture was cooled to r.t. and neutralized with aq. HCl (0.1 M). The mixture was filtered and extracted with EtOAc (150 mL×3), the organic layer was combined and washed with brine, dried over anhydrous sodium sulfate, concentrated in vacuo to give a crude compound A20 (13.5 g, 83%), which was used directly in next step without further purification.

To a mixture of compound A20 (2.0 g, 11.4 mmol) and anhydrous K₂CO₃ (3.2 g, 22.7 mmol) in DMF (60 mL) was added 2-iodopropane (2.3 g, 13.6 mmol). The reaction mixture was stirred for 24 hrs at r.t. under nitrogen atmosphere. After completion of the reaction, the mixture was taken up in water and neutralized with aq. HCl (1 M). The mixture was extracted with EtOAc (70 mL×3), the organic layer was combined and washed with brine, dried over anhydrous sodium sulfate, concentrated in vacuo to give a crude product. It was purified on silica gel column chromatography (PE:EtOAc=20:1) to afford compound A21 (1.3 g, 49%). MS (ESI) m/z (M+H)⁺ 234.7. ¹H NMR (400 MHz, CDCl₃): δ 8.96 (s, 1H), 7.56 (d, J=8.0 Hz, 1H), 7.19 (d, J=2.4 Hz, 1H), 7.02 (t, J=8.0 Hz, 1H), 4.68-4.64 (m, 1H), 1.39 (d, J=6.4 Hz, 6H).

The procedure for making compound A65 as described in section 2.28 was used to prepare compound A22 (3 g, 41%). MS (ESI) m/z (M+H)⁺ 252.8.

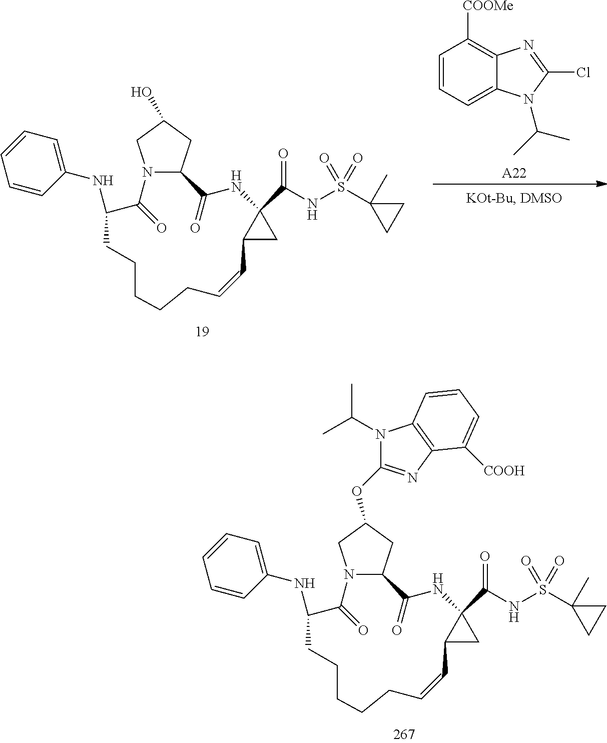

The procedure for making compound 288 as described in section 2.22 was used to prepare compound 267 (29 mg, 19%). MS (ESI) m/z (M+H)⁺ 761.5.

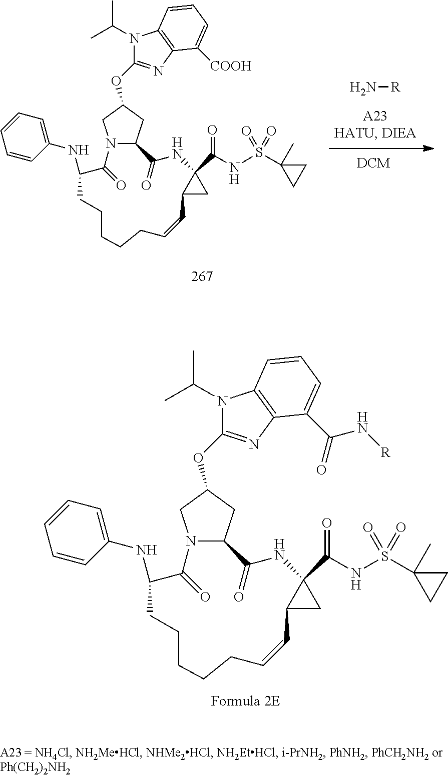

To a solution of compound 267 (1 eq.) in CH₂Cl₂ was added HATU (1.5 eq.), DIEA (4.0 eq.) and amine A23 (1.2 eq.). The reaction mixture was stirred at r.t. for 12 hrs. LCMS monitored the reaction, and then the mixture was concentrated in vacuo. The residue was purified with preparative HPLC to afford Formula 2E. The following compounds were prepared using this procedure.

TABLE 6
Compounds prepared according to the procedure for making Formula 2E.

| Compound | Structure | Yield |
|---|---|---|
| 268 | 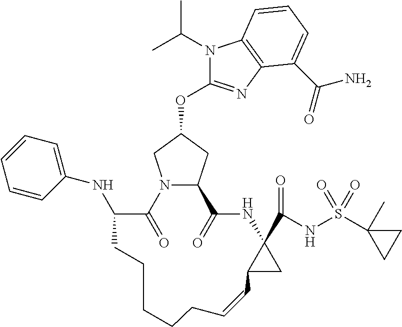 | 35 mg, 92%. MS (ESI) m/z (M + H)⁺ 760.5. |
| 269 | 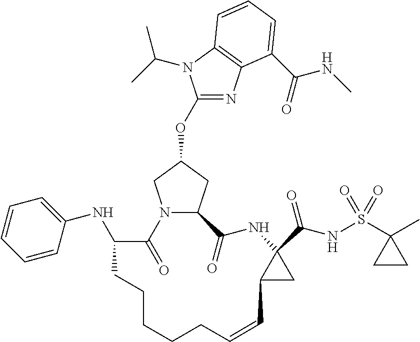 | 19 mg, 49%. MS (ESI) m/z (M + H)⁺ 774.3. |
| 270 | 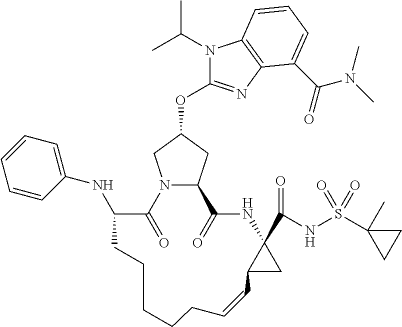 | 20 mg, 51%. MS (ESI) m/z (M + H)⁺ 788.6. |
| 271 | 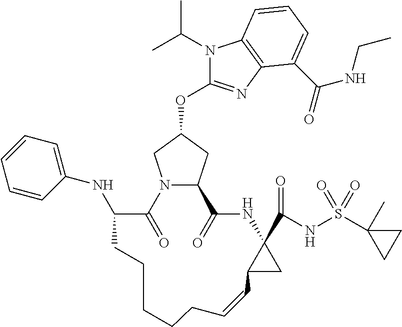 | 22.1 mg, 56%. MS (ESI) m/z (M + H)⁺ 788.6. |
| 272 | 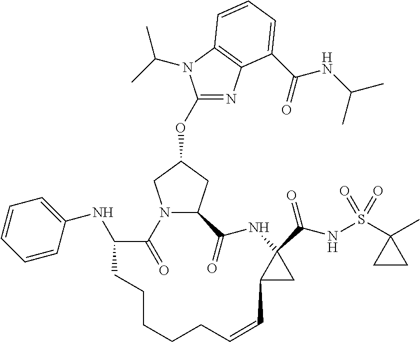 | 16 mg, 40%. MS (ESI) m/z (M + H)⁺ 802.3. |
| 273 | 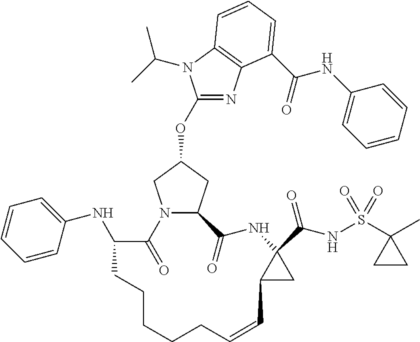 | 18 mg, 43%. MS (ESI) m/z (M + H)⁺ 836.3. |
| 274 | 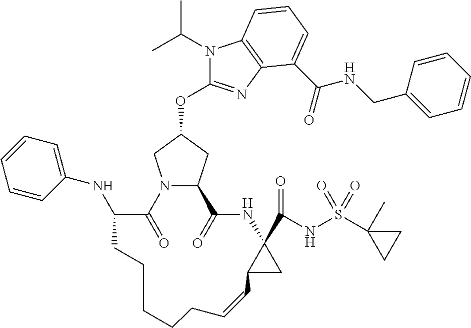 | 13.5 mg, 32%. MS (ESI) m/z (M + H)⁺ 850.3. |
| 275 | 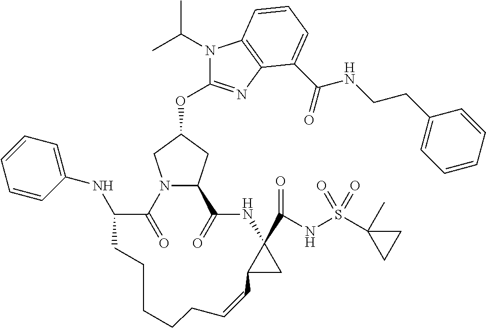 | 15.5 mg, 34%. MS (ESI) m/z (M + H)⁺ 864.3. |

### 2.34 Synthesis of Compound 276-284 (2.11)

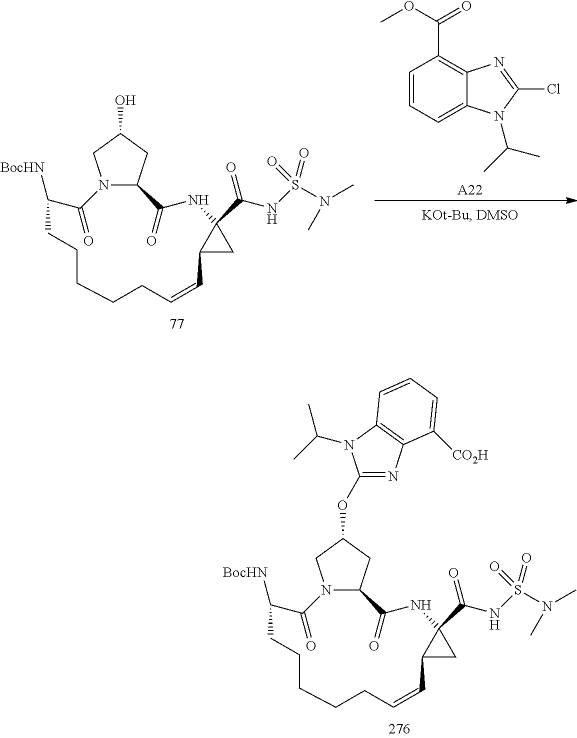

A flask was charged with compound 77 (114 mg, 0.2 mmol), KOt-Bu (101 mg, 0.9 mmol) and DMSO (3 mL). The resulting mixture was stirred at 0° C. for 30 min under nitrogen. Then compound A22 (60 mg, 0.24 mmol) was added thereto. The reaction mixture was stirred at r.t. for 16 hrs. The reaction was monitored with LCMS. LCMS showed the reaction completed and 276 was main product. The reaction mixture was quenched by ice-water, acidified with aq. HCl (1 M) to pH=6, extracted with EtOAc (30 mL×3). The combined organic layer was washed with brine, dried over Na₂SO₄, concentrated in vacuo. The residue was purified with preparative HPLC to afford compound 276 (31.5 mg, 20%). MS (ESI) m/z (M+H)⁺ 774.5.

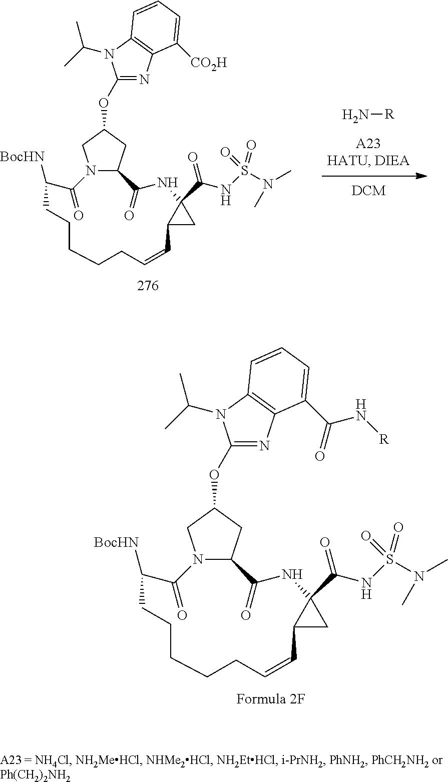

To a solution of compound 276 (1 eq.) in CH₂Cl₂ was added HATU (1.5 eq.), DIEA (4.0 eq.) and amine A23 (1.2 eq.). The reaction mixture was stirred at r.t. for 12 hrs. LCMS monitored the reaction. After completion of the reaction, the mixture was concentrated in vacuo. The residue was purified with preparative HPLC to afford Formula 2F. The following compounds were prepared using this procedure.

TABLE 7
Compounds prepared according to the procedure for making Formula 2F.

| Compound | Structure | Yield |
|---|---|---|
| 277 | 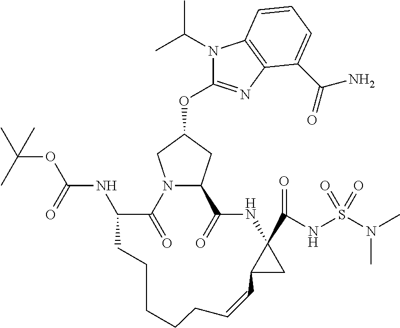 | 16 mg, 41%. MS (ESI) m/z (M + H)⁺ 773.6. |
| 278 | 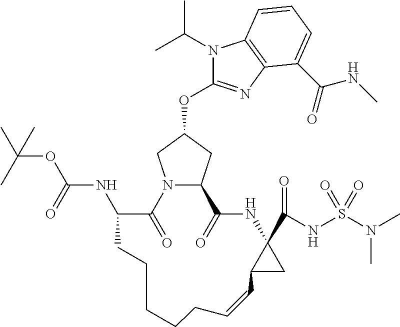 | 12.5 mg, 32%. MS (ESI) m/z (M + H)⁺ 787.6. |
| 279 | 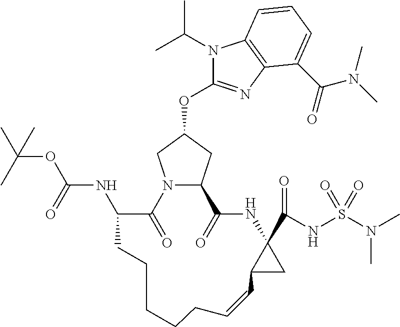 | 29.5 mg, 74%. MS (ESI) m/z (M + H)⁺ 801.6. |
| 280 | 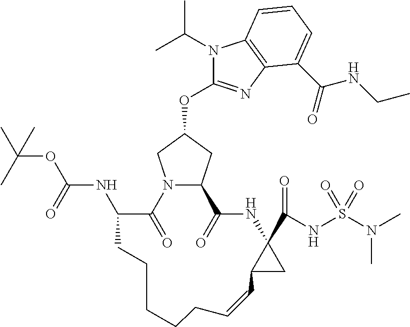 | 26 mg, 65%. MS (ESI) m/z (M + H)⁺ 801.6. |
| 281 | 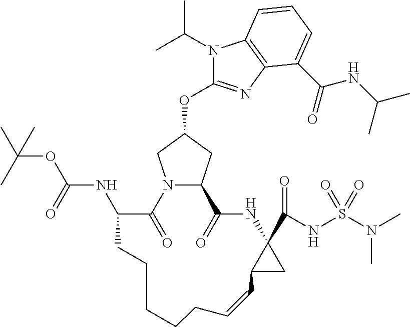 | 17.5 mg, 43%. MS (ESI) m/z (M + H)⁺ 815.6. |
| 282 | 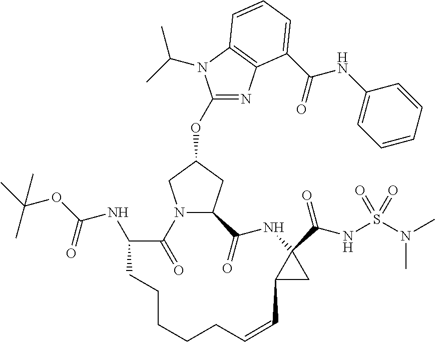 | 8 mg, 20%. MS (ESI) m/z (M + H)⁺ 849.5. |
| 283 | 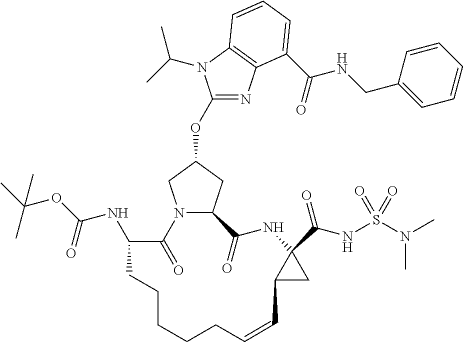 | 12.5 mg, 38%. MS (ESI) m/z (M + H) 863.4. |
| 284 | 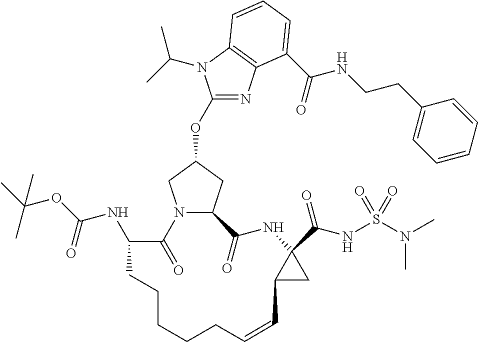 | 13 mg, 46%. MS (ESI) m/z (M + H)⁺ 877.5. |

### 2.35 Synthesis of Compound 285 (1.7)

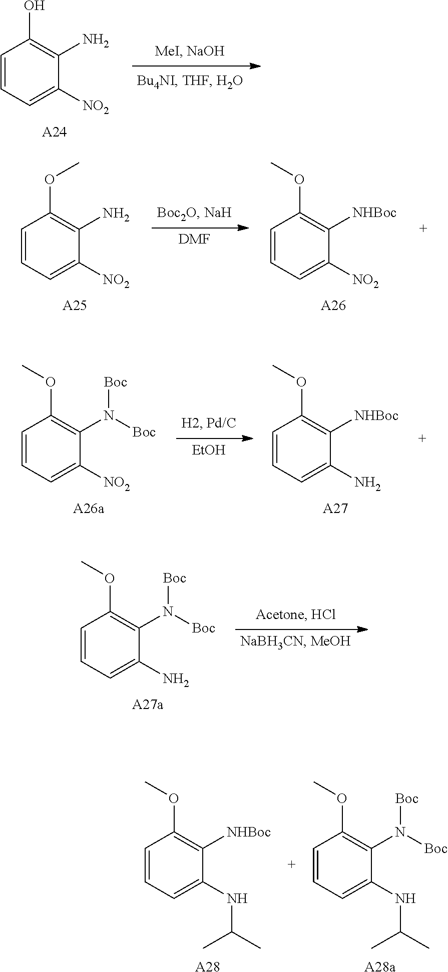

Tetrabutylaminonium iodide (0.4 g, 1.08 mmol, 0.4 eq.), sodium hydroxide (4 g, 100 mmol, 3.8 eq., in 4 mL of water) and methyl iodide (3.4 mL, 64.8 mmol, 2.5 eq.) were added into a solution of compound A24 (4 g, 25.95 mmol, 1 eq.) in 80 mL of TRF. The mixture was stirred at room temperature overnight. TLC analysis showed the reaction completed. The solvent was removed under vacuum, and the residue was diluted with water, extracted with ethyl acetate (50 mL×3). The organic layers were combined, washed with brine, dried over anhydrous sodium sulfate, the solvent was removed under reduced pressure to give the crude compound A25 (4.5 g, 103%), which was used directly in next step without further purification.

A flask was charged with NaH (60%, 0.71 g, 17.8 mmol) and 5 mL of DMF. A solution of compound A25 (2 g, 11.89 mmol) in 15 mL of DMF was added into the flask at 0° C. After stirring for 30 min, a solution of di-tert-butyl dicarbonate (2.59 g, 11.89 mmol) in 6 mL of DMF was added at 0° C. The mixture was allowed to warm to room temperature, and stirred overnight. TLC analysis showed the material was consumed. The mixture was diluted with water, extracted with ethyl acetate (50 mL×3). The organic layers were combined, washed with brine, dried over anhydrous sodium sulfate, concentrated in vacuo. The residue was purified by flash chromatography to give a mixture of compounds A26 and A26a (1.47 g).

Intermediates A27 and A27a were prepared using a procedure that is similar to the one described in section 2.32 for the preparation of compound A19.

To a solution of compounds A27 and A27a (600 mg) in MeOH (4.5 mL) was added acetone (0.37 mL, 5.0 mmol) and conc. HCl (0.27 mL) and the mixture was stirred for another hour at r.t. After that, sodium cyanoborohydride was added portion-wise at 0° C. and the mixture was stirred for 2 hrs at r.t. The reaction mixture was taken up into water and basified to pH=9 with saturated aq. NaHCO₃. The mixture was extracted with ethyl acetate (50 mL×3). The organic layers were combined, washed with brine, dried over anhydrous sodium sulfate, concentrated in vacuo. The residue was purified by flash chromatography to give compound 5 (180 mg) and 5a (270 mg) respectively.

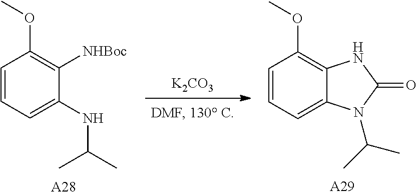

To a solution of compound A28 (100 mg, 0.356 mmol) in DMF (3 mL) was added K₂CO₃ (52 mg, 0.36 mmol). The reaction mixture was heated at 130° C. for 8 hrs. TLC analysis showed the reaction completed. The mixture was diluted with water, extracted with ethyl acetate (50 mL×3). The organic layers were combined, washed with brine, dried over anhydrous sodium sulfate, concentrated in vacuo. The residue was purified by prep-TLC to give compound A29 (50 mg, 68%).

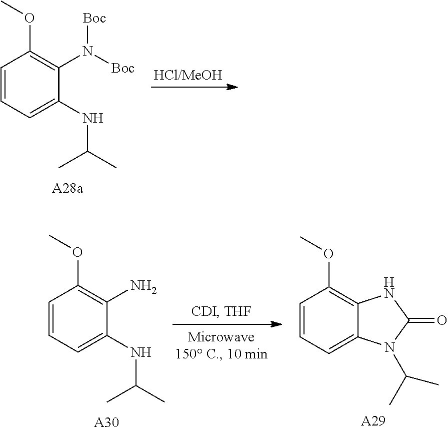

To a solution of free amine A30 (0.5 g, 3.62 mmol) in 8 mL of TRF was added CDI (2.36 g, 14.48 mmol). The reaction vessel was heated in microwave at 150° C. for 10 min. TLC analysis showed the reaction was completed. The reaction mixture was cooled to r.t., concentrated in vacuo. The residue was acidified with aq. HCl (1 M). Precipitate was formed and collected by filtration. The solid was compound A29 (90 mg, 65%).

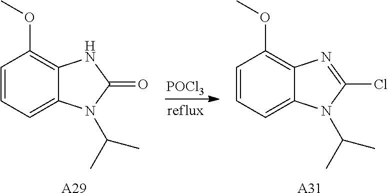

Compound A31 was prepared following the procedure for making compound A65 as described in section 2.28.

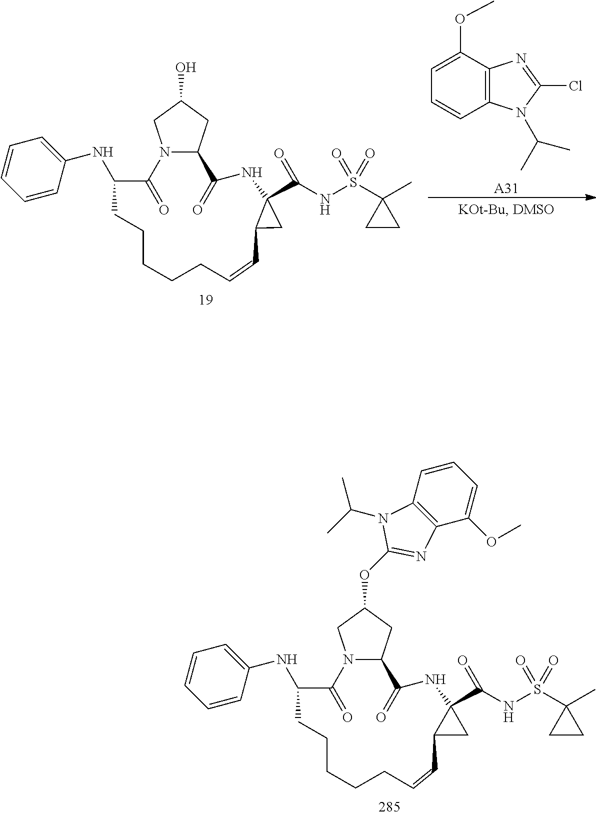

The same procedure for making compound 288 as described in section 2.22 was used to prepare compound 285 (15.2 mg, 23%). MS (ESI) m/z (M+H)⁺ 747.4.

### 2.36 Synthesis of Compound 286 (2.12)

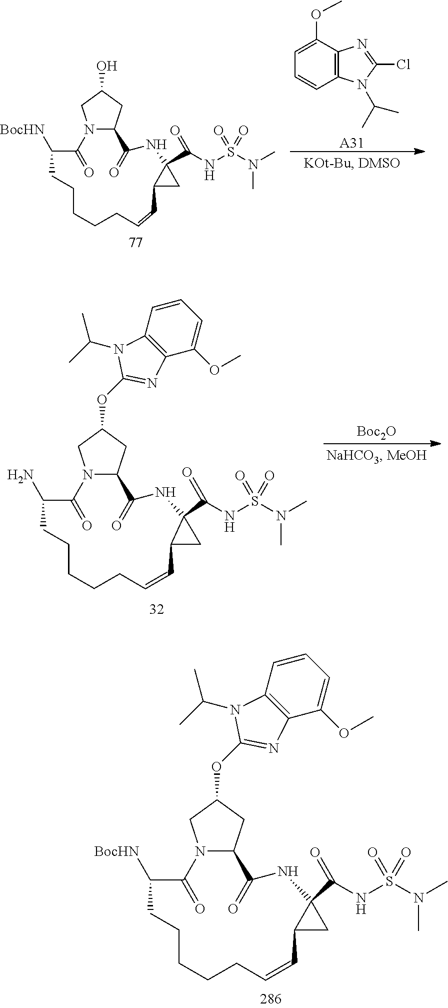

The same procedure for making compound 1217 as described in section 2.25 was used to prepare compound 286 (17 mg, 30%). MS (ESI) m/z (M+H)⁺ 760.3.

### 2.37 Synthesis of Compound 287 (1.8)

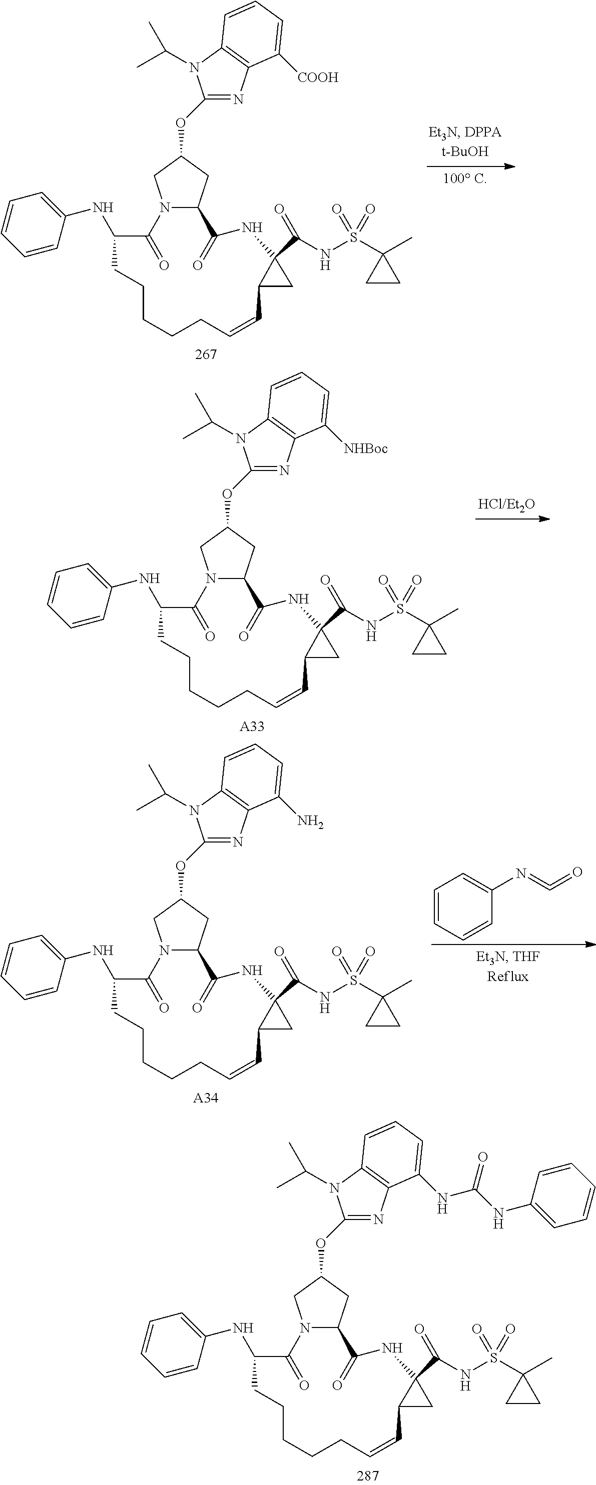

To a solution of compound 267 (40 mg, 0.053 mmol) in t-BuOH (2 mL) was added DPPA (15.2 mg, 0.055 mmol) and Et₃N (22 mg, 0.210 mmol). The mixture was heated at 100° C. for 4.5 hrs. LCMS showed the reaction completed. Upon cooling, the reaction mixture was diluted with ethyl acetate (100 mL), washed with aqueous citric acid (5%) and saturated aq. NaHCO₃, water and brine. The organic layer was dried over anhydrous sodium sulfate, filtered and concentrated under vacuum. The residue was purified by prep-TLC to give compound A33 (11 mg, 23%) as yellow solid.

A flask was charged with compound A33 (11 mg, 0.013 mmol) and a solution of HCl (gas) in Et₂O (3 mL). The reaction mixture was stirred for 2 hrs at room temperature. TLC analysis showed the reaction completed. The solvent was removed under vacuum to give compound A34 as light yellow solid, which was used directly in next step (10 mg, 99%).

To solution of compound A34 (15 mg, 0.0195 mmol) in anhydrous TRF (2 mL) was added 1-isocyanatobenzene and Et₃N (3 mg, 0.0293 mmol). The reaction mixture was heated to reflux for 8 hrs. The reaction was monitored with LCMS. After the reaction was completed, the mixture was concentrated in vacuo. The residue was purified by prep-HPLC to provide compound 287 (2.4 mg, 15%). MS (ESI) m/z (M+H)⁺ 851.4.

### 2.38 Synthesis of Compounds 292 and 293

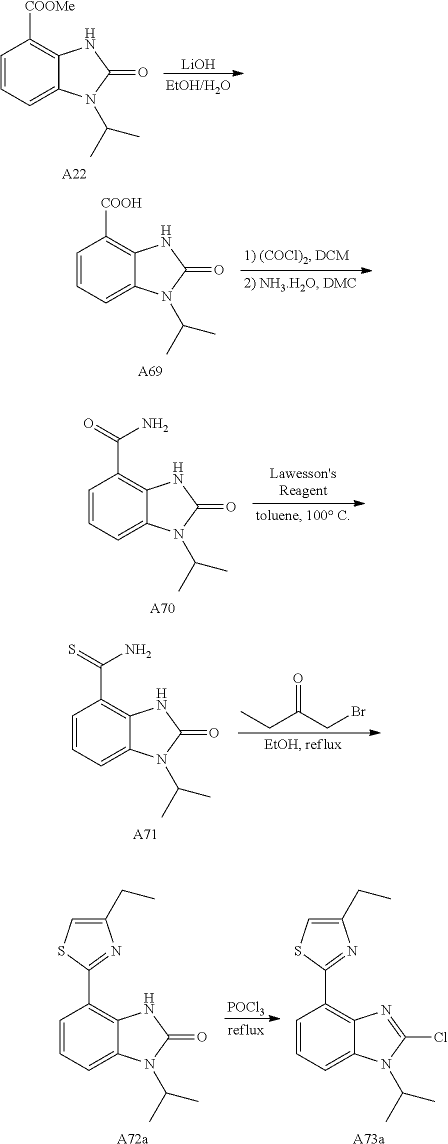

Preparation of compound A73a: Compound A22 (323 mg, 1.38 mmol) was dissolved in ethanol (4 mL) and water (2 mL), to the resulting solution was added LiOH (165 mg, 6.9 mmol). The reaction mixture was stirred at room temperature for 3 hrs. After completion of the reaction, the solvent was removed under reduced pressure, the aqueous layer was acidified to pH=4-5 with aq. HCl (1 M), extracted with EtOAc (40 mL×3), the combined organic layers was washed with brine, dried over sodium sulfate and concentrated in vacuo to give crude compound A69 (300 mg, 99%), which was used directly in the next step without further purification.

To a solution of compound A69 (200 mg, 0.91 mmol) in anhydrous CH₂Cl₂ (5 mL) was added oxalyl chloride (127 mg, 1 mmol) and DMF (one small drop) at 0° C. The mixture was stirred for 40 min at r.t. Then it was concentrated in vacuo. The residue was dissolved in anhydrous CH₂Cl₂ (5 mL), to the resulting solution was added ammonia (0.5 mL), and the reaction mixture was stirred at room temperature overnight. After the completion of the reaction, the mixture was filtered and concentrated to give compound A70 as white solid (130 mg, 65%).

A flask was charged with compound A70 (300 mg, 1.36 mmol), lawesson's reagent (278 mg, 0.682 mmol) and toluene (8 mL). The mixture was stirred at 100° C. for 3 hrs. The reaction was monitored with LCMS. After the completion of the reaction, the mixture was concentrated in vacuo and the residue was purified with prep-TLC to give compound A71 (200 mg, 62%).

A flask was charged with compound A71 (30 mg, 0.128 mmol), 1-bromobutan-2-one (20 mg, 0.128 mmol) and ethanol (2 mL). The reaction mixture was stirred at 100° C. for 1 h. The reaction was monitored with LCMS. After the completion of the reaction, the mixture was concentrated in vacuo and the residue was purified with prep-TLC to give product 6 (21 mg, 57%). ¹H NMR (400 MHz, CDCl₃): δ 9.90 (s, 1H), 7.41-7.39 (m, 1H), 7.14 (s, 1H), 7.08 (s, 1H), 6.86 (s, 1H), 4.77 (d, J=28 Hz, 1H), 2.86 (d, J=22 Hz, 2H), 1.58-1.55 (m, 6H), 1.35 (d, J=15.2 Hz, 6H).

A flask was charged with compound A72 (21 mg, 0.07 mmol) and POCl₃ (1 mL). The mixture was stirred at 100° C. for 5 hrs. After cooling to r.t., the mixture was poured into ice-water, extracted with EtOAc (30 mL×3), the combined organic layers was washed with saturated aq. NaHCO₃, dried over anhydrous sodium sulfate, and concentrated in vacuo to give compound A73a (22 mg, 100%).

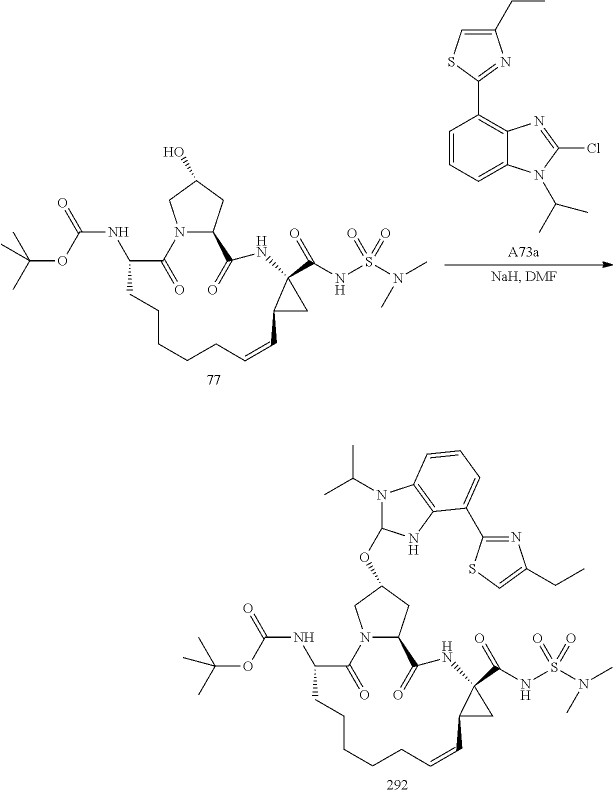

Preparation of compound 292: To a solution of compound 77 (50 mg, 0.087 mmol) in DMF (1 mL) was added NaH (60%, 25 mg, 0.632 mmol) at 0° C. The resulting mixture was stirred at 0° C. for 1 h, then compound A73a (29 mg, 0.097 mmol) was added thereto. The mixture was stirred at room temperature overnight. The reaction was monitored with LCMS. After the completion of the reaction, the mixture was quenched with ice-water, acidified to pH=5-6 with aq. HCl (1 M), extracted with EtOAc (30 mL×3), the combined organic layers was washed with brine, dried over sodium sulfate and concentrated in vacuo. The residue was purified by prep-HPLC to give compound 292 as white solid (20 mg, 27%). MS (ESI) m/z (M+H)⁺ 841.4.

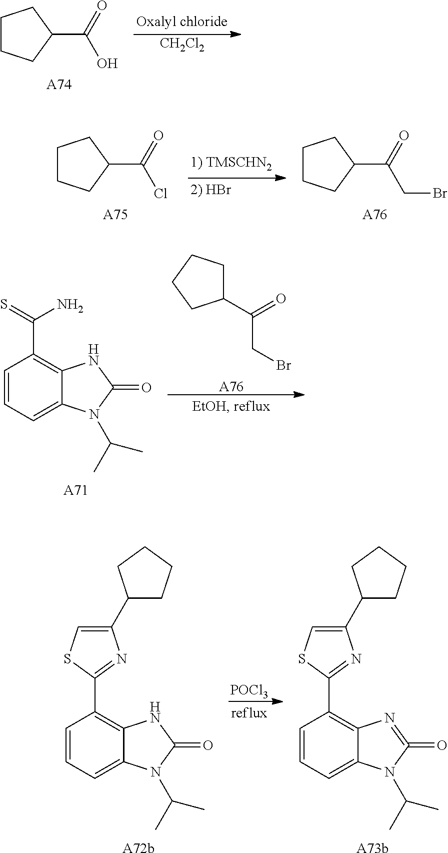

Preparation of compound A73b: To a solution of compound A74 (2.5 g, 21.9 mmol) in anhydrous CH₂Cl₂ (30 mL) was added oxalyl chloride (2.37 mL, 28 mmol) and one drop of DMF at 0° C. the resulting mixture was stirred for 2 hrs at room temperature. Then the mixture was concentrated in vacuo and the residue was dissolved in anhydrous TRF (20 mL) to yield compound A75. To the solution was added TMSCHN₂ (2.0 M in TRF, 52 mL, 105 mmol) dropwise. After the addition completed, the mixture was stirred at 0° C. for 1 h. After that a solution of HBr/AcOH (6.1 mL) was added. Stirring was continued for 30 min at 0° C. and then 12 hrs at room temperature. The mixture was poured into water and extracted with EtOAc (100 mL×3), the combined organic layers was washed with brine, dried over sodium sulfate and concentrated in vacuo to get crude A76 (4 g, 95%), which was used in the next step directly without further purification.

Compound A72b (150 mg, 61%, MS (ESI) m/z (M+H)⁺ 327.9) and A73b (150 mg, 95%, MS (ESI) m/z (M+H)⁺ 345.8) were made following the same procedure for making compounds A72a and A73a above, using compound A76.

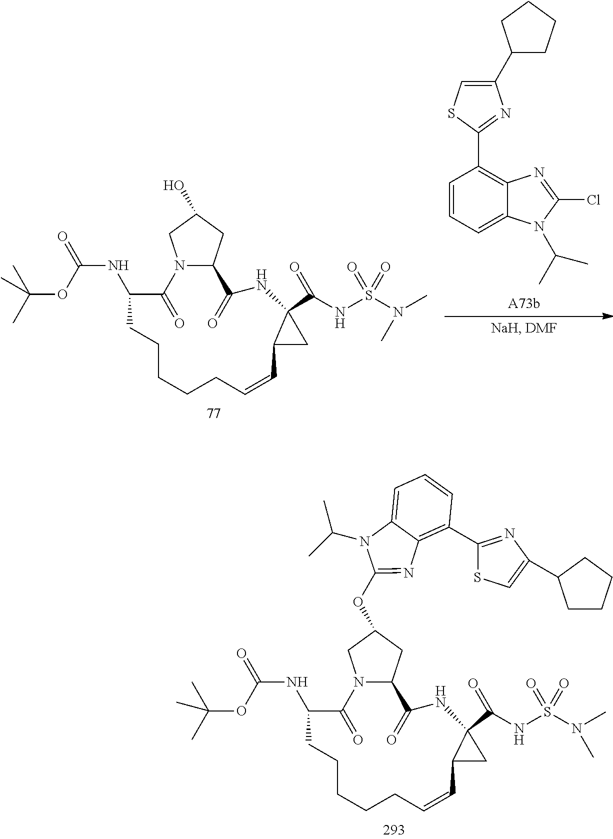

Compound 293 was prepared using the same procedure as for making compound 292, using compound A73b instead of compound A73a. Yielded 66 mg, 21%. MS (ESI) m/z (M+H)⁺ 881.3.

### 2.39 Synthesis of Compounds 294-299

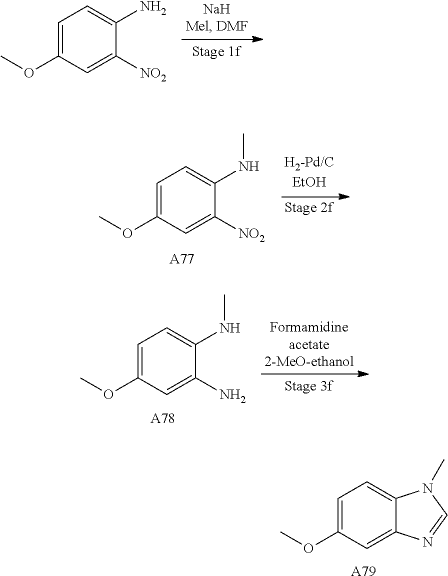

Preparation of precursors: 4-Methoxy-2-nitro-aniline (2.0 g, 11.9 mmol, 1.0 eq.) was dissolved in N,N-dimethylformamide (20 mL) and the solution cooled on top of an ice bath. Sodium hydride (60% dispersion in oil, 522 mg, 13.1 mmol, 1.1 eq.) was added portion wise to the cold solution. The reaction mixture was stirred at ambient temperature for a further 10 minutes. Methyliodide (1.11 mL, 17.8 mmol, 1.5 eq.) was added as a single portion. The reaction mixture was stirred at 35° C. for 90 minutes. Once the reaction was complete, the reaction mixture was partitioned between water (50 mL) and ethyl acetate (50 mL). The organic phase was collected and the aqueous phase further extracted with ethyl acetate (2×50 mL). The organic phases were combined, washed with water (2×50 mL) and brine (50 mL), dried over sodium sulfate, filtered and the solvent removed in vacuo to give 2.16 g (99% yield) of compound A77 as a red solid which was used in the next step without further purification. LC-MS: purity 93% (UV), t_{R} 1.84 min m/z [M+H]⁺ 182.95 (MET/CR/1278).

2-Methylamino-5-methoxy-nitrobenzene A77 (2.16 g, 11.9 mmol, 1.0 eq.) was dissolved in a mixture of ethanol (47 mL) and tetrahydrofuran (9 mL). 10% Pd/C (50% wet, 432 mg, 10 wt %) was added and the reaction flask flushed with nitrogen 3 times then placed under a hydrogen gas atmosphere for 12 hours. The reaction mixture was filtered on microfiber glass paper to and the solvent removed in vacuo to give 1.75 g (99% yield) of compound A78 as a red solid which was used in the next step without further purification. ¹H NMR (500 MHz, CDCl₃) δ ppm 6.62 (d, J=8.39 Hz, 1H) 6.35-6.42 (m, 2H) 3.75 (s, 3H) 3.05-3.58 (m, 2H) 2.83 (s, 3H) 1.27 (s, 1H). LC-MS: purity 99% (UV), t_{R} 0.49 min m/z [M+H]⁺ 153.00 (MET/CR/1278).

2-Methylamino-5-methoxy-aniline A78 (1.75 g, 11.8 mmol, 1.0 eq.) and formamidine acetate (2.47 g, 23.6 mmol, 2.0 eq.) were dissolved into 2-methoxy-ethanol (30 mL) and the reaction mixture heated under reflux for 15 hours. The solvent was removed in vacuo and the residue partitioned between dichloromethane (20 mL) and water (20 mL). The organic phase was collected and the aqueous phase further extracted with dichloromethane (3×20 mL). The organic phases were combined, dried over sodium sulfate, filtered and the solvent removed in vacuo to give 2.2 g (99% yield corrected for solvent) of compound A79 as a brown solid which contained 15% w/w of methoxy ethanol. ¹H NMR (250 MHz, CDCl₃) δ ppm 7.80-8.02 (m, 1H) 7.22-7.34 (m, 2H) 6.99 (dd, J=8.83, 2.28 Hz, 1H) 3.86-3.92 (m, 3H) 3.84 (s, 3H). LC-MS: purity 100% (UV), t_{R} 0.82 min m/z [M+H]⁺ 162.95 (MET/CR/1278).

1-Methyl-5-methoxy-benzimidazole A79 (1.0 g, 6.17 mmol, 1.0 eq.) was dissolved into 38% HBr in acetic acid (60 mL) and the solution was heated under reflux for 48 hours. The solvent was removed in vacuo and the residue purified by flash column chromatography (dichloromethane/methanol gradient) to give 146 mg (16% yield) of compound A80a (1-methyl-5-hydroxy-benzimidazole) as a red solid. ¹H NMR (500 MHz, MeOD) δ ppm 7.94 (s, 1H) 7.30 (d, J=8.70 Hz, 1H) 6.99 (s, 1H) 6.83 (d, J=8.70 Hz, 1H) 3.80 (s, 3H).

TABLE 8
Compounds A80b-A80g prepared using the method in section 2.38

| Compound | Structure | Yield |
|---|---|---|
| A80b | 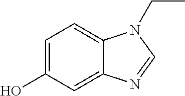 | 68 mg (8%) as a red solid. ¹H NMR (250 MHz, MeOD) δ ppm 8.12 (s, 1 H) 7.36 (d, J = 8.68 Hz, 1 H) 7.05 (d, J = 1.98 Hz, 1 H) 6.87 (dd, 1 H) 4.24 (q, J = 7.26 Hz, 2 H) 1.47 (t, J = 7.31 Hz, 3 H). |
| A80c | 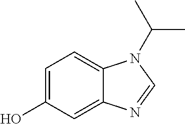 | 332 mg (35%) as a brown solid. ¹H NMR (500 MHz, MeOD) δ ppm 8.13 (s, 1 H) 7.39 (s, 1 H) 7.03 (d, J = 2.29 Hz, 1 H) 6.80-6.88 (m, 1 H) 4.69 (spt, J = 6.74 Hz, 1 H) 1.60 (d, 6 H). |
| A80d | 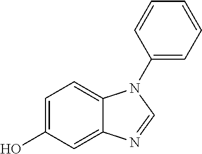 | 350 mg (36%) as a red solid. ¹H NMR (250 MHz, MeOD) δ ppm 8.31 (s, 1 H) 7.59-7.64 (m, 4 H) 7.50 (dd, J = 9.06, 4.95 Hz, 1 H) 7.42 (d, J = 8.83 Hz, 1 H) 7.12 (d, J = 2.28 Hz, 1 H) 6.90 (dd, J = 8.76, 2.36 Hz, 1 H). LC-MS: purity 100% (UV), t_{R} 1.25 min m/z [M + H]⁺ 210.90 (MET/CR/1278). |
| A80e | 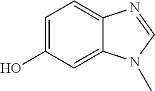 | 330 mg (50%) as a brown solid. ¹H NMR (500 MHz, MeOD) δ ppm 7.92 (s, 1 H) 7.44 (d, J = 8.70 Hz, 1 H) 6.85 (d, J = 2.29 Hz, 1 H) 6.79 (dd, J = 8.70, 2.29 Hz, 1 H) 3.78 (s, 3 H). |
| A80f | 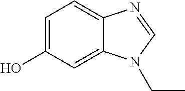 | 760 mg (82%) as a brown solid. ¹H NMR (500 MHz, MeOD) δ ppm 7.99 (s, 1 H) 7.45 (d, J = 8.70 Hz, 1 H) 6.88 (d, J = 2.29 Hz, 1 H) 6.79 (dd, J = 8.70, 2.29 Hz, 1 H) 4.63 (s, 0 H) 4.22 (q, J = 7.32 Hz, 2 H) 1.48 (t, J = 7.32 Hz, 3 H). LC-MS: purity 100% (ELS), t_{R} 0.52 min m/z [M + H]⁺ 162.95 (MET/CR/1278). |
| A80g | 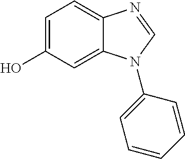 | 135 mg (48%) as a brown solid. ¹H NMR (500 MHz, CDCl₃) δ ppm 8.20 (br. s., 1 H) 7.70 (d, J = 8.70 Hz, 1 H) 7.55-7.61 (m, 2 H) 7.48-7.52 (m, 3 H) 7.03 (br. s., 1 H) 6.97 (d, J = 7.32 Hz, 1 H). LC-MS: purity 99% (ELS), t_{R} 1.11 min m/z [M + H]⁺ 210.95 (MET/CR/1278). |

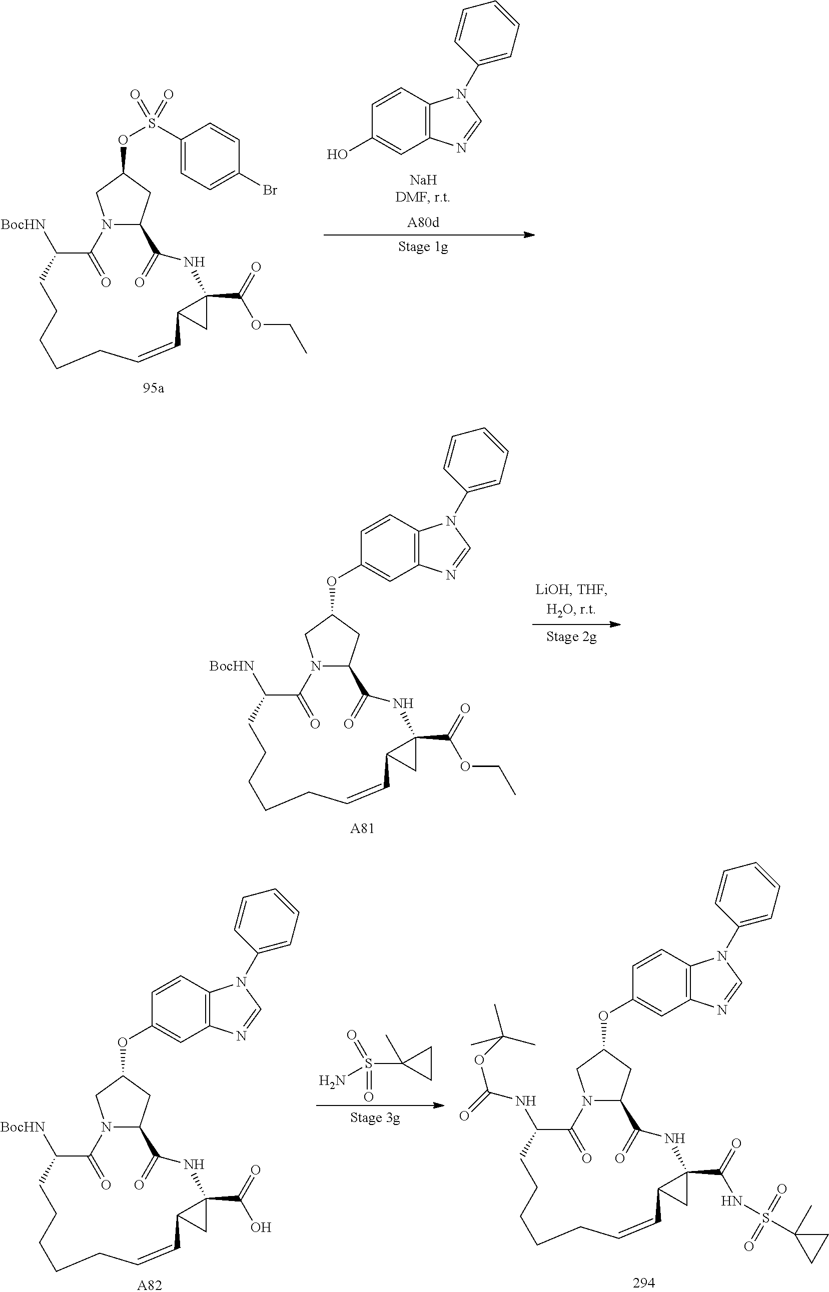

Preparation of compounds 294-299: Compound 294 was prepared using the same method described in section 7.2 below, with precursor compound A80d. Yielded 51 mg (14%) of compound 294 as a glassy solid after flash column chromatography. ¹H NMR (500 MHz, CDCl₃) δ ppm 9.89-10.45 (m, 1H) 8.40 (s, 1H) 7.58-7.66 (m, 2H) 7.49-7.57 (m, 3H) 7.40-7.48 (m, 2H) 7.29 (s, 1H) 7.01 (d, J=8.70 Hz, 1H) 5.68-5.78 (m, 1H) 5.34 (d, J=8.09 Hz, 1H) 5.18 (br. s., 1H) 5.02 (t, J=9.61 Hz, 1H) 4.64 (t, J=7.93 Hz, 1H) 4.25-4.45 (m, 2H) 3.92-4.01 (m, 1H) 2.57 (br. s., 2H) 2.28 (q, J=8.65 Hz, 1H) 1.58-2.00 (m, 5H) 1.50-1.54 (m, 1H) 1.49 (s, 3H) 1.38-1.46 (m, 2H) 1.36 (s, 9H) 1.28-1.33 (m, 2H) 1.19-1.27 (m, 4H) 0.79-0.86 (m, 2H). LC-MS: purity 100% (UV), t_{R} 4.51 min m/z [M+H]⁺ 775.30 (MET/CR/1416). Compounds 295-299 were prepared using the same method, using precursor compounds A80a, A80e, A80c, A70f and A80g, respectively.

TABLE 9
Compounds 296-299

| Compound | Structure | Yield |
|---|---|---|
| 295 | 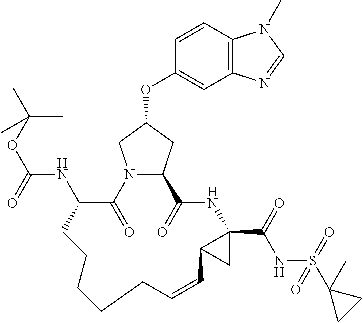 | 17 mg (15%) as a white solid after preparative HPLC. ¹H NMR (500 MHz, CDCl₃) δ ppm 10.28 (br. s., 1 H) 8.30 (d, J = 0.76 Hz, 1 H) 7.28-7.44 (m, 3 H) 7.00 (d, J = 8.85 Hz, 1 H) 5.71 (q, 1H) 5.25 (d, J = 7.93 Hz, 1 H) 5.14 (br. s., 1 H) 5.02 (t, J = 9.61 Hz, 1 H) 4.62 (t, J = 7.48 Hz, 1 H) 4.33-4.42 (m, 1 H) 4.27 (d, J = 10.83 Hz, 1 H) 3.95-4.02 (m, 1 H) 3.92 (s, 3 H) 2.44-2.60 (m, 3 H) 2.30 (q, J = 8.90 Hz, 1 H) 1.88-1.96 (m, 2 H) 1.74-1.85 (m, 2 H) 1.60-1.68 (m, 1 H) 1.50-1.57 (m, 2 H) 1.49 (s, 3 H) 1.39-1.47 (m, 3 H) 1.38 (s, 9H) 1.33-1.36 (m, 1 H) 1.25-1.33 (m, 2H) 0.77-0.87 (m, 2 H). LC-MS: purity 100% (UV), t_{R} 3.71 min m/z [M + H]⁺ 713.30 (MET/CR/1416). |
| 296 | 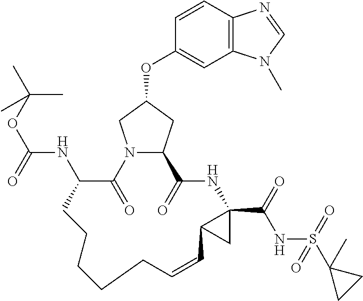 | 30.1 mg (31%) as a white solid. ¹H NMR (500 MHz, CDCl₃) δ ppm 7.84 (br. s., 1 H) 7.61-7.73 (m, 1 H) 7.52 (s, 1 H) 6.78-6.93 (m, 2 H) 5.71 (q, J = 9.05 Hz, 1 H) 5.30 (d, J = 7.93 Hz, 1 H) 5.11 (br. s., 1 H) 4.99 (t, J = 9.61 H z, 1 H) 4.57-4.73 (m, 2 H) 4.41 (t, J = 8.70 Hz, 1H) 4.31 (d, J = 11.29 Hz, 1 H) 3.93 (dd, J = 10.99, 3.66 Hz, 1 H) 3.77-3.86 (m, 3 H) 2.41-2.60 (m, 3 H) 2.32 (q, J = 8.85 Hz, 1 H) 1.70-1.99 (m, 4 H) 1.55-1.67 (m, 1 H) 1.49 (s, 3 H) 1.38 (s, 9 H) 1.17-1.54 (m, 8 H) 0.79-0.86 (m, 2 H). LC-MS: purity 96% (UV), t_{R} 3.71 min m/z [M + H]⁺ 713.30 (MET/CR/1416). |
| 297 | 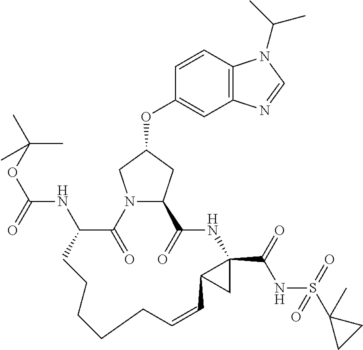 | 12.0 mg (20%) as a beige solid after flash column chromatography. ¹H NMR (500 MHz, CDCl₃) δ ppm 10.22 (br. s., 1 H) 8.58 (br. s., 1 H) 7.29-7.56 (m, 3 H) 7.02 (d, 1 H) 5.71 (q, 1 H) 5.21-5.28 (m, 1 H) 5.18 (br. s., 1 H) 5.03 (t, J = 9.77 Hz, 1 H) 4.68-4.78 (m, 1 H) 4.60-4.67 (m, 1H) 4.33-4.39 (m, 1 H) 4.28 (d, J = 10.99 Hz, 1 H) 4.03 (d, J = 11.14 Hz, 1 H) 2.51-2.61 (m, 3 H) 2.25-2.34 (m, 3 H) 2.12-2.19 (m, 2 H) 1.88-1.96 (m, 2 H) 1.73-1.77 (m, 1 H) 1.69 (d, J = 5.34 Hz, 6 H) 1.46-1.53 (m, 1 H) 1.49 (s, 3 H) 1.39-1.44 (m, 2 H) 1.27-1.41 (m, 9 H) 1.23-1.29 (m, 3 H) 0.78-0.87 (m, 2 H). LC-MS: purity 94% (UV), t_{R} 3.83 min m/z [M + H]⁺ 741.75 (MET/CR/1416). |
| 298 | 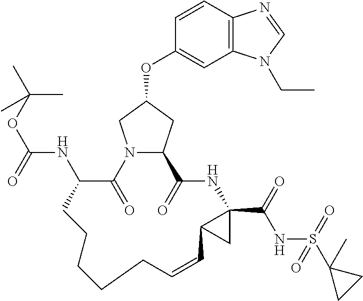 | 66 mg (47%) as a white solid after flash column chromatography. ¹H NMR (500 MHz, CDCl₃) δ ppm 9.82-10.69 (m, 1 H) 8.53 (br. s., 1 H) 7.89 (s, 1 H) 7.61-7.75 (m, 2 H) 6.71-6.95 (m, 2 H) 5.63-5.77 (m, 1 H) 5.35 (d, J = 8.39 Hz, 1 H) 5.05-5.23 (m, 1 H) 4.99 (t, J = 9.54 Hz, 1 H) 4.63 (t, J = 7.71 Hz, 1 H) 4.42 (t, J = 7.78 Hz, 1 H) 4.31 (d, J = 11.14 Hz, 1 H) 4.20 (q, J = 6.87 Hz, 2 H) 3.87-4.07 (m, 1 H) 2.43-2.60 (m, 3 H) 2.27-2.40 (m, 1 H) 1.84-1.98 (m, 2 H) 1.70-1.86 (m, 2 H) 1.57-1.66 (m, 1 H) 1.53 (t, J = 7.25 Hz, 3 H) 1.48 (s, 3 H) 1.46-1.51 (m, 2 H) 1.40-1.45 (m, 2 H) 1.34-1.40 (m, 1 H) 1.37 (s, 9 H) 1.24-1.34 (m, 2 H) 0.76-0.88 (m, 2 H). LC-MS: purity 99% (UV), t_{R} 3.78 min m/z [M + H]⁺ 727.75 (MET/CR/1416). |
| 299 | 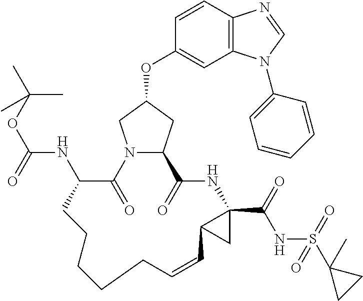 | 51 mg (50%) as a white glassy solid after flash column chromatography. ¹H NMR (500 MHz, CDCl₃) δ ppm 10.12 (br. s., 1 H) 8.10 (br. s., 1 H) 7.78 (d, J = 8.70 Hz, 1 H) 7.59-7.65 (m, 2 H) 7.52 (d, J = 7.78 Hz, 3 H) 6.96-7.05 (m, 2 H) 6.93 (d, J = 8.70 Hz, 1 H) 5.71 (q, 1 H) 5.21 (d, J = 8.39 Hz, 1 H) 5.06-5.16 (m, 1 H) 4.99 (t, J = 9.54 Hz, 1H) 4.54-4.66 (m, 1 H) 4.31-4.40 (m, 2 H) 3.90 (dd, J = 11.14, 3.36 Hz, 1 H) 2.45-2.58 (m, 3 H) 2.31 (q, J = 8.49 Hz, 1 H) 1.85-1.95 (m, 3 H) 1.64-1.85 (m, 1 H) 1.59 (t, J = 11.67 Hz, 2 H) 1.49-1.53 (m, 1 H) 1.48 (s, 3 H) 1.37-1.45 (m, 3 H) 1.34 (s, 9 H) 1.25-1.31 (m, 3 H) 0.82 (br. s., 2 H). LC-MS: purity 100% (UV), t_{R} 4.53 min m/z [M + H]⁺ 775.30 (MET/CR/1416). |

### 2.40 Synthesis of Compounds 1201-1207

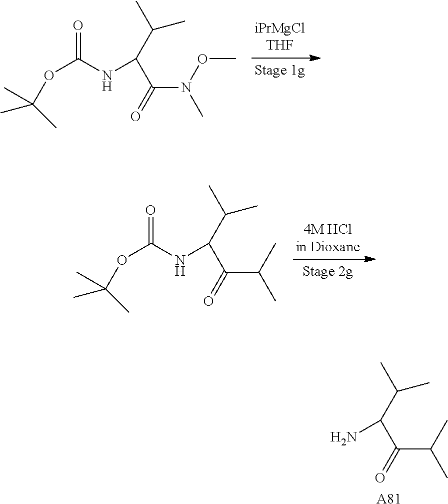

Preparation of precursors: Isopropyl magnesium chloride (2 M solution in diethyl ether, 24 mL, 49.9 mmol, 5.0 eq.) was added drop wise to the stirred solution of N-(tert-butoxycarbonyl)-L-valine N′-methoxy-N′-methylamide (2.6 g, 9.9 mmol, 1.0 eq.) in dry TRF (15 mL) at 0° C. The mixture was stirred for 2 hours at ambient temperature then carefully quenched with 1 M hydrochloric acid (3 mL) at 0° C. and extracted with diethyl ether (3×50 mL). The organic extracts were combined and washed with brine (100 mL), dried over magnesium sulphate, filtered and the solvent removed in vacuo. The residue was purified by flash column chromatography (ethyl acetate: heptanes gradient) to give 1.5 g (62% yield) of tert-Butyl [(3S)-2,5-dimethyl-4-oxohexan-3-yl]carbamate as a clear oil. ¹H NMR (500 MHz, CDCl₃) δ ppm 5.14 (d, J=8.39 Hz, 1H) 4.43 (dd, J=9.00, 4.27 Hz, 1H) 2.81 (spt, J=6.87 Hz, 1H) 2.09-2.22 (m, 1H) 1.44 (s, 9H) 1.14 (d, J=7.02 Hz, 3H) 1.09 (d, J=6.56 Hz, 3H) 1.01 (d, J=6.71 Hz, 3H) 0.78 (d, J=6.87 Hz, 3H). LC-MS: purity 64% (UV), t_{R} 2.15 min m/z [M+Na]⁺ 266.053 (MET/CR/1278).

tert-Butyl [(3S)-2,5-dimethyl-4-oxohexan-3-yl]carbamate (1.0 g, 4.1 mmol, 1.0 eq.) was dissolved in 4 M HCl in dioxane (5 mL) and the resulting solution heated at 40° C. for 1 hour. The solvent was removed in vacuo and the residue (S)-4-Amino-2,5-dimethyl-hexan-3-one hydrochloride salt A81 (off-white solid) was used directly in the next step without purification. LC-MS: purity 100% TIC (no UV or ELS response), t_{R} 0.57 min m/z [M+H]⁺ 143.95 (MET/CR/1278).

2-Chloro-benzimidazole building blocks preparation:

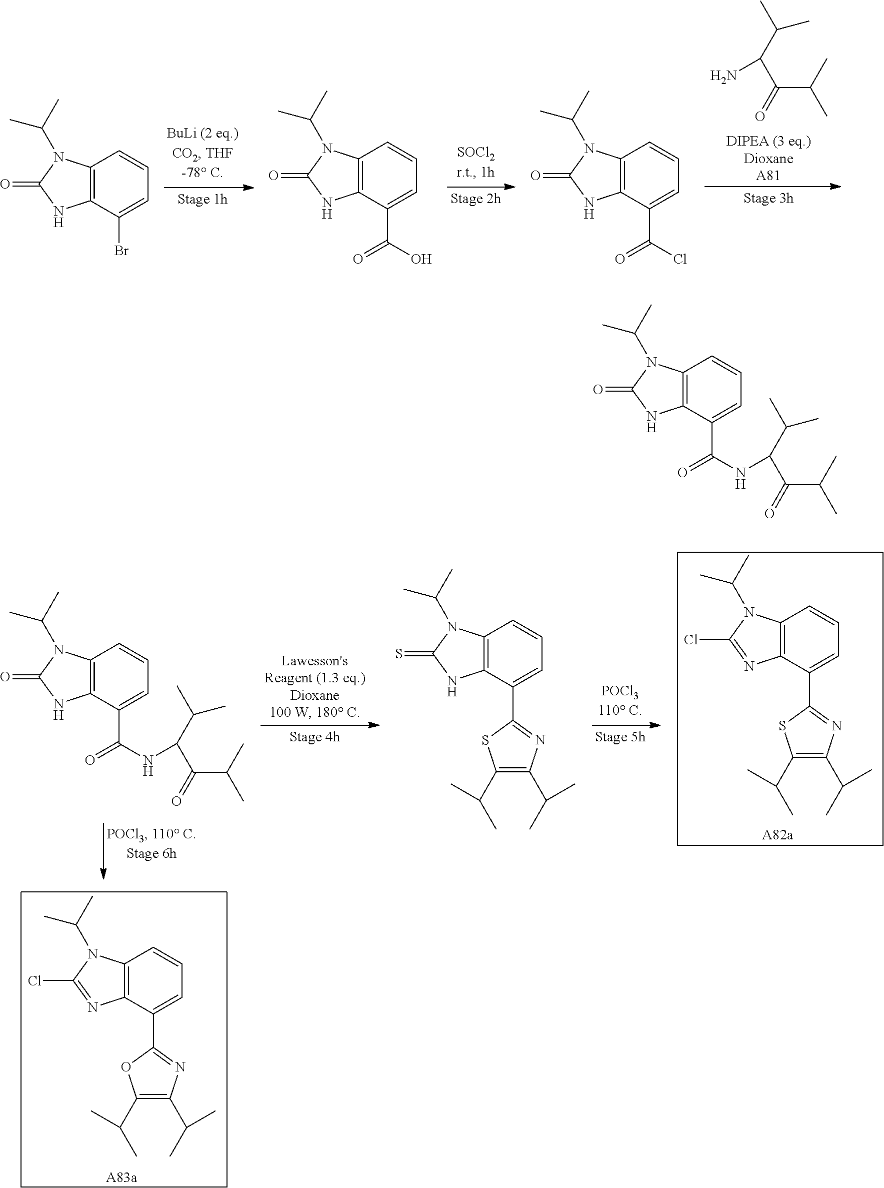

Stage 1h: A yellow solution of 4-bromo-1-isopropyl-1,3-dihydro-benzoimidazol-2-one (2.0 g, 7.8 mmol, 1.0 eq., see section 2.20 for preparation) in anhydrous tetrahydrofuran (10 mL) was added dropwise to a stirring solution of n-butyl lithium (2.5 M in hexanes, 7.8 mL, 2.5 eq.) in anhydrous tetrahydrofuran (10 mL) under nitrogen at −78° C. The resultant orange brown solution was slowly warmed to 0° C. over 20 minutes and anhydrous carbon dioxide gas was bubbled through the solution for 30 minutes. The resultant bright yellow suspension was then quenched with saturated ammonium chloride solution (40 mL). The aqueous layer was washed with ethyl acetate (2×40 mL), acidified to pH 2-3 with 1 M hydrochloric acid and extracted with ethyl acetate (3×60 mL). The organic extracts were combined, washed with brine, dried over magnesium sulfate and the solvent removed in vacuo to give 983 mg (57% yield) of 1-Isopropyl-2-oxo-2,3-dihydro-1H-benzoimidazole-4-carboxylic acid as an off-white solid which was used in the next step without further purification. ¹H NMR (250 MHz, CDCl₃) δ ppm 10.15 (br. s., 1H) 7.79 (dd, J=7.99, 0.84 Hz, 1H) 7.36 (d, J=7.92 Hz, 1H) 7.09-7.22 (m, 1H) 4.76 (d, J=7.01 Hz, 1H) 1.58 (d, J=7.01 Hz, 6H). LC-MS: purity 94% (UV), t_{R} 1.51 min m/z [M+H]⁺ 220.95 (MET/CR/1278).

Stage 2-3h: 1-Isopropyl-2-oxo-2,3-dihydro-1H-benzoimidazole-4-carboxylic acid (575 mg, 2.61 mmol, 1 eq.) was dissolved in thionyl chloride (6 mL) under nitrogen and the solution stirred for 1 hour at ambient temperature and the solvent removed in vacuo. The residue was dissolved in dry dioxane (5 mL) and diisopropylethylamine (1.36 mL, 7.83 mmol, 3 eq.) was added drop wise. (S)-4-Amino-2,5-dimethyl-hexan-3-one hydrochloride salt (738 mg, 4.10 mmol 1.5 eq.) as a suspension in dioxane (10 mL) was added portion wise and stirring was continued at ambient temperature for a further for 4 hours. The solution was diluted with water (50 mL) and extracted with ethyl acetate (3×100 mL). The organic extracts were combined and washed with water (50 mL) and brine (50 mL), dried over magnesium sulphate, filtered and the solvent removed in vacuo to give 900 mg (99% yield) of 1-isopropyl-2-oxo-2,3-dihydro-1H-benzoimidazole-4-carboxylic acid-((S)-1-isopropyl-3-methyl-2-oxo-butyl)-amide as a light oil which was used in the next step without further purification. ¹H NMR (500 MHz, CDCl₃) δ ppm 9.32 (br. s., 1H) 7.26-7.30 (m, 1 H) 7.24 (d, J=7.93 Hz, 1H) 7.09 (t, J=7.93 Hz, 1H) 6.91 (d, J=8.54 Hz, 1H) 5.03 (dd, J=8.62, 4.20 Hz, 1H) 4.74 (spt, J=7.04 Hz, 1H) 2.89 (spt, J=6.84 Hz, 1H) 2.24-2.36 (m, 1 H) 1.54 (d, J=6.87 Hz, 6H) 1.18 (d, J=7.02 Hz, 3H) 1.14 (d, J=6.71 Hz, 3H) 1.07 (d, J=6.71 Hz, 3H) 0.87 (d, J=6.87 Hz, 3H). LC-MS: purity 95% (UV), t_{R} 2.00 min m/z [M+H]⁺ 346.55 (MET/CR/1278).

Stage 4h: 1-Isopropyl-2-oxo-2,3-dihydro-1H-benzoimidazole-4-carboxylic acid-((S)-1-isopropyl-3-methyl-2-oxo-butyl)-amide (651 mg, 1.88 mmol, 1.0 eq.), Lawesson's reagent (994 mg, 2.45 mmol, 1.3 eq.) and dry dioxane (7 mL) were charged into a microwave tube. The reaction mixture was then irradiated in a focus microwave apparatus (100 W, 180° C.) for 30 minutes. The solvent was removed in vacuo and the residue purified by flash column chromatography (ethyl acetate: heptanes gradient) to give 449 mg (66% yield) of 4-(4,5-Diisopropyl-thiazol-2-yl)-1-isopropyl-1,3-dihydro-benzoimidazole-2-thione as a light yellow solid. ¹H NMR (500 MHz, CDCl₃) δ ppm 11.22 (br. s., 1H) 7.46 (d, J=7.78 Hz, 1H) 7.37 (d, J=8.09 Hz, 1H) 7.18 (t, J=7.93 Hz, 1H) 5.57-5.72 (m, 1H) 3.33 (spt, J=6.76 Hz, 1H) 3.16 (spt, J=6.97 Hz, 1H) 1.61 (d, J=7.02 Hz, 6H) 1.38 (d, J=6.87 Hz, 6H) 1.35 (d, J=6.87 Hz, 6H). LC-MS: purity 92% (UV), t_{R} 2.51 min m/z [M+H]⁺ 360.45 (MET/CR/1278).

Stage 5h: 4-(4,5-Diisopropyl-thiazol-2-yl)-1-isopropyl-1,3-dihydro-benzoimidazole-2-thione (449 mg, 1.25 mmol, 1.0 eq.) was dissolved in phosphorous oxychloride (5 mL) and the reaction mixture heated at 110° C. for 18 hours. The solvent was removed in vacuo and the residue partitioned between water (5 mL) and ethyl acetate (5 mL). The mixture was neutralized with saturated sodium hydrogen carbonate (pH=7) and the aqueous layer further extracted with ethyl acetate (3×10 mL). The combined organic layers were washed with water (25 mL) and brine (25 mL), dried over magnesium sulfate, filtered and the solvent removed in vacuo to give 350 mg (99% yield) of 1-Isopropyl-2-chloro-4-(4,5-diisopropyl-thiazol-2-yl)-benzoimidazole A82a as a yellow solid which was used in the next step without further purification. ¹H NMR (500 MHz, CDCl₃) δ ppm 8.25 (br. s., 1H) 7.49 (d, J=8.09 Hz, 1H) 7.33 (t, J=7.93 Hz, 1H) 4.96 (spt, J=6.97 Hz, 1H) 3.34 (spt, J=6.76 Hz, 1H) 3.09-3.24 (m, 1H) 1.68 (d, J=7.02 Hz, 6H) 1.31-1.42 (m, 12H). LC-MS: purity 96% (UV), t_{R} 2.45 min m/z [M+H]⁺ 363.00 (MET/CR/1278).

Stage 6h: 1-Isopropyl-2-oxo-2,3-dihydro-1H-benzoimidazole-4-carboxylic acid-((S)-1-isopropyl-3-methyl-2-oxo-butyl)-amide (308 mg, 0.88 mmol, 1.0 eq.) was dissolved in phosphorous oxychloride (3 mL) and the solution heated at 110° C. under nitrogen for 3 hours. The resultant brown solution was cooled to room temperature and the solvent removed in vacuo. The brown oil was dissolved in dichloromethane (3 mL) and distilled water (3 mL) added. The pH of the aqueous layer was adjusted to pH 7-8 using saturated sodium hydrogen carbonate. The organic layer was washed with brine, dried over magnesium sulfate and the solvent removed in vacuo to afford 298 mg (98% yield) of 1-Isopropyl-2-chloro-4-(4,5-Diisopropyl-oxazol-2-yl)-benzoimidazole A83a as a brown oil which was used in the next step without further purification. ¹H NMR (500 MHz, CDCl₃) δ ppm 7.86 (d, J=7.63 Hz, 1H) 7.52 (d, J=8.24 Hz, 1H) 7.30 (t, J=7.93 Hz, 1H) 4.90-4.98 (m, 1H) 3.13-3.21 (m, 1H) 2.96-3.07 (m, 1H) 1.65 (d, J=7.02 Hz, 5H) 1.35 (d, J=7.02 Hz, 6H) 1.32 (d, J=7.02 Hz, 6H). LC-MS: purity 98% (UV), t_{R} 2.57 min m/z [M+H]⁺ 346.40, 348.05 (MET/CR/1278).

TABLE 10
Precursor compounds A82b-A82d and A83b-A83d.

| Compound | Structure | Yield |
|---|---|---|
| A82b | 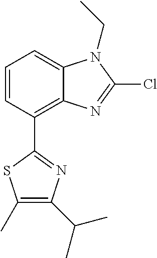 | 32 mg (99%) as a brown solid after work-up. ¹H NMR (250 MHz, CDCl₃) δ ppm 8.33 (d, J = 7.61 Hz, 1 H) 7.74 (d, J = 8.07 Hz, 1 H) 7.61 (t, J = 7.92 Hz, 1 H) 4.42 (q, J = 7.26 Hz, 2 H) 3.44 (spt, J = 7.03 Hz, 1 H) 2.60 (s, 3 H) 1.41-1.59 (m, 9 H). LC-MS: purity 79% (UV), t_{R} 2.25 min m/z [M + H]⁺ 320.00 (MET/CR/1278). |
| A82c | 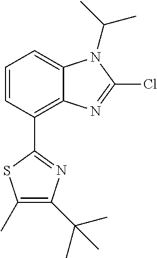 | 160 mg (96%) as a beige solid after work-up. ¹H NMR (500 MHz, CDCl₃) δ ppm 8.20 (d, J = 7.78 Hz, 1 H) 7.47 (d, J = 8.09 Hz, 1 H) 7.32 (t, J = 7.93 Hz, 1 H) 4.94 (spt, J = 6.99 Hz, 1 H) 2.60 (s, 3 H) 1.67 (d, J = 7.02 Hz, 6 H) 1.49 (s, 9 H). LC-MS: purity 80% (UV), t_{R} 2.87 min m/z [M + H]⁺ 348.40 (MET/CR/1278). |
| A82d | 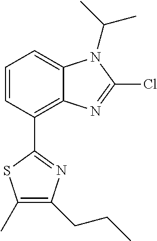 | 163 mg (85%) as a brown solid after work-up. ¹H NMR (500 MHz, CDCl₃) δ ppm 8.14 (d, J = 7.78 Hz, 1 H) 7.47 (d, J = 8.24 Hz, 1 H) 7.30 (t, J = 8.01 Hz, 1 H) 4.92 (spt, J = 6.99 Hz, 1 H) 2.73 (t, J = 7.55 Hz, 2 H) 2.43 (s, 3 H) 1.77 (sxt, J = 7.45 Hz, 2 H) 1.64 (d, J = 7.17 Hz, 6 H) 0.97 (t, J = 7.40 Hz, 3 H). LC-MS: purity 88% (UV), t_{R} 2.22 min m/z [M + H]⁺ 334.40 (MET/CR/1278). |
| A83b | 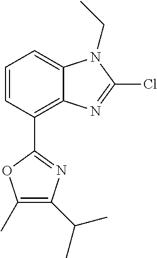 | 40 mg (100%) as a beige solid after work-up. ¹H NMR (250 MHz, MeOD) δ ppm 7.35 (d, J = 7.61 Hz, 1 H) 7.25 (d, J = 8.07 Hz, 1 H) 6.86 (t, J = 7.77 Hz, 1 H) 3.69 (q, J = 6.85 Hz, 2 H) 2.40-2.47 (m, 1 H) 1.74 (s, 3 H) 0.64 (t, J = 7.01 Hz, 3 H) 0.56 (d, J = 7.01 Hz, 6 H). LC-MS: purity 93% (UV), t_{R} 2.19 min m/z [M + H]⁺ 304.05 (MET/CR/1278). |
| A83c | 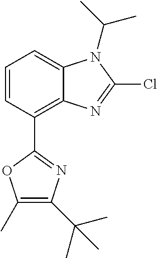 | 102 mg (100%) as a beige solid after work-up. ¹H NMR (500 MHz, CDCl₃) δ ppm 8.12 (br. s., 1 H) 7.58 (d, J = 8.09 Hz, 1 H) 7.35 (t, J = 8.01 Hz, 1 H) 4.96 (spt, J = 7.02 Hz, 1 H) 2.58 (s, 3 H) 1.67 (d, J = 7.02 Hz, 6 H) 1.43 (s, 9 H). LC-MS: purity 95% (UV), t_{R} 2.46 min m/z [M + H]⁺ 332.45 (MET/CR/1278). |
| A83d | 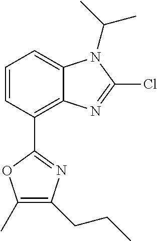 | 103 mg (100%) as a beige solid after work-up. ¹H NMR (500 MHz, CDCl₃) δ ppm 8.13 (br. s., 1 H) 7.62 (d, J = 8.09 Hz, 1 H) 7.38 (t, J = 7.93 Hz, 1 H) 4.98 (spt, J = 6.99 Hz, 1 H) 2.60 (t, J = 7.17 Hz, 2 H) 2.46 (s, 3 H) 1.77 (sxt, J = 7.42 Hz, 2 H) 1.69 (d, J = 7.02 Hz, 6 H) 0.98 (t, J = 7.40 Hz, 3 H). LC-MS: purity 100% (UV), t_{R} 2.26 min m/z [M + H]⁺ 318.10 (MET/CR/1278). |

Preparation of compounds 1201-1217:

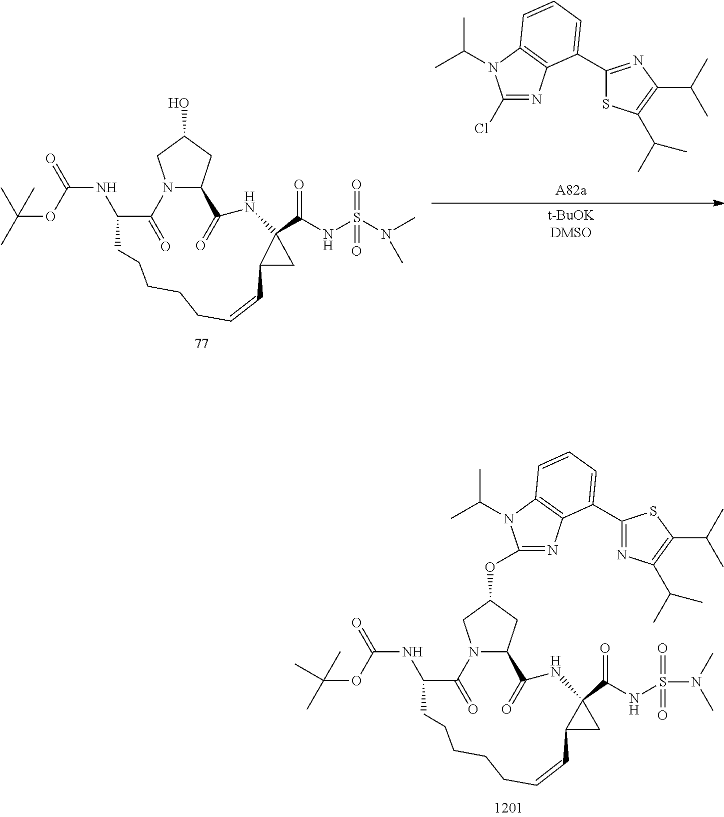

The hydroxyproline macrocycle 77 (92 mg, 0.161 mmol, 1.1 eq.), 1-isopropyl-2-Chloro-4-(4,5-diisopropyl-thiazol-2-yl)-benzimidazole A82a (53 mg, 0.146 mmol, 1.0 eq.) and anhydrous dimethylsulfoxide (1 mL) were charged into a 7 mL vial. Potassium tert-butoxide (66 mg, 0.585 mg, 4.0 eq.) was added in a single portion and the reaction mixture stirred at ambient temperature for 1 hour. The reaction mixture was diluted with water (4 mL) and extracted with ethyl acetate (5×4 mL). The combined organic extracts were washed with water (5×4 mL), dried over magnesium sulfate, filtered and the solvent removed in vacuo. The residue was purified by preparative HPLC to give 28 mg (21% yield) of compound 1201 as an off-white solid. ¹H NMR (500 MHz, CDCl₃) δ ppm 9.67-10.31 (m, 1H) 7.88-8.40 (m, 1H) 7.12-7.27 (m, 1H) 6.53-6.89 (m, 1H) 6.01 (br. s., 1 H) 5.76 (q, J=8.95 Hz, 1H) 4.95-5.10 (m, 2H) 4.51-4.82 (m, 3H) 4.25-4.39 (m, 1H) 3.98-4.15 (m, 1H) 3.28-3.45 (m, 1H) 3.08-3.29 (m, 1H) 2.88 (s, 6H) 2.81-2.87 (m, 1 H) 2.72-2.81 (m, 1H) 2.49-2.66 (m, 1H) 2.18-2.31 (m, 1H) 1.84-2.09 (m, 3H) 1.66-1.85 (m, 2H) 1.58-1.67 (m, 2H) 1.54 (d, J=6.87 Hz, 6H) 1.43-1.52 (m, 4H) 1.39 (d, J=6.56 Hz, 12H) 1.36 (s, 9H) 1.10-1.24 (m, 1H). LC-MS: purity 97% (UV), t_{R} 5.03 min m/z [M+H]⁺ 897.38 (MET/CR/1426).

TABLE 11
Compounds 1202-1217.

| Compound | Structure | Yield |
|---|---|---|
| 1202A | 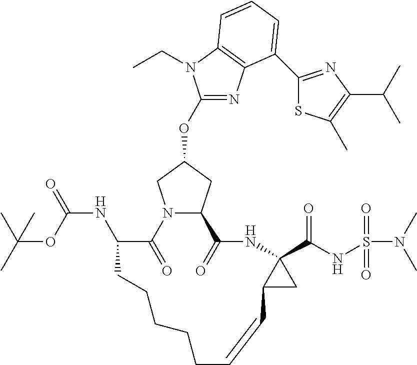 | 3.4 mg (5.1%) as a white solid after preparative HPLC. ¹H NMR (500 MHz, CDCl₃) δ ppm 10.01 (br. s., 1 H) 8.14 (br. s., 1 H) 7.27-7.29 (m, 1 H) 7.20-7.25 (m, 1H) 7.13-7.19 (m, 1 H) 6.69 (br. s., 1 H) 5.95 (br. s., 1 H) 5.71-5.81 (m, 1 H) 4.98-5.08 (m, 2 H) 4.61 (t, J = 7.96 Hz, 1 H) 4.55 (d, J = 12.14 Hz, 1 H) 4.24-4.34 (m, 1 H) 4.09-4.17 (m, 1 H) 3.97-4.08 (m, 2 H) 3.17 (br. s., 1 H) 2.89 (s, 6 H) 2.71-2.85 (m, 2 H) 2.53-2.64 (m, 1 H) 2.48 (s, 3 H) 2.18-2.27 (m, 1 H) 1.86-1.96 (m, 2 H) 1.75-1.85 (m, 2 H) 1.59-1.62 (m, 1 H) 1.47 (br. s., 5 H) 1.37 (d, J = 3.78 Hz, 3 H) 1.36 (d, J = 4.10 Hz, 6 H) 1.34 (br. s., 9 H). LC-MS: purity 100% (UV), t_{R} 5.79 min m/z [M + H]⁺ 855.10 (MET/CR/1416). |
| 1202B | 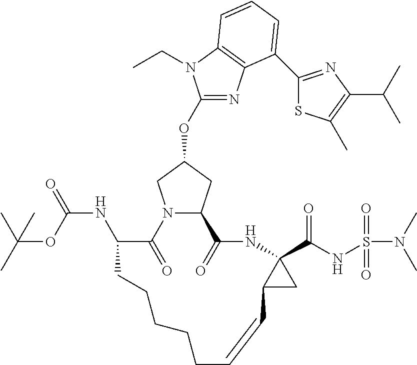 | 26 mg (41%) as an off-white solid after preparative HPLC. ¹H NMR (500 MHz, CDCl₃) δ ppm 10.04 (br. s., 1 H) 8.16 (br. s., 1 H) 7.27 (s, 3 H) 7.11-7.26 (m, 2 H) 6.72 (br. s., 1 H) 5.85-6.05 (m, 1 H) 5.68-5.82 (m, 1 H) 4.90-5.14 (m, 2 H) 4.43-4.72 (m, 2 H) 4.22-4.36 (m, 1 H) 4.09-4.18 (m, 1H) 3.96-4.09 (m, 2 H) 2.89 (s, 6 H) 2.71-2.85 (m, 2 H) 2.53-2.64 (m, 1 H) 2.50 (br. s., 3 H) 2.16-2.27 (m, 1 H) 1.71-1.98 (m, 4 H) 1.55-1.69 (m, 1 H) 1.35-1.54 (m, 11 H) 1.34 (s, 9 H) 1.14-1.32 (m, 3 H). LC-MS: purity 100% (UV), t_{R} 4.72 min m/z [M + H]⁺ 855.39 (MET/CR/1426). |
| 1203 | 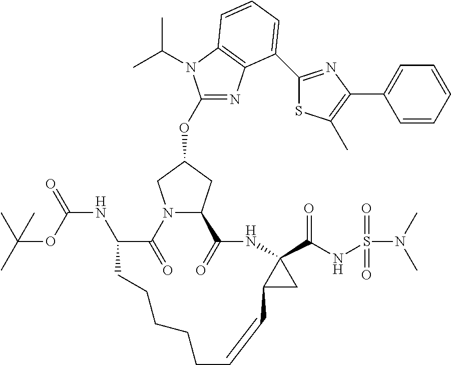 | 4.5 mg (10%) as a white solid after column chromatography. ¹H NMR (500 MHz, CDCl₃) δ ppm 9.89 (br. s., 1 H) 7.21 (dd, J = 8.62, 3.89 Hz, 1 H) 6.97 (dd, J = 10.83, 9.00 Hz, 1 H) 6.74 (br. s., 1 H) 5.98 (br. s., 1 H) 5.70-5.80 (m, 1 H) 4.98-5.08 (m, 2 H) 4.45-4.62 (m, 3 H) 4.24-4.33 (m, 1 H) 3.98 (dd, J = 11.67, 2.67 Hz, 1 H) 2.94-3.03 (m, 1 H) 2.87 (s, 6 H) 2.73-2.83 (m, 1 H) 2.64-2.73 (m, 1 H) 2.51-2.63 (m, 1 H) 2.41 (s, 3 H) 2.24 (q, J = 7.99 Hz, 1 H) 1.83-1.95 (m, 2 H) 1.73-1.83 (m, 1 H) 1.56-1.63 (m, 2 H) 1.50 (d, J = 6.87 Hz, 6 H) 1.43-1.50 (m, 2 H) 1.36 (s, 9 H) 1.34 (dd, J = 7.02, 1.53 Hz, 6 H) 1.19-1.31 (m, 4 H). LC-MS: purity 100% (UV), t_{R} 5.20 min m/z [M + H]⁺ 871.75 (MET/CR/1416). |
| 1204 | 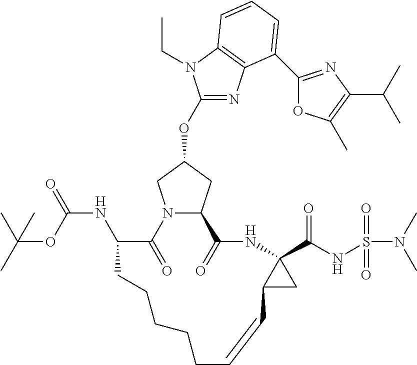 | 14.2 mg (14%) as an off-white solid after preparative HPLC. ¹H NMR (500 MHz, CDCl₃) δ ppm 9.97 (br. s., 1 H) 7.79-7.90 (m, 1 H) 7.16-7.25 (m, 2 H) 6.68-6.87 (m, 1 H) 6.09 (br. s., 1 H) 5.67-5.82 (m, 1 H) 4.92-5.11 (m, 2 H) 4.61 (t, J = 7.93 Hz, 1 H) 4.51 (d, J = 11.90 Hz, 1 H) 4.27 (t, J = 7.02 Hz, 1 H) 3.91-4.13 (m, 3 H) 2.92-3.06 (m, 1H) 2.88 (s, 6 H) 2.65-2.83 (m, 2 H) 2.49-2.62 (m, 1 H) 2.43 (s, 3 H) 2.18-2.29 (m, 1H) 1.86-1.95 (m, 2 H) 1.70-1.80 (m, 2 H) 1.54-1.67 (m, 1 H) 1.43-1.54 (m, 3 H) 1.37-1.43 (m, 2 H) 1.34 (br. s., 9 H) 1.31-1.33 (m, 6 H) 1.17-1.31 (m, 4 H). LC-MS: purity 100% (UV), t_{R} 5.14 min m/z [M + H]⁺ 839.30 (MET/CR/1416). |
| 1205 | 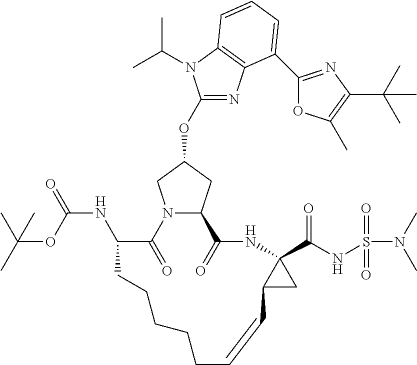 | 44 mg (34%) as an off-white solid after preparative HPLC. ¹H NMR (500 MHz, CDCl₃) δ ppm 9.73-10.10 (m, 1 H) 7.64-8.10 (m, 1 H) 7.29-7.37 (m, 1 H) 7.17-7.24 (m, 1 H) 6.63-6.88 (m, 1 H) 6.03 (br. s., 1 H) 5.69-5.83 (m, 1 H) 4.96-5.11 (m, 2 H) 4.45-4.66 (m, 3 H) 4.24-4.35 (m, 1 H) 3.95-4.14 (m, 1 H) 2.88 (s, 6 H) 2.80-2.87 (m, 1 H) 2.67-2.78 (m, 1 H) 2.54 (s, 3 H) 2.48-2.63 (m, 1 H) 2.22-2.31 (m, 1 H) 2.06-2.22 (m, 3 H) 1.72-1.96 (m, 3 H) 1.57-1.66 (m, 1 H) 1.52 (d, J = 6.71 Hz, 6 H) 1.43 (s, 9 H) 1.36 (s, 9 H) 1.15-1.56 (m, 4 H). LC-MS: purity 100% (UV), t_{R} 4.57 min m/z [M + H]⁺ 867.41 (MET/CR/1426). |
| 1206 | 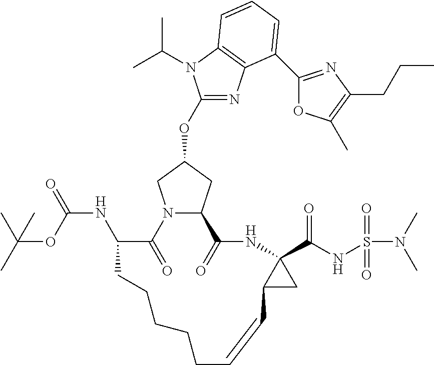 | 43 mg (31%) as an off-white solid after preparative HPLC. ¹H NMR (500 MHz, CDCl₃) δ ppm 9.83-10.28 (m, 1 H) 7.81 (d, J = 7.63 Hz, 1 H) 7.24-7.39 (m, 1 H) 7.10-7.23 (m, 1 H) 6.97 (s, 1 H) 6.09 (br. s., 1 H) 5.73 (q, J = 8.95 Hz, 1 H) 5.15 (d, J = 7.32 Hz, 1 H) 4.90-5.07 (m, 1 H) 4.53-4.65 (m, 2 H) 4.48 (d, J = 11.60 Hz, 1 H) 4.24-4.38 (m, 1 H) 3.95-4.23 (m, 1 H) 2.85 (s, 6 H) 2.72-2.80 (m, 1 H) 2.62-2.72 (m, 1 H) 2.53-2.61 (m, 1 H) 2.51 (t, J = 7.48 Hz, 2 H) 2.38 (s, 3 H) 2.21-2.32 (m, 1 H) 1.81-1.93 (m, 2 H) 1.75-1.81 (m, 1 H) 1.66-1.77 (m, 2 H) 1.53-1.63 (m, 1 H) 1.50 (d, J = 6.71 Hz, 6 H) 1.34 (s, 9 H) 1.14-1.47 (m, 7 H) 0.97 (t, J = 7.32 Hz, 3 H). LC-MS: purity 100% (UV), t_{R} 4.29 min m/z [M + H]⁺ 852.42 (MET/CR/1426). |
| 1207 | 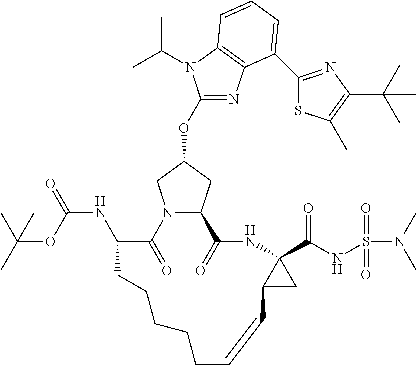 | 41 mg (40%) as an off-white solid after preparative HPLC. ¹H NMR (500 MHz, CDCl₃) δ ppm 9.89-10.42 (m, 1 H) 7.98-8.19 (m, 1 H) 7.22-7.26 (m, 1 H) 7.15-7.22 (m, 1 H) 6.73-7.05 (m, 1 H) 5.95 (br. s., 1 H) 5.74 (q, J = 8.85 Hz, 1 H) 5.16 (d, J = 7.63 Hz, 1 H) 5.03 (t, J = 9.16 Hz, 1 H) 4.55-4.65 (m, 2 H) 4.51 (d, J = 11.60 Hz, 1 H) 4.33 (d, J = 6.41 Hz, 1 H) 4.09-4.26 (m, 1 H) 2.86 (s, 6 H) 2.76-2.84 (m, 1 H) 2.66-2.75 (m, 1H) 2.60 (s, 3 H) 2.51-2.59 (m, 1 H) 2.18-2.33 (m, 1 H) 1.84-1.97 (m, 2 H) 1.72-1.83 (m, 1 H) 1.57-1.69 (m, 1 H) 1.53 (d, J = 6.71 Hz, 6 H) 1.49 (s, 9 H) 1.36 (s, 9 H) 1.12-1.57 (m, 7 H). LC-MS: purity 100% (UV), t_{R} 4.78 min m/z [M + H]⁺ 882.41 (MET/CR/1426). |
| 1208 | 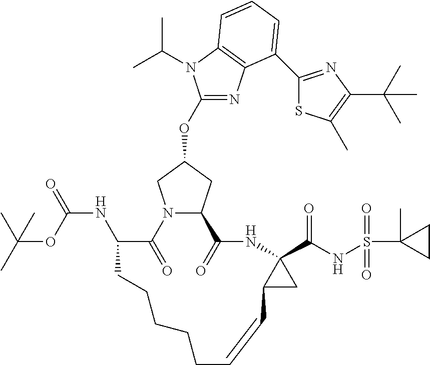 | 47 mg (46%) as an off-white solid after preparative HPLC. ¹H NMR (500 MHz, CDCl₃) δ ppm 10.22 (br. s., 1 H) 8.09 (d, J = 7.93 Hz, 1 H) 7.25-7.33 (m, 1 H) 7.17-7.25 (m, 1 H) 7.06 (br. s., 1 H) 5.94 (br. s., 1 H) 5.72 (q, J = 8.65 Hz, 1 H) 4.90-5.18 (m, 2 H) 4.57-4.68 (m, 2 H) 4.51 (d, J = 11.60 Hz, 1 H) 4.28-4.39 (m, 1 H) 4.21 (br. s., 1H) 2.80-2.92 (m, 1 H) 2.69-2.80 (m, 1 H) 2.61 (s, 3 H) 2.46-2.58 (m, 1 H) 2.22-2.35 (m, 1 H) 1.88-2.00 (m, 2 H) 1.72-1.87 (m, 2 H) 1.60-1.71 (m, 1 H) 1.54 (d, J = 7.02 Hz, 6 H) 1.51 (br. s., 6 H) 1.50 (br. s., 9 H) 1.39-1.58 (m, 4 H) 1.37 (s, 9 H) 1.14-1.26 (m, 1 H) 0.77-0.87 (m, 2 H). LC-MS: purity 99% (UV), t_{R} 4.86 min m/z [M + H]⁺ 894.42 (MET/CR/1426). |
| 1209 | 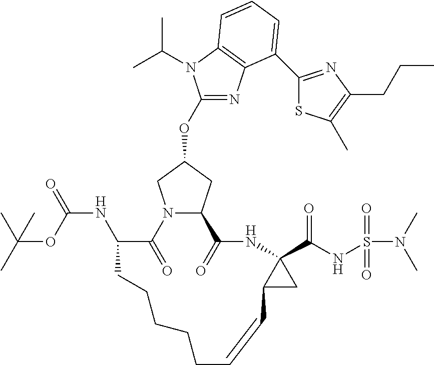 | 72 mg (54%) as a beige solid after preparative HPLC. ¹H NMR (500 MHz, CDCl₃) δ ppm 9.94-10.36 (m, 1 H) 8.09 (d, J = 6.71 Hz, 1 H) 7.22-7.29 (m, 1 H) 7.12-7.22 (m, 1 H) 6.98 (br. s.,1 H) 5.94 (br. s.,1 H) 5.73 (q, J = 8.75 Hz, 1 H) 5.21 (d, J = 7.63 Hz, 1 H) 5.01 (t, J = 9.46 Hz, 1 H) 4.54-4.67 (m, 2 H) 4.50 (d, J = 11.60 Hz, 1 H) 4.26-4.40 (m, 1 H) 4.07-4.25 (m, 1 H) 2.84 (s, 6 H) 2.77-2.90 (m, 1 H) 2.74 (t, J = 7.48 Hz, 2 H) 2.63-2.71 (m, 1 H) 2.51-2.62 (m, 1 H) 2.45 (s, 3 H) 2.18-2.33 (m, 1 H) 1.82-1.97 (m, 2 H) 1.70-1.82 (m, 4 H) 1.55-1.67 (m, 1 H) 1.52 (d, J = 6.71 Hz, 6 H) 1.39-1.50 (m, 5 H) 1.35 (s, 9 H) 1.23-1.32 (m, 1 H) 0.98 (t, J = 7.32 Hz, 3 H). LC-MS: purity 98% (UV), t_{R} 4.61 min m/z [M + H]⁺ 869.37 (MET/CR/1426). |
| 1210 | 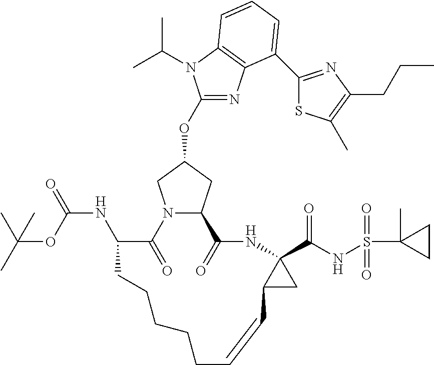 | 67 mg (50%) as a beige solid after preparative HPLC. ¹H NMR (500 MHz, CDCl₃) δ ppm 10.22 (br. s., 1 H) 8.09 (d, J = 6.41 Hz, 1 H) 7.24 (d, J = 7.93 Hz, 1 H) 7.14-7.22 (m, 1 H) 7.11 (br. s., 1 H) 5.96 (br. s., 1 H) 5.70 (q, J = 8.75 Hz, 1 H) 5.21 (d, J = 7.63 Hz, 1 H) 5.00 (t, J = 9.46 Hz, 1 H) 4.54-4.81 (m, 2 H) 4.28-4.54 (m, 2 H) 4.09-4.27 (m, 1 H) 2.77-2.87 (m, 1 H) 2.74 (t, J = 7.48 Hz, 2 H) 2.70 (d, J = 4.58 Hz, 1 H) 2.48-2.61 (m, 1 H) 2.45 (s, 3 H) 2.31 (d, J = 8.54 Hz, 1 H) 1.84-1.98 (m, 2 H) 1.70-1.84 (m, 4 H) 1.56-1.68 (m, 1 H) 1.52 (d, J = 6.71 Hz, 6 H) 1.48 (s, 3 H) 1.40-1.56 (m, 5 H) 1.37 (s, 9 H) 1.24-1.41 (m, 2 H) 1.12-1.23 (m, 3 H) 0.98 (t, J = 7.32 Hz, 3 H). LC-MS: purity 100% (UV), t_{R} 4.66 min m/z [M + H]⁺ 880.37 (MET/CR/1426). |
| 1211 | 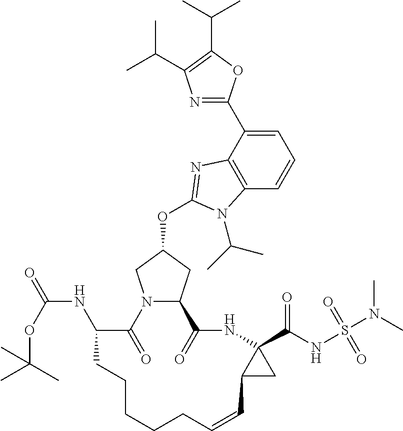 | 38 mg (31%) as an off-white solid after preparative HPLC. ¹H NMR (500 MHz, CDCl₃) δ ppm 9.67-10.29 (m, 1 H) 7.66-8.04 (m, 1 H) 7.30 (d, J = 7.48 Hz, 1 H) 7.14-7.24 (m, 1 H) 6.56-6.87 (m, 1 H) 5.90-6.09 (m, 1 H) 5.76 (q, J = 9.10 Hz, 1 H) 4.92-5.11 (m, 2 H) 4.48-4.75 (m, 3 H) 4.22-4.36 (m, 1 H) 3.93-4.15 (m, 1 H) 3.17 (spt, J = 7.02 Hz, 1 H) 2.95-3.10 (m, 1 H) 2.89-2.95 (m, 1 H) 2.88 (s, 6 H) 2.66-2.78 (m, 1 H) 2.49-2.62 (m, 1 H) 2.27 (q, J = 8.39 Hz, 1 H) 1.83-2.03 (m, 6 H) 1.72-1.83 (m, 1 H) 1.56-1.68 (m, 1 H) 1.52 (d, J = 6.41 Hz, 6 H) 1.44-1.49 (m, 2 H) 1.36-1.41 (m, 6 H) 1.36 (br. s., 9 H) 1.34 (d, J = 7.02 Hz, 6 H) 1.09-1.20 (m, 1 H). LC-MS: purity 100% (UV), t_{R} 4.66 min m/z [M + H]⁺ 881.41 (MET/CR/1426). |
| 1212 | 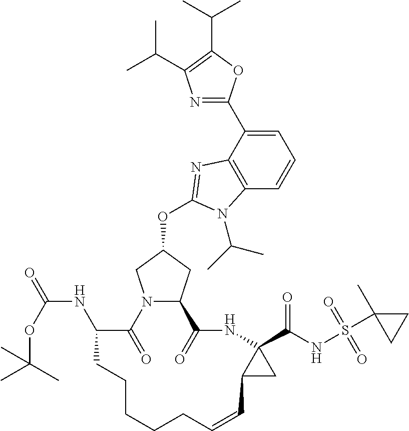 | 32 mg (26%) as an off-white solid after preparative HPLC. ¹H NMR (500 MHz, CDCl₃) δ ppm 9.90-10.31 (m, 1 H) 7.66-8.04 (m, 1 H) 7.28-7.39 (m, 1 H) 7.15-7.25 (m, 1 H) 6.58-6.86 (m, 1 H) 5.90-6.10 (m, 1 H) 5.74 (q, J = 8.80 Hz, 1 H) 4.94-5.10 (m, 2 H) 4.47-4.74 (m, 3 H) 4.25-4.35 (m, 1H) 3.93-4.16 (m, 1 H) 3.17 (spt, J = 7.02 Hz, 1 H) 2.96-3.10 (m, 1 H) 2.88-2.96 (m, 1 H) 2.67-2.79 (m, 1 H) 2.48-2.62 (m, 1 H) 2.30 (q, J = 8.70 Hz, 1 H) 1.87-1.98 (m, 2 H) 1.79-1.86 (m, 6H) 1.73-1.78 (m, 1 H) 1.57-1.68 (m, 1 H) 1.51-1.56 (m, 6 H) 1.50-1.51 (m, 1 H) 1.43-1.48 (m, 2 H) 1.37-1.42 (m, 6 H) 1.36 (br. s., 9 H) 1.34 (d, J = 6.87 Hz, 6 H) 1.25-1.31 (m, 2H) 1.10- 1.20 (m, 1 H) 0.78-0.87 (m, 2 H). LC-MS: purity 100% (UV), t_{R} 4.79 min m/z [M + H]⁺ 892.40 (MET/CR/1426). |
| 1213 | 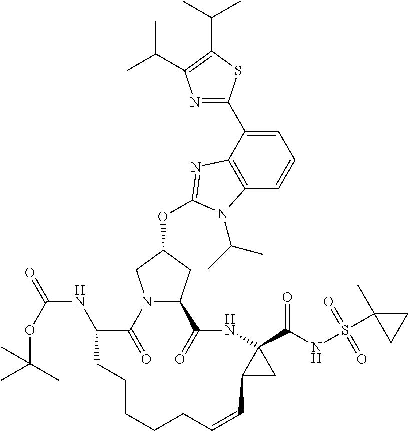 | 49 mg (36%) as an off-white solid after preparative HPLC. ¹H NMR (500 MHz, CDCl₃) δ ppm 9.86-10.48 (m, 1 H) 7.81-8.29 (m, 1 H) 7.22-7.27 (m, 1 H) 7.14-7.23 (m, 1 H) 6.62-6.87 (m, 1 H) 5.91-6.11 (m, 1 H) 5.74 (q, J = 8.80 Hz, 1 H) 4.94-5.11 (m, 2 H) 4.49-4.79 (m, 3 H) 4.26-4.41 (m, 1H) 4.00-4.16 (m, 1 H) 3.26-3.42 (m, 1 H) 3.05-3.24 (m, 1 H) 2.81-2.92 (m, 1 H) 2.67-2.81 (m, 1 H) 2.46-2.64 (m, 1 H) 2.26-2.35 (m, 1 H) 1.86-1.98 (m, 2 H) 1.71-1.87 (m, 3 H) 1.57-1.72 (m, 3 H) 1.53 (d, J = 6.56 Hz, 6 H) 1.50-1.52 (m, 2 H) 1.45-1.49 (m, 2 H) 1.42-1.45 (m, 1 H) 1.38-1.45 (m, 6 H) 1.38 (d, J = 3.05 Hz, 6 H) 1.36 (br. s., 9 H) 1.24-1.32 (m, 2 H) 1.11-1.22 (m, 1 H) 0.77-0.87 (m, 2 H). LC-MS: purity 100% (UV), t_{R} 5.10 min m/z [M + H]⁺ 908.39 (MET/CR/1426). |
| 1214 | 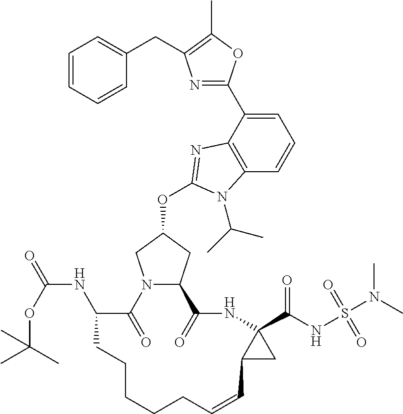 | 47 mg (29%) as an off-white solid after preparative HPLC. ¹H NMR (500 MHz, CDCl₃) δ ppm 9.92 (br. s.,1 H) 7.82 (d, J = 7.78 Hz, 1 H) 7.33 (d, J = 7.17 Hz, 2 H) 7.31 (d, J = 7.32 Hz, 2 H) 7.28-7.31 (m, 1 H) 7.22 (d, J = 7.17 Hz, 1 H) 7.18 (m, J = 7.93 Hz, 1 H) 6.68 (br. s., 1 H) 6.08 (br. s., 1 H) 5.76 (q, J = 9.16 Hz, 1 H) 4.98-5.07 (m, 2 H) 4.53-4.65 (m, 2 H) 4.50 (d, J = 11.75 Hz, 1 H) 4.25-4.32 (m, 1 H) 4.02 (dd, J = 11.60, 2.75 Hz, 1 H) 3.96 (s, 2 H) 2.88 (s, 6 H) 2.66-2.81 (m, 2 H) 2.51-2.63 (m, 1 H) 2.33 (s, 3 H) 2.25 (q, J = 8.44 Hz, 1 H) 1.83-1.95 (m, 2 H) 1.72-1.83 (m, 1 H) 1.55-1.62 (m, 1 H) 1.52 (d, J = 6.87 Hz, 6 H) 1.44-1.49 (m, 2 H) 1.38-1.44 (m, 2 H) 1.35 (s, 9 H) 1.23-1.32 (m, 2 H) 1.18 (br. s., 1 H). LC-MS: purity 100% (UV), t_{R} 4.67 min m/z [M + H]⁺ 901.95 (MET/CR/1426). |
| 1215 | 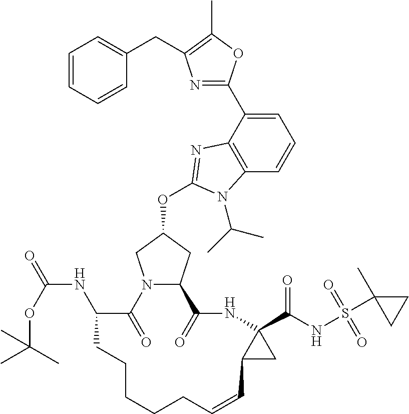 | 53 mg (32%) as an off-white solid after preparative HPLC. ¹H NMR (500 MHz, CDCl₃) δ ppm 9.92-10.14 (m, 1 H) 7.83 (d, J = 7.63 Hz, 1 H) 7.28-7.35 (m, 5 H) 7.22 (d, J = 7.02 Hz, 1 H) 7.19 (t, J = 7.78 Hz, 1 H) 6.73 (br. s., 1 H) 6.09 (br. s., 1 H) 5.74 (q, J = 8.80 Hz, 1 H) 4.98-5.07 (m, 2 H) 4.53-4.64 (m, 2 H) 4.50 (d, J = 11.75 Hz, 1 H) 4.26-4.33 (m, 1 H) 4.00-4.07 (m, 1 H) 3.96 (s, 2 H) 2.66-2.81 (m, 2 H) 2.50-2.61 (m, 1 H) 2.33 (s, 3 H) 2.25-2.32 (m, 1 H) 1.85-1.97 (m, 2 H) 1.73-1.85 (m, 3 H) 1.55-1.64 (m, 2 H) 1.52 (d, J = 7.02 Hz, 6 H) 1.49 (s, 3 H) 1.44-1.47 (m, 1 H) 1.38-1.44 (m, 2 H) 1.35 (s, 9 H) 1.27 (br. s., 2 H) 1.14-1.23 (m, 1 H) 0.78-0.86 (m, 2 H). LC-MS: purity 100% (UV), t_{R} 4.72 min m/z [M + H]⁺ 912.75 (MET/CR/1426). |
| 1216 | 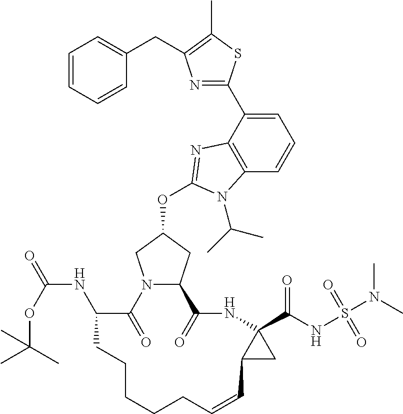 | 74 mg (55%) as an off-white solid after preparative HPLC. ¹H NMR (500 MHz, CDCl₃) δ ppm 10.00 (s, 1 H) 8.10 (br. s., 1 H) 7.24-7.32 (m, 5 H) 7.15-7.23 (m, 2 H) 6.65-6.72 (m, 1 H) 5.96 (br. s., 1 H) 5.76 (q, J = 9.16 Hz, 1 H) 4.99-5.07 (m, 2 H) 4.49-4.66 (m, 3 H) 4.28-4.35 (m, 1 H) 4.21 (br. s., 2 H) 4.10-4.17 (m, 1 H) 2.89 (s, 6 H) 2.80-2.86 (m, 1 H) 2.71-2.80 (m, 1 H) 2.53-2.63 (m, 1 H) 2.46 (s, 3 H) 2.24 (q, J = 8.34 Hz, 1 H) 1.84-1.95 (m, 2 H) 1.75-1.84 (m, 1 H) 1.57-1.66 (m, 1 H) 1.54 (d, J = 6.71 Hz, 6 H) 1.40-1.52 (m, 4 H) 1.36 (s, 9 H) 1.26-1.33 (m, 2 H) 1.11-1.22 (m, 1 H). LC-MS: purity 98% (UV), t_{R} 4.97 min m/z [M + H]⁺ 917.70 (MET/CR/1426). |
| 1217 | 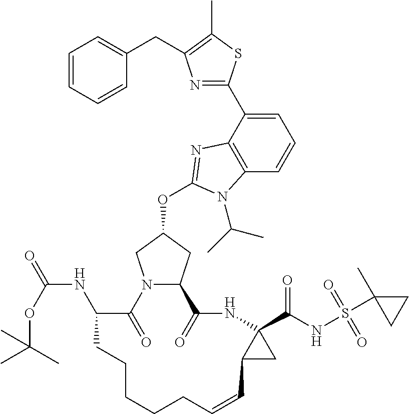 | 70 mg (51%) as an off-white solid after preparative HPLC. ¹H NMR (500 MHz, CDCl₃) δ ppm 10.13 (s, 1 H) 8.03-8.19 (m, 1 H) 7.25-7.34 (m, 5 H) 7.16-7.24 (m, 2 H) 6.75 (br. s., 1 H) 5.98 (br. s., 1 H) 5.75 (q, J = 8.85 Hz, 1 H) 4.98-5.11 (m, 2 H) 4.51-4.66 (m, 3 H) 4.29-4.38 (m, 1 H) 4.21 (br. s., 2 H) 4.12-4.19 (m, 1 H) 2.70-2.92 (m, 2 H) 2.52-2.64 (m, 1 H) 2.47 (s, 3 H) 2.30 (q, J = 8.70 Hz, 1 H) 1.88-1.99 (m, 2 H) 1.76-1.87 (m, 2 H) 1.58-1.67 (m, 2 H) 1.55 (d, J = 6.87 Hz, 6 H) 1.51 (s, 3 H) 1.40-1.50 (m, 4 H) 1.37 (s, 9 H) 1.24-1.34 (m, 2 H) 1.14-1.23 (m, 1 H) 0.80-0.87 (m, 2 H). LC-MS: purity 98% (UV), t_{R} 5.03 min m/z [M + H]⁺ 928.80 (MET/CR/1426). |

### 2.41 Synthesis of Compound 1218

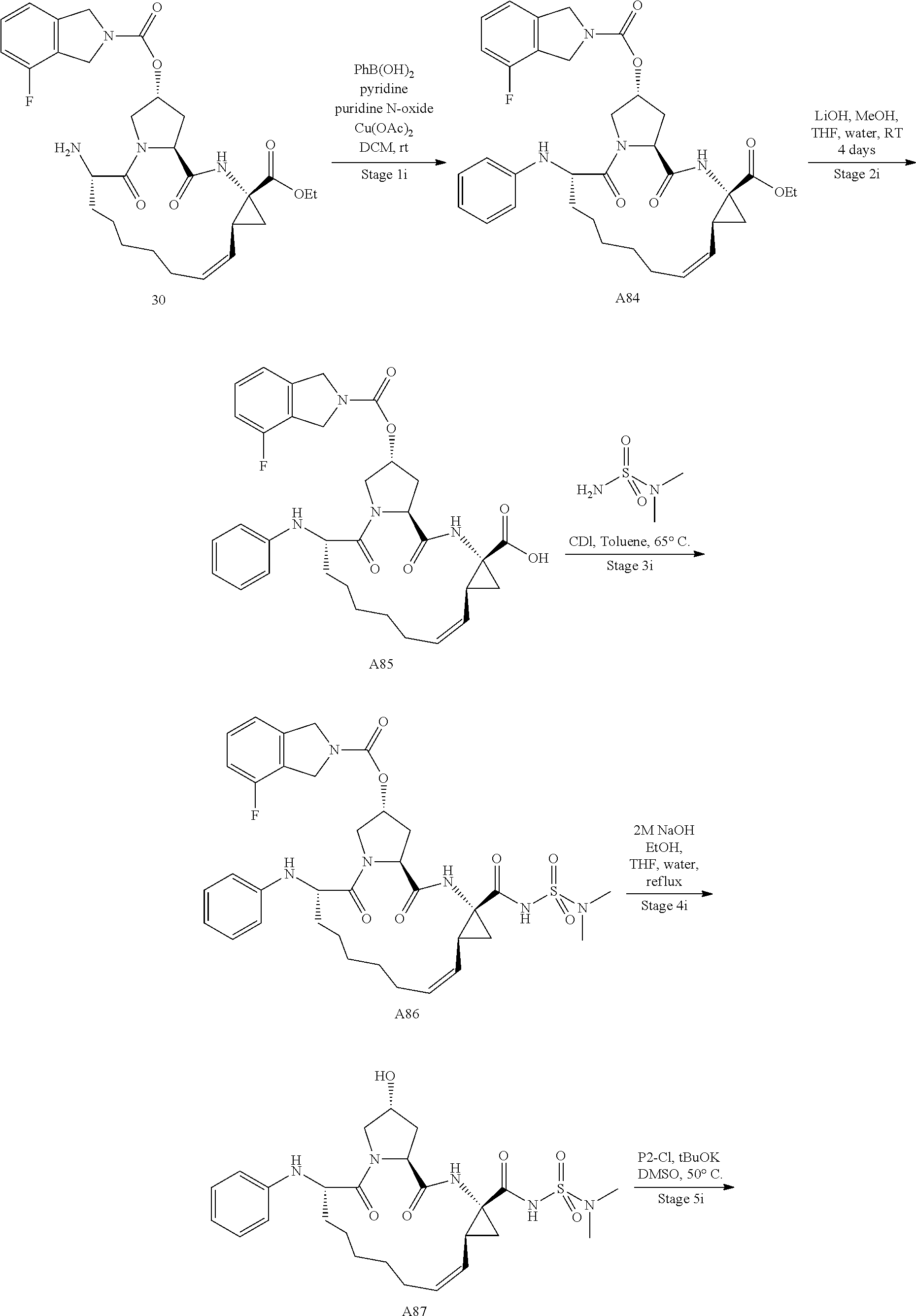

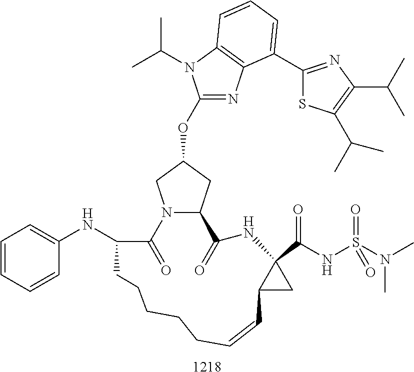

Stage 1i: The P4 amino macrocycle intermediate 30 (500 mg, 0.90 mmol, 1.0 eq.), pyridine-N-oxide (436 mg, 4.49 mmol, 5.0 eq.), pyridine (0.726 mL, 8.98 mmol, 10 eq.), phenyl boronic acid (328 mg, 2.69 mmol, 3.0 eq.), copper (II) acetate (326 mg, 1.80 mmol, 2.0 eq.), 4 Å molecular sieves, and dichloromethane (10 mL) were charged into a 25 mL flask. The reaction mixture was stirred at ambient temperature under an air atmosphere for 15 hours. The sieves were removed by filtration and the reaction mixture acidified to pH 2-3 by addition of 1 M hydrochloric acid. The two phases were separated and the aqueous phase further extracted with dichloromethane (10 mL). The combined organic extracts were dried over sodium sulfate, filtered and the solvent removed in vacuo. The residue was purified by flash column chromatography (methanol/dichloromethane gradient) to give 310 mg (54% yield) of compound A84 as a yellow oil. ¹H NMR (500 MHz, CDCl₃) δ ppm 7.77-7.94 (m, 1H) 7.22-7.34 (m, 1H) 7.02-7.18 (m, 3H) 6.90-7.00 (m, 1H) 6.47-6.71 (m, 3H) 5.46-5.61 (m, 1H) 5.37 (br. s., 1H) 5.27 (t, J=9.61 Hz, 1H) 4.78-4.87 (m, 1H) 4.75 (d, J=6.71 Hz, 2H) 4.45-4.67 (m, 2H) 4.39 (t, J=7.02 Hz, 1H) 4.05-4.22 (m, 2H) 3.93-4.03 (m, 1H) 3.82-3.93 (m, 1H) 2.70-2.88 (m, 1H) 2.11-2.33 (m, 4H) 1.91-2.04 (m, 1 H) 1.84 (dt, J=8.16, 5.53 Hz, 1H) 1.64-1.79 (m, 1H) 1.56 (dd, J=9.46, 5.49 Hz, 1H) 1.12-1.51 (m, 10H). LC-MS: purity 96% (UV), t_{R} 2.39 min m/z [M+H]⁺ 633.75 (MET/CR/1278).

Stage 2i: Compound A84 (310 mg, 0.49 mmol, 1.0 eq.), methanol (7 mL), water (7 mL) and tetrahydrofuran (17 mL) were charged into a 50 mL round bottom flask and the reaction mixture cooled on top of an ice bath. Lithium hydroxide monohydrate (41 mg, 0.98 mmol, 2.0 eq.) was added portion wise and stirring of the cold solution was continued for 4 hours. The ice batch was removed and stirring continued at ambient temperature for 15 hours. As the reaction was not complete additional lithium hydroxide monohydrate (20 mg, 0.49 mmol, 1.0 eq.) was added and stirring continued at ambient temperature for another 24 hours by which time ˜80% conversion was reached. Lithium hydroxide monohydrate (20 mg, 0.49 mmol, 1.0 eq.) was added and stirring continued at ambient temperature for a further 72 hours by which time the reaction was complete. The reaction mixture was neutralised with 1 M aqueous acetic acid and extracted with dichloromethane (3×7 mL). The combined organic extracts were dried over magnesium sulfate, filtered and the solvent removed in vacuo to give 278 mg (93% yield) of compound A85 as a yellow foamy solid. ¹H NMR (500 MHz, CDCl₃) δ ppm 7.20-7.32 (m, 2H) 7.03-7.12 (m, 2H) 6.94-7.01 (m, 1H) 6.61 (d, J=7.63 Hz, 1 H) 6.49-6.59 (m, 1H) 5.54-5.68 (m, 1H) 5.38-5.46 (m, 1H) 5.19-5.26 (m, 1H) 4.69-4.84 (m, 3H) 4.55-4.64 (m, 1H) 4.45-4.54 (m, 1H) 4.36 (t, J=7.02 Hz, 1H) 3.89-4.01 (m, 2H) 2.66-2.80 (m, 1H) 2.11-2.32 (m, 4H) 1.88-2.02 (m, 2H) 1.79-1.88 (m, 1H) 1.66-1.79 (m, 1H) 1.53-1.66 (m, 1H) 1.16-1.53 (m, 9H). LC-MS: purity 96% (UV), t_{R} 2.17 min m/z [M+H]⁺ 605.55 (MET/CR/1278).

Stage 3i: Compound A85 (278 mg, 0.41 mmol, 1.0 eq.) and dry toluene (5 mL) were charged into a 50 mL round bottom flask. 1,1′-Carbonyldiimidazole (82 mg, 0.50 mmol, 1.2 eq.) was added and the reaction mixture heated at 65° C. for 2 hours. N,N-Dimethylsulfamide (63 mg, 0.50 mmol, 1.2 eq.) and 1,8-diazabicyclo[5.4.0]undec-7-ene (93 mg, 0.60 mmol, 1.5 eq.) were added and stirring continued at 65° C. for 1.5 hours and then at ambient temperature for 15 hours. The solvent was removed in vacuo. Water (5 mL) was added and the pH adjusted to 1 with 1 M hydrochloric acid. The aqueous phase was extracted with dichloromethane (2×5 mL) and the combined organic extracts dried over sodium sulfate, filtered and the solvent removed in vacuo. The residue was purified by flash column chromatography (methanol/dichloromethane gradient) to give 163 mg (55% yield) of compound A86 as a yellow oil. ¹H NMR (250 MHz, CDCl₃) δ ppm 9.87-10.21 (m, 1H) 7.40 (br. s., 1H) 7.22-7.38 (m, 1H) 6.84-7.18 (m, 4H) 6.33-6.70 (m, 3H) 5.76 (q, J=8.93 Hz, 1H) 5.62-5.85 (m, 1H) 5.48 (br. s., 1H) 5.02 (dd, J=10.36, 8.53 Hz, 1H) 4.65-4.88 (m, 2H) 4.35-4.66 (m, 3H) 4.12-4.29 (m, 1H) 3.89-4.09 (m, 2H) 2.87 (s, 6H) 2.04-2.68 (m, 6H) 1.64-2.03 (m, 3H) 1.06-1.63 (m, 6H). LC-MS: purity 91% (UV), t_{R} 2.40 min m/z [M+H]⁺ 711.55 (MET/CR/1278).

Stage 4i: Compound A86 (163 mg, 0.23 mmol, 1.0 eq.), 2 M aqueous sodium hydroxide (1.2 mL, 2.4 mmol, 10 eq.) and ethanol (4 mL) were charged into a 10 mL round bottom flask. The reaction mixture was heated under reflux for 2 hours then stirring was continued at ambient temperature for 60 hours. Ethanol was removed in vacuo. The pH of the remaining aqueous solution was adjusted to 4 with 0.2 M hydrochloric acid and the mixture extracted with dichloromethane (2×20 mL). The pH of the aqueous phase was adjusted to 1 with 0.2 M hydrochloric acid and the mixture extracted with ethyl acetate (20 mL). The dichloromethane and ethyl acetate extracts were combined, dried over magnesium sulfate, filtered and the solvent removed in vacuo to give 125 mg (100% yield) of compound A87 as a beige solid which was used in the next step without further purification. LC-MS: purity 99% (ELS), t_{R} 2.05 min m/z [M+H]⁺ 548.55 (MET/CR/1278).

Stage 5i: Compound A87 (125 mg, 0.23 mmol, 1.0 eq.), 1-isopropyl-2-Chloro-4-(4,5-diisopropyl-thiazol-2-yl)-benzimidazole (109 mg, 0.30 mmol, 1.3 eq) and anhydrous dimethylsulfoxide (4 mL) were charged into a 12 mL vial. Potassium tert-butoxide (135 mg, 1.20 mmol, 5.2 eq.) was added as a single portion and the reaction mixture stirred at ambient temperature for 15 hours. The reaction mixture was diluted with water (16 mL), 1 M hydrochloric acid (2 mL) and extracted with ethyl acetate (2×25 mL). The combined organic extracts were dried over magnesium sulfate, filtered and the solvent removed in vacuo. The residue was purified by preparative HPLC to give 52 mg (25% yield) of compound 1218 as a beige solid. ¹H NMR (500 MHz, CDCl₃) δ ppm 9.88-10.10 (m, 1H) 7.99-8.36 (m, 1H) 7.17-7.36 (m, 1H) 6.65-6.92 (m, 3H) 6.50-6.58 (m, 1H) 6.40-6.49 (m, 2H) 5.82-5.91 (m, 1H) 5.70-5.83 (m, 1H) 4.90-5.11 (m, 2H) 4.57-4.76 (m, 2H) 4.43-4.57 (m, 1 H) 4.21-4.31 (m, 1H) 4.07-4.21 (m, 1H) 3.30-3.48 (m, 1H) 2.89 (s, 6H) 2.77-2.86 (m, 1H) 2.65-2.76 (m, 1H) 2.44-2.63 (m, 1H) 2.10-2.25 (m, 1H) 1.88-1.99 (m, 3H) 1.76-1.88 (m, 2H) 1.56-1.63 (m, 1H) 1.49-1.56 (m, 4H) 1.45 (d, J=6.71 Hz, 6H) 1.39-1.43 (m, 9H) 1.36 (d, J=6.87 Hz, 3H) 1.27-1.35 (m, 3H). LC-MS: purity 95% (UV), t_{R} 5.06 min m/z [M+H]⁺ 873.33 (MET/CR/1426). 2.42 Synthesis of Compound 1219

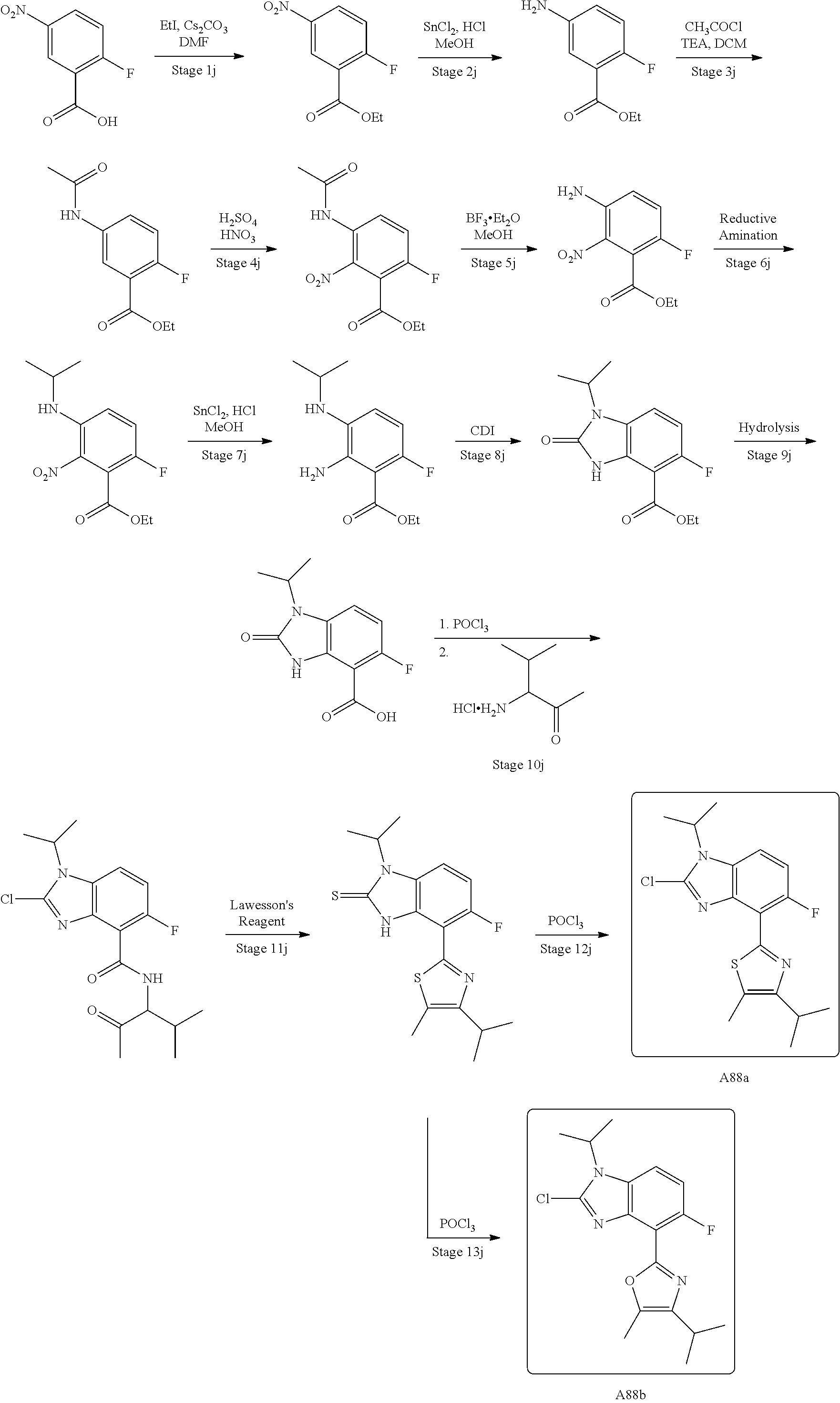

Stage 1j—Ethyl 2-Fluoro-5-nitrobenzoate: 2-Fluoro-5-nitro-benzoic acid (2.01 g, 10.8 mmol, 1.0 eq.), caesium carbonate (7.66 g, 21.71 mmol, 2.0 eq.) and N,N-dimethylformamide (20 mL) were charged into a 50 mL flask. Ethyl iodide (1.04 g, 13.03 mmol, 1.2 eq.) was added dropwise and the reaction mixture heated at 70° C. for 4 hours. The reaction mixture was poured into water (80 mL) and the solution extracted with ethyl acetate (3×20 mL). The organic extracts were combined, washed with water (5×20 mL) and brine (20 mL), dried over magnesium sulfate, filtered and the solvent removed in vacuo. The residue was purified by flash column chromatography (ethyl acetate/heptanes gradient) to give 1.9 g (82% yield) of Ethyl 2-Fluoro-5-nitrobenzoate as a colourless oil. ¹H NMR (250 MHz, CDCl₃) δ ppm 8.79 (dd, J=6.24, 2.89 Hz, 1H) 8.36 (ddd, J=9.06, 3.96, 2.97 Hz, 1H) 7.15-7.35 (m, 1H) 4.40 (q, J=7.16 Hz, 2H) 1.38 (t, J=7.16 Hz, 3H). LC-MS: purity 100% (UV), t_{R} 1.96 min no ionisation (MET/CR/1278).

Stage 2j—Ethyl 2-fluoro-5-aminobenzoate: Ethyl 2-Fluoro-5-nitrobenzoate (1.9 g, 9.05 mmol, 1.0 eq.) was dissolved in methanol (40 mL). Tin chloride dihydrate (10.2 g, 45.26 mmol, 5.0 eq.) was added portionwise and the reaction mixture heated under reflux for 2 hours then stirred at ambient temperature for 15 hours. The reaction mixture was cooled to 0° C. and quenched with concentrated aqueous ammonia (20 mL), leading to the formation of a white sticky solid. Celite® (1 g) was added to the reaction flask and the slurry was stirred for a further 10 minutes. The solid was removed by filtration and the cake extracted with dichloromethane (100 mL). The filtrate and the organic extract were combined and left to separate. The aqueous phase was discarded and the organic phase washed with water (50 mL) and brine (50 mL), dried over magnesium sulfate, filtered and the solvent removed in vacuo to give 1.69 g (100% yield) of the title compound as a yellow oil which was used in the next step without further purification. ¹H NMR (500 MHz, CDCl₃) δ ppm 7.20 (dd, J=5.72, 2.98 Hz, 1 H) 6.90-6.96 (m, 1H) 6.80 (dt, J=8.58, 3.41 Hz, 1H) 4.38 (q, J=7.17 Hz, 2H) 3.45-3.94 (m, 2H) 1.39 (t, J=7.10 Hz, 3H). LC-MS: purity 100% (UV), t_{R} 1.32 min m/z [M+H]⁺183.95 (MET/CR/1278).

Stage 3j—Ethyl 2-fluoro-5-acetylaminobenzoate: Ethyl 2-fluoro-5-aminobenzoate (1.69 g, 9.22 mmol, 1.0 eq.) and dichloromethane (35 mL) were charged into a 100 mL round bottom flask. Triethylamine (1.93 g, 13.83 mmol, 1.5 eq.) was added as a single portion and the reaction mixture cooled to 0° C. Acetyl chloride (1.31 g, 18.45 mmol, 2.0 eq.) was added dropwise and the reaction mixture stirred at 0° C. for a further 1 hour. The reaction mixture was washed with water (2×20 mL), saturated aqueous sodium hydrogen carbonate (20 mL) and brine (20 mL), dried over magnesium sulfate, filtered and the solvent removed in vacuo to give 1.66 g (80% yield) of the title compound as a yellow solid which was used in the next step without further purification. ¹H NMR (500 MHz, CDCl₃) δ ppm 7.81-7.93 (m, 2H) 7.29-7.42 (m, 1H) 7.06-7.16 (m, 1H) 4.35-4.44 (m, 2H) 2.16-2.23 (m, 3H) 1.38-1.43 (m, 3H). LC-MS: purity 92% (UV), t_{R} 1.62 min m/z [M+H]⁺225.90 (MET/CR/1278).

Stage 4j—Ethyl 2-nitro-3-acetylamino-6-fluoro-benzoate: Sulfuric acid (13 mL) was charged into a 50 mL flask and cooled to 0° C. Ethyl 2-fluoro-5-acetylaminobenzoate (1.65 g, 7.32 mmol, 1.0 eq.) was added portionwise to give an orange solution. Concentrated nitric acid (13 mL) was added dropwise over 10 minutes to the cold reaction mixture. Stirring was continued at 0° C. for another 30 minutes. LCMS analysis showed the reaction to be complete, but 2 isomers could be detected. Reaction mixture was carefully poured into crushed ice (200 g) and the slurry stirred with a glass rod leading to the precipitation of a sticky yellow gum. The aqueous mixture was extracted with ethyl acetate (3×100 mL). The organic extracts were combined, washed with water (100 mL), saturated aqueous sodium hydrogen carbonate (100 mL) and brine (100 mL), dried over magnesium sulfate, filtered and the solvent removed in vacuo to give a red oily residue which was purified by flash column chromatography (ethyl acetate/heptanes gradient) to give 825 mg (42% yield) of the title compound as a pale yellow solid. ¹H NMR (500 MHz, CDCl₃) δ ppm 9.25 (br. s., 1H) 8.61 (dd, J=9.38, 4.96 Hz, 1H) 7.36-7.46 (m, 1H) 4.44 (q, J=7.17 Hz, 2H) 2.27 (s, 3H) 1.39 (t, J=7.17 Hz, 3H). LC-MS: purity 100% (UV), t_{R} 1.69 min m/z [M+H]⁺ 270.95 (MET/CR/1278).

Stage 5j—Ethyl 2-nitro-3-amino-6-fluoro-benzoate: Ethyl 2-nitro-3-acetylamino-6-fluoro-benzoate (825 mg, 3.07 mmol, 1.0 eq.) and methanol were charged into a 50 mL round bottom flask. Boron trifluoride etherate (1.7 mL, 13.78 mmol, 4.5 eq.) was added dropwise at ambient temperature and the reaction mixture heated under reflux for 2 hours. The reaction mixture was neutralised with solid sodium hydrogen carbonate (3.5 g) and the solvent removed in vacuo. The residue was partitioned between water (45 mL) and ethyl acetate (30 mL). The organic phase was further washed with water (45 mL) and brine (50 mL), dried over magnesium sulfate and the solvent removed in vacuo to give 702 mg (100% yield) of the title compound as a yellow-orange solid which was used crude in the next step. ¹H NMR (500 MHz, CDCl₃) δ ppm 7.22 (dd, J=8.85, 7.93 Hz, 1H) 6.86 (dd, J=9.31, 4.73 Hz, 1H) 5.96 (br. s., 2H) 4.45 (q, J=7.07 Hz, 2H) 1.39 (t, J=7.17 Hz, 3H). LC-MS: purity 99% (UV), t_{R} 1.78 min m/z [M−H]⁻ 226.95 (MET/CR/1278).

Stage 6j—Ethyl 2-nitro-3-isopropylamino-6-fluoro-benzoate: Ethyl 2-nitro-3-amino-6-fluoro-benzoate (702 mg, 3.07 mmol, 1.0 eq.), dichloromethane (4 mL) and acetic acid (2 mL) were charged into 10 ml round bottom flask. Acetone (360 mg, 4.92 mmol, 1.6 eq.) was added and the reaction mixture stirred at ambient temperature for 5 minutes. The reaction mixture was cooled to 0° C. and borane dimethylsulfide complex (350 mg, 3.69 mmol, 1.2 eq.) was added dropwise. The reaction mixture was then stirred at ambient temperature for a further 15 hours. The reaction mixture was cooled down to 0° C. and quenched with saturated aqueous ammonium chloride (1.5 mL). The organic layer was washed with brine (20 mL), dried over magnesium sulfate, filtered and the solvent removed in vacuo to give 799 mg (96% yield) of the title compound as a dark yellow solid which was used crude in the next step. ¹H NMR (500 MHz, CDCl₃) δ ppm 7.86 (d, J=6.41 Hz, 1H) 7.22-7.32 (m, 1H) 6.91 (dd, J=9.69, 4.65 Hz, 1H) 4.44 (q, J=7.17 Hz, 2H) 3.75-3.87 (m, 1H) 1.39 (t, J=7.17 Hz, 3H) 1.32 (d, J=6.41 Hz, 6H). LC-MS: purity 100% (UV), t_{R} 2.17 min m/z [M+H]⁺ 271.00 (MET/CR/1278).

Stage 7j—Ethyl 2-amino-3-isopropylamino-6-fluoro-benzoate: Ethyl 2-nitro-3-isopropylamino-6-fluoro-benzoate (690 mg, 2.55 mmol, 1.0 eq.) and methanol (7 mL) were charged into a 25 mL round bottom flask. Tin chloride dihydrate (225 g, 2.88 mmol, 5.0 eq.) was added portionwise and the reaction mixture heated under reflux for 2 hours. The reaction mixture was cooled to 0° C. and quenched with concentrated aqueous ammonia (2 mL). The obtained slurry was filtered over a pad of Celite®. The solid was washed with dichloromethane (15 mL). The filtrate and the organic washing were combined and left to separate. The aqueous phase was discarded and the organic phase washed with water (15 mL) and brine (15 mL), dried over magnesium sulfate, filtered and the solvent removed in vacuo to give 540 mg (88% yield) of the title compound as a yellow syrup which was used in the next step without further purification. ¹H NMR (500 MHz, CDCl₃) δ ppm 6.76 (dd, J=8.24, 5.04 Hz, 1H) 6.37 (dd, J=11.29, 8.55 Hz, 1H) 5.71 (br. s., 2H) 4.38 (q, J=7.17 Hz, 2H) 3.46 (spt, J=6.21 Hz, 1H) 1.63 (br. s., 1H) 1.40 (t, J=7.17 Hz, 3H) 1.19 (d, J=6.26 Hz, 6H). LC-MS: purity 98% (UV), t_{R} 1.79 min m/z [M+H]⁺ 241.05 (MET/CR/1278).

Stage 8j—1-isopropyl-2-oxo-4-ethoxycarbonyl-5-fluoro-benzimidazole: Ethyl 2-amino-3-isopropylamino-6-fluoro-benzoate (540 mg, 2.25 mmol, 1.0 eq.) and tetrahydrofuran (3 mL) were charged into a 10 mL vial. 1,1′-carbodimidazole (546 mg, 3.37 mmol, 1.5 eq.) was added as a single portion and the reaction mixture heated under reflux for 15 hours. The reaction mixture was left to cool down to ambient temperature and was diluted with 2 M hydrochloric acid (4 mL). The solution was extracted with ethyl acetate (10×3 mL). The organic extracts were combined, washed with water (10 mL) and brine (10 mL), dried over magnesium sulfate, filtered and the solvent removed in vacuo to give 600 mg (100% yield) of the title compound as a yellow solid which was used in the next step without further purification. ¹H NMR (500 MHz, CDCl₃) d ppm 9.14 (br. s., 1H) 7.16 (dd, J=8.62, 3.74 Hz, 1H) 6.82 (dd, J=11.75, 8.70 Hz, 1H) 4.71 (spt, J=7.04 Hz, 1H) 4.45 (q, J=7.17 Hz, 2H) 1.53 (d, J=7.02 Hz, 6H) 1.43 (t, J=7.17 Hz, 3H). LC-MS: purity 97% (UV), t_{R} 1.88 min m/z [M+H]⁺ 267.00 (MET/CR/1278).

Stage 9j—1-isopropyl-2-oxo-4-carboxyl-5-fluoro-benzimidazole lithium salt: 1-isopropyl-2-oxo-4-ethoxycarbonyl-5-fluoro-benzimidazole (600 mg, 2.25 mmol, 1.0 eq.), methanol (0.3 mL) and tetrahydrofuran (0.6 mL) were charged into a 7 mL vial. Lithium hydroxide monohydrate (472 mg, 11.3 mmol, 5 eq.) was dissolved in water (0.3 mL) and the solution added as a single portion to the reaction mixture. The reaction mixture was then heated at 70° C. for 2 hours. The solvent was removed in vacuo and the residue azeotroped twice with toluene (5 mL) to give 540 mg (100% yield) of the title compound as an off-white solid. LC-MS: purity 97% (UV), t_{R} 1.51 min m/z [M+H]⁺ 238.95 (MET/CR/1278).

Stage 10j—1-isopropyl-2-Chloro-4-[(4-methyl-pent-2-on-3-yl)-aminocarbonyl]-5-fluoro-benzimidazole: 1-isopropyl-2-oxo-4-carboxyl-5-fluoro-benzimidazole lithium salt (65 mg, 0.27 mmol, 1.0 eq.) and phosphorous oxychloride (1 mL) were charged into 7 mL vial. The reaction mixture was heated at 110° C. for 15 hours then the solvent removed in vacuo. Dry dioxane (3 mL) was added to the residue followed by diisopropylethyl amine (0.149 mL, 0.85 mmol, 3 eq.) and 1-amino-4-methyl-pent-2-one hydrochloride (59 mg, 0.39 mmol, 1.5 eq.) and the reaction mixture stirred at ambient temperature for 15 hours. The reaction mixture was diluted with water (5 mL) and extracted with ethyl acetate (3×5 mL). The organic extracts were combined, washed with water (5 mL) and brine (5 mL) dried over magnesium sulfate, filtered and the solvent removed in vacuo to give 44 mg (47% yield) of the title compound as a sticky gum. ¹H NMR (500 MHz, CDCl₃) δ ppm 9.67 (d, J=7.48 Hz, 1H) 7.56 (dd, J=9.00, 3.66 Hz, 1H) 7.11 (m, J=11.75, 9.00 Hz, 1H) 4.94 (spt, J=6.99 Hz, 1H) 4.78 (dd, J=7.78, 4.43 Hz, 1H) 2.35-2.48 (m, 1 H) 2.28 (s, 3H) 1.56-1.76 (m, 6H) 0.97-1.15 (m, 6H). LC-MS: purity 92% (UV), t_{R} 2.11 min m/z [M+H]⁺ 354.45 (MET/CR/1278).

Stage 11j—1-isopropyl-2-thioxo-4-(4-isopropyl-5-methyl-thiazol-2-yl)-5-fluoro-benzimidazole: 1-isopropyl-2-Chloro-4-[(4-methyl-pent-2-on-3-yl)-aminocarbonyl]-5-fluoro-benzimidazole (41 mg, 0.122 mmol, 1.0 eq.) and Lawesson's reagent (59 mg, 0.146 mmol, 1.2 eq.) were charged in a microwave tube. Dioxane (0.4 mL) was added and the tube heated at in a focus microwave (180° C./100 W) for 15 minutes. The solvent was removed in vacuo and the residue purified by flash column chromatography (10% ethyl acetate in heptanes) to give 22 mg (50% yield) of the title compound as a beige solid. ¹H NMR (500 MHz, CDCl₃) δ ppm 11.49 (br. s., 1H) 7.20 (dd, J=8.77, 3.89 Hz, 1H) 6.93 (dd, J=11.52, 8.77 Hz, 1H) 5.55 (m, J=14.15, 7.04, 7.04 Hz, 1H) 3.10 (spt, J=6.84 Hz, 1H) 2.39 (s, 3H) 1.52 (d, J=7.17 Hz, 6H) 1.26 (d, J=7.02 Hz, 6H). LC-MS: purity 90% (UV), t_{R} 2.43 min m/z [M+H]⁺ 350.40 (MET/CR/1278).

Stage 12j—1-isopropyl-2-Chloro-4-(4-isopropyl-5-methyl-thiazol-2-yl)-5-fluoro-benzimidazole: 1-isopropyl-2-thioxo-4-(4-isopropyl-5-methyl-thiazol-2-yl)-5-fluoro-benzimidazole (23 mg, 0.065 mmol, 1.0 eq.) was dissolved into phosphorous oxychloride (0.5 mL) and the reaction mixture heated at 110° C. for 15 hours. The solvent was removed in vacuo and the residue azeotroped with heptane. The residue was partitioned between dichloromethane (2 mL) and water (1 mL). Saturated aqueous sodium hydrogen carbonate was added (˜1 mL) until neutral pH was reached. Organic layer was separated, washed with water (1 mL). The aqueous layer was back extracted with dichloromethane (2×1 mL). The organic layers were combined, dried over magnesium sulfate, filtered and the solvent removed in vacuo to give 15 mg (65% yield) of the title compound A88a as a syrup which was used in the next step without further purification. ¹H NMR (500 MHz, CDCl₃) δ ppm 7.66 (d, J=5.19 Hz, 1H) 7.25 (d, J=9.61 Hz, 1H) 4.98 (spt, J=6.84 Hz, 1H) 3.28-3.45 (m, 1H) 2.57 (s, 3 H) 1.68 (m, J=7.02 Hz, 6H) 1.45 (m, J=6.87 Hz, 6H). LC-MS: purity 92% (UV), t_{R} 2.13 min m/z [M+H]⁺ 353.00 (MET/CR/1981).

Stage 13j—1-isopropyl-2-Chloro-4-(4-isopropyl-5-methyl-oxazol-2-yl)-5-fluoro-benzimidazole: 1-isopropyl-2-Chloro-4-[(4-methyl-pent-2-on-3-yl)-aminocarbonyl]-5-fluoro-benzimidazole (19 mg, 0.054 mmol, 1 eq.) was dissolved into phosphorous oxychloride (1 mL) and the reaction mixture heated at 110° C. for 24 hours. The solvent was removed in vacuo and the residue azeotroped with heptane to give 35 mg (>100% yield) of the title compound A88b as a light brown oil which was used in the next step without further purification. ¹H NMR (500 MHz, CDCl₃) δ ppm 8.24 (dd, J=9.08, 4.04 Hz, 1H) 7.47 (dd, J=11.22, 9.23 Hz, 1H) 5.16 (spt, J=6.84 Hz, 1H) 2.58-2.67 (m, 3H) 1.77-1.83 (m, 1H) 1.69-1.76 (m, 6H) 1.41-1.47 (m, 6H). LC-MS: purity 86% (UV), t_{R} 2.29 min m/z [M+H]⁺336.40 (MET/CR/1278).

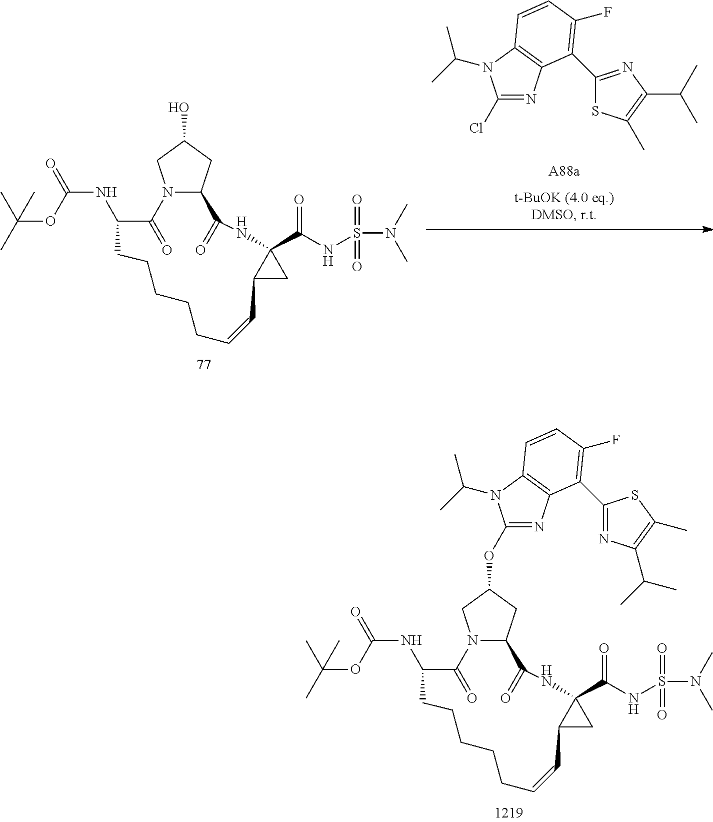

Compounds 1219 and 1220 were prepared using the same method for preparing compound 1201 described in section 2.39 above, using precursor compounds A88a and A88b, respectively. Yielded 13 mg (32%) of compound 1219 as a white solid after flash column chromatography. ¹H NMR (500 MHz, CDCl₃) δ ppm 10.00 (br. s., 1H) 7.12-7.21 (m, 1H) 6.98 (dd, J=11.22, 8.93 Hz, 1H) 6.77 (br. s., 1H) 5.90 (br. s., 1H) 5.75 (q, J=9.05 Hz, 1H) 4.96-5.14 (m, 2H) 4.50-4.62 (m, 2H) 4.26-4.36 (m, 1H) 4.03-4.24 (m, 1H) 3.19 (spt, J=6.71 Hz, 1H) 2.88 (s, 6H) 2.79-2.86 (m, 1H) 2.66-2.76 (m, 1H) 2.52-2.62 (m, 1H) 2.48 (s, 3H) 2.25 (q, J=8.80 Hz, 1H) 1.86-1.95 (m, 2H) 1.73-1.84 (m, 2H) 1.58-1.69 (m, 1H) 1.52 (d, J=6.87 Hz, 6H) 1.46-1.50 (m, 2H) 1.37-1.43 (m, 6H) 1.36 (br. s., 9H) 1.17-1.33 (m, 5H). LC-MS: purity 100% (UV), t_{R} 5.13 min m/z [M+H]⁺887.65 (MET/CR/1416).

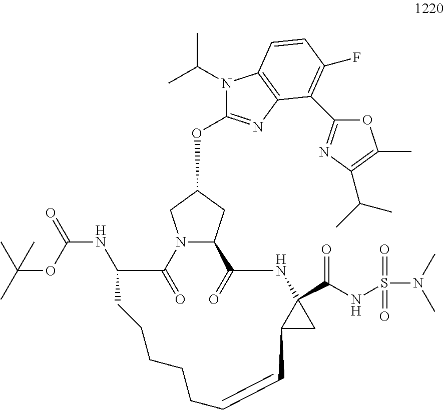

Yielded 13 mg (62%) of compound 1220 as a white solid after flash column chromatography. ¹H NMR (500 MHz, CDCl₃) δ ppm 9.89 (br. s., 1H) 7.21 (dd, J=8.62, 3.89 Hz, 1H) 6.97 (dd, J=10.83, 9.00 Hz, 1H) 6.74 (br. s., 1H) 5.98 (br. s., 1H) 5.70-5.80 (m, 1H) 4.98-5.08 (m, 2H) 4.45-4.62 (m, 3H) 4.24-4.33 (m, 1H) 3.98 (dd, J=11.67, 2.67 Hz, 1H) 2.94-3.03 (m, 1H) 2.87 (s, 6H) 2.73-2.83 (m, 1H) 2.64-2.73 (m, 1H) 2.51-2.63 (m, 1H) 2.41 (s, 3H) 2.24 (q, J=7.99 Hz, 1H) 1.83-1.95 (m, 2H) 1.73-1.83 (m, 1H) 1.56-1.63 (m, 2H) 1.50 (d, J=6.87 Hz, 6H) 1.43-1.50 (m, 2H) 1.36 (s, 9 H) 1.34 (dd, J=7.02, 1.53 Hz, 6H) 1.19-1.31 (m, 4H). LC-MS: purity 100% (UV), t_{R} 5.20 min m/z [M+H]⁺ 871.75 (MET/CR/1416).

### 2.43 Synthesis of Compounds 1221 and 1222

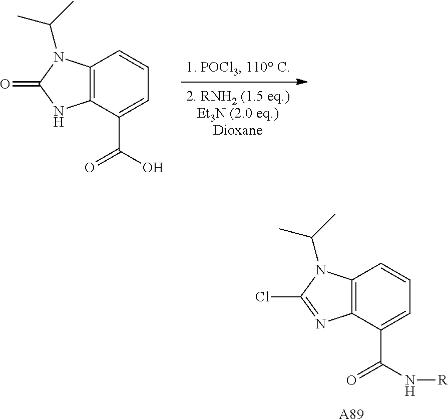

Preparation of precursors 2-Chloro-1-isopropyl-benzimidazole-4-carboxylic acid (4-isopropyl-thiazol-2-yl)-amide A89a and 2-Chloro-1-isopropyl-benzimidazole-4-carboxylic acid (4-isopropyl-thiazol-2-yl-methyl)-amide A89b:

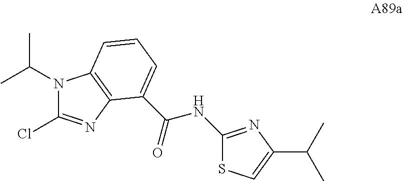

1-Isopropyl-2-oxo-2,3-dihydro-benzimidazole-4-carboxylic acid (100 mg, 0.454 mmol, 1.0 eq.) was dissolved in phosphorous oxychloride (2 mL) and the reaction mixture heated at 110° C. for 15 hours. The solvent was removed in vacuo. The residue was taken up in anhydrous dioxane (2 mL) and triethylamine (0.126 mL, 0.908 mmol, 2.0 eq.) was added as a single portion. 2-amino-4-isopropyl-thiazole (72 mg, 0.476 mmol, 1.05 eq.) was diluted with anhydrous dioxane (1 mL) and the resulting solution added dropwise to the reaction mixture and stirring continued at ambient temperature for a further 2 hours. The reaction mixture was diluted with water (4 mL) and extracted with ethyl acetate (3×10 mL). The combined organic extracts were washed with brine (10 mL), dried over magnesium sulfate, filtered and the solvent removed in vacuo to give 124 mg (75% yield) of compound A89a as a light yellow solid. ¹H NMR (500 MHz, CDCl₃) δ ppm 12.58 (br. s., 1H) 8.22 (d, J=7.78 Hz, 1H) 7.72 (d, J=8.24 Hz, 1H) 7.42 (t, J=8.01 Hz, 1H) 6.58 (s, 1H) 5.00 (spt, J=6.94 Hz, 1H) 3.00-3.09 (m, 1H) 1.67-1.73 (m, 3H) 1.30-1.35 (m, 3H) 1.17-1.30 (m, 6H). LC-MS: purity 60% (UV), t_{R} 2.56 min m/z [M+H]⁺ 363.40 (MET/CR/1278).

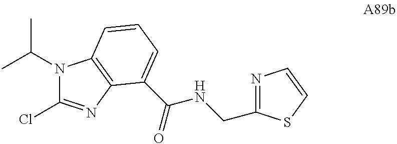

The above method was used to prepare compound A89b, which yielded 95 mg (62%) as a white solid after flash column chromatography. ¹H NMR (500 MHz, CDCl₃) δ ppm 12.58 (br. s., 1H) 8.22 (d, J=7.78 Hz, 1H) 7.72 (d, J=8.24 Hz, 1H) 7.42 (t, J=8.01 Hz, 1H) 6.58 (s, 1H) 5.00 (spt, J=6.94 Hz, 1H) 3.00-3.09 (m, 1H) 1.67-1.73 (m, 3H) 1.30-1.35 (m, 3H) 1.17-1.30 (m, 6H). LC-MS: purity 72% (UV), t_{R} 1.94 min m/z [M+H]⁺334.95 (MET/CR/1278).

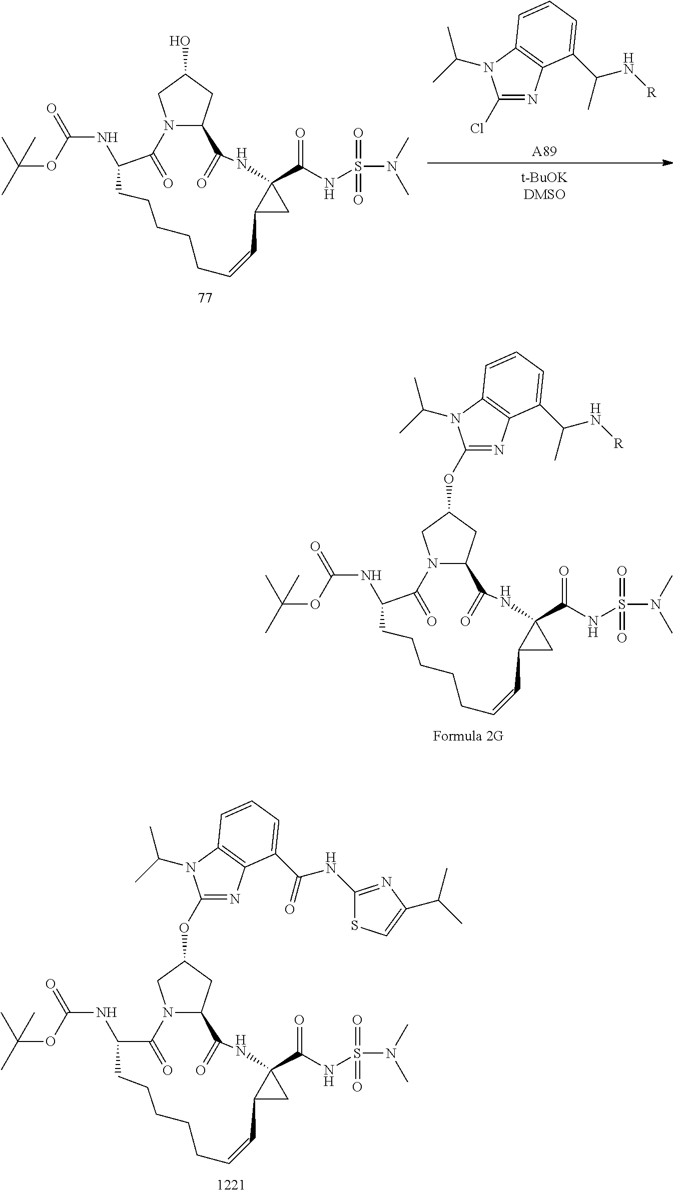

Compounds 1221 and 1222 were prepared according to the method for preparing compound 1201 as described in section 2.36, using precursor compounds A89a and A89b, respectively. Yielded 26 mg (18%) of compound 1221 as a beige solid after preparative HPLC. ¹H NMR (500 MHz, CDCl₃) δ ppm 12.81 (br. s., 1H) 9.94 (br. s., 1H) 8.11 (d, J=7.63 Hz, 1H) 7.41-7.57 (m, 1H) 7.27-7.32 (m, 1H) 6.63-6.82 (m, 1H) 6.46-6.61 (m, 1H) 5.88-6.10 (m, 1H) 5.67-5.84 (m, 1H) 4.98-5.14 (m, 1H) 4.81-4.98 (m, 1 H) 4.54-4.73 (m, 2H) 4.14-4.30 (m, 1H) 3.97-4.14 (m, 1H) 2.95-3.11 (m, 1H) 2.88 (s, 6H) 2.75-2.86 (m, 2H) 2.52-2.69 (m, 1H) 2.19-2.32 (m, 1H) 1.83-1.98 (m, 2H) 1.72-1.83 (m, 1H) 1.56 (d, J=6.71 Hz, 6H) 1.45-1.53 (m, 3H) 1.36-1.45 (m, 3H) 1.33 (d, J=5.65 Hz, 6H) 1.27-1.31 (m, 2H) 1.23 (br. s., 9H) 1.05-1.14 (m, 1H). LC-MS: purity 100% (UV), t_{R} 4.77 min m/z [M+H]⁺ 998.32 (MET/CR/1426).

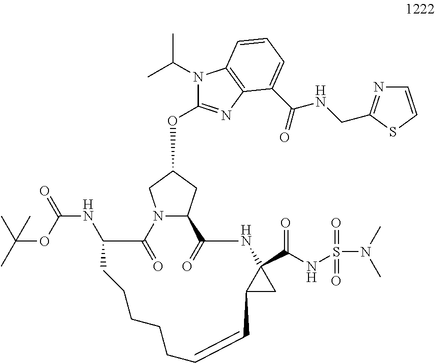

Yielded 23 mg (17%) of compound 1222 as an off-white solid after preparative HPLC. ¹H NMR (500 MHz, CDCl₃) δ ppm 10.04-10.41 (m, 2H) 8.07 (d, J=7.78 Hz, 1H) 7.77-7.87 (m, 1H) 7.38-7.46 (m, 2H) 7.22-7.27 (m, 1H) 6.79-6.97 (m, 1H) 5.81-5.92 (m, 1H) 5.75 (q, J=9.10 Hz, 1H) 5.25 (dd, J=15.72, 6.10 Hz, 1H) 4.96-5.12 (m, 3H) 4.49-4.68 (m, 3H) 4.24-4.34 (m, 1H) 3.94-4.23 (m, 1H) 2.90 (s, 6H) 2.74-2.83 (m, 1H) 2.64-2.74 (m, 1H) 2.48-2.62 (m, 1H) 2.25 (q, J=8.85 Hz, 1H) 1.86-1.97 (m, 3H) 1.77-1.85 (m, 1H) 1.53 (dd, J=6.79, 2.98 Hz, 6H) 1.45-1.51 (m, 3H) 1.38-1.44 (m, 2H) 1.34 (s, 9H) 1.24-1.31 (m, 1H) 1.19-1.23 (m, 1H). LC-MS: purity 98% (UV), t_{R} 3.96 min m/z [M+H]⁺ 870.29 (MET/CR/1426).

### Example 3

### Quinoxaline Analogs

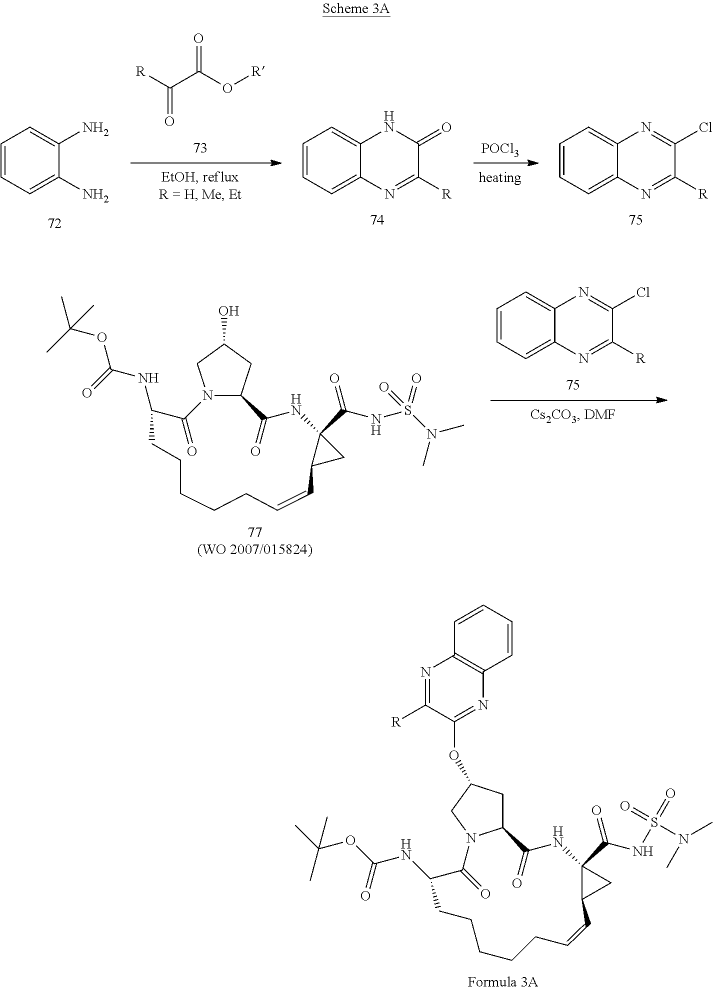

### 3.1 Synthesis of Precursor Compound 74

Method A: from acid

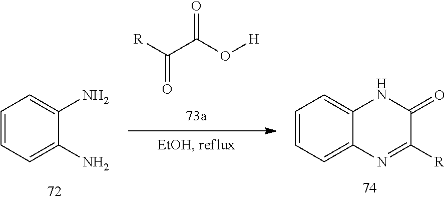

A mixture of o-phenylenediamine 72 (1 eq.) and acid 73a (1 eq.) in ethanol was refluxed for 16 hours under nitrogen atmosphere. The precipitate formed during this time was collected and washed with ethanol to give compound 74 as a solid.

The following precursor was prepared using Method A:

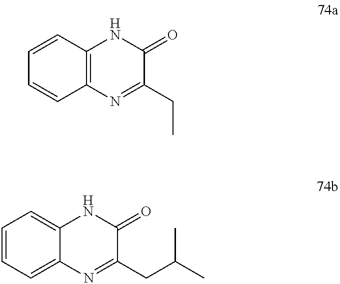

For compound 74b, 4-methyl-2-oxopentanoic acid is converted from sodium salt:

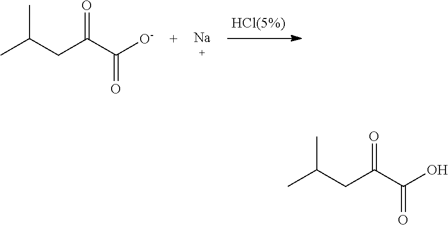

To a solution of sodium salt of 4-methyl-2-oxopentanoic acid (370 mg, 2.43 mmol) in 3 mL of water was added carefully aq. HCl (5%), adjust to pH=6. The mixture was extracted by ethyl acetate (20 mL×3), the organic layers were combined, washed by brine, dried over anhydrous sodium sulfate, solvent was removed under reduced pressure to gave the acid (250 mg, 79%), it is used directly in the next step.

Method B: from ester

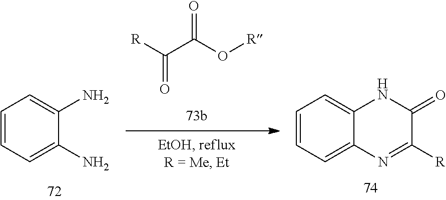

A mixture of o-phenylenediamine 72 (1 eq.) and ester 73b (1 eq.) in ethanol was stirred at room temperature under nitrogen atmosphere. After the starting material was consumed, the precipitate formed during this time was collected and washed with ethanol to give intermediate 74 as a solid.

The following precursor was prepared using Method B:

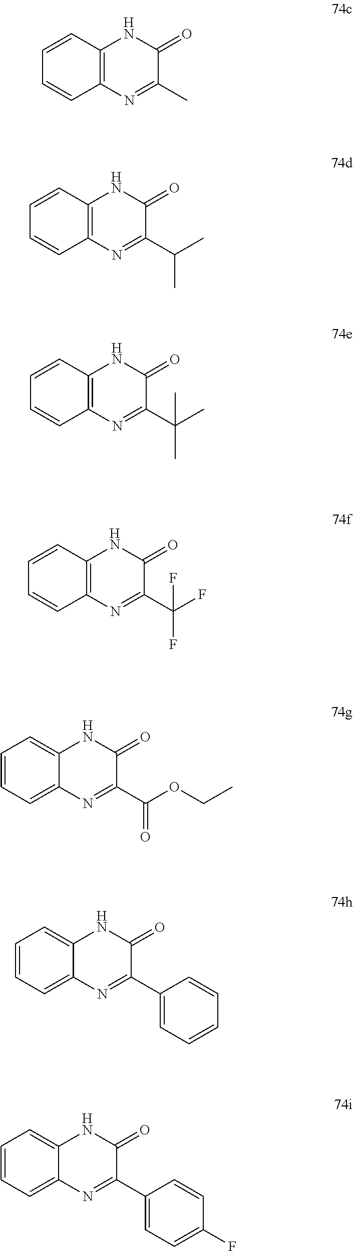

### 3.2 Synthesis of Precursor Compound 75

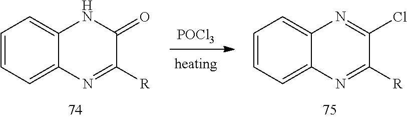

A mixture of compound 74 and POCl₃ was heated to reflux at 120° C. After the material was consumed, the reaction mixture was cooled to r.t. and then taken up with ice-water. The aqueous layer was neutralized with saturated aq. NaHCO₃, extracted with EtOAc. The extracts were washed with water and brine, dried over anhydrous sodium sulfate. The solvent was removed in vacuo and the residue was purified by column chromatography using PE:EA=10:1 as eluent to give compound 75

This method was adopted for preparation of following chlorides 75:

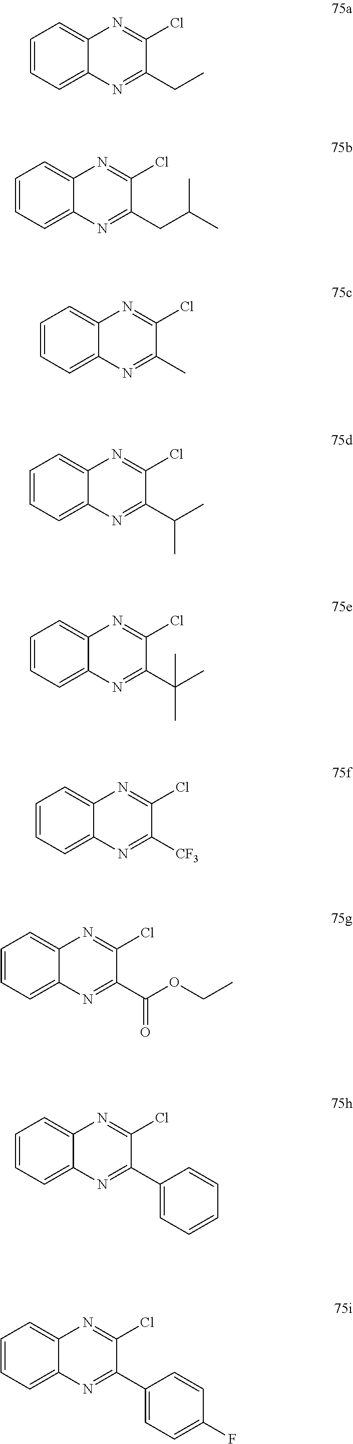

### 3.3 Synthesis of Macrocyclic Compounds of Formula 3A

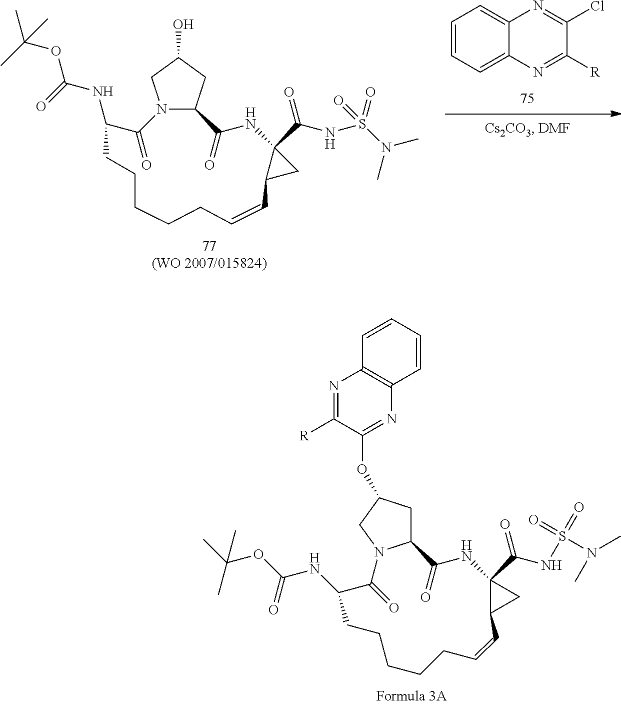

A flask was charged with compound 77 (1 eq.) and DMF. The mixture was purged with nitrogen for three times. Cesium carbonate (6 eq.) was added and keep stirring for 10 minutes at r.t. After that, compound 75 (1.3 eq.) was added. The reaction mixture was heated at 60˜70° C. for 12 hrs. After the material was consumed, the reaction was cooled to r.t and water was added, the mixture was acidified with aq. HCl (1N) to pH=5-6, and then extracted with ethyl acetate, washed with water and brine. The organic layer was dried over anhydrous sodium sulfate and the solvent was removed. The residue was purified with prep-HPLC to give the title compound.

This method was adopted for preparation of compounds 301-308.

TABLE 12
Compounds prepared using Scheme 3A.

| Compound | Structure | Yield |
|---|---|---|
| 301 | 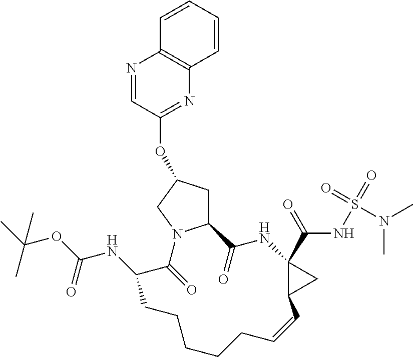 | 64.2 mg, 29%. MS (ESI) m/z (M + H)⁺ 700.1 |
| 302 | 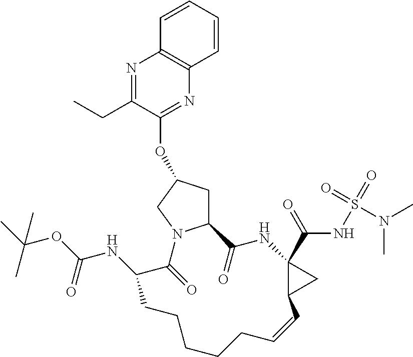 | 5.3 mg, 11%. MS (ESI) m/z (M + H)⁺ 728.2 |
| 303 | 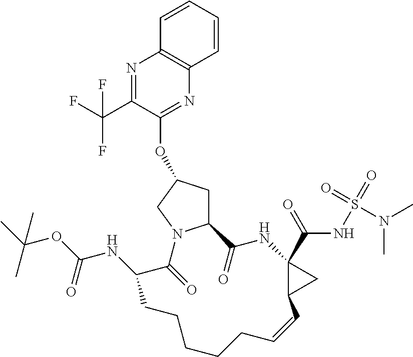 | 8.8 mg, 13%. MS (ESI) m/z (M + Na)⁺ 790.1 |
| 304 | 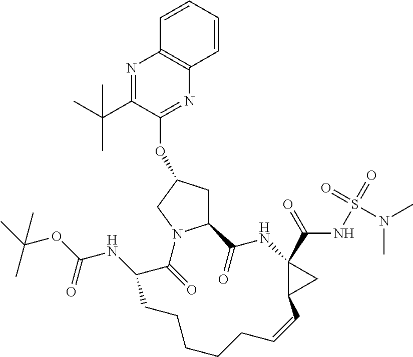 | 7.6 mg, 12%. MS (ESI) m/z (M + H)⁺ 756.2 |
| 305 | 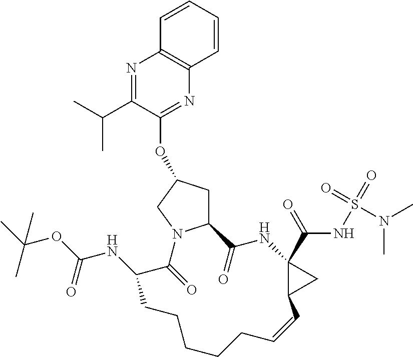 | 71.1 mg, 22%. MS (ESI) m/z (M + H)⁺ 742.2 |
| 306 | 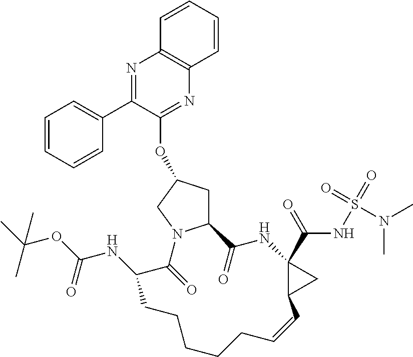 | 9.1 mg, 14%. MS (ESI) m/z (M + Na)⁺ 798.2 |
| 307 | 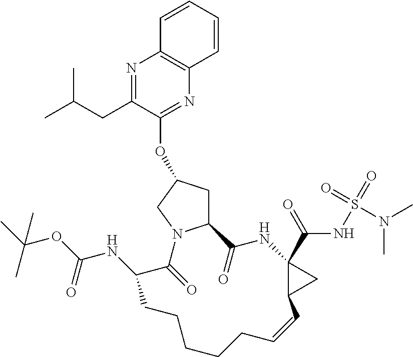 | 9.7 mg, 15%. MS (ESI) m/z (M + H)⁺ 755.9 |
| 308 | 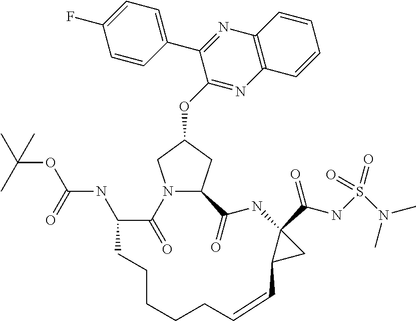 | 9.9 mg, 14%. MS (ESI) m/z (M + H)⁺ 794.0 |

### 3.4 Synthesis of Compound 309

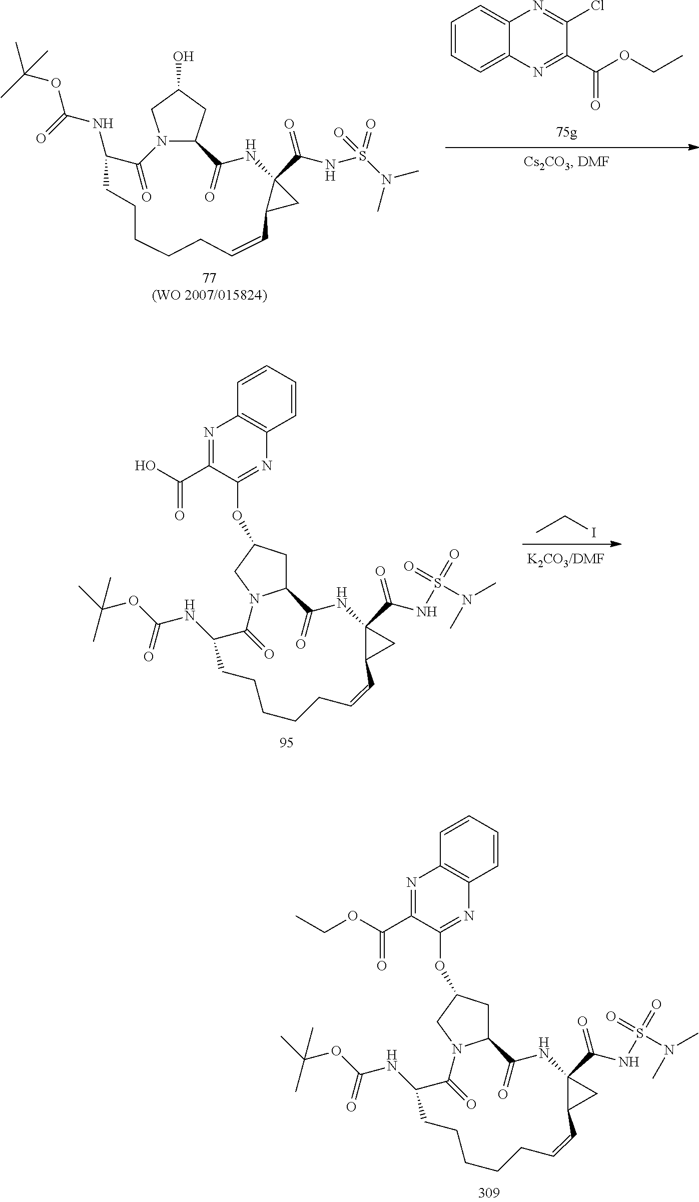

To a solution of compound 95 (20 mg, 0.0269 mmol) in 0.5 mL of DMF was added K₂CO₃ (3.7 mg, 0.0269 mmol) and iodoethane (4 mg, 0.0269 mmol). The mixture was stirred for 12 h, the reaction was monitored by LCMS. After completion of the reaction, the mixture was extracted by ethyl acetate (20 mL×3), the organic layers were combined, washed by brine, dried over anhydrous sodium sulfate, solvent was removed under reduced pressure, the crude product was purified by prep-HPLC to afford compound 309. 8.4 mg, 40.6%. MS (ESI) m/z (M+Na)⁺ 794.2.

### 3.5 Synthesis of Macrocyclic Compounds of Formula 3B

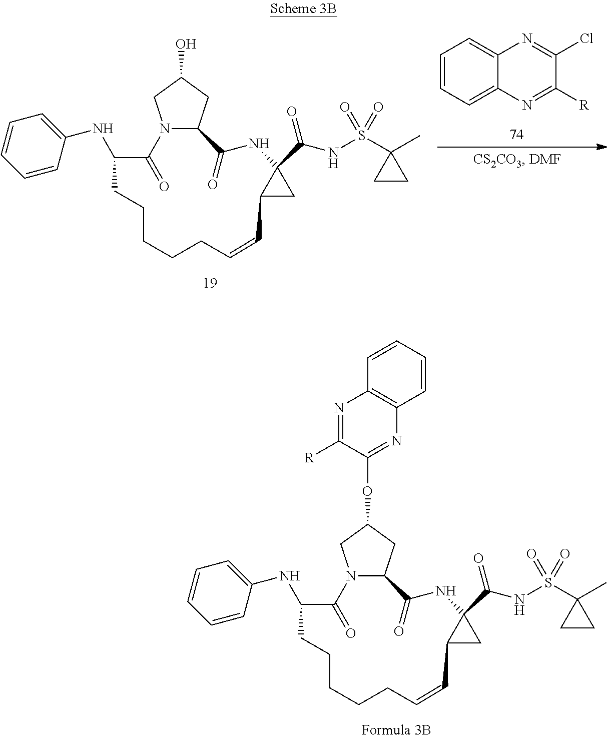

Compound 19 was synthesized according to Scheme 2A. A flask was charged with compound 19 (1 eq.), Cs₂CO₃ (6.0 eq.) and DMF (2 mL). The mixture was stirred under nitrogen at room temperature for 20 min. Compound 74 (1.2 eq., 73 mg, 0.41 mmol) was added. The reaction mixture was stirred for 12 hrs. The LCMS shows reaction completion, the reaction was quenched with ice-water, acidified with aq. HCl (1 N) to pH=5-6, extracted with EtOAc, dried over sodium sulfate, concentrated to give crude product Formula 3B, it was purified with prep-HPLC to give desired product.

This method was adopted for preparation of compounds 310-312.

TABLE 13
Compounds prepared according to Example 3.5.

| Compound | Structure | Yield |
|---|---|---|
| 310 | 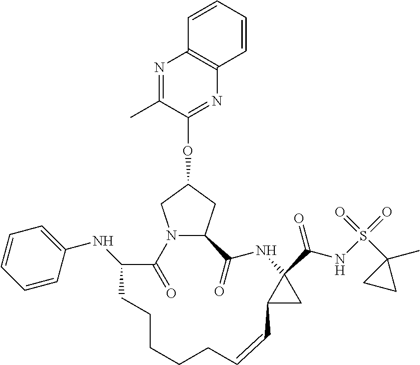 | 121.8 mg, 51%. MS (ESI) m/z (M + H)⁺ 701.1 |
| 311 | 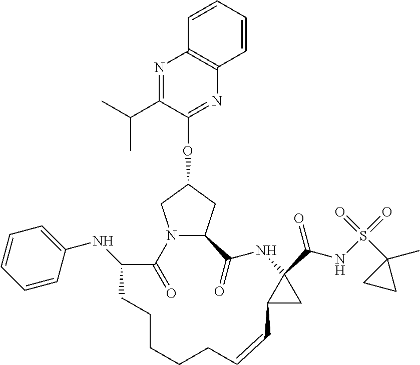 | 5.2 mg, 13%. MS (ESI) m/z (M + H)⁺ 729.3 |
| 312 | 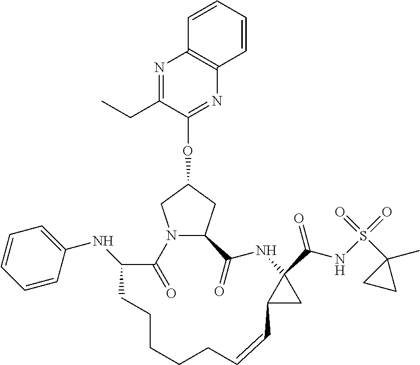 | 6.7 mg, 13%. MS (ESI) m/z (M + Na)⁺ 715.0 |

### Example 4

### 4.1 Synthesis of Precursor Compound 82

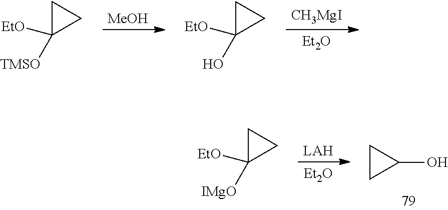

A solution of 1-ethoxy-1-trimethylsiloxycyclopropane (17.4 g, 0.1 mol) in 150 mL of methanol was stirred at room temperature for 8 h. The solvent was removed slowly at room temperature on a rotary evaporator and a short-path distillation yielded the pure 1-ethoxycyclopropanol (5.5 g, 54%).

To a solution of 1-ethoxycyclopropanol (2 g, 20 mmol) in anhydrous diethyl ether (40 mL) was added a solution of methylmagnesium iodide (3.0 M in diethyl ether, 6.7 mL, 20 mmol) at 0° C. with an ice-bath. A gas, presumably methane, evolved, while a white suspension was formed. To the stirred suspension was added lithium aluminium hydride (1.14 g, 30 mmol) in portions. After the addition was over, the reaction mixture was brought to room temperature (30 min) and then maintained under reflux for 2 h with an oil bath. The mixture was then cooled to room temperature and hydrolyzed by addition of wet sodium sulfate. The ether layer was separated, washed with water (1 mL), dried over sodium sulfate, distilled ether and obtained the residue as a colorless oil (compound 79), it was used directly in next step. ¹H NMR (400 MHz, CDCl₃) δ3.43 (m, 1H), 2.38 (s, 1H), 0.50 (m, 4H).

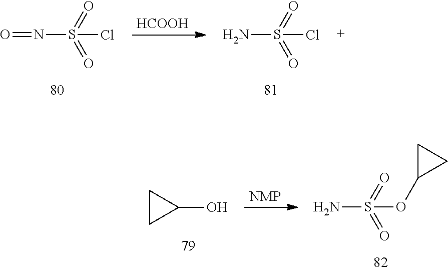

A flask with chlorosulfonyl isocyanate 80 (1.8 mL) was cooled to 0° C., formic acid (0.77 mL) was added dropwise with rapid stirring with gas evolution observed, upon completion addition of the formic acid, the reaction was let warm to room temperature, the mixture (compound 81) was stirred at this temperature for 2 hours.

To the reaction mixture of compound 81 was added compound 79 in 5 mL of NMP at 0° C., the reaction mixture was let warm to room temperature, after 3 hours stirring, the reaction mixture was poured into ice brine, then the mixture was extracted with EtOAc, the organic layer was separated, washed by brine, dried over anhydrous Na₂SO₄, the solvent was removed under reduced pressure, the brown solution (400 mg) of compound 82 was used directly in the next step.

### 4.2 Synthesis of Macrocyclic Compound 401

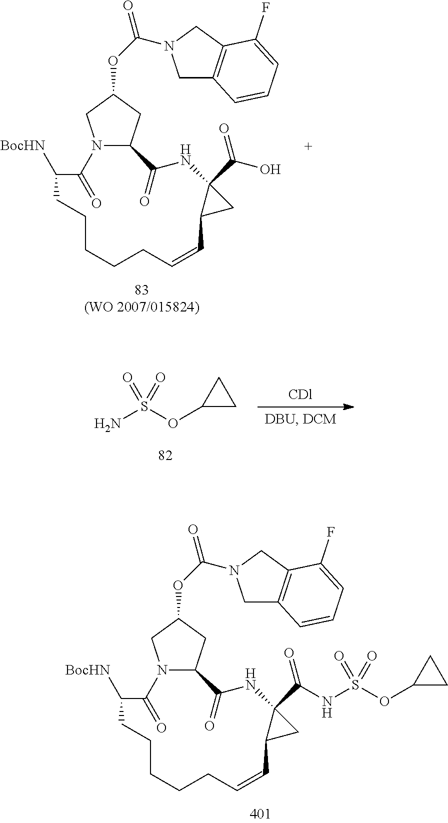

Compound 83 was obtained according to methods described in PCT Publication No. WO 2007/015824, which is incorporated herein by reference in its entirety. To a solution of compound 83 (400 mg, 0.64 mmol.) in anhydrous dichloromethane (20 mL) was added CDI (415.2 mg, 2.56 mmol). The resulting mixture was stirred at 40-50° C. for 4 h, then compound 82 (400 mg) and DBU (0.39 mL, 2.55 mmol) was added, the resulting mixture was stirred at room temperature for another 12 h and the reaction was monitored by LCMS. After completion of the reaction, the solvent was removed and the crude was purified by prep-HPLC to give compound 401 as the white solid. (25 mg, 5.0%). MS (ESI) m/z (M+H)⁺769.8.

### Example 5

### Purine Analogs

### 5.1 Synthesis of Precursor Compound 8-Chloro-9-isopropyl-purine

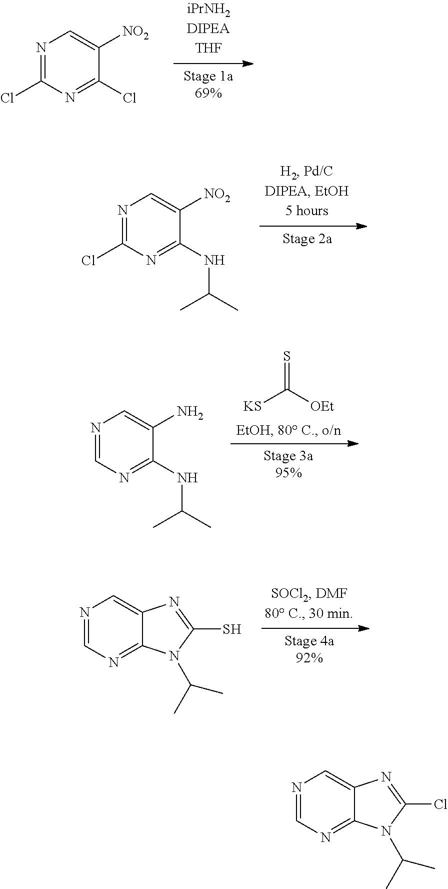

Stage 1a—2-Chloro-4-isopropylamino-5-nitro-pyrimidine: 2,4-Dichloro-5-nitro-pyrimidine (11.9 g, 61.30 mmol, 1.0 eq.) and tetrahydrofuran (180 mL) were charged into a 500 mL round bottom flask placed in ice/water bath. Diisopropylethylamine (75 mL, 0.429 mol, 7.0 eq.) was added portion wise. Isopropylamine (5.22 mL, 61.30 mmol, 1.0 eq.) was diluted with tetrahydrofuran (35 mL). The solution was added dropwise, over 15 minutes, to the reaction mixture. Stirring was continued for a further 5 minutes and checked by LCMS to show the reaction was complete. The reaction mixture was filtered and the solvent removed under vacuum. The residue was taken up in ethyl acetate (130 mL) and the organic phase washed with 10% aqueous citric acid (2×55 mL). The organic phase was dried over sodium sulfate, filtered and the solvent removed under vacuum to give a dark oil (12.9 g). The oil was purified by flash column chromatography using a heptanes: ethyl acetate gradient (from neat heptanes to 10% ethyl acetate in heptanes). After combining the relevant fractions and removing the solvent under vacuum, 8.37 g (69%) of the title compound was isolated as a yellow oil. ¹H NMR (250 MHz, CDCl₃) δ ppm 9.03 (s, 1H) 8.24 (br. s., 1H) 4.43-4.64 (m, 1H) 1.34 (d, J=6.55 Hz, 6H). LC-MS: purity 99% (UV), m/z [M+H]⁺ 216.90, 1.90 min (MET/CR/1278).

Stage 2a—4-Isopropylamino-5-amino-pyrimidine: 2-Chloro-4-isopropylamino-5-nitro-pyrimidine (6.28 g, 29.0 mmol, 1.0 eq.) was diluted in ethanol (200 mL) into a 500 mL round bottom flask fitted with a 3 way tap. Diisopropylethylamine (30.3 mL, 174.0 mmol, 6.0 eq.) was added dropwise. 10% Pd/C (50% wet, 1.25 g, 10 wt % catalyst) was added as a single portion. The reaction mixture was degassed with nitrogen/vacuum cycle (3 times) then flushed with hydrogen gas. The reaction mixture was then stirred under a hydrogen gas atmosphere for 15 hours. The catalyst was removed by filtration and the filtrate used directly in stage 3a. LC-MS: purity 83% (UV), m/z [M+H]⁺153.00, 1.32 min (MET/CR/1278).

Stage 3a—8-Thio-9-isopropyl-purine: to the ethanol filtrate from stage 2a was added potassium ethyl xanthate (9.30 g, 58 mmol, 2.0 eq.). The reaction mixture was heated at 80° C. for 15 hours by when LCMS analysis showed the reaction to be completed. The reaction mixture was filtered and the solvent removed under vacuum. Water (100 mL) was added and 1M hydrochloric acid was added until pH=4. The solution was extracted with chloroform/methanol (7:3, 2×300 mL) and the solvent removed under vacuum to give 5.35 g (95%) of the title compound as a pale brown solid which was used in the next step without further purification. ¹H NMR (500 MHz, CDCl₃) δ ppm 8.85 (s, 1H) 8.62 (s, 1H) 5.40 (spt, J=6.94 Hz, 1H) 2.26-3.08 (m, 1H) 1.69 (d, J=6.87 Hz, 6H). LC-MS: purity 92% (UV), m/z [M+H]⁺ 194.90, 1.52 min (MET/CR/1278).

Stage 4a—8-Chloro-9-isopropyl-purine: 8-Thio-9-isopropyl-purine (420 mg, 2.61 mmol, 1.0 eq.), thionyl chloride (3 mL) and N,N-dimethylformamide (0.2 mL) were charged into a 10 mL flask. The reaction mixture was heated at (80° C.) for 30 minutes. The solvent was removed under vacuum and the residue azeotroped twice with toluene (10 mL) to give 391 mg (92%, corrected for solvent contents) of the title compound as a beige solid. Compound used in next stage without further purification. ¹H NMR (500 MHz, CDCl₃) ppm 8.73 (s, 1H) 8.47 (br. s., 1H) 4.80 (spt, J=6.88 Hz, 1H) 1.62 (d, J=7.02 Hz, 6H). LC-MS: purity 73% (UV), m/z [M+H]⁺ 196.90, 1.51 min (MET/CR/1278).

### 5.2 Synthesis of Macrocyclic Compounds 501, 502 and 503

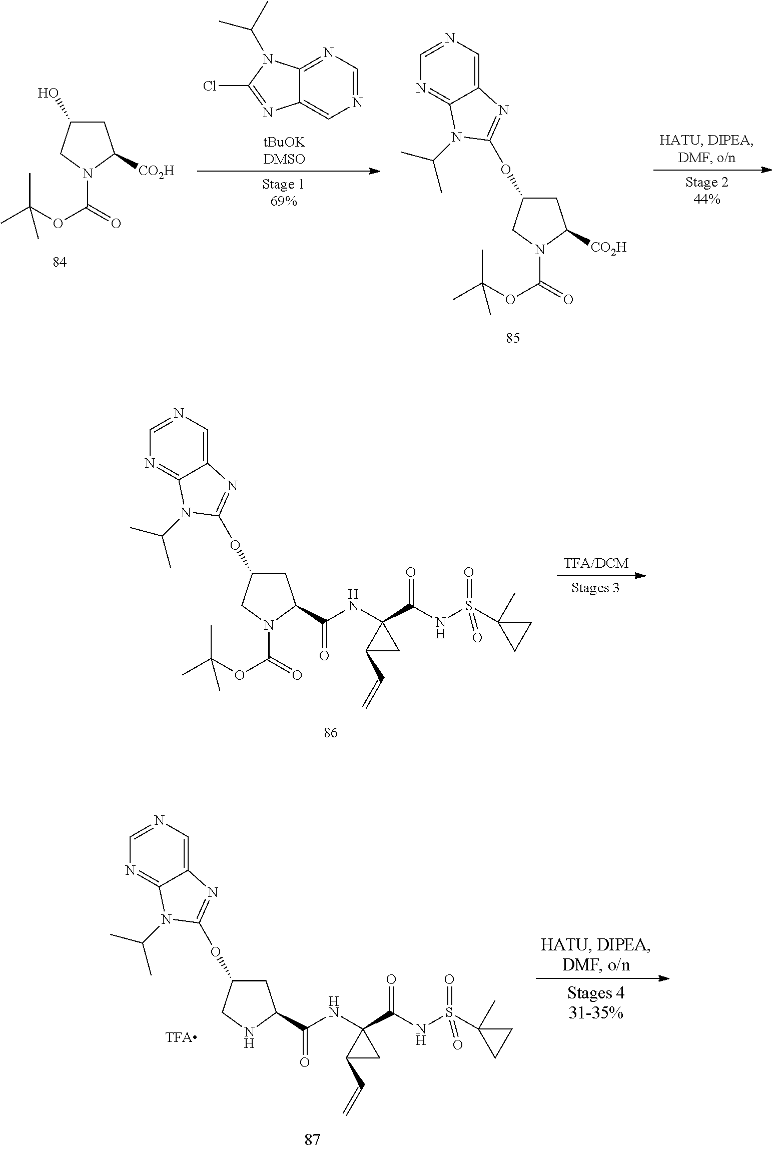

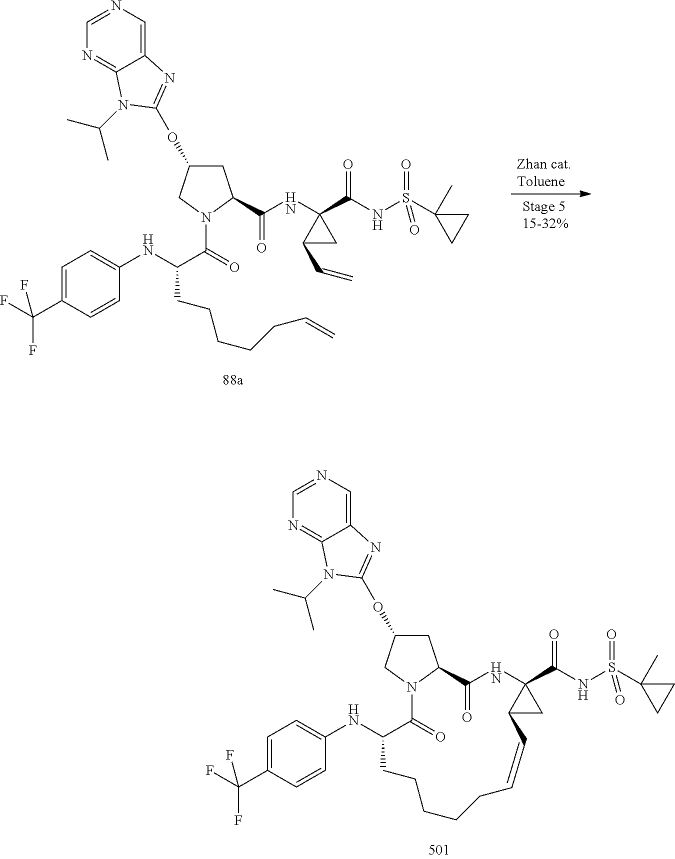

Stage 1—(2S,4R)-1-(tert-Butoxycarbonylamino)-4-(9-isopropyl-purine-2-oxy)-proline (85): (2S,4R)-1-(tent-Butoxycarbonylamino)-4-hydroxy-proline (84) (500 mg, 2.17 mmol, 1.0 eq.) and dimethylsulfoxide (7.5 mL) were charged into a 25 mL round bottom flask. Potassium tert-butoxide (509 mg, 4.54 mmol, 2.1 eq.) was added portionwise over 10 minutes at ambient temperature. The reaction mixture was stirred for a further 1 hour at ambient temperature. 8-Chloro-9-isopropyl-purine (425 mg, 2.17 mmol, 1.0 eq.) was added portionwise and the stirring was continued at 50° C. for 15 hours by which time LCMS analysis of the reaction mixture showed ˜35% (UV) of remaining chloropurine. Potassium tert-butoxide (242 mg, 2.17 mmol, 1.0 eq.) was added and the reaction mixture stirred at 50° C. for a further 15 hours. LCMS analysis of the reaction mixture showed the reaction to be completed. The reaction mixture was diluted with methanol (7 mL) and stirred for 30 minutes. The reaction mixture was left to cool to ambient temperature and was diluted with ethyl acetate (10 mL) and water (4 mL). The aqueous phase was acidified to pH=3 with 1M hydrochloric acid and extracted with ethyl acetate (3×8 mL). The organic extracts were combined, washed with brine (10 mL), dried over sodium sulfate, filtered and the solvent removed under vacuum to give 910 mg (69%, 583 mg corrected for solvent content) of the title compound 85 as a sticky gum which contained dimethyl sulfoxide (36% w/w by ¹H NMR). Product was used in the next step without further purification. ¹H NMR (500 MHz, CDCl₃) δ ppm 8.82 (s, 1H) 8.78 (br. s., 1H) 5.76 (br. s., 1H) 4.88 (dt, J=13.66, 6.75 Hz, 1 H) 4.42-4.66 (m, 1H) 3.81-3.96 (m, 1H) 2.65-2.85 (m, 1H) 2.48-2.65 (m, 1H) 1.57 (d, J=6.87 Hz, 6H) 1.53 (d, J=7.32 Hz, 1H) 1.45 (d, J=7.17 Hz, 9H) 1.35-1.43 (m, 1H). LC-MS: purity 100% (UV), m/z [M+H]⁺ 392.10, 1.62 min (MET/CR/1981).

Stage 2—Compound 86: (2S,4R)-1-(tert-Butoxycarbonylamino)-4-(9-isopropyl-purine-2-oxy)-proline (85) (582 mg, 1.49 mmol, 1.0 eq.) and N,N-dimethylformamide (12 mL) were charged into a 50 mL round bottom flask under nitrogen. HATU (737 mg, 1.94 mmol, 1.3 eq.) and diisopropylethylamine (1.6 mL, 8.93 mmol, 6.0 eq.) were added at 0° C. and the reaction mixture stirred at ambient temperature for a further 30 minutes. (1R,2S)-1-Amino-2-vinyl-cyclopropane-1-carbonyl-(1′-methyl)cyclopropane-sulfonamide hydrochloride salt (364 mg, 1.49 mmol, 1.0 eq.), previously dissolved in N,N-dimethylformamide (6 mL) was added dropwise over 15 minutes at 0° C. and stirring was continued for 21 hours at ambient temperature. Monitoring the reaction extent by LCMS showed near complete consumption of the starting material. The solvent was removed under vacuum and the residue partitioned between water (60 mL) and ethyl acetate (60 mL). The phases were separated and the organic phase washed with water (60 mL) and brine (60 mL), dried over sodium sulfate, filtered and the solvent removed under vacuum. The residue was purified by flash column chromatography, using a methanol: dichloromethane gradient (from neat dichloromethane to 2% methanol in dichloromethane). After combining the relevant fractions and solvent removal, 406.0 mg (44%) of the title compound 86 was isolated as a brown oil. ¹H NMR (500 MHz, CDCl₃) δ ppm 9.83 (br. s., 1H) 8.68 (s, 1H) 8.64 (br. s., 1 H) 8.03 (s, 1H) 5.54-5.70 (m, 2H) 4.95-5.04 (m, 1H) 4.75 (dt, J=13.66, 6.75 Hz, 1H) 4.30 (t, J=8.01 Hz, 1H) 3.69-3.90 (m, 1H) 2.36-2.55 (m, 2H) 2.08-2.16 (m, 1H) 1.73-1.89 (m, 1H) 1.51-1.58 (m, 1H) 1.48 (br. s., 1H) 1.44 (dd, J=6.71, 2.75 Hz, 6H) 1.40 (s, 3H) 1.36 (d, J=5.34 Hz, 12H) 0.67-0.83 (m, 2H). LC-MS: purity 84% (UV), m/z [M+H]⁺618.15, 1.71 min (MET/CR/1981).

Stage 3—Compound 87: Stage 2 intermediate compound 86 (406 mg, 0.657 mmol, 1.0 eq.) and dichloromethane (13 mL) were charged into a 50 mL round bottom flask and the reaction mixture cooled to 0° C. Trifluoroacetic acid (2.3 mL) was added dropwise over 5 minutes and the dark orange reaction mixture stirred at ambient temperature for 1 hour. LCMS analysis showed full consumption of the starting material. The solvent was removed under vacuum and the residue dried further under high vacuum for 4 hours to give 420 mg (100%) of the title compound 87 as a brown solid. The product was used in the next step without further purification. LC-MS: purity 89% (UV), m/z [M+H]⁺ 518.05, 1.28 min (MET/CR/1278).

Stage 4—Synthesis of Intermediates 88a, 88b and 88c:

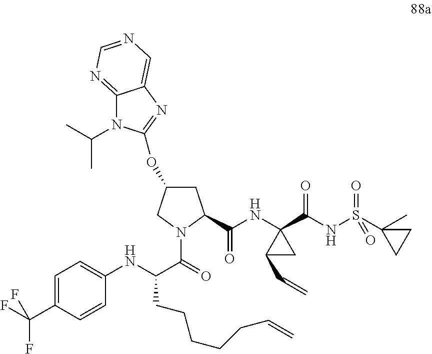

Stage 3 intermediate compound 87 (TFA salt, 127 mg, 0.202 mmol, 1.0 eq.) and N,N-dimethylformamide (2 mL) were charged into a 10 mL round bottom flask under nitrogen. HATU (100 mg, 0.263 mmol, 1.3 eq.) and diisopropylethylamine (0.211 mL, 1.212 mmol, 6.0 eq.) were added at 0° C. and the reaction mixture stirred at ambient temperature for an additional 15 minutes. (2S)-2-(4-trifluoromethyl-phenylamino)-non-8-enoic acid (64 mg, 0.202 mmol, 1.0 eq.) was added as a single portion and stirring was continued at ambient temperature for an additional 15 hours. The solvent was removed under vacuum and the residue partitioned between ethyl acetate (6 mL) and water (6 mL). The organic phase was further washed with water (2×3 mL) and brine (6 mL), dried over sodium sulfate, filtered and concentrated to dryness. The residue was purified by flash column chromatography, using ethyl acetate as eluent. After combining the relevant fractions the solvent was removed under vacuum to give 58 mg (35%) of the title compound 88a as a yellow oil. ¹H NMR (500 MHz, CDCl₃) δ ppm 10.05 (br. s., 1H) 8.75-8.86 (m, 2H) 6.54 (d, J=8.39 Hz, 2H) 5.90 (br. s., 1H) 5.67-5.83 (m, 2H) 5.24 (d, J=16.94 Hz, 1H) 5.12 (d, J=10.38 Hz, 1H) 4.98 (dd, J=17.09, 1.37 Hz, 1H) 4.92 (d, J=10.22 Hz, 1H) 4.83 (m, J=13.73, 6.87, 6.87, 6.87, 6.87 Hz, 1H) 4.49 (t, J=8.09 Hz, 1H) 4.14-4.23 (m, 2H) 4.16 (br. s., 1H) 4.07-4.11 (m, 1H) 2.55-2.70 (m, 2H) 2.09 (q, J=8.80 Hz, 1H) 1.96-2.04 (m, 3H) 1.73-1.87 (m, 2H) 1.62-1.74 (m, 3H) 1.51-1.58 (m, 3H) 1.50 (d, J=7.32 Hz, 6 H) 1.41-1.47 (m, 3H) 1.29-1.41 (m, 6H) 0.91 (d, J=3.36 Hz, 1H) 0.80-0.88 (m, 1H). LC-MS: purity 100% (UV), t_{R} 2.18 min m/z [M+H]⁺ 815.35 (MET/CR/1981).

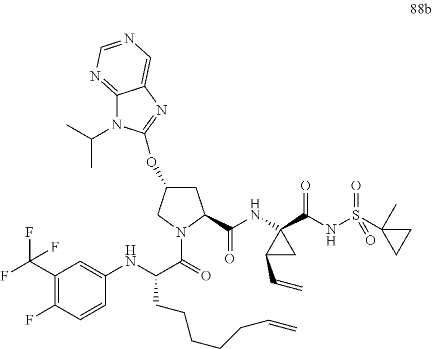

Compound 88b was prepared as described for Compound 88a starting from compound 87 (207 mg, 0.328 mmol). Yield compound 88b, 98 mg (36%) as a yellow oil. ¹H NMR (500 MHz, CDCl₃) δ ppm 10.10 (s, 1H) 8.81 (d, J=17.70 Hz, 2H) 7.00-7.15 (m, 1H) 6.91 (t, J=9.31 Hz, 1H) 6.70-6.77 (m, 1H) 6.68 (dd, J=5.34, 2.90 Hz, 1H) 5.90 (br. s., 1H) 5.71-5.86 (m, 2H) 5.24 (d, J=17.09 Hz, 1H) 5.14 (d, J=10.38 Hz, 1H) 4.99 (d, J=17.09 Hz, 1H) 4.94 (d, J=10.07 Hz, 1H) 4.76-4.86 (m, 1H) 4.74 (br. s., 1H) 4.42 (t, J=8.32 Hz, 1H) 4.08-4.13 (m, 2H) 4.06 (d, J=5.65 Hz, 1H) 2.64 (d, J=7.17 Hz, 2H) 2.06-2.11 (m, 2H) 2.01-2.05 (m, 2H) 1.72-1.85 (m, 3H) 1.70 (br. s., 2H) 1.58 (d, J=7.63 Hz, 1H) 1.53 (d, J=6.87 Hz, 3H) 1.48-1.51 (m, 6H) 1.43-1.48 (m, 2H) 1.31-1.43 (m, 5 H). LC-MS: purity 100% (UV), m/z [M+H]⁺ 833.30, 2.64 min (MET/CR/1278).

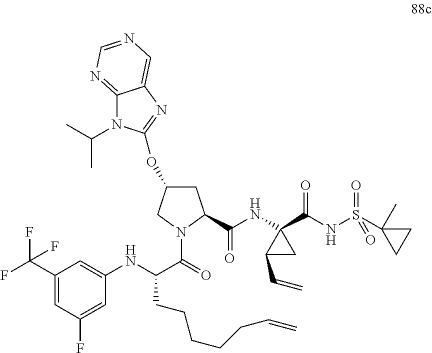

Compound 88c was prepared as described for compound 88a starting from compound 87 (207 mg, 0.328 mmol). Yield 84 mg (31%) of compound 88c as a yellow oil. ¹H NMR (500 MHz, CDCl₃) δ ppm 9.97-10.20 (m, 1H) 8.72-8.86 (m, 2H) 7.08-7.41 (m, 1H) 6.58 (br. s., 2H) 6.33 (d, J=10.68 Hz, 1H) 5.91 (d, J=1.98 Hz, 1H) 5.71-5.84 (m, 2H) 5.24 (d, J=17.09 Hz, 1H) 5.13 (dd, J=10.30, 2.82 Hz, 1H) 5.06 (d, J=9.31 Hz, 1H) 4.98 (d, J=16.94 Hz, 1H) 4.93 (d, J=10.22 Hz, 1H) 4.81 (td, J=6.79, 4.12 Hz, 1H) 4.40-4.50 (m, 1H) 4.08-4.13 (m, 3H) 2.55-2.70 (m, 2H) 2.05-2.13 (m, 1H) 1.99-2.04 (m, 3 H) 1.71-1.87 (m, 3H) 1.68 (d, J=4.88 Hz, 2H) 1.49-1.59 (m, 7H) 1.49 (d, J=2.44 Hz, 3 H) 1.42-1.47 (m, 2H) 1.29-1.43 (m, 5H). LC-MS: purity 97% (UV), m/z [M+H]⁺833.25, 2.67 min (MET/CR/1278).

Stage 5—Synthesis of compounds 501, 502 and 503:

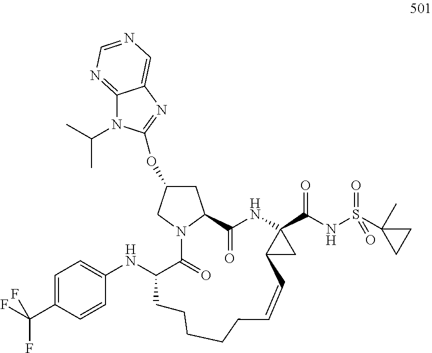

Stage 4 intermediate (compound 88a, 58 mg, 0.070 mmol, 1.0 eq.) and toluene (9 mL, previously degassed by bubbling nitrogen through the solvent for 30 min) were charged in a 25 mL round bottom flask previously flushed with nitrogen gas. Decolorizing charcoal (20 mg, ˜30 wt %) was added and the reaction mixture heated to 65° C. for 25 minutes. The charcoal was removed by filtration and the filtrate transferred to a clean 25 mL flask. Zhan catalyst (0.92 mg, 2 mol %) was added and the reaction mixture heated at 65° C. for a further 30 minutes with constant nitrogen gas bubbling through the reaction mixture (via needle). During this time the reaction mixture color turned from pale yellow to a straw color (59% conversion by LCMS-UV). Another catalyst aliquot (0.46 mg, 1 mol %) was added and the reaction mixture stirred for a further 30 minutes. As LCMS analysis showed near completion of the reaction (81% conversion by LCMS-UV) the reaction mixture was stirred for a further 30 minutes. LCMS analysis showed full consumption of the starting material. The solvent was removed under vacuum.

The residue was purified by flash column chromatography, using neat ethyl acetate as eluent. After combining the relevant fractions and solvent removal, 16 mg (29%) of the title compound was isolated as a pale brown solid. ¹H NMR (500 MHz, CDCl₃) δ ppm 10.12 (br. s., 1H) 8.71-8.94 (m, 2H) 7.15 (d, J=8.54 Hz, 2H) 7.06 (br. s., 1H) 6.49 (d, J=8.54 Hz, 2H) 5.84 (br. s., 1H) 5.68-5.80 (m, 1H) 5.00 (t, J=9.61 Hz, 1H) 4.78 (spt, J=6.84 Hz, 1H) 4.59-4.71 (m, 2H) 4.34 (d, J=11.90 Hz, 1H) 4.23-4.31 (m, 1H) 4.19 (dd, J=11.90, 3.66 Hz, 1H) 2.63-2.79 (m, 2H) 2.44 (br. s., 1H) 2.27 (q, J=8.85 Hz, 1H) 1.96-2.09 (m, 1H) 1.84-1.96 (m, 2H) 1.73-1.85 (m, 2H) 1.65 (br. s., 3H) 1.52 (s, 2H) 1.50 (s, 6H) 1.46 (d, J=10.99 Hz, 3H) 1.43 (d, J=6.87 Hz, 5H). LC-MS: purity 100% (UV), t_{R} 4.92 min m/z [M+H]⁺ 787.25 (MET/CR/1416).

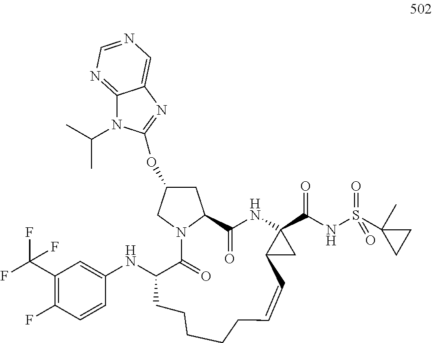

Compound 502 was prepared as described for compound 501 starting from compound 88b (98.0 mg, 0.118 mmol). Yield 14 mg (15%) of compound 502 as a pale brown solid. ¹H NMR (500 MHz, CDCl₃) δ ppm 10.14 (br. s., 1H) 8.83 (br. s., 1H) 7.15 (br. s., 1H) 6.66-6.75 (m, 2H) 6.56-6.67 (m, 1H) 5.85 (br. s., 1H) 5.67-5.78 (m, 1H) 4.99 (t, J=9.69 Hz, 1H) 4.72-4.82 (m, 1H) 4.65 (t, J=7.32 Hz, 1H) 4.39 (d, J=9.46 Hz, 1 H) 4.13-4.28 (m, 3H) 2.60-2.77 (m, 2H) 2.35-2.49 (m, 1H) 2.28 (q, J=8.70 Hz, 1H) 1.95-2.10 (m, 2H) 1.79-1.94 (m, 3H) 1.74-1.79 (m, 2H) 1.63-1.74 (m, 3H) 1.51 (d, J=6.87 Hz, 3H) 1.49 (s, 3H) 1.45 (d, J=6.87 Hz, 3H) 1.37-1.41 (m, 1H) 1.27-1.37 (m, 3 H) 0.80-0.86 (m, 2H). LC-MS: purity 97% (UV), t_{R} 4.96 min m/z [M+H]⁺ 805.25 (MET/CR/1416).

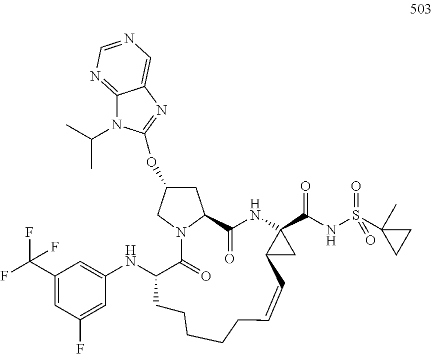

Compound 503 was prepared as described for compound 501 starting from compound 88c (84.0 mg, 0.101 mmol). Yield 26 mg (32%) of compound 503 as a pale brown solid. ¹H NMR (500 MHz, CDCl₃) δ ppm 10.16 (br. s., 1H) 8.83 (br. s., 2H) 7.21 (s, 1H) 6.55 (br. s., 2H) 6.28 (d, J=10.83 Hz, 1H) 5.86 (br. s., 1H) 5.64-5.80 (m, 1H) 4.98 (t, J=9.69 Hz, 1H) 4.75-4.83 (m, 1H) 4.74 (d, J=8.70 Hz, 1H) 4.69 (t, J=7.71 Hz, 1H) 4.16-4.30 (m, 3H) 2.71-2.81 (m, 1H) 2.61-2.71 (m, 1H) 2.34-2.50 (m, 1H) 1.98-2.12 (m, 1H) 1.84-1.97 (m, 2H) 1.69-1.85 (m, 4H) 1.55-1.58 (m, 1H) 1.53 (d, J=6.87 Hz, 3H) 1.49 (s, 3H) 1.47 (d, J=6.87 Hz, 3H) 1.38-1.45 (m, 3H) 1.26-1.38 (m, 3H) 0.78-0.87 (m, 2H). LC-MS: purity 100% (UV), t_{R} 5.09 min m/z [M+H]⁺ 805.20 (MET/CR/1416).

### 5.3 Synthesis of Precursor Compound 2-Chloro-9-isopropyl-purine

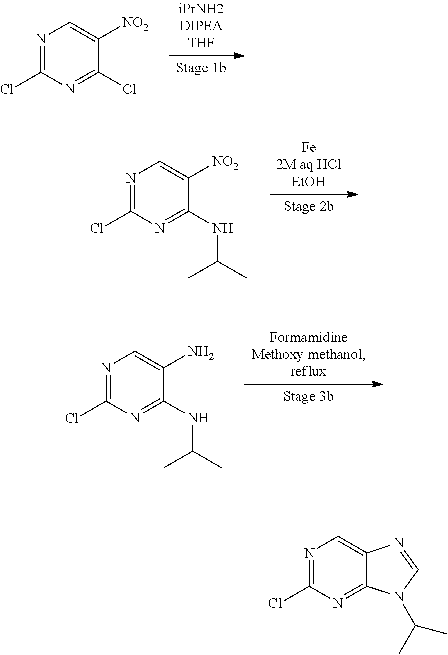

Stage 1b—2-Chloro-4-isopropylamino-5-nitro-pyrimidine: 2,4-Dichloro-5-nitro-pyrimidine (11.9 g, 61.30 mmol, 1.0 eq.) and tetrahydrofuran (180 mL) were charged into a 500 mL round bottom flask placed in ice/water bath. Diisopropylethylamine (75 mL, 0.429 mol, 7.0 eq.) was added portion wise. Isopropylamine (5.22 mL, 61.30 mmol, 1.0 eq.) was diluted with tetrahydrofuran (35 mL). The solution was added dropwise, over 15 minutes, to the reaction mixture. Stirring was continued for a further 5 minutes and checked by LCMS to show the reaction was complete. The reaction mixture was filtered and the solvent removed in vacuo. The residue was taken up in ethyl acetate (130 mL) and the organic phase washed with 10% aqueous citric acid (2×55 mL). The organic phase was dried over sodium sulfate, filtered and the solvent removed in vacuo to give a dark oil (12.9 g). The oil was purified by flash column chromatography using a heptanes: ethyl acetate gradient (from neat heptanes to 10% ethyl acetate in heptanes). After combining the relevant fractions and removing the solvent in vacuo, 8.37 g (69%) of the title compound was isolated as a yellow oil. ¹H NMR (250 MHz, CDCl₃) δ ppm 9.03 (s, 1H) 8.24 (br. s., 1H) 4.43-4.64 (m, 1H) 1.34 (d, J=6.55 Hz, 6H). LC-MS: purity 99% (UV), m/z [M+H]⁺ 216.90, 1.90 min (MET/CR/1278).

Stage 2b—2-Chloro-4-isopropylamino-5-amino-pyrimidine: 2-Chloro-4-isopropylamino-5-nitro-pyrimidine (1.0 g, 4.62 mmol, 1.0 eq.) and ethanol (15 mL) were charged into a 50 mL round bottom flask. 2 M hydrochloric acid (15 mL) was added portion wise and the reaction mixture cooled on top of in ice/water bath. Iron (1.68 g, 30.0 mmol, 6.5 eq.) was added portion wise over 5 minutes. The reaction mixture was then heated under reflux for 30 minutes by which time the reaction was complete. The iron powder was removed by filtration and the solvent removed in vacuo. The residue was diluted with dichloromethane (30 mL) and the solution washed with saturated aqueous sodium hydrogen carbonate (3×15 mL). The organic phase was dried over sodium sulfate, filtered and the solvent removed in vacuo to yield 733 mg (85% yield) of the title compound as a solid which was used in the next step without further purification. ¹H NMR (250 MHz, CDCl₃) δ ppm 7.58 (s, 1H) 4.96 (d, J=7.31 Hz, 1H) 4.21-4.44 (m, 1H) 3.02 (br. s., 2H) 1.25 (d, J=6.55 Hz, 6H). LC-MS: purity 96% (UV), t_{R} 1.22 min m/z [M+H]⁺ 186.90 (MET/CR/1278).

Stage 3b—2-Chloro-9-isopropyl-purine: 2-Chloro-4-isopropylamino-5-amino-pyrimidine (100 mg, 0.54 mmol, 1.0 eq.) was dissolved into methoxyethanol (1.5 mL). Formamidine acetate (112 mg, 1.08 mmol, 2.0 eq.) was added portion wise and the reaction mixture heated under reflux for 3 hours. The reaction mixture was left to cool down to ambient temperature and the solvent removed in vacuo. The residue was partitioned between ethyl acetate (2 mL) and water (2 mL). The aqueous phase was back extracted with ethyl acetate (2 mL). The organic phases were combined, dried over sodium sulfate, filtered and the solvent removed in vacuo. The residue was purified by flash column chromatography using a ethyl acetate/heptanes gradient to yield 76 mg (72% yield) of the title compound as a solid. ¹H NMR (250 MHz, CDCl₃) δ ppm 8.99 (s, 1H) 8.19 (s, 1H) 4.95 (spt, J=6.83 Hz, 1H) 1.66 (d, J=6.85 Hz, 6H). LC-MS: purity 99% (UV), t_{R} 1.45 min m/z [M+H]⁺ 196.90 (MET/CR/1278).

### 5.4 Synthesis of Precursor Compound 2-Chloro-9-benzyl-purine

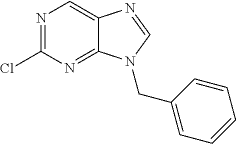

2-Chloro-9-benzyl-purine was prepared following the method described for 2-Chloro-9-isopropyl-purine, which yielded 68 mg (65%) as a beige solid. ¹H NMR (250 MHz, CDCl₃) δ ppm 9.02 (s, 1H) 8.06 (s, 1H) 7.36-7.45 (m, 3H) 7.29-7.37 (m, 2H) 5.44 (s, 2H). LC-MS: purity 99% (UV), t_{R} 1.73 min m/z [M+H]⁺ 244.95 (MET/CR/1278).

### 5.5 Synthesis of Compounds 504, 505 and 506

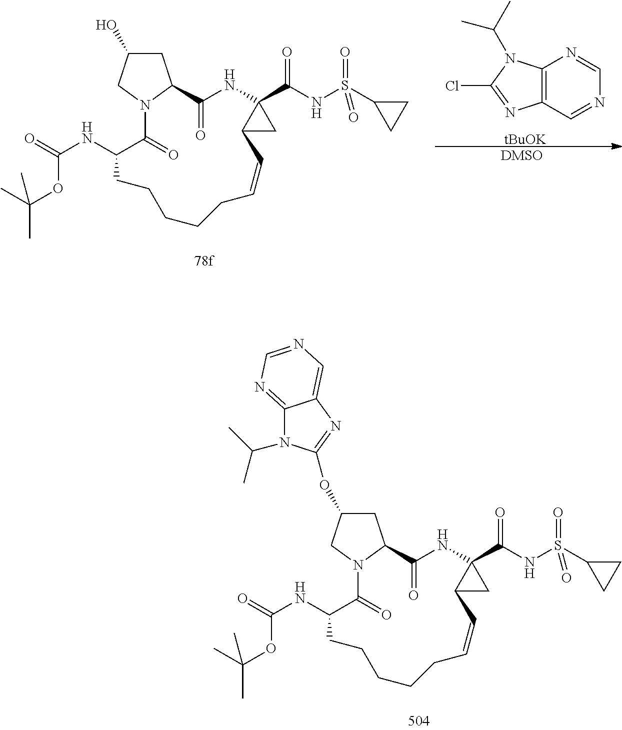

The macrocycle precursor 78f (260 mg, 0.458 mmol, 1.0 eq.), 8-Chloro-9-isopropyl-purine (90 mg, 0.458 mmol, 1 eq.) and anhydrous dimethylsulfoxide (5 mL) were charged into a 10 mL round bottom flask. Potassium tert-butoxide (334 mg, 1.83 mmol, 4.0 eq.) was added portionwise and the suspension stirred at ambient temperature for a further 2 hours. Water (20 mL) was added and the solution neutralized with 2M hydrochloric acid. The resulting solution was extracted with ethyl acetate (3×15 mL). The organic phases were combined, washed with brine (30 mL), dried over sodium sulfate, filtered and the solvent removed in vacuo. The residue was purified by flash column chromatography using a heptane/ethyl acetate/methanol gradient to yield 100 mg of a yellow oil (50% pure by LCMS-UV). The residue was further purified by preparative HPLC to give 20.6 mg (6% yield) of compound 504 as a white solid. ¹H NMR (500 MHz, CDCl₃) δ ppm 10.26 (br. s., 1H) 8.80 (d, J=5.04 Hz, 2H) 6.91-7.04 (m, 1H) 5.85 (br. s., 1H) 5.70-5.78 (m, 1H) 4.96-5.04 (m, 2H) 4.83-4.89 (m, 1H) 4.66-4.74 (m, 1H) 4.57-4.64 (m, 1H) 4.19-4.29 (m, 1H) 4.00-4.06 (m, 1H) 2.85-2.95 (m, 1H) 2.66-2.78 (m, 2H) 2.55-2.62 (m, 1H) 2.27-2.35 (m, 1H) 1.68-1.98 (m, 7H) 1.53-1.60 (m, 6H) 1.39-1.51 (m, 5H) 1.27 (s, 9H) 1.11 (br. s., 2H) 0.88-0.98 (m, 1H). LC-MS: purity 100% (UV), t_{R} 4.32 min m/z [M+H]⁺729.80 (MET/CR/1416).

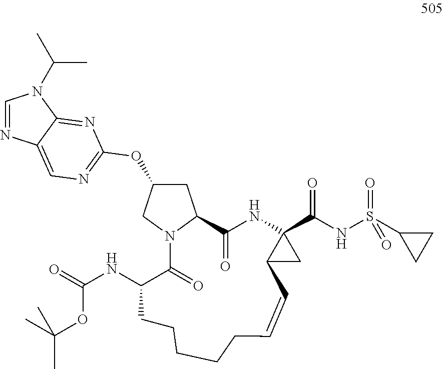

Compound 505 was prepared following the method described for compound 504, which yielded 26 mg (13%) as a white solid after preparative HPLC. ¹H NMR (500 MHz, CDCl₃) δ ppm 10.27 (s, 1H) 8.80 (s, 1H) 7.95 (s, 1H) 5.67 (br. s., 2H) 5.08-5.14 (m, 1H) 4.86-4.95 (m, 1H) 4.75-4.81 (m, 1H) 4.55-4.61 (m, 1H) 4.21-4.38 (m, 2H) 3.92-4.00 (m, 1H) 2.79-2.89 (m, 1H) 2.41-2.61 (m, 3H) 2.17-2.26 (m, 1 H) 1.74-1.88 (m, 3H) 1.52-1.65 (m, 10H) 1.28-1.46 (m, 6H) 1.19 (s, 9H) 1.02-1.11 (m, 2H) 0.81-0.88 (m, 1H). LC-MS: purity 100% (UV), t_{R} 4.41 min m/z [M+H]⁺ 729.80 (MET/CR/1416).

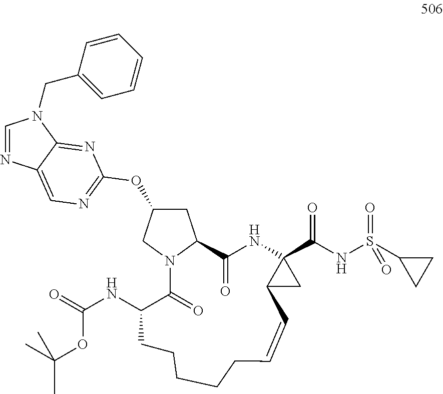

Compound 506 was prepared following the method described for compound 504, which yielded 49 mg (22%) as a white solid after preparative HPLC. ¹H NMR (500 MHz, CDCl₃) δ ppm 10.23 (s, 1H) 8.82 (s, 1H) 7.85 (s, 1H) 7.26-7.34 (m, 3 H) 7.20-7.24 (m, 2H) 7.01 (br. s., 1H) 5.61-5.69 (m, 2H) 5.24-5.34 (m, 2H) 5.03-5.07 (m, 1H) 4.88-4.94 (m, 1H) 4.57 (t, 1H) 4.31-4.36 (m, 1H) 4.18-4.24 (m, 1H) 3.88-3.95 (m, 1H) 2.81-2.88 (m, 1H) 2.40-2.57 (m, 3H) 2.16-2.25 (m, 1H) 1.79-1.89 (m, 2H) 1.38-1.54 (m, 4H) 1.22-1.35 (m, 6H) 1.19 (s, 9H) 0.99-1.10 (m, 2H) 0.82-0.90 (m, 1H). LC-MS: purity 100% (UV), t_{R} 4.54 min m/z [M+H]⁺ 777.70 (MET/CR/1416).

### Example 6

### MMQ Analogs

### 6.1 Synthesis of Precursor Compound

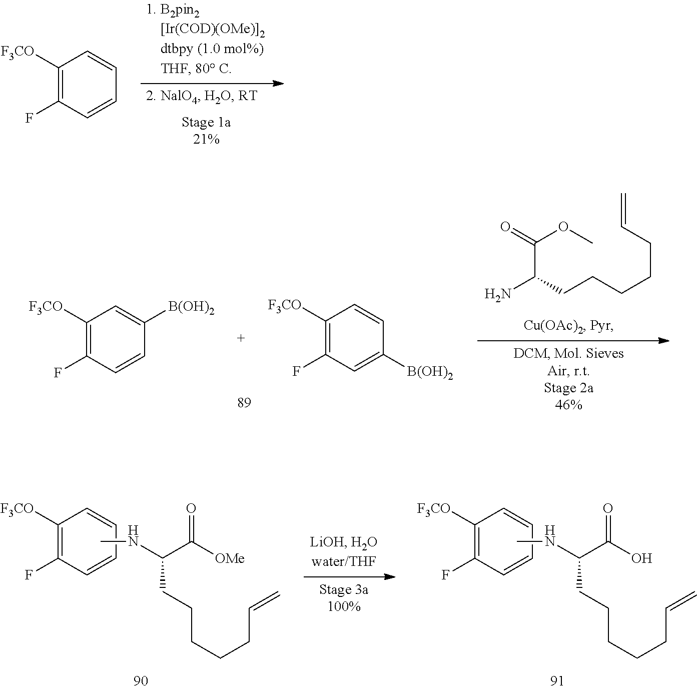

### Stage 1a: 3-(Trifluoromethoxy)-4-fluoro-phenylboronic acid (89a) and 3-fluoro-4-(trifluoromethoxy)-phenylboronic acid (89b)

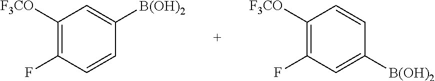

The reaction was performed in parallel in 4 sealed tubes using the following quantities. 2-Trifluoromethyl-fluorobenzene (901 mg, 5.0 mmol, 1.0 eq.), bis(pinacolato)diboron (1.09 g, 0.43 mmol, 0.86 eq.), methoxy(cyclooctadiene)Iridium(I)dimer (17 mg, 0.025 mmol, 0.5 mol %), di-tert-butylpyridine (13 mg, 0.5 mmol, 10 mol %) and tetrahydrofuran (5 mL) were charged into a sealed tube. The reaction mixture was heated to 80° C. and stirred for a further 15 hours. All four reaction mixtures were combined and water (16 mL) was added followed by sodium periodate (12.8 g, 60 mmol, 3 eq.). The reaction mixture was stirred at ambient temperature for a further 15 minutes until no more effervescence was noticed. A white suspension formed during this time. 1M Hydrochloric acid (40 mL) was added and the resulting mixture stirred at ambient temperature for 2 hours. The mixture was then extracted with ethyl acetate (3×100 mL). The organic extracts were combined, washed with water (80 mL) and brine (2×80 mL), dried over sodium sulfate, filtered and the solvent removed under vacuum. The residue was purified by flash column chromatography using a heptanes: ethyl acetate gradient (from neat heptanes to 40% ethyl acetate in heptanes). After combining the relevant fractions and solvent removal 962 mg (21%) of a mixture of two isomers was isolated as an off white solid (compound 89) which was used in the next step without further purification. LC-MS: purity 83% (UV), t_{R} 1.87 min m/z [M+H]⁺ 223.90 (MET/CR/1278).

### Stage 2a: 2-[3-(Trifluoromethoxy)-4-fluoro-phenylamino]-non-8-enoic acid methyl ester (90a) and 2-[3-fluoro-4-(trifluoromethoxy)-phenylamino]-non-8-enoic acid methyl ester (90b)

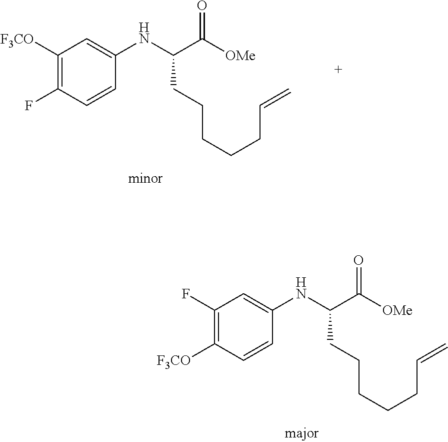

2-Amino-non-8-enoic acid methyl ester (250 mg, 1.34 mmol, 1 eq.), copper(II) acetate (268 mg, 1.47 mmol, 1.1 eq.), pyridine (0.076 mL, 2.68 mmol, 2.0 eq.) and dichloromethane (10 mL) were charged into a 25 mL round bottom flask. 4 Å Molecular sieves were added followed by stage 1a mixture of isomers 89a and 89b (600 mg, 2.68 mmol, 2.0 eq.). The reaction mixture was shaken under an air atmosphere at ambient temperature for 15 hours. The reaction mixture was acidified to pH=1 by addition of 1M hydrochloric acid. The aqueous phase was back extracted with dichloromethane (3×10 mL). The organic extracts were combined, dried over sodium sulfate, filtered and the solvent removed under vacuum. The residue was purified by flash column chromatography using a ethyl acetate: heptanes gradient (from neat heptanes to 3% ethyl acetate in heptanes). After combining the relevant fractions and solvent removal, 228 mg (46%) of the title compound 90 was isolated as a pale yellow oil. The mixture, composed of 6:4 ratio of 3-F-isomer versus 4-F-isomer was used in the next step without further purification. LC-MS: purity 93% (UV), t_{R} 2.77 min m/z [M+H]⁺ 364.35 (MET/CR/1278).

### Stage 3a: 2-[3-(Trifluoromethoxy)-4-fluoro-phenylamino]-non-8-enoic acid (91a) and 2-[3-fluoro-4-(trifluoromethoxy)-phenylamino]-non-8-enoic acid (91b)

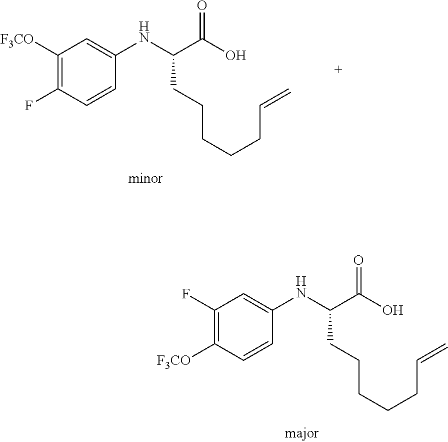

Stage 2a mixture of esters 90a and 90b (228 mg, 0.63 mmol, 1.0 eq.) and tetrahydrofuran (7 mL) were charged into a 25 mL round bottom flask. Lithium hydroxide monohydrate (79 mg, 1.88 mmol, 3.0 eq.) was dissolved in water (7 mL). The hydroxide solution was added to the reaction mixture dropwise and the resulting mixture stirred at ambient temperature for 15 hours. At this stage, LCMS analysis of the reaction mixture showed the hydrolysis to be complete. The tetrahydrofuran was removed under vacuum and the aqueous phase acidified to pH=1 with 1M hydrochloric acid. The acidic phase was extracted with dichloromethane (3×20 mL). The organic extracts were combined, dried over sodium sulfate, filtered and the solvent removed under vacuum to give 224 mg (100%) of a mixture of isomers (91a and 91b) (66:33) as a pale yellow semi solid which contained residual dichloromethane (<5% w/w). LC-MS: purity 97% (UV), t_{R} 2.51 min m/z [M+H]⁺ 350.10 (MET/CR/1278).

### 6.2 Synthesis of Macrocyclic Compounds 601 and 602

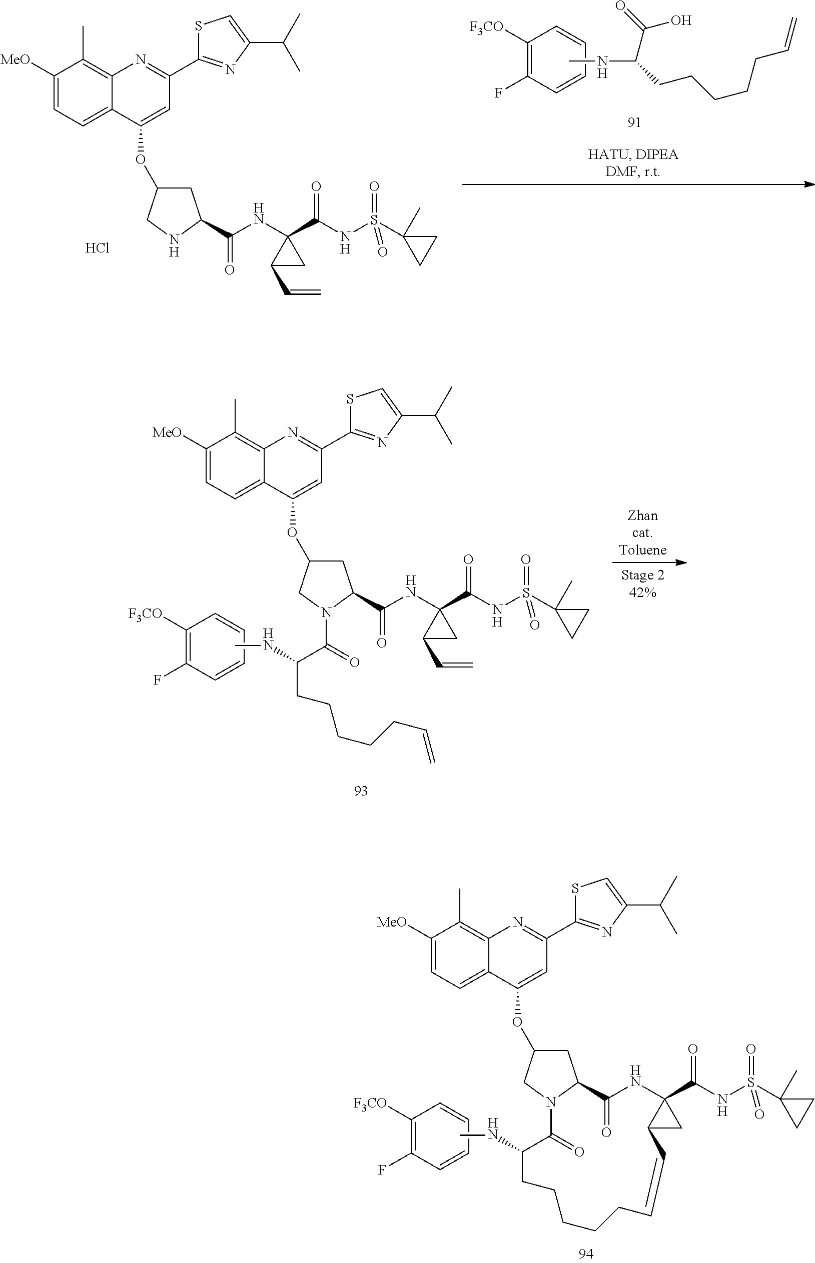

Stage 1—Synthesis of Compounds 93a and 93b:

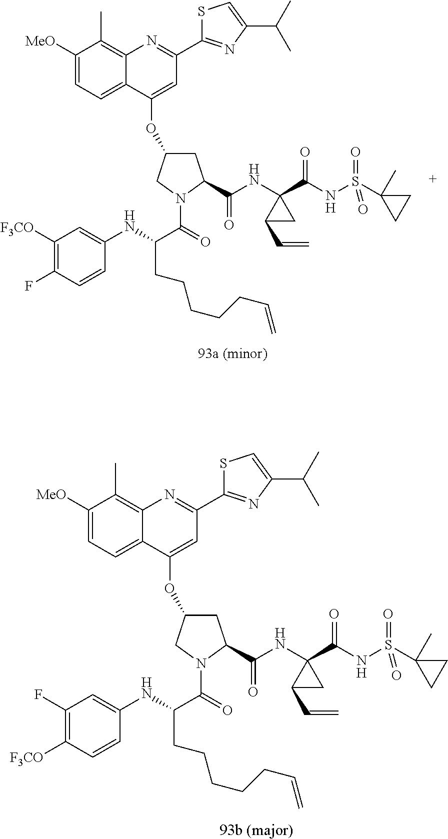

Compound 92 was prepared according to co-pending U.S. application Ser. No. 12/423,681, which is incorporated herein by reference in its entirety. The mixture of carboxylic acids isomers (91a and 91b) from Stage 3a of Example 5.1 (200 mg, 0.573 mmol, 1.1 eq.) and HATU (237 mg, 0.625 mmol, 1.2 eq.) were charged in N,N-dimethylformamide (3 mL). The reaction mixture was cooled to 0° C., and diisopropylethylamine (0.544 mL, 3.126 mmol, 6.0 eq.) and compound 92 (360 mg, 0.521 mmol, 1.0 eq.) were added in sequence. Stirring was continued for a further 2 hours. Monitoring the reaction conversion by LCMS showed full consumption of the starting material. The solvent was removed under vacuum and the residue partitioned between ethyl acetate (15 mL) and water (10 mL). The organic phase was further washed with water (4×10 mL), dried over sodium sulfate, filtered and concentrated to dryness. The residue was purified by flash column chromatography, using a heptanes: ethyl acetate gradient (from neat heptane to 40% ethyl acetate in heptanes). After combining the relevant fractions the solvent was removed under vacuum to give 220 mg (39%, off-white solid) of the title compound 93 as a mixture of isomers (93a and 93b). LC-MS: purity 100% (UV), t_{R} 5.57 min m/z [M+H]⁺ 985.36 (MET/CR/1426).

Stage 2—Synthesis of Compounds 601 and 602:

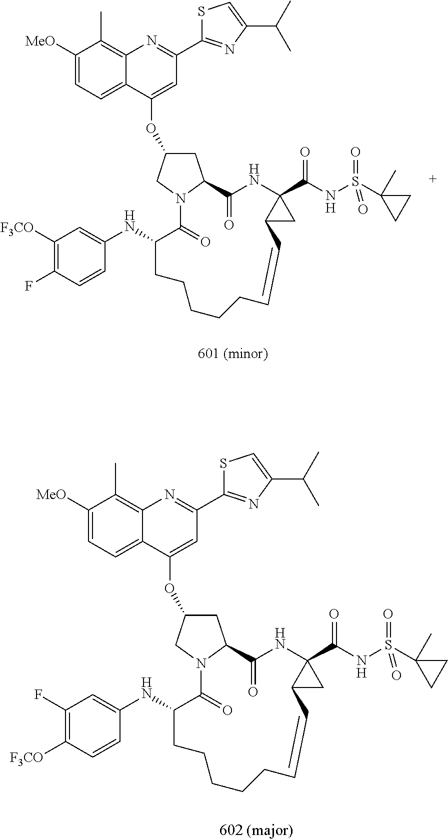

Stage 1 mixture of isomers 93a and 93b (200 mg, 0.199 mmol, 1.0 eq.) was dissolved in toluene (30 mL) and decolorizing charcoal (60 mg, — 30 wt %) was added. The slurry was heated at 65° C. for 20 minutes and the charcoal removed by filtration while still hot. The solution was transferred to a 50 mL round bottom flask and the reaction mixture heated to 65° C. Zhan catalyst (0.6 mg, 1 mol %) was added and the reaction mixture heated at 65° C. for a further 20 minutes with constant nitrogen gas bubbling through the reaction mixture (via needle). During this time the reaction mixture color turned from pale yellow to a straw color (87% conversion by LCMS-UV). Another catalyst aliquot (0.3 mg, 0.5 mol %) was added and the reaction mixture stirred for a further 30 minutes. As LCMS analysis showed some residual starting material (93% conversion by LCMS-UV) stirring was continued for another 20 minutes. LCMS-UV analysis showed full consumption of the starting material. The solvent was removed under vacuum and the residue purified by flash column chromatography, using a methanol: dichloromethane gradient (from neat dichloromethane to 0.5% methanol in dichloromethane). After combining the relevant fractions and solvent removal, 112 mg of the title compound 94 was isolated as a glassy solid. The solid was purified further by Supercritical Fluid preparative chromatography (Chiralpak Iowa (2×15 cm), 30% ethanol/0.1% diethylamine/CO₂, 100 bar, 50 mL/min, 220 nm, inj vol.: 2 mL, 9 mg/mL methanol) to give 2 fractions (compounds 601 and 602). For each fraction the diethylamine salt was released by stirring in a mixture of ethyl acetate: 1M hydrochloric acid (1:1, 1 eq. HCl). The organic phase was collected and the solvent removed under vacuum.

Fraction 1 (t_{R} 2.56 min):

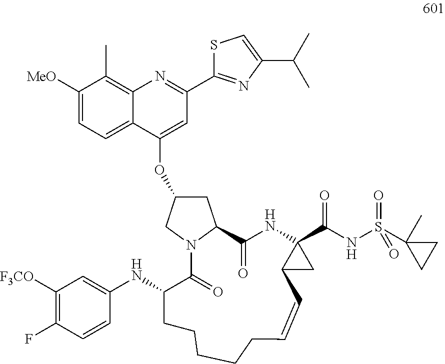

Compound 601, 24.7 mg (13%), yellow solid. ¹H NMR (500 MHz, CDCl₃) δ ppm 10.07 (s, 1H) 7.72 (d, J=9.16 Hz, 1H) 7.54 (s, 1H) 7.15 (d, J=9.16 Hz, 1H) 7.06 (s, 1H) 6.96 (br. s., 1H) 6.48-6.55 (m, 1H) 6.44 (t, J=9.38 Hz, 1H) 6.20 (dt, J=8.85, 3.13 Hz, 1H) 5.66-5.80 (m, 1H) 5.58 (br. s., 1H) 5.01 (t, J=9.54 Hz, 1H) 4.68 (t, J=7.86 Hz, 1 H) 4.18-4.24 (m, 1H) 4.08-4.18 (m, 2H) 3.98 (s, 3H) 3.22 (spt, J=6.79 Hz, 1H) 2.74 (d, J=6.26 Hz, 2H) 2.71 (s, 3H) 2.42-2.56 (m, 1H) 2.21 (q, J=8.95 Hz, 1H) 1.93-2.03 (m, 1 H) 1.92 (d, J=6.87 Hz, 1H) 1.74-1.83 (m, 3H) 1.59-1.71 (m, 2H) 1.51-1.60 (m, 1H) 1.50 (s, 3H) 1.43-1.48 (m, 3H) 1.41 (d, J=6.87 Hz, 6H) 1.25-1.35 (m, 3H) 0.83 (br. s., 2 H). LC-MS: purity 100% (UV), t_{R} 5.34 min m/z [M+H]⁺ 957.36 (MET/CR/1426)

Fraction 2 (t_{R} 3.15 min):

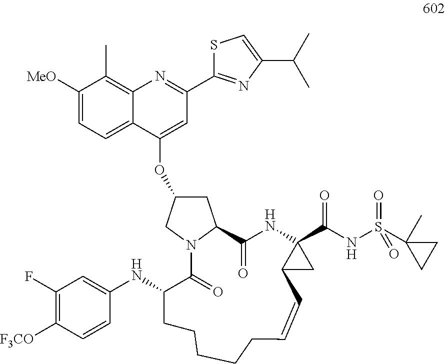

Compound 602, 54.7 mg (29%), yellow solid. ¹H NMR (500 MHz, CDCl₃) δ ppm 10.07 (s, 1H) 7.78 (d, J=9.16 Hz, 1H) 7.55 (s, 1H) 7.16 (d, J=9.16 Hz, 1H) 7.06 (s, 1H) 7.02 (s, 1H) 6.57 (t, J=8.54 Hz, 1H) 6.33 (dd, J=11.98, 2.67 Hz, 1H) 6.04 (dd, J=8.77, 2.06 Hz, 1H) 5.68-5.78 (m, 1H) 5.59 (br. s., 1H) 5.01 (t, J=9.61 Hz, 1H) 4.69 (t, J=7.93 Hz, 1H) 4.26 (d, J=11.75 Hz, 1H) 4.13-4.18 (m, 2H) 3.96 (s, 3H) 3.23 (spt, J=6.89 Hz, 1H) 2.71-2.78 (m, 2H) 2.70 (s, 3H) 2.41-2.54 (m, 1H) 2.20 (q, J=8.80 Hz, 1 H) 1.94-2.04 (m, 1H) 1.90 (dd, J=8.09, 6.10 Hz, 1H) 1.75-1.88 (m, 4H) 1.52 (br. s., 2 H) 1.50 (s, 3H) 1.44-1.49 (m, 3H) 1.41 (d, J=7.02 Hz, 6H) 1.28-1.37 (m, 3H) 0.83 (d, J=1.22 Hz, 2H). LC-MS: purity 100% (UV), t_{R} 5.34 min m/z [M+H]⁺ 957.36 (MET/CR/1426).

### Example 7

### Indole Analogs

### 7.1 Building Block Synthesis

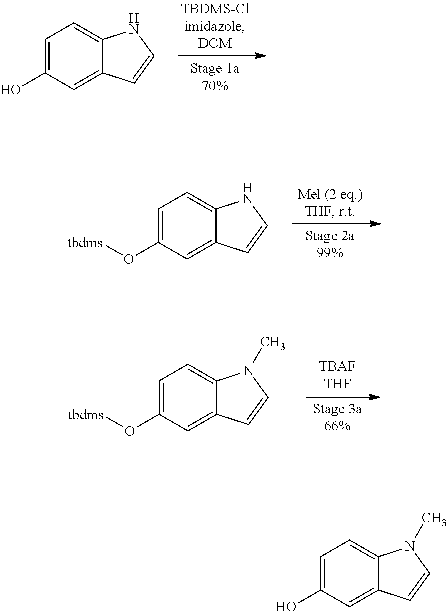

Stage 1a—synthesis of 5-{[tert-Butyl(dimethyl)silyl]oxy}-1H-indole: 1-H-indol-5-ol (1.0 g, 7.5 mmol, 1.0 eq.) and N,N-dimethylformamide (10 mL) were charged into a 50 mL round bottom flask. Imidazole (1.12 g, 16.5 mmol, 2.2 eq.) and tert-butyldimethylsilyl chloride (1.24 g, 8.3 mmol, 1.1 eq.) were dissolved in N,N-dimethylformamide (10 mL) and the resulting solution added dropwise to the reaction mixture. The reaction mixture was stirred at ambient temperature for another 15 hours. Water (40 mL) was added. The solution was extracted with ethyl acetate (2×40 mL) and the combined organic extracts washed with water (2×40 mL), dried over sodium sulfate, filtered and the solvent removed in vacuo. The residue was purified by flash column chromatography using 10% ethyl acetate in heptane. The relevant fractions were combined and the solvent removed in vacuo to give 1.3 g (70% yield) of the title compound as a pale yellow solid. ¹H NMR (500 MHz, CDCl₃) δ ppm 8.02 (br. s., 1H) 7.24 (d, J=8.70 Hz, 1H) 7.18 (t, J=2.75 Hz, 1H) 7.08 (d, J=2.29 Hz, 1H) 6.77 (dd, J=8.62, 2.37 Hz, 1H) 6.45 (t, J=2.06 Hz, 1H) 1.02 (s, 9H) 0.20 (s, 6H). LC-MS: 100% (UV), t_{R} 2.64 min m/z [M+H]⁺ 248.05 (MET/CR/1278)

Stage 2a—synthesis of 1-Methyl-5-{[tert-butyl(dimethyl)silyl]oxy}-1H-indole: 5-{[tent-Butyl(dimethyl)silyl]oxy}-1H-indole (500 mg, 2.0 mmol, 1.0 eq.) was dissolved in dry tetrahydrofuran (10 mL) and the flask was placed on top of an ice bath. Sodium hydride (60% oil suspension, 120 mg, 3.0 mmol, 1.5 eq.) was added portion wise until gas evolution has ceased. Methyl iodide (568 mg, 4.0 mmol, 2.0 eq.) was added dropwise. The mixture was stirred for another 1.5 hour and was then poured onto crushed ice. The slurry was extracted with ethyl acetate (3×20 mL) and the combined organic extracts dried over sodium sulfate, filtered and the solvent removed in vacuo to give 510 mg (95% yield) of the title compound as a pale brown oil which was used in the next step without further purification. ¹H NMR (500 MHz, CDCl₃) δ ppm 7.16 (d, J=8.70 Hz, 1H) 7.06 (d, J=2.14 Hz, 1H) 7.01 (s, 1H) 6.79 (dd, J=8.62, 2.21 Hz, 1H) 6.26-6.45 (m, 1H) 3.76 (s, 3 H) 1.01 (s, 9H) 0.20 (s, 6H). LC-MS: 87% (UV), t_{R} 2.38 min m/z [M+H]⁺ 262.05 (MET/CR/1981).

Stage 3a—synthesis of 1-Methyl-1H-indol-5-ol: 1-Methyl-5-{[tert-butyl(dimethyl)silyl]oxy}-1H-indole (510 mg, 1.95 mmol, 1.0 eq.) was dissolved in dry tetrahydrofuran (7.5 mL). Tetrabutylammonium fluoride (1.56 g, 2.34 mmol, 1.2 eq.) was added and the reaction mixture stirred at ambient temperature for 1.5 hour. The solvent was removed in vacuo. Acetonitrile (50 mL) and the precipitated solid removed by filtration. The filtrate was concentrated in vacuo and the residue purified by flash column chromatography using an ethyl acetate/heptane gradient to 190 mg (34% yield) of the title compound as a pale yellow solid which contained a small amount of bis-alkylated by-product (<5% w/w). Product used in next stage without further purification. ¹H NMR (500 MHz, CDCl₃) δ ppm 7.19 (d, J=8.70 Hz, 1H) 7.01-7.05 (m, 2H) 6.81 (dd, J=8.70, 2.44 Hz, 1H) 6.36 (d, J=2.44 Hz, 1H) 4.50 (br. s., 1H) 3.77 (s, 3H). LC-MS: 86% (UV), t_{R} 1.50 min m/z [M+H]⁺147.95 (MET/CR/1278).

### 7.2 Synthesis of Compounds 701 and 702

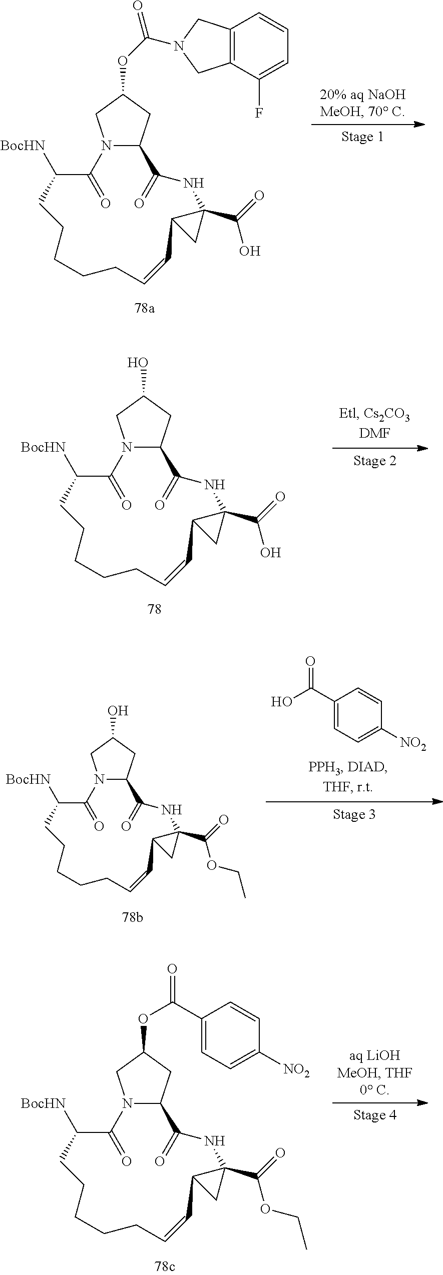

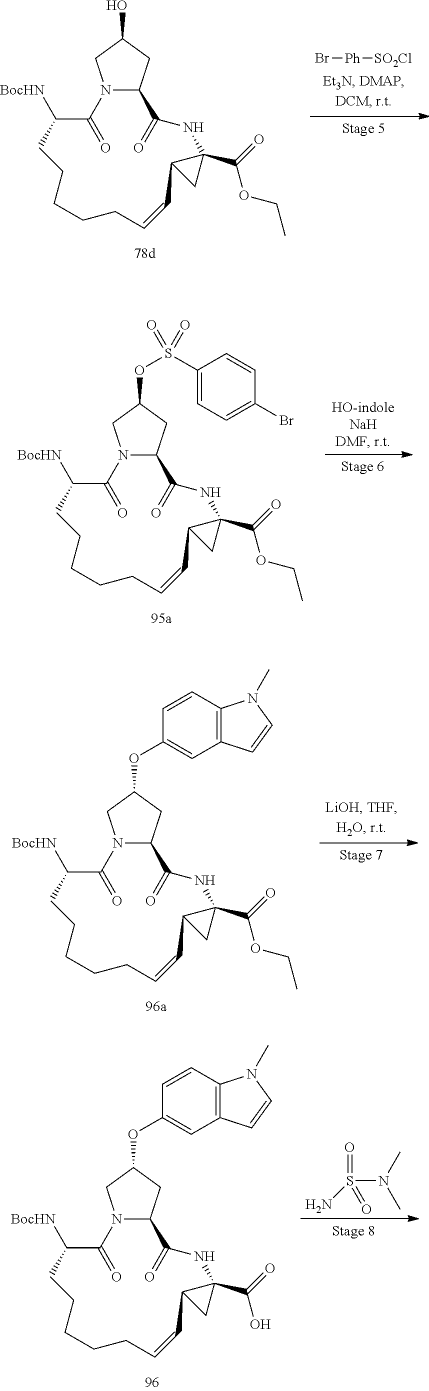

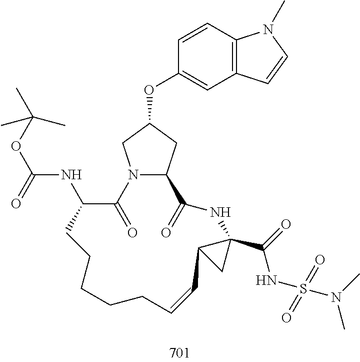

Stage 1: Compound 78a was prepared according to PCT Publication No. WO 2007/015824, which is incorporated herein by reference in its entirety. The macrocycle acid 78a (10 g, 15.9 mmol, 1 eq.) and methanol (100 mL) were charged into a 500 mL round bottom flask. 20% aqueous sodium hydroxide solution was added portionwise and the reaction mixture heated at 70° C. for 15 hours. Methanol was removed in vacuo and the residue diluted with water. The reaction mixture was cooled to 0° C. and the pH adjusted to 2-3 by dropwise addition of 2M aqueous hydrochloric acid. The aqueous phase was then extracted with ethyl acetate (3×200 mL). The organic extracts were combined, washed with brine (400 mL), dried over sodium sulfate, filtered and the solvent removed in vacuo. The residue was dissolved into methanol (80 mL) and the solution treated with decolorizing charcoal (2.0 g) under reflux for 20 min. The mixture was filtered and the solvent removed in vacuo to give 6.18 g (83% yield) of the title compound 78 as a pale brown foamy solid. ¹H NMR (500 MHz, DMSO-d₆) δ ppm 8.52 (br. s., 1H) 6.82 (d, J=7.63 Hz, 1H) 5.49 (q, 1H) 5.28 (t, J=9.77 Hz, 1H) 5.10 (d, J=3.36 Hz, 1H) 4.40 (d, J=2.29 Hz, 1H) 4.30 (t, J=7.71 Hz, 1H) 4.13-4.19 (m, 1H) 3.57-3.66 (m, 2H) 2.34-2.44 (m, 2H) 2.13 (q, J=8.85 Hz, 1 H) 1.92-1.98 (m, 2H) 1.78-1.89 (m, 1H) 1.60-1.73 (m, 1H) 1.41-1.49 (m, 2H) 1.36-1.41 (m, 3H) 1.35 (s, 9H) 1.21-1.32 (m, 4H). LC-MS: 92% (UV), t_{R} 1.76 min m/z [M+H]⁺ 466.15 (MET/CR/1278).

Stage 2: Compound 78 (6.18 g, 9.29 mmol, 1.0 eq.) and N,N-dimethylformamide (60 mL) were charged into a 250 mL round bottom flask. Ethyl iodide (2.98 g, 1.5 mL, 18.6 mmol, 2.0 eq.) and cesium carbonate (6.62 g, 18.6 mmol, 2.0 eq) were added and the reaction mixture heated at 50° C. for 1 hour. Water (270 mL) was added and the resulting milky mixture extracted with ethyl acetate (3×240 mL). The organic extracts were combined, washed with water (4×270 mL) and brine (270 mL), dried over sodium sulfate, filtered and the solvent removed in vacuo to give 6.0 g of a foamy solid. The solid was purified by flash column chromatography using a ethyl acetate/heptane gradient. After combining the relevant fractions the solvent was removed in vacuo to give 3.88 g (67% yield) of the title compound 78b as a cream solid. ¹H NMR (500 MHz, CDCl₃) δ ppm 7.22 (br. s., 1 H) 5.48-5.57 (m, 1H) 5.35 (d, J=7.78 Hz, 1H) 5.25 (t, J=9.61 Hz, 1H) 4.79 (dd, J=8.16, 5.72 Hz, 1H) 4.56 (br. s., 1H) 4.45-4.51 (m, 1H) 4.03-4.17 (m, 2H) 3.94 (d, J=11.14 Hz, 1H) 3.66 (dd, J=10.99, 4.58 Hz, 1H) 2.60 (dt, J=13.35, 5.38 Hz, 1H) 2.05-2.25 (m, 4 H) 1.81-1.93 (m, 2H) 1.53-1.67 (m, 2H) 1.44-1.51 (m, 1H) 1.42 (s, 9H) 1.28-1.40 (m, 4H) 1.26-1.29 (m, 1H) 1.20 (t, J=7.10 Hz, 4H). LC-MS: 99% (UV), t_{R} 1.91 min m/z [M+H]⁺ 494.25 (MET/CR/1278).

Stage 3: Compound 78b (1.73 g, 3.33 mmol, 1.0 eq.), 4-nitro-benzoic acid (0.568 g, 3.33 mmol, 1.0 eq.), triphenylphosphine (1.76 g, 6.66 mmol, 2.0 eq.) and dry tetrahydrofuran (86 mL) were charged into a 250 mL round bottom flask. The reaction mixture was cooled on top of an ice bath and diisopropylazodicarboxylate (DIAD, 1.38 mL, 6.66 mmol, 2.0 eq.) was added dropwise. The cooling bath was removed and stirring was continued at ambient temperature for a further 3 hours by which time t.l.c. and LCMS analyses showed full consumption of the starting material. Saturated aqueous sodium hydrogen carbonate (11 mL) was added and the reaction mixture stirred for a further 5 minutes. The reaction mixture was then extracted with dichloromethane (3×35 mL). The organic extracts were combined, dried over sodium sulfate, filtered and the solvent removed in vacuo. The residue was purified by flash column chromatography using 30% ethyl acetate in heptanes as eluent. After combining the relevant fraction and solvent removal, the title compound 78c 1.7 g (79% yield) was isolated as a brown oil. ¹H NMR (500 MHz, CDCl₃) δ ppm 8.23-8.27 (m, 2H) 8.19-8.23 (m, 2H) 7.10 (br. s., 1H) 5.65 (t, J=5.65 Hz, 1H) 5.47 (td, J=11.06, 5.04 Hz, 1H) 5.34 (d, J=8.09 Hz, 1H) 5.17 (t, J=9.69 Hz, 1H) 5.04 (d, J=8.70 Hz, 1H) 4.62 (t, J=7.48 Hz, 1H) 4.36 (dd, J=12.28, 5.87 Hz, 1H) 3.87 (d, J=12.21 Hz, 1 H) 3.65-3.73 (m, 2H) 2.98 (d, J=14.34 Hz, 1H) 2.23-2.35 (m, 2H) 2.15 (q, 1H) 1.91-2.03 (m, 3H) 1.68-1.78 (m, 2H) 1.47-1.56 (m, 2H) 1.46 (s, 9H) 1.38-1.44 (m, 1H) 1.14-1.25 (m, 3H) 1.05 (t, J=7.17 Hz, 3H). LC-MS: 92% (UV), t_{R} 2.14 min m/z [M+Na]⁺664.95 (MET/CR/1981).

Stage 4: Compound 78c (1.7 g, 2.51 mmol, 1.0 eq.), methanol (42 mL), water (42 mL) and tetrahydrofuran (85 mL) were charged into a 100 mL round bottom flask and the reaction mixture cooled on top of an ice bath for 5 minutes. 5 M aqueous lithium hydroxide (12.6 mL, 12.6 mmol, 5.0 eq.) was added dropwise to the reaction mixture and stirring was continued on top of the ice bath. The reaction extent was checked regularly by t.l.c. (UV and ninhydrin stain). After 1 hour the reaction looked complete. The reaction mixture was made neutral by addition of 1M aqueous acetic acid and then extracted with dichloromethane (3×35 mL). The organic extracts were combined, washed with saturated aqueous sodium hydrogen carbonate (85 mL), water (70 mL), and brine (70 mL). The organic phase was dried over sodium sulfate, filtered and the solvent removed in vacuo to give 1.06 g (85% yield) of the desired product 78d as creamy foam. ¹H NMR (500 MHz, CDCl₃) δ ppm 7.31 (br. s., 1H) 5.57 (td, J=9.84, 7.32 Hz, 1H) 5.29 (d, J=9.61 Hz, 1H) 5.21-5.27 (m, 1 H) 4.90 (d, J=10.68 Hz, 1H) 4.76 (d, J=8.85 Hz, 1H) 4.44-4.54 (m, 2H) 4.09-4.18 (m, 2 H) 3.90 (dd, J=11.06, 4.35 Hz, 1H) 3.75 (d, J=11.14 Hz, 1H) 2.44 (d, J=14.19 Hz, 1H) 2.05-2.25 (m, 4H) 1.77-1.88 (m, 2H) 1.64-1.75 (m, 1H) 1.54 (dd, J=9.61, 5.34 Hz, 1 H) 1.44 (s, 9H) 1.37-1.43 (m, 2H) 1.26-1.37 (m, 4H) 1.24 (t, J=7.10 Hz, 3H). LC-MS: 90% (UV), t_{R} 2.01 min, m/z [M+Na]⁺ 516.15 (MET/CR/1278).

Stage 5: Compound 78d (200 mg, 0.39 mmol, 1.0 eq.) and dry toluene (1.0 mL) were charged into a 10 mL vial. 4-Bromo-benzenesulfonyl chloride (115 mg, 0.43 mmol, 1.1 eq.) was added as a single portion and the reaction mixture cooled on top of an ice bath for 5 minutes. Potassium tert-butoxide (54 mg, 0.47 mmol, 1.2 eq.) was dissolved into tetrahydrofuran (0.4 mL) and the resulting solution added dropwise to the cold reaction mixture. The reaction mixture was stirred at ambient temperature for 15 hours. As the reaction was not complete further potassium tert-butoxide was added (0.4 eq.) and stirring was continued for another 15 hours. After this time the reaction mixture was washed with 1M aqueous sodium hydroxide (0.5 mL), 1M hydrochloric acid (0.5 mL) and water (0.5 mL). The organic phase was dried over magnesium sulfate, filtered and the solvent removed in vacuo. The residue was purified by flash column chromatography using an ethyl acetate/heptane gradient (from neat heptane to 50% ethyl acetate in heptane). After combining the relevant fractions and solvent removal, 119 mg (42% yield) of the desired product 95a was isolated as an off-white solid. ¹H NMR (500 MHz, CDCl₃) δ ppm 7.83 (d, J=8.70 Hz, 2H) 7.72 (d, J=8.70 Hz, 2H) 6.90 (s, 1H) 5.50 (td, J=10.76, 5.19 Hz, 1H) 5.28 (d, J=8.24 Hz, 1H) 5.15-5.24 (m, 2H) 4.80 (dd, J=9.00, 1.68 Hz, 1H) 4.48-4.56 (m, 1H) 4.24 (dd, J=12.13, 6.03 Hz, 1H) 4.07-4.17 (m, 1H) 3.95-4.04 (m, 1H) 3.78-3.87 (m, 1H) 2.67 (d, J=14.50 Hz, 1H) 2.21-2.35 (m, 1H) 2.04-2.18 (m, 2H) 1.91-2.04 (m, 3H) 1.69-1.75 (m, 1H) 1.60-1.69 (m, 1H) 1.48-1.53 (m, 1H) 1.44 (s, 9H) 1.34-1.40 (m, 1H) 1.28-1.33 (m, 3H) 1.23-1.25 (m, 1H) 1.20 (t, J=7.10 Hz, 3H). LC-MS: purity 98% (UV), t_{R} 2.58 min, m/z [M+Na]⁺ 734.15/736.00 (MET/CR/1278).

Stage 6: 1-Methyl-1H-indol-5-ol (31 mg, 0.21 mmol, 1.0 eq.) and N,N-dimethylformamide (1.5 mL) were charged into a 10 mL vial and the solution cooled to 5° C. on top of an ice bath. Sodium hydride (60% dispersion in oil, 8.8 mg, 0.22 mmol, 1.1 eq.) was added portion wise and the reaction mixture stirred at ambient temperature for a further 20 minutes. Compound 95a (150 mg, 0.21 mmol, 1.0 eq.) was dissolved in N,N-dimethylformamide (1.5 mL) and the solution added to the reaction mixture dropwise. The reaction mixture was stirred for 5 hours at ambient temperature. As conversion was slow, caesium carbonate (68 mg, 0.21 mmol, 1.0 eq.) was added and stirring was continued for 16 hours at 50° C. The reaction mixture was left to cool down to ambient temperature and was diluted with water (12 mL). The aqueous phase was extracted with ethyl acetate (2×12 mL). The combined organic extracts were washed with water (2×10 mL), brine (10 mL) and the solvent removed in vacuo. The residue was purified by flash column chromatography using an ethyl acetate/heptane gradient. The relevant fractions were combined and the solvent removed in vacuo to give 42 mg (51% uncorrected yield) of the title compound 96a as a white solid which contained residual starting material (<10% w/w). The solid was used in the next stage without further purification. ¹H NMR (500 MHz, CDCl₃) δ ppm 7.22 (d, J=8.85 Hz, 1H) 7.11 (s, 1H) 7.04 (d, J=3.05 Hz, 1H) 7.01 (s, 1H) 6.85 (dd, J=8.70, 2.29 Hz, 1H) 6.41 (d, J=3.05 Hz, 1H) 5.48-5.57 (m, 2H) 5.44-5.48 (m, 1H) 5.22-5.29 (m, 1H) 4.99-5.09 (m, 1H) 4.84-4.92 (m, 1H) 4.56-4.65 (m, 1H) 4.06-4.20 (m, 3H) 3.94-4.03 (m, 1 H) 3.83-3.94 (m, 1H) 3.77 (s, 3H) 2.82-2.91 (m, 1H) 2.08-2.32 (m, 5H) 1.83-1.98 (m, 2H) 1.72-1.82 (m, 1H) 1.60-1.69 (m, 1H) 1.52-1.60 (m, 1H) 1.39-1.47 (m, 9H) 1.14-1.34 (m, 5H). LC-MS: purity 89% (UV), t_{R} 2.47 min, m/z [M+Na]⁺ 645.30 (MET/CR/1278).

Stage 7: Compound 96a (42 mg, 0.07 mmol, 1.0 eq.), tetrahydrofuran (0.4 mL), methanol (0.2 mL) and water (0.2 mL) were charged into a 10 mL vial. Lithium hydroxide monohydrate (17 mg, 0.40 mmol, 6.0 eq.) was added portion wise and the reaction mixture heated at 40° C. for 4 hours. Stirring was continued at ambient temperature for 16 hours. The solvent was removed in vacuo and the residue diluted with water (5 mL). 0.5 M hydrochloric acid (2 mL) was added and the solution extracted with dichloromethane (3×20 mL). The combined organic extracts were dried over sodium sulfate, filtered and the solvent removed in vacuo to give 39 mg (97% yield) the title compound 96 as a pale yellow solid which was used in the next stage without further purification. ¹H NMR (500 MHz, CDCl₃) δ ppm 7.05-7.10 (m, 1H) 7.12 (d, J=8.85 Hz, 1H) 7.01 (br. s., 1H) 6.95 (d, J=2.75 Hz, 1 H) 6.74 (dd, J=8.70, 2.29 Hz, 1H) 6.32 (d, J=2.75 Hz, 1H) 5.43-5.59 (m, 1H) 5.32-5.43 (m, 1H) 5.10-5.19 (m, 1H) 4.84-4.96 (m, 1H) 4.56-4.70 (m, 1H) 4.36-4.47 (m, 1H) 3.91-4.06 (m, 1H) 3.69-3.76 (m, 1H) 3.68 (s, 3H) 2.43-2.59 (m, 1H) 2.23-2.40 (m, 1 H) 1.88-2.19 (m, 3H) 1.60-1.85 (m, 2H) 1.38-1.58 (m, 3H) 1.35 (s, 9H) 1.06-1.32 (m, 6H). LC-MS: purity 92% (UV), t_{R} 2.21 min, m/z [M+Na]⁺ 617.40 (MET/CR/1278).

Stage 8: Compound 96 (39 mg, 0.07 mmol, 1.0 eq.) and dichloroethane (0.7 mL) were charged into a 7 mL vial. 1,1-Carbonyldiimidazole (13.0 mg, 0.08 mmol, 1.2 eq.) was added as a single portion and the suspension heated at 50° C. for 1.5 hours. N,N-dimethylsulfamide (12 mg, 0.10 mmol, 1.5 eq.) was added as a single portion followed by dropwise addition of 1,8-diazabicylco[5.4.0]undec-7-ene (18 mg, 0.11 mmol, 1.5 eq.). Stirring was continued at 50° C. for 5 hours and then at ambient temperature for 15 hours. The solvent was removed in vacuo and the residue purified by flash column chromatography using ethyl acetate/heptane/formic acid (40:60:1) as eluent. The relevant fractions were combined and the solvent removed in vacuo to give 10 mg (22% yield) of the title compound 701 as an off-white foamy solid. ¹H NMR (500 MHz, CDCl₃) δ ppm 9.94-10.06 (m, 1H) 7.19-7.26 (m, 1H) 7.09-7.19 (m, 1H) 7.04-7.07 (m, 1H) 7.00-7.04 (m, 1H) 6.80-6.89 (m, 1H) 6.35-6.44 (m, 1H) 5.66-5.79 (m, 1H) 5.26-5.35 (m, 1H) 5.06-5.17 (m, 1H) 4.96-5.05 (m, 1H) 4.58-4.65 (m, 1H) 4.32-4.43 (m, 1H) 4.24-4.32 (m, 1H) 3.81-3.98 (m, 1 H) 3.77 (s, 3H) 2.88 (s, 6H) 2.55-2.63 (m, 1H) 2.44-2.51 (m, 1H) 2.22-2.32 (m, 1H) 1.76-1.96 (m, 4H) 1.67-1.75 (m, 1H) 1.53-1.67 (m, 2H) 1.46-1.53 (m, 1H) 1.42 (s, 9 H) 1.33-1.39 (m, 3H) 1.28-1.33 (m, 1H). LC-MS: purity 92% (UV), t_{R} 4.89 min, m/z [M+Na]⁺ 723.40 (MET/CR/1416).

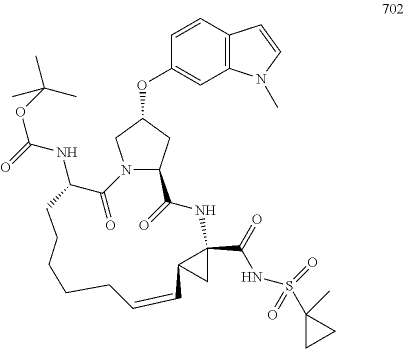

Compound 702 was prepared following the methods described herein. The yield was 85 mg (88%) as a beige solid after flash column chromatography. ¹H NMR (500 MHz, CDCl₃) δ ppm 10.11 (br. s., 1H) 7.51 (d, J=8.54 Hz, 1H) 6.99 (d, J=2.90 Hz, 1H) 6.84 (d, J=15.11 Hz, 1H) 6.74 (dd, J=8.55, 1.98 Hz, 1H) 6.42 (d, J=2.75 Hz, 1H) 5.68-5.76 (m, 1H) 5.27 (d, J=8.09 Hz, 1H) 5.11 (br. s., 1H) 5.01 (t, J=9.54 Hz, 1H) 4.59 (t, J=7.63 Hz, 1H) 4.42 (t, J=8.39 Hz, 1H) 4.30 (d, J=10.99 Hz, 1H) 3.89 (d, J=8.24 Hz, 1H) 3.75 (s, 3H) 2.44-2.63 (m, 3H) 2.26-2.35 (m, 1H) 1.87-1.98 (m, 2H) 1.76-1.87 (m, 2 H) 1.56-1.61 (m, 1H) 1.52 (d, J=10.68 Hz, 1H) 1.49 (s, 3H) 1.42-1.48 (m, 2H) 1.40 (s, 9 H) 1.35-1.38 (m, 3H) 1.31 (d, J=8.24 Hz, 3H) 0.79-0.86 (m, 2H). LC-MS: purity 99% (UV), t_{R} 5.01 min m/z [M+Na]⁺ 734.45 (MET/CR/1416).

### Example 8

### Aryltetrazole Analogs

### 8.1 Building Block Synthesis

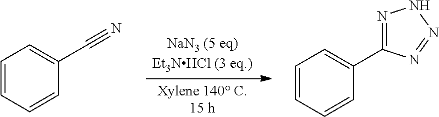

Preparation of 5-phenyl-tetrazole: Benzonitrile (410 mg, 3.97 mmol, 1.0 eq.) and xylene (6 mL) were charged in a 20 mL pressure tube. Sodium azide (1.30 g, 19.9 mmol, 5.0 eq.) and triethylamine hydrochloride (1.67 g, 11.9 mmol, 3.0 eq.) were added and the suspension heated under reflux for 16 hours. The reaction mixture was left to cool down to room temperature and partitioned between ethyl acetate (36 mL) and 10% aqueous citric acid solution (24 mL). The organic phase was washed with water (2×12 mL) and brine (2×12 mL), dried over sodium sulfate, filtered and the solvent removed in vacuo to give 516 mg (89% yield) of the title compound as a beige solid. ¹H NMR (500 MHz, DMSO-d₆) δ ppm 8.04 (dd, J=7.48, 1.98 Hz, 2H) 7.57-7.65 (m, 3H). LC-MS: purity 100% (UV), t_{R} 1.29 min, m/z [M+H]⁺ 146.90 (MET/CR/1278).

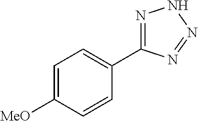

Preparation of 5-(4-methoxy-phenyl)-tetrazole: 4-Methoxy-benzonitrile (550 mg, 4.01 mmol, 1.0 eq.) and xylene (6 mL) were charged in a 20 mL pressure tube. Sodium azide (1.30 g, 19.9 mmol, 5.0 eq.) and triethylamine hydrochloride (1.67 g, 11.9 mmol, 3.0 eq.) were added and the suspension heated under reflux for 16 hours. The reaction mixture was left to cool down to room temperature and partitioned between ethyl acetate (36 mL) and 10% aqueous citric acid solution (24 mL). The organic phase was washed with water (2×12 mL) and brine (2×12 mL), dried over sodium sulfate, filtered and the solvent removed in vacuo to give 531 mg (75% yield) of the title compound as a beige solid. ¹H NMR (500 MHz, DMSO-d₆) δ ppm 7.98 (d, J=9.00 Hz, 2H) 7.16 (d, J=8.85 Hz, 2H) 3.84 (s, 3H). LC-MS: purity 100% (UV), t_{R} 1.40 min, m/z [M+H]⁺ 176.95 (MET/CR/1278).

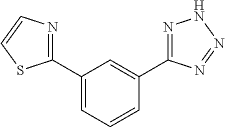

Preparation of 5-(3-thiazol-2-yl-phenyl)-tetrazole: 3-(Thiazol-2-yl)-benzonitrile (400 mg, 1.93 mmol, 1.0 eq.) and xylene (6 mL) were charged in a 20 mL pressure tube. Sodium azide (0.635 g, 9.7 mmol, 5.0 eq.) and triethylamine hydrochloride (0.815 g, 5.8 mmol, 3.0 eq.) were added and the suspension heated under reflux for 16 hours. The reaction mixture was left to cool down to room temperature and partitioned between ethyl acetate (36 mL) and 10% aqueous citric acid solution (24 mL). The organic phase was washed with water (2×12 mL) and brine (2×12 mL), dried over sodium sulfate, filtered and the solvent removed in vacuo to give 340 mg (76% yield) of the title compound as a beige solid. ¹H NMR (500 MHz, DMSO-d₆) δ ppm 8.65 (s, 1H) 8.15 (dt, J=7.78, 1.83 Hz, 2H) 8.02 (d, J=3.20 Hz, 1H) 7.90 (d, J=3.20 Hz, 1H) 7.75 (t, J=7.78 Hz, 1H). LC-MS: purity 99% (UV), t_{R} 1.56 min, m/z [M+H]⁺ 229.90 (MET/CR/1278).

### 8.2 Synthesis of Compounds 801-805

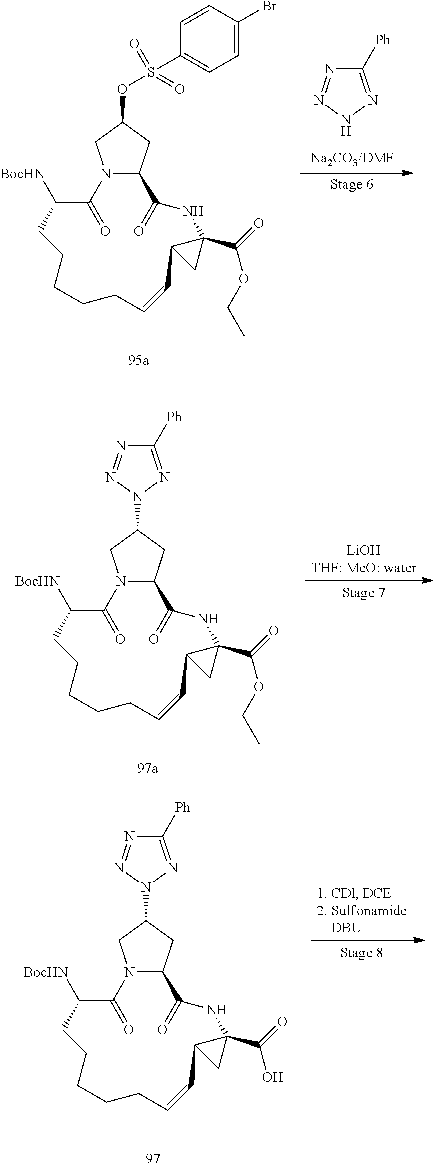

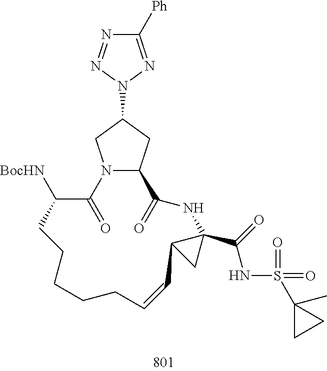

Preparation of Stages 1 to 5 intermediates has been described above in section 7.2.

Stage 6: Compound 95a (120 mg, 0.16 mmol, 1.0 eq.), 5-Phenyl-tetrazole (71 mg, 0.48 mmol, 3.0 eq) and N,N-dimethylformamide (6 mL) were charged into a 12 mL vial. Sodium carbonate (104 mg, 0.96 mmol, 6.0 eq.) was added portion wise and the reaction mixture heated at 60° C. for 15 hours. The reaction mixture was diluted with water (24 mL) and extracted with ethyl acetate (3×18 mL). The combined organic extracts were washed with water (3×12 mL) and brine (12 mL), dried over magnesium sulfate, filtered and the solvent removed in vacuo to give 98 mg (98% yield) of the title compound 97a as a yellow oil. ¹H NMR (500 MHz, CDCl₃) δ ppm 8.05-8.14 (m, 2H) 7.40-7.50 (m, 3H) 7.27 (br. s., 1H) 5.61-5.74 (m, 1H) 5.46-5.57 (m, 1H) 5.35 (d, J=8.09 Hz, 1H) 5.18-5.28 (m, 1H) 4.95-5.07 (m, 1H) 4.44-4.53 (m, 1H) 4.35-4.44 (m, 1H) 4.19-4.25 (m, 1H) 4.15 (d, J=7.17 Hz, 1H) 4.06-4.12 (m, 1H) 3.05-3.20 (m, 1H) 2.72-2.83 (m, 1H) 2.05-2.26 (m, 3H) 1.79-1.94 (m, 2H) 1.58-1.72 (m, 1H) 1.49-1.58 (m, 1H) 1.33-1.48 (m, 6H) 1.30 (s, 9H) 1.24-1.28 (m, 3H). LC-MS: purity 92% (UV), t_{R} 2.35 min, m/z [M+Na]⁺644.30 (MET/CR/1278).

Stage 7: Compound 97a (109 mg, 0.16 mmol, 1.0 eq.), tetrahydrofuran (0.8 mL), methanol (0.4 mL) and water (0.4 mL) were charged into a 10 mL vial. Lithium hydroxide monohydrate (40 mg, 0.95 mmol, 6.0 eq.) was added portion wise and the reaction mixture stirred at ambient temperature for 16 hours. The solvent was removed in vacuo and the residue diluted with ethyl acetate (5 mL). Water (5 mL) was added and the pH of the aqueous phase adjusted to 2-3 with 1M hydrochloric acid. The organic phase was collected and the aqueous further extracted with ethyl acetate (2×5 mL). The combined organic extracts were dried over magnesium sulfate, filtered and the solvent removed in vacuo to give 88 mg (94% yield) the title compound 97 as an off-white solid which was used in the next stage without further purification. ¹H NMR (500 MHz, CDCl₃) δ ppm 8.08 (br. s., 2H) 7.48-7.57 (m, 1H) 7.44 (br. s., 3H) 5.65-5.85 (m, 1H) 5.56 (br. s., 1H) 5.33-5.43 (m, 1H) 5.16-5.28 (m, 1H) 4.92-5.09 (m, 1H) 4.44 (br. s., 2H) 4.16-4.29 (m, 1H) 2.97-3.20 (m, 1H) 2.75-2.93 (m, 1H) 2.21 (br. s., 2H) 2.08-2.15 (m, 1H) 1.83 (br. s., 2H) 1.59 (br. s., 2H) 1.32-1.46 (m, 5H) 1.26-1.31 (m, 9H) 1.23-1.25 (m, 2H). LC-MS: purity 95% (UV), t_{R} 2.12 min, m/z [M+Na]⁺ 612.25 (MET/CR/1278).

Stage 8: Compound 97 (88 mg, 0.15 mmol, 1.0 eq.) and dichloroethane (1.6 mL) were charged into a 7 mL vial. 1,1-Carbonyldiimidazole (37 mg, 0.22 mmol, 1.5 eq.) was added as a single portion and the suspension heated at 50° C. for 1.5 hours. Methylcyclopropylsulfonamide (30 mg, 0.22 mmol, 1.5 eq.) was added as a single portion followed by dropwise addition of 1,8-diazabicylco[5.4.0]undec-7-ene (51 mg, 0.33 mmol, 1.5 eq.). Stirring was continued at 50° C. for 5 hours. The solvent was removed in vacuo and the residue purified by flash column chromatography using ethyl acetate/heptane/formic acid (40:60:1) as eluent. The relevant fractions were combined and the solvent removed in vacuo to give 71 mg (67% yield) of the title compound 801 as an off-white foamy solid. ¹H NMR (500 MHz, CDCl₃) δ ppm 10.18 (s, 1H) 8.12 (d, J=3.51 Hz, 1H) 7.44-7.50 (m, 3H) 7.12 (s, 1H) 5.73 (d, J=8.24 Hz, 2H) 5.04 (d, J=9.31 Hz, 2H) 4.89 (t, J=7.55 Hz, 1H) 4.69 (d, J=11.29 Hz, 1H) 4.19-4.28 (m, 2H) 3.05-3.17 (m, 1H) 2.85-2.97 (m, 1H) 2.46-2.64 (m, 1H) 2.24 (q, J=8.49 Hz, 1H) 1.86-1.97 (m, 2H) 1.75-1.84 (m, 2H) 1.61-1.72 (m, 1 H) 1.53 (br. s., 2H) 1.50 (s, 3H) 1.48 (br. s., 1H) 1.42 (d, J=0.92 Hz, 2H) 1.30-1.39 (m, 3 H) 1.30 (br. s., 1H) 1.19 (s, 9H) 0.84 (d, J=1.53 Hz, 2H). LC-MS: purity 100% (UV), t_{R} 4.92 min, m/z [M+Na]⁺ 733.40 (MET/CR/1416).

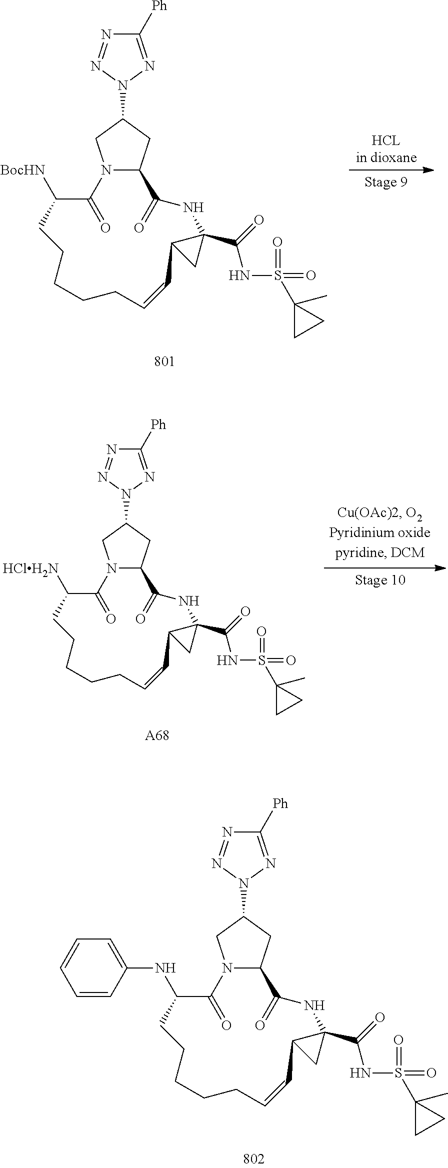

Compound 801 (64.0 mg, 0.086 mmol, 1 eq.) was dissolved into dioxane (0.45 mL). 4M HCl in dioxane (0.21 mL, 0.860 mmol, 10 eq.) was added dropwise and the reaction mixture stirred at ambient temperature for 15 hours by which time LCMS analysis of an aliquot showed the reaction to be complete. The solvent was removed in vacuo to give 52 mg (99% yield) of compound A68 as a pale yellow solid. The solid was used in the next step without further purification. LC-MS: purity 100% (UV), t_{R} 1.68 min, m/z [M+H]⁺ 611.25 (MET/CR/1981).

Compound A68 (52 mg, 0.080 mmol, 1.0 eq.) was partitioned between ethyl acetate (1 mL) and aqueous sodium hydrogen carbonate (1 mL). The two phase mixture was stirred for 10 minutes and then the organic phase collected, dried over sodium sulfate, filtered, and the solvent removed in vacuo. The residue was diluted with dichloromethane (2.6 mL, previously degassed with air for 30 minutes) and the solution transferred to a 10 mL vial. Phenylboronic acid (31 mg, 0.24 mmol, 3.0 eq.), pyridine (0.065 mL, 0.81 mmol, 10 eq.), pyridinium N-oxide (119 mg, 1.21 mmol) and copper (II) acetate (31 mg, 0.16 mmol, 2.0 eq.) were added and the reaction mixture stirred under an air atmosphere for 15 hours. Ethyl acetate (10 mL) was added leading to the precipitation of a blue-green solid which was removed by filtration. The filtrate was concentrated in vacuo and the residue purified by flash column chromatography using ethyl acetate/heptane/formic acid (50:50:1) as eluent. The relevant fractions were combined and the solvent removed in vacuo to give 22 mg (39% yield) of compound 802 as a beige solid. ¹H NMR (500 MHz, CDCl₃) δ ppm 10.23 (s, 1H) 8.01-8.18 (m, 2H) 7.69-7.85 (m, 1H) 7.40-7.52 (m, 3H) 6.91-7.02 (m, 2H) 6.51-6.64 (m, 1 H) 6.30-6.44 (m, 1H) 5.64-5.85 (m, 2H) 4.93-5.12 (m, 1H) 4.52-4.72 (m, 6H) 4.29-4.47 (m, 2H) 4.11-4.20 (m, 1H) 2.84-3.00 (m, 1H) 2.59-2.72 (m, 1H) 2.46-2.59 (m, 1 H) 2.07-2.16 (m, 1H) 1.74-2.00 (m, 6H) 1.51 (s, 3H) 1.44-1.55 (m, 2H) 1.36-1.43 (m, 2H) 0.80-0.87 (m, 2H). LC-MS: purity 100% (UV), t_{R} 4.95 min m/z [M+H]⁺ 687.21 (MET/CR/1416).

Compounds 803-805 were prepared using the methods described for preparing compounds 801 or 802 above.

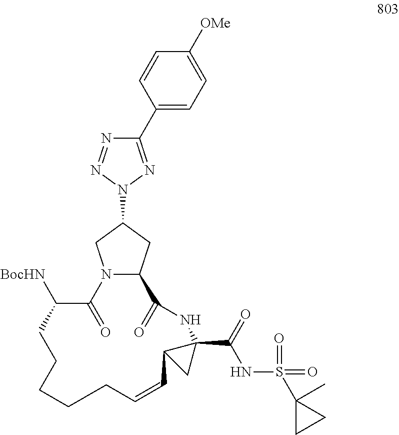

Yielded 103 mg (74% yield) as a white foam. ¹H NMR (500 MHz, CDCl₃) δ ppm 10.27 (s, 1H) 8.04 (d, J=8.54 Hz, 2H) 7.53 (s, 1H) 6.97 (d, J=8.85 Hz, 2H) 5.64-5.75 (m, 2H) 5.17 (d, J=7.78 Hz, 1H) 5.01 (t, J=9.46 Hz, 1H) 4.88 (t, J=7.55 Hz, 1H) 4.66 (d, J=11.29 Hz, 1H) 4.26 (d, J=4.12 Hz, 2H) 3.86 (s, 3H) 3.03-3.19 (m, 1H) 2.76-2.91 (m, 1H) 2.45-2.62 (m, 1H) 2.17-2.32 (m, 1H) 1.85-1.94 (m, 2H) 1.71-1.82 (m, 2H) 1.49 (s, 3H) 1.41-1.47 (m, 2H) 1.38-1.41 (m, 3H) 1.32-1.37 (m, 2H) 1.29 (d, J=6.26 Hz, 2H) 1.21 (s, 9H) 0.81-0.83 (m, 2H). LC-MS: purity 98% (UV), t_{R} 4.90 min m/z [M+Na]⁺ 763.25 (MET/CR/1416).

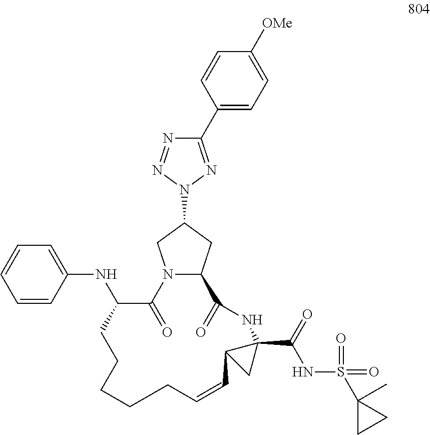

Yielded 3.2 mg (4% yield) as a white foam. ¹H NMR (500 MHz, CDCl₃) δ ppm 8.02 (d, J=8.55 Hz, 2H) 7.40-7.53 (m, 1H) 6.98-7.08 (m, 2H) 6.95 (d, J=8.70 Hz, 2 H) 6.38-6.64 (m, 1H) 6.11-6.38 (m, 1H) 5.64-5.81 (m, 2H) 5.04 (t, J=9.46 Hz, 1H) 4.57-4.69 (m, 1H) 4.37-4.45 (m, 1H) 4.30-4.37 (m, 1H) 3.86-3.90 (m, 1H) 3.86 (s, 3 H) 2.88-2.97 (m, 1H) 2.65-2.76 (m, 1H) 2.47-2.60 (m, 1H) 2.10-2.17 (m, 1H) 1.87-1.99 (m, 3H) 1.75-1.87 (m, 3H) 1.53-1.62 (m, 5H) 1.51 (s, 3H) 1.44 (br. s., 3H) 1.30 (br. s., 2H) 0.82-0.87 (m, 2H). LC-MS: purity 88% (UV), t_{R} 4.89 min m/z [M+H]⁺ 717.25 (MET/CR/1416).

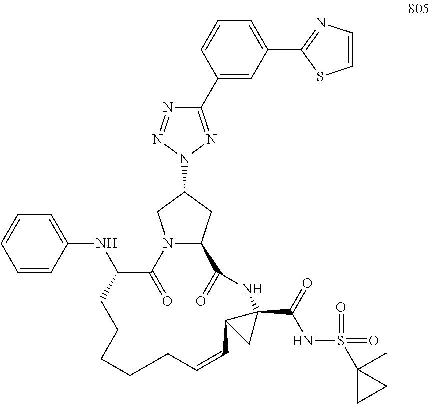

Yielded 26 mg (14% yield) as a white solid. ¹H NMR (500 MHz, CDCl₃) δ ppm 10.22 (br. s., 1H) 8.76 (br. s., 1H) 8.23 (d, J=7.78 Hz, 1H) 8.07 (d, J=7.78 Hz, 1H) 7.94 (d, J=2.90 Hz, 1H) 7.58 (t, J=7.78 Hz, 1H) 7.42 (d, J=3.05 Hz, 1H) 7.35 (br. s., 1H) 5.77 (d, J=5.34 Hz, 1H) 5.69-5.76 (m, 1H) 5.02-5.10 (m, 2H) 4.99 (d, J=8.09 Hz, 1H) 4.71 (d, J=10.68 Hz, 1H) 4.25 (dd, J=11.22, 5.11 Hz, 1H) 4.19 (t, J=9.84 Hz, 1H) 3.06-3.13 (m, 1H) 2.95 (dt, J=13.89, 6.79 Hz, 1H) 2.54-2.65 (m, 1H) 2.22-2.29 (m, 1H) 1.95 (t, J=6.71 Hz, 1H) 1.84-1.92 (m, 1H) 1.77-1.84 (m, 2H) 1.56-1.62 (m, 2H) 1.55 (br. s., 1H) 1.52 (s, 3H) 1.40-1.51 (m, 3H) 1.28-1.40 (m, 3H) 1.14 (s, 9H) 0.85 (br. s., 2H). LC-MS: purity 100% (UV), t_{R} 5.01 min m/z [M+Na]⁺ 794.40 (MET/CR/1416).

### Example 9

### Quinazoline Analogs

### 9.1 Building Block Synthesis

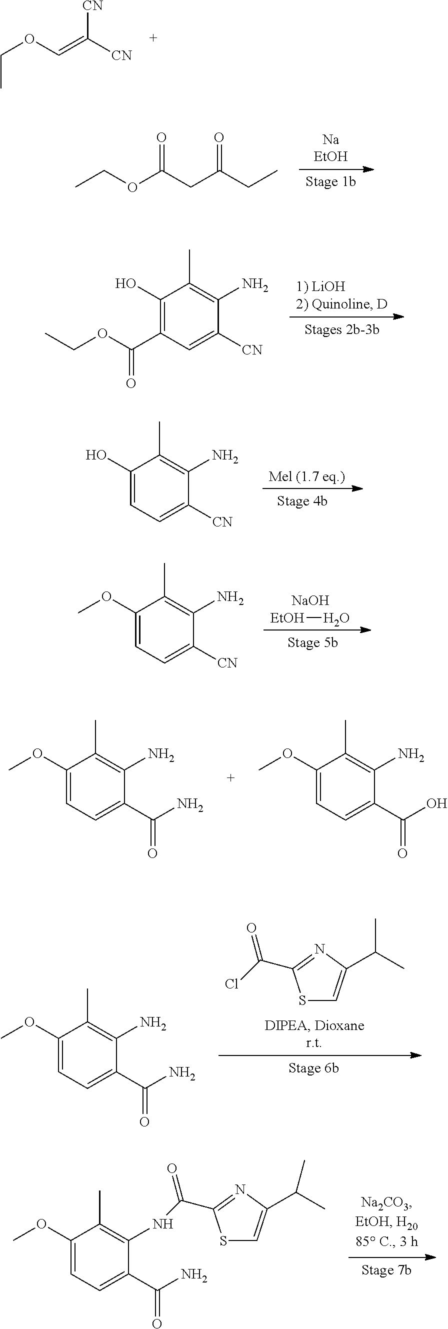

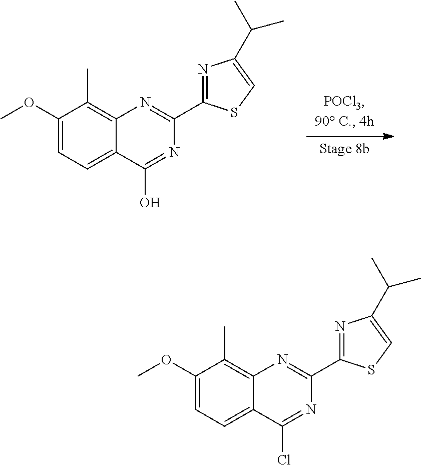

Stage 1b—Ethyl 2-Hydroxy-3-methyl-4-amino-5-cyano-benzoate: Ethanol was charged into a 1 L 3 neck flask and the solvent was warmed to 50° C. Sodium (3.27 g, 142.2 mmol, 2.05 eq.) was added in small portion over 30 minutes. Heating was continued until all the sodium lumps were dissolved. The reaction mixture was then cooled to 0° C., and ethylpropionyl acetate (10 g, 69.4 mmol, 1.0 eq) was added dropwise. The reaction mixture was stirred at ambient temperature for 1 hour then ethoxymethylene malononitrile (8.47 g, 69.4 mmol, 1.0 eq.) was added portion wise. The reaction mixture was heated under reflux for a further 2 hours, then left to cool down to ambient temperature over 15 hours with stirring. The solution was neutralised to pH=7 by slow addition of 1.5 M hydrochloric acid. The solvent was then removed in vacuo. The residue was triturated with water (50 mL) and the obtained solid collected by filtration. The crude solid was washed with 5% ethyl acetate in heptane, filtered and dried under high vacuum to yield 11.9 g (78% yield) of the title compound as a yellow orange powder. ¹H NMR (500 MHz, CDCl₃) δ ppm 11.69 (s, 1H) 7.94 (s, 1H) 4.75 (br. s., 2H) 4.38 (q, J=7.17 Hz, 2H) 2.07 (s, 3H) 1.41 (t, J=7.10 Hz, 3 H). LC-MS: purity 89% (UV), t_{R} 2.09 min m/z [M+H]⁺ 220.95 (MET/CR/1278)

Stage 2b—2-Hydroxy-3-methyl-4-amino-5-cyano-benzoic: Lithium hydroxide monohydrate (4.54 g, 108.2 mmol, 2.0 eq.) was dissolved into water (75 mL). The solution was diluted with ethanol (75 mL) and ethyl 2-hydroxy-3-methyl-4-amino-5-cyano-benzoate (11.91 g, 54.07 mmol, 1.0 eq.) was added portion wise. The reaction mixture was heated at 80° C. for 4 hours then let to cool down to ambient temperature. The solvent was removed in vacuo and the residue was portioned between water (80 mL) and ethyl acetate/heptane (1:1, 80 mL). The aqueous layer was collected, acidified to pH=5 with 1.5 M hydrochloric acid and extracted with ethyl acetate (3×100 mL). The combined organic extracts were washed with brine (100 mL), dried over magnesium sulfate, filtered and the solvent removed in vacuo to give 9.56 g (92% yield) of the title compound as a yellow solid which was used in the next stage without further purification. ¹H NMR (500 MHz, MeOD) δ ppm 7.88 (br. s., 1H) 2.04 (s, 3H). LC-MS: purity 88% (UV), t_{R} 1.46 min m/z [M+H]⁺193.00 (MET/CR/1278).

Stage 3b—2-methyl-3-Hydroxy-5-cyano-aniline: 2-Hydroxy-3-methyl-4-amino-5-cyano-benzoic (9.56 g, 49.74 mmol, 1.0 eq.) and quinoline (25 mL) were charged into a 50 mL round bottom flask. The suspension was heated at 170° C. for 2 hours until gas evolution ceased. The solution was cooled to ambient temperature and 1M aqueous sodium hydroxide solution was added. The aqueous phase was washed with hexane (3×250 mL) to remove the quinoline. The aqueous phase was then acidified to pH=5 with 1.5 M hydrochloric acid leading to the formation of a solid which was collected by filtration. The aqueous phase was further extracted with ethyl acetate (2×200 mL). The solid was dissolved into the combined organic extracts and the resulting solution washed with brine (200 mL), dried over sodium sulfate, filtered and the solvent removed in vacuo to give 6.41 g (86% yield) of the title compound as a dark yellow solid. ¹H NMR (500 MHz, MeOD) 8 ppm 7.07 (d, J=8.54 Hz, 1H) 6.21 (d, J=8.54 Hz, 1H) 2.00 (s, 3H). LC-MS: purity 998% (UV), t_{R} 1.25 min m/z [M+H]⁺ 148.90 (MET/CR/1278).

Stage 4b—2-Methyl-3-methoxy-5-cyano-aniline: 2-methyl-3-Hydroxy-5-cyano-aniline (6.4 g, 43.2 mmol, 1.0 eq.), potassium carbonate (5.9 g, 43.2 mmol, 1.0 eq.) and N,N-dimethylformamide (100 mL) were charged into a 250 mL flask. Methyl iodide (3.2 g, 51.8 mmol, 1.2 eq.) was added and the reaction mixture stirred at ambient temperature for 15 hours. The reaction mixture was diluted with water (400 mL) and extracted with 30:1 ethyl acetate/heptane (3×150 mL). The combined organic layers were washed with water (2×200 mL), brine (200 mL), dried over sodium sulfate, filtered and the solvent removed in vacuo to give 5.34 g (76%) of the title compound as a brown sticky solid which was used in the next step without further purification. ¹H NMR (500 MHz, MeOD) δ ppm 7.25 (d, J=8.85 Hz, 1H) 6.42 (d, J=8.85 Hz, 1H) 3.83 (s, 3H) 2.01 (s, 3H). LC-MS: purity 96% (UV), t_{R} 1.57 min m/z [M+H]⁺ 162.85 (MET/CR/1981).

Stage 5b—2-Amino-3-methyl-4-methoxy-benzamide: 2-Methyl-3-methoxy-5-cyano-aniline (1.0 g, 6.15 mmol, 1.0 eq.) was dissolved in ethanol (8 mL). 2M sodium hydroxide solution (8 mL, 15.4 mmol, 2.5 eq.) was added and the reaction mixture stirred under reflux for 8 hours. The reaction mixture was left to cool down for 1 hour and the precipitated solid collected by filtration. The creamy solid was further dried under high vac for 4 hours. Crop 1: 629 mg (57% yield). The filtrate was heated overnight under reflux for a further 15 hours. The reaction mixture was left to cool down to ambient temperature leading to the precipitation of more creamy solid which was collected by filtration and dried under high vacuum for 4 hours. Crop 2: 112 mg (10% yield). Overall 741 mg (67% yield) of the title compound was isolated which was used in the next step without further purification. ¹H NMR (500 MHz, DMSO-d₆) δ ppm 7.62 (br. s., 1H) 7.47 (d, J=8.85 Hz, 1H) 6.91 (br. s., 1 H) 6.51 (s, 2H) 6.24 (d, J=8.85 Hz, 1H) 3.75 (s, 3H) 1.89 (s, 3H). LC-MS: purity 97% (UV), t_{R} 1.24 min m/z [M+H]⁺ 180.95 (MET/CR/1278).

Stage 6b—2-[(3-isopropyl-thiazol-2-yl)-carbonylamino]-3-methyl-4-methoxy-benzamide: Oxalyl chloride (1.1 mL, 12.6 mmol, 3.6 eq) was added dropwise, at ambient temperature, to a solution of 4-isopropyl-thiazole-2-carboxylic acid (742 mg, 4.2 mmol, 1.2 eq) in toluene (7.5 mL). Stirring was continued at ambient temperature until the bubbling stopped. The reaction mixture was then heated under reflux for a further 1 hour. LCMS analysis of an aliquot quenched with methanol revealed full conversion of the acid to the acid chloride. The reaction mixture was left to cool down to ambient temperature and the solvent removed under vacuum. The residue was diluted with dry dioxane (7.5 mL). Diisopropylethylamine (1.2 mL, 7.0 mmol, 2.0 eq.) was added dropwise followed by 2-Amino-3-methyl-4-methoxy-benzamide (629 mg, 3.49 mmol, 1.0 eq.). The reaction mixture was stirred at ambient temperature for 15 hours. LCMS analysis showed full conversion of the starting material to product. The solvent was removed under vacuum and the residue dissolved with ethyl acetate (15 mL). The organic layer was washed with saturated aqueous sodium hydrogen carbonate (9 mL), water (9 mL), and brine (9 mL), dried over sodium sulphate, filtered and the solvent removed in vacuo to give 642 mg (55% yield, crop 1) of the title compound as a pale brown solid.

The aqueous phase was further extracted with ethyl acetate (3×15 mL). The combined organic phases were washed with brine (20 mL), dried over sodium sulfate, filtered and the solvent removed in vacuo to give 260 mg (22% yield, Crop 2) of the title compound as a white solid. Both crop 1 and crop 2 showed similar analyses. Overall, 902 mg (78% yield) of the title compound was isolated. ¹H NMR (500 MHz, DMSO-d₆) δ ppm 11.26 (s, 1H) 7.93 (s, 1H) 7.69 (s, 1H) 7.56 (d, J=8.70 Hz, 1H) 7.45 (s, 1H) 6.96 (d, J=8.70 Hz, 1H) 3.87 (s, 3H) 3.13 (spt, J=6.87 Hz, 1H) 2.00 (s, 3H) 1.31 (d, J=6.87 Hz, 6H). LC-MS: purity 100% (UV), t_{R} 1.91 min m/z [M+H]⁺ 334.05 (MET/CR/1278).

Stage 7b—2-(4-isopropylthiazol-2-yl)-4-hydroxy-7-methoxy-8-methyl-quinazoline: Stage 5b intermediate (903 mg, 2.71 mmol, 1.0 eq.), ethanol (10 mL) and water (10 mL) were charged into a 50 mL round bottom flask. Sodium carbonate (717 mg, 6.77 mmol, 2.5 eq.) was added and the reaction mixture stirred under reflux for 3 hours. The reaction mixture was left to cool down over 16 hours to let the product precipitate out slowly. The solid was collected by filtration, rinsing the cake with a little cold ethanol. Further drying under high vacuum yielded 666 mg (78% yield) of the title compound as a white powder. ¹H NMR (500 MHz, CDCl₃) δ ppm 10.09 (br. s., 1H) 8.21 (d, J=8.70 Hz, 1H) 7.16 (s, 1H) 7.11 (d, J=8.85 Hz, 1H) 3.99 (s, 3H) 3.15 (spt, J=6.79 Hz, 1H) 2.54 (s, 3H) 1.36 (d, J=7.02 Hz, 6H). LC-MS: purity 100% (UV), t_{R} 2.41 min m/z [M+H]⁺ 316.00 (MET/CR/1278).

Stage 8b—2-(4-isopropylthiazol-2-yl)-4-chloro-7-methoxy-8-methyl-quinazoline: 2-(4-isopropylthiazol-2-yl)-4-hydroxy-7-methoxy-8-methyl-quinazoline (100 mg, 0.35 mmol, 1.0 eq.) was charged into a 10 mL vial. Phosphorous oxychloride (1.5 mL) was added and the reaction mixture stirred at 90° C. for 2 hours. Monitoring the reaction mixture by ¹H NMR showed full consumption of the starting material. The reaction mixture was left to cool down to ambient temperature and the solvent removed under vacuum. The residue was diluted with ethyl acetate (20 mL) and the reaction mixture cooled to 0° C. 2M aqueous sodium hydroxide solution was added portion wise until the pH of the aqueous phase was 14. The aqueous phase was further extracted with ethyl acetate (3×50 mL). The combined organic layers were washed with water (50 mL) and brine (50 mL). The organic layer was dried over sodium sulphate, filtered and the solvent removed under vacuum to give 108 mg (93%) of the title compound as a pale brown solid. ¹H NMR (500 MHz, DMSO-d₆) δ ppm 8.21 (d, J=9.16 Hz, 1H) 7.75 (d, J=9.31 Hz, 1H) 7.61 (s, 1H) 4.06 (s, 3H) 3.18 (spt, J=6.87 Hz, 1H) 2.57 (s, 3H) 1.33 (d, J=6.87 Hz, 6H). LC-MS: purity 100% (UV), t_{R} 1.69 min m/z [M+H]⁺ 333.95 (MET/CR/1278).

### 9.2 Synthesis of Compound 901

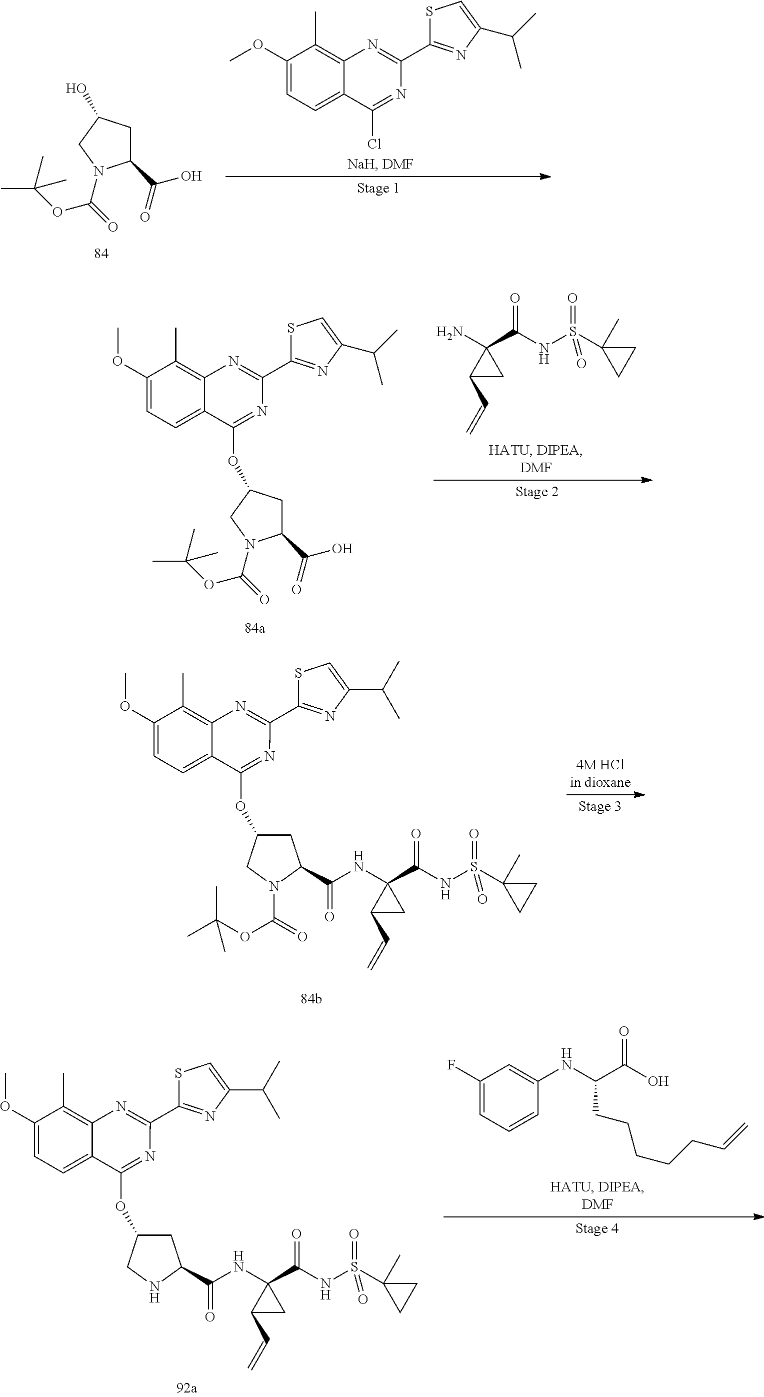

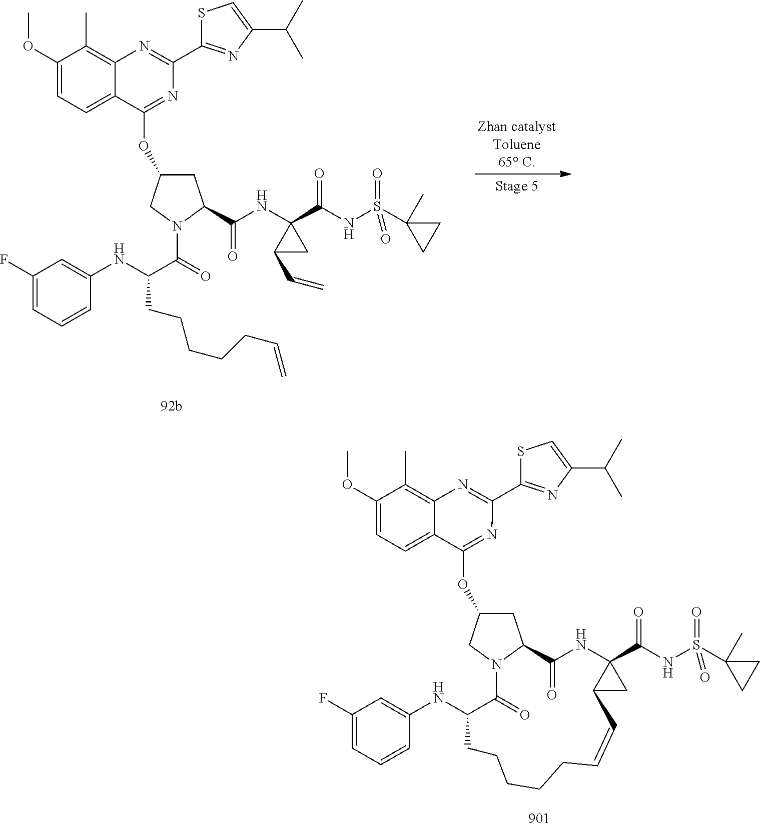

Stage 1—(2S,4R)-1-(tert-butoxycarbonylamino)-4-[2-(3′-isopropyl-thiazol-2-yl)-7-methoxy-8-methyl-quinazoline-4-oxy]-proline: (2S,4R)-1-(tert-Butoxycarbonylamino)-4-hydroxy-proline (84) (35 mg, 0.157 mmol, 1.0 eq.) and N,N-dimethylformamide (1.0 mL) were charged into a 10 mL vial and the reaction mixture cooled down to 0° C. Sodium hydride (60% dispersion in oil, 12 mg, 0.317 mmol, 2.0 eq.) was added portion wise and the reaction mixture stirred for a further 10 minutes. 2-(4-isopropylthiazol-2-yl)-4-chloro-7-methoxy-8-methyl-quinazoline (50 mg, 0.157 mmol, 1.0 eq.) was added portionwise. Stirring was continued at ambient temperature for 2 hours. The reaction mixture was quenched with methanol (1 mL) and stirred for 30 min. Water (4 mL) was added and the aqueous phase acidified to pH=3 with 1M hydrochloric acid leading to the formation of a solid which was collected by filtration. Further drying under high vacuum gave 57 mg (72% yield) of the title compound 84a as a grey solid which contained some 2-(4-isopropylthiazol-2-yl)-4-methoxy-7-methoxy-8-methyl-quinazoline (˜8% w/w). Product used in next stage without further purification. ¹H NMR (500 MHz, CDCl₃) δ ppm 7.97 (d, J=9.16 Hz, 2H) 7.21 (t, 1 H) 7.07 (d, J=11.44 Hz, 1H) 6.05 (d, 1H) 4.45-4.73 (m, 1H) 4.33 (t, 0H) 3.97 (d, J=2.44 Hz, 3H) 3.83-3.96 (m, 2H) 3.23-3.43 (m, 1H) 2.67-2.80 (m, 1H) 2.64 (s, 3H) 2.53-2.62 (m, 1H) 1.76-1.83 (m, 1H) 1.44 (s, 9H) 1.38 (t, J=4.96 Hz, 6H). LC-MS: purity 80% (UV), t_{R} 2.17 min m/z [M+H]⁺ 529.30 (MET/CR/1278).

Stage 2: (2S,4R)-1-(tert-Butoxycarbonylamino)-4-[2-(3′-isopropyl-thiazol-2-yl)-7-methoxy-8-methyl-quinazoline-4-oxy]-proline (84a) (528 mg, 0.208 mmol, 1.0 eq.) and N,N-dimethylformamide (2 mL) were charged into a 25 mL round bottom flask under nitrogen. HATU (103 mg, 0.270 mmol, 1.3 eq.) and diisopropylethylamine (0.217 mL, 1.248 mmol, 6.0 eq.) were added at 0° C. and the reaction mixture stirred at ambient temperature for a further 30 minutes. (1R,2S)-1-Amino-2-vinyl-cyclopropane-1-carbonyl-(1′-methyl)cyclopropane-sulfonamide hydrochloride salt (244 mg, 0.208 mmol, 1.0 eq.), previously dissolved in N,N-dimethylformamide (2 mL) was added dropwise over 15 minutes at 0° C. and stirring was continued for 2 hours ambient temperature. Monitoring the reaction conversion by LCMS showed complete consumption of the starting material. The solvent was removed under vacuum and the residue partitioned between water (12 mL) and ethyl acetate (12 mL). The phases were separated and the aqueous phase further extracted with ethyl acetate (2×10 mL). The combined organic extracts were washed with water (2×10 mL) and brine (10 mL), dried over sodium sulfate, filtered and the solvent removed in vacuo to give 150 mg (95% crude yield) of the title compound 84b which was used in the next step without further purification. ¹H NMR (500 MHz, CDCl₃) ppm 9.98 (br. s., 1H) 7.91 (d, J=9.00 Hz, 1H) 7.29-7.48 (m, 1H) 7.16 (d, J=9.16 Hz, 1H) 7.02-7.10 (m, 1H) 5.95 (br. s., 1H) 5.70-5.85 (m, 1H) 5.17-5.34 (m, 1H) 5.05 (d, J=10.53 Hz, 1H) 4.39 (t, J=8.01 Hz, 1H) 3.92 (s, 3H) 3.86-3.91 (m, 1H) 3.76 (d, J=12.66 Hz, 1H) 3.24 (dt, J=13.73, 6.87 Hz, 1H) 2.70-2.77 (m, 3H) 2.38-2.54 (m, 1H) 2.14-2.28 (m, 1H) 1.82-2.02 (m, 1H) 1.70-1.82 (m, 1H) 1.58-1.70 (m, 1H) 1.47 (s, 3H) 1.38-1.45 (m, 9H) 1.34-1.36 (m, 2H) 1.31-1.33 (m, 6H) 0.73-0.87 (m, 2H). LC-MS: purity 100% (UV), t_{R} 2.36 min m/z [M+H]⁺ 754.90 (MET/CR/1981).

Stage 3: Compound 84b (160 mg, 0.211 mmol, 1.0 eq.) and dioxane (1.5 mL) were charged into a 10 mL vial. 4M HCl in dioxane (1.5 mL) was added dropwise and the reaction mixture stirred at ambient temperature for 1 hour. LCMS analysis showed full consumption of the starting material. The solvent was removed under vacuum and the residue dried further under high vacuum for 4 hours to give 137 mg (99% yield) of the title compound 92a as a yellow solid. LC-MS: purity 100% (UV), t_{R} 1.48 min m/z [M+H]⁺ 655.30 (MET/CR/1981).

Stage 4: (2S)-2-(3-fluoro-phenylamino)-non-8-enoic acid (55 mg, 0.209 mmol, 1.0 eq.) and N,N-dimethylformamide (2.1 mL) were charged into a 10 mL vial under nitrogen. HATU (103 mg, 0.271 mmol, 1.3 eq.) and diisopropylethylamine (0.22 mL, 1.26 mmol, 6.0 eq.) were added at 0° C. and the reaction mixture stirred at ambient temperature for a further 30 minutes. Compound 92a (HCl salt, 137 mg, 0.21 mmol, 1.0 eq.) was added as a single portion and stirring was continued at ambient temperature for a further 2 hours. Monitoring the reaction conversion by LCMS showed full consumption of the starting material. The solvent was removed under vacuum and the residue partitioned between ethyl acetate (5 mL) and water (5 mL). The organic phase was further washed with water (5 mL×4), dried over sodium sulfate, filtered and concentrated to dryness. The residue was purified by flash column chromatography, using a dichloromethane/ethyl acetate gradient. After combining the relevant fractions the solvent was removed under vacuum to give 72 mg (38%) of the title compound 92b as a yellow solid. ¹H NMR (500 MHz, CDCl₃) δ ppm 10.17 (br. s., 1H) 7.83 (d, J=8.85 Hz, 1H) 7.39 (br. s., 1H) 7.15 (d, J=9.00 Hz, 1H) 6.36 (br. s., 2H) 6.13-6.25 (m, 2H) 5.71-5.87 (m, 2H) 5.22 (d, J=17.09 Hz, 1H) 5.10 (d, J=10.53 Hz, 1H) 4.98 (d, J=17.09 Hz, 1H) 4.92 (d, J=9.92 Hz, 1H) 4.57-4.84 (m, 1H) 4.50 (t, J=8.24 Hz, 1H) 4.04-4.20 (m, 3H) 3.99 (s, 3H) 3.18-3.34 (m, 1H) 2.64 (s, 3H) 2.42-2.59 (m, 2H) 2.13 (q, J=8.80 Hz, 1H) 1.94-2.03 (m, 2H) 1.60-1.87 (m, 4H) 1.52-1.60 (m, 1H) 1.51 (s, 3H) 1.43-1.50 (m, 2H) 1.39 (d, J=5.95 Hz, 6H) 1.28-1.37 (m, 3H) 1.21-1.27 (m, 3H) 0.91 (d, J=6.41 Hz, 1H) 0.87 (br. s., 1H) 0.80-0.86 (m, 1H). LC-MS: purity 100% (UV), t_{R} 2.54 min m/z [M+H]⁺ 901.95 (MET/CR/1981).

Stage 5: Compound 92b (70 mg, 0.076 mmol, 1.0 eq.) and toluene (7 mL) were charged into a 25 mL flask. Decolorizing charcoal (21 mg) was added and the suspension heated at 65° C. for 20 min. Charcoal was removed by filtration and the cake rinsed with further toluene (3.5 mL). The filtrate was transferred to a 25 mL round bottom flask and degassed by bubbling nitrogen through the solvent for 15 min (it is important to keep the reaction mixture under a protective nitrogen atmosphere). Zhan catalyst (1.0 mg, 2 mol %) was added and the reaction mixture heated at 65° C. for a further 20 min with constant nitrogen gas bubbling through the reaction mixture (via needle). During this time the reaction mixture color turned from pale yellow to a straw color (90% conversion by LCMS-UV). Another catalyst aliquot (1.0 mg, 2 mol %) was added and the reaction mixture stirred for a further 20 min. LCMS-UV analysis showed full consumption of the starting material. The solvent was removed under vacuum.

The residue was purified by flash column chromatography, using a methanol/dichloromethane gradient (from neat dichloromethane to 0.5% methanol in dichloromethane). After combining the relevant fractions and solvent removal, 16 mg (24%) of the title compound 901 was isolated as a beige solid. ¹H NMR (500 MHz, CDCl₃) d ppm 10.23 (br. s., 1H) 7.79 (d, J=9.00 Hz, 1H) 7.36 (br. s., 1H) 7.15 (s, 1H) 7.08 (d, J=9.00 Hz, 1H) 6.82-6.92 (m, 1H) 6.22-6.33 (m, 3H) 6.14 (d, J=11.14 Hz, 1H) 5.67-5.81 (m, 1H) 5.02 (t, J=9.61 Hz, 1H) 4.61 (t, J=7.48 Hz, 1H) 4.33-4.45 (m, 1H) 4.25-4.33 (m, 1 H) 4.11-4.24 (m, 2H) 3.93 (s, 3H) 3.25-3.42 (m, 1H) 2.70-2.79 (m, 1H) 2.65 (s, 3H) 2.55-2.62 (m, 1H) 2.45-2.55 (m, 1H) 2.13-2.22 (m, 1H) 1.91-2.07 (m, 2H) 1.90 (dd, J=7.78, 6.10 Hz, 1H) 1.77-1.87 (m, 4H) 1.56 (br. s., 1H) 1.51 (s, 3H) 1.49 (br. s., 2H) 1.43 (d, J=7.02 Hz, 6H) 1.28-1.39 (m, 2H) 1.19-1.24 (m, 1H) 0.84 (dd, J=3.51, 2.44 Hz, 2H). LC-MS: purity 98% (UV), t_{R} 4.99 min m/z [M+H]⁺ 874.40 (MET/CR/1426).

### Example 10

### Sodium Salts

10.1 Synthesis of compound 1001

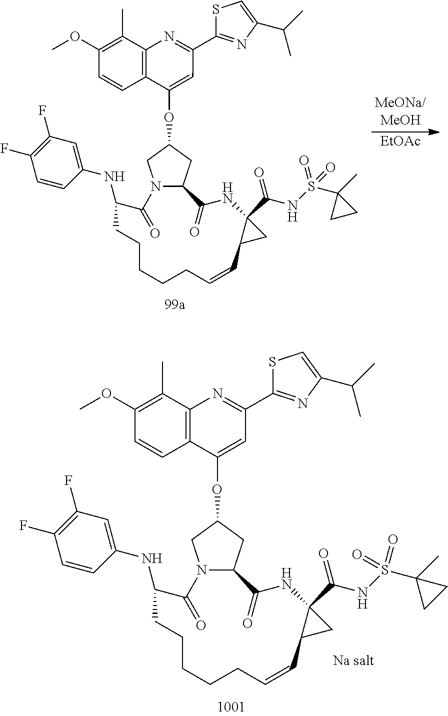

To the solution of compound 99a (prepared according to co-pending U.S. application Ser. No. 12/423,681) (1 eq.) in EtOAc was added MeONa (1 eq.)/MeOH solution (30%) slowly at 0° C., the mixture was stirred at 0° C. for 1 h. Then the solvent was vacuumed to give compound 1001 as a light yellow solid. 56 mg, 98%. MS (ESI) m/z (M+H)⁺ 891.

### 10.2 Synthesis of Compound 1002

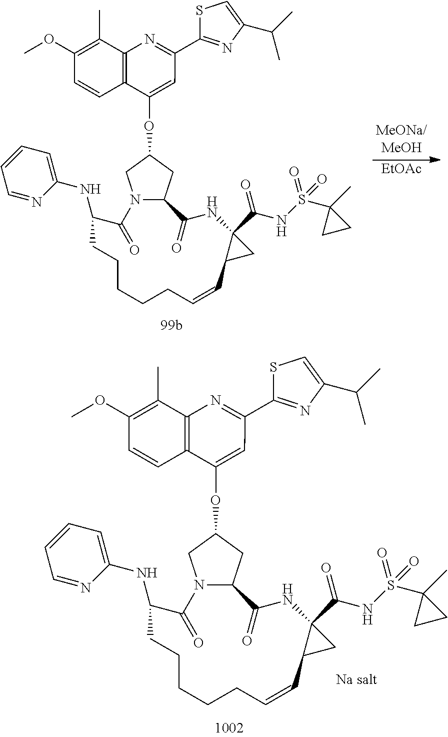

To the solution of compound 99b (50 mg, 0.06 mmol.) in EtOAc (2 mL) was added MeONa (3.2 mg, 0.06 mmol) and MeOH (0.5 mL) slowly at 0° C., the mixture was stirred at 0° C. for 1 h. Then the solvent was evaporated to give compound 1002 as a light yellow solid. 53 mg, 100%. MS (ESI) m/z (M+H)⁺ 856.2.

### 10.3 Synthesis of Building Block

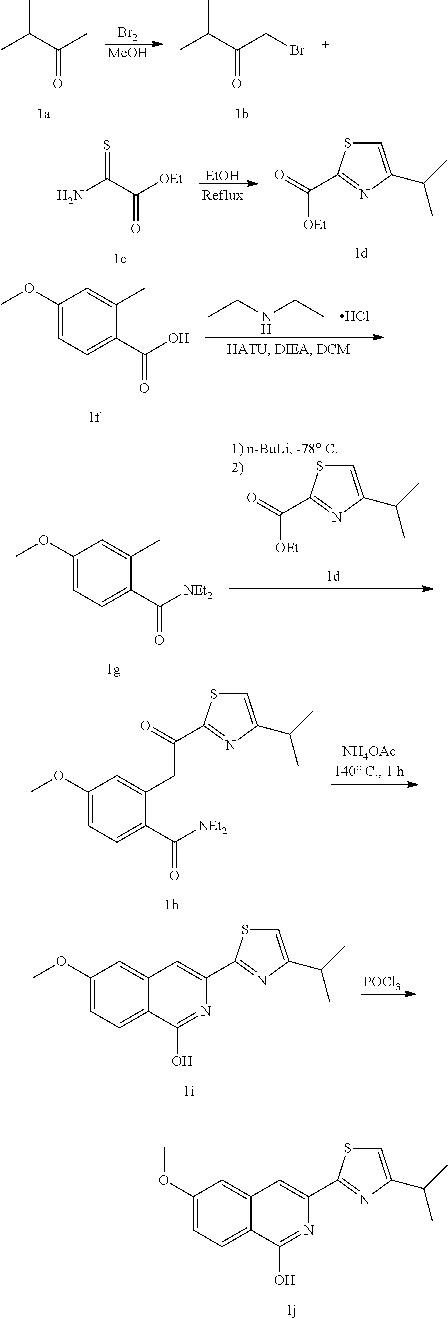

To a solution of compound 1a (5 g, 58 mmol) in methanol (30 mL) was added bromine (9.26 g, 58 mmol). The reaction was allowed to proceed below 10° C. Stirring was then continued at room temperature for 30 min before water (18 mL) was added. After 15 min, the mixture was diluted with water (50 mL) and extracted four times with diethyl ether. The ether extracts were successively washed with 10% Na₂CO₃ solution, water, brine, and dried over Na₂SO₄, concentrated in vacuo to give compound 1b as a liquid (5 g, 52%). ¹H NMR (400 MHz, CDCl₃) δ 3.99 (s, 2H), 3.05-2.95 (m, 1H), 1.17 (d, J=6.8 Hz, 6H).

To a boiling solution of compound 1c (4 g, 30 mmol) in ethanol (30 mL) was added compound 1b(5 g, 30 mmol) dropwise during 15 min. The solution was refluxed for 1 hour. After the solution was added to 100 mL of ice-cold water and basified with concentrated ammonia solution. This mixture was extracted twice with EtOAc. The organic phase was washed with brine, dried Na₂SO₄, and evaporated under reduced pressure. The crude product was purified by column chromatography with dichloromethane to give 4.4 g of the target product 1d (73%). ¹H NMR (CDCl₃): 7.12 (s, 1H), 4.47-4.37 (m, 2H), 3.23-3.14 (m, 1H), 1.37 (t, J=9.2 Hz, 3H), 1.27 (d, J=9.2 Hz, 6H).

A flask (100 mL) was charged with compound 1f (4.4 g, 26.5 mmol) and CH₂Cl₂ (50 mL). To the mixture was added HATU (15 g, 40 mmol) and DIEA (13.7 g, 106 mmol), diethylamine hydrochloride (3.49 g, 31.8 mmol). The resulting mixture was stirred at r.t. for 12 hrs. After the S.M was consumed, the mixture was diluted with EtOAc (100 mL), washed with water and brine, dried over sodium sulfate, concentrated in vacuo to give a yellow oil. It was isolated with silica gel column chromatography (eluted with PE:EA=3:1) to afford 1 g as a yellow oil (5.5 g, 94%).

To a solution of compound 1 g (300 mg, 1.36 mmol) in anhydrous THF (10 mL) at −78° C. under nitrogen was added n-BuLi (2.5M solution in hexane, 1.11 mL, 2.78 mmol) dropwise. The solution was kept at −78° C. for additional 30 min. Then, a solution of compound 1d (320 mg, 1.6 mmol) in anhydrous THF (3 mL) was added dropwise. After stirring for 2 hrs, the reaction was partitioned between ice-cold water and EtOAc. Purification by column chromatography afforded compound 1 h (400 mg, 79%) as a yellow oil.

A mixture of compound 1 h (190 mg, 0.51 mmol) and ammonium acetate (1.17 g, 15.2 mmol) was heated at 140° C. by microwave for 15 min, then, cooled down to room temperature. The reaction mixture was partitioned between ice-cold water and CH₂Cl₂, dried and filtered over silica to give compound 1i (100 mg, 65%) as a white solid.

A flask was charged with compound 1i (30 mg) and POCl₃ (2 mL). Heated under reflux for 4 hours. The TLC show the reaction was completed. The mixture was poured into ice-water. Neutralized by ammonia and extracted with EtOAc. Dried over sodium sulfate, concentrated in vacuo to give compound 1j (10 mg, 31%).

10.4 Preparation of compound 1003

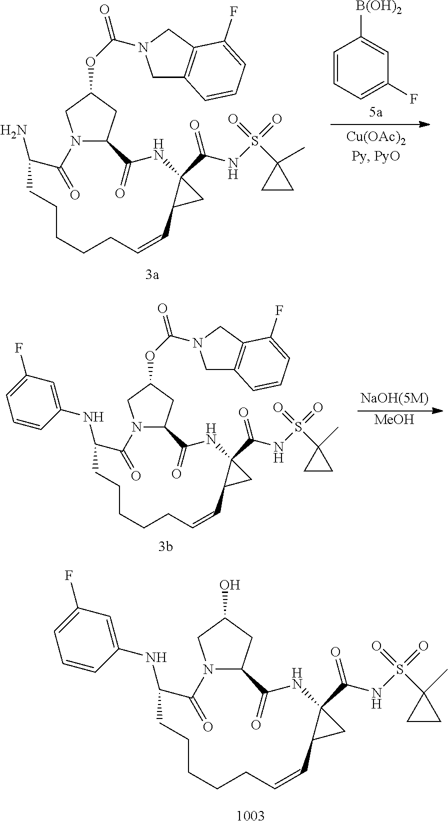

A mixture of compound 3a (350 mg, 0.54 mmol.), boronic acid 5a (228 mg, 1.63 mmol.), Cu(OAc)₂ (295 mg, 1.63 mmol.), pyridine (43 mg, 5.4 mmol.), pyridine N-oxide (513 mg, 5.4 mmol.) and molecular sieves 4 Å in dichloromethane (20 mL) was stirred for 12 h at room temperature under an atmosphere of oxygen. The reaction was monitored by LCMS. After completion of the reaction, the reaction mixture was diluted with ethyl acetate (30 mL) and filtered. The filtrate was washed with brine, dried over anhydrous sodium sulfate, concentrated in vacuo. The residue was purified by flash column chromatography to give compound 1003 (170 mg, 42.6%).

10.5 Synthesis of compounds 1004

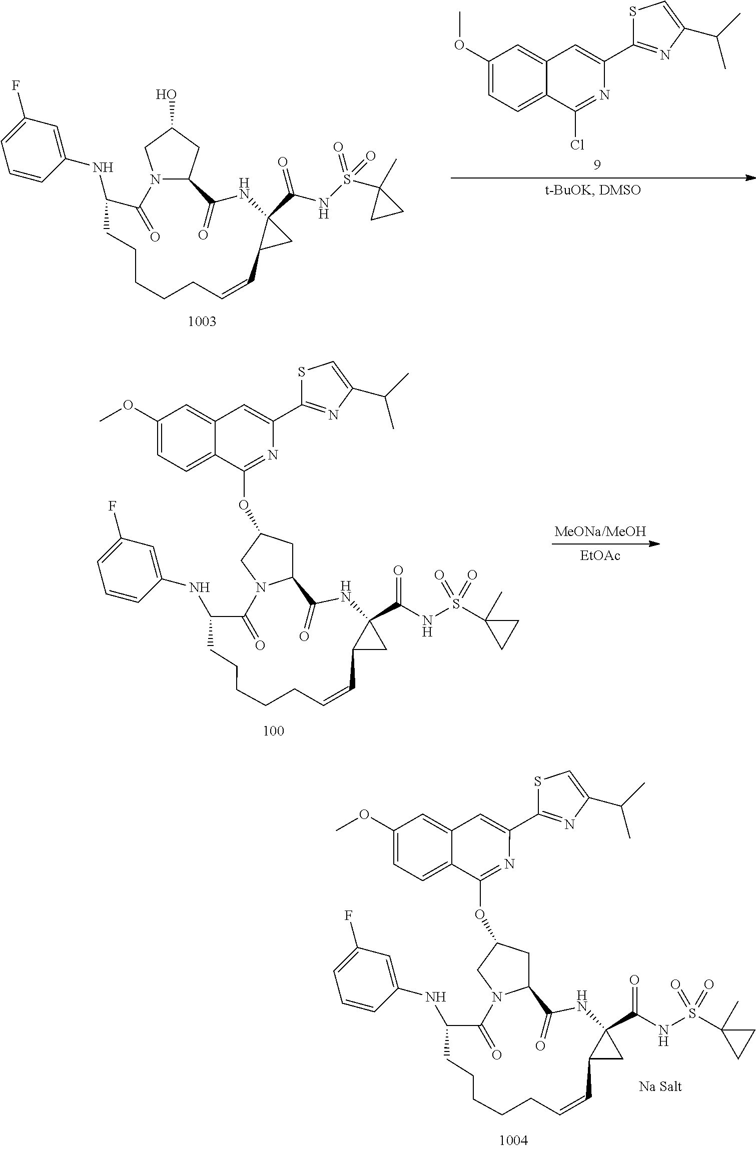

A flask was charged with 1003 (96 mg, 0.156 mmol), t-BuOK (87 mg, 0.78 mmol) and DMSO (2 mL) under nitrogen and stirred at room temperature for 20 min. Then compound 9 (74 mg, 0.234 mmol) was added and the mixture was stirred for 12 hrs. LCMS shows the reaction completed and the reaction was quenched by ice-water, acidified with aq. HCl (1 N) to pH=5-6, and extracted with EtOAc for three times. The combined organic layers, were dried over anhydrous sodium sulfate and concentrated in vacuo to give crude product. The crude product was purified by prep-HPLC to afford compound 100 (30 mg, 21%). MS (ESI) m/z (M+H)⁺ 859.2.

To a solution of compound 100 (62.0 mg, 0.07 mmol) in EtOAc was added MeONa (1 eq.)/MeOH solution (30%) slowly at 0° C., the mixture was stirred at 0° C. for 1 h. Then the solvent was evaporated in vacuo to give Na salt of compound 100 as a light yellow solid compound 1004. 63.6 mg, 100%. MS (ESI) m/z (M+H)⁺ 858.9.

10.6 Synthesis of compounds 1005 and 1006

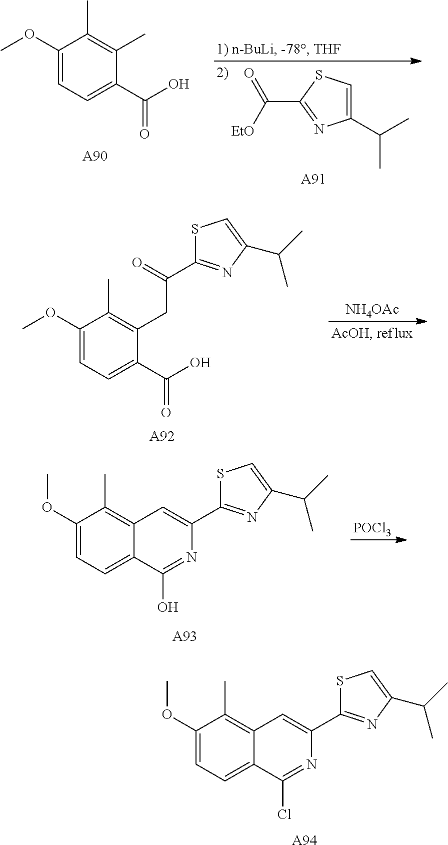

Preparation of precursor: To a solution of compound A90 (1.8 g, 10 mmol) in anhydrous TRF (40 mL) at −78° C. under nitrogen was added n-BuLi (2.5M solution in hexane, 12 mL, 30 mmol) dropwise. The solution was kept at −78° C. for additional 30 min. Then, a solution of compound A91 (2.4 g, 12 mmol) in anhydrous TRF (10 mL) was added dropwise. After addition was completed, the reaction mixture was allowed to warm slowly to r.t. and stirred for 12 hours. LCMS monitored the reaction. The reaction was quenched with saturated aq. NH₄Cl at 0° C., and adjusted to pH=4-5, the mixture was extracted with EtOAc (40 mL×3), the combined extracts was washed with brine, dried over sodium sulfate, concentrated in vacuo. The residue was purified by reverse phase HPLC to afford compound A92 (750 mg, 22.5%) as a white solid. ¹H NMR (400 MHz, DMSO-d₆) δ 12.45 (brs, 1H), 7.84 (d, J=8.8 Hz, 1H), 7.79 (s, 1H), 7.00 (d, J=8.8 Hz, 1H), 4.89 (s, 2H), 3.87 (s, 3H), 3.21-3.13 (m, 1H), 2.08 (s, 3H), 1.32 (d, J=6.8 Hz, 6H).

To a mixture of compound A92 (250 mg, 0.75 mmol) and acetic acid (2 mL) was added NH₄OAc (2 g, 26.25 mmol), the resulting mixture was heated at 130° C. for 5 hours. LCMS monitored the reaction. When the material was consumed, the mixture was cooled to r.t., diluted with water and extracted with EtOAc (20 mL×3), the combined extracts was washed with saturated aq. NaHCO₃ and brine, dried over sodium sulfate, concentrated in vacuo. The residue was isolated with silica gel column chromatography (eluted with PE:EA=4:1) to afford compound A93 (230 mg, 88%) as a white solid. ¹H NMR (400MHz, CDCl₃) δ 9.61 (brs, 1H), 8.25 (d, J=8.8 Hz, 1H), 7.03 (d, J=8.8 Hz, 1H), 7.02 (s, 1H), 6.87 (s, 1H), 3.88 (s, 3H), 3.08-3.00 (m, 1H), 2.34 (s, 3H), 1.27 (d, J=6.8 Hz, 6H).

A flask was charged with compound A93 (300 mg, 096 mmol) and POCl₃ (20 mL), the mixture was heated under reflux for 4 hours. TLC shows the reaction was completed. After cooling to r.t., most of POCl₃ was removed under reduced pressure. The residue was diluted with ice-water, neutralized with saturated aq. NaHCO₃ and extracted with EtOAc (30 mL×3), the combined extracts was washed with brine, dried over sodium sulfate, concentrated in vacuo to give compound A94 as offwhite solid (220 mg, 68%).

Preparation of Compound 1005:

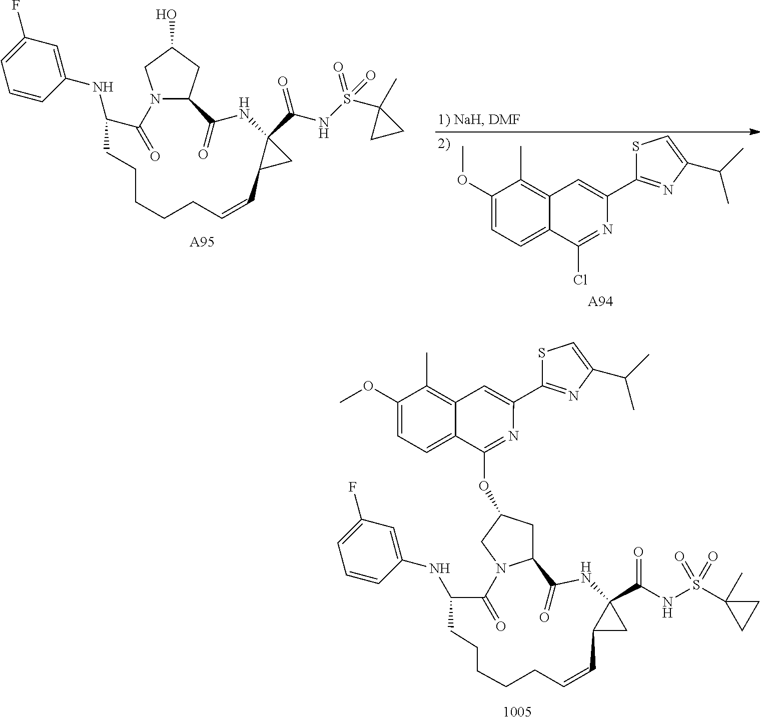

A dry and nitrogen purged flask was charged with compound A95 (200 mg, 0.347 mmol) and DMF (6 mL), to the resulting solution was added NaH (60% dispersion in mineral oil, 140 mg, 3.47 mmol) in portions. The mixture was stirred at r.t. for 1 hour, then compound A94 (127 mg, 0.382 mmol) was added, the stirring continued for 12 hours. LCMS showed the reaction was completed. The reaction was quenched by adding water, the aqueous layer was acidified with 1 N. HCl to pH≈5-6, and extracted with EtOAc (30 mL×3), the combined extract was washed with brine, dried over sodium sulfate, concentrated in vacuo. The residue was purified by preparative HPLC to afford compound 1005 (60 mg, 20%) as offwhite solid. MS (ESI) m/z (M+H)⁺ 873.3.

Preparation of Compound 1005S:

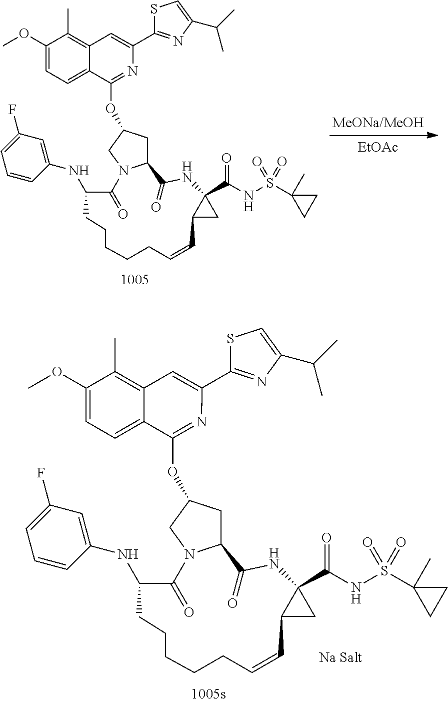

To a solution of compound 1005 (60 mg, 0.07 mmol) in EtOAc (5 mL) was added MeONa (1 eq.)/MeOH solution (30%) slowly at 0° C., the mixture was stirred at 0° C. for 1 h. Then the solvent was evaporated in vacuo to give the Na salt of compound 1005 (compound 1005S) as an offwhite solid (63 mg, 100%). MS (ESI) m/z (M+H)⁺ 873.4.

### Example 11

### 11.1 Synthesis of Compounds 1101 and 11015

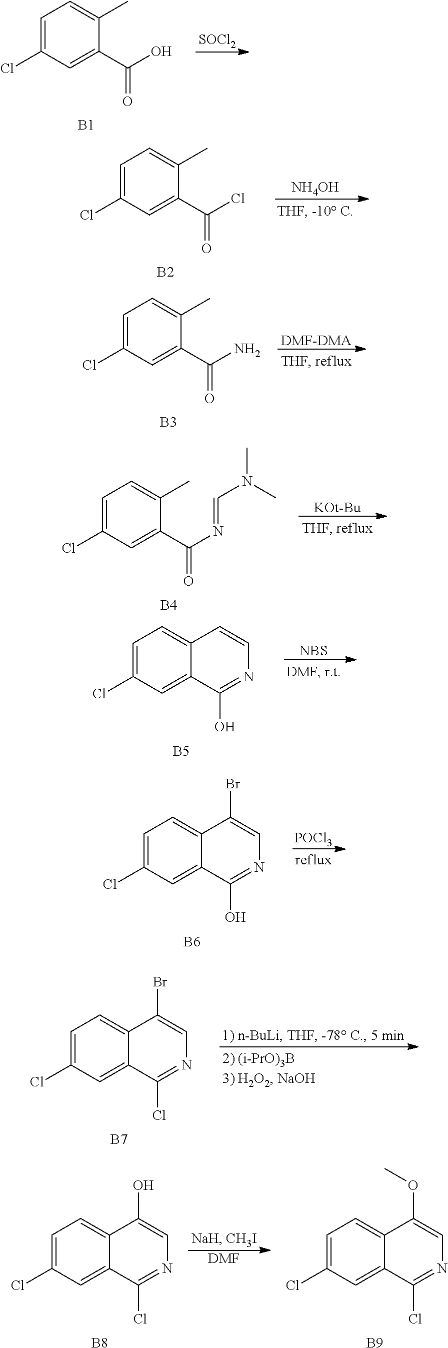

Preparation of precursor: A slurry of compound B1 (20 g, 0.18 mol) in thionyl chloride (42.3 mL, 0.54 mol) was heated slowly to a gentle reflux and maintained at this temperature for 2 hrs. The reaction mixture was then cooled to r.t. and the excess thionyl chloride was removed in vacuo. The residue was taken up in anhydrous DCM (50 mL), and the solvent then removed in vacuo. The resulting product was then dissolved in anhydrous TRF (80 mL), the resulting solution was used directly in next step.

To a solution of 30% ammonia (70 mL) in water (250 mL), cooled by a salt-ice bath (−10° C.), was added a solution of compound B2 in TRF (0.18 mol) dropwise. After the addition was complete, the resulting reaction mixture was stirred at −10° C. for 1 h. The reaction mixture was allowed to warm to room temperature and decanted. The remaining solid in the reaction vessel was then triturated with water (50 mL). This process of trituration and decanting was then repeated. The remaining solid was filtered and the filter cake was collected. The solid was dried in vacuo to yield compound B3 as a white crystal (16.5 g, 54%).

A mixture of compound B3 (16.5 g, 0.1 mol), DMF-DMA (16 mL, 0.12 mol), and anhydrous THF (200 mL) was heated to reflux and maintained at this temperature for 2 hrs. The reaction mixture was then cooled to room temperature and the volatiles were removed in vacuo. The resulting residue was recrystallized from n-hexane (200 mL) to yield compound B4 as white needles (19.5 g, 87%). ¹H NMR (400 MHz, CDCl₃): δ 8.50 (s, 1H), 7.98 (d, J=4.0 Hz, 1H), 7.20 (d, J=8.0 Hz, 1H), 7.06 (d, J=4.0 Hz, 1H), 3.12 (d, J=4.0 Hz, 6H), 2.5 (s, 3H).

A mixture of compound B4 (19.5 g, 87 mmol) and KOtBu (19.5 g, 174 mmol) in THF (250 mL) was heated to reflux and stirred at this temperature for 2 hrs. The volume of the reaction mixture was then reduced by distilling off approximately 100 mL of solvent. The resulting solution was then carefully poured into water (1 L) and the resulting mixture was then filtered, and the collected solid was washed thoroughly with water, dried in vacuo overnight to afford compound B5 as offwhite powder (9.8 g, 63%).

A flask was charged with compound B5 (9.84 g, 54.8 mmol), NBS (9.75 g, 54.8 mmol) and DMF (300 mL). The reaction mixture was stirred at room temperature for 2 hrs under nitrogen. The reaction was monitored with TLC. After completion of the reaction, the reaction mixture was diluted with water, extracted with EtOAc (150 mL×3), the organic layers were combined, washed with brine, dried over anhydrous sodium sulfate, the solvent was removed under reduced pressure to provide compound B6 (6.8 g, 48%), which was used in next step without further purification. ¹H NMR (300 MHz, CDCl₃): δ 8.27 (d, J=2.0 Hz, 1H), 7.88 (d, J=8.8 Hz, 1H), 7.81 (dd, 8.4 Hz, 1H), 7.51 (s, 1H).

A heterogeneous solution of compound B6 (6.8 g, 26.4 mmol) in POCl₃ (80 mL) was slowly heated to reflux for 4 hrs. The reaction mixture was then cooled to room temperature and concentrated in vacuo to remove excess POCl₃. The resulting residue was taken up into ice-water, and neutralized carefully with NaHCO₃ until the mixture was slightly basic (pH=8). The aqueous layer was extracted with EtOAc (100 mL×3), the organic layers were combined, washed with brine, dried over anhydrous sodium sulfate and concentrated in vacuo. The crude product was purified by column chromatography to afford compound B7 (7.0 g, 95.8%).

To a solution of compound B7 (1.0 g, 3.6 mmol) in THF at −78° C. was added n-BuLi (2.5 M in hexane, 11.5 mL, 28.6 mmol) dropwise via syringe over 15 min. The resulting solution was stirred for 10 min, after that, (i-PrO)₃B (3 mL, 7.2 mmol) was added dropwise via syringe over 10 min. The resulting reaction mixture was stirred for 6 hrs from −78° C. to r.t. After checking the reaction by TLC for completion, the reaction mixture was cooled to −78° C. and a solution of H₂O₂ (30%, 4 mL, 38.8 mmol) was added dropwise via syringe over 10 min, followed by addition of NaOH (144 mg, 3.6 mmol). The cooling bath was removed, and the reaction mixture was allowed to warm to room temperature and stirred at room temperature for 2 hrs. After confirming the completion of the reaction by TLC, the reaction mixture was then cooled to −40° C., and a solution of Na₂SO₃ (4.5 g) in 20 mL of water was added dropwise via syringe as a means to quench excess H₂O₂ over 30 min. The resulting solution was then acidified with aq. HCl (6 M) at 0° C. till pH=6, then diluted with EtOAc and decanted to a separatory funnel. The combined organic layers were washed with brine, dried over anhydrous sodium sulfate and the solvent was removed under reduced pressure. The crude product was washed by DCM, the solid was collected by filtration, dried to give compound B8 (300 mg, 39%).

To a solution of compound B8 (180 mg, 0.84 mmol) in DMF (8 mL) was added NaH (60%, 40 mg, 1.0 mmol) portion-wise. The mixture was stirred at 0° C. for 30 min under nitrogen, then CH₃I (0.07 mL, 1.26 mmol) was added. The stirring was continued for 3 hrs at 25° C. The reaction was monitored by TLC. After completion of the reaction, the reaction mixture was poured into ice water, neutralized with aq. HCl (1 M), extracted with EtOAc (40 mL×3), the organic layer was combined and washed with brine, dried over anhydrous sodium sulfate, the solvent was removed under reduced pressure. The crude product was purified with prep-TLC to give compound B9 (120 mg, 62.5%). ¹HNMR (400MHz, CDCl₃): δ 8.18 (d, J=2.0 Hz, 1H), 8.16 (d, J=8.8 Hz, 1H), 7.80 (s, 1H), 7.67 (dd, J=2.0, 12 Hz, 1H), 4.05 (s, 3H).

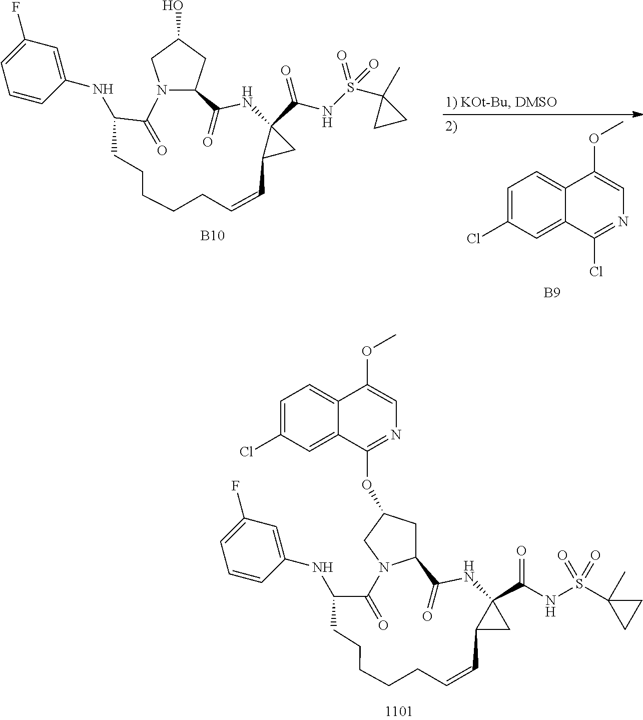

Preparation of compound 1101: A flask was charged with compound B10 (170 mg, 0.295 mmol) and DMSO (4 mL), the solution was purged with nitrogen and then KOt-Bu (166 mg, 1.475 mmol) was added thereto. The mixture was stirred at room temperature for 1 hour. Then compound B9 (73 mg, 0.32 mmol) was added and the mixture was stirred for 12 hrs at room temperature. LCMS shows the reaction completed and the reaction was quenched by ice-water, acidified with aq. HCl (1 M) to pH=5-6, extracted with EtOAc (40 mL×3), the organic layer was combined and washed with brine, dried over anhydrous sodium sulfate, concentrated in vacuo to give crude product. It was purified with preparative HPLC to give compound 1101 (58 mg, 25.6%). MS (ESI) m/z (M+H)⁺ 768.2.

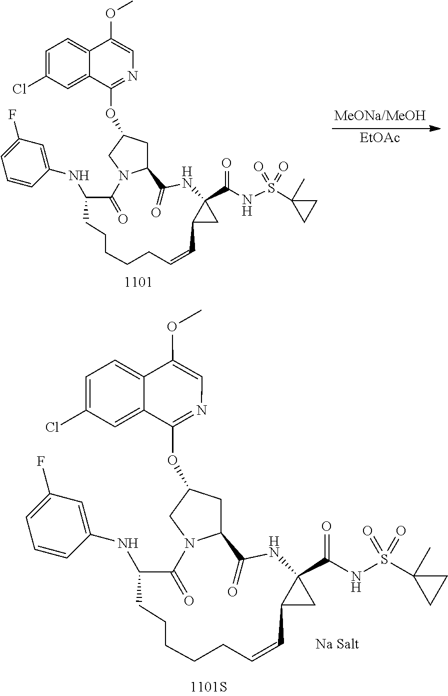

Preparation of compound 1101S: To a solution of compound 1101 (58 mg, 0.0755 mmol) in EtOAc (2 mL) was added MeONa (1 eq.)/MeOH solution (30%) slowly at 0° C., the mixture was stirred at 0° C. for 1 h. Then the solvent was evaporated in vacuo to give Na salt of 1101 (compound 1101S) (59.5 mg, 100%). MS (ESI) m/z (M+H)⁺ 768.2.

### Example 12

### 12.1: Synthesis of Compounds 1251-1253

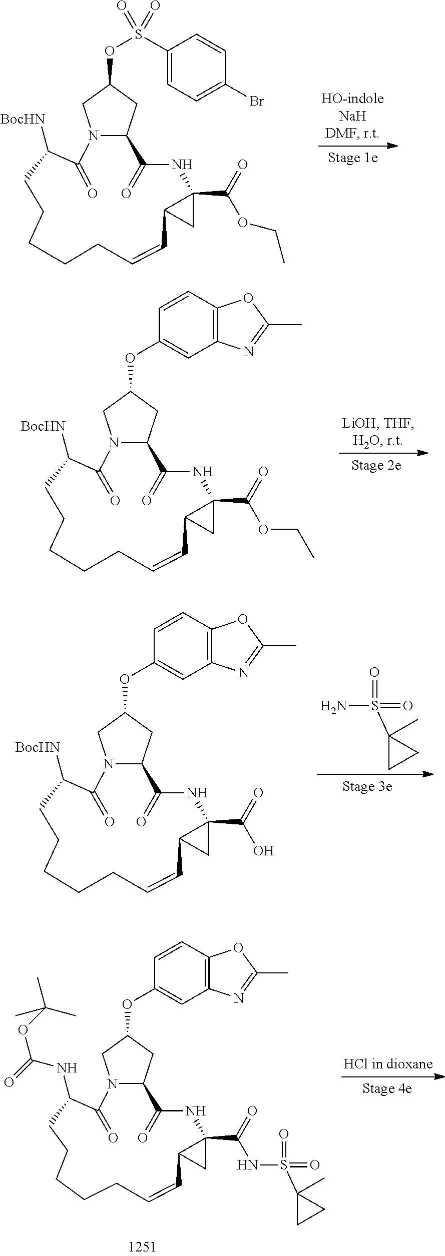

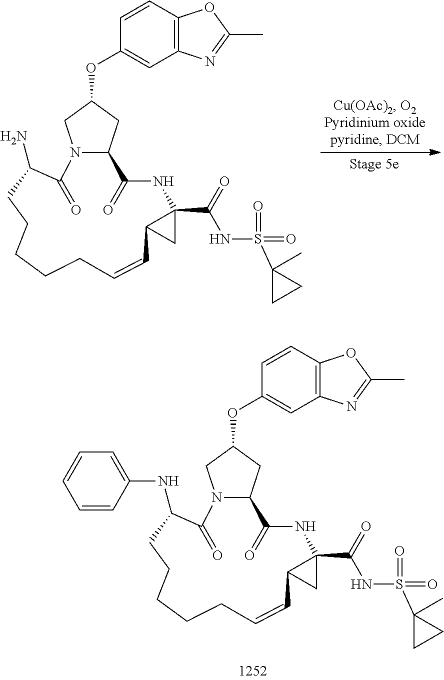

Compound 1251 was prepared following the method described for making compound 702 in section 7.2 above. 14.5 mg (14%) as a white solid after preparative HPLC. ¹HNMR (500 MHz, CDCl₃) δ ppm 10.07 (s, 1H) 7.37 (d, J=8.85 Hz, 1H) 7.15 (s, 1H) 6.86 (dd, J=8.70, 2.44 Hz, 1H) 6.79 (br. s., 1H) 5.72 (q, J=8.75 Hz, 1H) 5.18-5.28 (m, 1H) 5.09 (br. s., 1H) 5.00 (t, J=9.54 Hz, 1H) 4.58 (t, J=7.93 Hz, 1H) 4.24-4.40 (m, 2H) 3.83-3.95 (m, 1H) 2.63 (s, 3H) 2.55 (dd, J=7.93, 3.20 Hz, 3H) 2.30 (q, J=8.65 Hz, 1H) 1.85-1.97 (m, 2H) 1.74-1.85 (m, 2H) 1.66 (br. s., 4H) 1.51-1.55 (m, 1H) 1.49 (s, 3H) 1.46-1.51 (m, 1H) 1.42-1.44 (m, 1H) 1.37 (s, 9H) 1.24-1.34 (m, 2H) 0.78-0.89 (m, 2H). LC-MS: purity 99% (UV), t_{R} 4.71 min m/z [M+H]⁺ 714.30 (MET/CR/1416).

Compound 1252 was prepared following the method described for making compound 802 in section 8.2 above. 10.4 mg (33%) as a off-white solid after preparative HPLC. ¹H NMR (500 MHz, DMSO-d⁶) 8 ppm 10.88 (br. s., 1H) 9.01 (br. s., 1H) 7.58-7.62 (m, 1H) 7.29-7.31 (m, 1H) 6.91-6.95 (m, 1H) 6.86-6.91 (m, 2H) 6.45-6.52 (m, 2 H) 5.58-5.68 (m, 2H) 5.27-5.32 (m, 1H) 5.00-5.05 (m, 1H) 4.41 (dd, J=8.85, 7.93 Hz, 1H) 4.18-4.28 (m, 2H) 3.85-3.91 (m, 1H) 2.57-2.60 (m, 3H) 2.23-2.32 (m, 2H) 1.71-1.87 (m, 2H) 1.56-1.60 (m, 1H) 1.46-1.51 (m, 2H) 1.39-1.44 (m, 3H) 1.37-1.39 (m, 3H) 1.25-1.31 (m, 2H) 1.19-1.25 (m, 3H) 1.13-1.19 (m, 2H) 0.82-0.92 (m, 3H). LC-MS: purity 99% (UV), t_{R} 4.80 min m/z [M+H]⁺ 690.05 (MET/CR/1416).

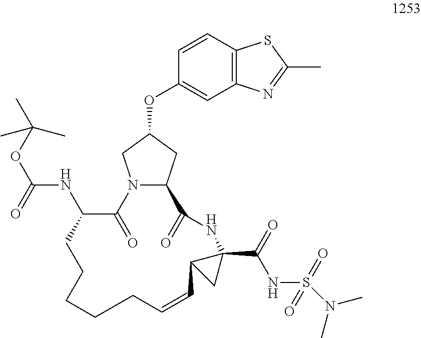

Compound 1253 was prepared following the method described for making compound 702 in section 7.2 above. 8.2 mg (10%) as a white solid after preparative HPLC. ¹HNMR (500 MHz, CDCl₃) δ ppm 9.99 (br. s., 1H) 7.69 (d, J=8.54 Hz, 1H) 7.35-7.47 (m, 1H) 6.86-7.02 (m, 2H) 5.74 (q, J=8.80 Hz, 1H) 5.20-5.27 (m, 1H) 5.09-5.20 (m, 1H) 5.01 (t, J=9.38 Hz, 1H) 4.54-4.71 (m, 1H) 4.23-4.40 (m, 2H) 3.89-4.05 (m, 1H) 2.88 (s, 6H) 2.83 (s, 3H) 2.46-2.64 (m, 3H) 2.26 (q, J=9.00 Hz, 1H) 1.74-1.95 (m, 4H) 1.52-1.64 (m, 2H) 1.44 (br. s., 3H) 1.35 (s, 9H) 1.29-1.31 (m, 2H). LC-MS: purity 94% (UV), t_{R} 4.77 min m/z [M+H]⁺ 719.30 (MET/CR/1416).

### Example 14

### 14.1 Synthesis of Compound 1401

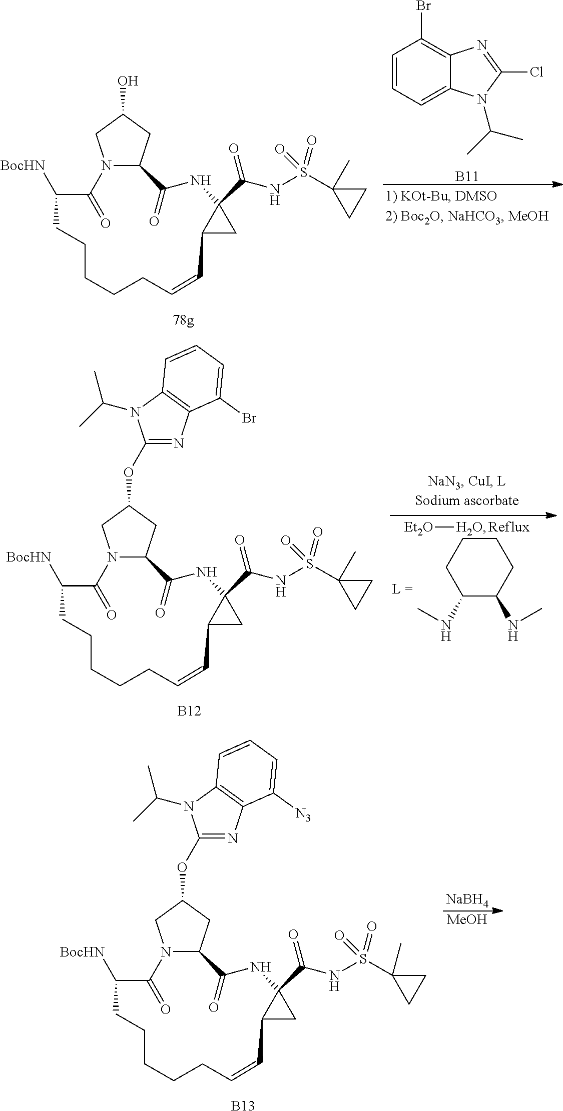

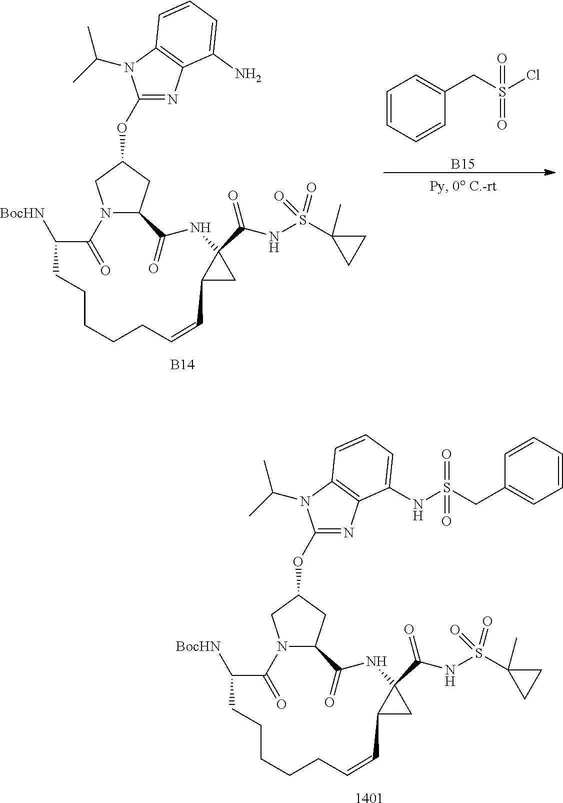

To a solution of compound 78g (200 mg, 0.34 mmol, 1 eq.) in 2 mL of DMSO was added KOt-Bu (192 mg, 1.72 mmol, 5 eq.) in portions at ambient temperature, then the mixture was stirred for 2 hrs at ambient temperature. After that, compound B11 (103 mg, 0.38 mmol, 1.1 eq.) was added, the resulting mixture was stirred at r.t. for 20 hrs, the reaction was monitored by LCMS. The de-Boc product was detected, the mixture was cooled by ice water, acidified by aq. HCl (2 M) to pH=7-8. Then Boc₂O (74 mg, 0.34 mmol, 1 eq.) and NaHCO₃ (32 mg, 0.38 mmol, 1.1 eq.) were added. The mixture was stirred for another 2 hrs, extracted by ethyl acetate (100 mL×3), the organic layers were combined, washed by brine, dried over anhydrous sodium sulfate, concentrated under reduced pressure, the residue was purified by prep-TLC to give compound B12 (180 mg, yield 79%).

Compound B12 (180 mg., 0.22 mmol, 1 eq.), NaN₃ (29 mg, 0.32 mmol, 2 eq.), ligand (15.6 mg, 0.11 mmol, 0.5 eq.), CuI (42 mg, 0.22 mmol, 1 eq.), sodium ascorbate (44 mg, 0.22 mmol, 1 eq) and 2 mL of EtOH—H₂O (7:3) were introduced into a round-bottom flask equipped with a stirring bar and a reflux condenser. After the flask was degassed with nitrogen, the reaction mixture was stirred under reflux for 8 hrs, the reaction was monitored by LCMS. After completion of the reaction, the mixture was cooled to room temperature, extracted by ethyl acetate (30 mL×3), the organic layers were combined, washed by brine, dried over anhydrous sodium sulfate, concentrated under reduced pressure, the residue was purified by prep-TLC to give compound B13 (30 mg, yield 17.4%).

To a solution of compound B13 (30 mg, 0.038 mmol, 1 eq) in 3 mL of methanol was added NaBH₄ (14.5 mg, 0.38 mmol, 30 eq). The solution was stirred at room temperature. TLC analysis showed the reaction completed. All the volatiles were removed under reduced pressure. The residual was diluted with water, extracted with ethyl acetate (30 mL×3), the organic layers were combined, washed by brine, dried over anhydrous sodium sulfate, concentrated under reduced pressure, the residue was purified by prep-TLC to give compound B14 (20 mg, yield 69.7%).

To a solution of compound B14 (20 mg, 0.026 mmol, 1 eq.) in 2 mL of pyridine was added compound B15 (14.8 mg, 0.078 mmol, 3 eq.) at 0° C. The solution was stirred for 2 h at 0° C., then allowed to warm to room temperature, and continued to stir for another 18 hrs. LCMS analysis showed the reaction completed. The reaction mixture was diluted with ethyl acetate, washed with aq. HCl (1 N), saturated aqueous NaHCO₃ and water. The combined organic layer was dried over anhydrous sodium sulfate, filtered. The solvent was removed under reduced pressure, the residue was purified by prep-TLC to give compound 1401 (8.3 mg, yield 35.0%). MS (ESI) m/z (M+H)⁺ 910.5.

### 14.2 Synthesis of Compound 1402

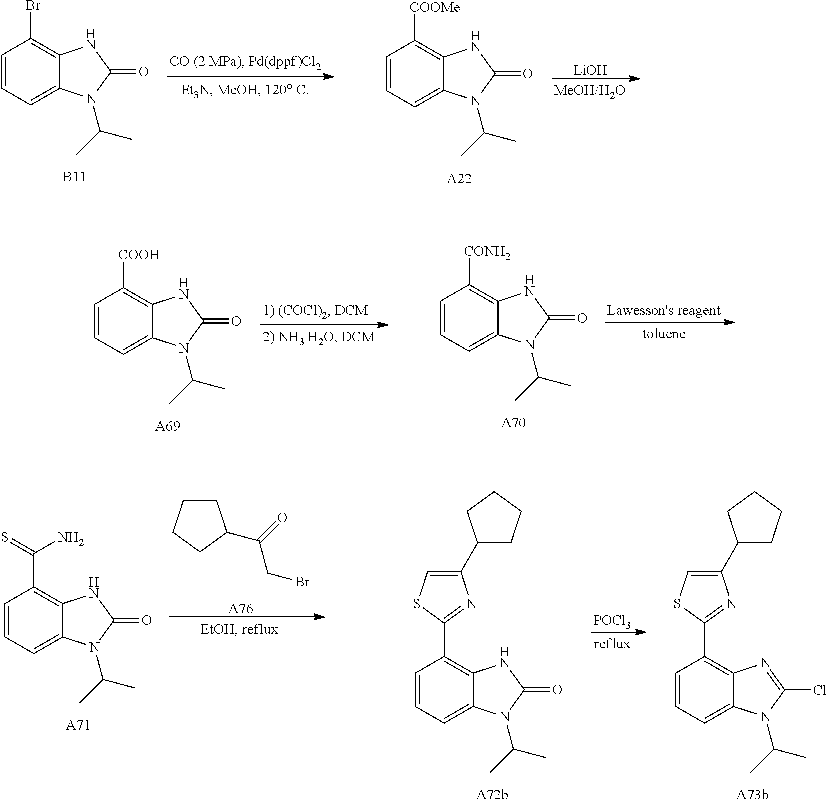

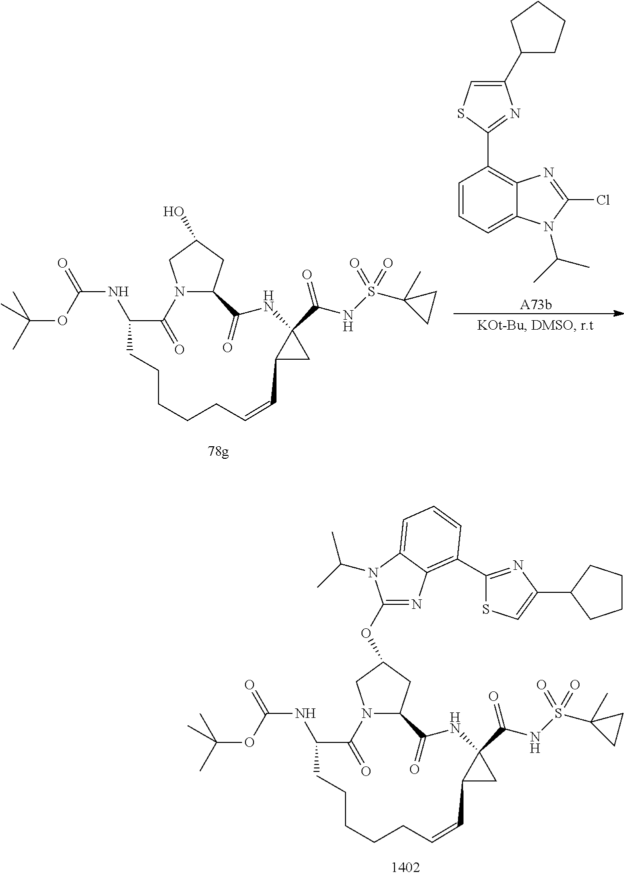

The autoclave was charged with compound B11 (10.85 g, 42.55 mmol), Pd(dppf)Cl₂ (3.1 g, 4.26 mmol), Et₃N (8.88 mL, 63.83 mmol) and MeOH (500 mL), then degassed with CO. The mixture was stirred at 120° C. under CO (2 MPa) for 2 days. The reaction mixture was filtered and the filtrate was concentrated under reduced pressure. The residue (16g) was used directly in next step without further purification. MS (ESI) m/z (M+H)⁺ 235.0.

To a solution of compound A22 (4.0 g, 17.1 mmol) in MeOH (40 mL) and water (30 mL) was added LiOH (4.0 g, 171 mmol). The mixture was stirred for 12 hrs at r.t. After that, the solvent was removed under reduced pressure, the aqueous layer was acidified to pH=3 with aq. HCl (2 M) and then extracted with EtOAc. The combined organic layer was washed with brine, dried over anhydrous Na₂SO₄ and concentrated to give a crude product compound A69, which was used directly in next step. (3.6 g, yield 96%).

To a solution of compound A69 (3.6 g, 16 mmol) in anhydrous DCM (80 mL) was added oxalyl chloride (2.7 g, 21 mmol) at 0° C., and followed by DMF (two drops) at 0° C. The mixture was stirred for 15 min at 0° C. and then stirred for 30 min at r.t. After completion of the reaction, the solvent was evaporated under reduced pressure to give a crude product. To a solution of the resulting product in anhydrous DCM (80 mL) was added ammonia (14 mL) and then the mixture was stirred for 12 hrs at r.t. After that, solids were filtered off, washed with DCM, and dried over vacuum freeze-drier to give a white solid, compound A70 which was used directly in next step (3.2 g, yield 91%). MS (ESI) m/z (M+H)⁺ 220.8.

A flask was charged with compound A70 (3.2 g, 14.6 mmol), Lawesson's reagent (3.0 g, 7.3 mmol) and anhydrous toluene (60 mL). The mixture was refluxed under nitrogen. After completion of the reaction, solids were filtered off and washed with EtOAc to give a yellow crude product compound A71, which was used directly in next step (2.14 g, yield 62%).

To a solution of compound A71 (3.35 g, 14.2 mmol) in EtOH (60 mL) was added compound A76 (3.4 g, 17.8 mmol). The mixture was refluxed under nitrogen. After removal of the solvent, the reaction mixture was diluted with EtOAc (60 mL), washed with water (50 mL) and brine (30 mL×2), dried over anhydrous Na₂SO₄, and then concentrated in vacuo. The residue was purified by column chromatography to give compound A72b (3.0 g, yield 64%). MS (ESI) m/z (M+H)⁺ 328.2.

Compound A72b (3.0 g, 9.17 mmol) was dissolved in POCl₃ (15 mL) and then the mixture was refluxed under nitrogen. After the reaction completion, The reaction mixture was taken up with ice-water, neutralized with ammonia under cooling, extracted with EtOAc (40 mL×3). The combined organic layer was washed with brine, dried over anhydrous Na₂SO₄ and concentrated to give a crude compound A73b, which was used directly in next step (3.1 g, yield 98%). MS (ESI) m/z (M+H)⁺ 345.9.

Compound 1402 was prepared following the general procedures described in Example 2. Yielded 250.2 mg, 16.3%, white solid. MS (ESI) m/z (M+H)⁺ 892.3.

### 14.3 Synthesis of Compound 1403

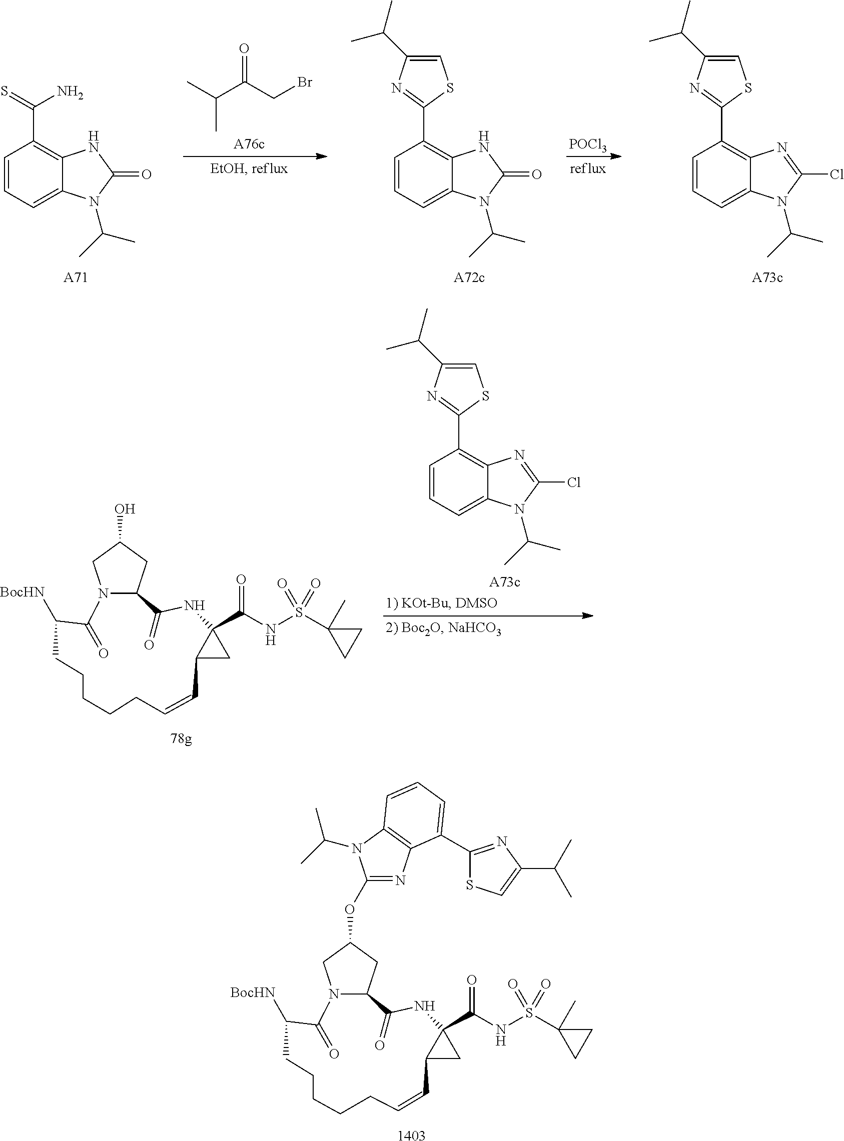

To a solution of compound A71 (2.0 g, 8.5 mmol) in EtOH (20 mL) was added compound A76c (2.8 g, 17.0 mmol). The mixture was refluxed under nitrogen. After completion of the reaction, the solvent was evaporated under reduced pressure. DCM (20 mL) was taken into and solids were filtered off and washed with DCM to give the compound A72c as a white solid (2.5 g, yield 97%). MS (ESI) m/z, 301.9.

Compound A72c (2.5 g, 8.3 mmol) was dissolved in POCl₃ (20 mL) and then the mixture was refluxed under nitrogen. After the reaction completion, the reaction mixture was taken into ice-water, neutralized with ammonia under cooling, extracted with EtOAc (40 mL×3). The combined organic layer was washed with brine, dried over anhydrous Na₂SO₄ and concentrated to give a crude product compound A73c, which was used directly in next step (1.8 g, yield 66%). MS (ESI) m/z 319.8

Compound 78g (500 mg, 0.86 mmol) was dissolved in DMSO (7 mL) and the solution was degassed with nitrogen. Then KOt-Bu (404 mg, 3.61 mmol) was added and the mixture was stirred for 1 h at r.t under nitrogen. Then compound A73c (275 mg, 0.86 mmol) was added and then the mixture was stirred for 12 h at r.t under nitrogen. After the material was consumed, the de-Boc product was detected by LCMS. The reaction was quenched with ice-water. The mixture was adjusted to pH=6˜7 with aq. HCl (0.1M) and then MeOH (4 mL), NaHCO₃ (87 mg, 1.03 mmol), Boc₂O (187 mg, 0.86 mmol) were added. The mixture was stirred for 2 h at r.t. After that, MeOH was evaporated under reduced pressure and the resulting mixture was acidified to pH=5-6 with aq. HCl (0.1M), extracted with EtOAc (20 mL×3). The combined organic layer was washed with brine, dried over anhydrous Na₂SO₄, and concentrated to give a crude product, which was purified by prep-HPLC to afford compound 1403 (320 mg, yield 43%). MS (ESI) m/z 866.4.

### 14.4 Synthesis of Compound 1404

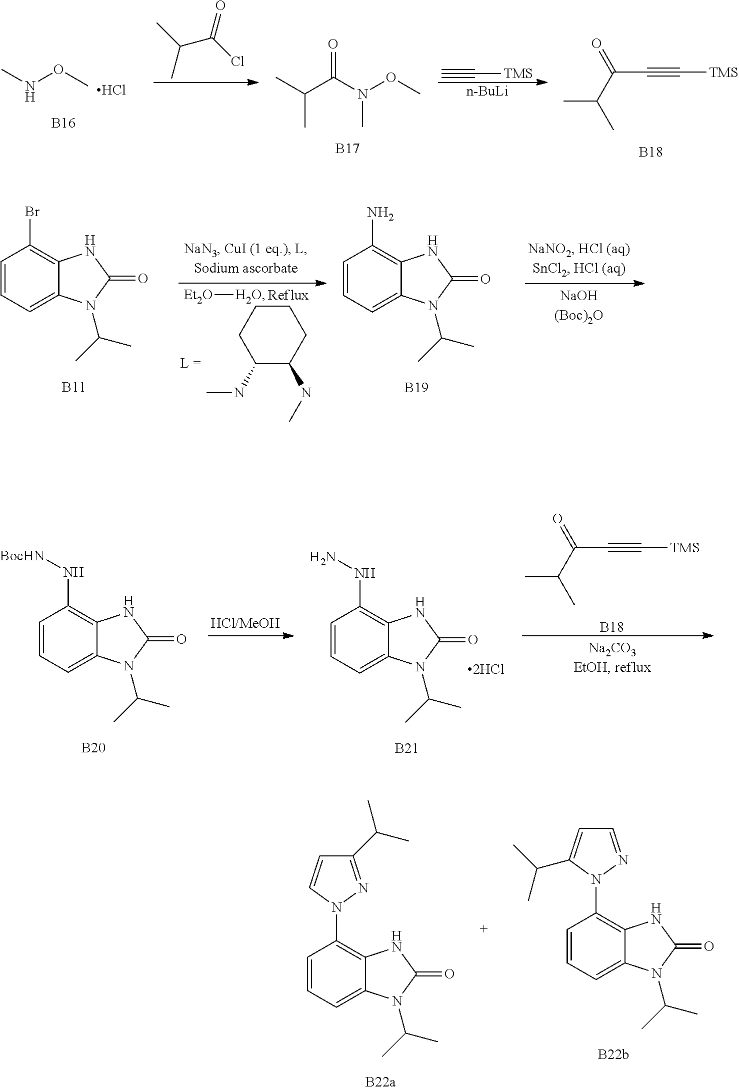

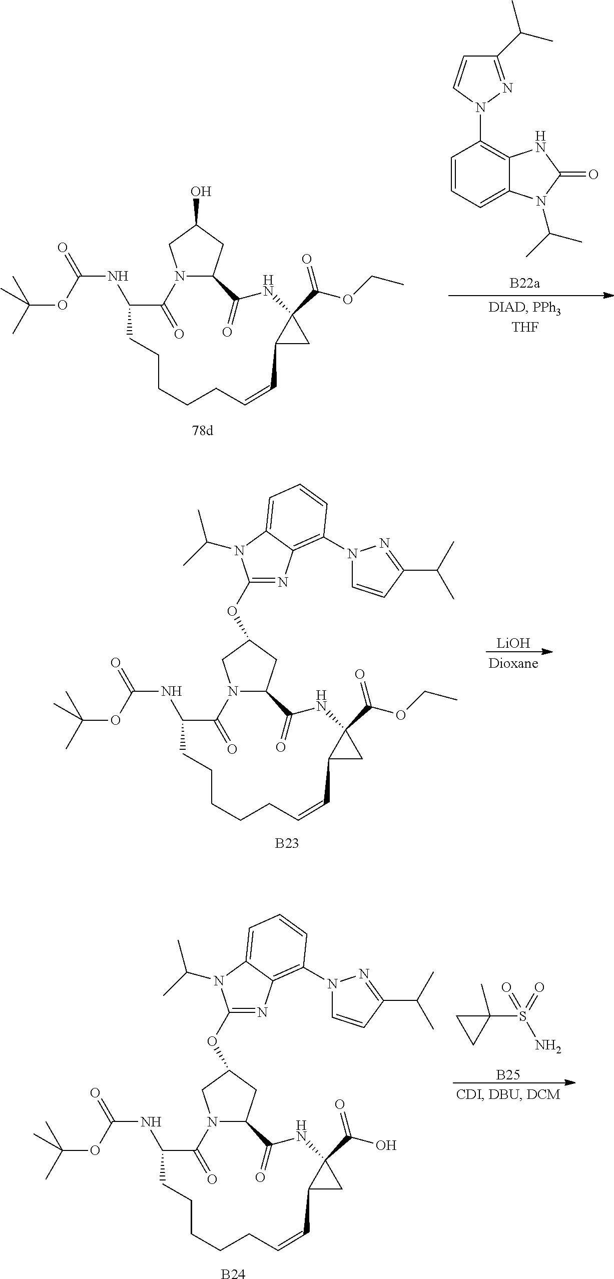

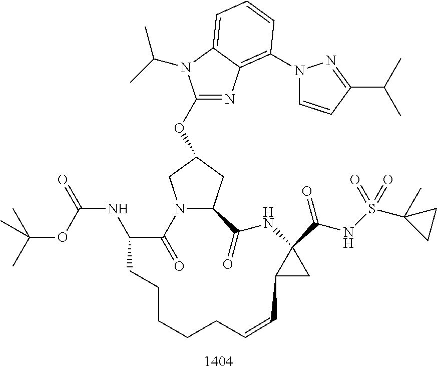

To a solution of N,O-dimethylhydroxylamine hydrochloride (5.0 g, 28.7 mmol) and TEA (8.8 mL, 63.2 mL) in anhydrous DCM (50 mL) was added isobutyryl chloride (3.1 mL, 28.7 mmol) at 0° C., the resulting mixture was allowed to warm to room temperature and stirred overnight. The reaction was detected by TLC. After the reaction completion, the mixture was concentrated in vacuo and filtered, the filtrate was washed with brine and extracted with EtOAc, and the organic layer was concentrated in vacuo to give a yellow residue. The residue was purified by column chromatography (petroleum ether: EtOAc=10:1). Compound B17 was obtained as light yellow oil (1.2 g, yield 32%). MS (ESI) m/z (M+H)⁺ 132.0.

Ethynyltrimethylsilane (1.0 g, 10.7 mmol) was dissolved in anhydrous THF (25 mL), the solution was cooled to −65° C., then n-BuLi (2.5 M solution in hexane, 4.8 mL, 11.7 mmol) was added dropwise. After that, the solution was slowly warmed to −30° C. for 1 h. Then the solution was recooled to −65° C. and compound B17 (1.4 g, 10.7 mmol) in THF (20 mL) was added through a syringe slowly. The reaction was slowly warmed to 0° C. and stirred for another 3 hrs. TLC showed the reaction complete. The reaction mixture was quenched with saturated aqueous NH₄Cl and extracted with EtOAc. The organic layer was dried over Na₂SO₄ and concentrated to give a light yellow oil. The crude compound B18 was pure enough for the next step (1.6 g, yield 89%).

To a mixture of compound B11 (2.0 g, 7.9 mmol), sodium ascorbate (779 mg, 3.9 mmol), CuI (1.5 g, 7.9 mmol), N,N′-Dimethyl-cyclohexane-1,2-diamine (110 mg, 0.8 mmol) in EtOH—H₂O (v/v=7:3) (50 mL) was added NaN₃ (1.1 g, 15.8 mmol). The resulting mixture was stirred at 60° C. for 6 hrs. The reaction was detected by TLC. After the reaction complete, the mixture was filtered, the filtrate was washed with brine and extracted with EtOAc, the organic layer was dried and concentrated to give a yellow residue, the residue was purified by prep-TLC (DCM: CH₃OH 20:1). Compound B19 was obtained as a light yellow solid (1.4 g, yield 93%). MS (ESI) m/z (M+H)⁺ 192.0.

To a suspension of compound B19 (564 mg, 3.0 mmol) in 1.5 mL of concentrated hydrochloric acid was added 1 mL of an aqueous solution of sodium nitrite (204 mg, 3.0 mmol), the mixture was stirred at 0° C. for 30 minutes. Tin (II) chloride (2.0 g, 9.1 mmol) was dissolved in 1 mL of concentrated hydrochloric acid, the solution was added to the reaction mixture at 0° C. After 1 h, the mixture was alkalified by aq. sodium hydroxide (12 N), followed by addition of ethyl acetate to the suspension. After addition of di-tert-butyl dicarbonate (2.2 g, 9.1 mmol), the mixture was stirred at room temperature for 2 h. The reaction mixture was extracted with ethyl acetate and washed with brine; the organic layer was dried over sodium sulfate and concentrated in vacuo. The residue was dried under vacuum to give 580 mg of compound B20 as yellow crystals. The crude compound was pure enough for the next step (790 mg, yield 89%). MS (ESI) m/z (M+H)⁺ 307.1.

Compound B20 (700 mg, 2.3 mmol) was dissolved in a solution of hydrogen chloride in methanol (4 M, 15 mL), the resulting solution was stirred at room temperature for 4 hours. The reaction was detected by LCMS. After the reaction complete, the reaction solution was concentrated in vacuo to give compound B21 as a dark red solid (623 mg, yield 97%). MS (ESI) m/z (M+Na)⁺ 229.0.

To a refluxing solution of compound B21 (460 mg, 1.7 mmol) and B18 (304 mg, 1.8 mmol) in EtOH was added slowly sat. aq of Na₂CO₃ (437 mg, 4.1 mmol). The mixture was stirred for additional 15 h, then diluted with H₂O and extracted with EtOAc. After drying the EtOAc layer and removal of the solvent under vacuum, a viscous oil was obtained. The oil was purified by prep-TLC (petroleum ether: EtOAc=2: 1). Compound B22a was isolated as a light yellow solid (130 mg, yield 28%), and an isomer B22b (140 mg, yield 29%) were isolated as well. Compound B22a: ¹H NMR (CDCl₃): 9.52 (s, 1H), 7.91 (d, 1H), 7.15 (m, 3H), 6.28 (d, 1H), 4.75 (m, 1H), 3.0 (m, 1H), 1.54 (d, 6H), 1.30 (d, 6H). MS (ESI) m/z (M+H)⁺ 285.0.

Macrocycle 78d (148 mg, 0.3 mmol), B22a (80 mg, 0.3 mmol), triphenylphosphine (274 mg, 1.0 mmol) and anhydrous tetrahydrofuran (20 mL) were charged into a 100 mL three neck flask. The reaction mixture was cooled on top of an ice bath and diisopropylazodicarboxylate (DIAD, 0.3 mL, 1.0 mmol) was added drop wise. The cooling bath was removed and stirring was continued at ambient temperature for a further 3 hrs by which time TLC and LCMS analyses showed full consumption of the starting material. Saturated aqueous sodium hydrogen carbonate (2 mL) was added and the reaction mixture stirred for a further 5 minutes, the reaction mixture was then extracted with DCM. The organic layer was combined, concentrated in vacuo. The residue was purified by prep-TLC (petroleum ether:EtOAc 1:1). Compound B23 was isolated as brown oil (120 mg, yield 53%). MS (ESI) m/z (M+H)⁺ 760.4.

To a solution of intermediate B23 (160 mg, 0.2 mmol) in 15 mL of dioxane was added 5 mL of aqueous lithium hydroxide (1 N, 5 mmol). The reaction was heated to 40° C. overnight. The reaction looked complete from LCMS. The mixture was neutralized by acetic acid and extracted with EtOAc. The organic extracts were combined, washed with saturated aqueous sodium hydrogen carbonate, and brine. The organic phase was dried in vacuo to give compound B24 as creamy foam (152 mg, yield 98%). MS (ESI) m/z (M+H)⁺ 732.3.

A solution of compound B24 (80 mg, 0.1 mmol) and CDI (106 mg, 0.6 mmol) in DCM (15 mL) was stirred at reflux for 4 hours under nitrogen atmosphere, then sulfonamide B25 (54 mg, 0.4 mmol) and DBU (121 mg, 0.8 mmol) was added. The resulting mixture was stirred at reflux overnight. The reaction solution was diluted with EtOAc, washed with brine and concentrated in vacuo. The final compound was purified by prep-HPLC to yield compound 1404 (71 mg, yield 77%). MS (ESI) m/z (M+H)⁺ 849.5.

### 14.5 Synthesis of Compounds 1405-1407

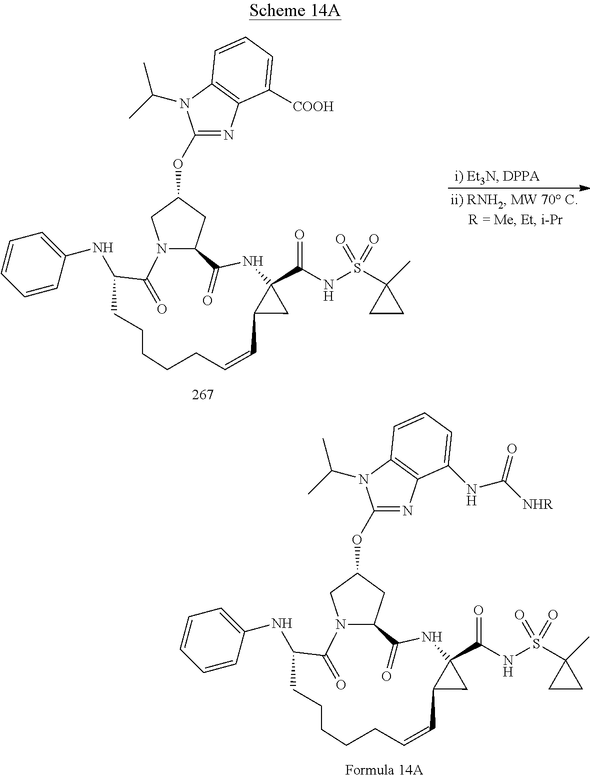

General procedure: To a solution of the macrocyclic intermediate 267 (50 mg, 0.066 mmol.) in anhydrous toluene was added TEA (33 mg, 0.33 mmol) and DPPA (54 mg, 0.20 mmol), the resulting mixture was stirred at 60° C. under nitrogen atmosphere. The reaction was detected by LCMS. After the reaction completed, the mixture was introduced into a microwave tube, then amine (0.20 mmol) was added and the tube was sealed. The mixture was heated at 70° C. by microwave for 20 min. Then the reaction mixture was diluted with ethyl acetate and washed with brine. The organic phase was gathered, dried over anhydrous sodium sulfate and concentrated in vacuo. The residue was purified by prep-HPLC to yield compound of Formula 14A. The following compounds were prepared according to Scheme 14A.

TABLE 14
Compounds prepared according to Scheme 14A.

| Compound | Structure | Yield |
|---|---|---|
| 1405 | 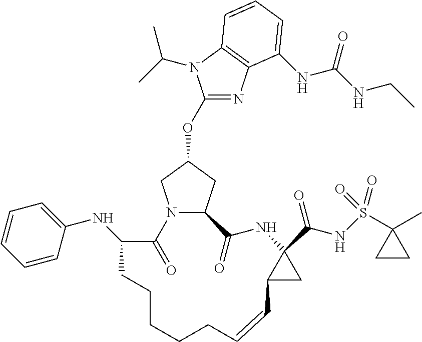 | 9.4 mg, yield 18%. MS (ESI) m/z (M + H)⁺ 803.2 |
| 1406 | 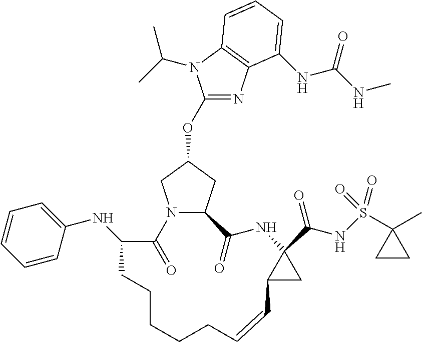 | 12.4 mg, yield 24%. MS (ESI) m/z (M + H)⁺ 789.2 |
| 1407 | 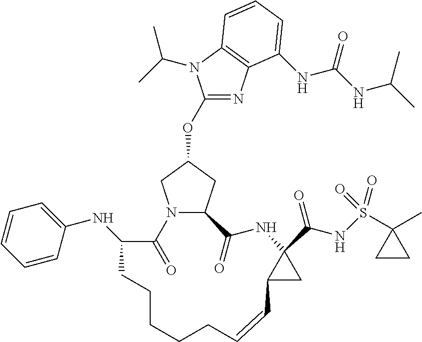 | 6.4 mg, yield 16%. MS (ESI) m/z (M + H)⁺ 817.4 |

### 14.6 Synthesis of Compounds 1408-1410

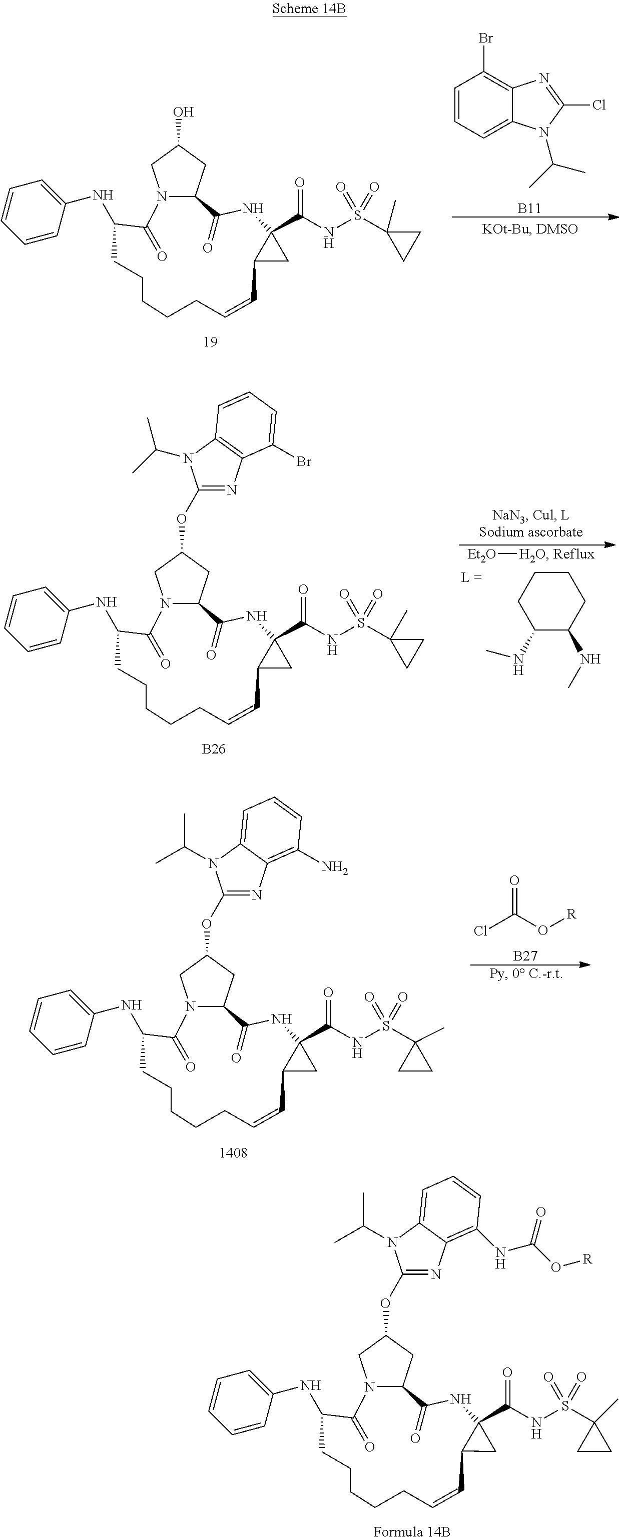

To a solution of compound 19 (200 mg, 0.36 mmol, 1 eq.) in 2 mL of DMSO was added KOt-Bu (1 mg, 1.75 mmol, 5 eq.) in portions at ambient temperature, then the mixture was stirred for 2 hrs at ambient temperature. After that, compound B11 (108 mg, 0.39 mmol, 1.1 eq.) was added, the resulting mixture was stirred at ambient temperature for 20 hrs, the reaction was monitored by LCMS. After completion of the reaction, the mixture was cooled by ice water, acidified by aq. HCl (2 M) to pH=6-7, extracted with EtOAc for three times. The organic layer was washed with brine, dried over anhydrous sodium sulfate, concentrated in vacuo to give crude product. It was purified with prep-TLC to give compound B26 (120 mg, yield 42%).

Compound B26 (160 mg, 0.20 mmol, 1 eq.), NaN3 (26 mg, 0.40 mmol, 2 eq.), ligand (14.2 mg, 0.1 mmol, 0.5 eq.), CuI (38 mg, 0.20 mmol, 1 eq.), sodium ascorbate (40 mg, 0.20 mmol, 1 eq.) and 2 mL of EtOH—H₂O (7:3) were introduced into a round-bottom flask equipped with a stirring bar and a reflux condenser. After it was degassed, and then introduced under nitrogen atmosphere, the reaction mixture was stirred under reflux for 8 hrs, the reaction was monitored by LCMS. After completion of the reaction, the mixture was cooled to room temperature, extracted by ethyl acetate (30 mL×3), the organic layers were combined, washed by brine, dried over anhydrous sodium sulfate, concentrated under reduced pressure, the residue was purified by prep-TLC to give compound 1408 (100 mg, yield 68%). MS (ESI) m/z (M+H)⁺ 732.3.

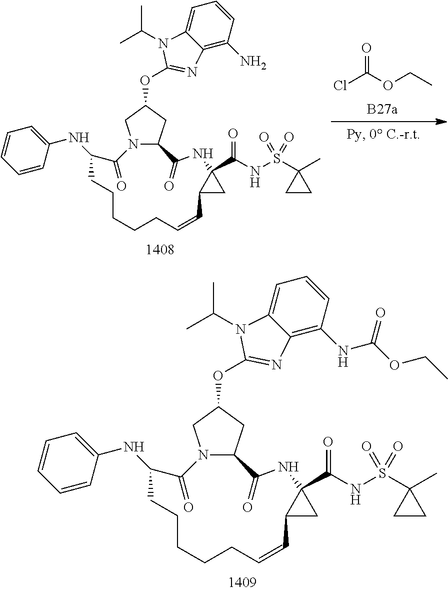

To a solution of compound 1408 (40 mg, 0.054 mmol, 1 eq.) in 2 mL of pyridine was added compound B27a (1.1 eq.) at 0° C. The solution was stirred for 2 hrs at 0° C., then allowed to warm to room temperature, and continued to stir for another 18 hrs. LCMS analysis showed the reaction completed. The reaction mixture was diluted with ethyl acetate, washed with aq. HCl (1 N), saturated aqueous NaHCO₃ and water. The combined organic layer was dried over anhydrous sodium sulfate, filtered. The solvent was removed under reduced pressure, the residue was purified by prep-TLC to give compound 1409 (15.1 mg, yield 34.4%). MS (ESI) m/z (M+H)⁺ 804.2.

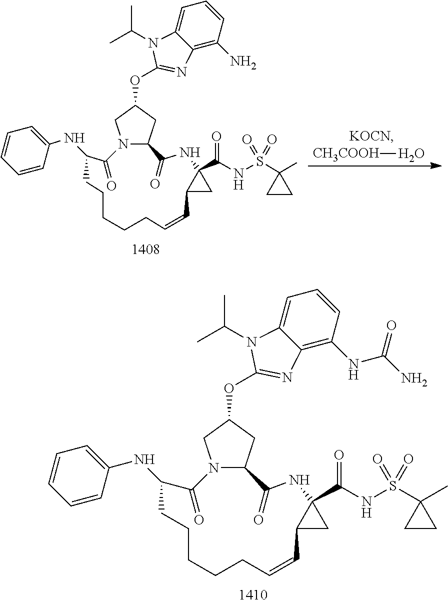

To a solution of 1408 (40 mg, 0.055 mmol, 1 eq.) in 1 mL of HOAc and 1.8 mL of H₂O was added KOCN (4.4 mg, 0.055 mmol, 1 eq.) in 1 mL of H₂O in portion during 30 min. After that, the reaction mixture was stirred for another 18 hrs at 30-40° C. LCMS showed the reaction completed. The mixture was diluted with water, extracted with ethyl acetate. The organic layer was washed with brine, dried over anhydrous sodium sulfate, concentrated under reduced pressure; the residue was purified by prep-TLC to give compound 1410 (16.3 mg, yield 39%). MS (ESI) m/z (M+H)⁺ 775.2.

### 14.7 Synthesis of Compounds 1411-1415

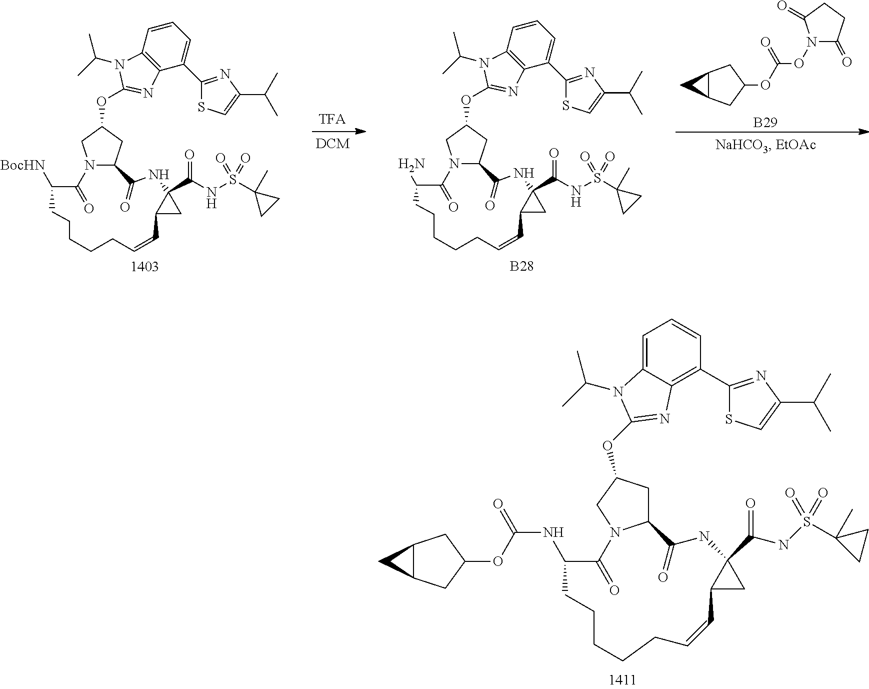

A flask was charged with compound 1403 (128 mg, 0.147 mmol), CF₃COOH (0.9 mL) and DCM (6 mL) was stirred at room temperature for overnight. The mixture was concentrated and diluted with EtOAc (50 mL), washed with saturated aq. NaHCO₃, dried over anhydrous sodium sulfate, concentrated under reduced pressure to give compound B28 (100 mg, 89%).

A flask was charged with B28 (80 mg, 0.091 mml), EtOAc (1 mL) and saturated aq. NaHCO₃ (1 mL). The reaction mixture was stirred at room temperature for one hour. Then a solution of compound B29 (30 mg, 0.091 mmol) in EtOAc (1 mL) was added and stirring continued for one hour at room temperature. The organic layer was separated and concentrated, purified by prep-HPLC to provide compound 1411 (40 mg, yield 50%). MS (ESI) m/z (M+H)⁺ 890.4.

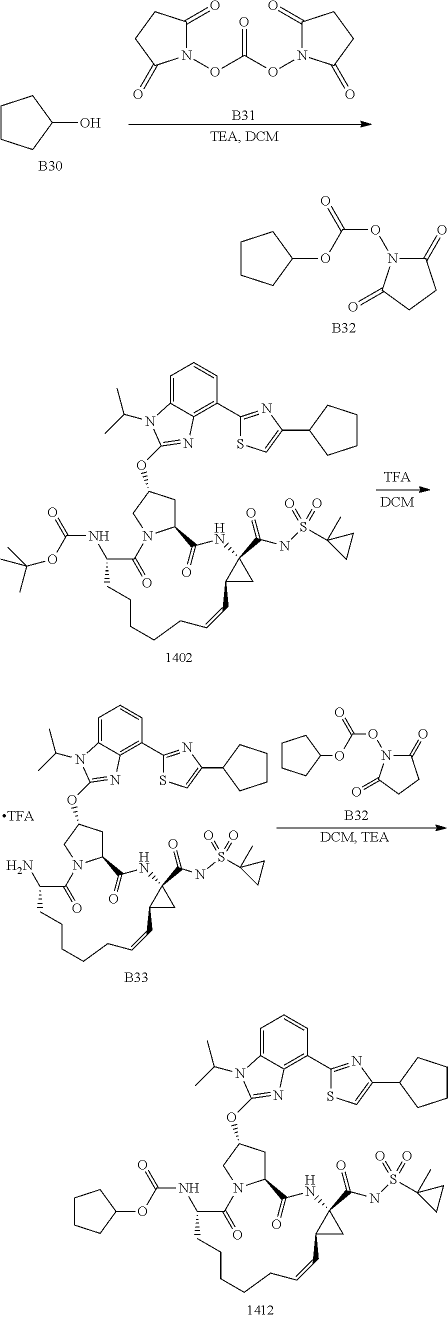

To a solution of compound B30 (870 mg, 10 mmol) in anhydrous DCM was added TEA (1.5 g, 15 mmol) and compound B31 (3.84 g, 15 mmol). The resulting mixture was stirred at room temperature for 2 days. The reaction was monitored by TLC. After completion of the reaction, the solvent was removed under reduced pressure. The residue was purified by column chromatography on silica gel to afford compound B32 (600 mg, yield 28%).

A flask was charged with compound 1402 (140 mg, 0.15 mmol), CF₃COOH (0.5 mL) and anhydrous DCM (3 mL). The resulting mixture was stirred at room temperature for 3 hrs. The reaction was monitored by LCMS. After completion of the reaction, the solvent was removed under reduced pressure to afford compound B33 (140 mg, yield 100%). The crude product was used directly in the next step without purification.

A flask was charged with compound B33 (80 mg, 0.1 mmol), TEA (0.05 mL, 0.4 mmol) and anhydrous DCM (4 mL). After stirred at room temperature for 30 min, compound B32 (43 mg, 0.2 mmol) was added. The resulting mixture was stirred at room temperature for 16 h. The reaction was monitored by LCMS. After completion of the reaction, the solvent was removed under reduced pressure. The residue was purified by prep-HPLC to afford compound 1412 (32.1 mg, yield 36%). MS (ESI) m/z (M+H)⁺ 904.4.

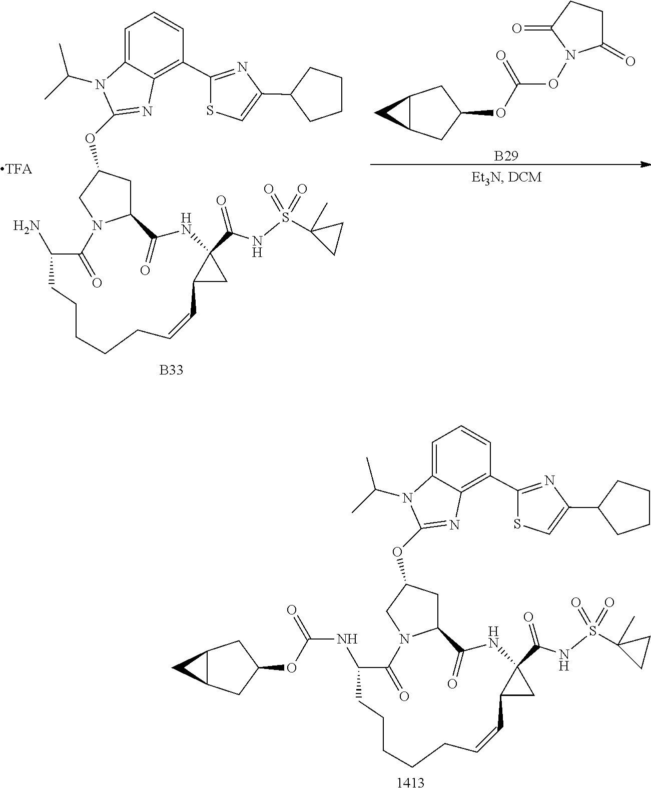

Compound B33 (133 mg, 0.15 mmol) and TEA (0.1 mL) was dissolved in DCM (4 mL), followed by the addition of compound B29 (54 mg, 0.225 mmol). The mixture was stirred at r.t. overnight. After completion of the reaction, the reaction mixture was diluted with DCM (20 mL), washed with water (10 mL) and brine (10 mL×2), dried over anhydrous sodium sulfate, concentrated under reduced pressure. The residue was purified by prep-HPLC to give compound 1413 (105.7 mg, yield 70%) as white solid. MS (ESI) m/z (M+H)⁺ 916.3.

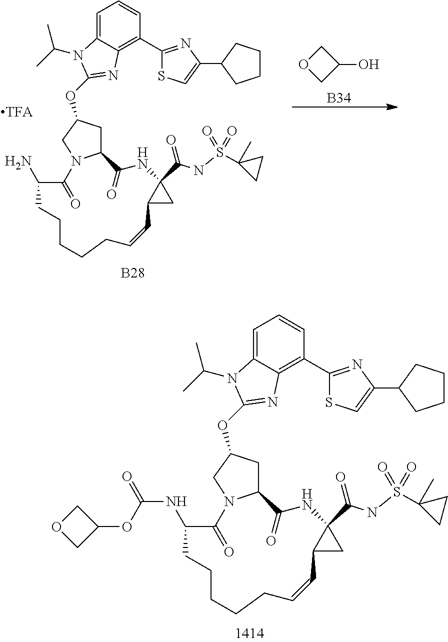

Compound B28 (121 mg, 1.64 mmol) and TEA (0.4 mL) was added into 8 mL of dry THF and stirred for 10 min. Then CDI (177 mg, 1.64 mmol) was added, the mixture was stirred at r.t. overnight. Compound B34 (130 mg, 0.164 mmol) was added into the above solution. The mixture was stirred at r.t. for 12 hrs. The reaction was quenched by water, the mixture was concentrated in vacuo. The resulting residue was purified by prep-TLC (DCM/MeOH=20:1) to provide compound 1414 (50 mg, yield 34%) as white-yellow solid. MS (ESI) m/z (M+H)⁺ 892.2.

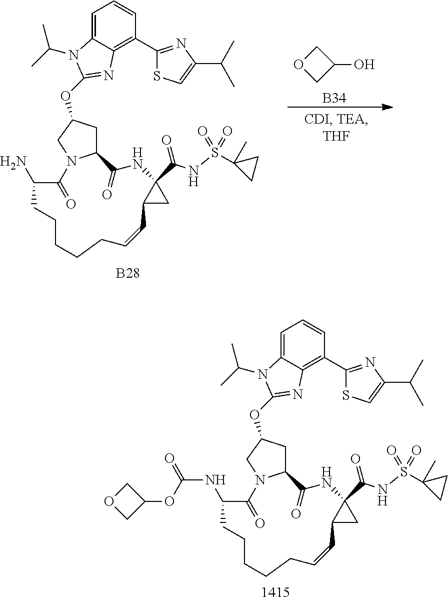

Compound B28 (170 mg, 0.231 mmol) and TEA (279 mg, 2.77 mmol) was added into 5 mL of anhydrous THF. The resulting mixture was stirred for 10 min. CDI (249 mg, 2.31 mmol) was added thereto, then the mixture was stirred at r.t. overnight. Then compound B34 (176 mg, 0.231 mmol) was added thereto. The mixture was stirred at r.t for 12 hrs. The reaction was quenched by water, the mixture was concentrated in vacuo. The resulting residue was purified by prep-TLC (DCM/MeOH=20:1) to give compound 1415 (102 mg, yield 51%). MS (ESI) m/z (M+H)⁺ 866.2.

### 14.7 Synthesis of Compounds 1416-1418

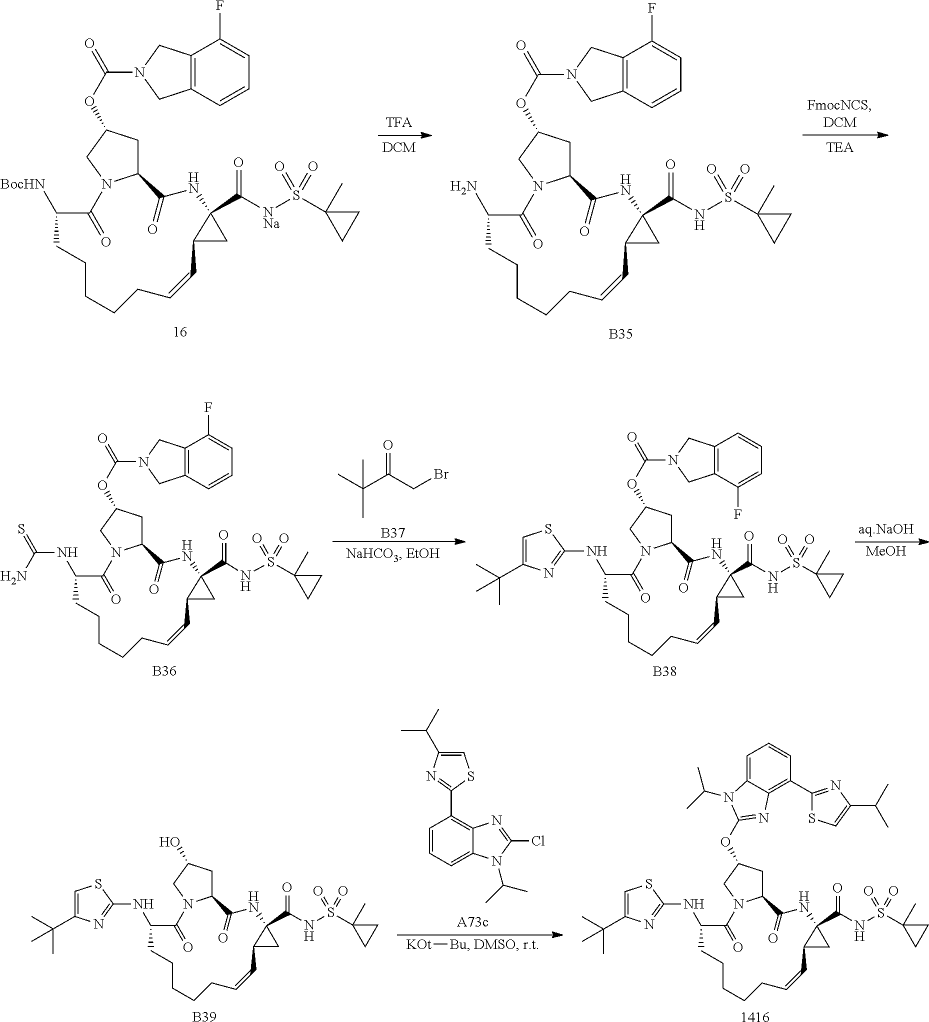

Compound B34 (1.5 g, 1.96 mmol) and TFA (3 mL) were added into 10 mL of DCM. The mixture was stirred at r.t for 2 hrs. TLC (PE/EA=1:3) showed compound B34 was consumed. The solution was made alkaline by addition of saturated aq. NaHCO₃, the organic layer was concentrated under reduced pressure to give crude compound B35 (890 mg, yield 71%).

Compound B35 (700 mg, 1.085 mmol), FmocNCS (365 mg, 1.3 mmol) and TEA (328 mg, 3.3 mmol) was added into 10 mL of DCM in turn. The mixture was stirred at r.t for 1 day and quenched by water. The mixture was adjusted to pH=7 by aq. HCl (1 M), extracted with EtOAc (20 mL×3). The organic layer was dried over anhydrous Na₂SO₄ and concentrated. The residue was dried in vacuo to give compound B36 as yellow solid (715 mg, yield 93%). MS (ESI) m/z [M+H]⁺ 705.

Compound B36 (500 mg, 0.71 mmol), Compound B37 (254 mg, 1.42 mmol) and NaHCO₃ (119 mg, 1.42 mmol) was added into 10 mL of EtOH in turn. The mixture was heated to reflux for 2 hrs, and quenched by water. The mixture was extracted with EtOAc (15 mL×3). The combined organic layers were washed with brine, dried over anhydrous Na₂SO₄ and concentrated in vacuo. The residue was purified by column chromatography on silica gel (PE/EA=8:1→6:1→4:1→1:1→1:2) to give compound B38 as pale-yellow solid (450 mg, yield 81%). MS (ESI) m/z [M+H]⁺ 785.

Compound B38 (410 mg, 0.52 mmol) and a solution of NaOH (624 mg, 15.6 mmol) in 2 mL of water was added into 10 mL of MeOH. The mixture was stirred at 40° C. for 4 days. The solution was concentrated to 4 mL and acidified to pH=5-6 with aq. HCl (1 M), then extracted with EtOAc (20 mL×3). The combined organic layer was washed with brine, dried over anhydrous Na₂SO₄ and concentrated in vacuo to provide crude compound B39 as white solid (306 mg, yield 95%). MS (ESI) m/z (M+H)⁺ 622.

To a solution of compound B39 (140 mg, 0.225 mmol) in DMSO (8 mL) was added KOt-Bu (106 mg, 0.945 mmol), the mixture was stirred for 1 h at r.t under nitrogen. After that, compound A73c (72 mg, 0.215 mmol) was added thereto, the reaction mixture was stirred at r.t for 12 hrs. After completion of the reaction, the reaction was quenched by ice water. The mixture was neutralized by aq. HCl (1 M), then extracted with EtOAc (20 mL×3). The combined organic layers was washed with brine, dried over anhydrous Na₂SO₄ and concentrated in vacuo. The residue was purified by prep-HPLC to give compound 1416 as white solid (31 mg, yield 15%). MS (ESI) m/z [M+H]⁺ 905.3.

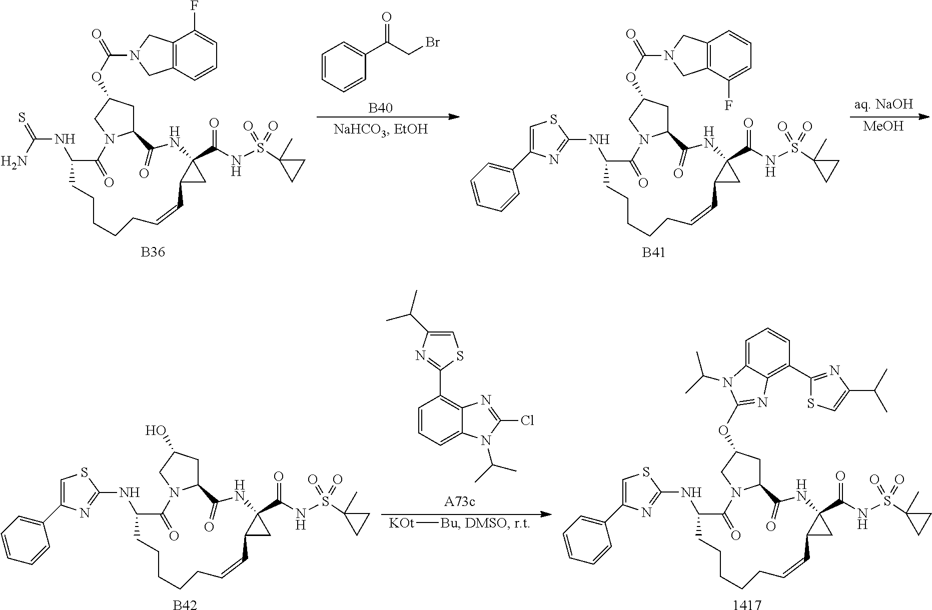

Compound B36 (500 mg, 0.71 mmol), Compound B40 (283 mg, 1.42 mmol) and NaHCO₃ (120 mg, 1.42 mmol) was added into 15 mL of EtOH in turn. The mixture was heated to reflux for 1.5 h, and quenched by water. The mixture was extracted with EtOAc (30 mL×x 3). The combined organic layers were washed with brine, dried over anhydrous Na₂SO₄ and concentrated in vacuo. The residue was purified by column chromatography on silica gel (PE/EA=8:1→6:1→4:1→1:1→1:2) to give compound B41 as a yellow solid (520 mg, yield 91%). MS (ESI) m/z [M+H]⁺ 805.

Compound B41 (320 mg, 0.4 mmol) and a solution of NaOH (477 mg, 12 mmol) in 2 nth of water was added into 15 mL of MeOH. The mixture was stirred at 40° C. for 4 days. The solution was concentrated to 4 mL and acidified to pH=5-6 with aq. HCl (1 M), then extracted with EtOAc (20 mL×3). The combined organic layer was washed with brine, dried over anhydrous Na₂SO₄ and concentrated in vacuo to provide crude compound B42 as yellow solid (231 mg, yield 92%). MS (ESI) m/z (M+H)⁺ 642.2.

Compound 1417 was prepared using a procedure that is similar to that of preparation of compound 1416 (65 mg, yield 38%). MS (ESI) m/z [M+H]⁺ 925.2.

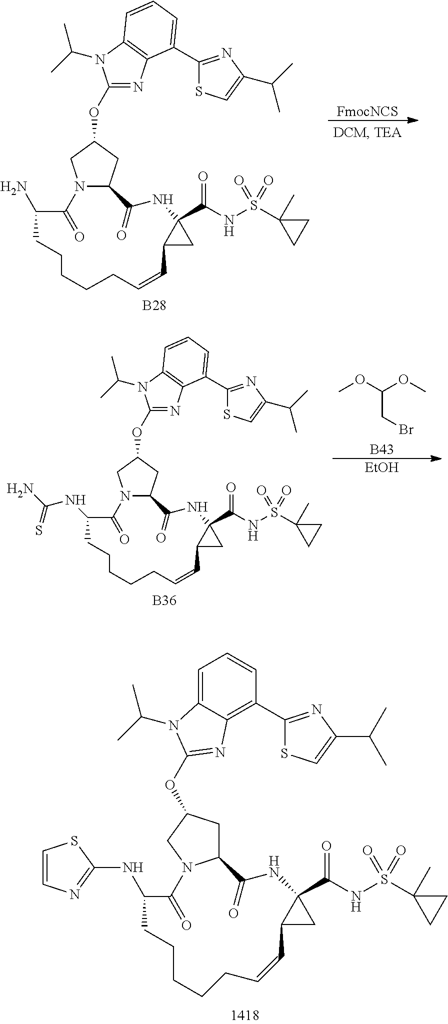

Compound B28 (370 mg, 0.48 mmol), FmocNCS (202 mg, 0.72 mmol) and TEA (2.5 mL) was added into 10 mL of DCM in turn at 0° C. After stirred for 15 min at 0° C., the ice bath was removed and the mixture was stirred at r.t. overnight. The reaction was monitored by LCMS. After completion of the reaction, the mixture was neutralized to pH=6˜7 with aq. HCl (0.1M), and then diluted with EtOAc (100 mL). Organic phase was separated, washed with brine, dried over anhydrous Na₂SO₄ and concentrated under reduced pressure to give a crude product, which was purified by prep-TLC to afford compound B36 (300 mg, yield 76%).

To a solution of compound B36 (30 mg, 0.036 mmol) in EtOH (3 ml) was added compound B43 (30.4 mg, 0.18 mmol). The mixture was heated to reflux under nitrogen. The reaction was monitored by LCMS. After completion of the reaction, the mixture was concentrated under reduced pressure to give a crude product, which was purified by prep-HPLC to provide compound 1418 (18 mg, yield 60%). MS (ESI) m/z [M+H]⁺ 849.3.

### 14.8 Synthesis of Compound 1419-1426

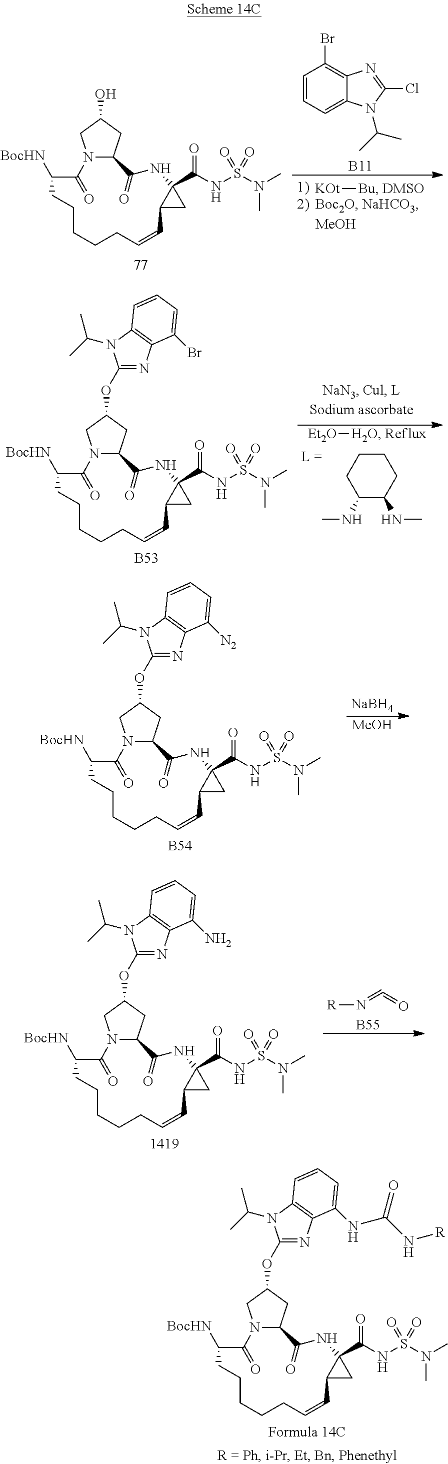

To a solution of compound 77 (200 mg, 0.35 mmol, 1 eq.) in 2 mL of DMSO was added t-BuOK (196 mg, 1.75 mmol, 5 eq.) in portions at ambient temperature, then the mixture was stirred for 2 h at ambient temperature. After that, compound B11 (105 mg, 0.38 mmol, 1.1 eq.) was added, the resulting mixture was stirred at ambient temperature for 20 h, the reaction was monitored by LCMS. After completion of the reaction, the mixture was cooled by ice water, acidified by aq. HCl (2 M) to pH=7-8. Then NaHCO₃ (35.6 mg, 0.42 mmol, 1.2 eq.) were added. The mixture was stirred another 17 h, extracted by ethyl acetate (50 mL×3), the organic layers were combined, washed by brine, dried over anhydrous sodium sulfate, concentrated under reduced pressure, the residue was purified by prep-TLC to give compound B53 (130 mg, 57.5%).

Compound B53 (130 mg., 0.16 mmol, 1 eq), NaN₃ (21 mg, 0.32 mmol, 2 eq.), ligand (3.4 mg, 0.024 mmol, 0.15 eq.), CuI (31 mg, 0.16 mmol, 1 eq.), sodium ascorbate (32 mg, 0.16 mmol, 1 eq.) and 2 mL of EtOH—H₂O (7:3) were introduced into a round-bottom flask equipped with a stirring bar and a reflux condenser. After it was degassed, and then introduced under nitrogen atmosphere, the reaction mixture was stirred under reflux for 8 h, the reaction was monitored by LCMS. After completion of the reaction, the mixture was cooled to room temperature, extracted by ethyl acetate (30 mL×3), the organic layers were combined, washed by brine, dried over anhydrous sodium sulfate, concentrated under reduced pressure, the residue was purified by prep-TLC to give compound B54 (80 mg, 66.7%).

To a solution of compound B54 (35 mg, 0.045 mmol, 1 eq.) in 3 mL of methanol was added NaBH₄ (51 mg, 1.36 mmol, 30 eq.). The solution was stirred at room temperature. TLC analysis showed the reaction completed. All the volatiles was removed under reduced pressure. The residual was diluted with water, extracted with ethyl acetate (30 mL×3), the organic layers were combined, washed by brine, dried over anhydrous sodium sulfate, concentrated under reduced pressure, the residue was purified by prep-TLC to give compound 1419 (20 mg, 60.6%).

To a solution of compound 1419 (1 eq.) in 1 mL of DMAC or THF was added compound B55 (1.1 eq.). The mixture was heated to 60° C. for 20 h. TLC analysis showed the reaction completed. The reaction mixture was diluted with water, extracted with ethyl acetate (30 mL×3), the organic layers were combined, washed by brine, dried over anhydrous sodium sulfate. The solvent was removed under reduced pressure; the residue was purified by prep-TLC to give compound of Formula 14C. The following compounds were prepared according to this step.

TABLE 15
Compounds 1420-1424.

| Compound | Structure | Yield |
|---|---|---|
| 1420 | 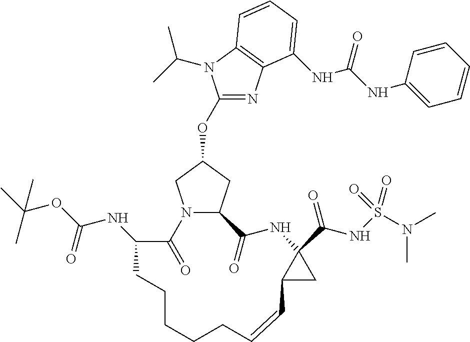 | 13.4 mg, 29.2%. MS (ESI) m/z (M + H)⁺ 864.4. |
| 1421 | 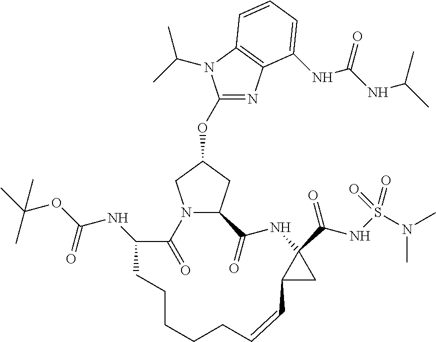 | 20.0 mg, 44.9%. MS (ESI) m/z (M + H)⁺ 830.3. |
| 1422 | 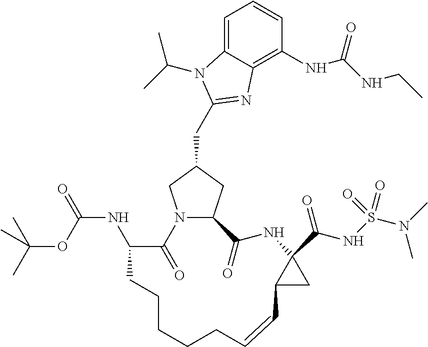 | 8.8 mg, 44.9%. MS (ESI) m/z (M + H)⁺ 815.8. |
| 1423 | 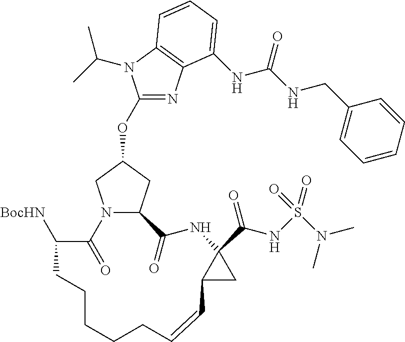 | 15.6 mg, 44.6%. MS (ESI) m/z (M + H)⁺ 878.3. |
| 1424 | 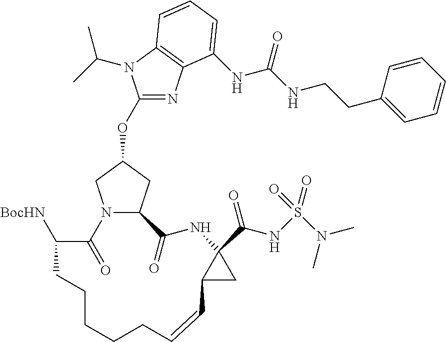 | 25 mg, 70.2%. MS (ESI) m/z (M + H)⁺ 891.6. |

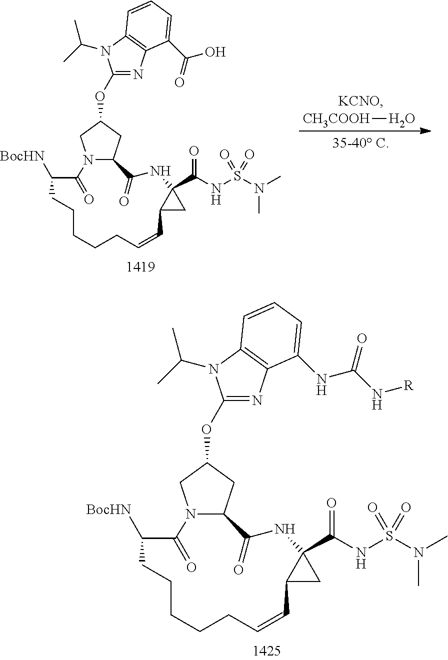

To a solution of compound 1419 (40 mg, 1 eq.) in 1 mL of acetic acid and 1.8 mL of H₂O was added KCNO (1 eq.) in 1 mL of H₂O in portion during 30 min. After that, the reaction mixture was stirred for another 18 h at 30-40° C. LCMS showed the reaction completed. The mixture was diluted with water, extracted with ethyl acetate. The organic layer was washed with brine, dried over anhydrous sodium sulfate, concentrated under reduced pressure, the residue was purified by prep-TLC to give compound 1425, 16.3 mg, 38.8%, MS (ESI) m/z (M+H)⁺ 788.3.

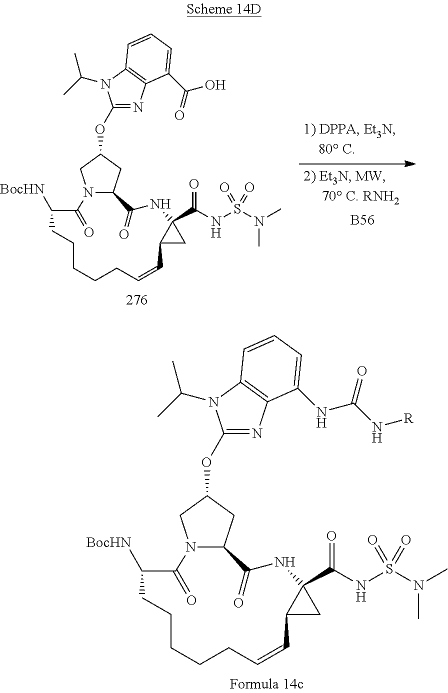

General procedure: To a solution of the macrocyclic intermediate 276 in anhydrous toluene was added TEA (5 eq.) and DPPA (3 eq.), the resulting mixture was stirred at 80° C. under nitrogen atmosphere. The reaction was detected by LCMS. After the reaction complete, the mixture was poured into a microwave tube, then amine B56 (3 eq.) was added and sealed. The mixture was heated at 70° C. by microwave for 20 min. Then the reaction mixture was diluted with ethyl acetate and washed with brine. The organic phase was gathered, dried over anhydrous sodium sulfate and concentrated in vacuo. The residue was purified by prep-HPLC to give compound of Formula 14C.

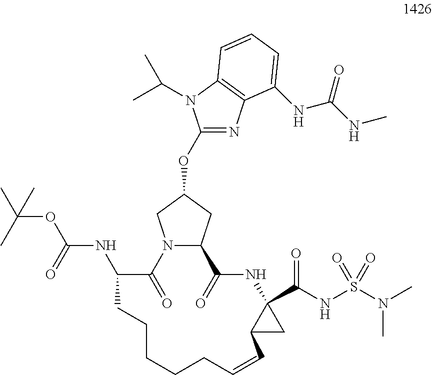

Compound 1426 (5.7 mg, 20.6%. MS (ESI) m/z (M+H)⁺ 802.3) was made according to Scheme 14D.

### 14.9 Synthesis of Compounds 1427-1429

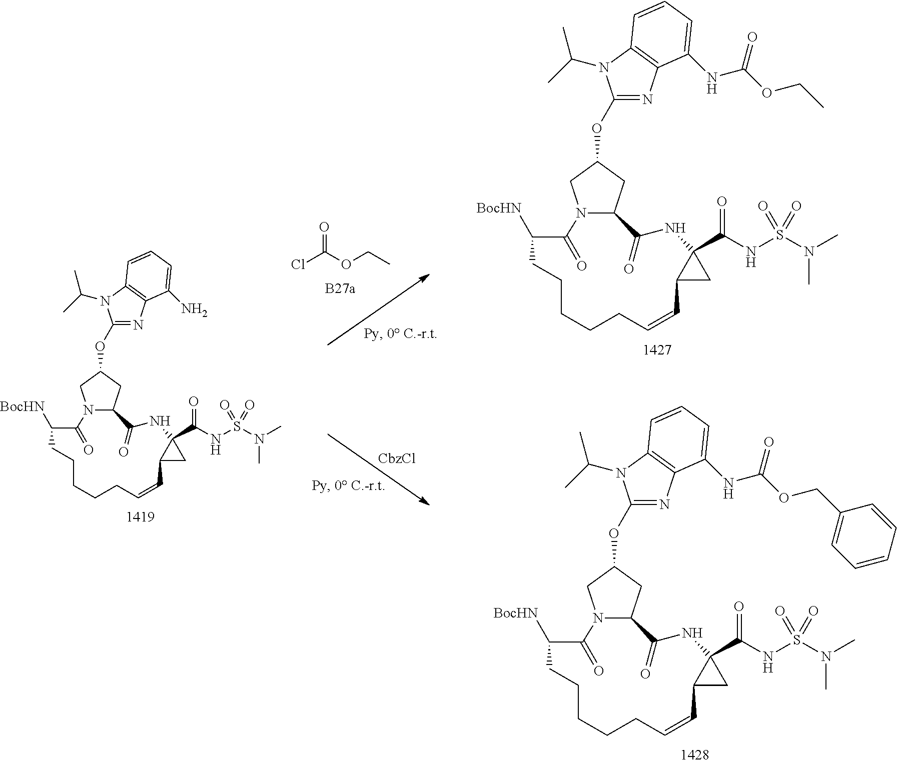

To a solution of compound 1419 (40 mg, 0.054 mmol, 1 eq.) in 2 mL of pyridine was added compound B27a (1.1 eq.) at 0° C. The solution was stirred for 2 hrs at 0° C., then allowed to warm to room temperature, and continued to stir for 18 hrs. LCMS analysis showed the reaction completed. The reaction mixture was diluted with ethyl acetate, washed with aq. HCl (1 M), saturated aqueous NaHCO₃ and water. The combined organic layer was dried over anhydrous sodium sulfate, filtered. The solvent was removed under reduced pressure, the residue was purified by prep-TLC to provide compound 1427 (7.7 mg, 17.5%). MS (ESI) m/z (M+H)⁺ 817.3. Compound 1428 (18.4 mg, 40.0%. MS (ESI) m/z (M+H)⁺ 879.3) was also prepared according to the same procedure.

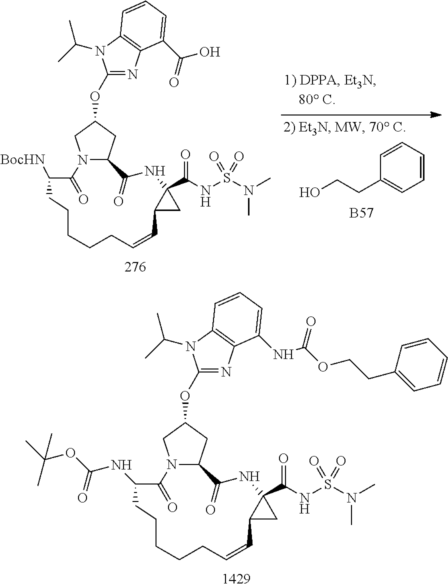

To a solution of the macrocyclic intermediate 276 in anhydrous toluene was added TEA (5 eq.) and DPPA (3 eq.), the resulting mixture was stirred at 80° C. under nitrogen atmosphere. The reaction was detected by LCMS. After the reaction complete, the mixture was poured into a microwave tube, then compound B57 (3 eq.) was added and the tube was sealed. The mixture was heated at 70° C. by microwave for 20 min. Then the reaction mixture was diluted with ethyl acetate and washed with brine. The organic phase was gathered, dried over anhydrous sodium sulfate and concentrated in vacuo. The residue was purified by prep-HPLC to give compound 1429. 6.9 mg, yield 15%. MS (ESI) m/z (M+H)⁺ 894.0.

### 14.10 Synthesis of Compounds 1430-1436

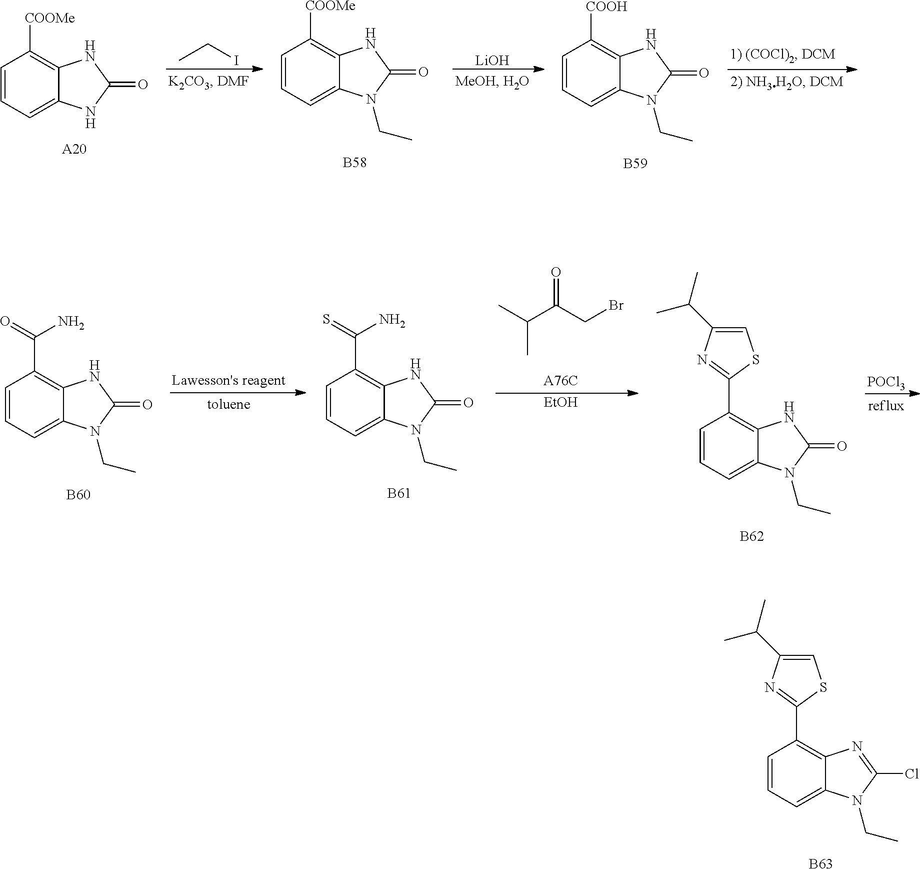

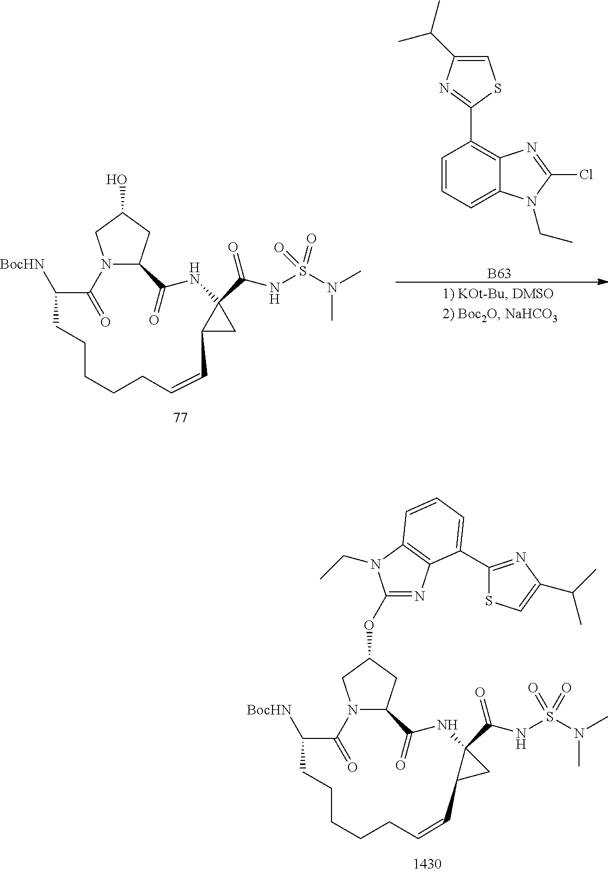

To a solution of compound A20 (4.6 g, 23.9 mmol) and K₂CO₃ (6.6 g, 47.9 mmol) in anhydrous DMF (100 mL) was added iodoethane (4.1 g, 26.3 mmol). The mixture was stirred for 12 hrs at r.t. under nitrogen. After completion of the reaction, the mixture was neutralized with aq. HCl (2 M), and then extracted with EtOAc (50 mL×3). The combined organic layer was washed with brine, dried over anhydrous Na₂SO₄ and concentrated to give a crude product, which was purified by column chromatography on silica gel (eluent PE/EA=10:1˜3:1) to yield compound B58 (2.7 g, yield 51%). MS (ESI) m/z (M+H)⁺ 220.8.

To a solution of compound B58 (2.7 g, 12.3 mmol) in MeOH (40 mL) and water (20 mL) was added LiOH (3.0 g, 123 mmol). The mixture was stirred for 12 hrs at r.t. After that, the reaction mixture was acidified to pH=3 with aq. HCl (2 M) and then extracted with EtOAc. The combined organic layer was washed with brine, dried over anhydrous Na₂SO₄ and concentrated to give a crude product B59, which was used directly in next step (2.5 g, yield 98%). MS (ESI) m/z (M+H)⁺ 206.8. ¹H NMR (300MHz, DMSO-d₆) δ: 10.6 (s, 1H), 7.45 (d, J=8.1 Hz, 1H), 7.33 (d, J=7.8 Hz, 1H), 7.04 (t, J=7.8 Hz, 1H), 3.85-3.78 (m, 2H), 1.15 (t, J=7.2 Hz, 3H).

To a solution of compound B59 (2.5 g, 12.1 mmol) in anhydrous DCM (60 mL) was added oxalyl chloride (4.6 g, 36.4 mmol) at 0° C., and followed by DMF (two drops) at 0° C. The mixture was stirred for 15 min at 0° C. and then stirred for 15 min at r.t. After completion of the reaction, the solvent was evaporated under reduced pressure to give a crude acyl chloride. To a solution of the acyl chloride in anhydrous DCM (80 mL) was added ammonia (9.7 mL), and then the mixture was stirred for 12 hrs at r.t. After that, solids were filtered off, washed with DCM, and dried over vacuum freeze-drier to give a white solid compound B60, which was used directly in next step (2.0 g, yield 81%). MS (ESI) m/z (M+H)⁺ 205.9.

A flask was charged with compound B60 (2.0 g, 9.8 mmol), Lawesson's reagent (2.0 g, 4.9 mmol) and anhydrous toluene (60 mL). The mixture was refluxed under nitrogen. After completion of the reaction, solids were filtered off and washed with EtOAc to provide a yellow crude product compound B61, which was used directly in next step (1.6 g, yield 72%).

To a solution of compound B61 (345 mg, 1.56 mmol) in EtOH (5 mL) was added compound A76c (514 mg, 3.12 mmol). The mixture was refluxed under nitrogen. After completion of the reaction, the solvent was evaporated under reduced pressure to give a crude product, which was isolated by prep-TLC (PE/EA=3:1) to yield compound B62 (201 mg, yield 45%). MS (ESI) m/z (M+H)⁺ 287.8.

Compound B62 (201 mg, 0.70 mmol) was dissolved in POCl₃ (3 mL) and then the mixture was refluxed under nitrogen. After completion of reaction, most of POCl₃ was evaporated, and then the mixture was taken up with ice-water, neutralized with ammonia under cooling, then extracted with EtOAc (20 mL×3). The combined organic layer was washed with brine, dried over anhydrous Na₂SO₄ and concentrated to give a crude product (compound B63), which was used directly in next step (168 mg, yield 79%).

Compound 77 (314 mg, 0.55 mmol) was dissolved in DMSO (4 mL) and the solution was degassed with nitrogen. Then KOt-Bu (278 mg, 2.48 mmol) was added thereto and the mixture was stirred for 1 h at r.t. under nitrogen. After that, compound B63 (168 mg, 0.55 mmol) was added and then the mixture was stirred for 12 hrs at r.t. under nitrogen. The reaction was monitored by LCMS. The De-Boc product was detected. The reaction was quenched with ice-water. The mixture was acidified to pH=6-7 with aq. HCl (0.1 M) and then MeOH (5 mL), NaHCO₃ (55 mg, 0.66 mmol), Boc₂O (120 mg, 0.55 mmol) were added. The mixture was stirred for 1 h at r.t. After that, MeOH was evaporated under reduced pressure and the resulting mixture was acidified to pH=5-6 with aq. HCl (0.1 M), then extracted with EtOAc (30 mL×3). The combined organic layer was washed with brine, dried over anhydrous Na₂SO₄ and concentrated to give a crude product, which was purified by prep-HPLC to afford compound 1430 (73.4 mg, yield 17%). MS (ESI) m/z (M+H)⁺841.2.

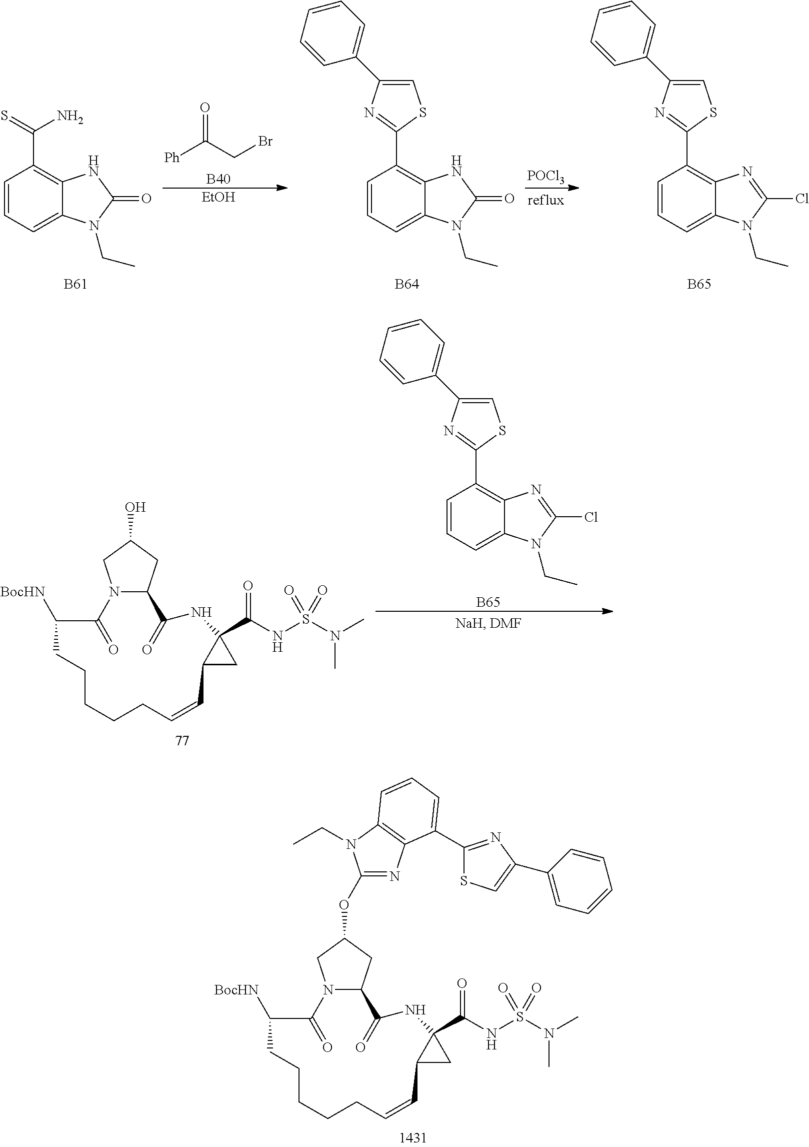

To a solution of compound B60 (150 mg, 0.68 mmol) in EtOH (3 mL) was added compound B40 (162 mg, 0.81 mmol). The mixture was refluxed under nitrogen. After completion of the reaction, the solvent was evaporated under reduced pressure to give a crude product, which was purified by prep-TLC (PE/EA=3:1) to afford compound B64 (128 mg, yield 59%). MS (ESI) m/z (M+H)⁺ 321.8.

Compound B64 (128 mg, 0.40 mmol) was dissolved in POCl₃ (3 mL) and then the mixture was refluxed under nitrogen. After completion of reaction, most of POCl₃ was evaporated, and then the mixture was taken up with ice-water, neutralized with ammonia under cooling, then extracted with EtOAc (20 mL×3). The combined organic layer was washed with brine, dried over anhydrous Na₂SO₄ and concentrated to give a crude product compound B65, which was used directly in next step (123 mg, yield 91%).

Compound 77 (206 mg, 0.36 mmol) was dissolved in DMF (2 mL) and the solution was degassed with nitrogen. Then the solution was cooled to 0° C. and NaH (60% dispersion in mineral oil, 115 mg, 2.88 mmol) was added. The mixture was stirred for 1 h at 0° C. under nitrogen. Then compound B65 (123 mg, 0.36 mmol) was added and then the mixture was stirred for 12 hrs at r.t. under nitrogen. The reaction was monitored by LCMS. After completion of reaction, the reaction was quenched with ice-water, and neutralized with aq. HCl (0.1 M), extracted with EtOAc (20 mL×3). The combined organic layer was washed with brine, dried over anhydrous Na₂SO₄, and concentrated in vacuo to yield a crude product, which was purified by prep-HPLC to provide compound 1431 (75.5 mg, yield 24%). MS (ESI) m/z (M+H)⁺ 875.3.

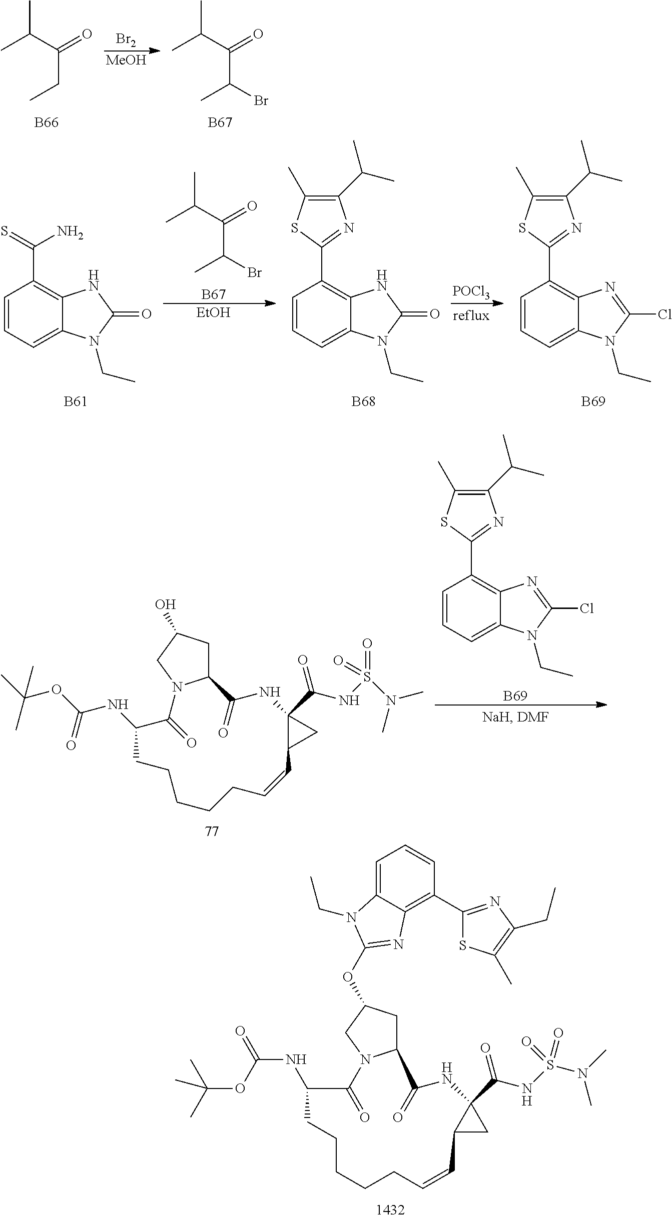

To a solution of compound B66 (15 g, 150 mmol) in MeOH (150 mL) was added Br₂ (9 mL, 180 mmol) in portions at 0° C. The mixture was stirred at room temperature for 1.5 h and added with water (150 ml), then stirred for another 15 min. The mixture was concentrated and extracted with EtOAc. The organic layer was washed successively with saturated NaHCO₃ and brine, dried over anhydrous Na₂SO₄, and concentrated to give crude compound B67 (10 g), which was used directly in the next step.

A flask was charged with compound B61 (44 mg, 0.199 mmol), B67 (0.1 mL, crude) and EtOH (2 mL). The mixture was stirred at 110° C. for 2 hrs. After completion of reaction, the mixture was concentrated and purified by prep-TLC to yield compound B68 (20 mg, yield 33%). ¹H NMR (CDCl₃) δ: 9.64 (s, 1H), 7.26 (t, J=13.5 Hz, 1H), 7.06 (s, 1H), 6.95 (t, J=7.6 Hz, 1H), 3.96-3.93 (m, 2H), 3.11-3.06 (m, 1H), 2.40 (s, 1H), 1.36-1.29 (m, 9H).

Compound B68 (20 mg, 0.06 mmol) was dissolved in POCl₃ (2 mL) and then the mixture was refluxed for 4 hrs under nitrogen. After completion of reaction, most of POCl₃ was evaporated, and then the mixture was taken up with ice-water, neutralized with ammonia under cooling, then extracted with EtOAc (20 mL×3). The combined organic layer was washed with brine, dried over anhydrous Na₂SO₄ and concentrated to give a crude product compound B69, which was used directly in next step (20 mg, yield 94%).

The procedure for preparation of compound 1432 is similar to that of preparation of compound 1431. 7 mg, yield 12%. MS (ESI) m/z (M+H)⁺ 855.4.

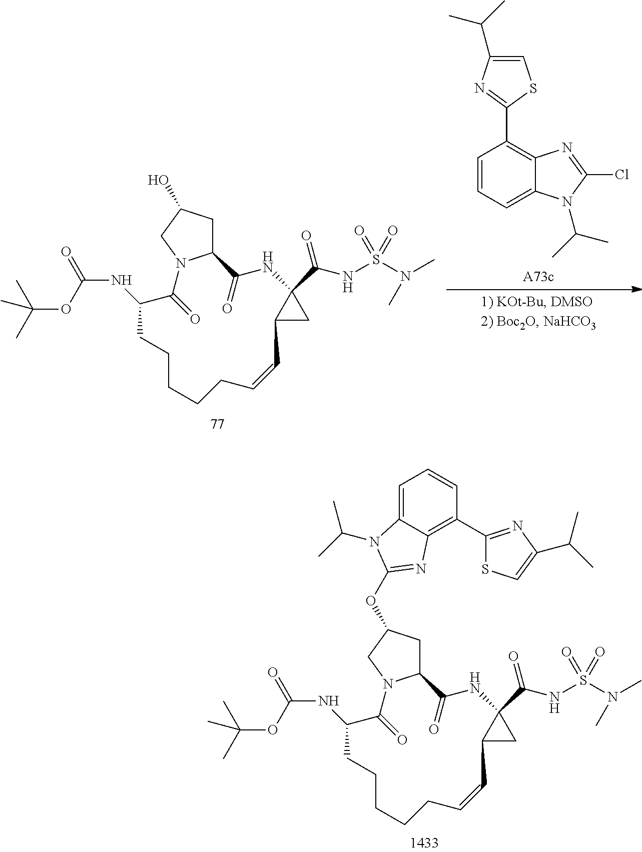

The procedure for preparation of compound 1433 is similar to that of preparation of compound 1431. 230 mg, yield 51%. MS (ESI) m/z (M+H)⁺ 855.3.

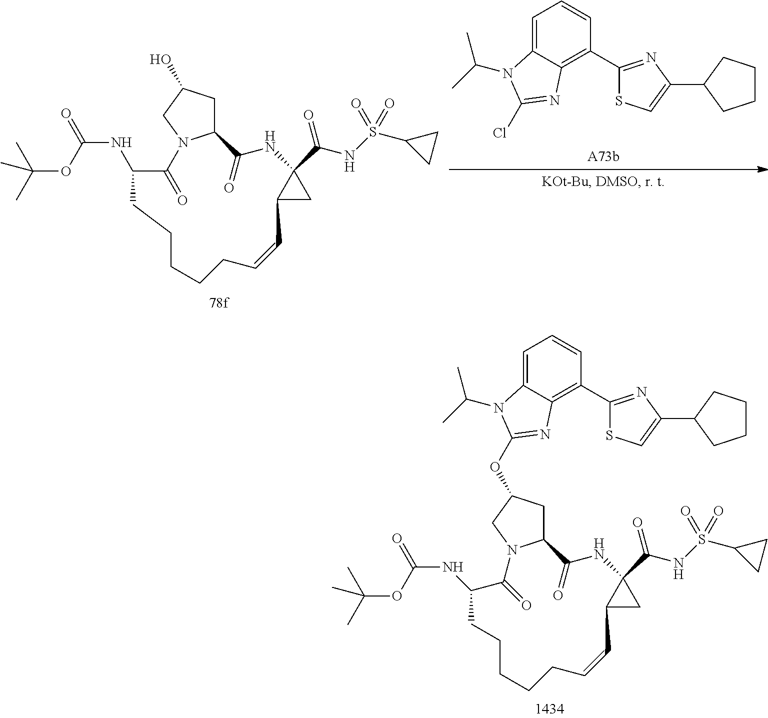

The procedure for preparation of compound 1434 is similar to that of preparation of compound 1431. 73.6 mg, yield 24%. MS (ESI) m/z (M+H)⁺ 878.2.

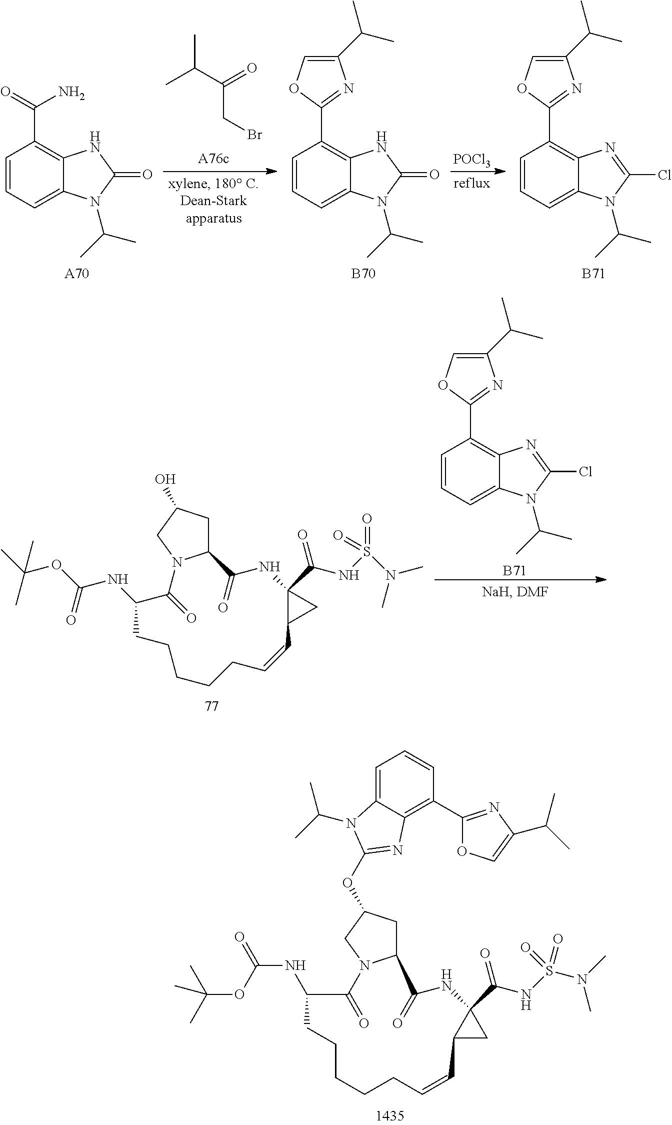

A flask was charged with compound A70 (890 mg, 4 mmol), compound A76c (1.34 g, 8 mmol) and xylene. The mixture was stirred at 130° C. with Dean-Stark apparatus to remove water. After stirring for 36 hrs, the mixture was concentrated and purified by prep-TLC to afford compound B70 (200 mg, yield 17.5%). ¹H NMR (CDCl₃) δ: 9.50 (s, 1H), 7.50 (t, J=7.6 Hz, 1H), 7.31 (s, 1H), 7.11 (t, J=7.6 Hz, 1H), 7.06-7.02 (m, 1H), 4.73-4.66 (m, 1H), 2.85-2.80 (m, 1H), 1.49 (d, J=6.8 Hz, 6H), 1.22 (m, 6H).

A flask (10 mL) was charged with compound B70 (200 mg, 0.7 mmol) and POCl₃ (4 mL), and then the mixture was refluxed for 4 hrs under nitrogen. After completion of reaction, most of POCl₃ was evaporated, the residue was diluted with EtOAc (50 mL), washed by saturated aq. NaHCO₃, water and dried over anhydrous Na₂SO₄ and concentrated to yield compound B71 (170 mg, yield 80%).

The procedure for preparation of compound 1435 is similar to that of preparation of compound 1431. 35 mg, yield 10%. MS (ESI) m/z (M+H)⁺ 839.4.

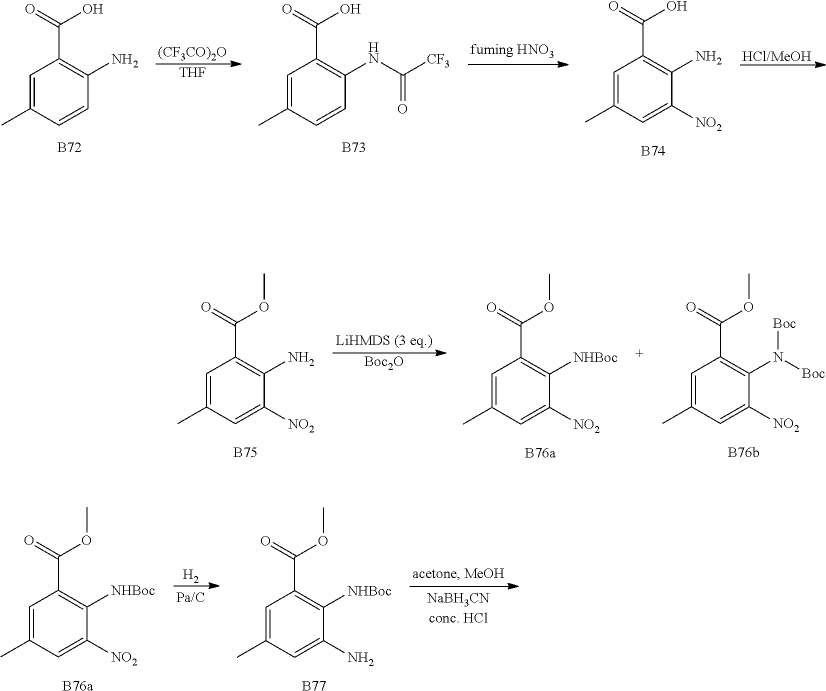

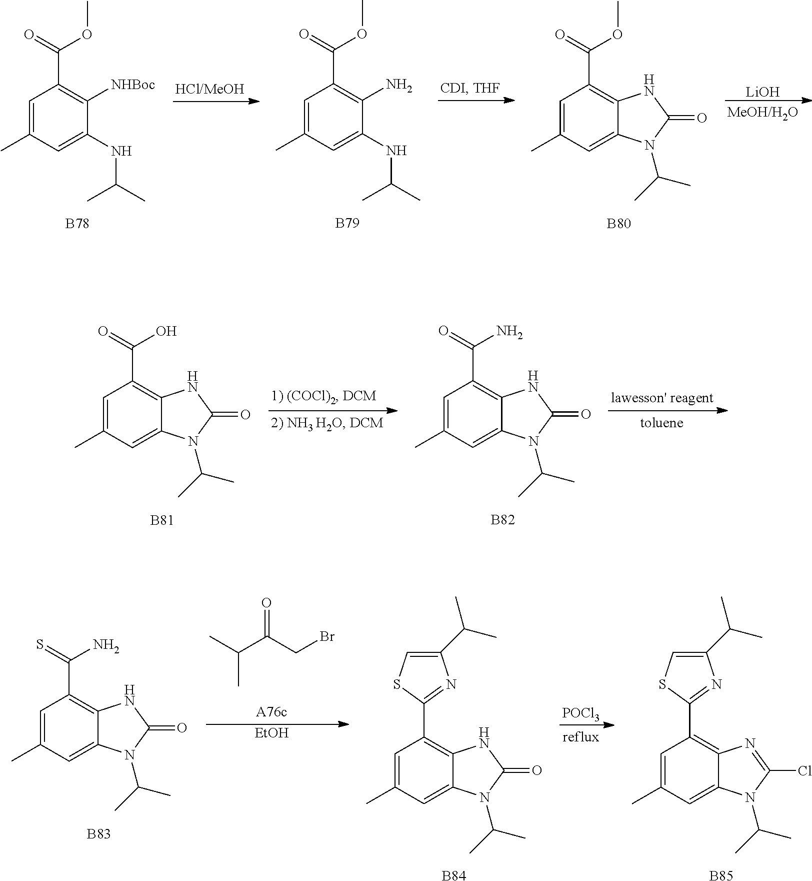

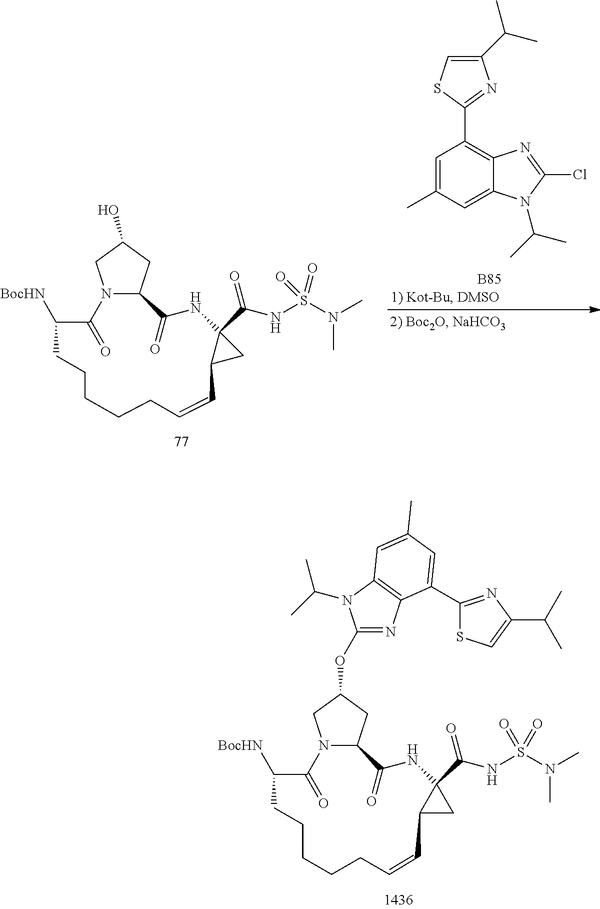

To a solution of compound B72 (5 g, 33 mmol) in anhydrous THF (25 mL) was added dropwise (CF₃CO)₂O (25 mL) at 0° C. The resulting solution was allowed to stir at 30° C. for 16 hrs. Then the solution was poured into ice-water. The solid was filtered, washed with water and dried in vacuum to provide compound B73 (5.6 g, yield 69%).

To a fuming nitric acid (20 mL) was added compound B73 (5.6 g, 22.7 mmol) in one portion at 4° C. The resulting solution was allowed to stir at 4° C. for 1 h. The solution was poured into ice-water, filtered, washed with water and dried in vacuum to afford crude compound B74 (5.0 g, crude yield 113%).

A flask was charged with compound B74 (5.0 g, 25.5 mmol) and HCl/MeOH (4 M, 50 mL). The resulting mixture was heated to reflux and maintained this temperature for 16 hrs. The reaction was monitored by TLC. After completion of the reaction, the solvent was removed under reduced pressure. The residue was neutralized with TEA, then, diluted with EtOAc (200 mL), washed with brine, dried over anhydrous Na₂SO₄ and concentrated under reduced pressure to afford compound B75 (3.0 g, yield 56%).

To a solution of compound B75 (2.0 g, 9.5 mmol) in anhydrous THF (30 mL) at 0° C. was added Boc₂O (8.3 g, 38 mmol) in one portion and then LiHMDS (1.0 M solution in THF, 28.5 mL, 28.5 mmol) was added dropwise via syringe over 15 min. The resulting solution was stirred at room temperature for 3.5 hrs. Then the reaction was quenched by saturated aq. NH₄Cl. The solvent was evaporated and the mixture was extracted with EtOAc (50 mL×3). The combined organic layer was washed with brine, dried over anhydrous sodium sulfate, concentrated under reduced pressure. The crude product was purified by column chromatography on silica gel to afford compound B76a (380 mg, yield 13%) and compound B76b (1.6 g, yield 41%).

To a solution of compound B76a (380 mg, 1.23 mmol) in MeOH (20 mL) was added Pd/C (0.2 g). The resulting mixture was then stirred at room temperature under H₂ (15 Psi) for 4 hrs. The reaction was monitored by TLC. After completion of the reaction, the catalyst was filtered and the solvent was removed under reduced pressure to afford compound B77 (340 mg, yield 98.8%).

To a solution of compound B77 (340 mg, 1.2 mmol) in MeOH (20 mL) was added acetone (0.17 mL, 2.4 mmol) and conc. HCl (0.13 mL). The resulting mixture was stirred at room temperature for 30 min. Then NaBH₃CN (113 mg, 1.8 mmol) was added. The reaction was monitored by TLC. After completion of the reaction, the solvent was removed under reduced pressure to afford crude compound B78 (380 mg, crude yield 98.4%).

A flask was charged with compound B78 (380 mg, 1.18 mmol) and HCl/MeOH (20 mL) under nitrogen at room temperature stirred for 30 min. The reaction was monitored by TLC. After completion of the reaction, the solvent was removed under reduced pressure. The residue was neutralized by saturated aq. NaHCO₃, extracted with EtOAc (50 mL×3), the organic layer was combined and washed with brine, dried over anhydrous sodium sulfate, the solvent was removed under reduced pressure to provide crude compound B79 (280 mg, crude yield 106.8%).

A microwave tube was charged with compound B79 (280 mg, 1.26 mmol), CDI (822 mg, 5.0 mmol) and anhydrous THF (20 mL). The reaction vessel was sealed and heated in microwave at 120° C. for 1 hour. The reaction was monitored by TLC. After completion of the reaction, the reaction mixture was acidified by aq. HCl (0.1 M). Then most of the solvent was removed under reduced pressure. The solid was filtered, washed with aq. HCl (0.1 M) and collected to afford compound B80 (210 mg, yield 71%).

A flask was charged with compound B80 (210 mg, 0.9 mmol), LiOH (216 mg, 9 mmol), MeOH (30 mL) and H₂O (30 mL). The mixture was stirred for 16 h at 40° C. The reaction was monitored by TLC. After completion of the reaction, most of the solvent was removed under reduced pressure, the residue was acidified by aq. HCl (0.1 M). A precipitate was formed. The solid was collected by filtration and washed by water to afford compound B81 (140 mg, yield 70%).

To a solution of compound B81 (140 mg, 0.6 mmol) in anhydrous DCM (10 mL) was added oxalyl chloride (0.1 mL, 1.2 mmol), followed by a drop of DMF. The resulting suspension was stirred at room temperature until all solid was dissolved, then the solvent was removed under reduced pressure. The residue was dissolved in anhydrous DCM (10 mL) again, ammonia was added thereto. Solid was precipitate. After stirring for 30 min, the solid was filtered and washed by water and dried to give compound B82 (140 mg, yield 100%).

A flask was charged with compound B82 (180 mg, 0.6 mmol), Lawesson's reagent (123 mg, 0.3 mmol) and anhydrous toluene (10 mL). The resulting mixture was heated to reflux and maintained at this temperature for 4 hrs. The reaction was monitored by LCMS. After completion of the reaction, the solvent was removed under reduced pressure, the crude product was purified by prep-TLC (EtOAc as eluent) to obtain compound B83 (70 mg, yield 47%).

A flask was charged with compound B83 (70 mg, 0.28 mmol), compound A76c (140 mg, 0.84 mmol) and EtOH (15 mL). The resulting mixture was heated to reflux and maintained at this temperature for 16 hrs. The reaction was monitored by LCMS. After completion of the reaction, the solvent was removed under reduced pressure, the crude product was purified by prep-TLC (PE:EA=3:1) to give compound B84 (80 mg, yield 90%).

A flask was charged with compound B84 (80 mg, 0.25 mmol) and POCl₃ (3 mL). The resulting mixture was heated to reflux and maintained at this temperature for 4 hrs. The reaction was monitored by LCMS. After completion of the reaction, most of POCl₃ was evaporated, the residue was diluted with ice-water, neutralized by saturated aq.NaHCO₃, extracted with EtOAc (30 mL×3), the organic layer was combined and washed with brine, dried over anhydrous sodium sulfate, the solvent was removed under reduced pressure to afford compound B85 (100 mg, crude yield 120.5%), The crude product was used directly in the next step.

The procedure for preparation of compound 1436 is similar to that of preparation of compound 1431. (48.3 mg, yield 18.6%). MS (ESI) m/z (M+H)⁺ 868.7.

### Example 15

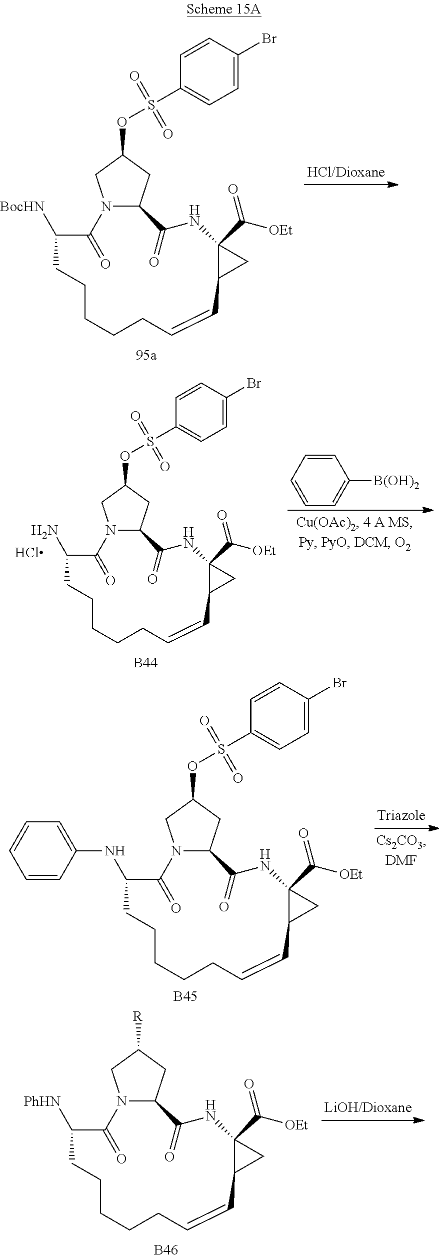

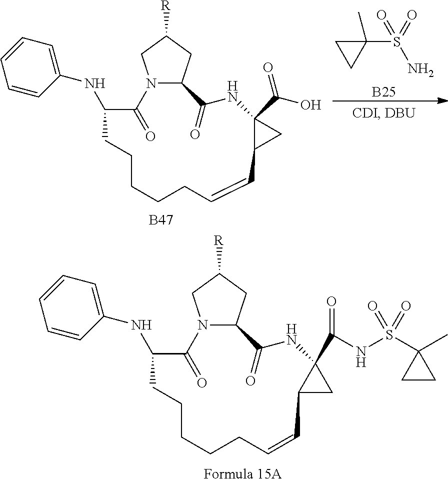

To a solution of compound 95a (430 mg, 0.60 mmol) in 12 mL of anhydrous DCM was added a solution of HCl/Dioxane (4N, 6 mL), the resulting solution was stirred at ambient temperature for 3 h. After the reaction complete, the solution was evaporated in vacuo to give the title compound B44 as a white solid. (392 mg, yield 100%) MS (EST) m/z (M+H)⁺ 611.9.

A mixture of compound B44 (391 mg, 0.6 mmol.), phenylboronic acid (221 mg, 1.8 mmol.), Cu(OAc)₂ (868 mg, 4.8 mmol.), pyridine (379 mg, 4.8 mmol.), pyridine N-Oxide (456 mg, 4.8 mmol.) and molecular sieves (4A) in dichloromethane (10 mL) was stirred for 12 h at room temperature under an atmosphere of oxygen. The reaction was monitored by LCMS. After completion of the reaction, the reaction mixture was diluted with ethyl acetate (30 mL) and filtered. The filtrate was washed with brine, dried over anhydrous sodium sulfate, concentrated in vacuo. The residue was purified with prep-TLC to give compound B45 (300 mg, yield 72%). MS (ESI) m/z (M+H)⁺ 689.1.

To a solution of macrocyclic precursor B45 and triazole in DMF was added cesium carbonate. The reaction mixture was stirred at 70° C. overnight. The reaction was extracted with EtOAc, washed with brine and concentrated to give a yellow residue. The compound B46 was isolated by prep-HPLC. The following compounds were prepared in this step.

TABLE 16
Compounds B46a-B46e.

| Compound | Structure | Yield |
|---|---|---|
| B46a | 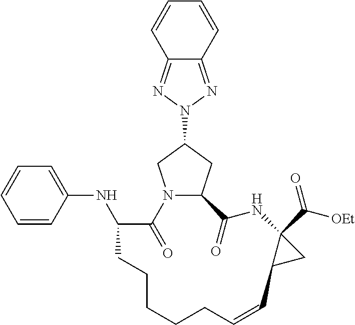 | 90 mg, yield 74%. MS (ESI) m/z (M + H)⁺ 571.1. |
| B46b | 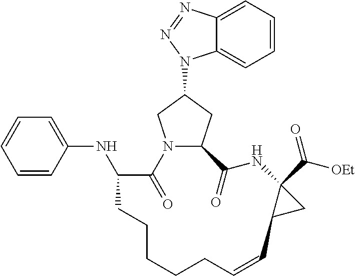 | 28 mg, yield 24%. MS (ESI) m/z (M + H)⁺ 571.1. |
| B46c | 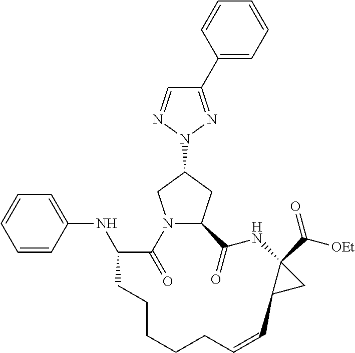 | 80 mg, yield 70%. MS (ESI) m/z (M + H)⁺ 597.1. |
| B46d | 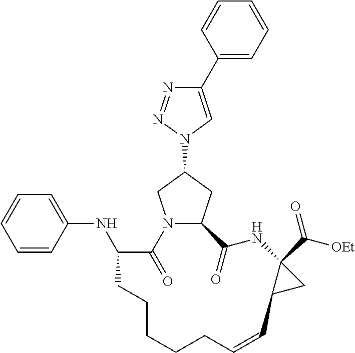 | 15 mg, yield 14%. MS (ESI) m/z (M + H)⁺ 597.1. |
| B46e | 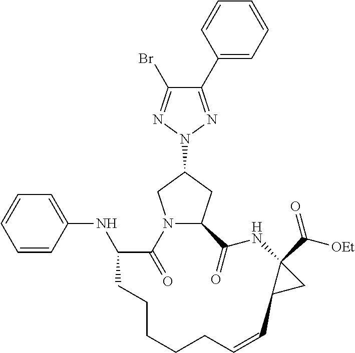 | 42 mg, yield 82%. MS (ESI) m/z (M + H)⁺ 676.1. |

LiOH (1N) was added to a solution of compound B46 in dioxane. The resulting solution was stirred at ambient temperature overnight. The reaction mixture was adjusted to pH=3 with citric acid, then extracted with EtOAc and washed with brine. The organic layer was dried in vacuo to give compound B47.

A solution of compound B47 (1 eq.) and CDI (3 eq.) in DCM was stirred at reflux for 4 hours under nitrogen atmosphere, then sulfonamide (3 eq.) and DBU (3 eq.) was added. The resulting mixture was stirred at reflux overnight. The reaction solution was extracted with EtOAc, washed with brine and concentrated in vacuo. The final compound of Formula 15A was purified by pre-HPLC. Compounds 1501-1505 were prepared in this step.

TABLE 17
Compounds 1501-1505.

| Compound | Structure | Yield |
|---|---|---|
| 1501 | 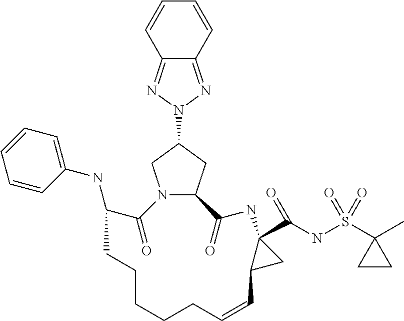 | 29.5 mg, yield 45%. MS (ESI) m/ z (M + H)⁺ 660.1. |
| 1502 | 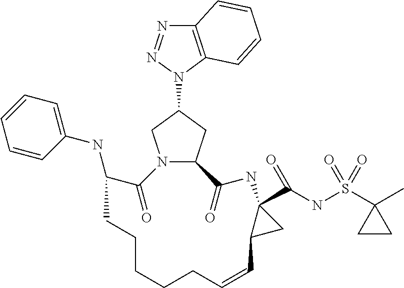 | 8.7 mg, yield 42%. MS (ESI) m/z (M + H)⁺ 660.1. |
| 1503 | 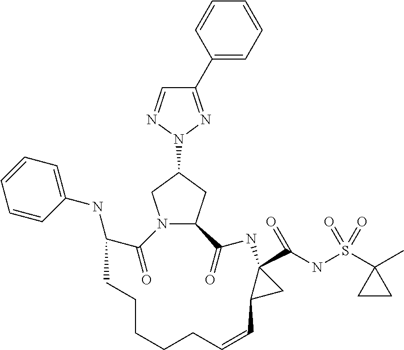 | 42 mg, yield 36%. MS (ESI) m/z (M + H)⁺ 686.3. |
| 1504 | 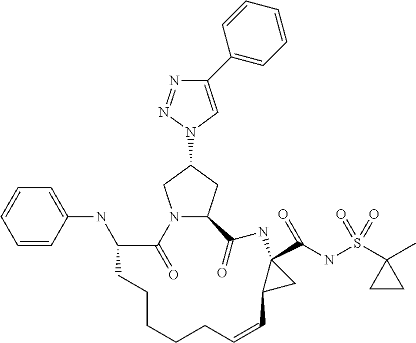 | 8.6 mg, yield 26%. MS (ESI) m/ z (M + H)⁺ 686.3. |
| 1505 | 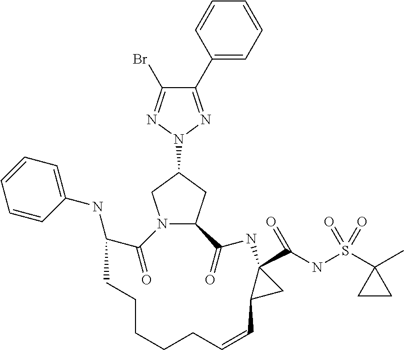 | 12 mg, yield 26%. MS (ESI) m/z (M + H)⁺ 764.2, 766.1. |

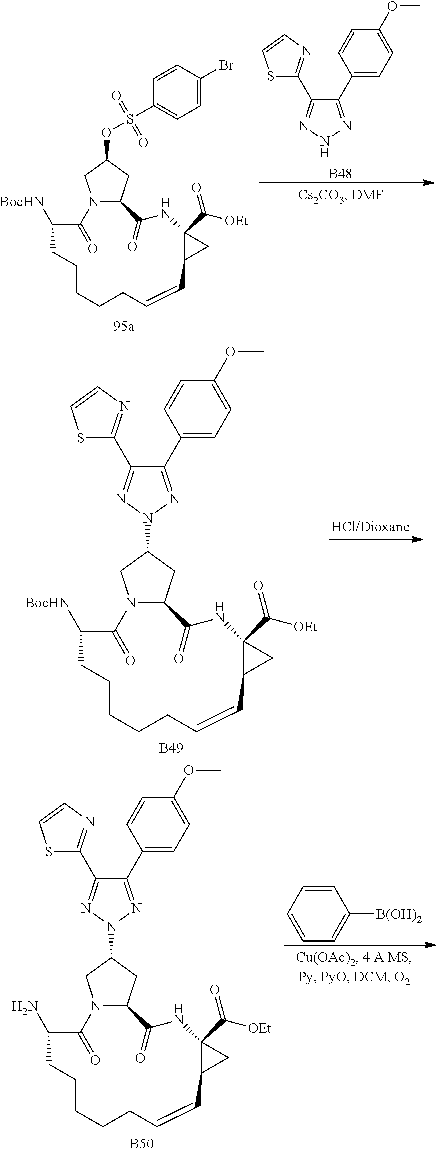

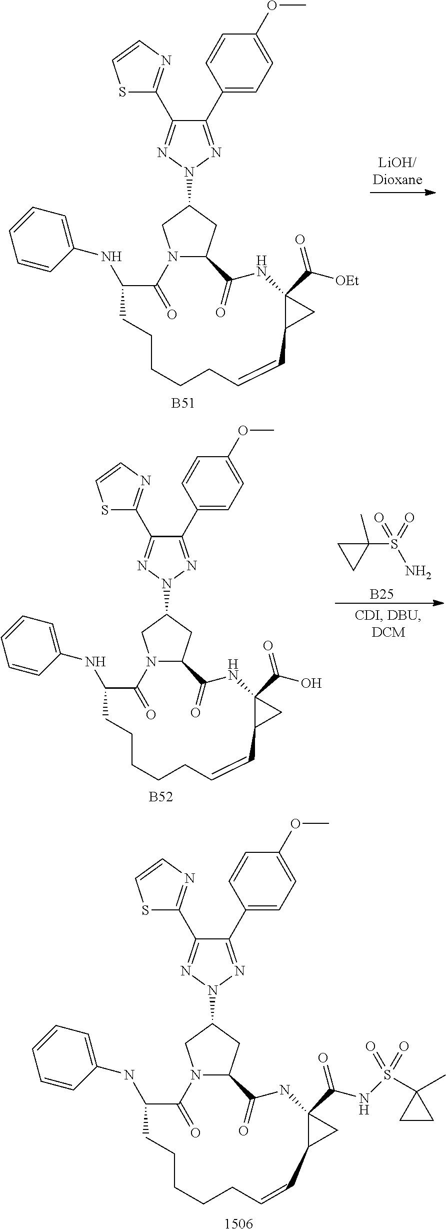

To a solution of macrocyclic precursor 95a (120 mg, 0.17 mmol.) and B48 (57 mg, 0.22 mmol.) in DMF was added cesium carbonate (218 mg, 0.67 mmol.). The reaction mixture was stirred at 70° C. overnight. The reaction was extracted with EtOAc, washed with brine and concentrated to give a yellow residue, which was purified by prep-HPLC to yield compound B49 (110 mg, yield 89%). MS (ESI) m/z (M+H)⁺ 734.2.

To a solution of compound B49 (110 mg, 0.15 mmol.) in 5 mL of anhydrous DCM was added a solution of HCl/Dioxane (4N, 5 mL), the resulting solution was stirred at ambient temperature for 3 h. After the reaction complete, the solution was concentrated by vacuo to give the title compound B50 as a light yellow solid (100 mg, yield 99%). MS (ESI) m/z (M+H)⁺ 634.2.

Compound B51 was prepared following the general procedure, and was obtained by prep-TLC as a light yellow solid (62 mg, yield 55%). MS (ESI) m/z (M+H)⁺710.3.

Compound B52 was prepared following the general procedure, and was freeze-dried as a light yellow solid (48 mg, yield 81%). MS (ESI) m/z (M+H)⁺ 682.2.

Compound 1506 was prepared following the general procedure. The final compound was purified by prep-HPLC. (30 mg, yield 53%). MS (ESI) m/z (M+H)⁺ 799.3. Compound 1506 can also be prepared using the same method for preparation of compound 1501.

### Example 16

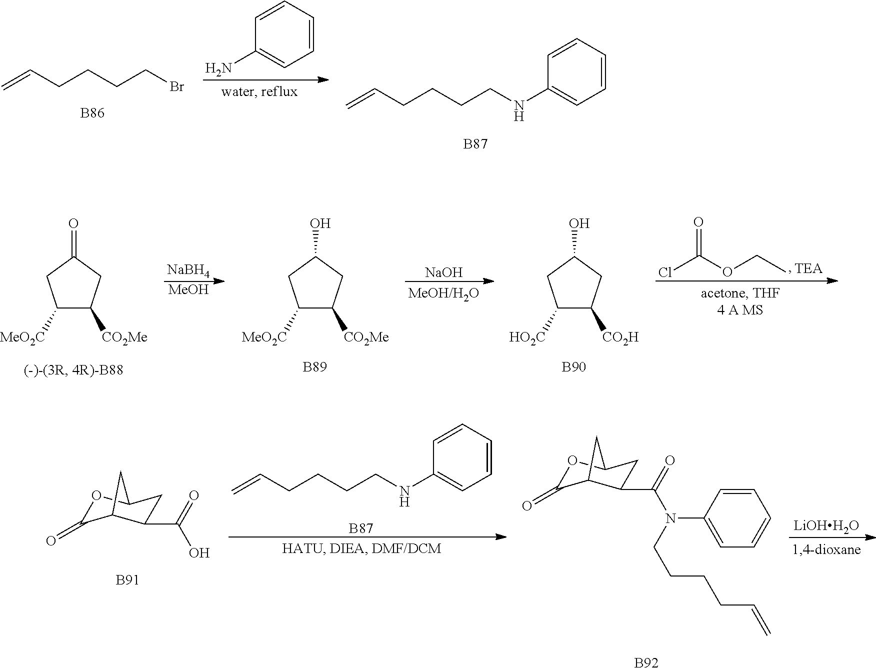

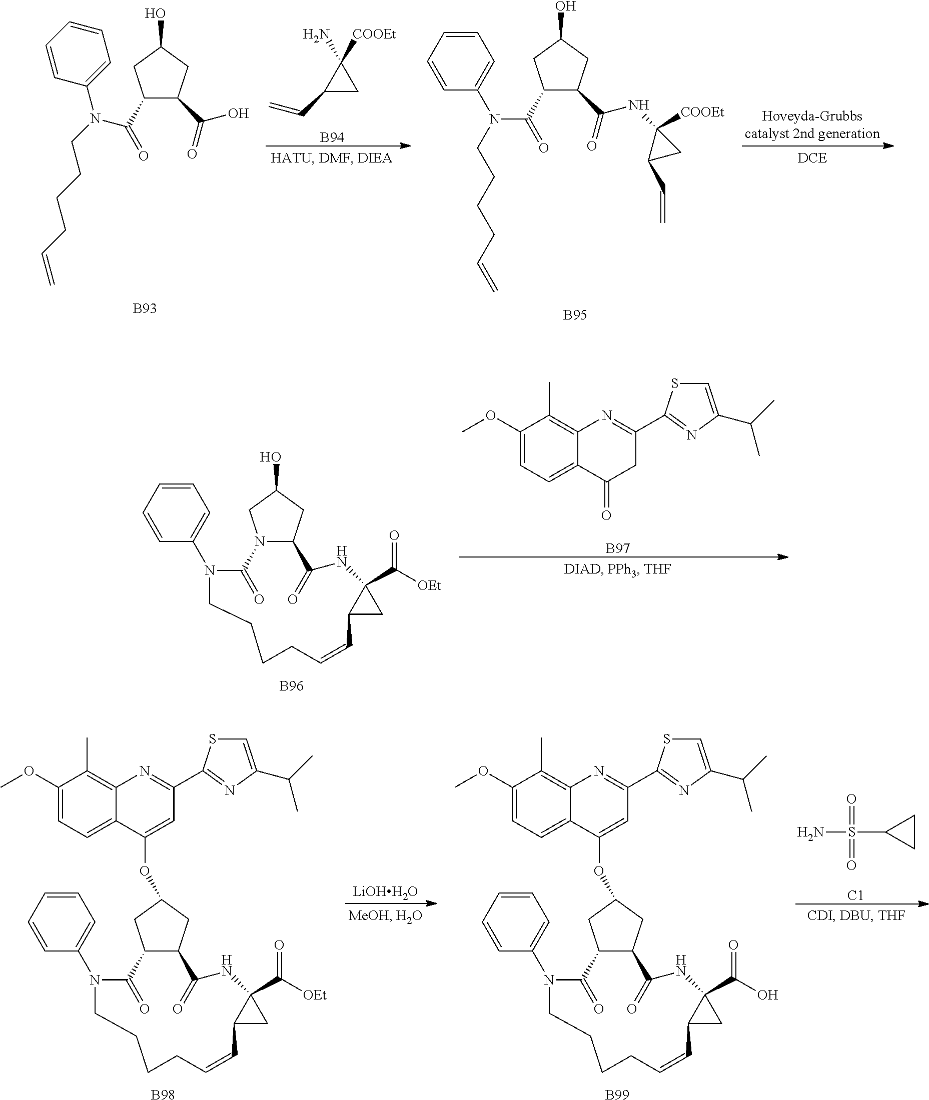

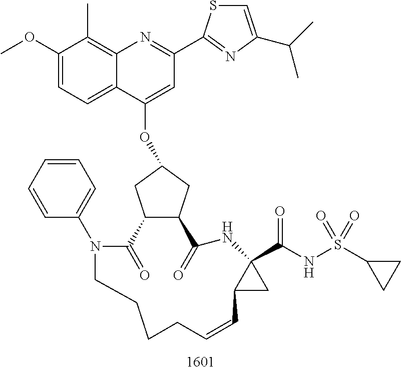

A mixture of aniline (10 g, 0.1 mol), 6-bromo-1-hexene B86 (8.7 g, 0.054 mol) and water (30 mL) was heated under reflux overnight. The mixture was cooled to room temperature, and basified with saturated aq. Na₂CO₃ to adjust pH to 10. The aqueous phase was extracted with CH₂Cl₂ (200 mL×3). The combined organic phase was dried over Na₂SO₄, concentrated in vacuo. The resulting residue was purified on silica gel column to provide compound B87 (3.3 g, yield 17.6%) as a light yellow.

Sodium borohydride (1.23 g, 3.25 mmol) was added to a stirred solution of compound B88 (5 g, 2.5 mmol) in methanol (100 mL) at 0° C. After 1 h, the reaction was quenched with 200 mL of brine, concentrated, and extracted with ethyl acetate, dried over Na₂SO₄, concentrated in vacuo to give compound B89 (4.1 g, yield 81%, [c]ₚ of −64) as yellow oil.

Aqueous sodium hydroxide (1 M, 80 mL) was added to a stirred solution of compound B89 (4 g, 19.8 mmol) in methanol (80 mL) at room temperature. After stirring for 4 hrs, the reaction mixture was neutralized with aq. HCl (3 M), evaporated and co-evaporated with toluene several times. The resulting residue was washed with anhydrous THF (200 mL×3), and filtered. The filtrate was concentrated to give crude compound B90 as a white solid (3.4 g, crude yield 100%)

To a suspension of compound B90 (0.3 g, 1.72 mmol) in THF (6 mL) and acetone (6 mL), was added triethylamine (0.17 g, 1.72 mmol). The mixture was cooled to 0° C., and ethyl chloroformate (0.472 g, 4.35 mmol) was added dropwise. The mixture stirred for further 2 hours at 0° C. to 5° C. The reaction was warmed to r.t. and stirred overnight. The mixture was filtered and the solids washed with THF (25 mL). The filtrate was concentrated to give crude compound B91 (0.18 g, yield 67%) as a light yellow viscous liquid.

A mixture of compound B91 (0.18 g, 1.15 mmol), compound B87 (0.244 g, 1.38 mmol), HATU (0.88 g, 2.3 mmol), DMF (2.5 mL), CH₂Cl₂ (6 mL) and DIEA (0.29 g, 2.3 mmol) was stirred at r.t. overnight. The mixture was diluted with CH₂Cl₂ (20 mL), washed successively with 0.1 M aq. HCl (10 mL×3), saturated aq. NaHCO₃ (10 mL) and water (10 mL). The organic phase was dried over Na₂SO₄, and concentrated. The residue was purified by column chromatography to give compound B92 (0.1 g, yield 29%) as a light yellow oil.

To a solution of LiOH.H₂O (52 mg, 1.24 mmol) in H₂O (5 mL) was added a solution of compound B92 (0.26 g, 0.83 mmol) in 1,4-dioxane (8 mL) dropwise at r.t. The mixture was stirred at r.t for 5 hrs, then acidified by aq. HCl (1 M) to pH=2˜3. The aqueous layer was extracted with EtOAc (10 mL×3). The combined organic phase was dried over Na₂SO₄, concentrated to give compound B93 (0.27 g, yield 98%) as a viscous colorless oil.

To a solution of compound B93 (0.11 g, 0.332 mmol) in CH₂Cl₂ (5 mL) and DMF (1.5 mL) was added DIEA (0.176 mL, 0.996 mmol) and compound B94 (0.127 g, 0.664 mmol). The mixture was stirred at 0° C. for 10 min, followed by addition of HATU (0.25 g, 0.664 mmol) at 0° C. The mixture was stirred at r.t. overnight. After the completion of reaction, the solvent was evaporated. The residue was purified by prep-TLC to give compound B95 (0.1 g, yield 70%) as a light yellow oil.

To the solution of compound B95 (0.18 g, 0.384 mmol) in DCE (2300 ml) was added Hoveyda-Grubbs catalyst 2^{nd} generation (20 mg, 0.0319 mmol). The mixture was degassed three times, then heated under reflux at a nitrogen atmosphere overnight. After the completion of reaction, the solvent was evaporated. The residue was purified by column chromatography to afford compound B96 (0.16 g, yield 94.6%) as a light yellow oil.

To a mixture of compound B96 (50 mg, 0.114 mmol), PPh₃ (77.4 mg, 0.295 mmol), compound B97 (61 mg, 0.194 mmol) in anhydrous THF (4 mL) was added DIAD (64.7 mg, 0.331 mmol) dropwise at r.t. The mixture was stirred overnight. After the completion of reaction, the reaction was quenched with 10 mL of water. The aqueous phase was extracted with EtOAc (10 mL×3). The combined organic phase was dried over Na₂SO₄, concentrated in vacuo. The residue was purified by column chromatography to provide compound B98 (20 mg, yield 24%) as a white solid.

A mixture of compound B98 (40 mg, 0.0543 mmol), LiOH.H₂O (46 mg, 1.086 mmol), THF (1 mL), MeOH (1 mL) and H₂O (1 mL) was stirred at 35˜45° C. for 24 hrs. After the completion of reaction, the mixture was neutralized with aq. HCl (1 M) to pH=5. The aqueous layer was extracted with EtOAc (20 mL×3). The combined organic phase was dried over Na₂SO₄, concentrated in vacuo to give compound B99 (39 mg, yield 100%) as a light yellow solid.

A mixture of compound B99 (39 mg, 0.056 mmol) and CDI (55 mg, 0.34 mmol) in anhydrous THF (5 mL) was heated under reflux for 4 hours, then cooled to r.t., sulfonamide C1 (20 mg, 0.169 mmol) and DBU (69 mg, 0.453 mmol) was added. The mixture was stirred at 50° C. overnight. The reaction was monitored by LCMS. After the completion of reaction, the mixture was quenched with 10 mL of saturated aq. NaCl. The aqueous layer was extracted with EtOAc (20 mL×3). The combined organic phase was dried over Na₂SO₄, concentrated in vacuo. The residue was purified by prep-HPLC to afford compound 1601 (20 mg, yield 43.5%) as a light yellow solid. ¹H NMR (CDCl₃, 300 MHz) δ: 11.0 (s, 1H), 7.92 (d, J=9Hz, 1H), 7.48-7.46 (m, 3H), 7.24 (d, J=2.7 Hz, 1H), 7.15 (d, J=9 Hz, 1H), 6.96 (s, 1H), 6.38 (s, 1H), 5.66-5.64 (m, 1H), 5.06-4.86 (m, 3H), 4.07 (s, 3H), 3.43-3.12 (m, 3H), 2.90-2.86 (m, 2H), 2.64 (s, 4H), 2.42 (s, 1H), 2.30-2.22 (m, 1H), 2.18-2.02 (m, 2H), 1.95-1.65 (m, 3H), 1.61-1.40 (m, 4H), 1.25 (d, 6H), 1.22-1.15 (m, 3H), 1.11-0.90 (m, 2H). MS (ESI) m/z (M+H)⁺: 812.1.

### Example 17

### 17.1 Synthesis of Compound 1701-1707

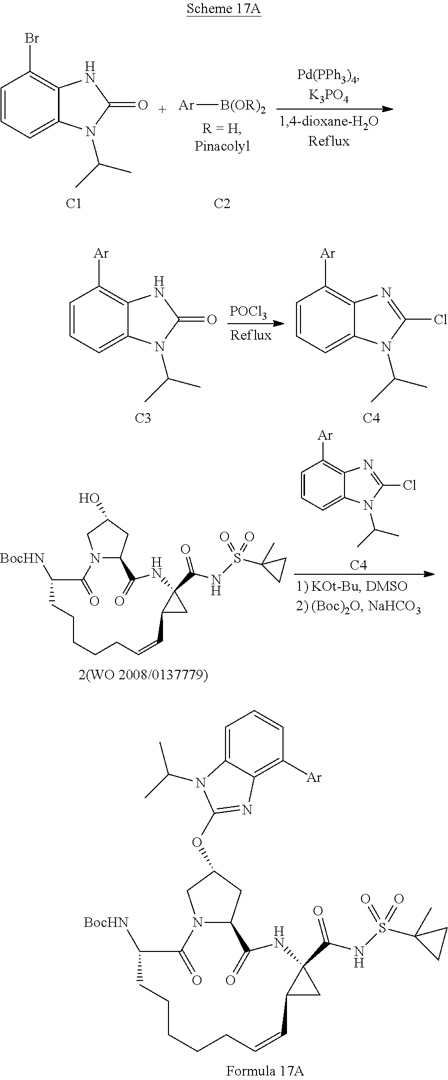

A flask charged with compound C1 (1 eq.), compound C2 (1 eq.), K₃PO₄ (55 mg, 0.26 mmol, 1.5 eq.) and 2 mL of 1,4-dioxane and 100 μL of water. The flask was purged with nitrogen and then Pd(PPh₃)₄ (0.01 eq.) was added. The mixture was heated to reflux, and stirred for 18 hrs. LCMS showed the reaction completed. The mixture was cooled to r.t and water (5 mL) was added thereto, followed by extraction with ethyl acetate (50 mL×3), the organic layers were combined, washed with brine, dried over anhydrous sodium sulfate, concentrated under reduced pressure, the residue was purified by prep-TLC to give compound C3.

The following borates were coupled with compound C1 using the procedure described, but it is not limited to those borates:

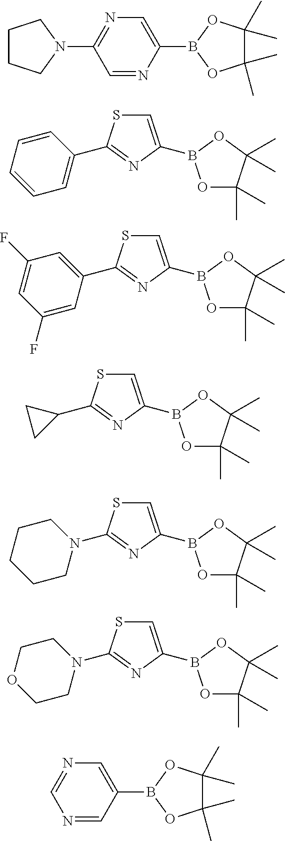

Compound C3 was dissolved in POCl₃ and then the mixture was refluxed under nitrogen. After the completion and cooling to r.t., the reaction mixture was taken up with ice-water, neutralized with ammonia under cooling, extracted with EtOAc (30 mL×3). The combined organic layers were washed with brine, dried over anhydrous Na₂SO₄ and concentrated to give a crude compound C4, which was used directly in next step.

To a solution of compound 2 (1 eq.) in 2 mL of DMSO was added KOt-Bu (5 eq.) portionwise at ambient temperature, the mixture was then stirred for 2 hrs at ambient temperature. Subsequently, general compound C4 (1.1 eq.) was added, the resulting mixture was stirred at r.t. for 20 hrs. The reaction was monitored by LCMS indicating the coupling product with loss of the Boc group. The stirring mixture was cooled in an ice-water bath and acidified by addition of aq. HCl (2 M) to pH=7-8. Subsequently, Boc₂O (1.5 eq.) and NaHCO₃ (1.5 eq.) were added. The mixture was stirred for another 2 hrs and then was acidified to pH=5˜6 with aq. HCl (0.1 M) and extracted with ethyl acetate. The organic layers were combined, washed by brine, dried over anhydrous sodium sulfate, concentrated under reduced pressure, and the resulting residue was purified by prep-TLC or prep-HPLC to provide a compound of general formula 17A. Compounds 1701-1707 were prepared using Scheme 17A.

TABLE 18
Compounds 1701-1707.

| Compound | Structure | Yield |
|---|---|---|
| 1701 | 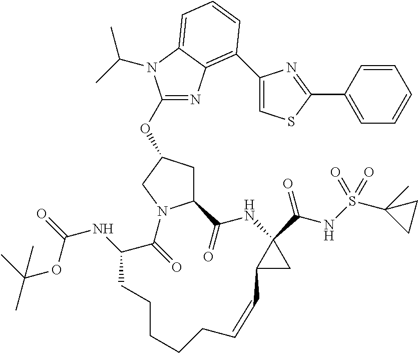 | 13.6 mg, yield 30%. MS (ESI) m/z (M + H)⁺ 900.4 |
| 1702 | 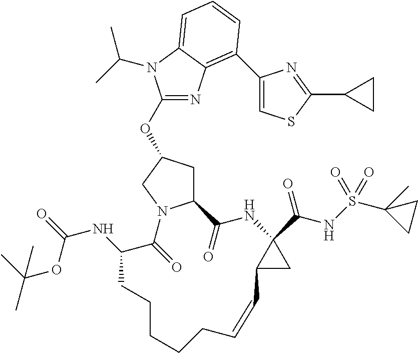 | 15.4 mg, yield 35%. MS (ESI) m/z (M + H)⁺ 864.3. |
| 1703 | 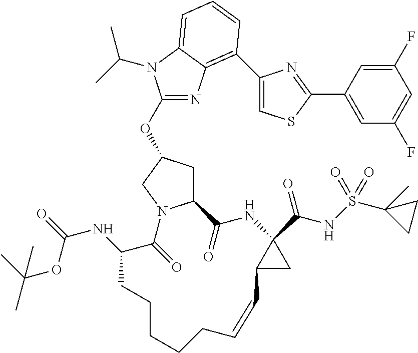 | 12 mg, yield 25%. MS (ESI) m/z (M + H)⁺ 936.3. |
| 1704 | 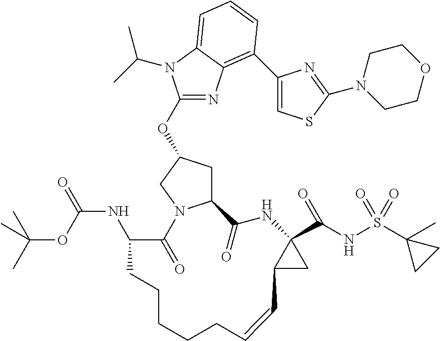 | 6.5 mg, yield 15%. MS (ESI) m/z (M + H)⁺ 909.4 |
| 1705 | 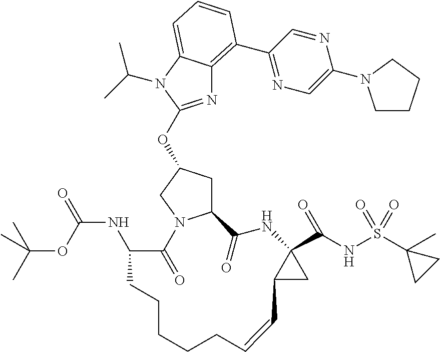 | 12 mg, yield 27%. MS (ESI) m/z (M + H)⁺ 888.4 |
| 1706 | 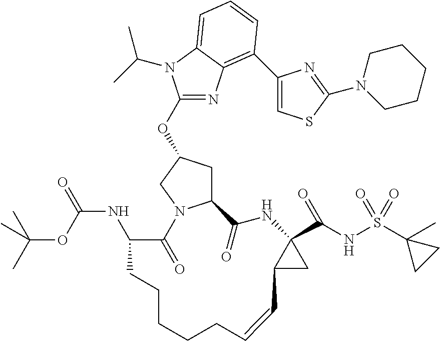 | 7 mg, yield 13%. MS (ESI) m/z (M + H)⁺ 907.5 |
| 1707 | 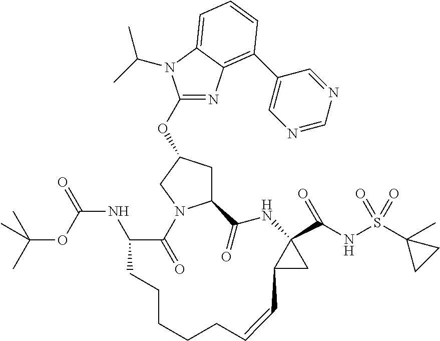 | 8.4 mg, yield 15%. MS (ESI) m/z (M + H)⁺ 819.3. |

### 17.2 Synthesis of Compound 1708

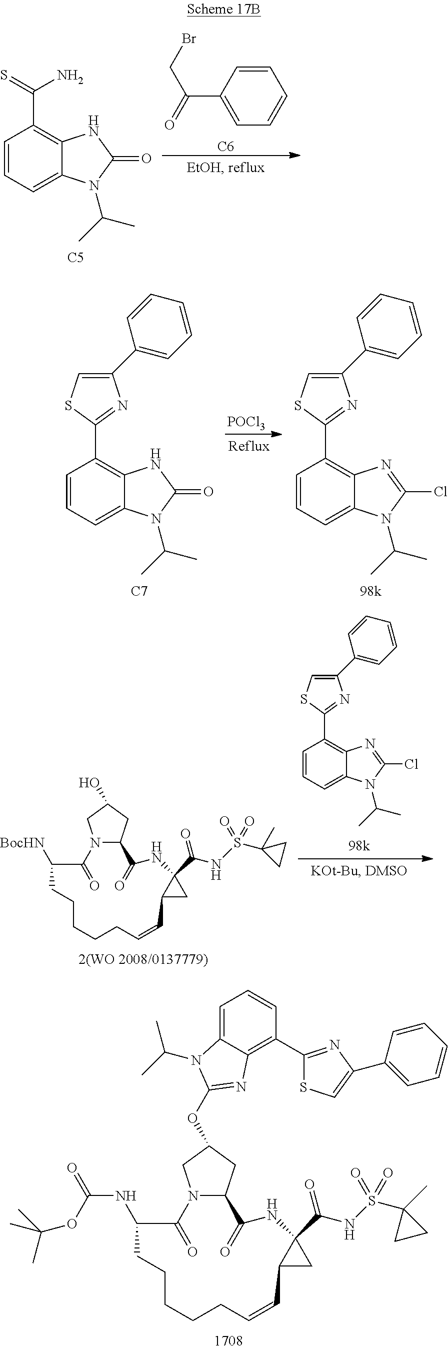

To a solution of compound C5 (400 mg, 1.7 mmol) in EtOH (30 mL) was added compound C6 (677 mg, 3.4 mmol). The mixture was refluxed under nitrogen. After completion of the reaction the solvent was evaporated under reduced pressure to afford a crude product. The crude product was purified by column chromatography on silica gel (PE/EA=3:1) to afford compound C7 (470 mg, yield 83%). MS (ESI) m/z (M+H)⁺ 336.

Compound C7 (400 mg, 1.19 mmol) was dissolved in POCl₃ (7 mL) and the resulting mixture was refluxed under nitrogen. After completion of reaction, excess of POCl₃ was removed, and then the mixture was taken up with ice-water, neutralized with ammonia under cooling, and then extracted with EtOAc (20 mL×3). The combined organic layers were washed with brine, dried over anhydrous Na₂SO₄ and concentrated. The resulting crude product 98k was used directly in next step (409 mg, yield 97%).

To a solution of compound 2 (250 mg, 0.43 mmol) in DMSO (20 mL) was added KOt-Bu (202 mg, 1.81 mmol), the resulting mixture was stirred at 0° C. for 1 h under nitrogen. Subsequently, compound 98k (151 mg, 0.43 mmol) was added and the reaction mixture was stirred at r.t. for 1.5 hrs. After completion of the reaction, the reaction was quenched by ice water. The pH was adjusted to pH=4-5 by addition of aq. HCl (1 M) and the resulting mixture was extracted with EtOAc (20 mL×3). The combined organic layers were washed with brine, dried over anhydrous Na₂SO₄ and concentrated in vacuo. The residue was purified by prep-HPLC to afford Compound 1708 as white solid (140 mg, yield 37%). MS (ESI) m/z [M]⁺ 900.3.

### 17.3 Synthesis of Compounds 1709-1717

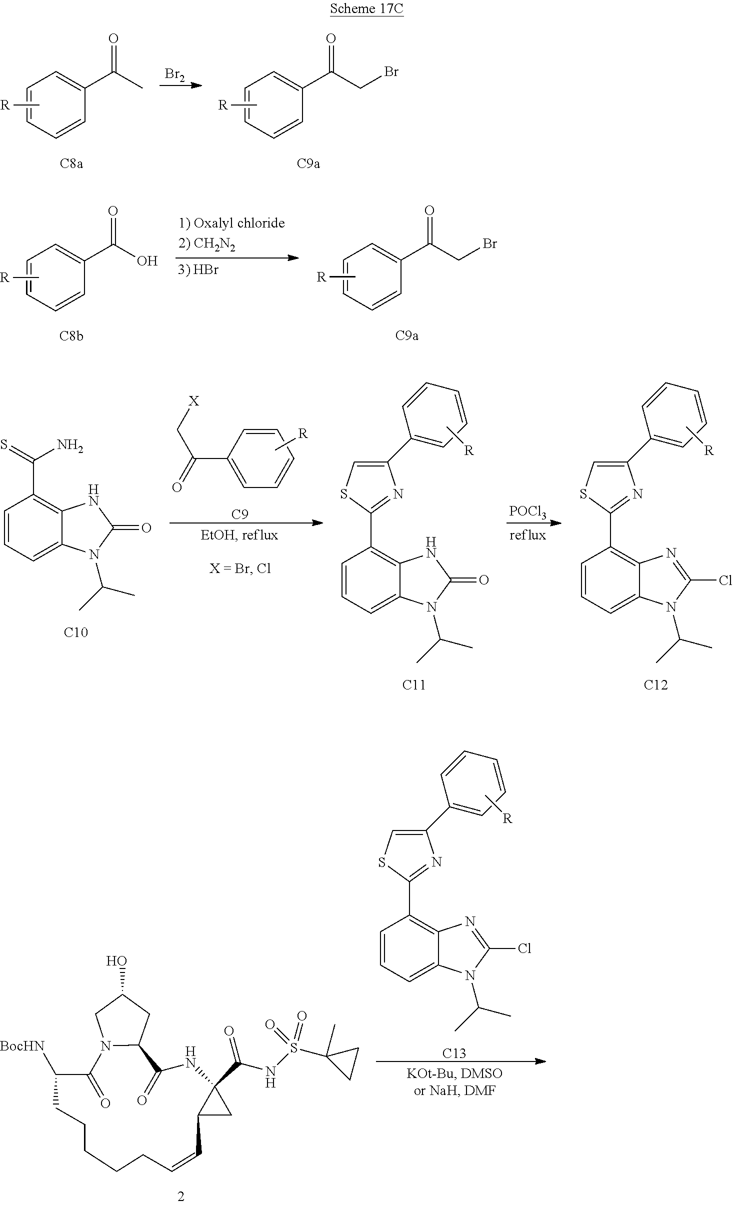

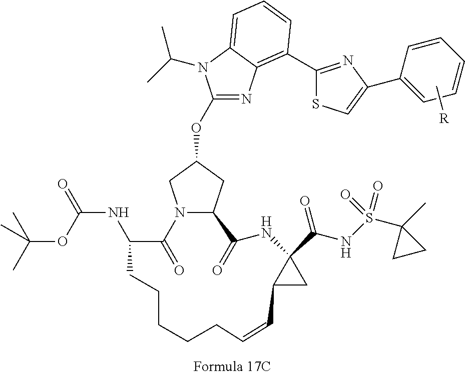

To a solution of general compound C8a (1 eq.) in glacial acetic acid (or chloroform) was added bromine (1 eq.) dropwise and the mixture was stirred at room temperature for 2 hrs. After the reaction was completed, the solvent was evaporated under reduced pressure. The crude product general compound C9a was used for the next step without further purification. The following compounds were prepared:

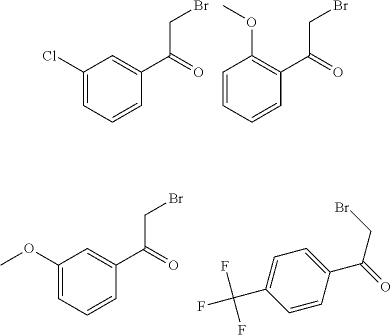

To a solution of general compound C8b (1 eq.) in anhydrous CH₂Cl₂ was added oxalyl chloride (1.1 eq.) and one drop of DMF at 0° C. The resulting mixture was stirred for 2 hrs at room temperature and then the mixture was then concentrated in vacuo. The resulting residue was dissolved in anhydrous CH₂Cl₂ and to this solution was added freshly prepared diazomethane (diethyl ether solution, 2.5 eq.) dropwise at 0° C. After the addition of diazomethane completed, the mixture was stirred at 0° C. for 30 min. TLC indicated disappearance of the starting material. To this solution, an aqueous solution of hydrobromic acid (48%, 4 eq.) was added. The mixture was maintained at 0° C. during the addition of hydrobromic acid. The mixture was allowed to warm to r.t. and stirred overnight. Subsequently, the mixture was treated with saturated aqueous NaHCO₃ to adjust the pH to 7. The layers were separated and the aqueous layer was extracted with EtOAc (30 mL×2). The combined organic phase was washed with brine (30 mL), dried over sodium sulfate, and concentrated in vacuo to provide general compound C9a, which was used in the next step without further purification. The following compounds were prepared:

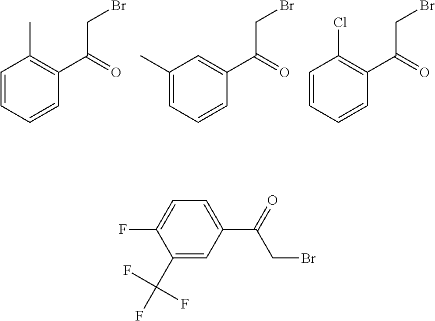

The following compounds are commercial available.

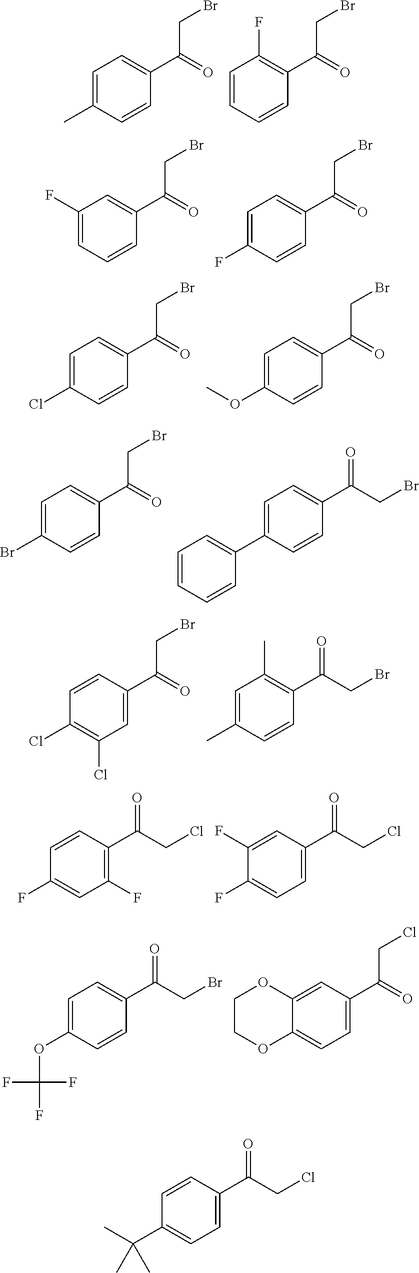

To a solution of compound C10 (1 eq.) in EtOH (2 mL) was added general compound C9 (1.2 eq.). The mixture was refluxed under nitrogen and after completion of the reaction the solvent was removed. The resulting mixture was diluted with EtOAc (60 mL) and then washed with water (20 mL), brine (10 mL×2), dried over anhydrous Na₂SO₄, and then concentrated in vacuo. The residue was purified by prep-TLC to afford compound C11.

The preparation of compound C12 followed the procedure for preparing compound 98k as described above under section 17.2.

The preparation of compound of formula 17C followed the same procedure for preparing compound 1708. The t-BuOK in DMSO was used with this procedure for preparation of following compounds:

TABLE 19
Compounds 1709-1711.

| Compound | Structure | Yield |
|---|---|---|
| 1709 | 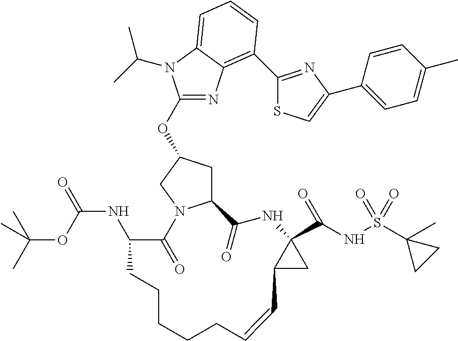 | 13.1 mg, yield 24%. MS (ESI) m/z (M + H)⁺ 914.5 |
| 1710 | 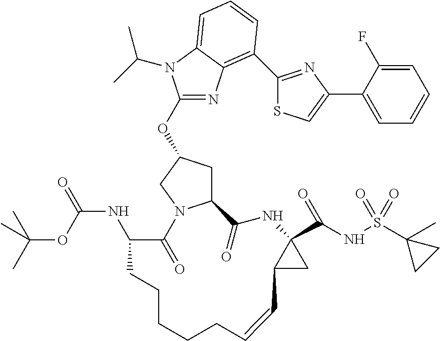 | 11.8 mg, yield 20%. MS (ESI) m/z (M + H)⁺ 918.5 |
| 1711 | 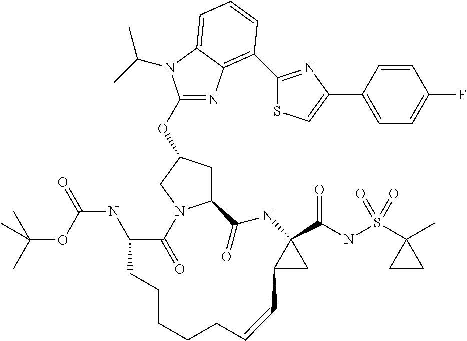 | 6.5 mg, yield 14%. MS (ESI) m/z (M + H)⁺ 918.2 |

General Procedure for preparing compound 1708 was adopted for preparation of compounds 1712-1717 using NaH in DMF.

TABLE 20
Compounds 1712-1717.

| | | |
|---|---|---|
| 1712 | 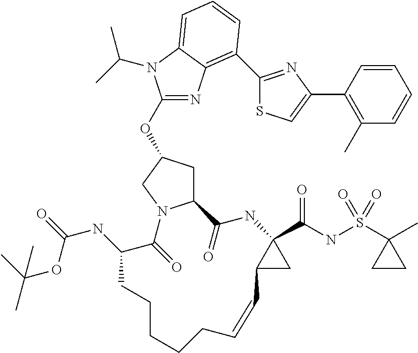 | 12 mg, yield 25%. MS (ESI) m/z (M + H)⁺ 914.4 |
| 1713 | 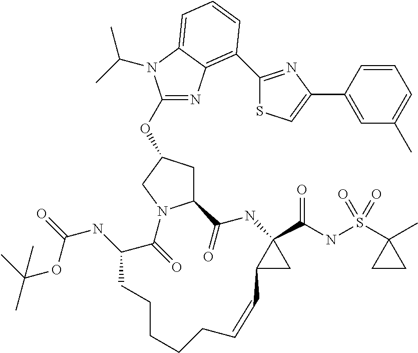 | 15 mg, yield 30%. MS (ESI) m/z (M + H)⁺ 914.4 |
| 1714 | 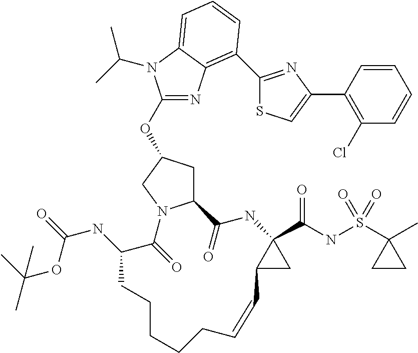 | 15.8 mg, yield 20%. MS (ESI) m/z (M + H)⁺ 934.2 |
| 1715 | 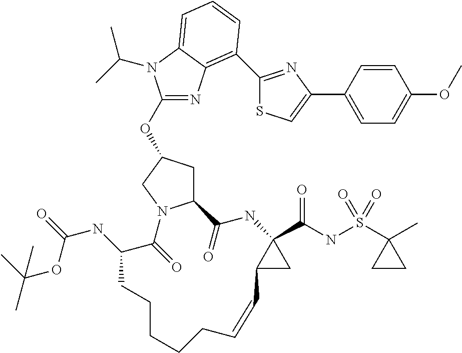 | 13.6 mg, yield 17%. MS (ESI) m/z (M + H)⁺ 930.2 |
| 1716 | 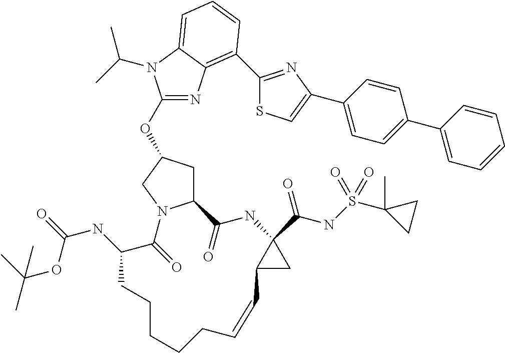 | 15.1 mg, yield 18%. MS (ESI) m/z (M + H)⁺ 976.2 |
| 1717 | 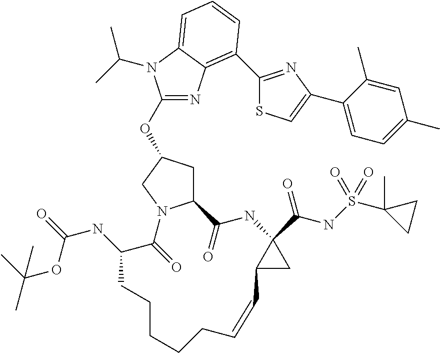 | 16 mg, yield 20%. MS (ESI) m/z (M + H)⁺ 928.2 |

### 17.4 Synthesis of Compounds 1718-1727

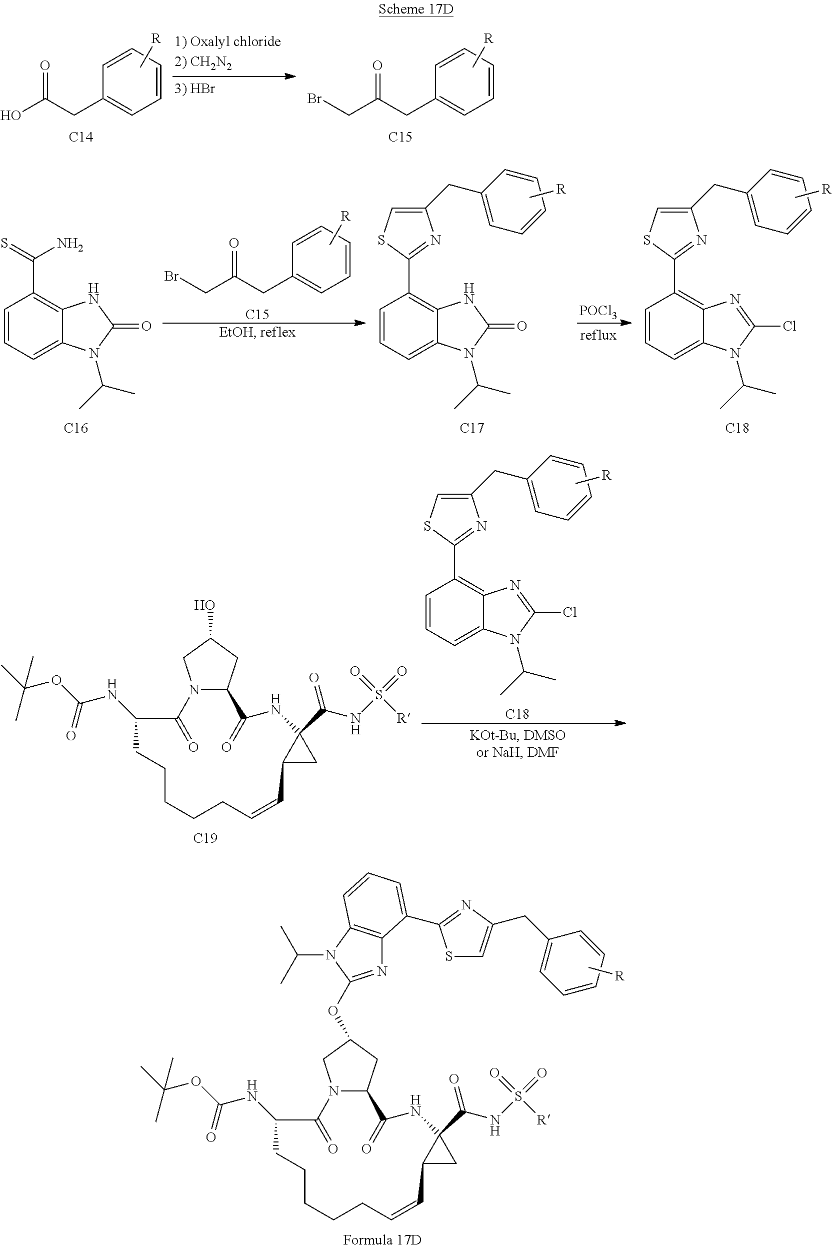

To a solution of general compound C14 (1 eq.) in anhydrous CH₂Cl₂ was added oxalyl chloride (1.1 eq.) and one drop of DMF at 0° C. The resulting mixture was stirred for 2 hrs at room temperature. Then the mixture was concentrated in vacuo and the residue was dissolved in anhydrous CH₂Cl₂. To the solution was added freshly prepared diazomethane (diethyl ether solution, 2.5 eq.) dropwise at 0° C. TLC indicated disappearance of the starting material. To this solution, an aqueous solution of hydrobromic acid (48%, 4 eq.) was added, the mixture was maintained at 0° C. during the addition of hydrobromic acid. The reaction was allowed to warm to r.t. and stirred overnight. The reaction was treated with saturated aqueous NaHCO₃ to adjust pH to 7. The layers were separated. The aqueous layer was extracted with EtOAc (30 mL×2). The combined organic phase was washed with brine (30 mL), dried over sodium sulfate, and concentrated in vacuo to provide general compound C15, which was used in the next step without further purification. The following compounds were prepared:

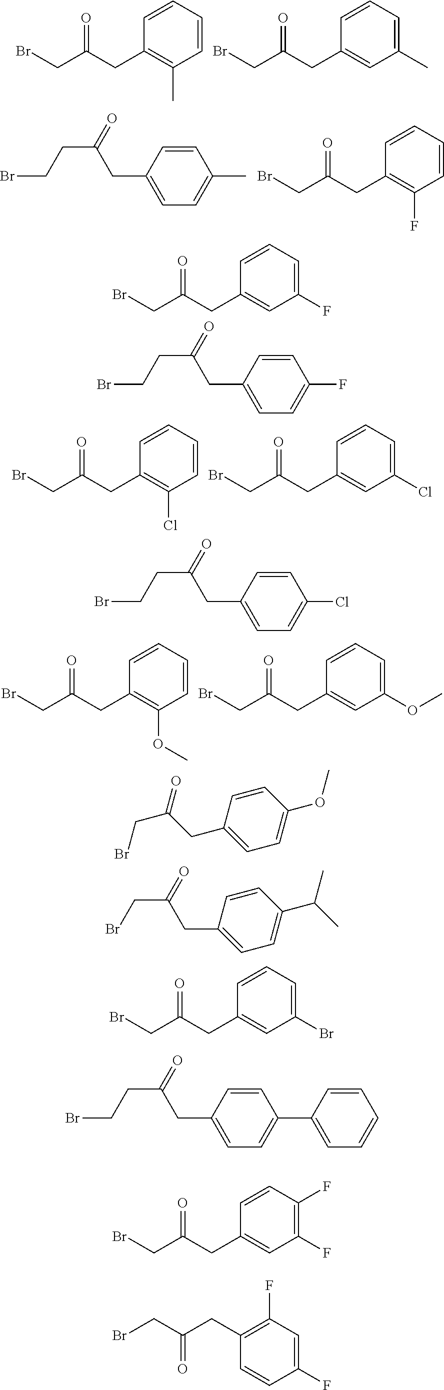

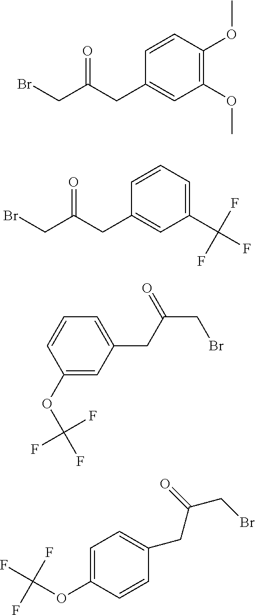

To a solution of compound C16 (1 eq.) in EtOH (2 mL) was added general compound C15 (1.2 eq.). The mixture was refluxed under nitrogen and after completion of the reaction the solvent was removed. The resulting mixture was diluted with EtOAc (60 mL) and then washed with water (20 mL), brine (10 mL×2), dried over anhydrous Na₂SO₄, and then concentrated in vacuo. The residue was purified by prep-TLC to give compound C17.

The preparation of general compound C18 followed the procedure for preparing compound 98k as described above under section 17.2.

The preparation of the compound of formula 17D followed the same procedure for preparing compound 1708. The t-BuOK in DMSO was used with this procedure for preparation of compounds 1718-1723.

TABLE 21
Compounds 1718-1723.

| Compound | Structure | Yield |
|---|---|---|
| 1718 | 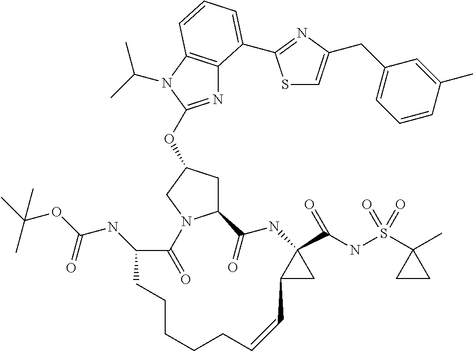 | 31.2 mg, yield 40%. MS (ESI) m/z (M + H)⁺ 928.4 |
| 1719 | 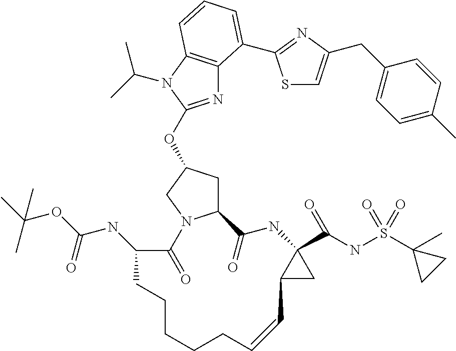 | 34.7 mg, yield 44%. MS (ESI) m/z (M + H)⁺ 928.3 |
| 1720 | 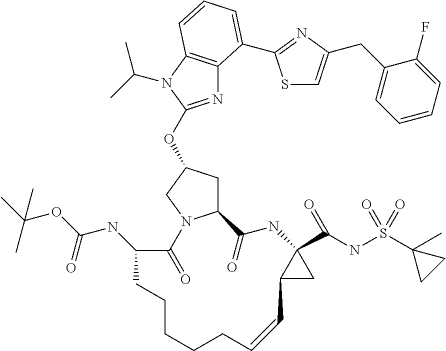 | 32.3 mg, yield 40%. MS (ESI) m/z (M + H)⁺ 932.3 |
| 1721 | 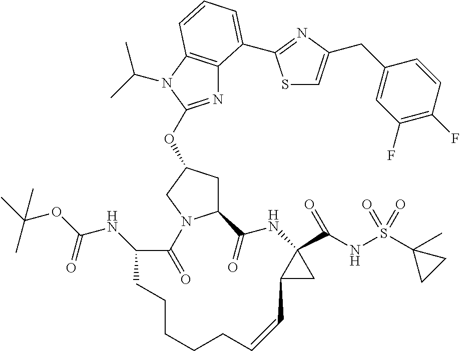 | 13 mg, yield 20%. MS (ESI) m/z (M + H)⁺ 950.3 |
| 1722 | 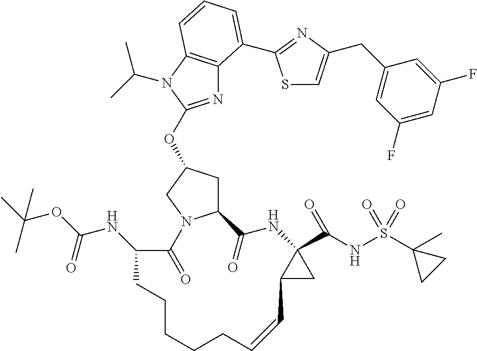 | 12 mg, yield 19%. MS (ESI) m/z (M + H)⁺ 950.3 |
| 1723 | 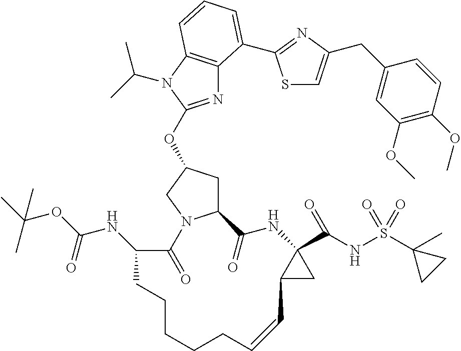 | 10 mg, yield 15%. MS (ESI) m/z (M + H)⁺ 974.4 |

The preparation of the compound of formula 17D followed the same procedure for preparing compound 1708. This method using NaH and DMF was adopted for preparation of compounds 1724-1727.

TABLE 22
Compounds 1724-1727.

| Compound | Structure | Yield |
|---|---|---|
| 1724 | 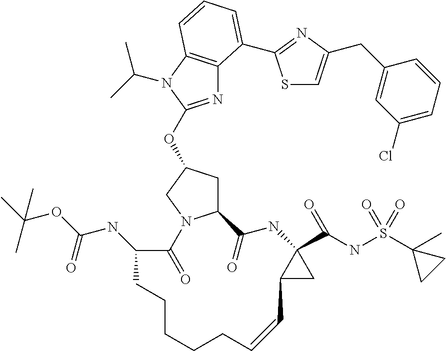 | 18.2 mg, yield 28%. MS (ESI) m/z (M + H)⁺ 948.3 |
| 1725 | 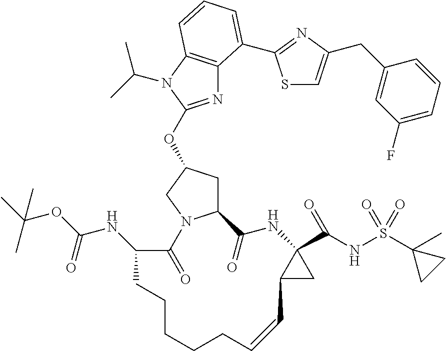 | 11.2 mg, yield 19%. MS (ESI) m/z (M + H)⁺ 932.2. |
| 1726 | 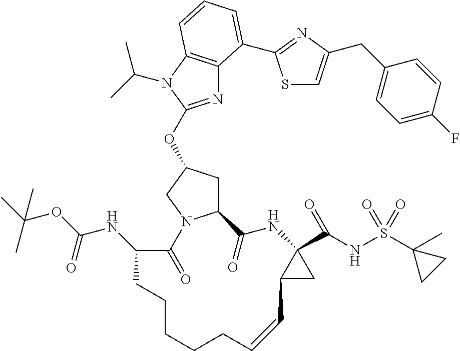 | 18.5 mg, yield 31%. MS (ESI) m/z (M + H)⁺ 932.2. |
| 1727 | 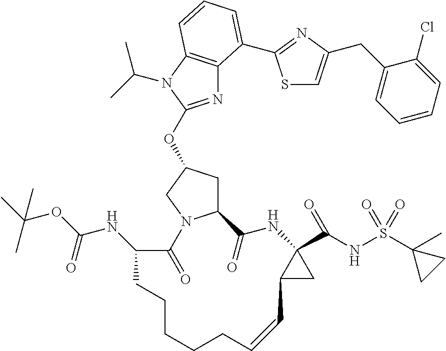 | 12.3 mg, yield 19%. MS (ESI) m/z (M + H)⁺ 948.3 |

### 17.5 Synthesis of Compounds 1728-1729

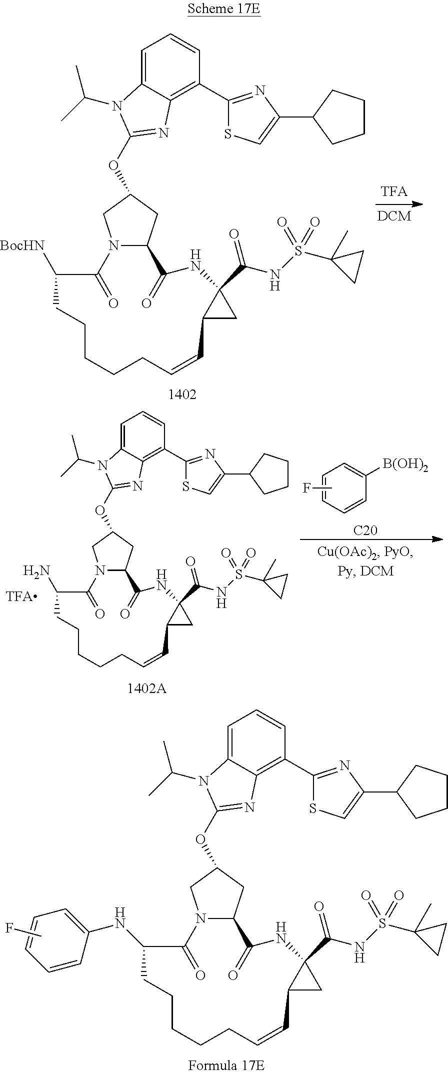

To a solution of compound 1402 (300 mg, 0.3 mmol) in 8 mL of dichloromethane was added 2 mL of trifluoroacetic acid, the resulting solution was stirred at room temperature for 2 hours. After the reaction completion, the solution was evaporated in vacuo to give the title compound 1402A as a colorless solid which was used directly in the next step.

A mixture of compound 1402A (250 mg, 0.3 mmol.), compound C20 (125 mg, 0.9 mmol.), Cu(OAc)₂ (162 mg, 0.9 mmol.), pyridine (240 mg, 3 mmol.), pyridine N-oxide (300 mg, 3 mmol.) and molecular sieves (4A, 1 g) in dichloromethane (15 mL) was stirred for 2 days at room temperature under an atmosphere of oxygen. The reaction was monitored by LCMS. After completion of the reaction, the mixture was diluted with ethyl acetate (50 mL) and filtered. The filtrate was washed with brine, dried over anhydrous sodium sulfate, and then concentrated in vacuo to provide a residue for further purification. The residue was purified by prep-HPLC to afford compound 1728 (39.2 mg, yield 14%). MS (ESI) m/z (M+H)⁺ 886.4. Compound 1729 was also prepared using the same method.

TABLE 23
Compounds 1728-1729.

| Compound | Structure | Yield |
|---|---|---|
| 1728 | 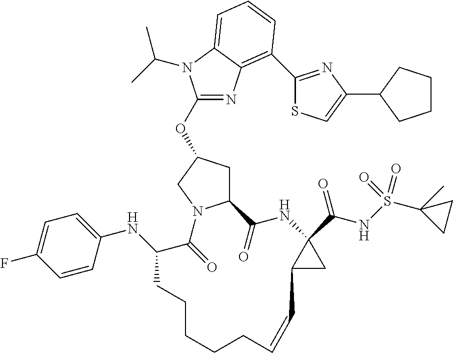 | MS (ESI) m/z (M + H)+ 886.4. |
| 1729 | 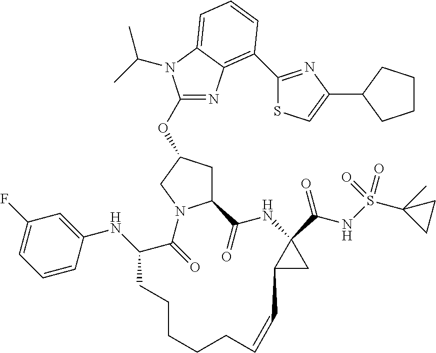 | 25 mg, yield 23%. MS (ESI) m/z (M + H)⁺ 886.2. |

### 17.6 Synthesis of Compounds 1730-1732

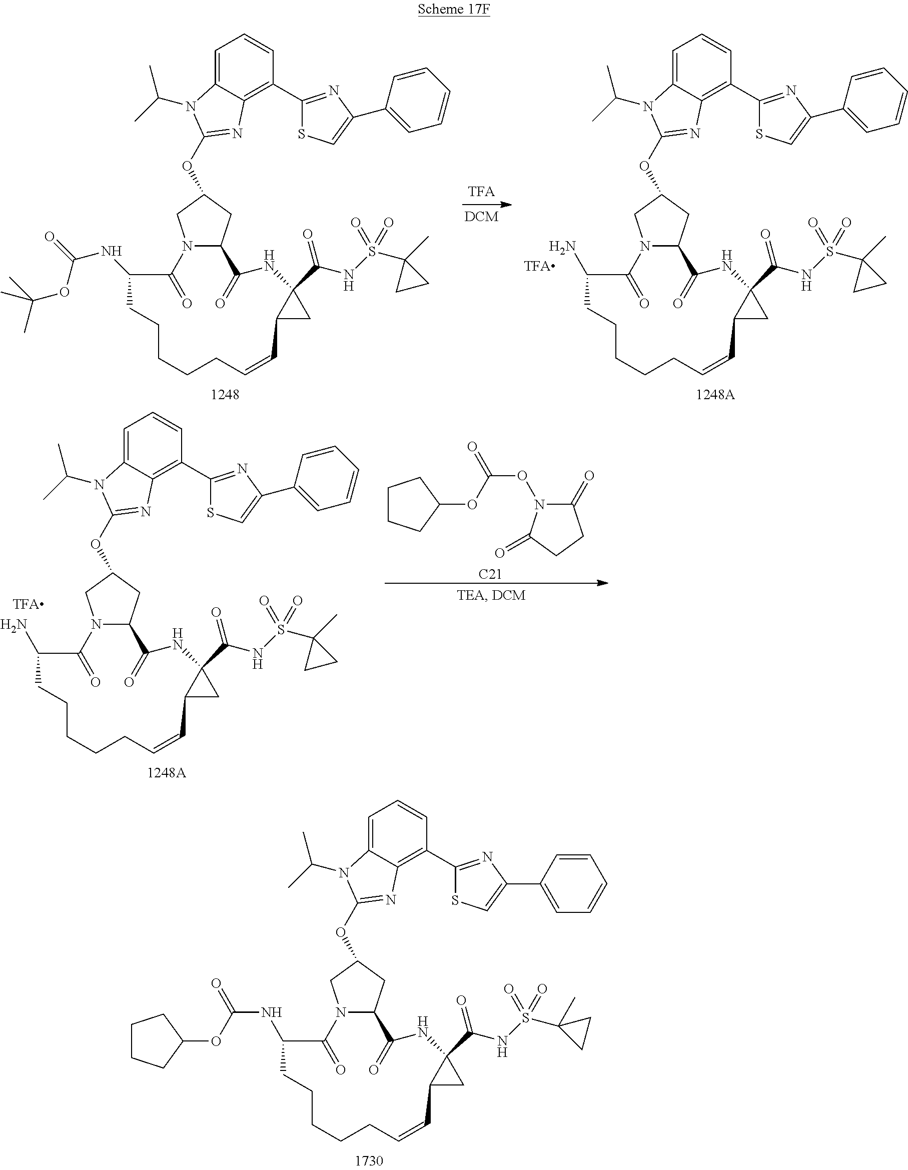

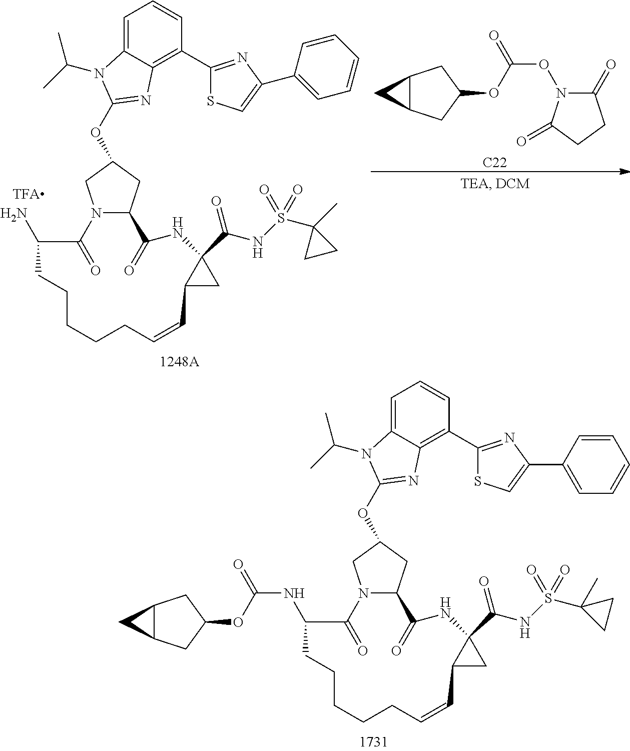

Compound 1248 (1.09 g, 1.2 mmol) was dissolved in DCM (10 mL) and TFA (3 mL). The mixture was stirred at r.t. for 4 h. The resulting mixture was stirred at room temperature for 3 hrs. The reaction was monitored by LCMS. After completion of the reaction, the solvent was removed under reduced pressure to afford compound 1248A, which was used directly in the next step without purification.

A flask was charged with compound 1248A (250 mg, 0.28 mmol), compound C21 (106 mg, 0.468 mmol), TEA (94 mg, 0.94 mmol) and anhydrous DCM (4 mL). The resulting mixture was stirred at room temperature for 2.5 hrs. The reaction was monitored by LCMS. After completion of the reaction, the solvent was removed under reduced pressure. The residue was purified with prep-TLC (PE/EA=1/2) to afford compound 1730 (115.3 mg, yield 45%). MS (ESI) m/z [M+H]⁺ 912.2.

The same procedure was adopted for preparation of compound 1731. (56.3 mg, yield 28%). MS (ESI) m/z [M+H]⁺ 924.2.

A flask was charged with compound 1248A (200 mg, 0.22 mmol), compound C23 (60 mg, 0.44 mmol), TEA (90 mg, 0.88 mmol) and anhydrous DCM (4 mL). The resulting mixture was stirred at room temperature for 3 hrs. The reaction was monitored by LCMS. After completion of the reaction, the solvent was removed under reduced pressure. The residue was purified with prep-HPLC to afford compound 1732 (85 mg, yield 43%). MS (ESI) m/z [M+H]⁺ 897.2.

TABLE 24
Compounds 1730-1732.

| Compound | Structure | Yield |
|---|---|---|
| 1730 | 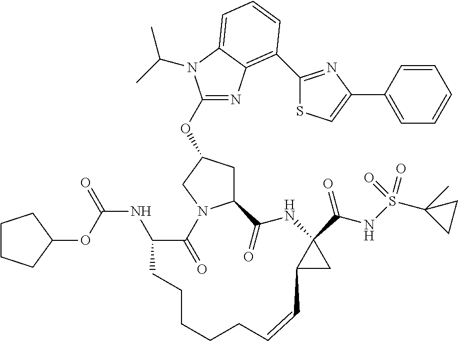 | (115.3 mg, yield 45%). MS (ESI) m/z [M + H]⁺ 912.2. |
| 1731 | 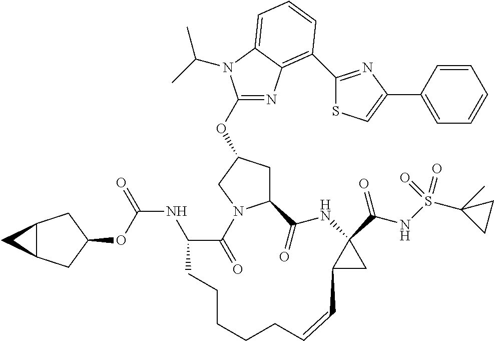 | (56.3 mg, yield 28%). MS (ESI) m/z [M + H]⁺ 924.2. |
| 1732 | 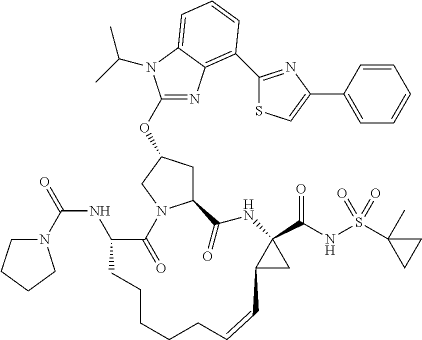 | (85 mg, yield 43%). MS (ESI) m/z [M + H]⁺ 897.2 |

### 17.7 Synthesis of Compound 1733

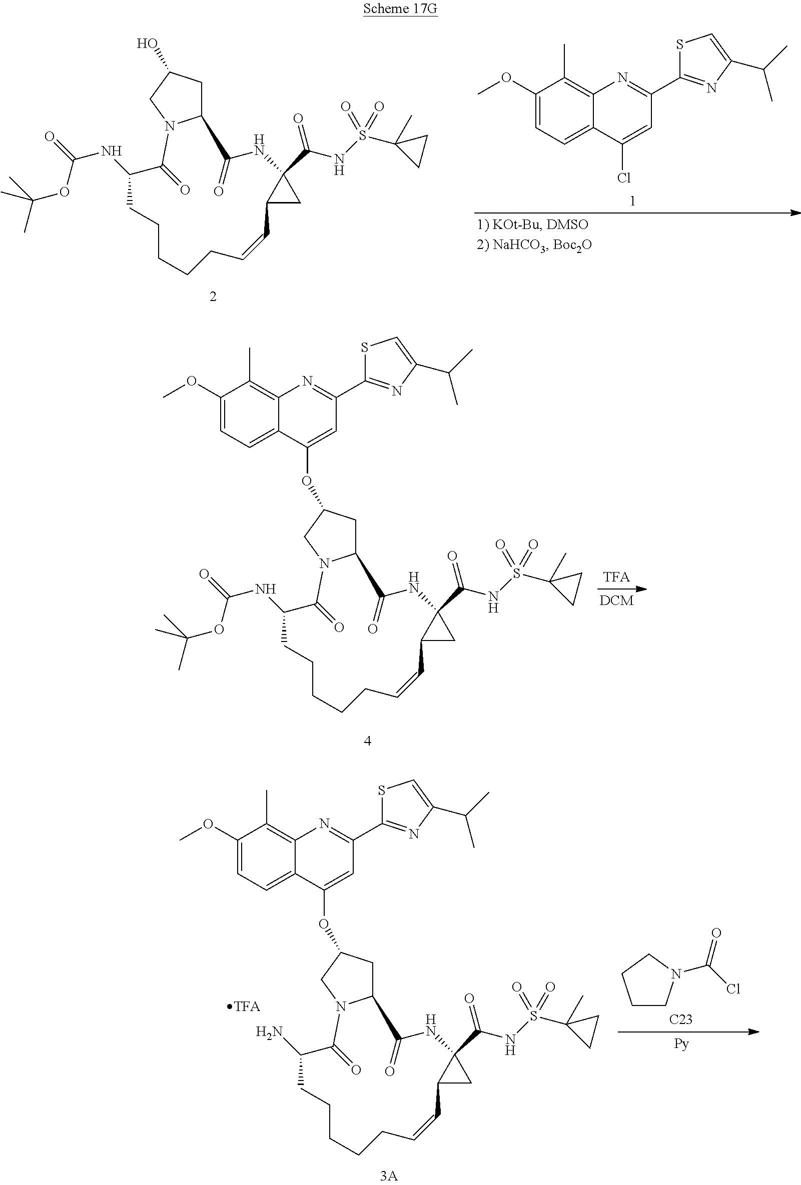

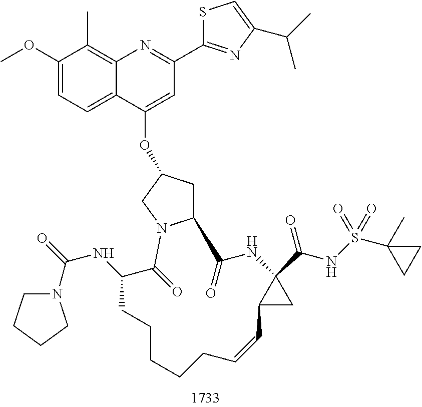

Compound 4 was prepared following Scheme 1A (162 mg, yield 62%). Compound 3A was also prepared following Scheme 1A (60 mg, yield 100%).

To a solution of compound 3A (40 mg, 0.045 mmol, 1 eq.) in 1 mL of pyridine was added compound C23 (8 mg, 0.054, 1.2 eq.) at 0° C. The solution was stirred for 2 hrs at 0° C., then allowed to warm to room temperature, and stirring was continued for another 18 hrs. LCMS analysis showed the reaction completed. The reaction mixture was diluted with ethyl acetate, washed with aq. HCl (1 N), saturated aqueous NaHCO₃ and water. The combined organic layers were dried over anhydrous sodium sulfate, filtered and the solvent was removed under reduced pressure. The residue was purified by prep-TLC to afford compound 1733 (13.9 mg, yield 31%). MS (ESI) m/z (M+H)⁺ 876.4.

### 17.8 Synthesis of Compound 1734

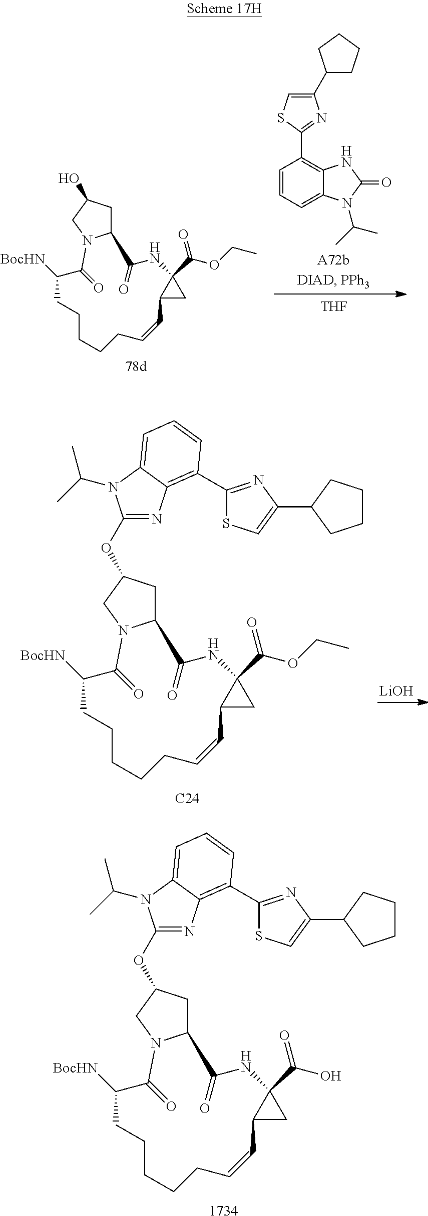

Macrocycle 78d (150 mg, 0.3 mmol), A72b (104 mg, 0.3 mmol), triphenylphosphine (365 mg, 1.5 mmol) and anhydrous tetrahydrofuran (20 mL) were charged into a 100 mL of three neck flask. The reaction mixture was cooled using an ice bath and diisopropylazodicarboxylate (DIAD, 0.3 mL, 1.5 mmol) was added dropwise. The cooling bath was removed and stirring was continued at ambient temperature for an additional 3 hours. Saturated aqueous sodium hydrogen carbonate (10 mL) was added to the stirring mixture and then the mixture was stirred for a further 5 minutes. The mixture was then extracted with DCM. The organic layers were combined and concentrated in vacuo. The residue was purified by prep-TLC affording the desired compound C24 (80 mg, yield 33%) as a brown oil. MS (ESI) m/z (M+H)⁺ 803.5.

A solution of intermediate C24 (80 mg, 0.1 mmol) in dioxane (4 mL) was cooled for 5 minutes using an ice bath. The stirring mixture was treated dropwise with aqueous lithium hydroxide (1 N, 1 mL, 1 mmol) and stirring was continued maintaining the temperature using an ice bath. After addition, the reaction mixture was heated to 40° C. overnight. Progress of the reaction monitored by LCMS. The mixture was treated with citric acid to until pH=5, the mixture was then extracted with EtOAc. The organic extracts were combined, washed with brine and then dried in vacuo to afford compound 1734 (67 mg, yield 87%) as white solid. MS (ESI) m/z (M+Na)⁺ 775.3.

### 17.9 Synthesis of Compounds 1735-1737

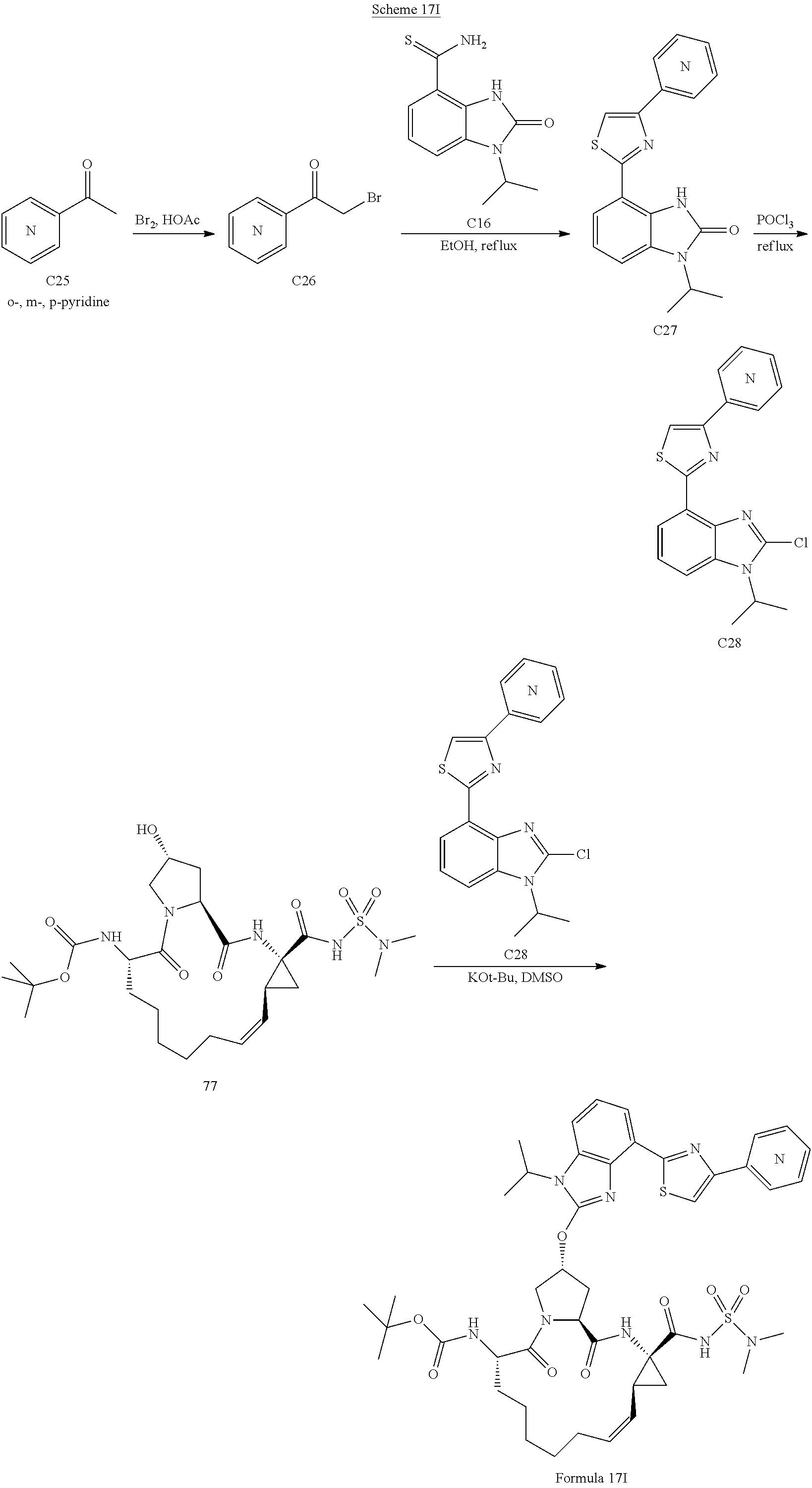

To a solution of HBr/HOAc (14 mL) was added compound C25 (1.0 g, 8.3 mmol) and then dropwise Br₂ (1.45 g, 9.09 mmol). The resulting mixture was heated at 70° C. for 3 hrs. After completion of the reaction, the mixture was cooled to r.t. and poured into hexane and filtered to afford compound C26 as a yellow solid. Compounds C26a-C26C were prepared using this method.

TABLE 25
Compounds C26a-C26c,

| Compound | Structure | Yield |
|---|---|---|
| C26a | 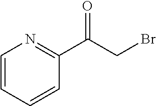 | 1.1 g, yield 67%. Yellow solid. MS (ESI) m/z (M + H)⁺ 199.7. ¹H NMR (400 MHz, DMSO-d₆) δ: 8.71 (d, 1H), 8.019-7.973 (m, 2H), 7.66 (d, 1H), 4.99 (s, 2H). |
| C26b | 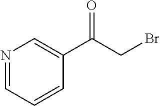 | 1 g, yield 60%. Yellow solid. MS (ESI) m/z (M)⁺ 199.7. ¹H NMR (400 MHz, DMSO-d₆) δ: 9.27 (s, 1H), 8.98 (t, 1H), 8.51 (d, 1H), 7.76 (d, 1H), 5.06 (s, 2H). |
| C26c | 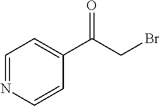 | 1.0 g, yield 60%. Yellow solid. MS (ESI) m/z (M)⁺ 199.6. ¹H NMR (400 MHz, DMSO-d₆) δ: 8.81 (d, 2H), 8.73 (d, 2H), 5.00 (s, 2H). |

To a solution of compound C16 (126 mg, 0.53 mmol) in EtOH (5 mL) was added compound C26 (460 mg, 2.27 mmol). The mixture was refluxed under nitrogen for 1 h. After completion of the reaction, the solvent was evaporated under reduced pressure to give a crude product, which was purified by prep-TLC to afford compound C27. Compounds C27a-C27c were prepared using this method.

TABLE 26
Compounds C27a-C27c.

| Compound | Structure | Yield |
|---|---|---|
| C27a | 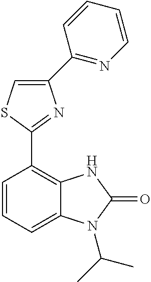 | 110 mg, yield 62%. Light yellow solid. MS (ESI) m/z (M + H)⁺ 336.9 |
| C27b | 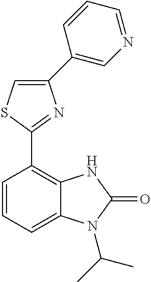 | 115 mg, yield 64%. Light yellow solid. MS (ESI) m/z (M + H)⁺ 336.9 |
| C27c | 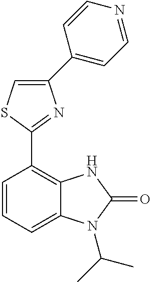 | 189 mg, yield 72.4%. Light yellow solid. MS (ESI) m/z (M + H)⁺ 337 |

General compound C27 was dissolved in POCl₃ (2 mL) and the resulting mixture was refluxed under nitrogen. After completion of reaction, excess of POCl₃ was removed, and then the mixture was taken up with ice-water and neutralized with NaHCO₃ under cooling. The mixture was extracted with EtOAc (30 mL×3). The combined organic layers were washed with brine, dried over anhydrous Na₂SO₄ and concentrated to give a crude product general compound C28, which was used directly in next step. MS (ESI) m/z (M)⁺355.

To a solution of compound 77 (110 mg, 0.193 mmol) in DMSO (4 mL) was added KOt-Bu (200 mg, 1.76 mmol), the mixture was stirred at 0° C. for 1 h under nitrogen. Subsequently, general compound C28 (70.0 mg, 0.193 mmol) was added into the stirring solution, the mixture was stirred at r.t for 1.5 h and then treated with ice water. The mixture was neutralized by addition of aq. HCl (1 M) and the resulting mixture extracted with EtOAc (30 mL×3). The combined organic layers were washed with brine, dried over anhydrous Na₂SO₄, concentrated in vacuo, and purified with prep-HPLC. Compounds 1735-1737 were prepared using this method.

TABLE 27
Compounds 1735-1737.

| Compound | Structure | Yield |
|---|---|---|
| 1735 | 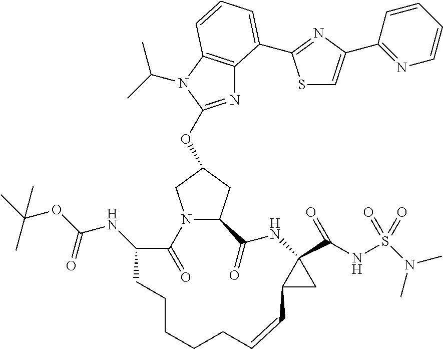 | 50.1 mg, yield 29%. MS (ESI) m/z (M + H)⁺ 890.3. |
| 1736 | 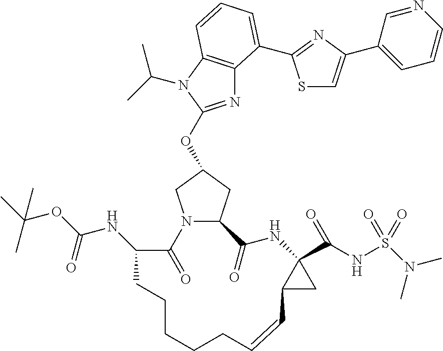 | 30.5 mg, yield 18%. MS (ESI) m/z (M + H)⁺ 890.4. |
| 1737 | 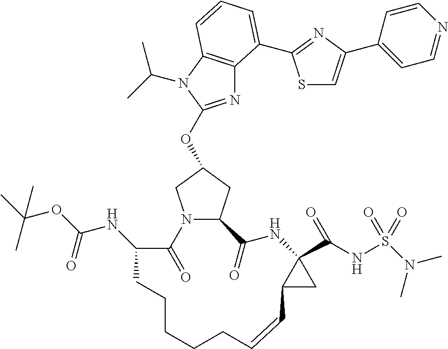 | 70.0 mg, yield 40.9%. MS (ESI) m/z [M + H]⁺ 890.3. |

### 17.10 Synthesis of Compounds 1738-1744

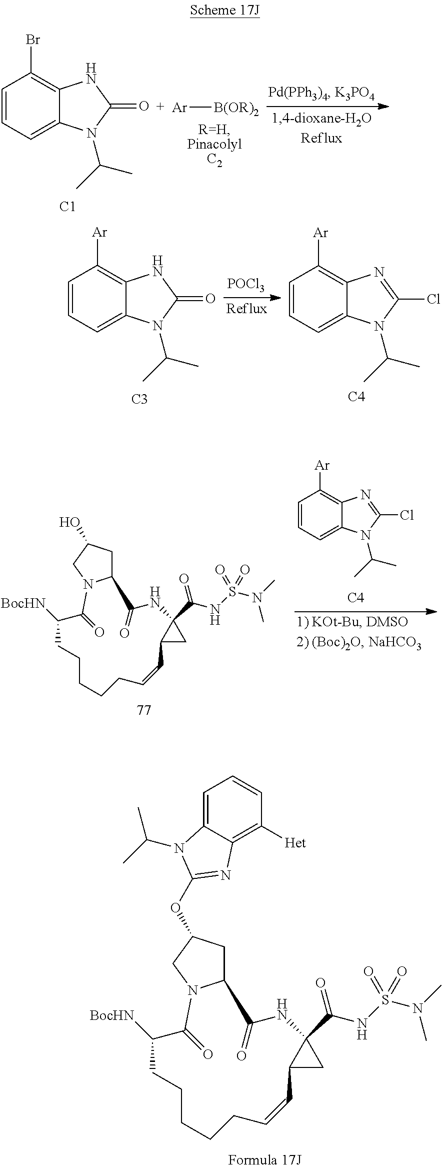

The preparation of compounds C3 and C4 followed the procedure described in section 17.1 (Scheme 17A). The procedure is generally applicable to borates or boronic acids including, but not limited to, those shown below. The following borates were coupled with compound C1 to form various C3:

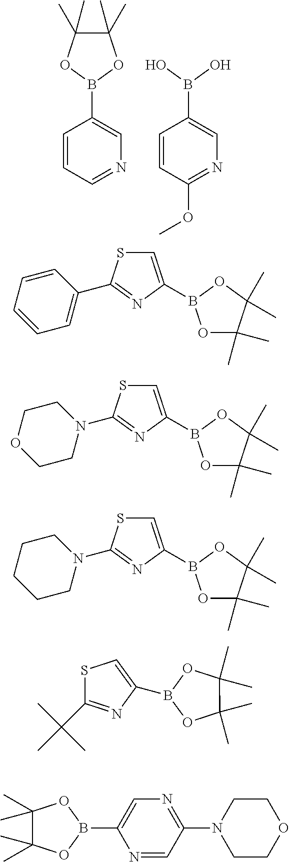

The preparation of formula 17J followed the same procedure described in section 17.1, using compound 77. Compounds 1738-1744 were prepared.

TABLE 28
Compounds 1738-1744.

| Compound | Structure | Yield |
|---|---|---|
| 1738 | 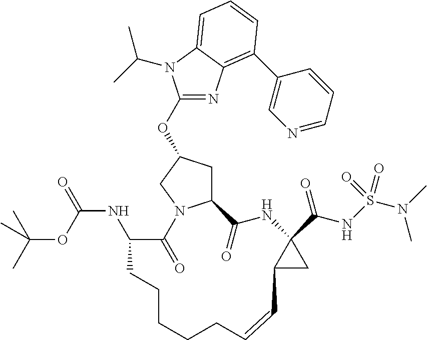 | 22.7 mg, yield 39.8%. MS (ESI) m/z (M + H)⁺ 807.4 |
| 1739 | 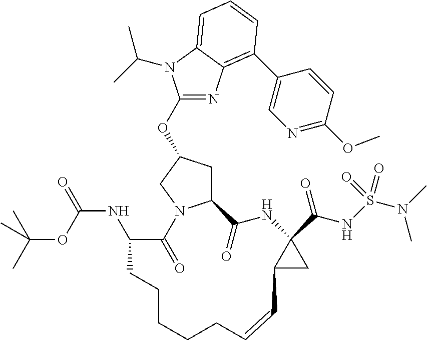 | 27.4 mg, yield 40.2%. MS (ESI) m/z (M + H)⁺ 837.4 |
| 1740 | 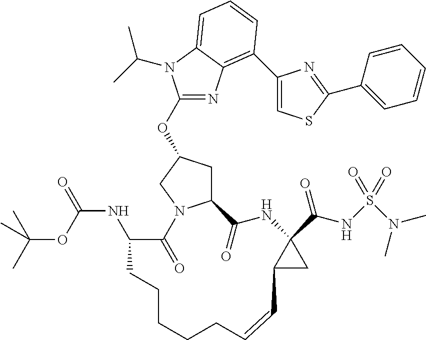 | 13.3 mg, yield 39%. MS (ESI) m/z (M + H)⁺ 889.5 |
| 1741 | 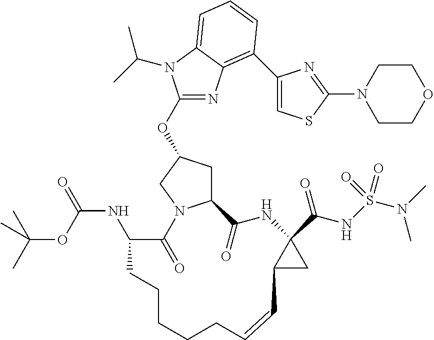 | 9.8 mg, yield 17.5%. MS (ESI) m/z (M + H)⁺ 898.4 |
| 1742 | 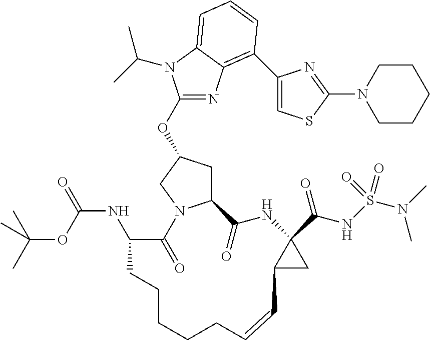 | 7.7 mg, yield 14%. MS (ESI) m/z (M + H)⁺ 896.4 |
| 1743 | 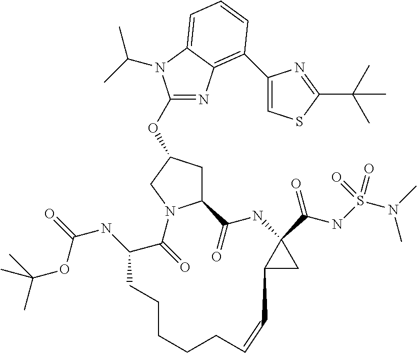 | 8.2 mg, yield 20%. MS (ESI) m/z (M + H)⁺ 869.4 |
| 1744 | 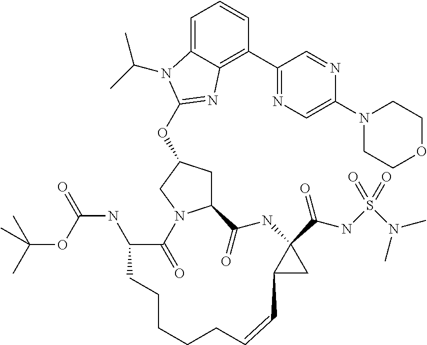 | 11.2 mg, yield 32%. MS (ESI) m/z (M + H)⁺ 893.4 |

### 17.11 Synthesis of Compound 1745

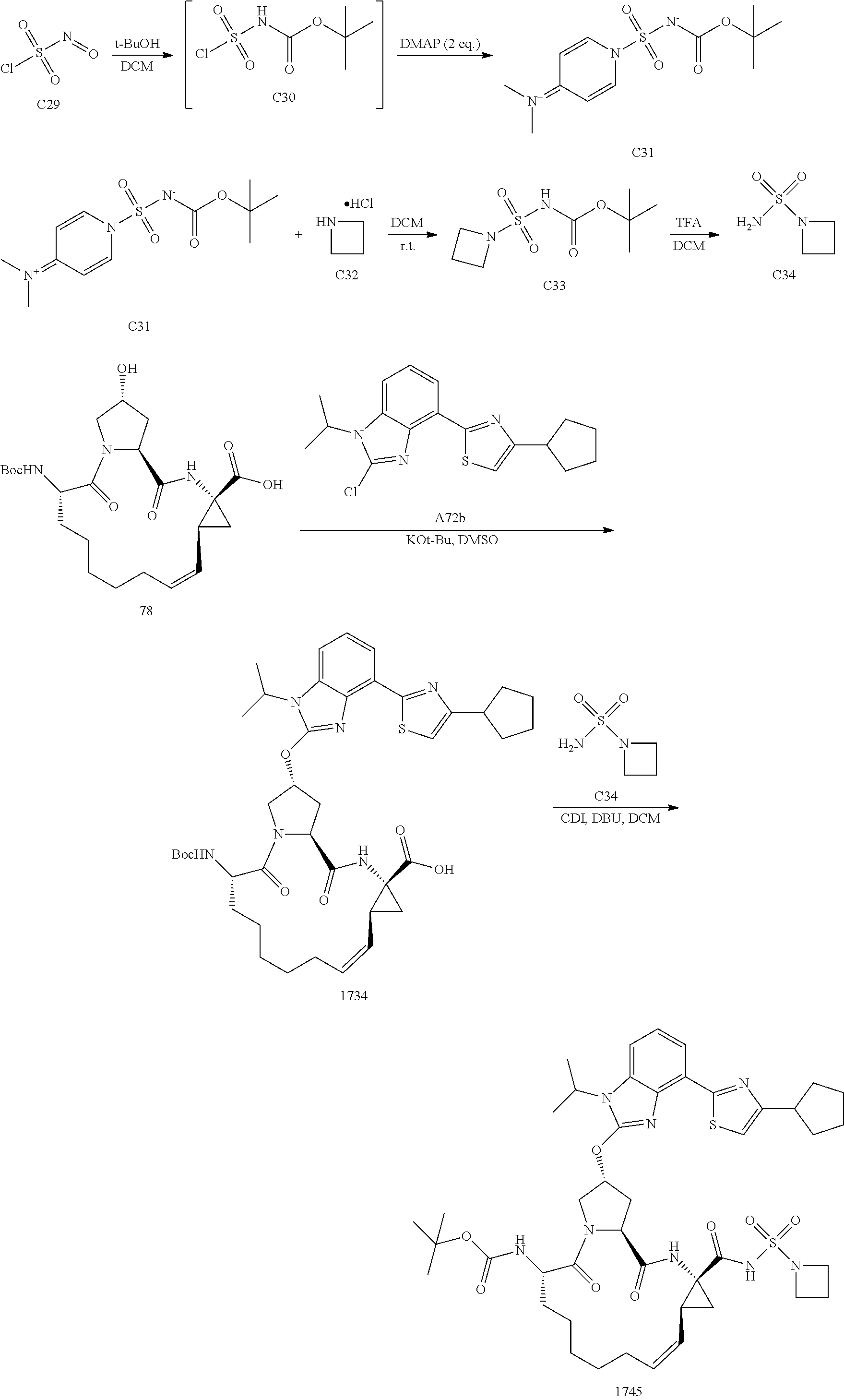

Chlorosulfonyl isocyanate C29 (CSI, 2.4 mL, 28.25 mmol) was added dropwise to a cold solution of t-BuOH (2.1 g, 28.25 mmol) in anhydrous CH₂Cl₂ (20 mL). Subsequently, DMAP (6.9 g, 56.5 mmol) was added. The mixture was stirred for 1 hour at r.t. and then washed several times with water. The organic layer was dried over anhydrous sodium sulfate, and concentrated in vacuo. The colorless powder C31 was used in next step without further purification (5 g, yield 60%).

To a mixture of azetidine HCl salt C32 (260 mg, 2.8 mmol) in anhydrous CH₂Cl₂ (10 mL) was added TEA (280 mg, 2.8 mmol), followed by addition of the sulfamoylating reagent C31 (850 mg, 2.8 mmol) to afford a mixture containing solids. After stirring for 5 min, the solid gradually dissolved to give a clear and almost colorless solution. The mixture was stirred at r.t. overnight. After 17 hrs, TLC indicated completion of the reaction (CH₂Cl₂/MeOH=9/1). The mixture was concentrated in vacuo to afford a residue. The residue was purified by column chromatography on silica gel (eluent: CH₂Cl₂/MeOH=20/1 to 10/1) to afford compound C33 as a white solid (470 mg, yield 71%).

Compound C33 (1 g, 4.2 mmol) was dissolved in TFA/DCM (V/V=1/1, 15 mL) and stirred at r.t. for 2 hrs. The reaction mixture was evaporated in vacuo to give a yellow residue. The residue was treated with diethyl ether and a white solid precipitated. The solid C34 was collected by filtration and the white powder was used directly without further purification (470 mg, 82%). ¹H NMR (400 MHz, Acetone-d₆) δ 2.13-2.20 (m, 2H), 3.78 (t, 4H), 6.07 (br s, 2H).

The intermediate 1734 was prepared as described previously. A solution of compound 1734 (140 mg, 0.18 mmol) and CDI (117 mg, 0.72 mmol) in anhydrous DCM (25 mL) was stirred under a nitrogen atmosphere at reflux for 4 hours and then cyclopropylsulfonamide C34 (98 mg, 0.72 mmol) and DBU (219 mg, 0.88 mmol) was added. The resulting mixture was stirred at reflux overnight. The reaction solution was concentrated, extracted with EtOAc and washed with brine. The organic phase was collected and concentrated in vacuo. The final compound 1745 was purified by prep-HPLC as a light yellow solid (63 mg, 39%), MS (ESI) m/z (M+H)⁺ 893.4.

### 17.12 Synthesis of Compound 1746-1754

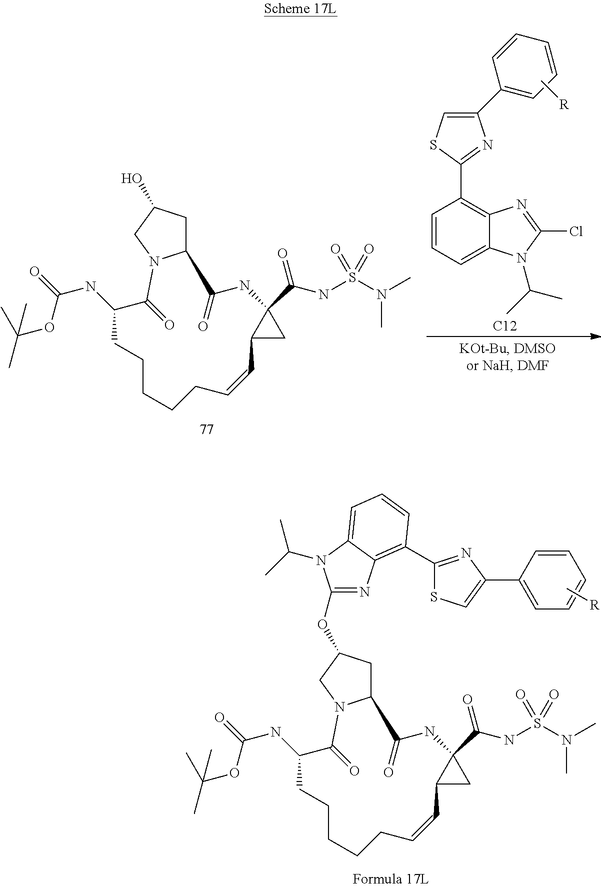

The preparation of formula 17L followed Scheme 17C. Compounds 1746-1747 were prepared via this method using KOt-Bu and DMSO.

TABLE 29
Compounds 1746-1747.

| Compound | Structure | Yield |
|---|---|---|
| 1746 | 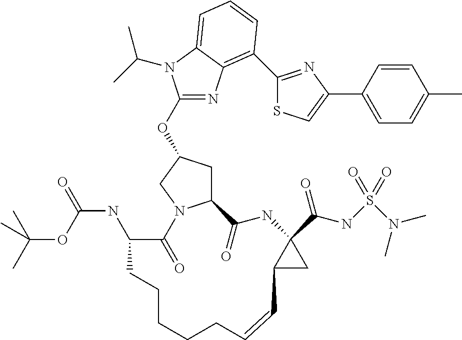 | 9.3 mg, yield 15%. MS (ESI) m/z (M + H)⁺ 903.2 |
| 1747 | 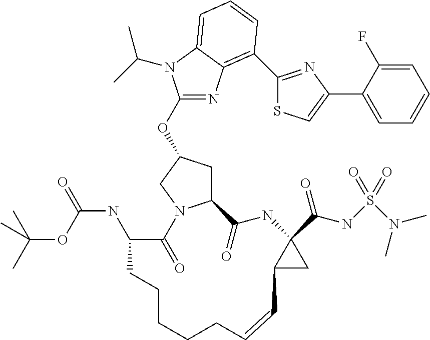 | 15 mg, yield 24%. MS (ESI) m/z (M + H)⁺ 907.3 |

Compounds 1748-1754 were prepared following the same procedure as described for the synthesis of Compound 207 using NaH and DMF.

TABLE 30
Compounds 1748-1754.

| Compound | Structure | Yield |
|---|---|---|
| 1748 | 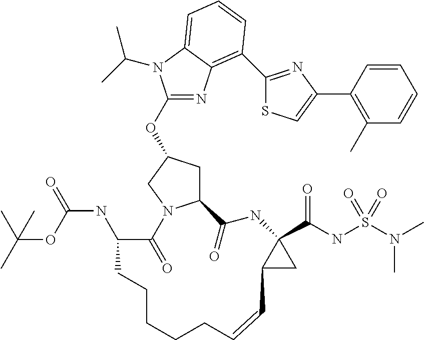 | 14 mg, yield 22%. MS (ESI) m/z (M + H)⁺ 903.2 |
| 1749 | 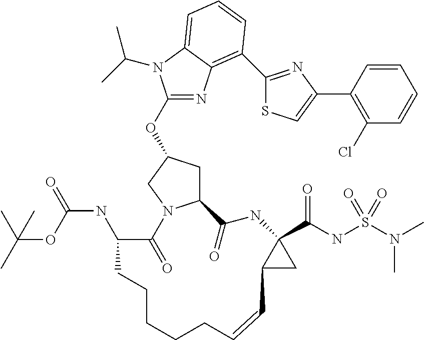 | 16 mg, yield 27%. MS (ESI) m/z (M + H)⁺ 923.3 |
| 1750 | 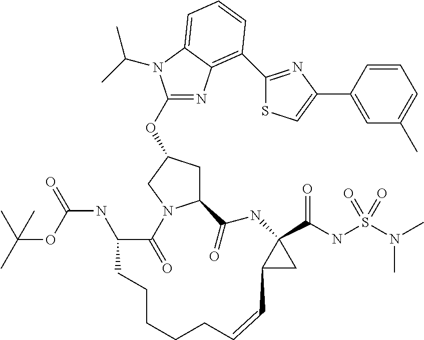 | 10 mg, yield 13%. MS (ESI) m/z (M + H)⁺ 903.2 |
| 1751 | 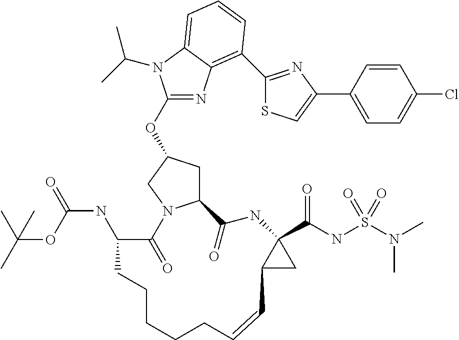 | 15.3 mg, yield 20%. MS (ESI) m/z (M + H)⁺ 923.2 |
| 1752 | 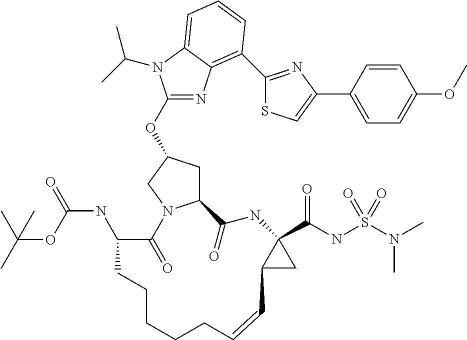 | 11.6 mg, yield 15%. MS (ESI) m/z (M + H)⁺ 919.2 |
| 1753 | 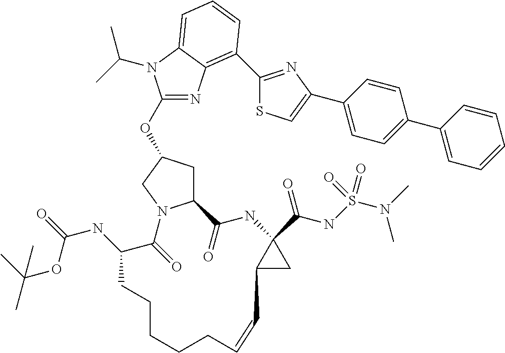 | 16 mg, yield 19%. MS (ESI) m/z (M + H)⁺ 965.2 |
| 1754 | 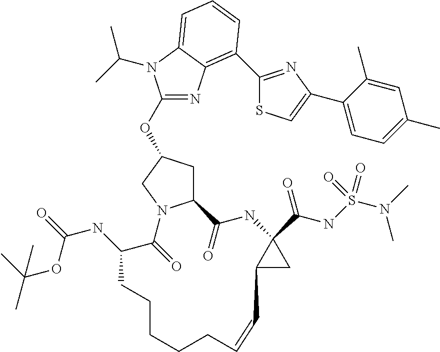 | 16 mg, yield 20%. MS (ESI) m/z (M + H)⁺ 917.3 |

### 17.13 Synthesis of Compound 1755-1761

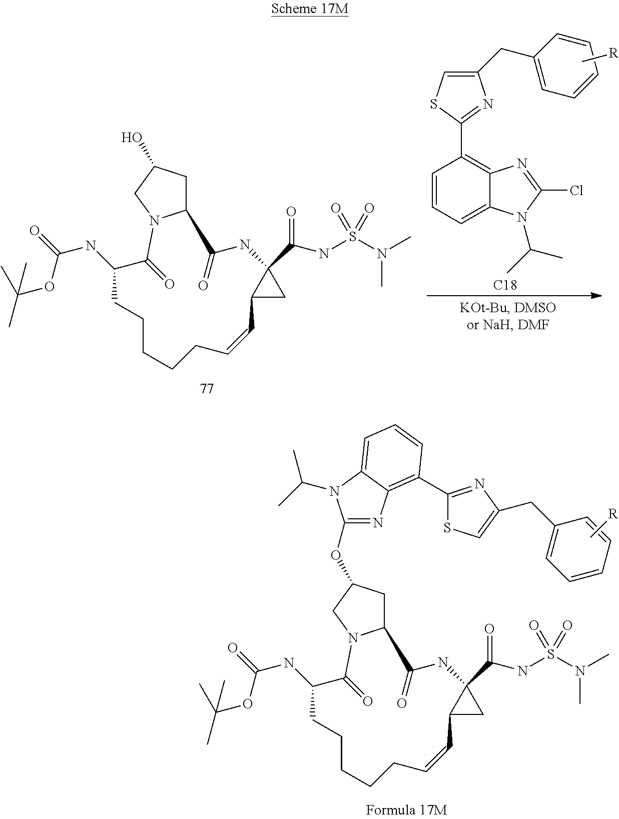

The preparation of formula 17M followed Scheme 17C. KOt-Bu and DMSO was used for the preparation of compounds 1755-1757.

TABLE 31
Compounds 1755-1757.

| Compound | Structure | Yield |
|---|---|---|
| 1755 | 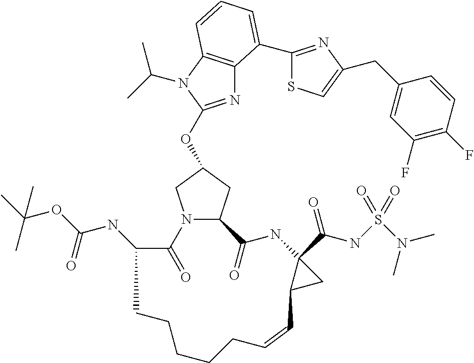 | 20 mg, yield 25%. MS (ESI) m/z (M + H)⁺ 939.3 |
| 1756 | 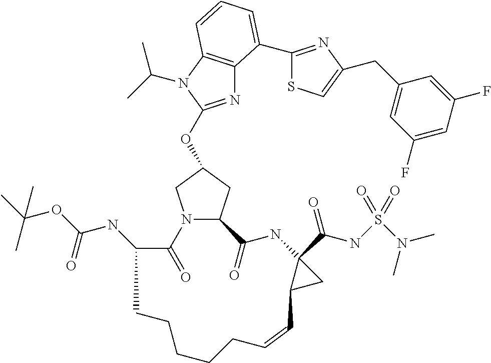 | 20 mg, yield 25%. MS (ESI) m/z (M + H)⁺ 939.3 |
| 1757 | 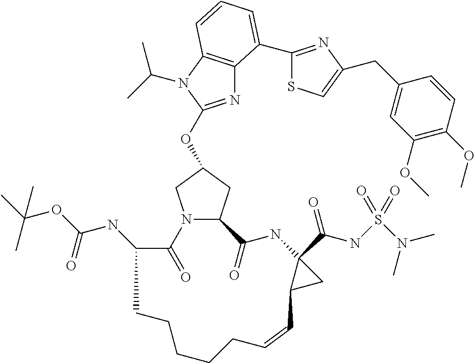 | 12.2 mg, yield 15%. MS (ESI) m/z (M + H)⁺ 963.2 |

Similar to the synthesis of Compound 207, NaH and DMF was used for the preparation of compounds 1758-1761.

TABLE 32
Compounds 1758-1761.

| Compound | Structure | Yield |
|---|---|---|
| 1758 | 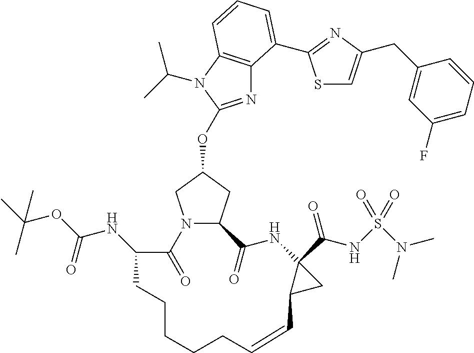 | 10.9 mg, yield 14%. MS (ESI) m/z (M + H)⁺ 921.3 |
| 1759 | 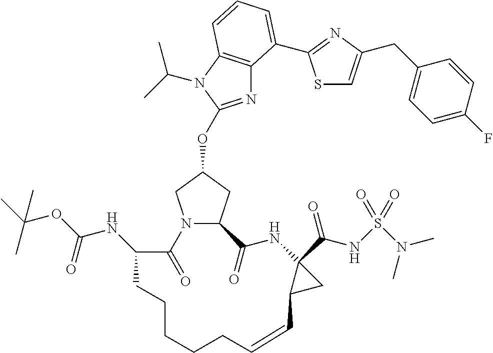 | 16.4 mg, yield 21%. MS (ESI) m/z (M + H)⁺ 921.3 |
| 1760 | 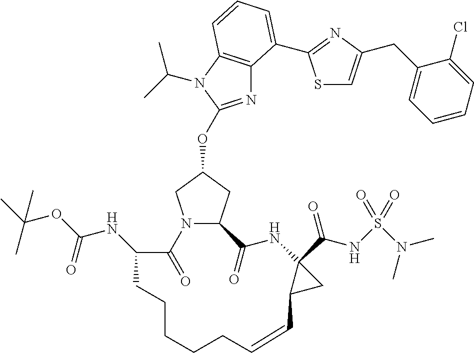 | 21.6 mg, yield 26%. MS (ESI) m/z (M + H)⁺ 937.3 |
| 1761 | 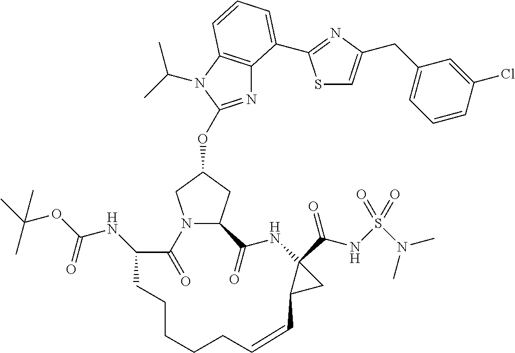 | 17.8 mg, yield 22%. MS (ESI) m/z (M + H)⁺ 937.3 |

### 17.14 Synthesis of Compound 1762-1763

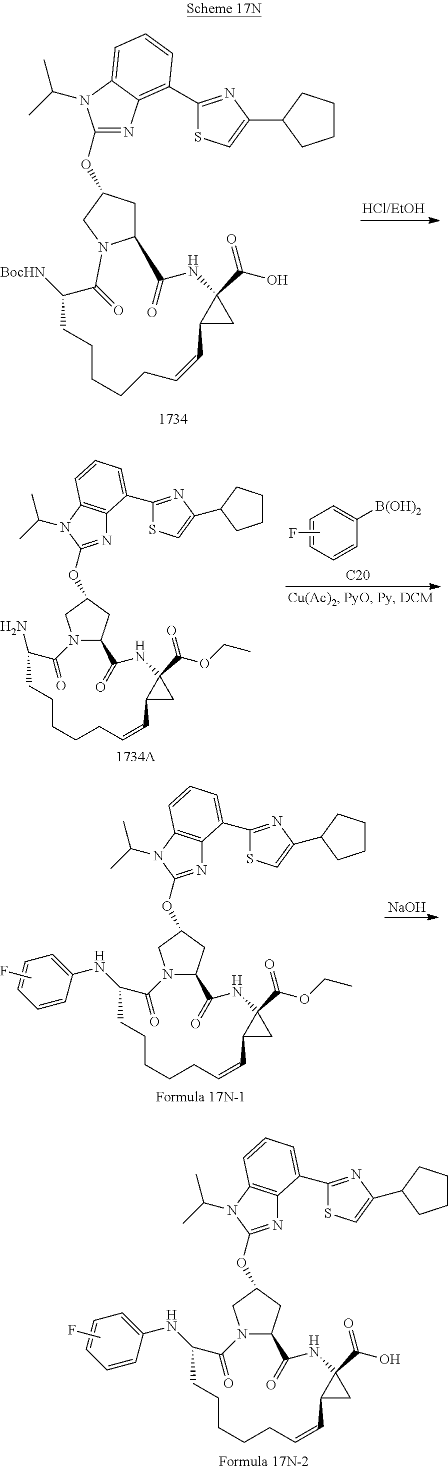

To a solution of compound 1734 (600 mg, 0.77 mmol) in HCl/Dioxane (12 mL) was added EtOH (5 mL), the resulting solution was stirred at ambient temperature for 2 days. Subsequently, the solution was evaporated in vacuo to give compound 1734A as a colorless solid. Compound 1734A was used directly in the next step. MS (ESI) m/z (M+H)⁺703.3.

A mixture of compound 1734A (250 mg, 0.35 mmol.), general compound C20 (149 mg, 1 mmol.), Cu(OAc)₂ (181 mg, 1 mmol.), pyridine (276 mg, 3.5 mmol.), pyridine N-oxide (340 mg, 3.5 mmol.) and molecular sieves (4A) in dichloromethane (15 mL) was stirred for 2 days at room temperature under an atmosphere of oxygen. The reaction was monitored by LCMS. After completion of the reaction, the mixture was diluted with ethyl acetate (50 mL) and filtered. The filtrate was washed with brine, dried over anhydrous sodium sulfate, and concentrated in vacuo. The residue was purified with prep-TLC to afford formula 17N-1. Compound 17N-1a and 17N-1b were prepared.

TABLE 33
Compounds 17N-1a-17N-1b.

| Compound | Structure | Yield |
|---|---|---|
| 17N-1a | 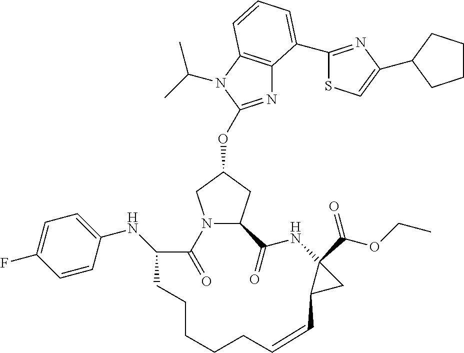 | 120 mg, yield 42%. MS (ESI) m/z (M + H)⁺ 797.5. |
| 17N-1b | 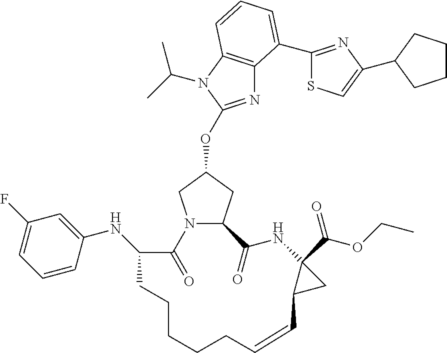 | 140 mg, yield 49%. MS (ESI) m/z (M + H)⁺ 797.4 |

NaOH (10 eq.) was added to a solution of a compound of formula 17N-1 in MeOH and water (v/v=5:1). The resulting solution was stirred at ambient temperature overnight. The mixture was concentrated and treated with aq. HCl (1 M) until reaching a pH=5˜6. The resulting mixture was extracted with EtOAc (50 mL×3). The combined organic layers were washed with brine, dried over anhydrous Na₂SO₄ and concentrated in vacuo. The final compound of formula 17N-2 was purified by prep-TLC. Compounds 1762-1763 were prepared.

TABLE 34
Compounds 1762-1763.

| Compound | Structure | Yield |
|---|---|---|
| 1762 | 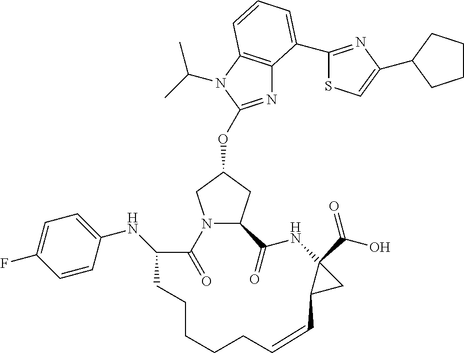 | 121.7 mg, yield 19%. MS (ESI) m/z (M + H)⁺ 769.3 |
| 1763 | 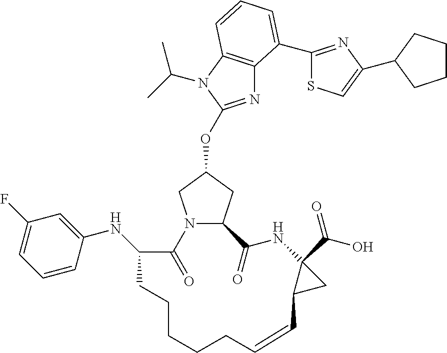 | 31.2 mg, yield 23%. MS (ESI) m/z (M + H)⁺ 769.3 |

### 17.15 Synthesis of Compounds 1764-1778

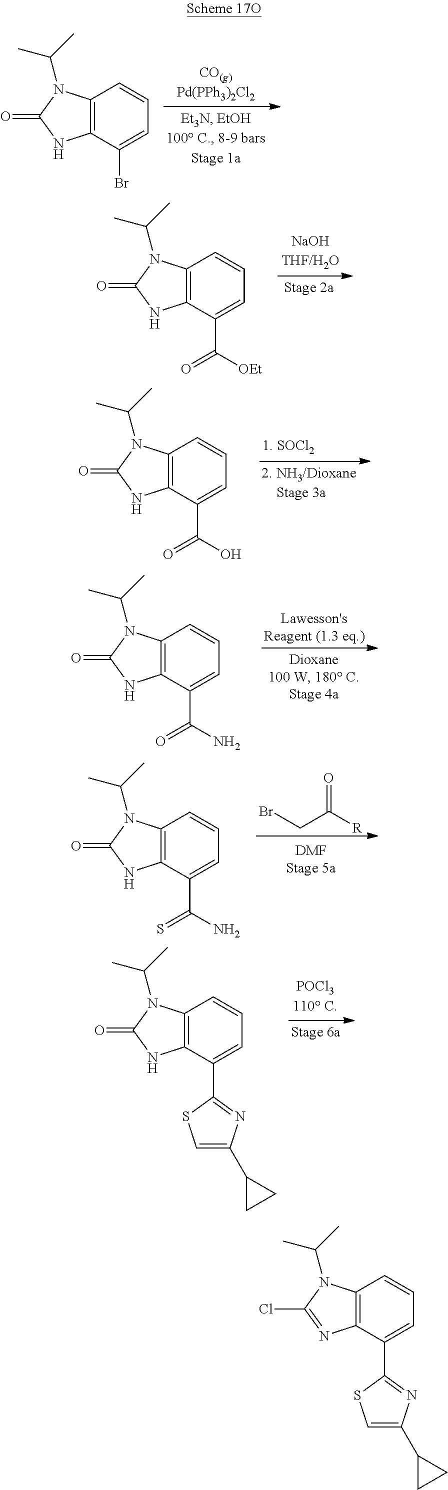

Stage 1a: 1-isopropyl-2-oxo-4-bromo-benzimidazole (32.6 g, 0.123 mmol, 1 eq.), triethylamine (58 g, 0.564 mol, 4.5 eq.) and ethanol (800 mL) were charged into a 2L pressure vessel. Bis(triphenylphosphine)palladium dichloride (4.4 g, 6.26 mmol, 5 mol %) was added and the reaction mixture heated at 100° C. under 10 bar of carbon monoxide for 15 hours. The reaction mixture was left to cool down to room temperature and the pressure released. Checking the reaction by LCMS showed a 25% conversion ratio. The reaction mixture was filtered to remove a small amount of black solid. Fresh catalyst (5 mol %) and triethylamine (50 mL) were added and the reaction mixture was heated at 100° C. under 8.5 bars of carbon monoxide for another 15 hours. LCMS analysis of an aliquot showed the conversion has reached 74%. The reaction mixture was filtered to remove extra black solid. Fresh catalyst (5 mol %) was added and the reaction mixture was heated at 100° C. under 8.5 bars of carbon monoxide for a further 15 hours. LCMS analysis of an aliquot showed the reaction to be complete. The solvent was removed in vacuo and the residue diluted with ethyl acetate (300 mL). The organic phase was washed with 1M hydrochloric acid (300 mL), water (300 mL) and brine (300 mL), dried over magnesium sulfate, filtered and the solvent removed in vacuo. The residue was purified by flash column chromatography using an ethyl acetate/heptane gradient. After combining the relevant fractions, the solvent was removed in vacuo to afford 25.6 g (78% yield) of the desired compound as a free flowing pale yellow solid. ¹H NMR (500 MHz, CDCl₃) δ ppm 9.03 (br. s., 1H) 7.65 (dd, J=8.09, 0.92 Hz, 1H) 7.29 (d, J=8.39 Hz, 1H) 7.09 (t, J=8.01 Hz, 1H) 4.74 (spt, J=7.02 Hz, 1H) 4.44 (q, J=7.07 Hz, 2H) 1.55 (d, J=7.02 Hz, 6H) 1.43 (t, J=7.17 Hz, 3H). LC-MS: purity 90% (UV), t_{R} 1.86 min m/z [M+H]⁺ 248.95 (MET/CR/1278).

Stage 2a: Ethyl 1-isopropyl-2-oxo-benzimidazole-4-carboxylate (25.6 g, 0.101 mmol, 1 eq.), water (125 mL) and tetrahydrofuran (250 mL) were charged into a 1 L round bottom flask. Sodium hydroxide (44.9 g, 1.01 mol, 10 eq.) was added portion wise over 5 minutes and the resulting reaction mixture heated at 70° C. for 5 hours by which time no starting material could be detected by LCMS analysis. The biphasic reaction mixture was left to cool down to room temperature and the phases were separated. The aqueous phase was acidified to pH=2 with hydrochloric acid and then extracted twice with an isopropanol/chloroform mixture (3:1, 100 mL). The organic phases were combined, dried over magnesium sulfate, filtered and the solvent removed in vacuo to afford 22.4 g (99% yield) of desired title compound as a pale pink solid. ¹H NMR (500 MHz, CDCl₃) δ ppm 13.09 (br. s., 1H) 10.56 (br. s., 1H) 7.48 (d, J=7.93 Hz, 1H) 7.48 (d, J=7.93 Hz, 1H) 7.06 (t, J=7.93 Hz, 1H) 4.60 (spt, J=6.97 Hz, 1H) 1.44 (d, J=7.02 Hz, 6H). LC-MS: purity 95% (UV), t_{R} 1.54 min m/z [M+H]⁺ 220.95 (MET/CR/1278).

Stage 3a: 1-Isopropyl-2-oxo-benzimidazole-4-carboxylic acid (2.855 g, 12.96 mmol, 1.0 eq.) was charged into a 100 mL round bottom flask and the flask placed on top of an ice bath. Thionyl chloride (28 mL) was added portion wise and the reaction mixture stirred at ambient temperature for 15 hours. Thionyl chloride was removed in vacuo and the residue diluted with dry dioxane (20 mL). Ammonia in dioxane (0.5 M, 39 mL, 19.44 mmol, 1.5 eq) was added dropwise over 10 min. The reaction mixture was then stirred at ambient temperature for 15 hours. The solvent was removed in vacuo and the residue triturated with water (20 mL) and dioxane (5 mL) leading to precipitation of a solid which was collected by filtration. After further drying under high vacuum for 4 hours 3.68 g (94%) of the desired compound was isolated as a beige solid. ¹H NMR (500 MHz, CDCl₃) δ ppm 9.66 (br. s., 1H) 7.26 (d, J=7.78 Hz, 1H) 7.18 (d, J=7.78 Hz, 1H) 7.07 (m, J=7.78, 7.78 Hz, 1H) 6.00 (br. s., 2H) 4.75 (spt, J=6.99 Hz, 1H) 1.55 (d, J=7.02 Hz, 6H). LC-MS: purity 92% (UV), t_{R} 1.38 min m/z [M+H]⁺ 219.90 (MET/CR/1278).

Stage 4a: 1-Isopropyl-2-oxo-benzimidazole-4-carboxylic acid amide (1.67 g, 8.0 mmol, 1.0 eq.) and dioxane (18 mL) were charged into a pressure tube. Lawesson's reagent (2.46 g, 6.0 mmol, 0.8 eq.) was added and the reaction mixture heated at 85° C. for 50 minutes. The reaction mixture was left to cool down to ambient temperature and the solvent removed in vacuo. The residue was purified by flash column chromatography using a ethyl acetate/heptanes gradient to give 1.35 g (51% corrected yield) of the desired product as yellow oil which was used in the next step without further purification. ¹H NMR (500 MHz, CDCl₃) δ ppm 9.66 (br. s., 1H) 7.26 (d, J=7.78 Hz, 1H) 7.18 (d, J=7.78 Hz, 1H) 7.07 (m, J=7.78, 7.78 Hz, 1H) 6.00 (br. s., 2H) 4.75 (spt, J=6.99 Hz, 1H) 1.55 (d, J=7.02 Hz, 6H). LC-MS: purity 68% (UV), t_{R} 1.52 min m/z [M+H]⁺ 235.95 (MET/CR/1278).

Stage 5a: 1-Isopropyl-2-oxo-benzimidazole-4-carbothioic acid amide (200 mg, 0.85 mmol, 1.0 eq.) and N,N-dimethylformamide (2 mL) were charged into a 10 mL vial. 1-Cyclopropyl-2-bromoethanone (138 mg, 0.85 mmol, 1.0 eq.) was added and the reaction mixture stirred at ambient temperature for 15 hours. Saturated aqueous sodium hydrogencarbonate (2 mL) was added and the reaction mixture extracted with ethyl acetate (3×2 mL). The organic extracts were combined, washed with water (2×2 mL), dried over magnesium sulfate, filter and the solvent removed in vacuo to give 125 mg (45% yield) of the desired product as a yellow oil which was used in the next step without any further purification. ¹H NMR (500 MHz, CDCl₃) δ ppm 9.65 (br. s., 1H) 7.38 (d, J=7.78 Hz, 1H) 7.10-7.18 (m, 1H) 7.00-7.10 (m, 1H) 6.81 (s, 1H) 4.70-4.82 (m, 1H) 1.99-2.22 (m, 1 H) 1.56 (d, J=7.02 Hz, 6H) 1.05-1.16 (m, 2H) 0.89-1.04 (m, 2H). LC-MS: purity 92% (UV), t_{R} 2.31 min m/z [M+H]⁺ 299.95 (MET/CR/1278).

Stage 6a: 1-Isopropyl-2-oxo-4-(4-cyclopropyl-thiazol-2-yl)-1,3-dihydro-benzoimidazole (400 mg, 1.0 mmol, 1.0 eq.) and phosphorous oxychloride (4 mL) were heated at 85° C. for 15 hours. The reaction mixture was left to cool down to ambient temperature and the solvent removed in vacuo. Aqueous potassium carbonate (5 mL) was added to the residue then more potassium carbonate solution was added until pH=7. The aqueous phase was extracted with ethyl acetate (3×2 mL). The organic extracts were combined, washed with aqueous potassium carbonate (2 mL), dried over magnesium sulfate, filtered and the solvent removed in vacuo to give 191 mg (40% yield) of the desired product as a brown oil which was used in the next step without any further purification. ¹H NMR (500 MHz, CDCl₃) δ ppm 8.23 (d, J=7.78 Hz, 1H) 7.52 (d, J=8.09 Hz, 1H) 7.34 (t, J=8.01 Hz, 1H) 6.98 (s, 1H) 4.96 (spt, J=6.99 Hz, 1H) 2.13-2.24 (m, 1H) 1.68 (d, J=7.02 Hz, 6 H) 0.93-1.04 (m, 4H). LC-MS: purity 87% (UV), t_{R} 2.52 min m/z [M+H]⁺ 318.00 (MET/CR/1278). Compounds C35a-C35g were prepared.

TABLE 35
Compounds C35a-C35g.

| Compound | Structure | Yield |
|---|---|---|
| 1-isopropyl-4-(5,6- dihydro-4H- cyclohexathiazol-2- yl]-benzimidazole (C35a) | 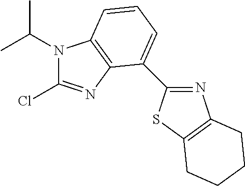 | 94 mg (73% yield) as a brown oil. ¹H NMR (500 MHz, CDCl₃) δ ppm 8.47 (d, J = 7.93 Hz, 1 H) 7.68 (d, J = 8.24 Hz, 1 H) 7.45 (t, J = 8.09 Hz, 1 H) 4.97 (spt, J = 6.97 Hz, 1 H) 3.06 (br. s., 2 H) 2.86 (br. s., 2 H) 1.89-1.98 (m, 4 H) 1.69 (d, J = 7.02 Hz, 6 H). LC-MS: purity 90% (UV), tR 2.55 min m/z [M + H]⁺ 332.35 (MET/CR/1278). |
| 1-isopropyl-4-(5,6- dihydro-4H- cyclopentathiazol-2- yl]-benzimidazole (C35b) | 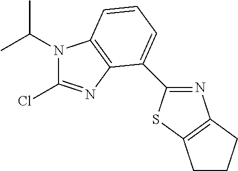 | 68 mg (70% yield) as a brown oil. ¹H NMR (500 MHz, CDCl₃) δ ppm 8.40 (d, J = 7.93 Hz, 1 H) 7.77 (d, J = 8.24 Hz, 1 H) 7.53 (t, J = 7.93 Hz, 1 H) 5.00 (spt, J = 6.97 Hz, 1 H) 3.26 (t, J = 7.32 Hz, 2 H) 3.09 (t, J = 7.32 Hz, 2 H) 2.73 (t, J = 7.32 Hz, 2 H) 1.72 (d, J = 7.02 Hz, 6 H). LC-MS: purity 60% (UV), t_{R} 2.46 min m/z [M + H]⁺ 318.35 (MET/CR/1278). |
| 1-isopropyl-2-chloro- 4-(4- cyclobutylthiazol-2- yl)-benzimidazole (C35c) | 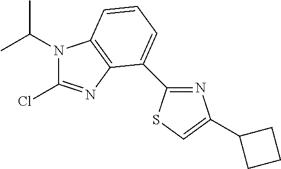 | 169 mg (99% yield) as a brown solid. ¹H NMR (500 MHz, CDCl₃) δ ppm 8.47 (d, J = 7.63 Hz, 1 H) 7.62 (d, J = 7.93 Hz, 1 H) 7.41 (t, J = 7.93 Hz, 1 H) 7.20 (s, 1 H) 4.97 (spt, J = 6.97 Hz, 1 H) 3.96 (quin, J = 8.54 Hz, 1 H) 2.46-2.56 (m, 2 H) 2.25-2.35 (m, 2 H) 2.03-2.16 (m, 1 H) 1.90-1.99 (m, 1 H) 1.68 (d, J = 7.02 Hz, 6 H). LC- MS: purity 89% (UV), t_{R} 2.67 min m/z [M + H]⁺ 332.40 (MET/CR/1278). |
| 1-isopropyl-2-chloro- 4-(4-benzylthiazol-2- yl)-benzimidazole (C35d) | 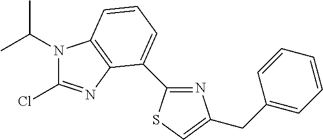 | 399 mg (80% yield) as a brown oil. LC- MS: purity 84% (UV), t_{R} 2.65 min m/z [M + H]⁺ 368.35 (MET/CR/1278). |
| 1-isopropyl-2-chloro- 4-(4- cyclohexylthiazol-2- yl)-benzimidazole (C35e) | 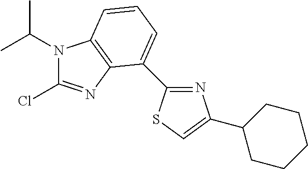 | 366 mg (75% yield) as a brown oil. LC- MS: purity 78% (UV), t_{R} 2.89 min m/z [M + H]⁺ 360.10 (MET/CR/1278). |
| 1-isopropy1-2-chloro- 4-(4- cyclohexylmethylthiazol- 2-yl)-benzimidazole (C35f) | 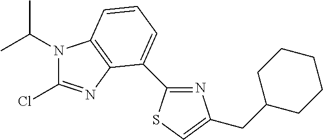 | 208 mg (82% yield) as a brown oil. LC- MS: purity 87% (UV), t_{R} 2.96 min m/z [M + H]⁺ 374.40 (MET/CR/1278). |
| 1-isopropyl-4-(5,6- dihydro-4H- (dimethyl)cyclohexathiazol- 2-yl]- benzimidazole (C35g) | 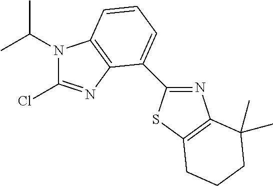 | 435 mg (93% yield) as a brown oil. LC- MS: purity 57% (UV), t_{R} 2.86 min m/z [M + H]⁺ 360.10 (MET/CR/1278) |

Compounds 1764-1774 and 1776-1780 were prepared following the procedure for preparing compound 1201. Compound 1315 was synthesized according to the method for synthesizing Compound 1218.

TABLE 36
Compounds 1755-1757.

| Compound | Structure | Yield |
|---|---|---|
| 1764 | 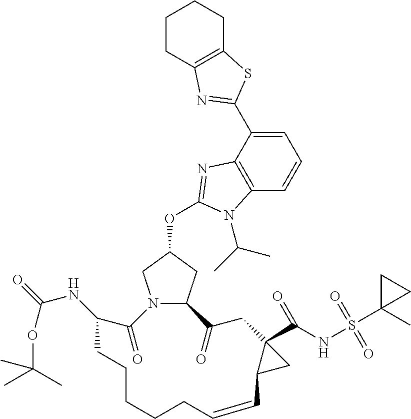 | 44 mg (30% yield) as an off-white solid after preparative HPLC. ¹H NMR (500 MHz, CDCl₃) δ ppm 10.11 (br. s., 1 H) 8.07 (d, J = 7.63 Hz, 1 H) 7.25-7.28 (m, 1 H) 7.20 (t, J = 7.78 Hz, 1 H) 6.77 (br. s., 1 H) 5.97 (br. s., 1 H) 5.74 (q, J = 8.90 Hz, 1 H) 5.06 (d, J = 10.38 Hz, 1 H) 5.03 (t, J = 9.77 Hz, 1 H) 4.56-4.67 (m, 2 H) 4.53 (d, J = 11.90 Hz, 1 H) 4.28-4.36 (m, 1 H) 4.16 (dd, J = 11.67, 3.43 Hz, 1 H) 2.90 (d, J = 5.34 Hz, 4 H) 2.79-2.87 (m, 1 H) 2.69-2.79 (m, 1 H) 2.51-2.61 (m, 1 H) 2.28 (q, J = 8.54 Hz, 1 H) 1.86-1.96 (m, 6 H) 1.75-1.83 (m, 2 H) 1.58-1.63 (m, 1 H) 1.54 (d, J = 6.87 Hz, 6 H) 1.50-1.53 (m, 2 H) 1.50 (s, 3 H) 1.44- 1.48 (m, 2 H) 1.38-1.44 (m, 1 H) 1.35 (s, 9 H) 1.27-1.33 (m, 2 H) 1.13-1.23 (m, 1 H) 0.79-0.86 (m, 2 H). LC-MS: purity 99% (UV), t_{R} 4.71 min m/z [M + H]⁺ 878.20 (MET/CR/1426). HRMS: Found: 878.3940, calculated for C₄₄H₅₉N₇O₈S₂ (M + H)⁺: 878.3945. |
| 1765 | 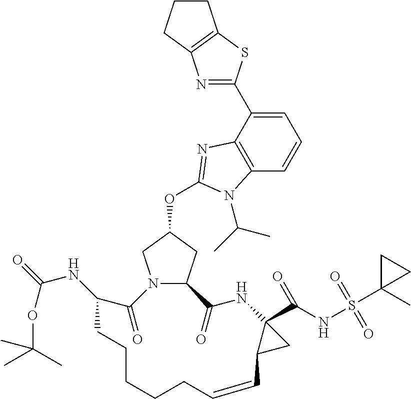 | 8.2 mg (5.6% yield) as a white solid after preparative HPLC. ¹H NMR (500 MHz, CDCl₃) δ ppm 10.13 (br. s., 1 H) 8.08 (d, J = 7.63 Hz, 1 H) 7.27 (d, J = 7.63 Hz, 1 H) 7.21 (m, J = 7.78, 7.78 Hz, 1 H) 6.77 (s, 1 H) 5.96 (br. s., 1 H) 5.74 (q, J = 9.00 Hz, 1 H) 4.98-5.09 (m, 2 H) 4.55-4.67 (m, 2 H) 4.52 (d, J = 11.75 Hz, 1 H) 4.28-4.37 (m, 1 H) 4.20 (dd, J = 11.44, 3.66 Hz, 1 H) 2.98- 3.08 (m, 2 H) 2.96 (t, J = 7.25 Hz, 2 H) 2.80- 2.89 (m, 1 H) 2.70-2.79 (m, 1 H) 2.50- 2.61 (m, 3 H) 2.27 (q, J = 8.44 Hz, 1 H) 1.86- 1.97 (m, 2 H) 1.74-1.86 (m, 2 H) 1.58- 1.63 (m, 1 H) 1.54 (d, J = 6.87 Hz, 6 H) 1.50- 1.53 (m, 1 H) 1.50 (s, 3 H) 1.44-1.49 (m, 3 H) 1.39-1.44 (m, 1 H) 1.36 (s, 9 H) 1.24- 1.33 (m, 2 H) 1.14-1.24 (m, 1 H) 0.79- 0.87 (m, 2 H). LC-MS: purity 100% (UV), t_{R} 4.79 min m/z [M + H]⁺ 864.20 (MET/CR/1426). HRMS: Found: 864.3799, calculated for C₄₃H₅₇N₇O₈S₂ (M + H)⁺: 864.3788. |
| 1766 | 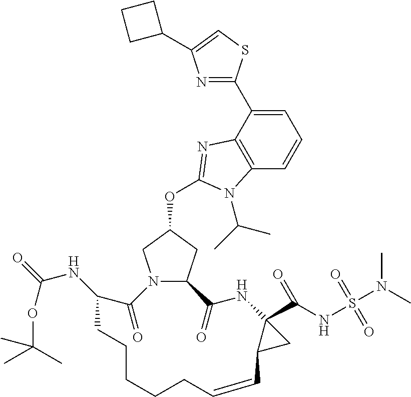 | 128 mg (35% yield) as a beige solid after preparative HPLC. ¹H NMR (500 MHz, CDCl₃) δ ppm 10.02 (br. s., 1 H) 8.15 (d, J = 7.63 Hz, 1 H) 7.29 (d, J = 7.93 Hz, 1 H) 7.22 (t, J = 7.93 Hz, 1 H) 7.08 (s, 1 H) 6.70 (br. s., 1 H) 5.93-6.00 (m, 1 H) 5.76 (q, J = 9.00 Hz, 1 H) 4.99-5.09 (m, 2 H) 4.55- 4.67 (m, 2 H) 4.52 (d, J = 11.44 Hz, 1 H) 4.27-4.35 (m, 1 H) 4.16 (dd, J = 11.75, 3.97 Hz, 1 H) 3.79 (quin, J = 8.54 Hz, 1 H) 2.89 (s, 6 H) 2.73-2.87 (m, 2 H) 2.52-2.66 (m, 1 H) 2.39-2.48 (m, 2 H) 2.29-2.39 (m, 2 H) 2.22 (q, J = 8.34 Hz, 1 H) 2.00-2.12 (m, 1 H) 1.84-2.00 (m, 3 H) 1.73-1.84 (m, 1 H) 1.55 (d, J = 6.87 Hz, 6 H) 1.39-1.52 (m, 5H) 1.36 (s, 9 H) 1.25-1.34 (m, 2 H) 1.13- 1.23 (m, 1 H). LC-MS: purity 100% (UV), t_{R} 4.94 min m/z [M + H]⁺ 867.25 (MET/CR/1426). HRMS: Found: 867.3909, calculated for C₄₂H₅₈N₈O₈S₂ (M + H)⁺: 867.3897. |
| 1767 | 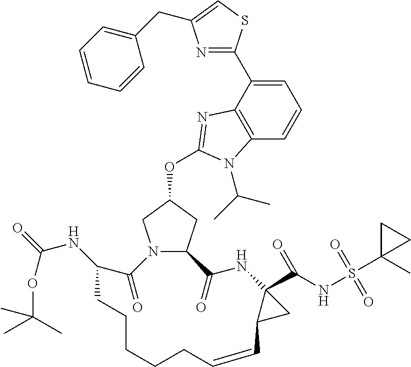 | 132 mg (37% yield) as a cream solid after preparative HPLC. ¹H NMR (500 MHz, CDCl₃) δ ppm 10.06 (br. s., 1 H) 8.15 (d, J = 7.78 Hz, 1 H) 7.31-7.39 (m, 4 H) 7.28- 7.31 (m, 1 H) 7.18-7.26 (m, 2 H) 6.90 (s, 1 H) 6.65 (br. s., 1 H) 5.96 (br. s., 1 H) 5.73 (q, J = 8.95 Hz, 1 H) 4.92-5.09 (m, 2 H) 4.56-4.69 (m, 2 H) 4.46-4.56 (m, 1H) 4.28-4.35 (m, 1 H) 4.26 (s, 2 H) 4.08- 4.18 (m, 1 H) 2.72-2.90 (m, 2 H) 2.47- 2.63 (m, 1 H) 2.26 (q, J = 8.60 Hz, 1 H) 1.85- 1.98 (m, 2 H) 1.72-1.85 (m, 2 H) 1.59- 1.69 (m, 1 H) 1.55 (d, J = 6.87 Hz, 6 H) 1.49 (s, 3 H) 1.40-1.52 (m, 5 H) 1.36 (br. s., 9 H) 1.23-1.34 (m, 2 H) 1.05-1.24 (m, 1 H) 0.82 (br. s., 2 H). LC-MS: purity 100% (UV), t_{R} 5.00 min m/z [M + H]⁺ 914.20 (MET/CR/1426). |
| 1768 | 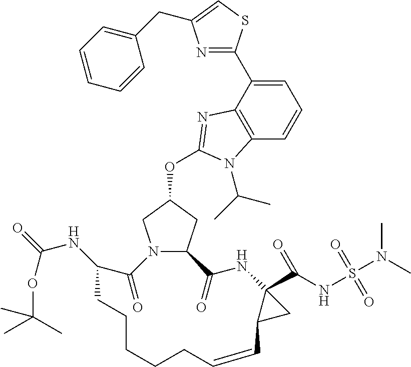 | 126 mg (35% yield) as a cream solid after preparative HPLC. ¹H NMR (500 MHz, CDCl₃) δ ppm 9.96 (br. s., 1 H) 8.13-8.18 (m, 1 H) 7.31-7.38 (m, 4 H) 7.28-7.31 (m, 1 H) 7.18-7.26 (m, 2 H) 6.91 (s, 1 H) 6.60 (br. s., 1 H) 5.89-5.99 (m, 1 H) 5.67- 5.81 (m, 1 H) 4.94-5.09 (m, 2 H) 4.47- 4.67 (m, 3 H) 4.27-4.34 (m, 1 H) 4.26 (s, 2 H) 4.07-4.19 (m, 1 H) 2.89 (s, 6 H) 2.72- 2.86 (m, 2 H) 2.50-2.63 (m, 1 H) 2.22 (q, J = 8.70 Hz, 1 H) 1.84-1.96 (m, 2 H) 1.71- 1.83 (m, 1 H) 1.58-1.68 (m, 1 H) 1.53- 1.56 (m, 6 H) 1.39-1.52 (m, 5 H) 1.36 (s, 9 H) 1.21-1.34 (m, 2 H). LC-MS: purity 100% (UV), t_{R} 4.95 min m/z [M + H]⁺ 903.55 (MET/CR/1426). HRMS: Found: 903.3890, calculated for C₄₅H₅₈N₈O₈S₂ (M + H)⁺: 903.3897. |
| 1769 | 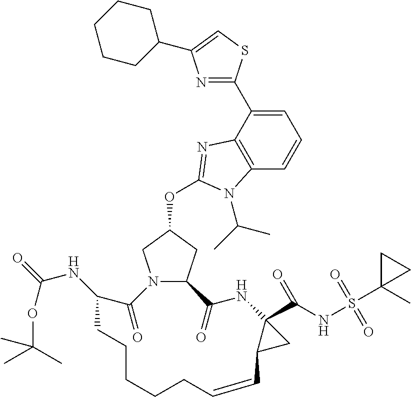 | 74 mg (21% yield) as an off-white solid after preparative HPLC. ¹H NMR (500 MHz, CDCl₃) δ ppm 10.07 (br. s., 1 H) 8.14 (d, J = 7.72 Hz, 1 H) 7.28-7.31 (m, 1 H) 7.18- 7.24 (m, 1 H) 7.00 (s, 1 H) 6.66 (br. s., 1 H) 5.98 (br. s., 1 H) 5.74 (q, J = 8.93 Hz, 1 H) 4.96-5.09 (m, 2 H) 4.56-4.68 (m, 2 H) 4.52 (d, J = 13.40 Hz, 1 H) 4.27-4.37 (m, 1 H) 4.10-4.22 (m, 1 H) 2.85-2.94 (m, 1 H) 2.74-2.85 (m, 2 H) 2.49-2.61 (m, 1 H) 2.26 (q, J = 8.88 Hz, 1 H) 2.20 (d, J = 11.66 Hz, 2 H) 1.90-1.99 (m, 2 H) 1.83-1.89 (m, 2 H) 1.79 (br. s., 2 H) 1.60-1.70 (m, 1 H) 1.53-1.57 (m, 1 H) 1.55 (d, J = 6.78 Hz, 6 H) 1.50 (s, 3 H) 1.50 (br. s., 9 H) 1.36 (br. s., 9 H) 1.24-1.34 (m, 3 H) 1.06-1.26 (m, 1 H) 0.76-0.87 (m, 2 H). LC-MS: purity 100% (UV), t_{R} 5.25 min m/z [M + H]⁺ 906.35 (MET/CR/1426). HRMS: Found: 906.4272, calculated for C₄₆H₆₃N₇O₈S₂ (M + H)⁺: 906.4258. |
| 1770 | 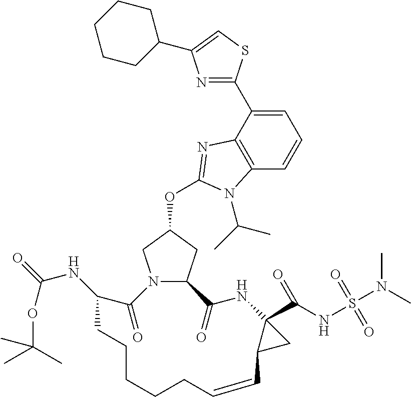 | 91 mg (26% yield) as an off-white solid after preparative HPLC. ¹H NMR (500 MHz, CDCl₃) δ ppm 9.99 (br. s., 1 H) 8.16 (d, J = 7.57 Hz, 1 H) 7.29-7.32 (m, 1 H) 7.19- 7.26 (m, 1 H) 7.02 (s, 1 H) 6.65 (br. s., 1 H) 5.94-6.02 (m, 1 H) 5.77 (q, J = 8.99 Hz, 1 H) 4.97-5.12 (m, 2 H) 4.56-4.69 (m, 2 H) 4.53 (d, J = 11.51 Hz, 1 H) 4.25-4.37 (m, 1 H) 4.11-4.23 (m, 1 H) 2.91 (s, 6 H) 2.86- 2.94 (m, 1 H) 2.73-2.86 (m, 2 H) 2.52- 2.65 (m, 1 H) 2.16-2.29 (m, 3 H) 1.84- 1.97 (m, 4 H) 1.73-1.83 (m, 2 H) 1.62- 1.70 (m, 1 H) 1.56 (d, J = 6.94 Hz, 6 H) 1.40- 1.54 (m, 8 H) 1.38 (br. s., 9 H) 1.33 (br. s., 3 H) 1.11-1.26 (m, 1 H). LC-MS: purity 99% (UV), t_{R} 5.19 min m/z [M + H]⁺ 895.85 (MET/CR/1426). HRMS: Found: 895.4191, calculated for C₄₄H₆₂N₈O₈S₂ (M + H)⁺: 895.4210. |
| 1771 | 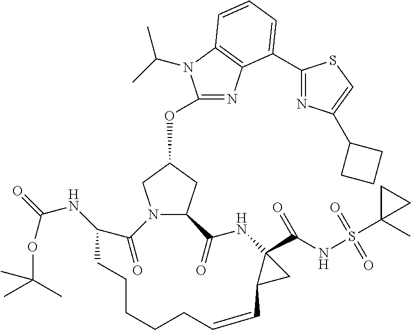 | 104 mg (30% yield) as an off-white solid after preparative HPLC. ¹H NMR (500 MHz, CDCl₃) δ ppm 10.08 (br. s., 1 H) 8.16 (d, J = 7.17 Hz, 1 H) 7.28-7.31 (m, 1 H) 7.18-7.24 (m, 1 H) 7.07 (s, 1 H) 6.67 (br. s., 1 H) 5.98 (br. s., 1 H) 5.74 (q, J = 8.90 Hz, 1 H) 4.99-5.07 (m, 1 H) 4.55-4.67 (m, 2 H) 4.52 (d, J = 12.21 Hz, 1 H) 4.32 (ddd, J = 10.49, 7.74, 3.13 Hz, 1 H) 4.12- 4.22 (m, 1 H) 3.70-3.84 (m, 1 H) 2.72- 2.90 (m, 2 H) 2.50-2.62 (m, 1 H) 2.30- 2.48 (m, 3 H) 2.26 (q, J = 8.70 Hz, 1 H) 2.01- 2.12 (m, 1 H) 1.87-2.01 (m, 3 H) 1.74- 1.86 (m, 2 H) 1.60-1.70 (m, 1 H) 1.55 (d, J = 7.02 Hz, 6 H) 1.50 (s, 3 H) 1.40-1.53 (m, 7 H) 1.36 (s, 9 H) 1.25-1.34 (m, 2 H) 1.10-1.25 (m, 1 H) 0.72-0.88 (m, 2 H). LC-MS: purity 100% (UV), t_{R} 5.01 min m/z [M + H]⁺ 878.55 (MET/CR/1426). HRMS: Found: 878.3951, calculated for C₄₄H₅₉N₇O₈S₂ (M + H)⁺: 878.3945. |
| 1772 | 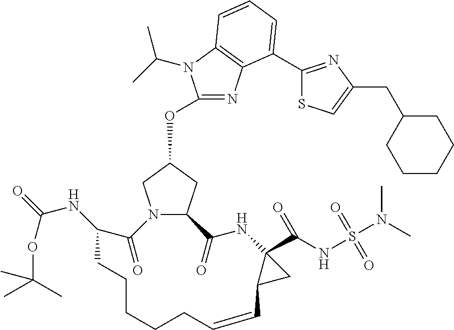 | 47 mg (28% yield) as a white solid after preparative HPLC. ¹H NMR (500 MHz, CDCl₃) δ ppm 9.98 (br. s., 1 H) 8.15 (d, J = 7.72 Hz, 1 H) 7.28-7.31 (m, 1 H) 7.18- 7.25 (m, 1 H) 7.00 (s, 1 H) 6.61 (br. s., 1 H) 5.97 (br. s., 1 H) 5.76 (q, J = 9.35 Hz, 1 H) 4.94-5.08 (m, 2 H) 4.55-4.69 (m, 2 H) 4.52 (d, J = 12.14 Hz, 1 H) 4.23-4.36 (m, 1 H) 4.06-4.22 (m, 1 H) 2.90 (s, 6 H) 2.77- 2.85 (m, 2 H) 2.75 (d, J = 6.94 Hz, 2 H) 2.53- 2.64 (m, 1 H) 2.22 (q, J = 8.35 Hz, 1 H) 1.87-1.97 (m, 2 H) 1.75-1.87 (m, 3 H) 1.60-1.75 (m, 4 H) 1.55 (d, J = 7.09 Hz, 6 H) 1.40-1.53 (m, 5 H) 1.37 (s, 9 H) 1.12- 1.34 (m, 6 H) 0.94-1.09 (m, 2 H). LC-MS: purity 100% (UV), t_{R} 5.24 min m/z [M + H]⁺ 909.20 (MET/CR/1426). HRMS: Found: 909.4349, calculated for C₄₅H₆₄N₈O₈S₂ (M + H)⁺: 909.4367. |
| 1773 | 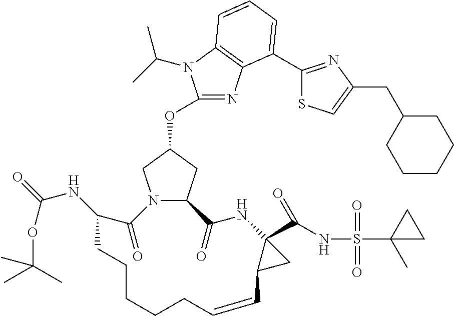 | 61 mg (33% yield) as a pale yellow solid after preparative HPLC. ¹H NMR (500 MHz, CDCl₃) δ ppm 10.08 (br. s., 1 H) 8.14 (d, 1 H) 7.28-7.31 (m, 1 H) 7.19-7.24 (m, 1 H) 7.00 (s, 1 H) 6.65 (br. s., 1 H) 5.98 (br. s., 1 H) 5.68-5.79 (m, 1 H) 4.97-5.09 (m, 2 H) 4.55-4.68 (m, 2 H) 4.48-4.55 (m, 1 H) 4.27-4.37 (m, 1 H) 4.10-4.22 (m, 1H) 2.77-2.90 (m, 2 H) 2.75 (d, J = 6.87 Hz, 2 H) 2.50-2.62 (m, 1 H) 2.26 (q, J = 9.05 Hz, 1 H) 1.88-1.98 (m, 2 H) 1.74-1.87 (m, 5 H) 1.60-1.74 (m, 5 H) 1.55 (d, J = 6.87 Hz, 6 H) 1.51 (s, 3 H) 1.42-1.54 (m, 4 H) 1.37 (s, 9 H) 1.12-1.34 (m, 6 H) 0.94-1.07 (m, 2 H) 0.79-0.87 (m, 2 H). LC-MS: purity 100% (UV), t_{R} 5.32 min m/z [M + H]⁺ 920.30 (MET/CR/1426). HRMS: Found: 920.4430, calculated for C₄₇H₆₅N₇O₈S₂ (M + H)⁺: 920.4414. |
| 1774 | 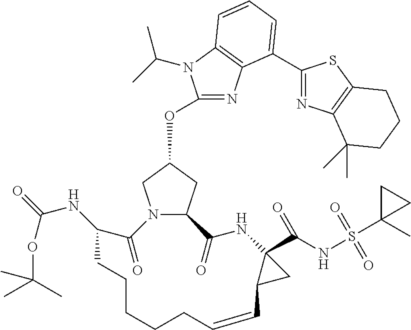 | 75 mg (23% yield) as a pale yellow solid after preparative HPLC. ¹H NMR (500 MHz, CDCl₃) δ ppm 9.76-10.58 (m, 1 H) 8.11 (d, J = 7.48 Hz, 1 H) 7.22-7.26 (m, 1 H) 7.16-7.22 (m, 1 H) 6.72 (br. s., 1 H) 5.96 (br. s., 1 H) 5.73 (q, J = 9.05 Hz, 1 H) 4.98-5.11 (m, 2 H) 4.55-4.66 (m, 2 H) 4.45-4.54 (m, 1 H) 4.33 (br. s., 1 H) 4.11- 4.22 (m, 1 H) 2.79-2.92 (m, 3 H) 2.70- 2.79 (m, 1 H) 2.50-2.61 (m, 1 H) 2.28 (q, J = 8.75 Hz, 1 H) 1.87-1.98 (m, 4 H) 1.77- 1.86 (m, 2 H) 1.70-1.76 (m, 2 H) 1.63- 1.68 (m, 1 H) 1.53 (d, J = 6.87 Hz, 6 H) 1.50 (s, 3 H) 1.42-1.49 (m, 5 H) 1.38-1.42 (m, 6 H) 1.36 (br. s., 9 H) 1.25-1.33 (m, 3 H) 0.76-0.89 (m, 2 H). LC-MS: purity 97% (UV), t_{R} 5.15 min m/z [M + H]⁺ 906.35 (MET/CR/1426). HRMS: Found: 906.426, calculated for C₄₆H₆₃N₇O₈S₂ (M + H)⁺: 906.4258. |
| 1775 | 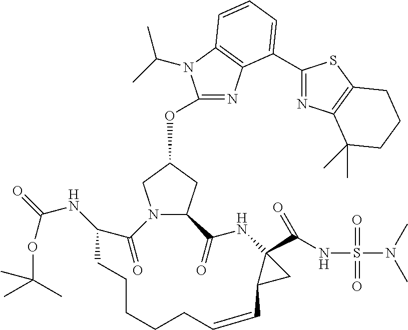 | 80 mg (27% yield) as a pale yellow solid after preparative HPLC. ¹H NMR (500 MHz, CDCl₃) δ ppm 9.82-10.28 (m, 1 H) 8.11 (d, J = 7.48 Hz, 1 H) 7.22-7.26 (m, 1 H) 7.16-7.22 (m, 1 H) 6.68 (br. s., 1 H) 5.95 (br. s., 1 H) 5.76 (q, J = 8.75 Hz, 1 H) 4.97-5.11 (m, 2 H) 4.54-4.66 (m, 2 H) 4.47-4.54 (m, 1 H) 4.26-4.38 (m, 1H) 4.10-4.22 (m, 1 H) 2.89 (s, 6 H) 2.84- 2.88 (m, 2 H) 2.78-2.84 (m, 1 H) 2.69- 2.78 (m, 1 H) 2.50-2.63 (m, 1 H) 2.24 (q, J = 8.75 Hz, 1 H) 1.85-1.97 (m, 4 H) 1.77- 1.85 (m, 1 H) 1.69-1.77 (m, 2 H) 1.63- 1.68 (m, 1 H) 1.54 (d, J = 6.87 Hz, 6 H) 1.42- 1.51 (m, 4 H) 1.38-1.41 (m, 6 H) 1.36 (br. s., 9 H) 1.25-1.34 (m, 2 H) 1.10-1.25 (m, 1 H). LC-MS: purity 100% (UV), t_{R} 5.09 min m/z [M + H]⁺ 895.35 (MET/CR/1426). HRMS: Found: 895.4212, calculated for C₄₄H₆₂N₈O₈S₂ (M + H)⁺: 895.421. |
| 1776 | 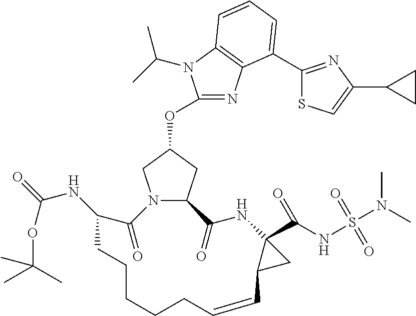 | 33 mg (17% yield) as a beige solid after preparative HPLC. ¹H NMR (500 MHz, CDCl₃) δ ppm 10.00 (br. s., 1 H) 8.13 (d, J = 7.63 Hz, 1 H) 7.28-7.31 (m, 1 H) 7.20 (t, J = 7.78 Hz, 1 H) 6.97 (s, 1 H) 6.67 (br. s., 1 H) 5.91-6.00 (m, 1 H) 5.76 (q, J = 9.16 Hz, 1 H) 4.97-5.08 (m, 2 H) 4.55-4.67 (m, 2 H) 4.46-4.54 (m, 1 H) 4.31 (ddd, J = 10.64, 7.67, 2.90 Hz, 1 H) 4.16 (dd, J = 11.37, 4.20 Hz, 1 H) 2.89 (s, 6 H) 2.73- 2.86 (m, 2 H) 2.53-2.62 (m, 1 H) 2.19- 2.26 (m, 1 H) 2.13-2.19 (m, 1H) 1.84- 1.96 (m, 2 H) 1.71-1.84 (m, 1 H) 1.54 (d, J = 6.71 Hz, 6 H) 1.40-1.51 (m, 5 H) 1.36 (s, 9 H) 1.25-1.33 (m, 2 H) 1.17 (br. s., 1 H) 0.94-1.02 (m, 4 H). LC-MS: purity 100% (UV), t_{R} 4.82 min m/z [M + H]⁺ 853.25 (MET/CR/1426). HRMS: Found: 853.3735, calculated for C₄₁H₅₆N₈O₈S₂ (M + H)⁺: 853.3741. |
| 1777 | 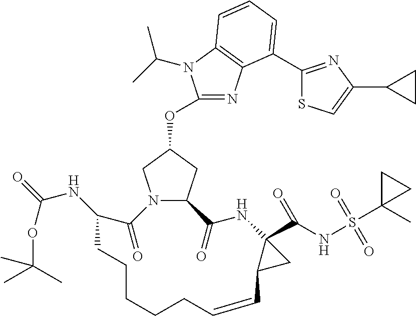 | 42 mg (21% yield) as an off-white solid after preparative HPLC. ¹H NMR (500 MHz, CDCl₃) δ ppm 10.12 (s, 1 H) 8.00-8.24 (m, 1 H) 7.25-7.32 (m, 2 H) 7.14-7.24 (m, 3 H) 7.09 (d, J = 7.78 Hz, 2 H) 6.75 (br. s., 1 H) 5.96 (br. s., 1 H) 5.73 (q, J = 8.70 Hz, 1 H) 4.97-5.11 (m, 2 H) 4.58-4.65 (m, 1H) 4.50-4.60 (m, 2 H) 4.28-4.37 (m, 1H) 4.07-4.27 (m, 3 H) 2.80-2.89 (m, 1H) 2.69-2.80 (m, 1 H) 2.51-2.64 (m, 1H) 2.45 (s, 3 H) 2.31 (s, 3 H) 2.23-2.35 (m, 1 H) 1.87-2.00 (m, 2 H) 1.74-1.87 (m, 2 H) 1.58-1.72 (m, 2 H) 1.54 (d, J = 6.87 Hz, 6 H) 1.50 (s, 3 H) 1.44-1.49 (m, 5 H) 1.36 (s, 9 H) 1.10-1.23 (m, 1 H) 0.76-0.89 (m, 2 H). LC-MS: purity 100% (UV), t_{R} 4.88 min m/z [M + H]⁺ 864.30 (MET/CR/1426). HRMS: Found: 864.3798, calculated for C₄₃H₅₇N₇O₈S₂ (M + H)⁺: 864.3788. |
| 1778 | 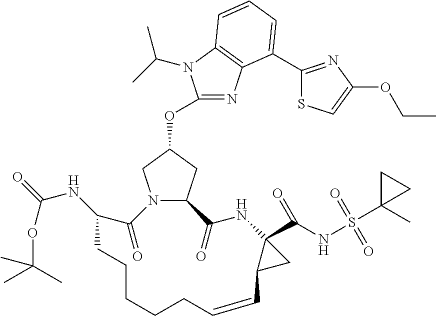 | 29 mg (27% yield) as an off-white solid after preparative HPLC. ¹H NMR (500 MHz, CDCl₃) δ ppm 10.00 (s, 1 H) 8.16 (d, J = 7.63 Hz, 1 H) 7.28-7.31 (m, 1 H) 7.21 (t, J = 7.93 Hz, 1 H) 6.62 (br. s., 1 H) 6.23 (s, 1 H) 5.94-6.00 (m, 1 H) 5.76 (q, J = 9.31 Hz, 1 H) 4.97-5.09 (m, 2 H) 4.56-4.67 (m, 2 H) 4.48-4.56 (m, 1 H) 4.28-4.36 (m, 1 H) 4.23 (q, J = 7.07 Hz, 2 H) 4.12- 4.19 (m, 1 H) 2.90 (s, 6 H) 2.79-2.86 (m, 2 H) 2.53-2.64 (m, 1 H) 2.17-2.28 (m, 1H) 1.86-1.97 (m, 2 H) 1.75-1.86 (m, 1H) 1.60-1.69 (m, 1 H) 1.53-1.58 (m, 8 H) 1.50 (t, J = 7.02 Hz, 3 H) 1.42-1.50 (m, 5 H) 1.37 (s, 9 H). LC-MS: purity 96% (UV), t_{R} 4.71 min m/z [M + H]⁺ 857.20 (MET/CR/1426. HRMS: Found: 857.3691, calculated for C₄₀H₅₆N₈O₉S₂ (M + H)⁺: 857.369. |

### 17.16 Synthesis of Compounds 1779-1780

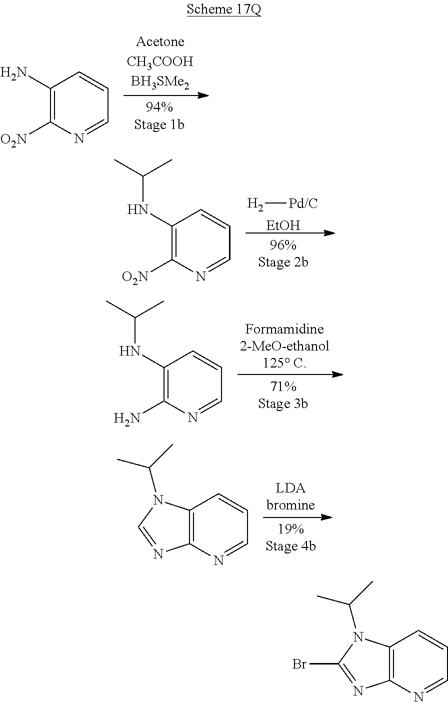

Stage 1b: 2-Nitro-3-amino-pyridine (4.67 g, 33.0 mmol, 1 eq.), acetone (4.8 mL, 66.0 mmol, 2.0 eq.) and dichloromethane (30 mL) were charged into a 100 mL round bottom flask and the reaction mixture cooled to 0° C. Borane-dimethyl sulphide complex (4.63 mL, 49.0 mmol, 1.5 eq.) was added dropwise. The reaction mixture was left to warm up to ambient temperature and stirring continued for 9 hours. The reaction was quenched by dropwise addition of concentrated aqueous ammonia (10 mL). The organic layer was washed with brine (25 mL), dried over magnesium sulfate, filtered and the solvent removed in vacuo to give 6.15 g (95 yield) of the title compound as a dark red oil which was used in the next step without further purification. ¹H NMR (500 MHz, CDCl₃) δ ppm 7.88 (dd, J=3.97, 1.37 Hz, 1H) 7.69 (br. s., 1H) 7.43 (dd, J=8.70, 3.97 Hz, 1H) 7.35 (d, J=8.54 Hz, 1H) 3.77-3.88 (m, 1H) 1.35 (d, J=6.26 Hz, 6H). LC-MS: purity 91% (UV), t_{R} 1.73 min m/z [M+H]⁺ 181.95 (MET/CR/1278).

Stage 2b: 2-Nitro-3-isopropylamino-pyridine (6.15 g, 32.0 mmol, 1 eq.) and ethanol (100 mL) were charged into a 250 mL round bottom flask fitted with a three way tap. 10% Palladium on charcoal (50% w/w water wet, 600 mg, 5 wt %) was added and the flask purged with nitrogen gas three times then with hydrogen gas three times. The reaction mixture was then stirred under an atmosphere of hydrogen for 15 hours. The catalyst was removed by filtration over micro-fibre paper and the solvent removed in vacuo to give 4.85 g (96% yield) of the title compound as a dark oil which was used in the next step without further purification. ¹H NMR (500 MHz, CDCl₃) δ ppm 7.59 (dd, J=5.03, 1.52 Hz, 1H) 6.81 (dd, J=7.77, 1.52 Hz, 1H) 6.70 (dd, J=7.77, 5.03 Hz, 1H) 4.20 (br. s., 2H) 3.56 (spt, J=6.22 Hz, 1H) 3.03 (br. s., 1H) 1.23 (d, J=6.24 Hz, 6H). LC-MS: purity 85% (UV), t_{R} 0.96 min m/z [M+H]⁺ 152.00 (MET/CR/1278).

Stage 3b: 2-Amino-3-isopropylamino-pyridine (4.85 g, 30.0 mmol, 1 eq.) and methoxyethanol (75 mL) were charged into a 250 mL round bottom flask fitted. Formamidine acetate (6.34 g, 60.0 mmol, 2 eq.) was added portion wise and the reaction mixture heated under reflux for 2 hours. The reaction mixture was allowed to cool to ambient temperature, the solvent was removed under reduced pressure and then the residue was diluted with ethyl acetate (100 mL). The organic phase was washed with water (100 mL) and saturated aqueous sodium hydrogen carbonate solution (100 mL). The combined aqueous washes were back extracted with ethyl acetate (100 mL) and dichloromethane (100 mL). All three organic phases were combined, dried over magnesium sulfate, filtered and the solvent removed in vacuo. The residue was purified by flash column chromatography using a methanol/dichloromethane gradient. After combining the relevant fractions, the solvent was removed in vacuo to give 3.73 g (70% yield) of the title compound as a brown oil which solidified on standing. ¹H NMR (500 MHz, CDCl₃) δ ppm 8.58 (dd, J=4.73, 1.53 Hz, 1H) 8.21 (s, 1H) 7.78 (dd, J=8.09, 1.53 Hz, 1H) 7.23 (dd, J=8.09, 4.73 Hz, 1H) 4.66 (spt, J=6.76 Hz, 1H) 1.65 (d, J=6.87 Hz, 6H). LC-MS: purity 93% (UV), t_{R} 0.72 min m/z [M+H]⁺ 161.90 (MET/CR/1278).

Stage 4b: 1-isopropyl-1H-imidazo[4,5-b]pyridine (400 mg, 2.48 mmol, 1 eq.) and tetrahydrofuran (8 mL) were charged into a 25 mL round bottom flask and the reaction mixture cooled down to −70° C. Lithium diisopropylamide solution (1.8 M, 2.07 mL, 3.72 mmol, 1.5 eq.) was added dropwise over 2 minutes. The reaction mixture was allowed to warm to 0° C. and stirring continued for 5 minutes. The reaction mixture was cooled to −70° C. and a solution of bromine (0.192 mL, 3.72 mmol, 1.5 eq.) in tetrahydrofuran (4 mL) was added in a rapid fashion. Stirring was continued for a further 15 hours while the reaction mixture warmed gently to ambient temperature. The reaction was quenched with acetic acid (50 mL) and the solvent removed in vacuo. The residue was purified by flash column chromatography using a methanol/dichloromethane gradient to give 115 mg (19% yield) of the title compound as a brown oil which crystallized out on standing. ¹H NMR (500 MHz, CDCl₃) δ ppm 8.56 (dd, J=4.88, 1.37 Hz, 1H) 7.93 (dd, J=8.16, 1.14 Hz, 1H) 7.25 (dd, J=8.24, 4.88 Hz, 1H) 5.00 (spt, J=7.02 Hz, 1H) 1.68 (d, J=7.02 Hz, 6H). LC-MS: purity 100% (UV), t_{R} 1.43 min m/z [M+H]⁺ 239.85/241.85 (MET/CR/1278).

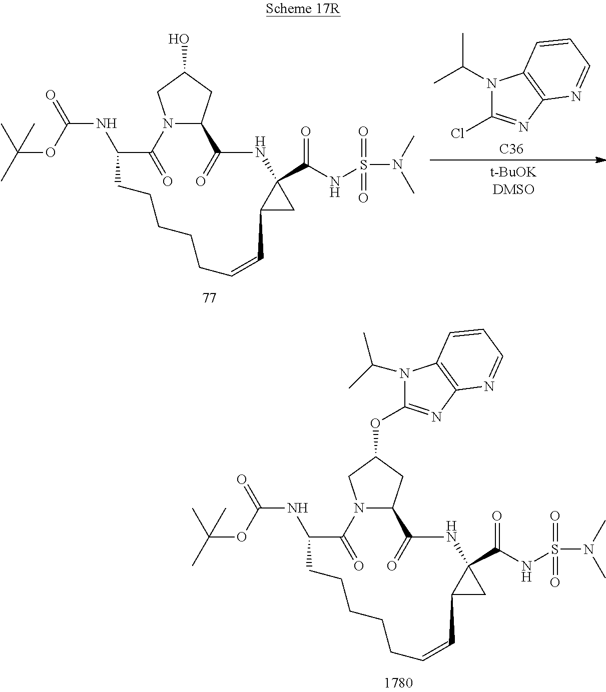

Compounds 1779 and 1780 was prepared using Scheme 17R.

TABLE 37
Compounds 1779-1780.

| Compound | Structure | Yield |
|---|---|---|
| 1779 | 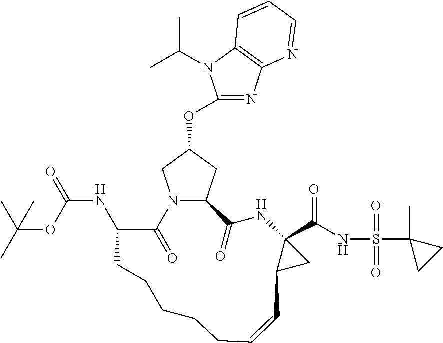 | 35 mg (20%) as a beige solid after preparative HPLC. ¹H NMR (500 MHz, CDCl₃) δ 9.95 (br. s., 1 H) 8.27 (d, J = 4.58 Hz, 1 H) 8.18 (br. s., 1 H) 7.51 (d, J = 7.78 Hz, 1 H) 7.03 (dd, J = 7.86, 5.11 Hz, 1 H) 5.93 (br. s., 1 H) 5.72 (dt, J = 9.46, 8.39 Hz, 1 H) 4.96-5.18 (m, 2 H) 4.61-4.73 (m, 1 H) 4.48-4.60 (m, 2 H) 4.24 - 4.37 (m, 1 H) 4.00-4.11 (m, 1 H) 2.77-2.90 (m, 1 H) 2.64-2.72 (m, 1 H) 2.63 (s, 3 H) 2.47-2.60 (m, 1 H) 2.33 (q, 1 H) 1.91-2.01 (m, 1 H) 1.78-1.90 (m, 2 H) 1.75 (d, J = 11.14 Hz, 1 H) 1.48-1.57 (m, 4 H) 1.46 (s, 3 H) 1.40-1.46 (m, 6 H) 1.35 (s, 9 H) 1.21-1.32 (m, 2 H) 0.76-0.87 (m, 2 H). LC-MS: purity 100% (UV), t_{R} 4.04 min m/z [M + H]⁺ 742.80 (MET/CR/1416). HRMS: Found: 742.3596, calculated for C₃₆H₅₁N₇O₈S (M + H)⁺: 742.3598. |
| 1780 | 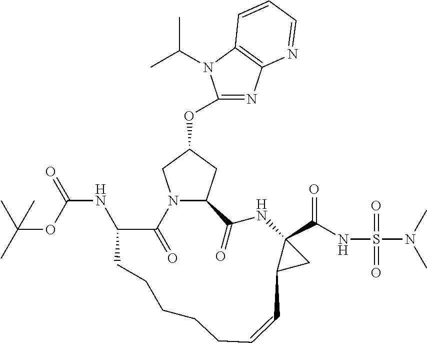 | 47 mg (26%) as a beige solid after preparative HPLC. ¹H NMR (500 MHz, CDCl₃) δ ppm 9.80 (br. s., 1 H) 8.31-8.54 (m, 1 H) 8.22-8.31 (m, 1 H) 7.51 (d, J = 7.63 Hz, 1 H) 7.03 (dd, J = 7.86, 5.11 Hz, 1 H) 5.92 (br. s., 1 H) 5.68-5.79 (m, 1 H) 5.11 (t, J = 9.31 Hz, 1 H) 5.00-5.08 (m, 1 H) 4.61-4.72 (m, 1 H) 4.46- 4.59 (m, 2 H) 4.26-4.35 (m, 1H) 3.98-4.09 (m, 1 H) 2.87 (s, 6 H) 2.77-2.85 (m, 1 H) 2.64-2.70 (m, 1 H) 2.63 (s, 3 H) 2.50-2.60 (m, 1 H) 2.25-2.38 (m, 1 H) 1.89-2.01 (m, 1 H) 1.76-1.88 (m, 2 H) 1.47- 1.57 (m, 4 H) 1.38-1.47 (m, 5 H) 1.35 (s, 9 H) 1.23-1.32 (m, 2 H). LC-MS: purity 100% (UV), t_{R} 3.95 min m/z [M + H]⁺ 731.35 (MET/CR/1416). HRMS: Found: 742.3596, calculated for C₃₆H₅₁N₇O₈S (M + H)⁺: 742.3598. |

### Example 18

### Examples of NS3-NS4 activity

NS3-NS4 inhibition activity can be determined using known assay methods. For example, NS3/NS4 complexes may be formed and inhibitory concentrations of test compounds determined as described in U.S. Patent Application Publication Number 2007/0054842 paragraph numbers 1497-1509, which is incorporated herein by reference in its entirety. Similarly, hepatitis C replicon EC₅₀ may be determined using known assay methods such as described in U.S. Patent Application Publication Number 2007/0054842 paragraph numbers 1510-1515, which is incorporated herein by reference in its entirety. Assays may be conducted at ambient temperature (23° C.) in assay buffer containing 50 mM Tris-HCl, pH 7.5, 15% glycerol, 0.6 mM Lauryldimethylamine Oxide (LDAO), 25 μM NS4A peptide, and 10 mM Dithiothreitol (DTT).

Inhibition of NS3/NS4 activity was determined for several compounds exemplified herein and is presented in Table 14.

TABLE 38
Examples NS3-NS4 activity.

| Compound | EC₅₀ (nM) | IC₅₀ (nM) |
|---|---|---|
| 101 | B | C |
| 102 | C | C |
| 103 | C | C |
| 104 | C | C |
| 105 | C | C |
| 106 | C | C |
| 107 | C | C |
| 108 | C | C |
| 109 | C | C |
| 110 | C | C |
| 111 | C | C |
| 112 | C | C |
| 113 | C | C |
| 114 | C | C |
| 115 | C | C |
| 116 | B | C |
| 117 | C | C |
| 118 | B | C |
| 119 | C | C |
| 120 | C | C |
| 121 | B | C |
| 122 | B | C |
| 123 | B | C |
| 124 | B | C |
| 125 | C | C |
| 126 | C | C |
| 127 | C | C |
| 128 | C | C |
| 129 | B | C |
| 200 | C | C |
| 201 | B | C |
| 202 | A | C |
| 203 | B | C |
| 204 | B | C |
| 205 | C | C |
| 206 | C | C |
| 207 | C | C |
| 208 | A | C |
| 209 | B | C |
| 210 | C | C |
| 211 | C | C |
| 212 | C | C |
| 213 | C | C |
| 214 | C | C |
| 215 | C | C |
| 216 | C | C |
| 217 | C | C |
| 218 | C | C |
| 219 | C | C |
| 220 | C | C |
| 221 | C | C |
| 222 | C | C |
| 223 | C | C |
| 224 | C | C |
| 225 | C | C |
| 226 | C | C |
| 227 | C | C |
| 228 | C | C |
| 229 | C | C |
| 230 | C | C |
| 231 | B | C |
| 232 | B | C |
| 233 | B | C |
| 234 | C | C |
| 235 | C | C |
| 236 | C | C |
| 237 | C | C |
| 238 | B | C |
| 239 | B | C |
| 240 | B | C |
| 241 | B | C |
| 242 | B | C |
| 243 | A | C |
| 244 | B | C |
| 245 | A | C |
| 246 | A | C |
| 247 | A | C |
| 248 | B | C |
| 249 | A | C |
| 250 | A | C |
| 251 | A | C |
| 252 | B | C |
| 253 | A | C |
| 254 | B | C |
| 255 | B | C |
| 256 | C | C |
| 257 | B | C |
| 258 | C | C |
| 259 | B | C |
| 260 | B | C |
| 261 | B | C |
| 262 | B | C |
| 263 | B | C |
| 264 | C | C |
| 265 | B | C |
| 266 | C | C |
| 267 | A | C |
| 268 | B | C |
| 269 | B | C |
| 270 | A | C |
| 271 | B | C |
| 272 | B | C |
| 273 | B | C |
| 274 | B | C |
| 275 | B | C |
| 276 | A | C |
| 277 | C | C |
| 278 | B | C |
| 279 | A | C |
| 280 | C | C |
| 281 | C | C |
| 282 | B | C |
| 283 | C | C |
| 284 | C | C |
| 285 | C | C |
| 286 | C | C |
| 287 | B | C |
| 288 | C | C |
| 289 | B | C |
| 290 | B | C |
| 291 | C | C |
| 292 | C | C |
| 293 | B | C |
| 294 | A | C |
| 295 | A | C |
| 296 | A | C |
| 297 | A | C |
| 298 | A | C |
| 299 | B | C |
| 301 | B | C |
| 302 | C | C |
| 303 | C | C |
| 304 | B | C |
| 305 | C | C |
| 306 | B | C |
| 307 | B | C |
| 308 | B | C |
| 309 | B | C |
| 310 | B | C |
| 311 | B | C |
| 312 | B | C |
| 401 | B | C |
| 501 | A | C |
| 502 | B | C |
| 503 | B | C |
| 504 | B | C |
| 505 | A | C |
| 506 | A | C |
| 601 | C | C |
| 602 | B | C |
| 701 | A | C |
| 702 | B | C |
| 801 | B | C |
| 802 | A | C |
| 803 | B | C |
| 804 | B | C |
| 805 | A | C |
| 901 | C | C |
| 1001 | C | C |
| 1002 | C | C |
| 1003 | B | A |
| 1004 | B | C |
| 1005 | A | C |
| 1005S | A | C |
| 1101 | C | C |
| 1101S | C | C |
| 1201 | A | C |
| 1202 | B | C |
| 1203 | B | C |
| 1204 | C | C |
| 1205 | C | C |
| 1206 | C | C |
| 1207 | B | C |
| 1208 | B | C |
| 1209 | C | C |
| 1210 | C | C |
| 1211 | C | C |
| 1212 | C | C |
| 1213 | B | C |
| 1214 | C | C |
| 1215 | C | C |
| 1216 | C | C |
| 1217 | C | C |
| 1218 | A | C |
| 1219 | C | C |
| 1220 | C | C |
| 1221 | C | C |
| 1222 | C | C |
| 1223 | C | C |
| 1224 | C | C |
| 1251 | B | C |
| 1252 | B | C |
| 1253 | A | C |
| 1401 | C | C |
| 1402 | C | C |
| 1403 | C | C |
| 1404 | C | C |
| 1405 | B | C |
| 1406 | A | C |
| 1407 | B | C |
| 1408 | B | C |
| 1409 | C | C |
| 1410 | B | C |
| 1411 | B | NA |
| 1412 | C | C |
| 1413 | C | NA |
| 1414 | C | C |
| 1415 | B | C |
| 1416 | B | NA |
| 1417 | B | NA |
| 1418 | C | C |
| 1419 | C | C |
| 1420 | C | C |
| 1421 | C | C |
| 1422 | C | C |
| 1423 | C | C |
| 1424 | C | C |
| 1425 | B | C |
| 1426 | B | C |
| 1427 | C | C |
| 1428 | C | C |
| 1429 | C | C |
| 1430 | B | C |
| 1431 | B | C |
| 1432 | B | C |
| 1433 | C | C |
| 1434 | C | C |
| 1435 | C | C |
| 1436 | B | C |
| 1501 | A | B |
| 1502 | A | C |
| 1503 | A | C |
| 1504 | A | B |
| 1505 | A | C |
| 1506 | B | C |
| 1601 | A | C |
| 1701 | B | C |
| 1702 | C | C |
| 1703 | B | C |
| 1704 | C | C |
| 1705 | C | C |
| 1706 | C | C |
| 1707 | C | C |
| 1708 | C | C |
| 1709 | B | C |
| 1710 | B | C |
| 1711 | C | C |
| 1712 | C | C |
| 1713 | B | C |
| 1714 | B | C |
| 1715 | B | C |
| 1716 | B | C |
| 1717 | B | C |
| 1718 | C | C |
| 1719 | C | C |
| 1720 | C | C |
| 1721 | C | C |
| 1722 | B | C |
| 1723 | C | C |
| 1724 | B | C |
| 1725 | C | C |
| 1726 | C | C |
| 1727 | C | C |
| 1728 | A | C |
| 1729 | B | C |
| 1730 | C | C |
| 1731 | C | C |
| 1732 | C | C |
| 1733 | C | C |
| 1734 | A | C |
| 1735 | C | C |
| 1736 | C | C |
| 1737 | C | C |
| 1738 | C | NA |
| 1739 | B | C |
| 1740 | B | C |
| 1741 | C | C |
| 1742 | B | C |
| 1743 | B | C |
| 1744 | C | C |
| 1745 | B | C |
| 1746 | B | C |
| 1747 | B | C |
| 1748 | B | C |
| 1749 | B | C |
| 1750 | B | C |
| 1751 | B | C |
| 1752 | B | C |
| 1753 | B | C |
| 1754 | B | C |
| 1755 | B | C |
| 1756 | B | C |
| 1757 | C | C |
| 1758 | B | C |
| 1759 | C | C |
| 1760 | B | C |
| 1761 | B | C |
| 1762 | A | B |
| 1763 | A | B |
| 1764 | C | C |
| 1765 | C | C |
| 1766 | C | C |
| 1767 | C | C |
| 1768 | C | C |
| 1769 | C | C |
| 1770 | B | C |
| 1771 | C | C |
| 1772 | B | C |
| 1773 | C | C |
| 1774 | B | C |
| 1775 | B | C |
| 1776 | C | C |
| 1777 | C | C |
| 1778 | C | C |
| 1779 | B | C |
| 1780 | B | C |

A indicates an EC₅₀ or IC₅₀ > 100 nM
B indicates an EC₅₀ or IC₅₀ between 10 and 100 nM
C indicates an EC₅₀ or IC₅₀ of less than 10 nM
NA means the data is not available

What is claimed is:

1. A compound having the structure of Formula I or XII: 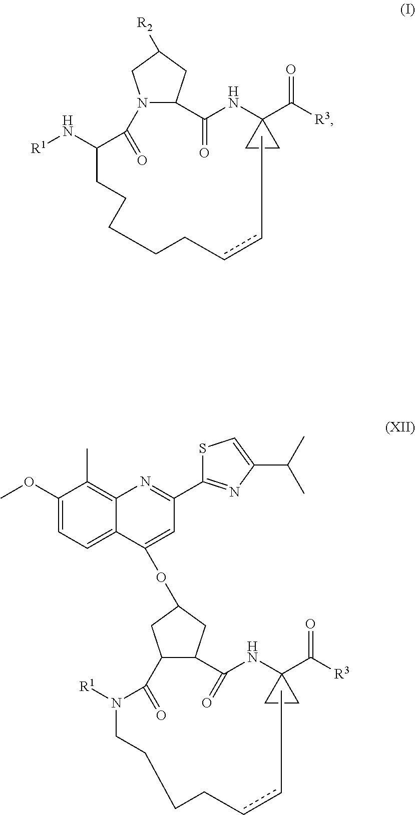
or a pharmaceutically acceptable salt or prodrug thereof wherein:
(a) R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, NHC(O)NR^{1a}R^{1b}, —C(O)OR^{1c}, and heteroaryl;
R^{1e} is selected from the group consisting of t-butyl, cycloalkyl, and heterocyclyl;
R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl;
R^{1c} and R^{1d} are each separately selected from the group consisting of —H (hydrogen), C₁₋₄ alkoxy, C₁₋₆ alkyl, C₃₋₇ cycloalkyl, aryl, arylalkyl and heteroaryl;
(b) R² is selected from the group consisting of 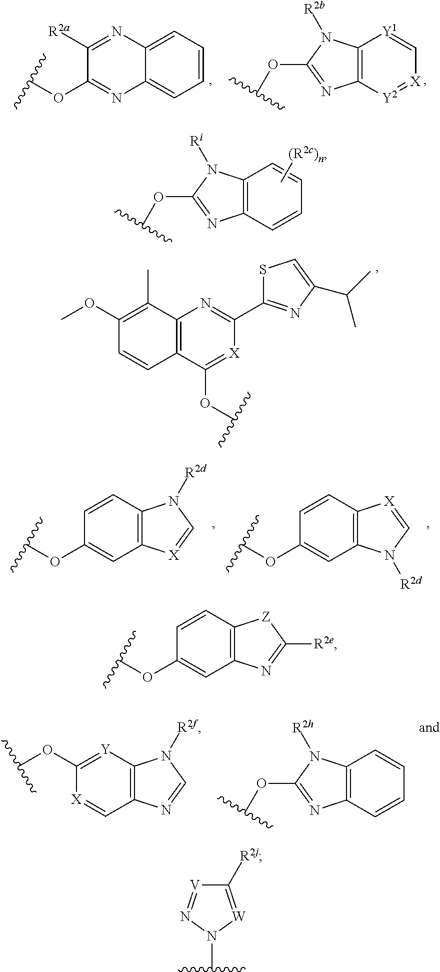
X, Y, Y¹, and Y² are each independently selected from —CH— or —N—,
wherein X and Y are not both —CH—, and X, Y¹, and Y² are not all —CH—,
Z is O (oxygen) or S (sulfur);
V and W are each independently selected from —CR^{2k}— or —N—,
wherein V and W are not both —CR^{2k}—;
n is 1, 2 or 3;
R^{2j} and R^{2k} are each independently selected from the group consisting of H, halo, optionally substituted aryl, optionally substituted heteroaryl; or R^{2j} and R^{2k} together form an aryl ring optionally substituted by 1-3 R^{2g};
R^{2a}, R^{2e} and R² are each independently selected from the group consisting of halo, —C(O)OR^{I}', —C(O)NR′R″, —NR′R″, —NHC(O)NR′R″, —NHC(O)OR^{1c}, —NHS(O)₂R^{1c}, C₁₋₆ alkyl optionally substituted with up to 5 fluoro, C₂₋₆ alkenyl, C₃₋₇ cycloalkyl, optionally substituted C₁₋₆ alkoxy, optionally substituted aryl and optionally substituted heteroaryl;
each R^{2c} is independently selected from the group consisting of halo, —C(O)OR^{1c}, —C(O)NR′R″, —NR′R″, —NHC(O)NR′R″, —NHC(O)OR^{1c}, —NHS(O)₂R^{1c}, C₁₋₆ alkyl, C₂₋₆ alkenyl, C₃₋₇ cycloalkyl, C₁₋₆ alkoxy, arylalkyl, polycyclic moiety, aryl, and heteroaryl, said C₁₋₆ alkyl, C₂₋₆ alkenyl, C₃₋₇ cycloalkyl, C₁₋₆ alkoxy, arylalkyl, polycyclic moiety, aryl, and heteroaryl each optionally substituted with one or more R¹²;
each R¹² is independently selected from the group consisting of C₁₋₆ alkyl, C₃₋₇ cycloalkyl, C₁₋₆ alkoxy, heteroaryl, arylalkyl, aryl, —F (fluoro), —Cl (Chloro), —CN, —CF₃, —OCF₃, —C(O)NR′R″ and —NR′R″, wherein said C₁₋₆ alkyl, C₃₋₇ cycloalkyl, C₁₋₆ alkoxy, heteroaryl, arylalkyl, and aryl are each optionally substituted with one or more R^{12a};
each R^{12a} is independently selected from the group consisting of —F, —Cl, —CF₃, —OCF₃, C₁₋₆ alkyl, C₁₋₆ alkoxy, and aryl;
each NR′R″ is separately selected wherein R′ and R″ are each independently selected from the group consisting of —H (hydrogen), halo, —C(O)NR′R″, optionally substituted C₁₋₆ alkyl, optionally substituted C₂₋₆ alkenyl, optionally substituted C₁₋₆ alkoxy, optionally substituted aryl, optionally substituted arylalkyl and optionally substituted heteroaryl; or R′ and R″ are taken together with the nitrogen to which they are attached to form heterocyclyl;
R^{2b}, R^{2d} and R^{2f} are each independently selected from the group consisting of C₁₋₆ alkyl optionally substituted with up to 5 fluoro, C₂₋₆ alkenyl, C₃₋₇ cycloalkyl, arylalkyl, optionally substituted aryl and optionally substituted heteroaryl;
R^{2b} is selected from the group consisting of propyl, butyl and phenyl;
Rⁱ is C₁₋₆ alkyl optionally substituted with up to 5 fluoro;
(c) R³ is —OH, —NHS(O)₂R^{3a}, —NHS(O)₂OR^{3a} or —NHS(O)₂NR^{3b}R^{3c};
where R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro;
R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇ cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro;
or R^{3b} and R^{3c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl;
each t is independently 0, 1 or 2;
each q is independently 0, 1 or 2;
(d) any bond represented by a dashed and solid line represents a bond selected from the group consisting of a single bond and a double bond;
(e) provided that if R² is 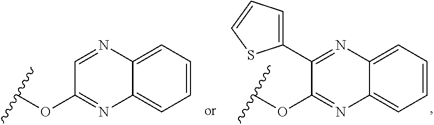
then R¹ is not phenyl;
(f) provided that if R² is 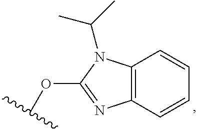
then R¹ is not —C(O)O-t-butyl, phenyl or phenyl substituted with one or more substituents selected from the group consisting of fluoro, chloro and —CF₃;
(g) provided that if R² is 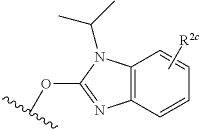
and R^{2c} is —F or methyl, then R¹ is not —C(O)O-t-butyl or phenyl;
(h) provided that if R² is 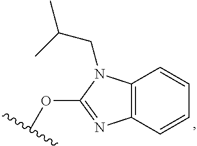
then R¹ is not —C(O)O-t-butyl or phenyl substituted with one or more substituents selected from the group consisting of fluoro and —CF₃; and
(i) provided that if R² is 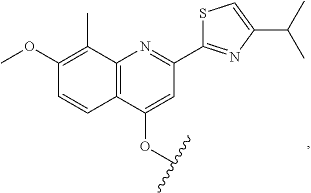
then R¹ is not —C(O)O-t-butyl, benzoxazyl, t-butylthiazyl, phenyl or phenyl substituted with one or more substituents selected from the group consisting of fluoro, chloro, methyl, —CF₃ and —OCF₃.

2. The compound of claim 1 having the structure:

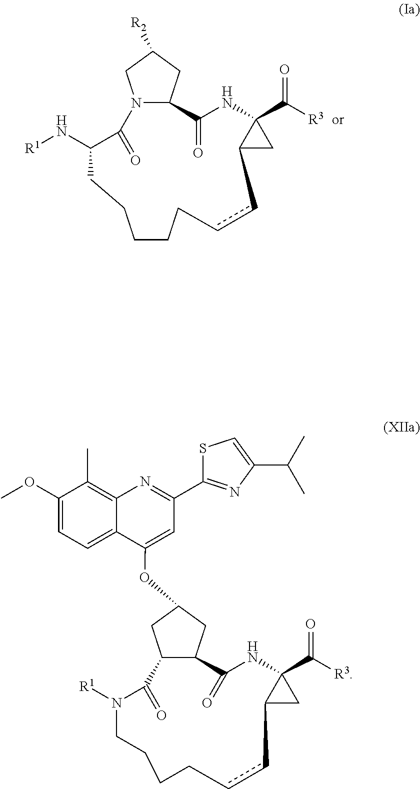

3. The compound of claim 1, wherein R¹ is selected from the group consisting of —C(O)O—R^{1e}, optionally substituted heteroaryl, and aryl optionally substituted with one or more substitutents each independently selected from the group consisting of C₁₋₆ alkyl, fluoro, amino, —CF₃, —OCF₃, —C(O)NR^{1a}R^{1b}, —NHC(O)NR^{1a}R^{1b}, —C(O)OH, and oxazolyl.

4. The compound of claim 3, wherein R^{1a} and R^{1b} are taken together with the nitrogen to which they are attached to form piperazinyl or morpholinyl bonded to the parent structure through a nitrogen, each optionally substituted with one or more substituents independently selected from C₁₋₆ alkyl, C₂₋₆ alkenyl, C₂₋₆ alkynyl, —C(O)OR^{1c}, —C(O)R^{1d}, hydroxy-C₁₋₆alkyl, amino-C₁₋₆alkyl, aryl-C₁₋₆alkyl, optionally substituted aryl, and heteroaryl; and R^{1c} and R^{1d} are each separately selected from the group consisting of —H (hydrogen), C₁₋₄ alkoxy, C₁₋₆ alkyl, C₃₋₇ cycloalkyl, aryl, arylalkyl and heteroaryl.

5. The compound of claim 1, wherein R¹ is aryl optionally substituted with one or more substitutents each independently selected from the group consisting of —C(O)NR^{1a}R^{1b} and —NHC(O)NR^{1a}R^{1b}, wherein R^{1a} and R^{1b} are taken together with the nitrogen to which they are attached to form piperazinyl or morpholinyl, each optionally substituted with C₁₋₆ alkyl, hydroxy-C₁₋₆ alkyl, amino-C₁₋₆ alkyl, aryl-C₁₋₆ alkyl, —C(O)OR^{1c}, —C(O)R^{1d}, optionally substituted aryl, and heteroaryl.

6. The compound of claim 5 wherein R^{1a} and R^{1b} are taken together with the nitrogen to which they are attached to form: 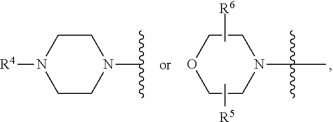
wherein
R⁴ is selected from the group consisting of —H, C₁₋₆ alkyl optionally substituted with one or more amine, aryl or hydroxy, aryl optionally substituted with C₁₋₄ alkyl, —CF₃, or —OCF₃, and —C(O)R^{4a}, where R^{4a} is selected from the group consisting of C₁₋₄ alkoxy, C₃₋₇ cycloalkyl and aryl; and
R⁵ and R⁶ are each independently —H or C₁₋₆ alkyl optionally substituted with phenyl.

7. The compound of claim 1, wherein:
R² is selected from the group consisting of 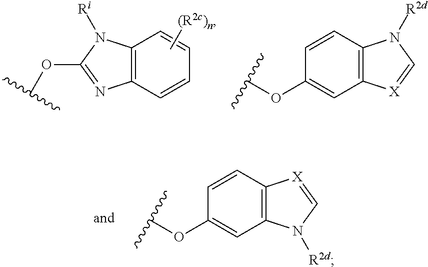
each R^{2c} is independently selected from the group consisting of —CF₃, —Br, —Cl, —C(O)OH, —C(O)NR′R″, —NR′R″, —NHC(O)NR′R″, —NHC(O)OR^{1c}, —NHS(O)₂R^{1c}, C₁₋₆ alkyl, C₂₋₆ alkenyl, C₁₋₆ alkoxy, polycyclic moiety, phenyl, and heteroaryl, said C₁₋₆ alkyl, C₂₋₆ alkenyl, C₁₋₆ alkoxy, polycyclic moiety, aryl, and heteroaryl each optionally substituted with one or more R¹²;
each R¹² is independently selected from the group consisting of C₁₋₆ alkyl, C₃₋₇ cycloalkyl, C₁₋₆ alkoxy, pyridinyl, phenylalkyl, phenyl, —F (fluoro), —Cl (Chloro), —CN, —CF₃, —OCF₃, —C(O)NR′R″, morpholinyl, pyrrolidinyl, piperidiny, C₃₋₇ cycloalkyl-alkyl, wherein said C₁₋₆ alkyl, C₃₋₇ cycloalkyl, C₁₋₆ alkoxy, pyridinyl, phenylalkyl, phenyl, morpholinyl, pyrrolidinyl, piperidiny, are each optionally substituted with one or more R^{12a};
each NR′R″ is separately selected wherein R′ and R″ are each independently selected from the group consisting of —H (hydrogen), —F, —Cl, —C(O)NR′R″, C₁₋₆ alkyl, C₂₋₆ alkenyl, C₁₋₆ alkoxy, phenyl, phenylalkyl, and heteroaryl;
each R^{12a} is independently selected from the group consisting of —F, —C₁, C₁₋₆ alkyl, C₁₋₆ alkoxy, C₃₋₇ cycloalkyl, and aryl;
R^{2d} is selected from the group consisting of C₁₋₆ alkyl optionally substituted with up to 5 fluoro, C₃₋₇ cycloalkyl, arylalkyl, optionally substituted aryl and optionally substituted heteroaryl; and
Rⁱ is ethyl or i-propyl.

8. The compound of claim 1, wherein
R² is 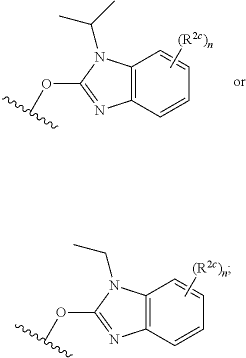
each R^{2c} is independently selected from the group consisting of —CF₃, —Br, —Cl, —C(O)OH, —C(O)NR′R″, —NR′R″, —NHC(O)NR′R″, —NHC(O)OR^{1c}, —NHS(O)₂R^{1c}, C₁₋₆ alkyl, C₂₋₆ alkenyl, C₁₋₆ alkoxy, polycyclic moiety, phenyl, and heteroaryl, said C₁₋₆ alkyl, C₂₋₆ alkenyl, C₁₋₆ alkoxy, polycyclic moiety, aryl, and heteroaryl each optionally substituted with one or more R¹²;
each R¹² is independently selected from the group consisting of C₁₋₆ alkyl, C₃₋₇ cycloalkyl, C₁₋₆ alkoxy, pyridinyl, phenylalkyl, phenyl, —F (fluoro), —Cl (Chloro), —CN, —CF₃, —OCF₃, —C(O)NR′R″, morpholinyl, pyrrolidinyl, piperidiny, C₃₋₇ cycloalkyl-alkyl, wherein said C₁₋₆ alkyl, C₃₋₇ cycloalkyl, C₁₋₆ alkoxy, pyridinyl, phenylalkyl, phenyl, morpholinyl, pyrrolidinyl, piperidiny, are each optionally substituted with one or more R^{12a};
each NR′R″ is separately selected wherein R′ and R″ are each independently selected from the group consisting of —H (hydrogen), —F, —Cl, —C(O)NR′R″, C₁₋₆ alkyl, C₂₋₆ alkenyl, C₁₋₆ alkoxy, phenyl, phenylalkyl, and heteroaryl;
each R^{12a} is independently selected from the group consisting of —F, —C₁, C₁₋₆ alkyl, C₁₋₆ alkoxy, C₃₋₇ cycloalkyl, and aryl.

9. The compound of claim 8, wherein R¹ is aryl, —C(O)OR^{1e}, or . . . optionally substituted heteroaryl; and R³ is —NHS(O)₂R^{3a} or —NHS(O)₂NR^{3b}R^{3c}; where R^{3a} is selected from the group consisting of C₁₋₆ alkyl and —(CH₂)_{q}C₃₋₇cycloalkyl, each optionally substituted with C₁₋₆ alkyl.

10. The compound of claim 1, wherein
R¹ is aryl substituted with one or more substitutents each independently selected from the group consisting of halo, amino, C₁₋₆ alkoxy optionally substituted with up to 5 fluoro, —COOH, —C(O)NR^{1a}R^{1b}, —NHC(O)NR^{1a}R^{1b} and heteroaryl;
R² is 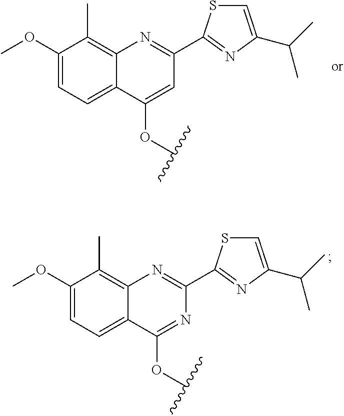
and
R³ is —OH, —NHS(O)₂R^{3a}, —NHS(O)₂OR^{3a} or —NHS(O)₂NR^{3b}R^{3c}; where R^{1a} is selected from the group consisting of C₁₋₆ alkyl and —(CH₂)_{q}C₃₋₇cycloalkyl, each optionally substituted with C₁₋₆ alkyl.

11. The compound of claim 10, wherein:
R¹ is aryl substituted with one or more substitutents each independently selected from the group consisting of —C(O)NR^{1a}R^{1b} and —NHC(O)NR^{1a}R^{1b};
R^{1a} and R^{1b} are taken together with the nitrogen to which they are attached to form piperazinyl or morpholinyl, each optionally substituted with one or more substituents independently selected from C₁₋₆ alkyl, C₂₋₆ alkenyl, C₂₋₆ alkynyl, —C(O)OR^{1c}, —C(O)R^{1d}, hydroxy-C₁₋₆alkyl, amino-C₁₋₆alkyl, aryl-C₁₋₆alkyl, aryl optionally substituted with C₁₋₆ alkyl or C₁₋₆ alkyl substituted with up to 5 fluoro, and heteroaryl; and
R^{1c} and R^{1d} are each separately selected from the group consisting of —H, C₁₋₄ alkoxy, C₁₋₆ alkyl, C₃₋₇ cycloalkyl, aryl, arylalkyl and heteroaryl.

12. The compound of claim 10, wherein R¹ is phenyl substituted with one or more substitutents each independently selected from the group consisting of —C(O)NR^{1a}R^{1b}, NHC(O)NR^{1a}R^{1b} and heteroaryl; and R³ is —NHS(O)₂R^{3a} or —NHS(O)₂NR^{3b}R^{3c}, where R^{1a} is C₃₋₇cycloalkyl optionally substituted with methyl, and R^{3b} and R^{3c} are methyl.

13. The compound of claim 10, wherein the compound is selected from the group consisting of Compounds 101-129, 601-602, 901, 1001-1002, and 1733.

14. A compound having the structure of Formula IIa-1: 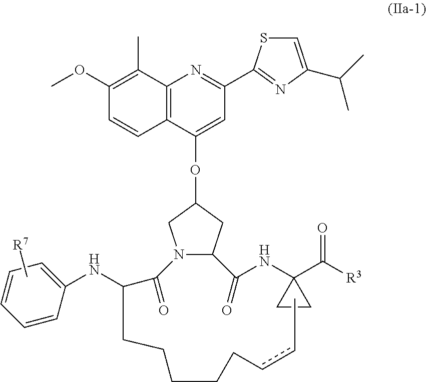
or a pharmaceutically acceptable salt or prodrug thereof wherein:
(a) R³ is —OH, —NHS(O)₂R^{3a}, —NHS(O)₂OR^{3a} or —NHS(O)₂NR^{3b}R^{3c};
where R^{1a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro;
R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro;
or R^{3b} and R^{3c} are taken together with the nitrogen to which they are attached to form a three- to six-membered heterocyclic ring, bonded to the parent structure through a nitrogen, and the heterocylic ring is optionally substituted with one or more substituents each independently selected from the group consisting of halo, cyano, nitro, C₁₋₆ alkyl, C₁₋₆ alkoxy, and phenyl;
each t is independently 0, 1 or 2;
each q is independently 0, 1 or 2;
(b) R⁷ is selected from the group consisting of —NH₂, —NH₂.HCl, —COOH, —C(O)NR^{1a}R^{1b}, NHC(O)NR^{1a}R^{1b} and heteroaryl containing 1-3 heteroatoms independently selected from N or O;
R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl;
R^{1c} and R^{1d} are each separately selected from the group consisting of H (hydrogen), C₁₋₄ alkoxy, C₁₋₆ alkyl, C₃₋₇ cycloalkyl, aryl, arylalkyl and heteroaryl; and
(c) any bond represented by a dashed and solid line represents a bond selected from the group consisting of a single bond and a double bond.

15. The compound of claim 14, wherein:
R³ is —OH, —NHS(O)₂R^{3a}, —NHS(O)₂OR^{3a} or —NHS(O)₂NR^{3b}R^{3c}, where R^{1a} is C₃₋₇cycloalkyl optionally substituted with methyl, and R^{3b} and R^{3c} are methyl; and
R⁷ is selected from the group consisting of —NH₂, —NH₂.HCl, —COOH, —C(O)NR^{1a}R^{1b}, —NHC(O)NR^{1a}R^{1b} and heteroaryl containing 1-3 heteroatoms independently selected from N or O, wherein R^{1a} and R^{1b} are taken together with the nitrogen to which they are attached to form piperazinyl or morpholinyl, each optionally substituted with one or more substituents independently selected from C₁₋₆ alkyl, —C(O)OR^{1c}, —C(O)R^{d}, hydroxy-C₁₋₆ alkyl, amino-C₁₋₆ alkyl, aryl-C₁₋₆ alkyl, phenyl optionally substituted with C₁₋₆ alkyl or —CF₃, and heteroaryl.

16. A compound having the structure of Formula III or IV 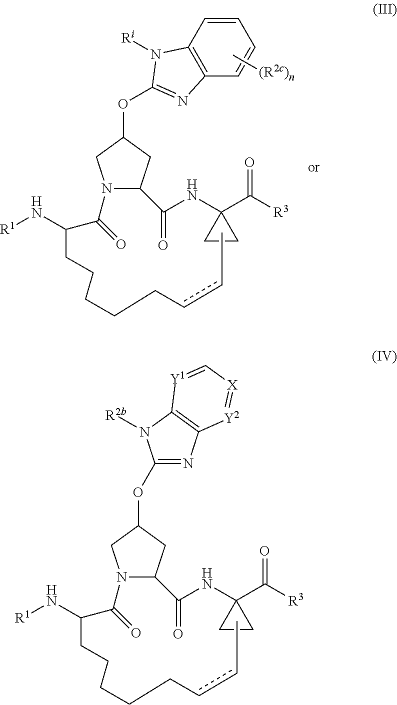
or a pharmaceutically acceptable salt or prodrug thereof wherein:
(a) R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NHC(O)NR^{1a}R^{1b}, —C(O)OR^{1c}, and heteroaryl;
R^{1e} is selected from the group consisting of t-butyl, cycloalkyl, and heterocyclyl;
R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)R^{1c}, —C(O)R^{d}, optionally substituted aryl, and optionally substituted heteroaryl;
R^{1c} and R^{1d} are each separately selected from the group consisting of —H, C₁₋₄ alkoxy, C₁₋₆ alkyl, C₃₋₇ cycloalkyl, aryl, arylalkyl and heteroaryl;
(b) X, Y, Y¹, and Y² are each independently selected from —CH— or —N—, wherein X and Y are not both —CH—, and X, Y¹, and Y² are not all —CH—;
(c) R^{2b} is selected from the group consisting of C₁₋₆ alkyl optionally substituted with up to 5 fluoro, C₂₋₆ alkenyl, C₃₋₇ cycloalkyl, arylalkyl, optionally substituted aryl and optionally substituted heteroaryl;
(d) each R^{2c} is independently selected from the group consisting of halo, —C(O)OR^{1c}, —C(O)NR′R″, —NR′R″, —NHC(O)NR′R″, —NHC(O)OR^{1c}, —NHS(O)₂R^{1c}, C₂₋₆ alkyl, C₂₋₆ alkenyl, C₃₋₇ cycloalkyl, C₁₋₆ alkoxy, arylalkyl, polycyclic moiety, aryl, and heteroaryl, said C₂₋₆ alkyl, C₂₋₆ alkenyl, C₃₋₇ cycloalkyl, alkoxy, arylalkyl, polycyclic moiety, aryl, and heteroaryl each optionally substituted with one or more R¹²;
each R¹² is independently selected from the group consisting of C₁₋₆ alkyl, C₃₋₇ cycloalkyl, alkoxy, heteroaryl, arylalkyl, aryl, —F (fluoro), —Cl (Chloro), —CN, —CF₃, —C(O)NR′R″ and —NR′R″, wherein said C₁₋₆ alkyl, C₃₋₇ cycloalkyl, alkoxy, heteroaryl, arylalkyl, cycloalkylalkyl, and aryl are each optionally substituted with one or more R^{12a};
each R^{12a} is independently selected from the group consisting of —F, —Cl, —CF₃, —OCF₃, C₁₋₆ alkyl, C₁₋₆ alkoxy, C₃₋₇ cycloalkyl, and aryl;
each NR′R″ is separately selected wherein R′ and R″ are each independently selected from the group consisting of —H (hydrogen), halo, —C(O)NR′R″, optionally substituted C₁₋₆ alkyl, optionally substituted C₂₋₆ alkenyl, optionally substituted C₁₋₆ alkoxy, optionally substituted aryl, optionally substituted arylalkyl and optionally substituted heteroaryl; or R′ and R″ are taken together with the nitrogen to which they are attached to form heterocyclyl;
(e) Rⁱ is C₁₋₆ alkyl optionally substituted with up to 5 fluoro;
(f) R³ is —OH, —NHS(O)₂R^{3a}, —NHS(O)₂OR^{3a} or —NHS(O)₂NR^{3b}R^{3c}; where R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro;
wherein R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro;
or R^{3b} and R^{3c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl;
each t is independently 0, 1 or 2;
each q is independently 0, 1 or 2;
(g) n is 1, 2 or 3; and
(h) any bond represented by a dashed and solid line represents a bond selected from the group consisting of a single bond and a double bond.

17. The compound of claim 16 having the structure of the following formulas:

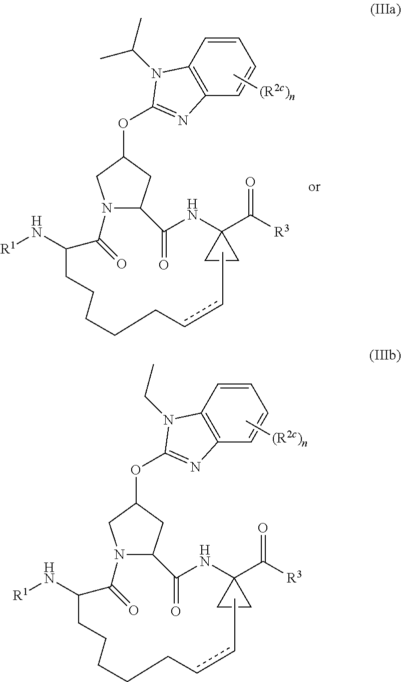

18. The compound of claim 17, wherein the compound has the structure of formula (IIIa-1):

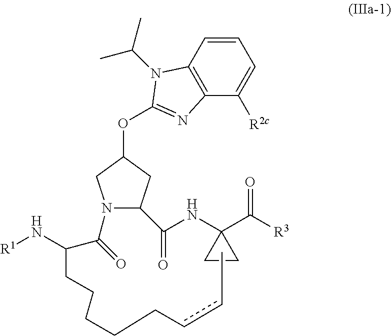

19. The compound of claim 17, wherein each R^{2c} is independently selected from the group consisting of —CF₃, —Br(bromo), —Cl(chloro), —C(O)OH, —C(O)NR′R″, —NR′R″, —NHC(O)NR′R″, —NHC(O)OR^{1c}, —NHS(O)₂R^{1c}, C₂₋₆ alkyl, C₂₋₆ alkenyl, C₁₋₆ alkoxy, polycyclic moiety, phenyl, and heteroaryl, said C₂₋₆ alkyl, C₂₋₆ alkenyl, C₁₋₆ alkoxy, polycyclic moiety, aryl, and heteroaryl each optionally substituted with one or more R¹²;
each R¹² is independently selected from the group consisting of C₁₋₆ alkyl, C₃₋₇ cycloalkyl, C₁₋₆ alkoxy, pyridinyl, phenylalkyl, phenyl, —F (fluoro), —Cl (Chloro), —CN, —CF₃, —OCF₃, —C(O)NR′R″ and morpholinyl, pyrrolidinyl, piperidiny, C₃₋₇ cycloalkyl-alkyl, wherein said C₁₋₆ alkyl, C₃₋₇ cycloalkyl, C₁₋₆ alkoxy, pyridinyl, phenylalkyl, phenyl, morpholinyl, pyrrolidinyl, piperidiny, are each optionally substituted with one or more R^{12a};
each R^{12a} is independently selected from the group consisting of —F, —Cl, C₁₋₆ alkyl, C₁₋₆ alkoxy, C₃₋₇ cycloalkyl, and aryl;
each NR′R″ is separately selected wherein R′ and R″ are each independently selected from the group consisting of —H (hydrogen), —F, —Cl, —C(O)NR′R″, C₁₋₆ alkyl, C₂₋₆ alkenyl, C₁₋₆ alkoxy, phenyl, phenylalkyl and heteroaryl; or R′ and R″ are taken together with the nitrogen to which they are attached to form heterocyclyl;

20. The compound of claim 16 selected from the group consisting of Compounds 201-204, 210-293, 1201-1222, 1401-1436, 1701-1732, and 1734-1780.

21. The compound of claim 16 having one of the following formulas:

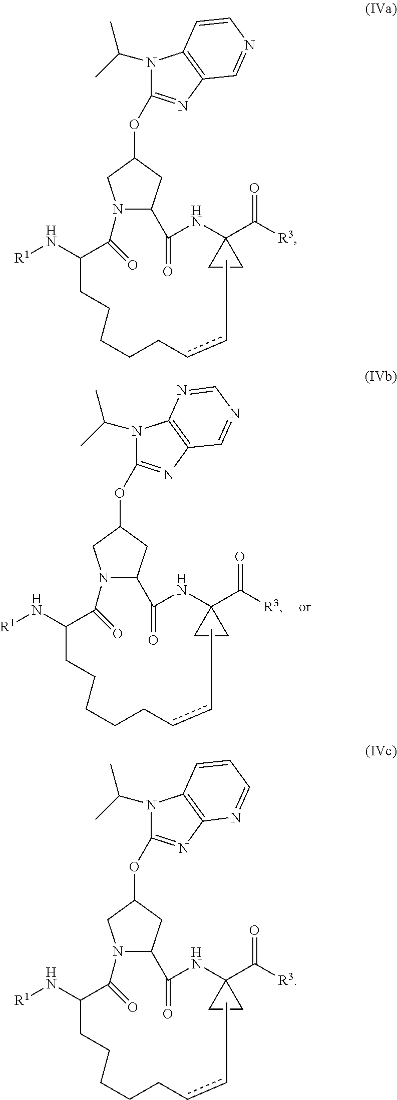

22. The compound of claim 16, wherein:
R¹ is selected from the group consisting of —C(O)O-t-butyl and phenyl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NCH(O)NR^{1a}R^{1b}, —C(O)OR^{1c}, and heteroaryl; and
R³ is —OH, —NHS(O)₂R^{3a} or —NHS(O)₂NR^{3b}R^{3c}, where R^{1a} is C₃₋₇cycloalkyl optionally substituted with methyl, and R^{3b} and R^{a}o are methyl.

23. The compound of claim 22 selected from the group consisting of Compounds 209 and 501-504.

24. A compound having the structure of Formula (V): 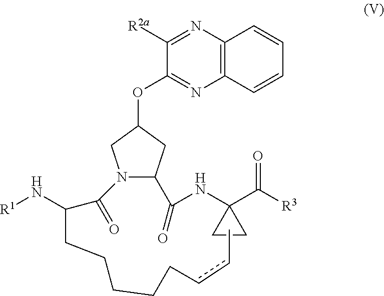
or a pharmaceutically acceptable salt or prodrug thereof wherein:
(a) R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, NHC(O)NR^{1a}R^{1b}, —C(O)OR^{1c}, and heteroaryl;
R^{1e} is selected from the group consisting of t-butyl, cycloalkyl, and heterocyclyl;
R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl;
R^{1c} and R^{1d} are each separately selected from the group consisting of —H, C₁₋₄ alkoxy, C₁₋₆ alkyl, C₃₋₇ cycloalkyl, aryl, arylalkyl and heteroaryl;
(b) R^{2a} is selected from the group consisting of —H, —C(O)OR^{1c}, C₁₋₆ alkyl optionally substituted with up to 5 fluoro, C₂₋₆ alkenyl, C₃₋₇ cycloalkyl, optionally substituted aryl and optionally substituted heteroaryl;
(c) R³ is —OH, —NHS(O)₂R^{3a}, —NHS(O)₂OR^{3a} or —NHS(O)₂NR^{3b}R^{3c}; where R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro;
wherein R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro;
or R^{3b} and R^{3c} are taken together with the nitrogen to which they are attached to form a three- to six-membered heterocyclic ring, bonded to the parent structure through a nitrogen, and the heterocylic ring is optionally substituted with one or more substituents each independently selected from the group consisting of halo, cyano, nitro, C₁₋₆ alkyl, C₁₋₆ alkoxy, and phenyl;
each t is independently 0, 1 or 2;
each q is independently 0, 1 or 2; and
(d) any bond represented by a dashed and solid line represents a bond selected from the group consisting of a single bond and a double bond.

25. The compound of claim 24 selected from the group consisting of Compounds 301-312.

26. The compound of claim 24, wherein R¹ is selected from the group consisting of —C(O)O-t-butyl, and R³ is —OH, —NHS(O)₂R^{3a} or —NHS(O)₂NR^{3b}R^{3c}, where R^{3a} is C₃₋₇cycloalkyl optionally substituted with methyl, and R^{3b} and R^{3c} are methyl.

27. A compound having the structure of one of the following formulas: 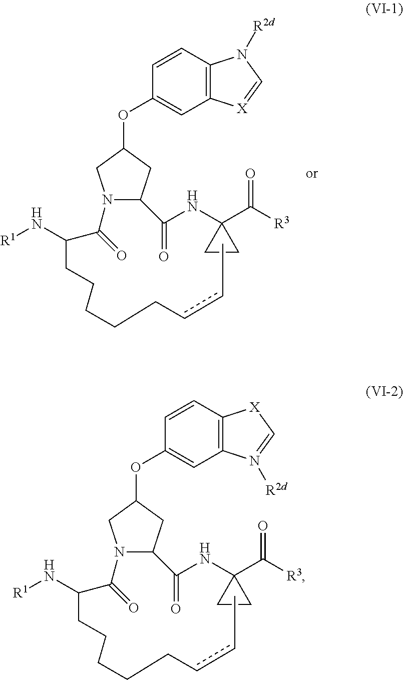
or a pharmaceutically acceptable salt or prodrug thereof wherein:
(a) R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, NHC(O)NR^{1a}R^{1b}, —C(O)OR^{1c}, and heteroaryl;
R^{1e} is selected from the group consisting of t-butyl, cycloalkyl, and heterocyclyl;
R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl;
R^{1c} and R^{1d} are each separately selected from the group consisting of —H, C₁₋₄ alkoxy, C₁₋₆ alkyl, C₃₋₇ cycloalkyl, aryl, arylalkyl and heteroaryl;
(b) X is —N— or —CH—, R^{2d} is selected from the group consisting of C₁₋₆ alkyl optionally substituted with up to 5 fluoro, C₂₋₆ alkenyl, C₃₋₇ cycloalkyl, arylalkyl, optionally substituted aryl and optionally substituted heteroaryl;
(c) R³ is —OH, —NHS(O)₂R^{3a}, —NHS(O)₂OR^{3a} or —NHS(O)₂NR^{3b}R^{3c}; where R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro;
wherein R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro;
or R^{3b} and R^{3c} are taken together with the nitrogen to which they are attached to form a three- to six-membered heterocyclic ring, bonded to the parent structure through a nitrogen, and the heterocylic ring is optionally substituted with one or more substituents each independently selected from the group consisting of halo, cyano, nitro, C₁₋₆ alkyl, C₁₋₆ alkoxy, and phenyl;
each t is independently 0, 1 or 2;
each q is independently 0, 1 or 2; and
(d) any bond represented by a dashed and solid line represents a bond selected from the group consisting of a single bond and a double bond.

28. The compound of claim 27 selected from the group consisting of Compounds 294-299 and 701-702.

29. A compound having the structure of one of the following formulas: 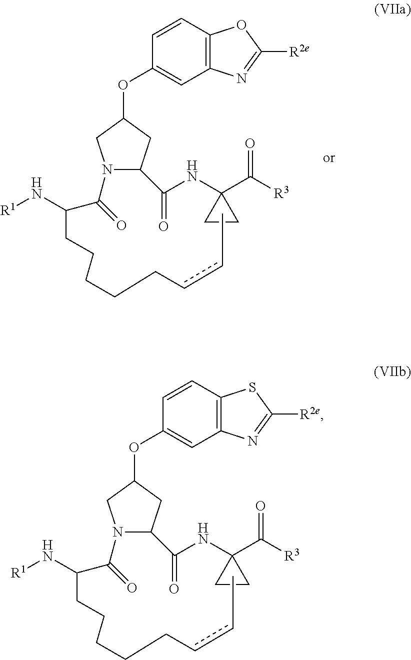
or a pharmaceutically acceptable salt or prodrug thereof wherein:
(a) R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to fluoro, C₂₋₆ alkenyl, C₂₋₆ alkynyl, —C(O)NR^{1a}R^{1b}, —NCH(O)NR^{1a}R^{1b}, C(O)OR^{1c}, and heteroaryl;
R^{1e} is selected from the group consisting of t-butyl, cycloalkyl, and heterocyclyl;
R^{1a} and R^{1b} are taken together with the nitrogen to which they are attached to form piperazinyl or morpholinyl, each optionally substituted with one or more substituents independently selected from optionally substituted C₁₋₆alkyl, C₂₋₆ alkenyl, C₂₋₆ alkynyl, —C(O)OR^{1c}, —C(O)R^{1d}, optionally substituted aryl, and optionally substituted heteroaryl;
R^{1c} and R^{1d} are each separately selected from the group consisting of —H, C₁₋₄ alkoxy, C₁₋₆ alkyl, C₃₋₇ cycloalkyl, aryl, arylalkyl and heteroaryl;
(b) R^{2e} is selected from the group consisting of —H, halo, —C(O)OR^{1c}, —C(O)NR′R″, —NR′R″, —NHC(O)NR′R″, C₁₋₆ alkyl optionally substituted with up to 5 fluoro, C₂₋₆ alkenyl, C₃₋₇ cycloalkyl, optionally substituted C₁₋₆ alkoxy, optionally substituted aryl and optionally substituted heteroaryl; wherein R′ and R″ are each independently selected from the group consisting of —H, optionally substituted C₁₋₆ alkyl, optionally substituted C₂₋₆ alkenyl, optionally substituted aryl, optionally substituted arylalkyl and optionally substituted heteroaryl;
(c) R³ is —OH, —NHS(O)₂R^{3a}, —NHS(O)₂OR^{3a} or —NHS(O)₂NR^{3b}R^{3c}; where R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro;
wherein R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro;
or R^{3b} and R^{3c} are taken together with the nitrogen to which they are attached to form a three- to six-membered heterocyclic ring, bonded to the parent structure through a nitrogen, and the heterocylic ring is optionally substituted with one or more substituents each independently selected from the group consisting of halo, cyano, nitro, C₁₋₆ alkyl, C₁₋₆ alkoxy, and phenyl;
each t is independently 0, 1 or 2;
each q is independently 0, 1 or 2; and
(d) any bond represented by a dashed and solid line represents a bond selected from the group consisting of a single bond and a double bond.

30. The compound of claim 29 selected from the group consisting of Compounds 1251-1253.

31. A compound having the structure of Formula VIIIa: 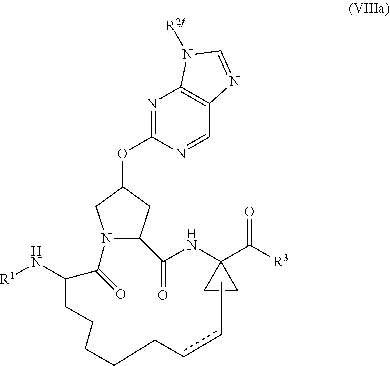
or a pharmaceutically acceptable salt or prodrug thereof wherein:
(a) R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NHC(O)NR^{1a}R^{1b}, —C(O)OR^{1c}, and heteroaryl;
R^{1e} is selected from the group consisting of t-butyl, cycloalkyl, and heterocyclyl;
R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl;
R^{1c} and R^{1d} are each separately selected from the group consisting of —H, C₁₋₄ alkoxy, C₁₋₆ alkyl, C₃₋₇ cycloalkyl, aryl, arylalkyl and heteroaryl;
(b) R^{2f} is selected from the group consisting of C₁₋₆ alkyl optionally substituted with up to 5 fluoro, C₂₋₆ alkenyl, C₃₋₇ cycloalkyl, arylalkyl, optionally substituted aryl and optionally substituted heteroaryl;
(c) R³ is —OH, —NHS(O)₂R^{3a}, —NHS(O)₂OR^{3a} or —NHS(O)₂NR^{3b}R^{3c}; where R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro;
wherein R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro;
or R^{3b} and lee are taken together with the nitrogen to which they are attached to form a three- to six-membered heterocyclic ring, bonded to the parent structure through a nitrogen, and the heterocylic ring is optionally substituted with one or more substituents each independently selected from the group consisting of halo, cyano, nitro, C₁₋₆ alkyl, C₁₋₆ alkoxy, and phenyl;
each t is independently 0, 1 or 2;
each q is independently 0, 1 or 2; and
(d) any bond represented by a dashed and solid line represents a bond selected from the group consisting of a single bond and a double bond.

32. The compound of claim 31 selected from Compound 505 or 506.

33. A compound having the structure of Formula IX: 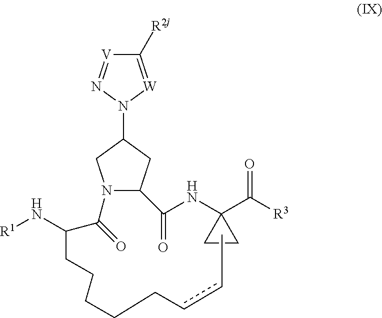
or a pharmaceutically acceptable salt or prodrug thereof wherein:
(a) R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NCH(O)NR^{1a}R^{1b}, —C(O)OR^{1c}, and heteroaryl;
R^{1e} is selected from the group consisting of t-butyl, cycloalkyl, and heterocyclyl;
R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl;
R^{1c} and R^{1d} are each separately selected from the group consisting of —H, C₁₋₄ alkoxy, C₁₋₆ alkyl, C₃₋₇ cycloalkyl, aryl, arylalkyl and heteroaryl;
(b) V and W are each independently selected from —CR^{2k}— or —N—, wherein V and W are not both —CR^{2k}—;
(c) R^{2j} and R^{2k} are each independently selected from the group consisting of H, halo, optionally substituted aryl, optionally substituted heteroaryl; or R^{2j} and R^{2k} together form an aryl ring optionally substituted by 1-3 R^{2g};
wherein R^{2g} is selected from the group consisting of —H, —Br, —Cl, —C(O)OR^{1c}, —C(O)NR′R″, —NR′R″, —NHC(O)NR′R″, C₁₋₆ alkyl optionally substituted with up to 5 fluoro, C₂₋₆ alkenyl, C₃₋₇ cycloalkyl, optionally substituted C₁₋₆ alkoxy, optionally substituted aryl and optionally substituted heteroaryl; wherein R′ and R″ are each independently selected from the group consisting of —H, optionally substituted C₁₋₆ alkyl, optionally substituted C₂₋₆ alkenyl, optionally substituted aryl, optionally substituted arylalkyl and optionally substituted heteroaryl;
(d) R³ is —OH, —NHS(O)₂R^{3a}, —NHS(O)₂OR^{3a} or —NHS(O)₂NR^{3b}R^{3c}; where R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro;
wherein R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro;
or R^{3b} and R^{3c} are taken together with the nitrogen to which they are attached to form a three- to six-membered heterocyclic ring, bonded to the parent structure through a nitrogen, and the heterocylic ring is optionally substituted with one or more substituents each independently selected from the group consisting of halo, cyano, nitro, C₁₋₆ alkyl, C₁₋₆ alkoxy, and phenyl;
each t is independently 0, 1 or 2;
each q is independently 0, 1 or 2; and
(e) any bond represented by a dashed and solid line represents a bond selected from the group consisting of a single bond and a double bond.

34. The compound of claim 33 having a formula selected from the group consisting of:

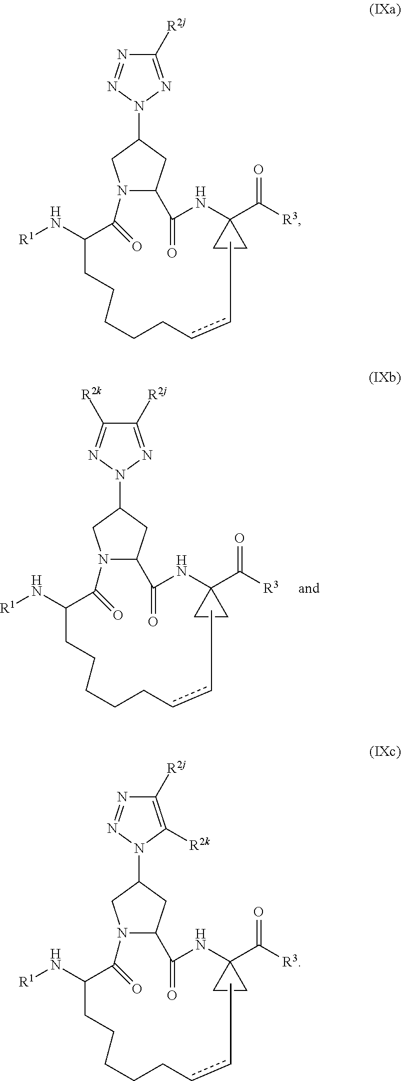

35. The compound of claim 34 selected from the group consisting of Compounds 801-805 and 1501-1506.

36. A compound having the structure of Formula (X): 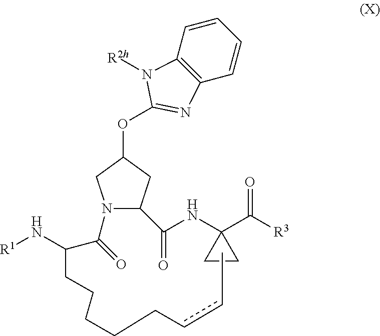
or a pharmaceutically acceptable salt or prodrug thereof wherein:
(a) R¹ is selected from the group consisting of —C(O)OR^{1e}, optionally substituted heteroaryl, and aryl optionally substituted with one or more substituents each independently selected from the group consisting of halo, amino, C₁₋₆ alkyl optionally substituted with up to 5 fluoro, C₁₋₆ alkoxy optionally substituted with up to 5 fluoro, C₂₋₆ alkenyl, C₂₋₆ alkynyl, —C(O)NR^{1a}R^{1b}, —NCH(O)NR^{1a}R^{1b}, C(O)OR^{1c}, and heteroaryl;
R^{1e} is selected from the group consisting of t-butyl, cycloalkyl, and heterocyclyl;
R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl;
R^{1c} and R^{1d} are each separately selected from the group consisting of —H, C₁₋₄ alkoxy, C₁₋₆ alkyl, C₃₋₇ cycloalkyl, aryl, arylalkyl and heteroaryl;
(b) R^{2h} is selected from the group consisting of n-propyl, cyclopropyl, n-butyl, t-butyl, 1-sec-butyl and phenyl;
(c) R³ is —OH, —NHS(O)₂R^{3a}, —NHS(O)₂OR^{3a} or —NHS(O)₂NR^{3b}R^{3c}; where R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 5 fluoro, and C₁₋₆ alkoxy optionally substituted with up to 5 fluoro;
wherein R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro;
or R^{3b} and R^{3c} are taken together with the nitrogen to which they are attached to form a three- to six-membered heterocyclic ring, bonded to the parent structure through a nitrogen, and the heterocylic ring is optionally substituted with one or more substituents each independently selected from the group consisting of halo, cyano, nitro, C₁₋₆ alkyl, C₁₋₆ alkoxy, and phenyl;
each t is independently 0, 1 or 2;
each q is independently 0, 1 or 2; and
(d) any bond represented by a dashed and solid line represents a bond selected from the group consisting of a single bond and a double bond.

37. The compound of claim 36 selected from the group consisting of Compounds 200 and 205-208.

38. A compound selected from the group consisting of:

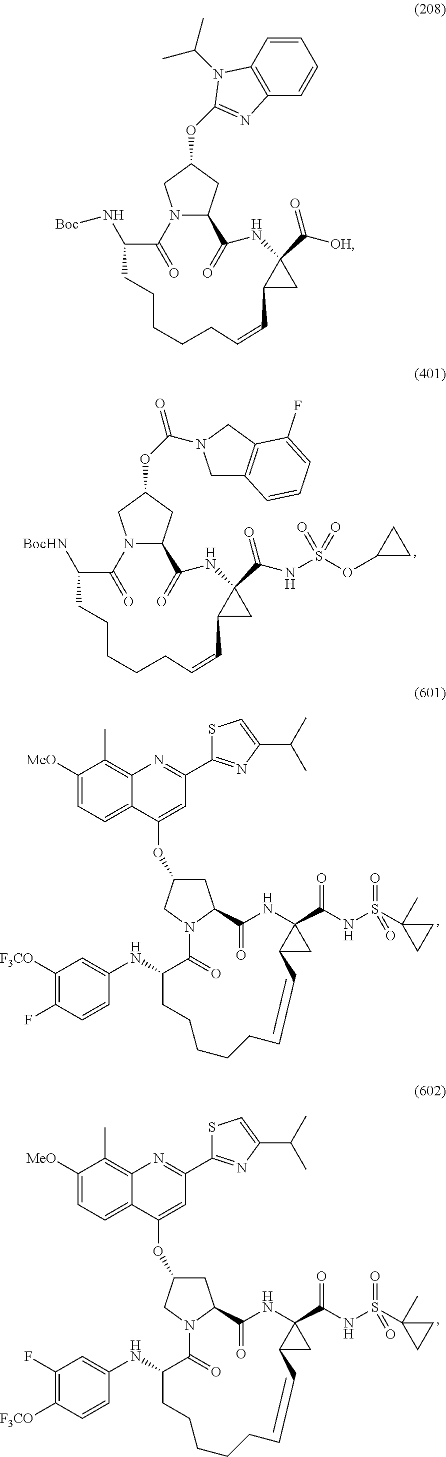

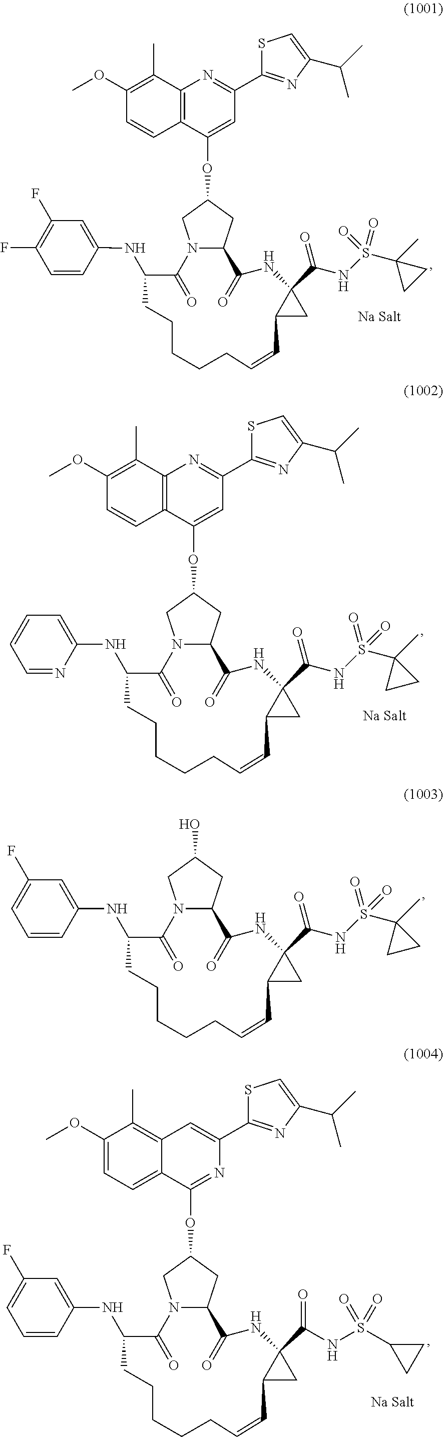

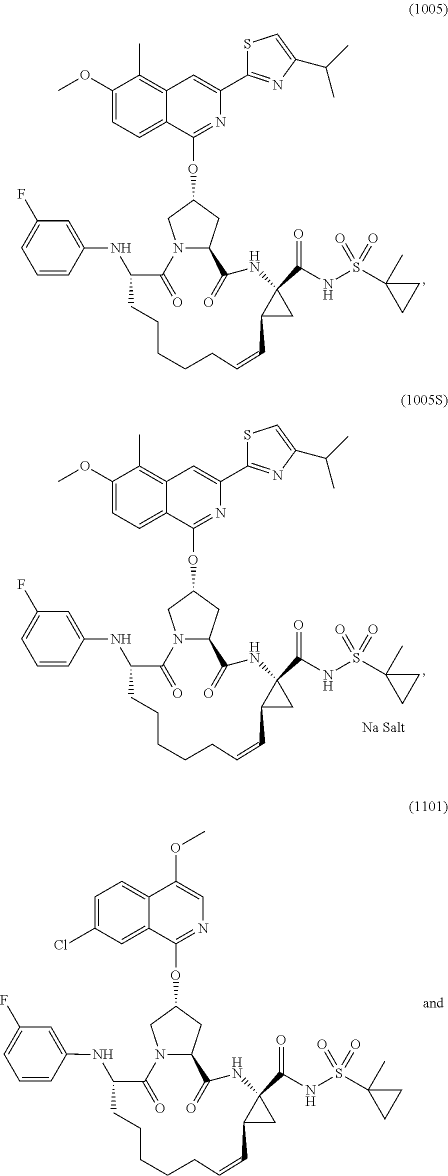

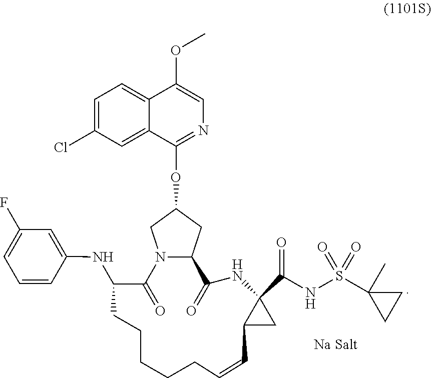

39. A pharmaceutical composition comprising a pharmaceutically acceptable excipient and a compound of claim 1.

40. A method of inhibiting NS3/NS4 protease activity comprising contacting a NS3/NS4 protease with a compound of claim 1.

41. The method of claim 40 in which the contacting is conducted in vivo.

42. The method of claim 41, further comprising identifying a subject suffering from a hepatitis C infection and administering the compound to the subject in an amount effective to treat the infection.

43. The method of claim 42, wherein the method further comprises administering to the individual an effective amount of a nucleoside analog.

44. The method of claim 43, wherein the nucleoside analog is selected from ribavirin, levovirin, viramidine, an L-nucleoside, and isatoribine.

45. The method of claim 42, wherein the method further comprises administering to the individual an effective amount of a human immunodeficiency virus 1 protease inhibitor.

46. The method of claim 45, wherein the protease inhibitor is ritonavir.

47. The method of claim 42, wherein the method further comprises administering to the individual an effective amount of an NS5B RNA-dependent RNA polymerase inhibitor.

48. The method of claim 42, wherein the method further comprises administering to the individual an effective amount of interferon-gamma (IFN-γ).

49. The method of claim 48, wherein the IFN-γ is administered subcutaneously in an amount of from about 10 μg to about 300 μg.

50. The method of claim 42, wherein the method further comprises administering to the individual an effective amount of interferon-alpha (IFN-α).

51. The method of claim 50, wherein the IFN-α is monoPEG-ylated consensus IFN-α administered at a dosing interval of every 8 days to every 14 days.

52. The method of claim 50, wherein the IFN-α is monoPEG-ylated consensus IFN-α administered at a dosing interval of once every 7 days.

53. The method of claim 50, wherein the IFN-α is INFERGEN consensus IFN-α.

54. The method of claim 42, further comprising administering an effective amount of an agent selected from 3′-azidothymidine, 2′,3′-dideoxyinosine, 2′,3′-dideoxycytidine, 2′,3′-didehydro-2′,3′-dideoxythymidine (stavudine), combivir, abacavir, adefovir dipoxil, cidofovir, and an inosine monophosphate dehydrogenase inhibitor.

55. The method of claim 42, wherein a sustained viral response is achieved.

56. The method of claim 40, in which the contacting is conducted ex vivo.

57. A method of treating liver fibrosis in an individual, the method comprising administering to the individual an effective amount of a compound of claim 1.

58. The method of claim 57, wherein the method further comprises administering to the individual an effective amount of a nucleoside analog.

59. The method of claim 58, wherein the nucleoside analog is selected from ribavirin, levovirin, viramidine, an L-nucleoside, and isatoribine.

60. The method of claim 57, wherein the method further comprises administering to the individual an effective amount of a human immunodeficiency virus 1 protease inhibitor.

61. The method of claim 60, wherein the protease inhibitor is ritonavir.

62. The method of claim 57, wherein the method further comprises administering to the individual an effective amount of an NS5B RNA-dependent RNA polymerase inhibitor.

63. The method of claim 57, wherein the method further comprises administering to the individual an effective amount of interferon-gamma (IFN-γ).

64. The method of claim 63, wherein the IFN-γ is administered subcutaneously in an amount of from about 10 μg to about 300 μg.

65. The method of claim 57, wherein the method further comprises administering to the individual an effective amount of interferon-alpha (IFN-α).

66. The method of claim 65, wherein the IFN-α is monoPEG-ylated consensus IFN-α administered at a dosing interval of every 8 days to every 14 days.

67. The method of claim 65, wherein the IFN-α is monoPEG-ylated consensus IFN-α administered at a dosing interval of once every 7 days.

68. The method of claim 65, wherein the IFN-α is INFERGEN consensus IFN-α.

69. The method of claim 57, further comprising administering an effective amount of an agent selected from 3′-azidothymidine, 2′,3′-dideoxyinosine, 2′,3′-dideoxycytidine, 2′,3′-didehydro-2′,3′-dideoxythymidine (stavudine), combivir, abacavir, adefovir dipoxil, cidofovir, and an inosine monophosphate dehydrogenase inhibitor.

70. A method of increasing liver function in an individual having a hepatitis C virus infection, the method comprising administering to the individual an effective amount of a compound of claim 1.

71. The method of claim 70, wherein the method further comprises administering to the individual an effective amount of a nucleoside analog.

72. The method of claim 71, wherein the nucleoside analog is selected from ribavirin, levovirin, viramidine, an L-nucleoside, and isatoribine.

73. The method of claim 70, wherein the method further comprises administering to the individual an effective amount of a human immunodeficiency virus 1 protease inhibitor.

74. The method of claim 73, wherein the protease inhibitor is ritonavir.

75. The method of claim 70, wherein the method further comprises administering to the individual an effective amount of an NS5B RNA-dependent RNA polymerase inhibitor.

76. The method of claim 75, wherein the method further comprises administering to the individual an effective amount of interferon-gamma (IFN-γ).

77. The method of claim 76, wherein the IFN-γ is administered subcutaneously in an amount of from about 10 μg to about 300 μg.

78. The method of claim 70, wherein the method further comprises administering to the individual an effective amount of interferon-alpha (IFN-α).

79. The method of claim 78, wherein the IFN-α is monoPEG-ylated consensus IFN-α administered at a dosing interval of every 8 days to every 14 days.

80. The method of claim 78, wherein the IFN-α is monoPEG-ylated consensus IFN-α administered at a dosing interval of once every 7 days.

81. The method of claim 78, wherein the IFN-α is INFERGEN consensus IFN-α.

82. The method of claim 70, further comprising administering an effective amount of an agent selected from 3′-azidothymidine, 2′,3′-dideoxyinosine, 2′,3′-dideoxycytidine, 2′,3′-didehydro-2′,3′-dideoxythymidine (stavudine), combivir, abacavir, adefovir dipoxil, cidofovir, and an inosine monophosphate dehydrogenase inhibitor.

83. A compound having a 50% inhibition concentration (IC₅₀) of wild-type NS3 protease of 20 nM or less and having an IC₅₀ of an NS3 protease mutated at position 155 of 200 nM or less, having the formula (XI): 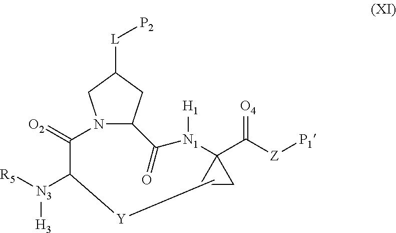
or a pharmaceutically acceptable salt, prodrug, or ester thereof wherein:
(a) Z is a group configured to hydrogen bond to an NS3 protease His57 imidazole moiety, and to hydrogen bond with the hydrogen and nitrogen of the backbone amide group of the NS3 amino acid at position 137;
(b) P₁′ is a group configured to form a non-polar interaction with at least one NS3 protease S1′ pocket moiety selected from the group consisting of Lys136, Gly137, Ser139, His57, Gly58, Gln41, Ser42, and Phe43;
(g) L is a linker group consisting of from 1 to 5 atoms selected from the group consisting of carbon, oxygen, nitrogen, hydrogen, and sulfur;
(h) P₂ is selected from the group consisting of unsubstituted aryl, substituted aryl, unsubstituted heteroaryl, substituted heteroaryl, unsubstituted heterocyclic and substituted heterocyclic; P₂ being configured to form a non-polar interaction with at least one NS3 protease S2 pocket moiety selected from the group consisting of Tyr56, Gly58, Ala59, Gly60, Gln41, His57, Va178, Asp79, Gln80 and Asp81, and P₂ being configured so that no atom of P₂ makes a nonpolar interaction with an epsilon, zeta, or eta sidechain atom of the amino acid at position 155;
(i) R⁵ is selected from the group consisting of H, C(O)NR⁶R⁷ and C(O)OR⁸;
(j)R⁶ and R⁷ are each independently H, C₁₋₆ alkyl, C₃₋₇ cycloalkyl, C₄₋₁₀ alkylcycloalkyl or phenyl, said phenyl optionally substituted by up to three halo, cyano, nitro, hydroxy, C₃₋₇ cycloalkyl, C₄₋₁₀ alkylcycloalkyl, C₂₋₆ alkenyl, hydroxy-C₁₋₆ alkyl, C₁₋₆ alkyl optionally substituted with up to 5 fluoro, C₁₋₆ alkoxy optionally substituted with up to 5 fluoro; or R⁶ and R⁷ are taken together with the nitrogen to which they are attached to form indolinyl, pyrrolidinyl, piperidinyl, piperazinyl, or morpholinyl;
(k) R⁸ is C₁₋₆ alkyl, C₃₋₇ cycloalkyl, C₄₋₁₀ alkylcycloalkyl, which are all optionally substituted from one to three times with halo, cyano, nitro, hydroxy, C₁₋₆ alkoxy, or phenyl; or R⁸ is C_{6 or 10} aryl which is optionally substituted by up to three halo, cyano, nitro, hydroxy, C₃₋₇ cycloalkyl, C₄₋₁₀ alkylcycloalkyl, C₂₋₆ alkenyl, C₁₋₆ alkoxy, hydroxy-C₁₋₆ alkyl, C₁₋₆ alkyl optionally substituted with up to 5 fluoro, C₁₋₆ alkoxy optionally substituted with up to 5 fluoro; or R⁸ is C₁₋₆ alkyl optionally substituted with up to 5 fluoro groups; or R⁸ is a tetrahydrofuran ring linked through the C₃ or C₄ position of the tetrahydrofuran ring; or R⁸ is a tetrapyranyl ring linked through the C₄ position of the tetrapyranyl ring;
(l) Y is a C₅₋₇ saturated or unsaturated chain optionally containing one or two heteroatoms selected from O, S, or NR⁹R¹⁰; and
(m) R⁹ and R¹⁰ are each independently H, C₁₋₆ alkyl, C₃₋₇ cycloalkyl, C₄₋₁₀ cycloalkyl-alkyl, or substituted or unsubstituted phenyl; or R⁹ and R¹⁰ are taken together with the nitrogen to which they are attached to form indolinyl, pyrrolidinyl, piperidinyl, piperazinyl, or morpholinyl.
